(19) United States
(12) Patent Application Publication
(10) Pub. No.: US 2025/0152719 A1
(43) Pub. Date: May 15, 2025

(54) FUSED IMIDE DERIVATIVE

(71) Applicant: Chia Tai Tianqing Pharmaceutical Group Co., Ltd., Lianyungang (CN)

(72) Inventors: Yinsheng Zhang, Lianyungang (CN); Jing Ren, Lianyungang (CN); Jinan Wang, Lianyungang (CN); Sheng Xu, Lianyungang (CN); Xiaojun Yang, Lianyungang (CN); Li Deng, Lianyungang (CN); Yongkang Huang, Lianyungang (CN); Qinglin Wang, Lianyungang (CN)

(21) Appl. No.: 18/708,972

(22) PCT Filed: Nov. 18, 2022
(86) PCT No.: PCT/CN2022/132769

§ 371 Date: May 9, 2024

(30) Foreign Application Priority Data
Nov. 18, 2021 (CN) 202111371514.4
Jul. 13, 2022 (CN) 202210824373.5
Nov. 4, 2022 (CN) 202211378992.2
Nov. 11, 2022 (CN) 202211413934.9

(51) Int. Cl. A61K 47/54

(52) U.S. Cl. CPC A61K 47/545

(57) ABSTRACT

Provided are a fused imide derivative as shown in formula I, a preparation method therefor, a pharmaceutical composition containing same, and the use thereof in the treatment of relevant diseases (such as cancers).

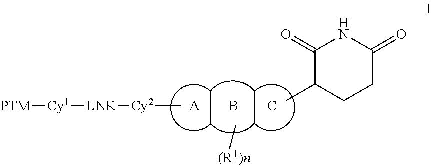

### CROSS-REFERENCE TO RELATED APPLICATIONS

The present application is an international application of Chinese Patent Application Nos. 202111371514.4, 202111667477.1, 202210824373.5, 202211261799.0, 202211378992.2, and 202211413934.9 filed on Nov. 18, 2021, Dec. 31, 2021, Jul. 13, 2022, Oct. 14, 2022, Nov. 4, 2022, and Nov. 11, 2022, respectively, and claims the priority and benefits of the Chinese Patent Applications. The disclosures of the Chinese Patent Applications are incorporated herein by reference in their entirety.

### TECHNICAL FIELD

The present application relates to a fused imide derivative and protein degrader, a preparation method therefor, a pharmaceutical composition comprising the same, and use thereof in treating a related disease (such as cancer).

### BACKGROUND

Bruton's tyrosine kinase (BTK) is mainly expressed in B cells, distributed in the lymphatic, hematopoietic and blood systems, and is a member of the non-receptor type tyrosine kinase Tec family, which also includes TEC, ITK/TSK/EMT, TXK and BMX, which have high structural homology. In recent years, researches on B cells, particularly on B cell non-Hodgkin lymphoma and rheumatoid arthritis find that BTK is often abnormally expressed. Since BTK is mainly expressed in B cells and marrow cells, BTK is a target with relatively good targeting property and safety.

Proteolysis targeting chimera (Protac) molecule is a bifunctional compound capable of binding to a target protein and E3 ubiquitin ligase simultaneously. Such compounds can induce the target protein to be recognized by proteasomes of cells, cause the degradation of the target protein and effectively reduce the content of the target protein in the cells. The introduction of ligands capable of binding different target proteins into Protac molecules has made it possible to apply Protac technology to the treatment of various diseases, and this technology has also received much attention in recent years.

### SUMMARY

In one aspect, the present application relates to a compound of formula I, a stereoisomer thereof or a pharmaceutically acceptable salt thereof:

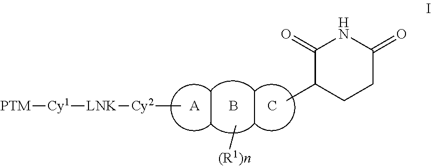

wherein,
ring A is absent or selected from the group consisting of C₅₋₁₀ cycloalkenyl, 5- to 10-membered heterocycloalkenyl, phenyl, and 5- to 6-membered heteroaryl, or
ring A is absent or selected from the group consisting of C₅₋₆ cycloalkenyl, 5- to 10-membered heterocycloalkenyl, phenyl, and 5- to 6-membered heteroaryl;
ring B is selected from phenyl;
ring C is selected from the group consisting of isoxazolyl and furanyl;
each R¹ is independently selected from the group consisting of halogen, —OH, —NH₂, —CN, C₁₋₄ alkyl, C₁₋₄ alkoxy, and C₁₋₄ haloalkyl;
n is selected from the group consisting of 0, 1, 2, and 3;
Cy¹ is selected from the group consisting of a bond, C₃₋₁₂ cycloalkyl, and 4- to 12-membered heterocycloalkyl, wherein the C₃₋₁₂ cycloalkyl or 4- to 12-membered heterocycloalkyl is optionally substituted with one or more R^{a};
LNK is selected from the group consisting of a bond, C₁₋₁₂ alkylene, and C₁₋₁₂ heteroalkylene;
Cy² is absent or selected from the group consisting of C₃₋₁₂ cycloalkyl and 4- to 12-membered heterocycloalkyl, wherein the cycloalkyl or heterocycloalkyl is optionally substituted with one or more R^{b};
each R^{a} and each R^{b} are independently selected from the group consisting of halogen, —OH, —NH₂, —CN, C₁₋₄ alkyl, C₁₋₄ alkoxy, C₁₋₄ haloalkyl, C₁₋₄ alkylamino, and di-C₁₋₄ alkylamino;
PTM is selected from the group consisting of drugs and derivatives thereof that bind to a target protein.

In some embodiments, the present application relates to a compound of formula I, a stereoisomer thereof, or a pharmaceutically acceptable salt thereof,

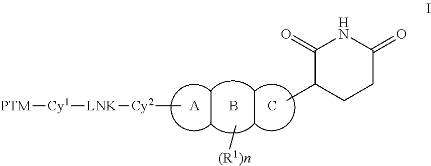

wherein,
ring A is absent or selected from the group consisting of C₅₋₆ cycloalkenyl, 5- to 10-membered heterocycloalkenyl, phenyl, and 5- to 6-membered heteroaryl;
ring B is selected from phenyl;
ring C is selected from the group consisting of isoxazolyl and furanyl;
each R¹ is independently selected from the group consisting of halogen, —OH, —NH₂, —CN, C₁₋₄ alkyl, C₁₋₄ alkoxy, and C₁₋₄ haloalkyl;
n is selected from the group consisting of 0, 1, 2, and 3;
Cy¹ is selected from the group consisting of a bond, C₃₋₁₂ cycloalkyl, and 4- to 12-membered heterocycloalkyl, wherein the C₃₋₁₂ cycloalkyl or 4- to 12-membered heterocycloalkyl is optionally substituted with one or more R^{a};
LNK is selected from the group consisting of a bond, C₁₋₁₂ alkylene, and C₁₋₁₂ heteroalkylene;
Cy² is absent or selected from the group consisting of C₃₋₁₂ cycloalkyl and 4- to 12-membered heterocycloalkyl, wherein the cycloalkyl or heterocycloalkyl is optionally substituted with one or more R^{b};
each R^{a} and each R^{b} are independently selected from the group consisting of halogen, —OH, —NH₂, —CN, C₁₋₄ alkyl, C₁₋₄ alkoxy, C₁₋₄ haloalkyl, C₁₋₄ alkylamino, and di-C₁₋₄ alkylamino;
PTM is selected from the group consisting of drugs and derivatives thereof that bind to a target protein.

In some embodiments, in the compound of formula I, the stereoisomer thereof, or the pharmaceutically acceptable salt thereof, the structural moiety

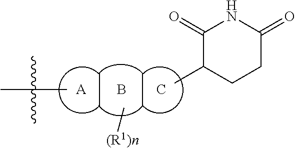

is selected from the group consisting of

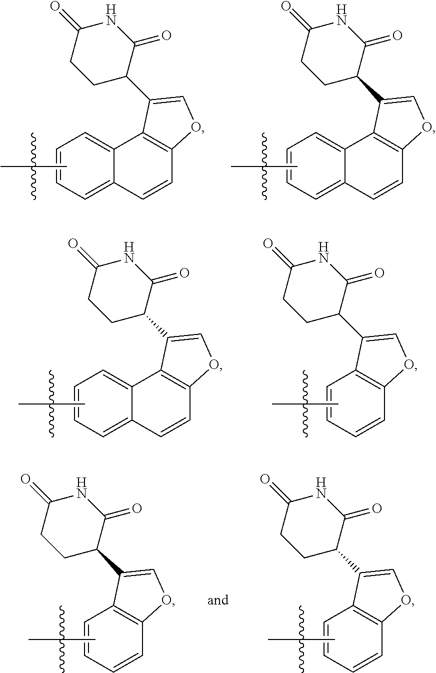

and PTM is not selected from the group consisting of the following structural moieties:

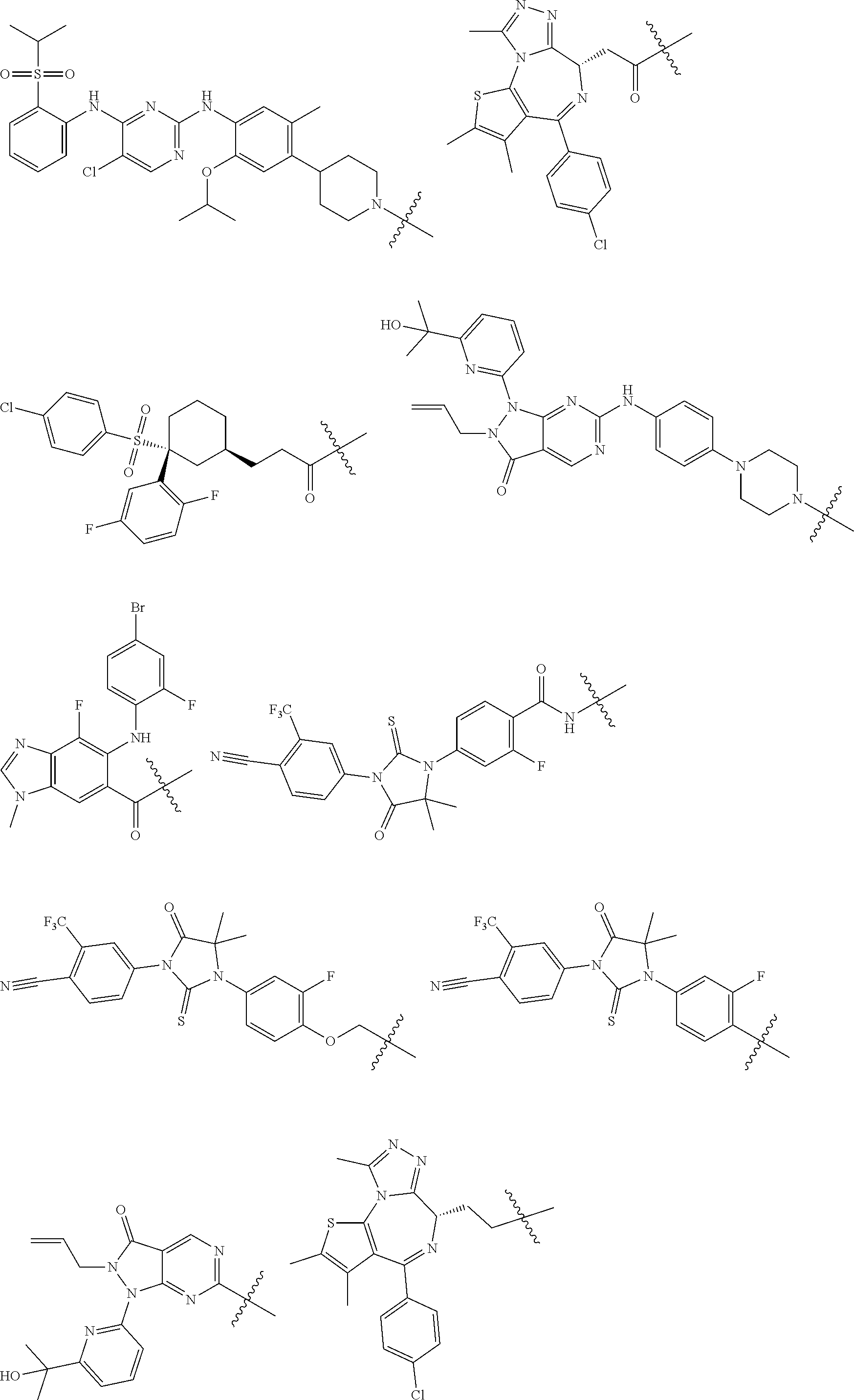

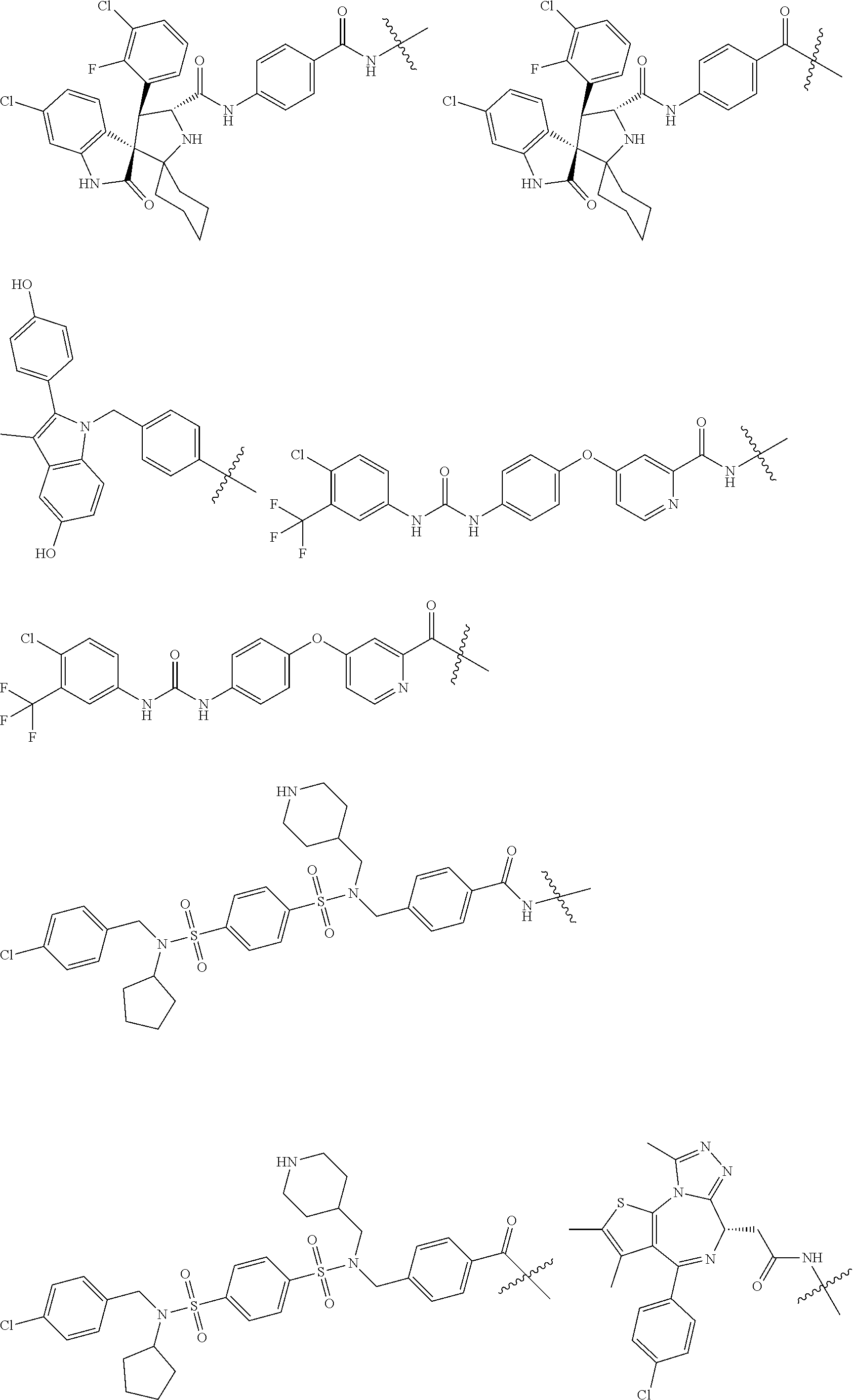

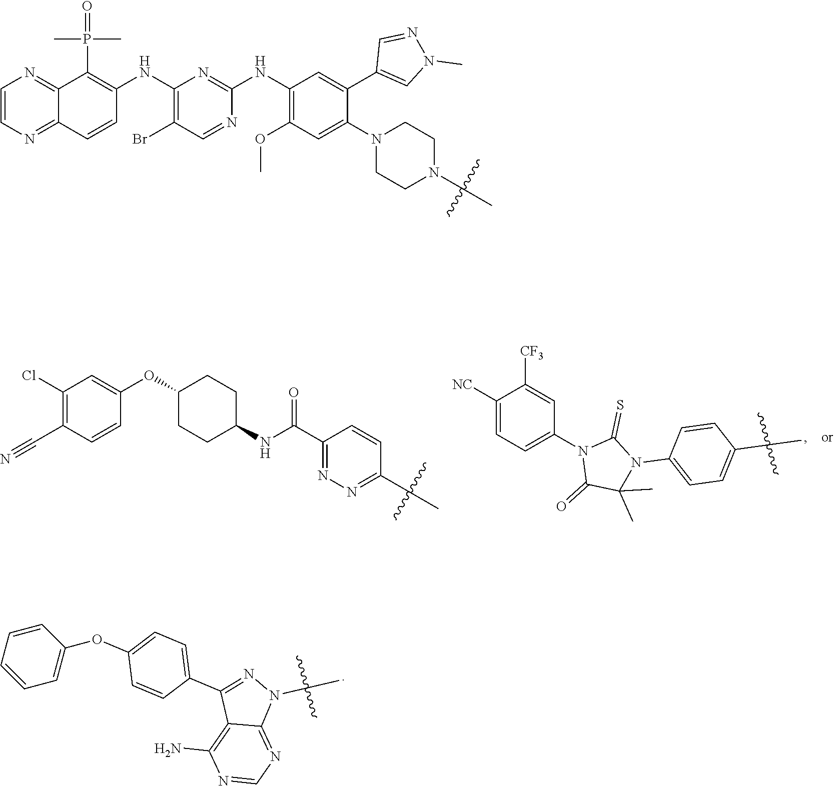 [SMILES: *N1CCN(c2cc(OC)c(Nc3ncc(Br)c(Nc4ccc5nccnc5c4P(C)(C)=O)n3)cc2-c2cnn(C)c2)CC1 ; *c1ccc(C(=O)N[C@H]2CC[C@H](Oc3ccc(C#N)c(Cl)c3)CC2)nn1 ; *c1ccc(N2C(=S)N(c3ccc(C#N)c(C(F)(F)F)c3)C(=O)C2(C)C)cc1 ; *n1nc(-c2ccc(Oc3ccccc3)cc2)c2c(N)ncnc21]

In some other embodiments, Cy¹ is selected from the group consisting of 4- to 12-membered heterocycloalkyl optionally substituted with one or more R^{a}.

In some embodiments of the present application, when A is present, Cy² or LNK (when Cy² is absent) may be directly covalently linked to ring B; likewise

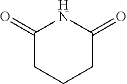

may also be directly covalently linked to ring B.

In some embodiments, PTM described herein is selected from the group consisting of drugs and derivatives thereof that act on AR, ER, kinase, phosphatase, MDM2, human BET bromodomain protein, Hsp90, HDAC, human lysine methyltransferase, RAF receptor, FKBP, angiogenic factor, immunosuppression-related receptor or protein, arene receptor, thyroid hormone receptor, HIV protease, HIV integrase, HCV protease, HBV protease, or acyl protein thioesterase 1 and/or acyl protein thioesterase 2.

In some embodiments, PTM described herein is selected from the group consisting of drugs and derivatives thereof that act on ALK, BET, CDK, PARP, EGFR, γ-secretase, CBFβ-SMMHC, WEE1, MEK, BCR-ABL, MET, RAS, BTK, VEGFR, JAK, HER2, HDAC, Akt, PI3K, mTOR, AR, ER, PDEB, SRC, MDM2, RAF, IRAK4, STAT3, and c-Myc.

In some embodiments, PTM described herein is selected from the group consisting of drugs and derivatives thereof that act on ALK, BRD4, CDK4/6, PARP, EGFR, γ-secretase, CBFβ-SMMHC, WEE1, MEK, BCR-ABL, MET, KRAS, EGFR, BTK, AR, ER, PDEB, JAK, MDM2, or RAF.

In some embodiments, PTM described herein is selected from the group consisting of drugs and derivatives thereof that act on BTK or WEE1.

In some embodiments, PTM described herein is selected from the group consisting of drugs and derivatives thereof that act on BTK.

In another aspect, the present application relates to a compound of formula II-1, a stereoisomer thereof, or a pharmaceutically acceptable salt thereof,

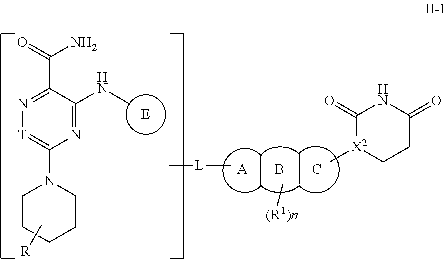

wherein,
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl;
ring E is selected from the group consisting of phenyl, benzocycloalkenyl (e.g., benzo 4- to 12-membered cycloalkenyl, or benzo 4- to 10-membered cycloalkenyl, or benzo 4- to 8-membered cycloalkenyl), and benzoheterocycloalkenyl (e.g., benzo 4- to 12-membered heterocycloalkenyl or benzo 5- to 11-membered heterocycloalkenyl);
X² is selected from the group consisting of CH and N;
L is selected from a connecting group;
ring A, ring B, ring C, R¹, and n are as defined herein.

In another aspect, the present application relates to a compound of formula II, a stereoisomer thereof, or a pharmaceutically acceptable salt thereof,

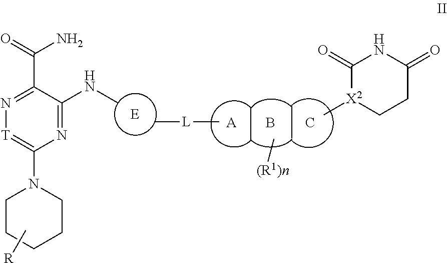

wherein,
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl;
ring E is selected from the group consisting of phenyl, benzocycloalkenyl, and benzoheterocycloalkenyl;
X² is selected from the group consisting of CH and N;
L is selected from a connecting group;
ring A, ring B, ring C, R¹, and n are as defined herein.

In some embodiments, L is selected from the group consisting of -Cy¹-LNK-Cy²-LNK-, -Cy¹-LNK-Cy²-, and -Cy¹-Cy²-LNK-, wherein Cy¹, LNK, and Cy² are as described herein. In some embodiments, L is selected from -Cy¹-LNK-Cy²-, wherein Cy¹, LNK, and Cy² are as described herein.

In another aspect, the present application relates to a compound of formula I′a, a stereoisomer thereof, or a pharmaceutically acceptable salt thereof,

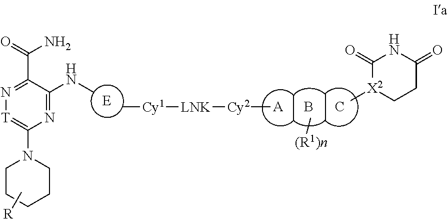

wherein,
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl;
ring E is selected from the group consisting of phenyl, benzocycloalkenyl, and benzoheterocycloalkenyl;
X² is selected from the group consisting of CH and N;
ring A, ring B, ring C, R¹, n, Cy¹, LNK, and Cy² are as defined herein.

In some embodiments, X² is selected from CH; in some embodiments, X² is selected from N.

In some embodiments, ring E is selected from the group consisting of phenyl, benzo C₅₋₁₂ cycloalkenyl, and benzo 5- to 12-membered heterocycloalkenyl.

In some embodiments, ring E is selected from the group consisting of phenyl, benzo C₅₋₆ cycloalkenyl, and benzo 5- to 11-membered heterocycloalkenyl.

In some embodiments, ring E is selected from the group consisting of phenyl, benzo 5-membered heterocycloalkenyl, benzo 6-membered heterocycloalkenyl, benzo 10-membered heterocycloalkenyl, and benzo 11-membered heterocycloalkenyl.

In some embodiments, ring E is selected from the group consisting of phenyl,

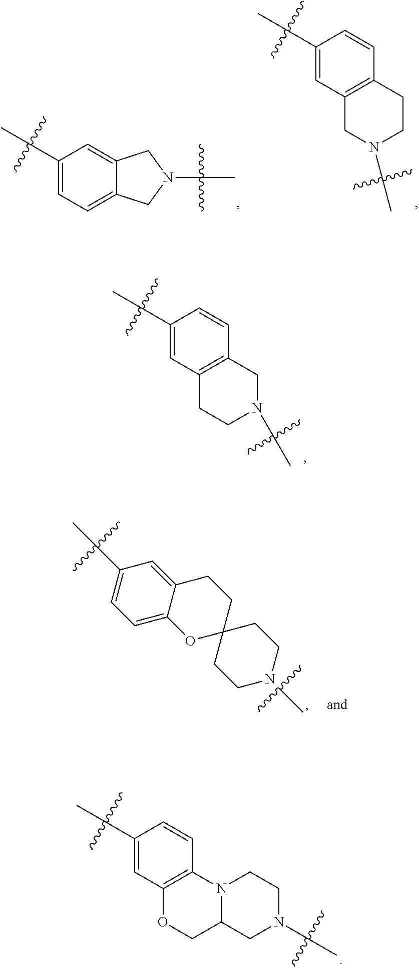

In some specific embodiments, ring E is selected from phenyl. In some specific embodiments, ring E is selected from the group consisting of

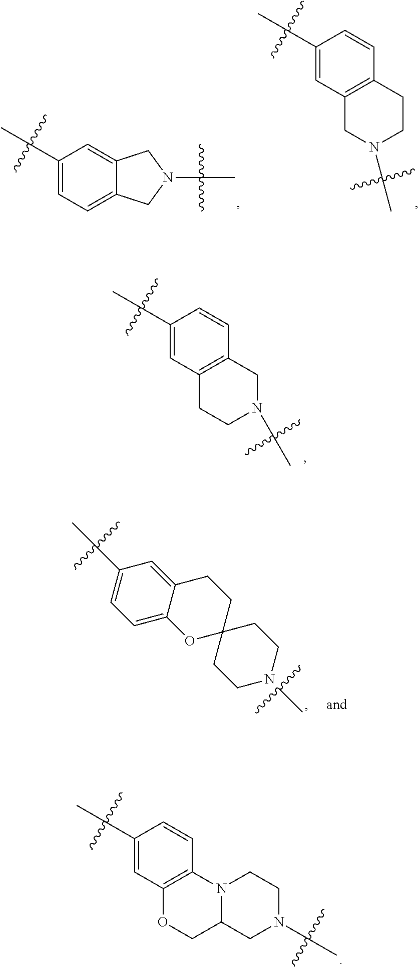

In another aspect, the compound of formula I, the stereoisomer thereof, or the pharmaceutically acceptable salt thereof described herein is selected from the group consisting of a compound of formula I′, a stereoisomer thereof, and a pharmaceutically acceptable salt thereof,

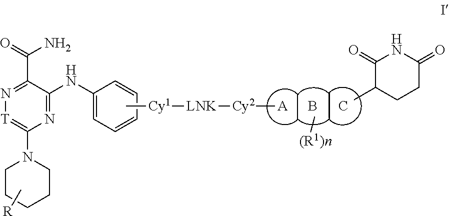

wherein,
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl.

In some embodiments, ring A is absent or selected from the group consisting of C₅₋₁₀ cycloalkenyl, 5- to 10-membered heterocycloalkenyl, phenyl, and 5- to 6-membered heteroaryl.

In some embodiments, ring A is absent or selected from the group consisting of C₅₋₈ cycloalkenyl, 5- to 10-membered heterocycloalkenyl, phenyl, and 5- to 6-membered heteroaryl.

In some embodiments, ring A is absent or selected from the group consisting of C₅₋₇ cycloalkenyl, 5- to 10-membered heterocycloalkenyl, phenyl, and 5- to 6-membered heteroaryl.

In some embodiments, ring A is absent or selected from the group consisting of C₅₋₆ cycloalkenyl, 5- to 10-membered heterocycloalkenyl, phenyl, and 5- to 6-membered heteroaryl.

In some embodiments, ring A is absent or selected from the group consisting of C₅₋₆ cycloalkenyl, 5- to 9-membered heterocycloalkenyl, phenyl, and 5-membered heteroaryl.

In some embodiments, ring A is absent or selected from the group consisting of C₅₋₆ cycloalkenyl, 5- to 9-membered heterocycloalkenyl, phenyl, pyrrolyl, pyrazolyl, furanyl, and oxazolyl.

In some embodiments, ring A is absent or selected from the group consisting of C₅ cycloalkenyl, C₆ cycloalkenyl, 5-, 6-, 7-, 8-, or 9-membered heterocycloalkenyl, phenyl, pyrrolyl, pyrazolyl, furanyl, and oxazolyl.

In some embodiments, ring A is absent or selected from the group consisting of cyclopentenyl, bicyclohexenyl, dihydropyrrolyl, tetrahydropyridinyl, tetrahydroazepinyl, dihydrooxazinyl, azaspirooctenyl, azaspirononenyl, phenyl, pyrrolyl, pyrazolyl, furanyl, and oxazolyl.

In some specific embodiments, ring A is selected from the group consisting of C₅₋₁₀ cycloalkenyl and 5- to 10-membered heterocycloalkenyl. In some specific embodiments, ring A is selected from the group consisting of C₅₋₈ cycloalkenyl and 5- to 9-membered heterocycloalkenyl. In some specific embodiments, ring A is selected from the group consisting of C₅₋₇ cycloalkenyl and 5- to 9-membered heterocycloalkenyl. In some specific embodiments, ring A is selected from the group consisting of C₅₋₆ cycloalkenyl and 5- to 9-membered heterocycloalkenyl. In some specific embodiments, ring A is selected from 5- to 9-membered heterocycloalkenyl. In some specific embodiments, ring A is selected from 5- to 8-membered heterocycloalkenyl. In some specific embodiments, ring A is selected from 5-membered heterocycloalkenyl. In some specific embodiments, ring A is selected from 6-membered heterocycloalkenyl. In some specific embodiments, ring A is selected from 7-membered heterocycloalkenyl. In some specific embodiments, ring A is selected from 8-membered heterocycloalkenyl. In some specific embodiments, ring A is selected from 9-membered heterocycloalkenyl. In some specific embodiments, ring A is absent or selected from the group consisting of C₅₋₆ cycloalkenyl, 5- to 9-membered heterocycloalkenyl (preferably 5- to 8-membered heterocycloalkenyl or 5- to 7-membered heterocycloalkenyl), and phenyl. In some preferred embodiments, ring A is absent or selected from the group consisting of C₅₋₆ cycloalkenyl, 5- to 7-membered heterocycloalkenyl containing 1-2 heteroatoms selected from the group consisting of N, O, and S (preferably containing 1-2 N atoms, e.g., 1 N atom), and phenyl. In some specific embodiments, ring A is absent or selected from the group consisting of cyclopentenyl, dihydropyrrolyl, tetrahydropyridinyl, tetrahydroazepinyl, and phenyl.

In some specific embodiments, ring A is selected from the group consisting of cyclopentenyl, bicyclohexenyl, dihydropyrrolyl, tetrahydropyridinyl, tetrahydroazepinyl, dihydrooxazinyl, azaspirooctenyl, and azaspirononenyl.

In some specific embodiments, ring A is selected from the group consisting of cyclopentenyl and bicyclohexenyl.

In some specific embodiments, ring A is selected from the group consisting of dihydropyrrolyl, dihydrooxazinyl, tetrahydropyridinyl, tetrahydroazepinyl, azaspirooctenyl, and azaspirononenyl.

In some specific embodiments, ring A is selected from the group consisting of phenyl, pyrrolyl, pyrazolyl, furanyl, and oxazolyl.

In some embodiments, ring A is absent or selected from the group consisting of C₅₋₇ cycloalkenyl, 5- to 8-membered heterocycloalkenyl, phenyl, and pyrrolyl.

In some other embodiments, ring A is absent or selected from the group consisting of C₅₋₆ cycloalkenyl, 5- to 8-membered heterocycloalkenyl, phenyl, and pyrrolyl.

In some other embodiments, ring A is absent or selected from the group consisting of C₅ cycloalkenyl, 5-, 6-, 7-, or 8-membered heterocycloalkenyl, phenyl, and pyrrolyl.

In some other embodiments, ring A is absent or selected from the group consisting of cyclopentenyl, dihydropyrrolyl, tetrahydropyridinyl, tetrahydroazepinyl, azaspirooctene, phenyl, and pyrrolyl.

In some other embodiments, ring A is absent.

In some other embodiments, ring A is selected from the group consisting of C₅₋₆ cycloalkenyl and 5- to 8-membered heterocycloalkenyl.

In some other embodiments, ring A is selected from the group consisting of cyclopentenyl, dihydropyrrolyl, tetrahydropyridinyl, tetrahydroazepinyl, and azaspirooctene. In some other embodiments, ring A is selected from cyclopentenyl. In some other embodiments, ring A is selected from the group consisting of dihydropyrrolyl, tetrahydropyridinyl, tetrahydroazepinyl, and azaspirooctene.

In some other embodiments, ring A is selected from the group consisting of phenyl and pyrrolyl.

In some embodiments, the structural moiety

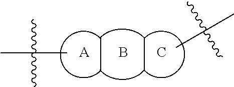

is selected from the group consisting of

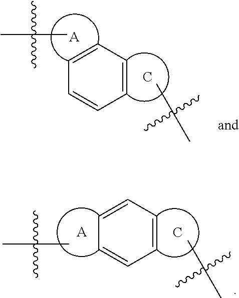

Alternatively, in some embodiments, the structural moiety

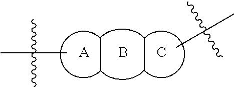

is selected from the group consisting of

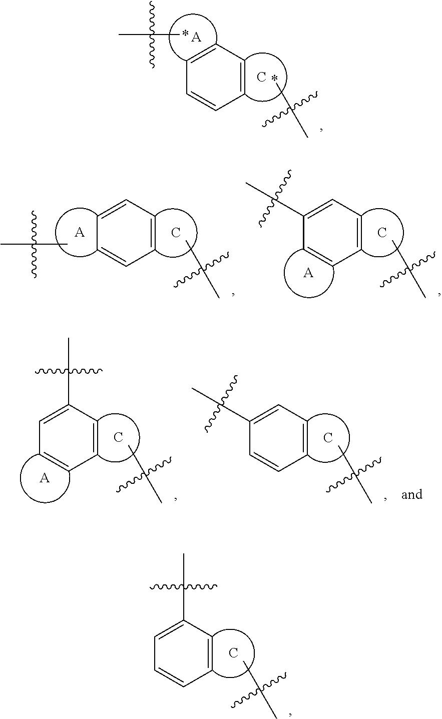

wherein * in the bond

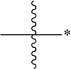 [SMILES: **]

linked to ring A indicates that the bond is linked to an atom on ring A; * in the bond

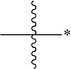 [SMILES: **]

linked to ring C indicates that the bond is linked to an atom on ring C (hereinafter, a similarly positioned * indicates the same or similar meaning).

In some embodiments, the structural moiety

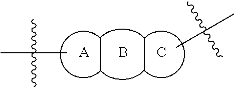

is selected from the group consisting of

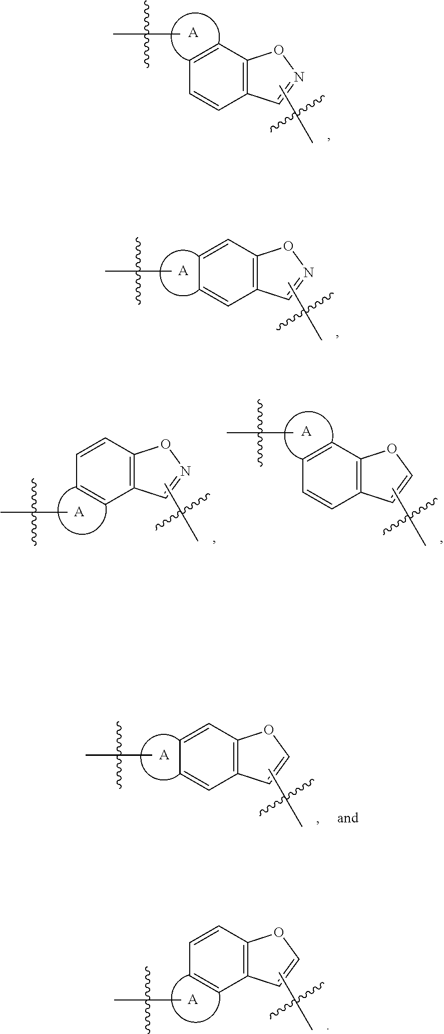

In some embodiments, the structural moiety

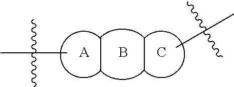

is selected from the group consisting of

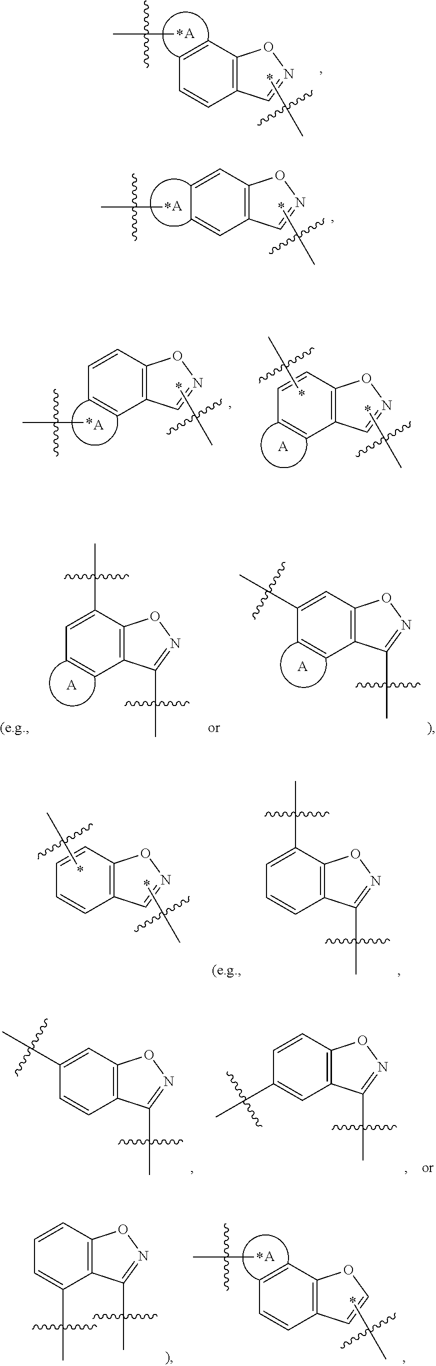

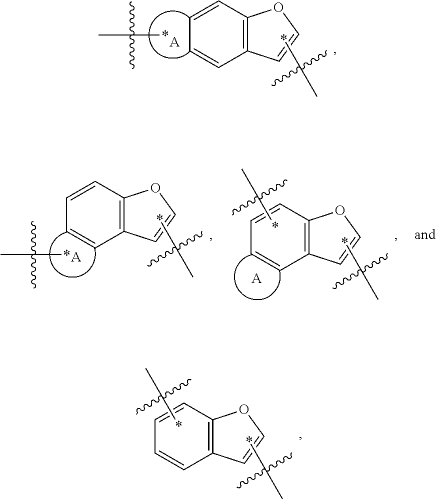

wherein * is defined in the same or similar way as for * described above.

In some specific embodiments, the structural moiety

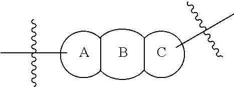

is selected from the group consisting of

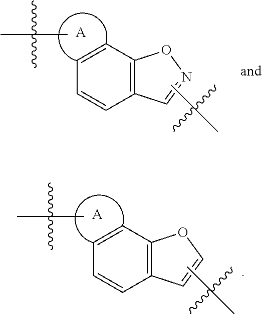

In some specific embodiments, the structural moiety

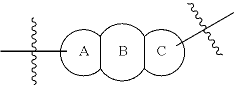

is selected from the group consisting of

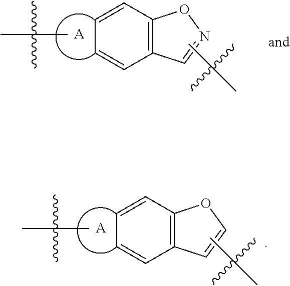

In some specific embodiments, the structural moiety

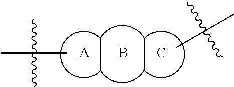

is selected from the group of

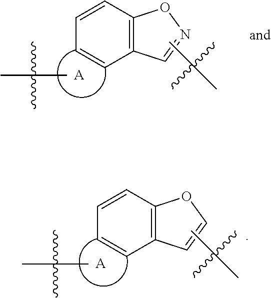

In some embodiments, the structural moiety

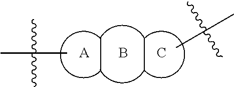

is selected from the group consisting of

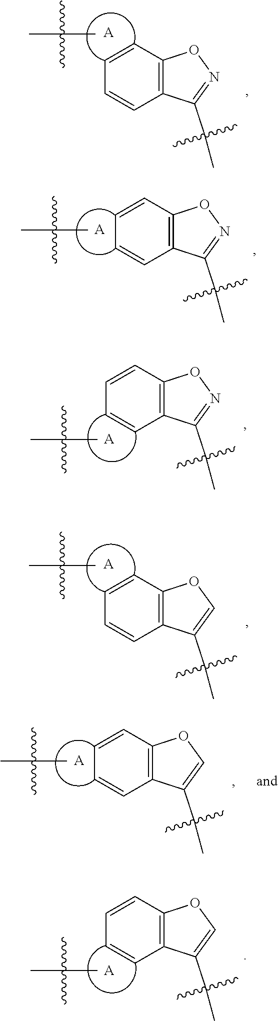

In some specific embodiments, the structural moiety

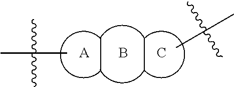

is selected from the group consisting of

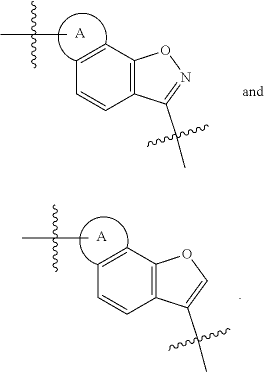

In some specific embodiments, the structural moiety

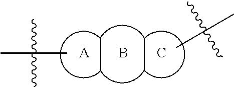

is selected from the group consisting of

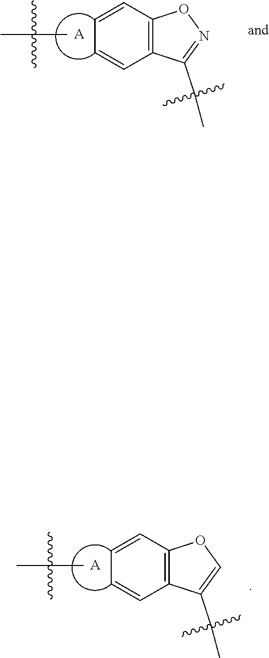

In some specific embodiments, the structural moiety

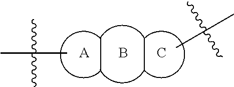

is selected from the group consisting of

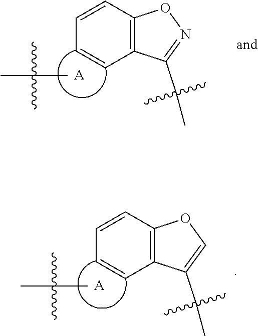

In some embodiments, the structural moiety

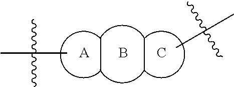

is selected from the group consisting of

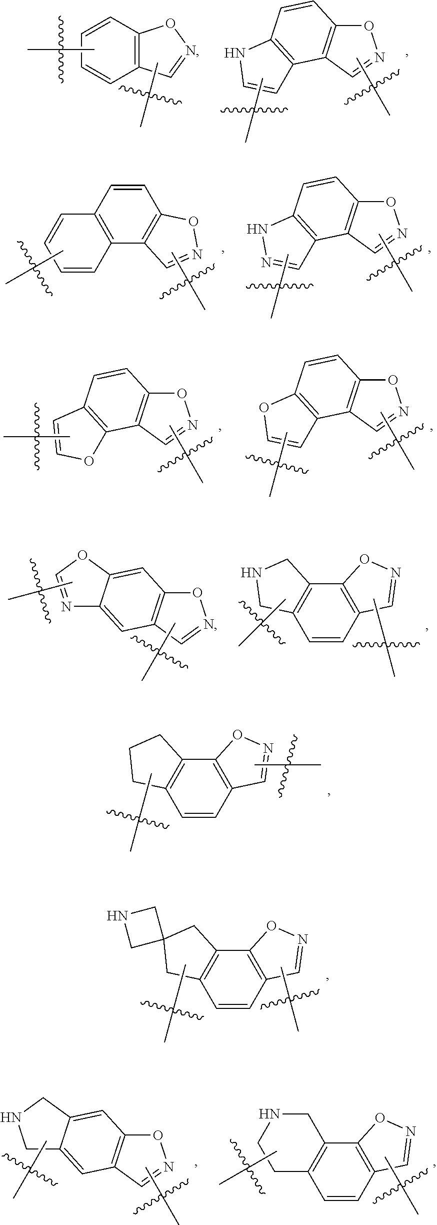

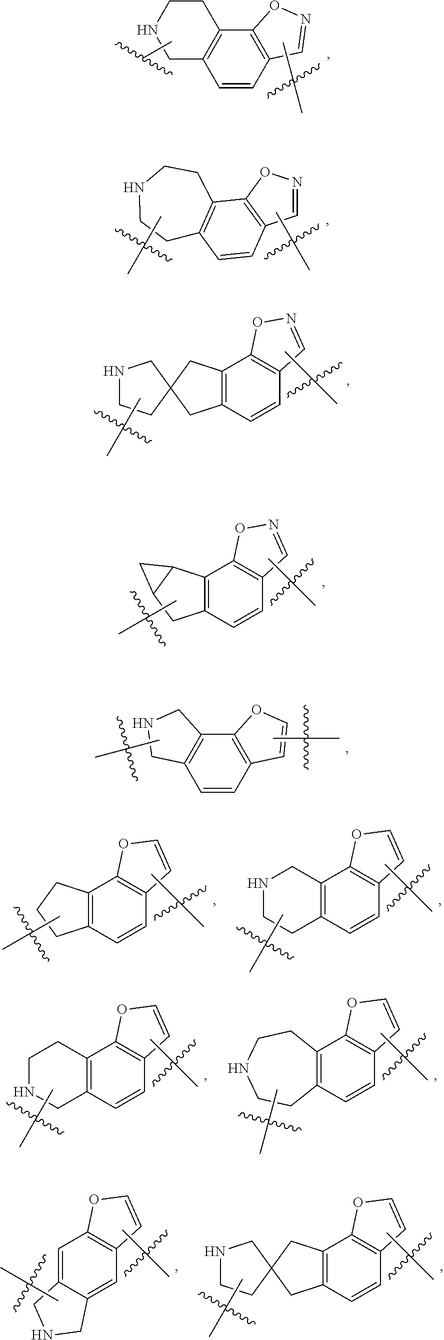

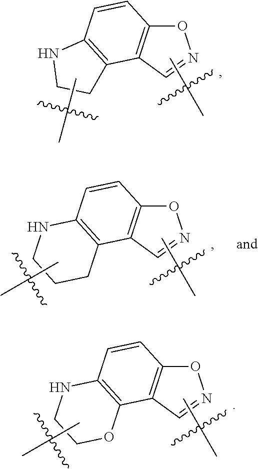

In some specific embodiments, the structural moiety

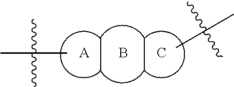

is selected from the group consisting of

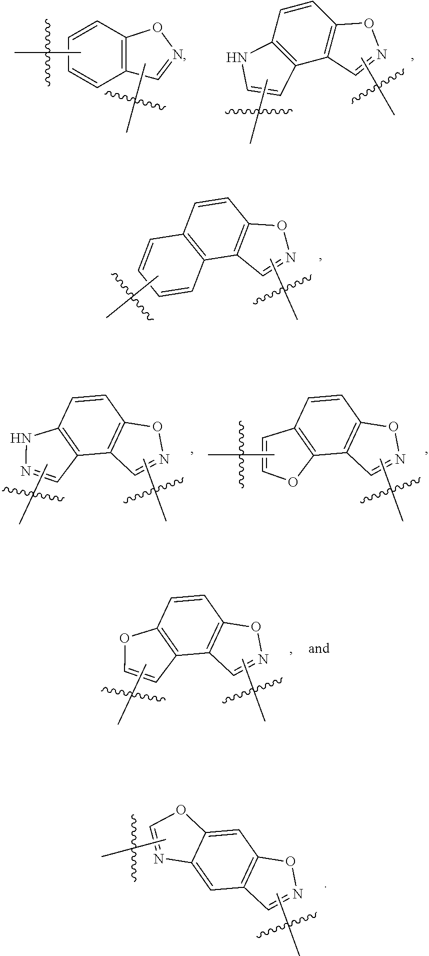

In some specific embodiments, the structural moiety

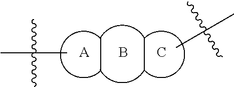

is selected from the group consisting of

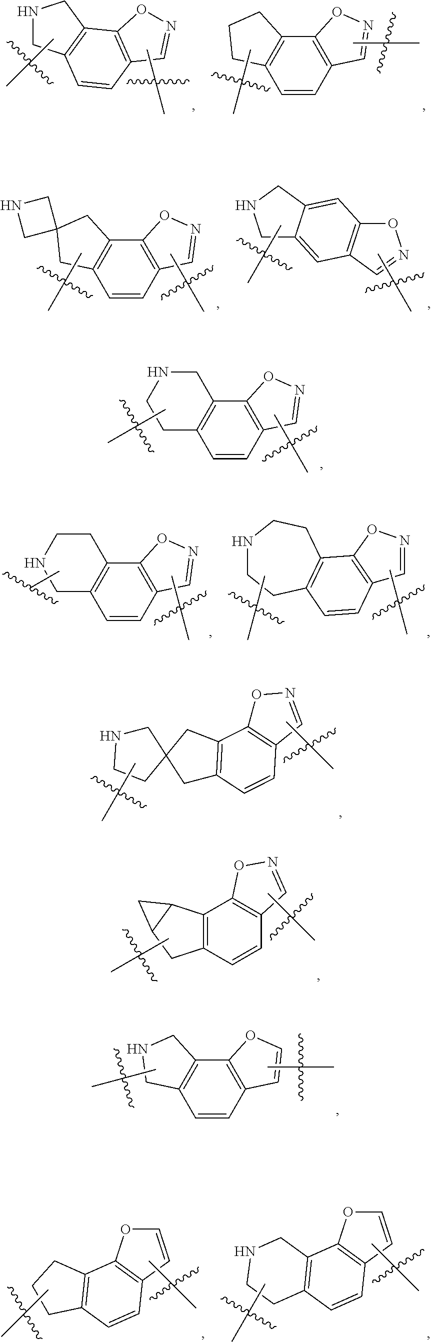

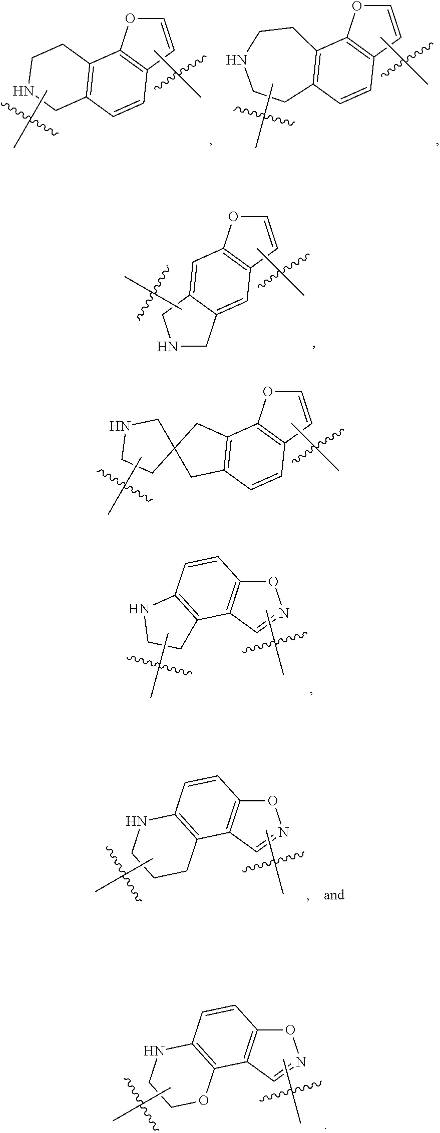

In some embodiments, the structural moiety

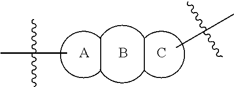

is selected from the group consisting of

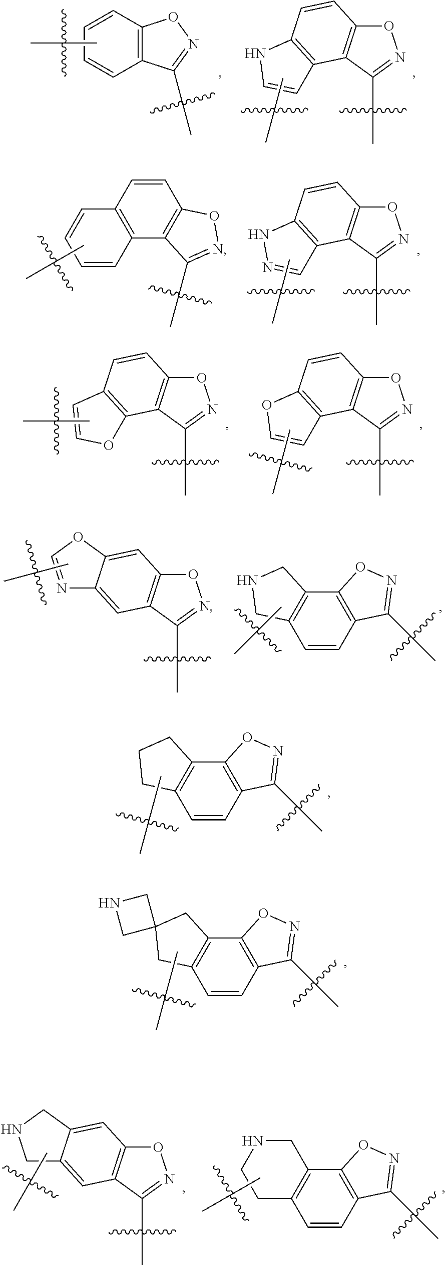

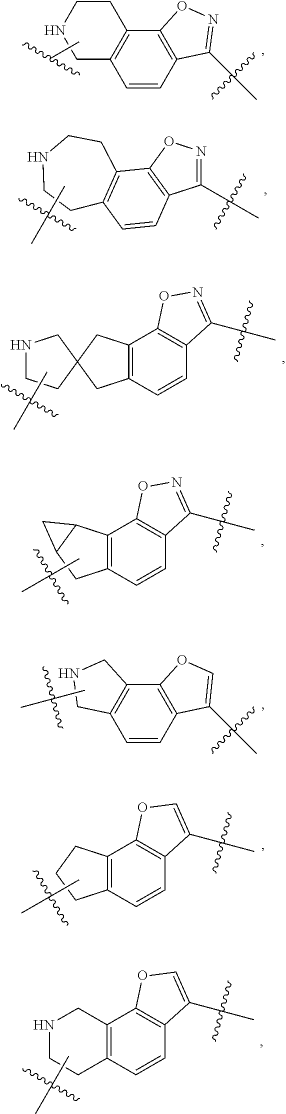

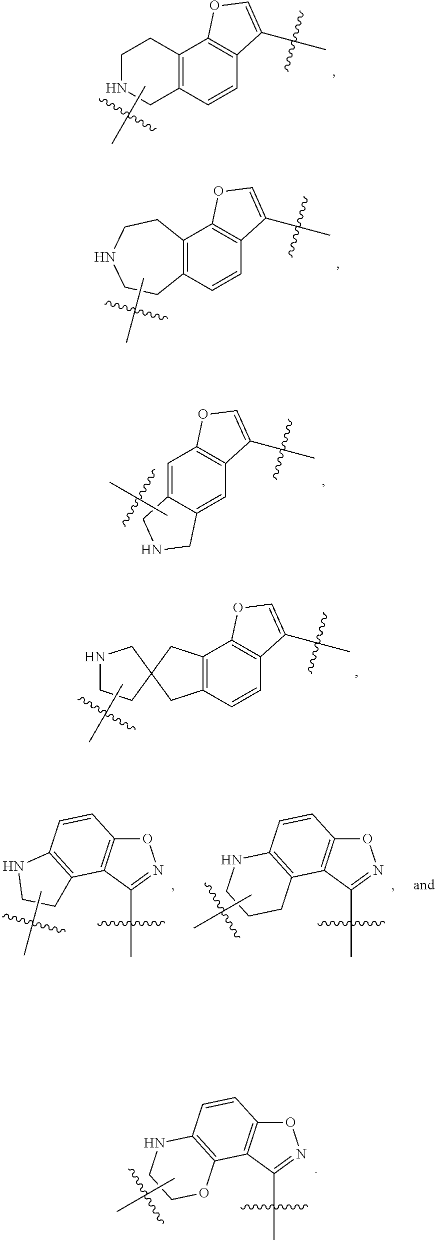

In some specific embodiments, the structural moiety

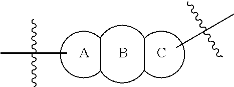

is selected from the group consisting of

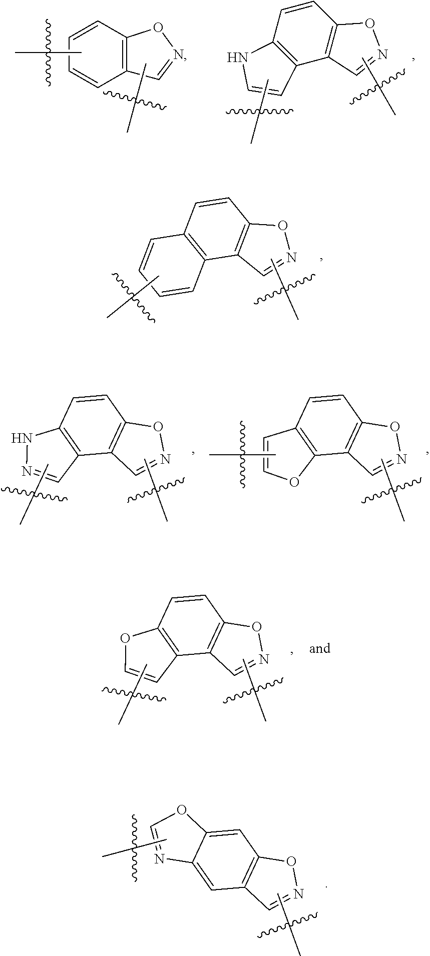

In some specific embodiments, the structural moiety

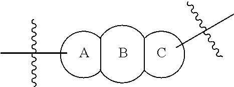

is selected from the group consisting of

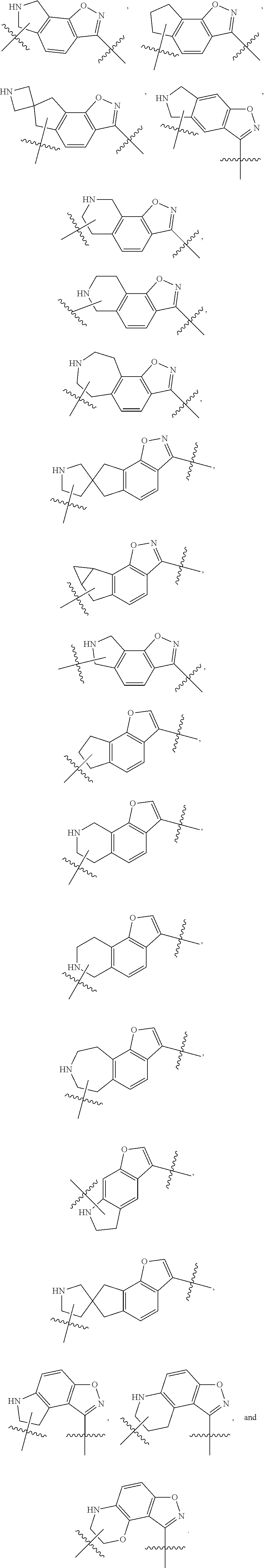

In some embodiments, the structural moiety

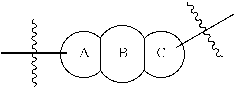

is selected from the group consisting of

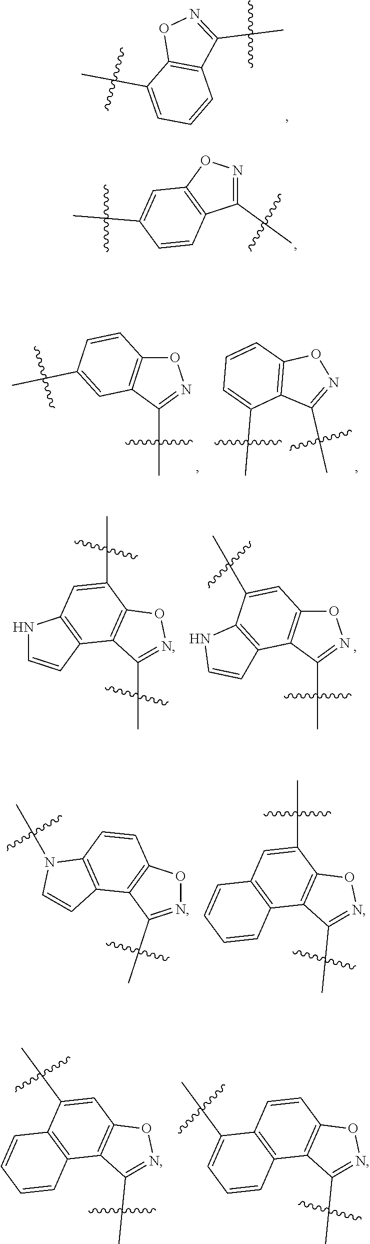

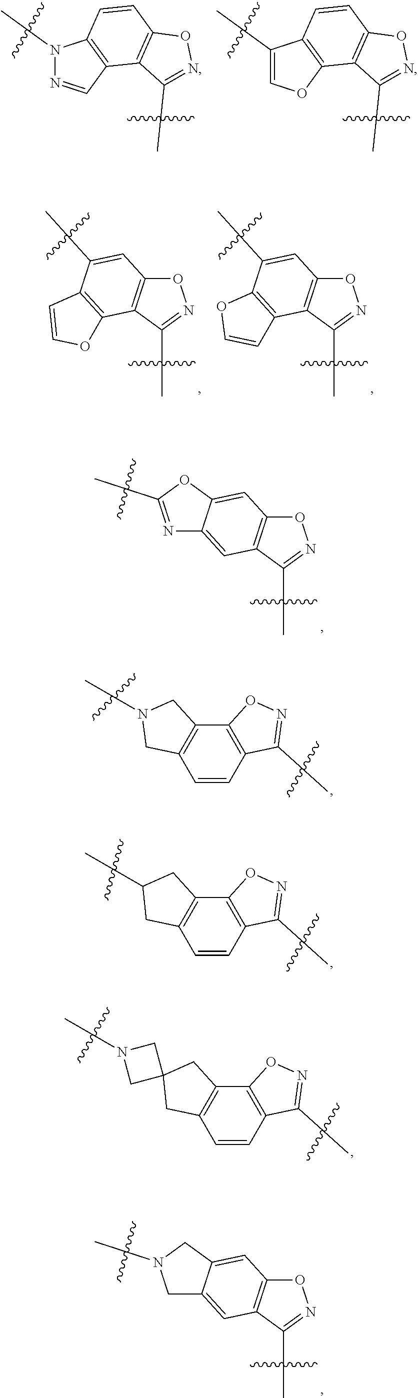

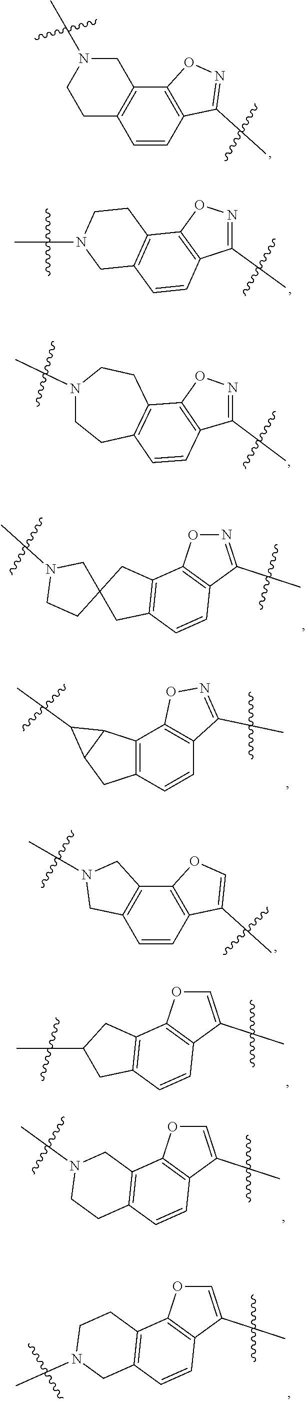

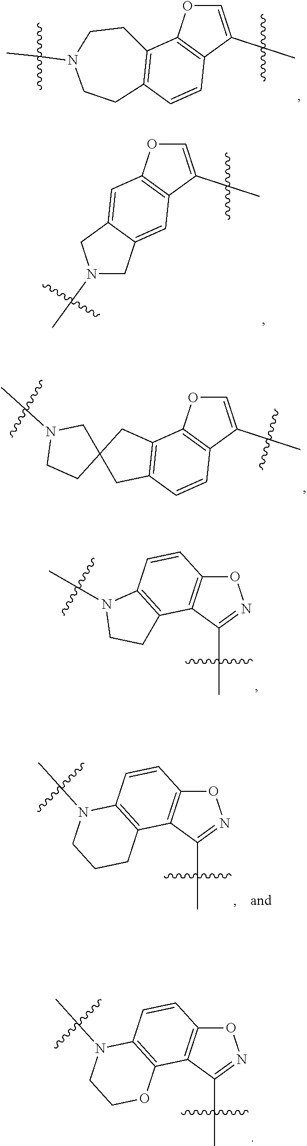

In some specific embodiments, the structural moiety

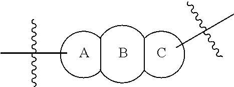

is selected from the group consisting of

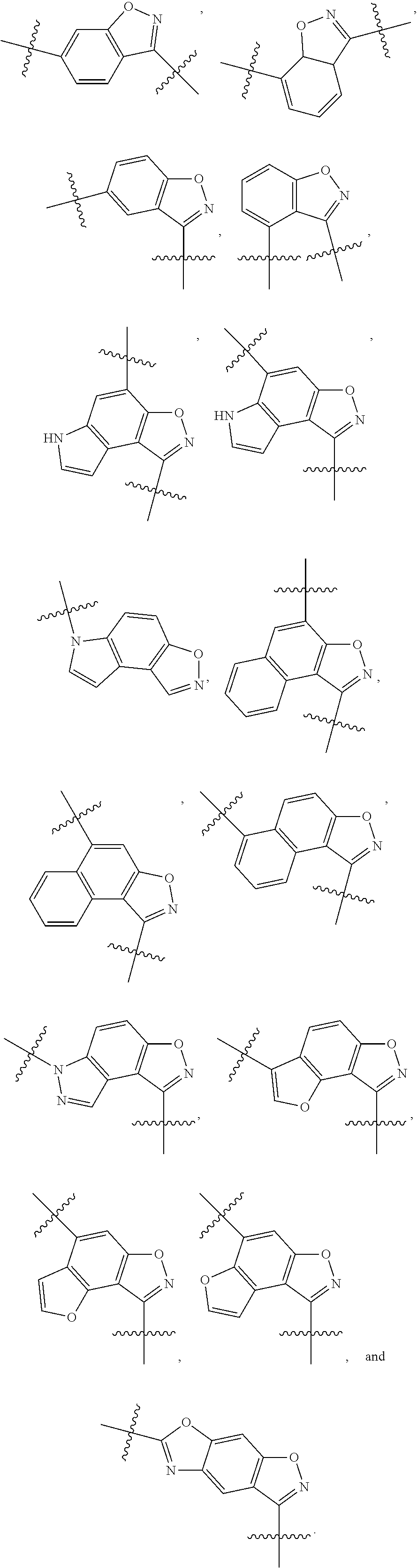

In some specific embodiments, the structural moiety

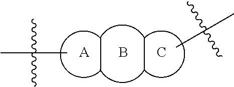

is selected from the group consisting of

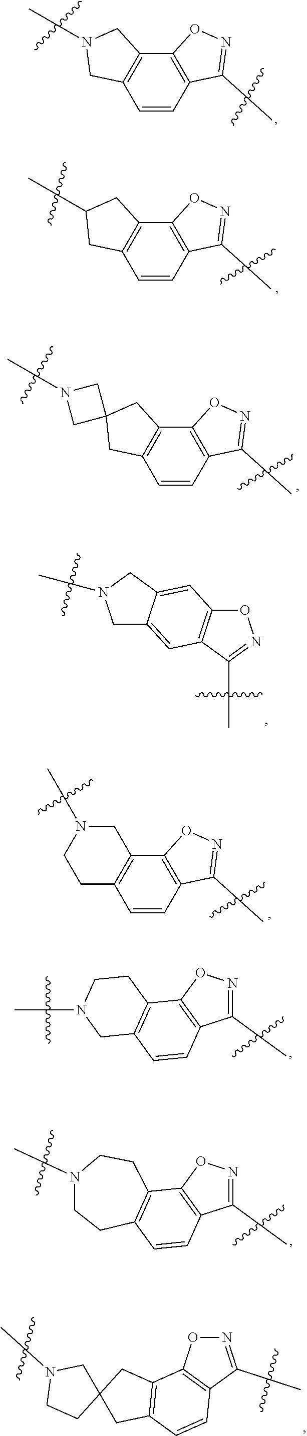

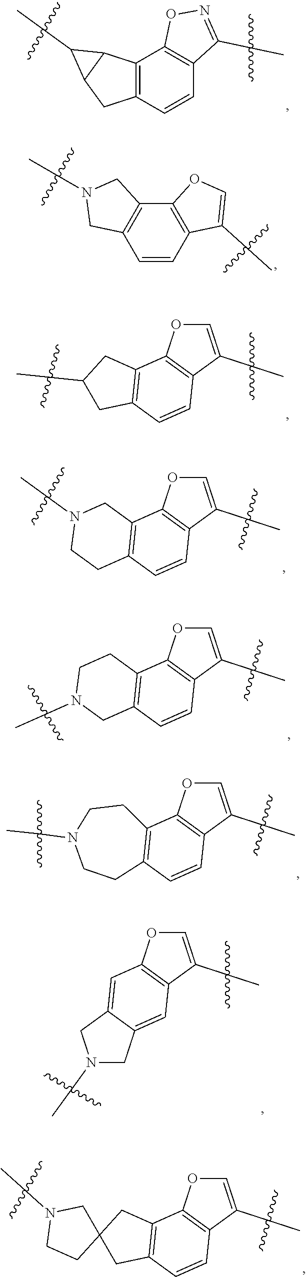

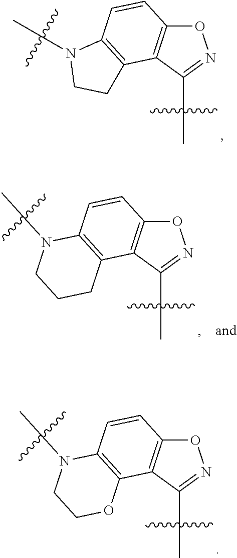 [SMILES: *c1noc2ccc3c(c12)CCN3* ; *c1noc2ccc3c(c12)CCCN3* ; *c1noc2ccc3c(c12)OCCN3*]

In some other embodiments, the structural moiety

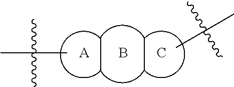

is selected from the group consisting of

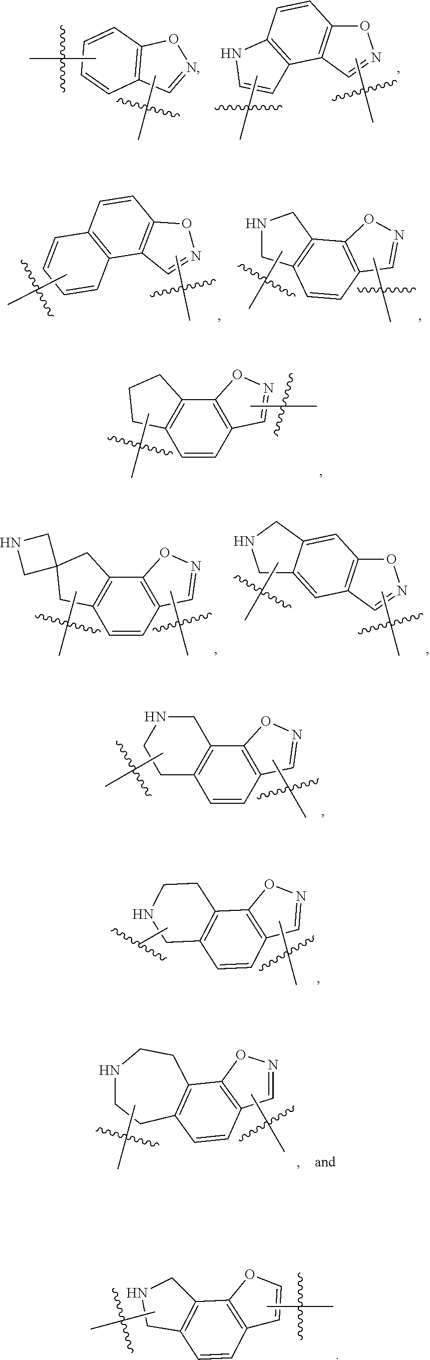

In some other embodiments, the structural moiety

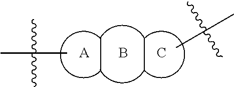

is selected from the group consisting of

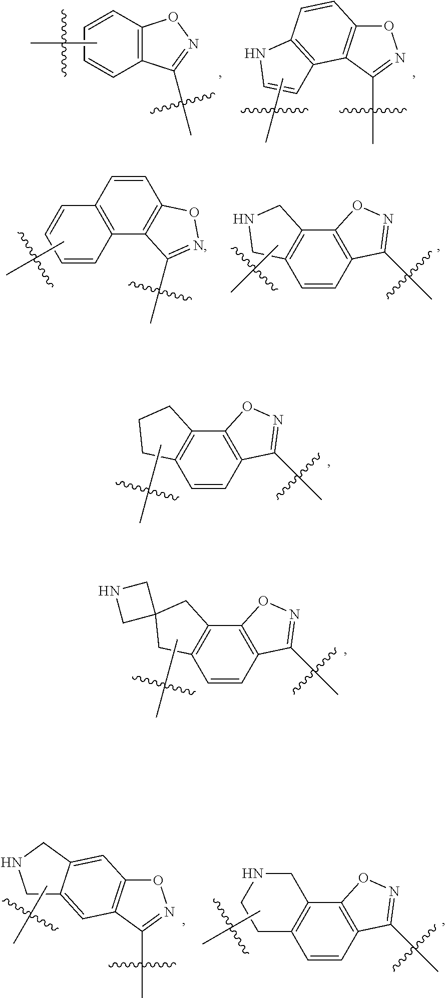

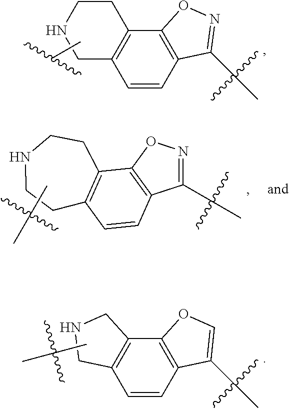

In some other embodiments, the structural moiety

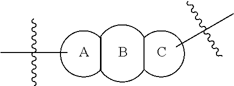

is selected from the group consisting of

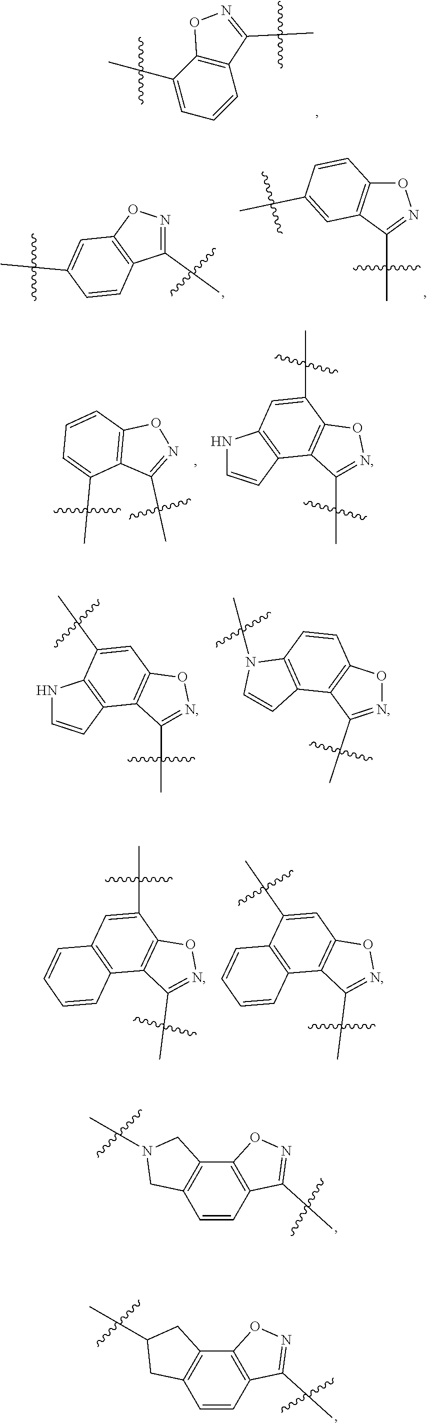

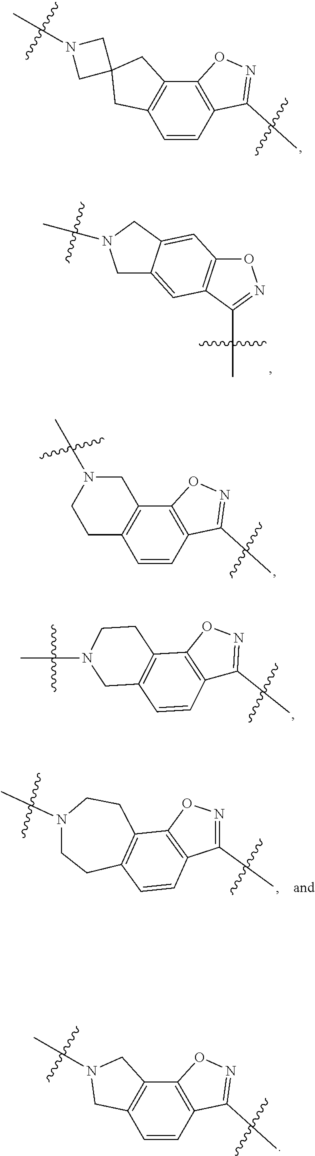 [SMILES: *c1noc2c3c(ccc12)CC1(C3)CN(*)C1 ; *c1noc2cc3c(cc12)CN(*)C3 ; *c1noc2c3c(ccc12)CCN(*)C3 ; *c1noc2c3c(ccc12)CN(*)CC3 ; *c1noc2c3c(ccc12)CCN(*)CC3 ; *c1noc2c3c(ccc12)CN(*)C3]

In some embodiments, the structural moiety

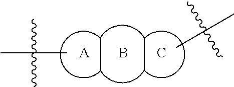

is selected from the group consisting of

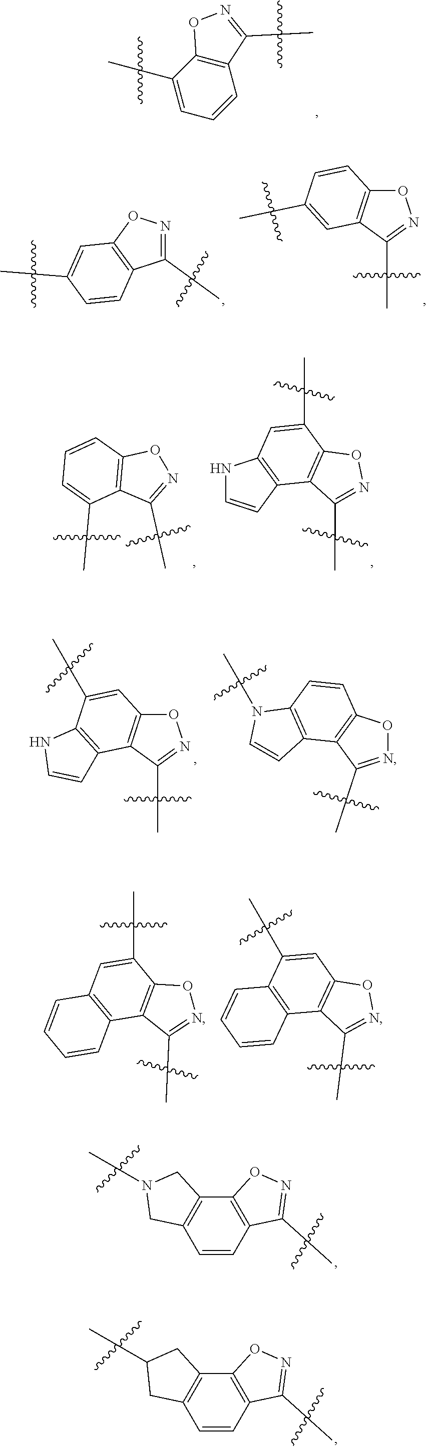

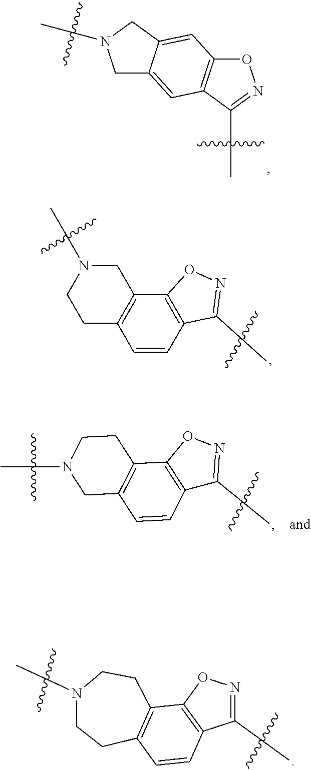 [SMILES: *c1noc2cc3c(cc12)CN(*)C3 ; *c1noc2c3c(ccc12)CCN(*)C3 ; *c1noc2c3c(ccc12)CN(*)CC3 ; *c1noc2c3c(ccc12)CCN(*)CC3]

In some embodiments, each R¹ is independently selected from the group consisting of halogen, —OH, —NW, —CN, C₁₋₃ alkyl, C₁₋₃ alkoxy, and C₁₋₃ haloalkyl.

In some embodiments, each R¹ is independently selected from the group consisting of fluorine, chlorine, bromine, —OH, —NH₂, and —CN. In some embodiments, each R¹ is independently selected from the group consisting of fluorine, chlorine, and bromine.

In some embodiments, each R¹ is independently selected from fluorine.

In some embodiments, n is selected from the group consisting of 0, 1, and 2. In some embodiments, n is selected from the group consisting of 0 and 1.

In some embodiments, the structural moiety

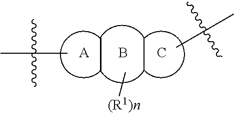

is selected from the group consisting of

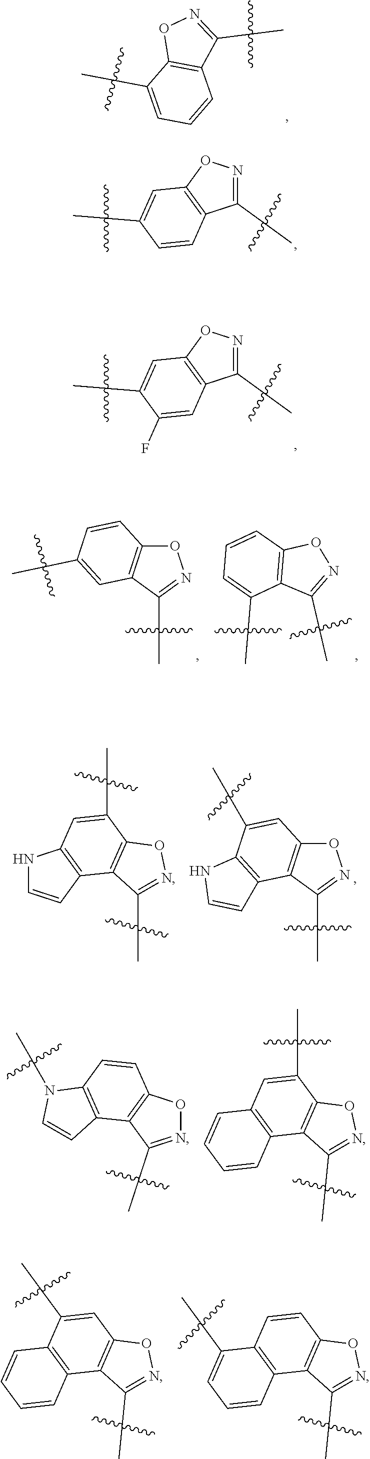

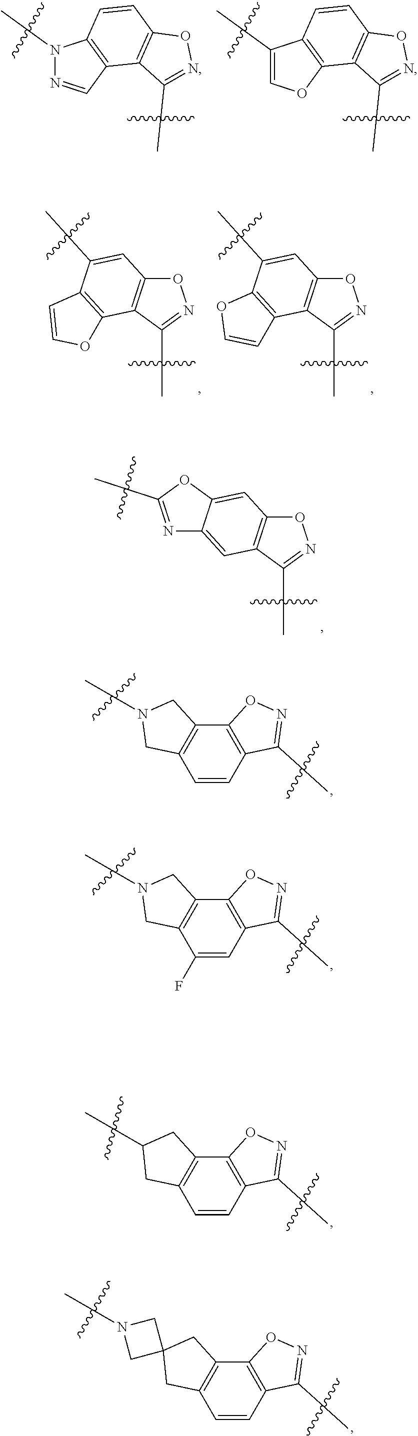

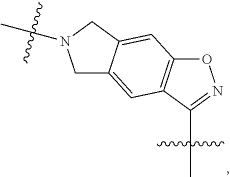 [SMILES: *c1noc2cc3c(cc12)CN(*)C3]

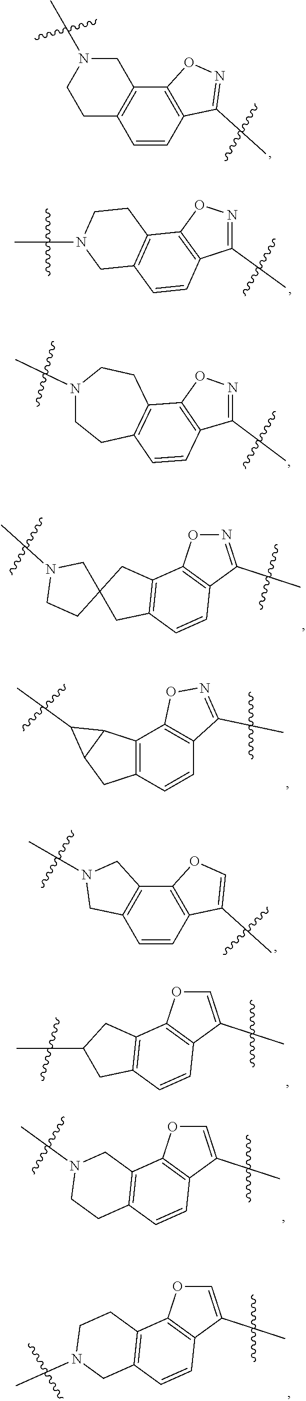

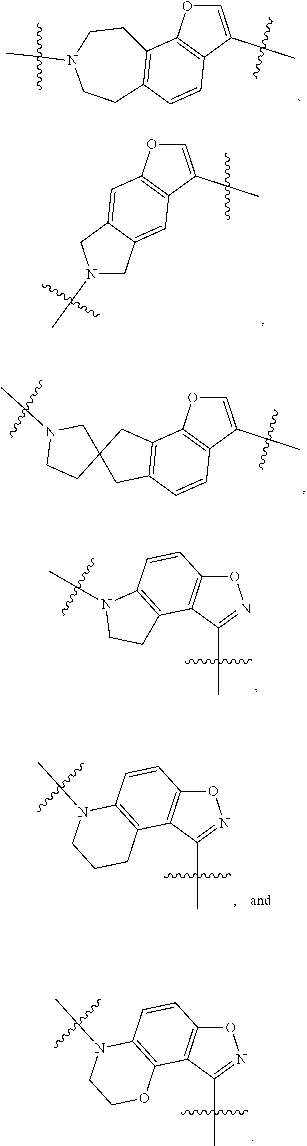

In some specific embodiments, the structural moiety

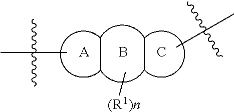

is selected from the group consisting of

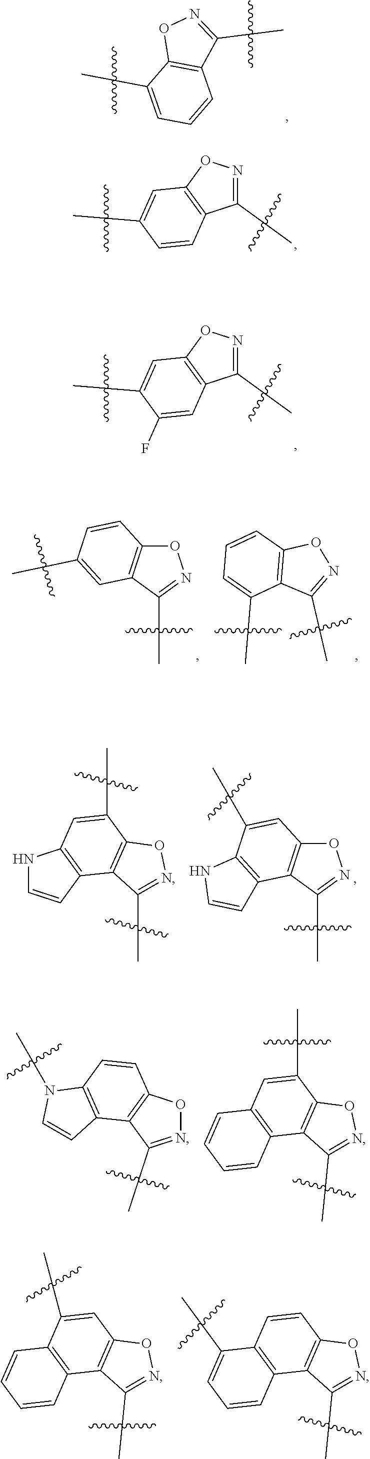

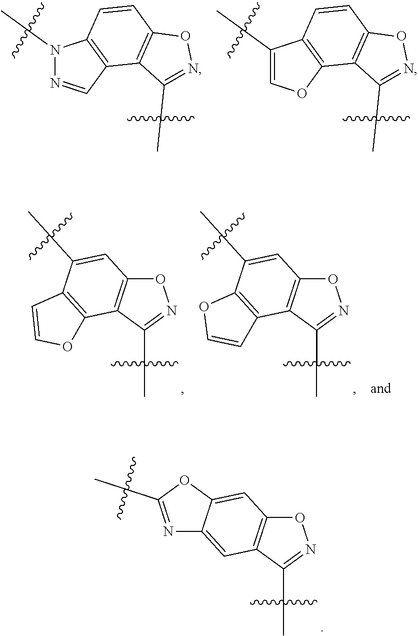

In some specific embodiments, the structural moiety

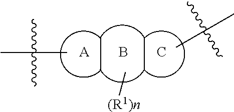

is selected from the group consisting of

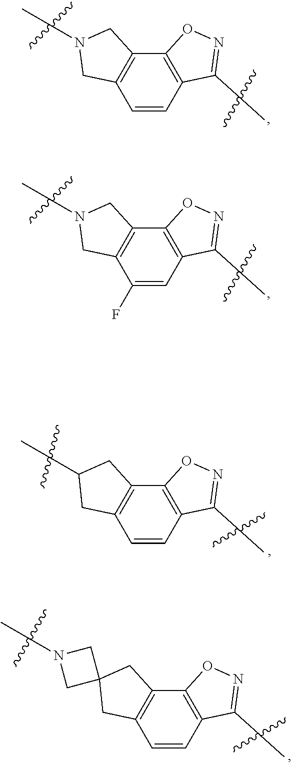 [SMILES: *c1noc2c3c(ccc12)CN(*)C3 ; *c1noc2c3c(c(F)cc12)CN(*)C3 ; *c1noc2c3c(ccc12)CC(*)C3 ; *c1noc2c3c(ccc12)CC1(C3)CN(*)C1]

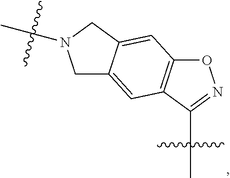 [SMILES: *c1noc2cc3c(cc12)CN(*)C3]

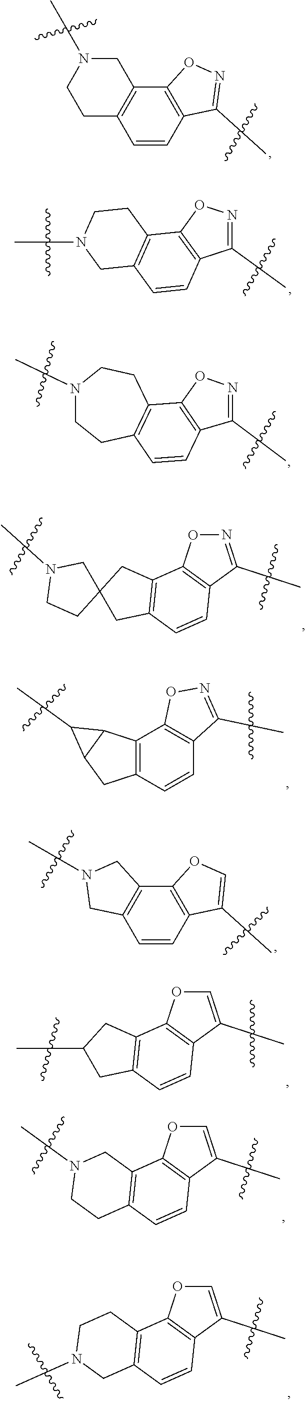

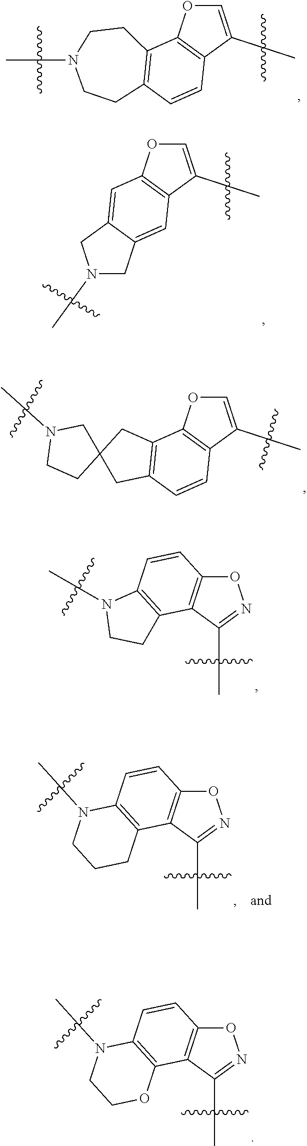

In some specific embodiments, the structural moiety

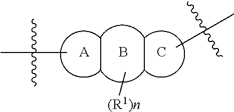

is selected from the group consisting of

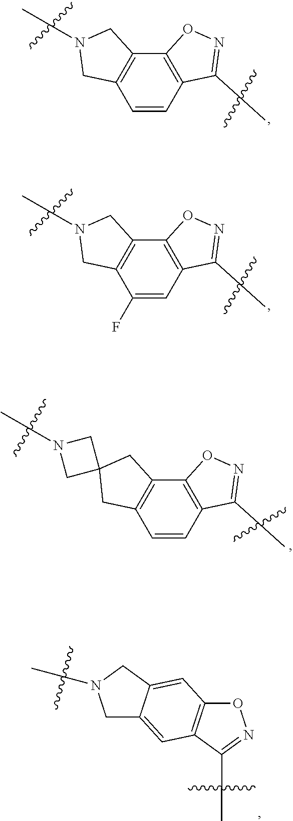 [SMILES: *c1noc2c3c(ccc12)CN(*)C3 ; *c1noc2c3c(c(F)cc12)CN(*)C3 ; *c1noc2c3c(ccc12)CC1(C3)CN(*)C1 ; *c1noc2cc3c(cc12)CN(*)C3]

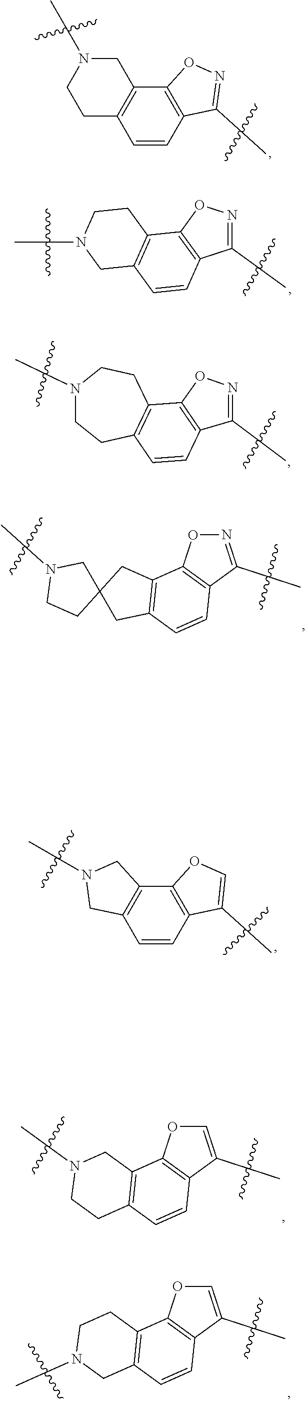

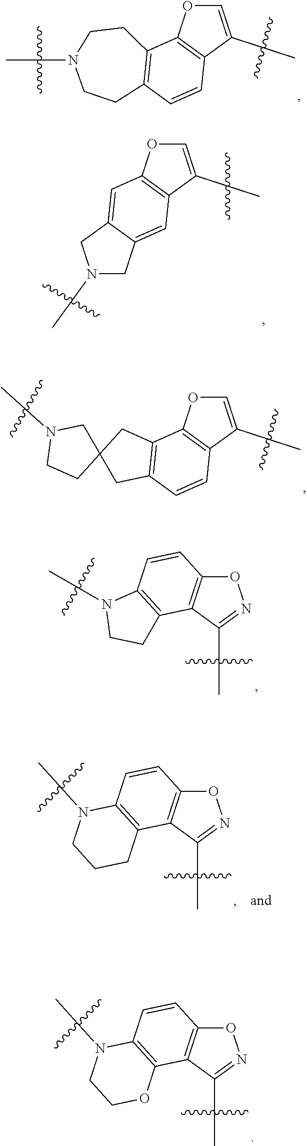

In some specific embodiments, the structural moiety

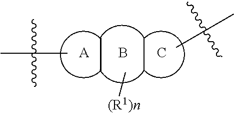

is selected from the group consisting of

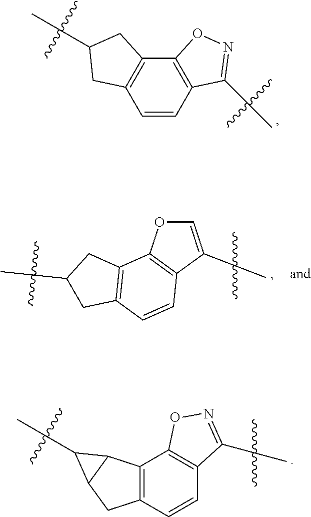 [SMILES: *c1noc2c3c(ccc12)CC(*)C3 ; *c1coc2c3c(ccc12)CC(*)C3 ; *c1noc2c3c(ccc12)CC1C(*)C31]

In some specific embodiments, the structural moiety

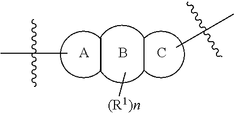

is selected from the group consisting of

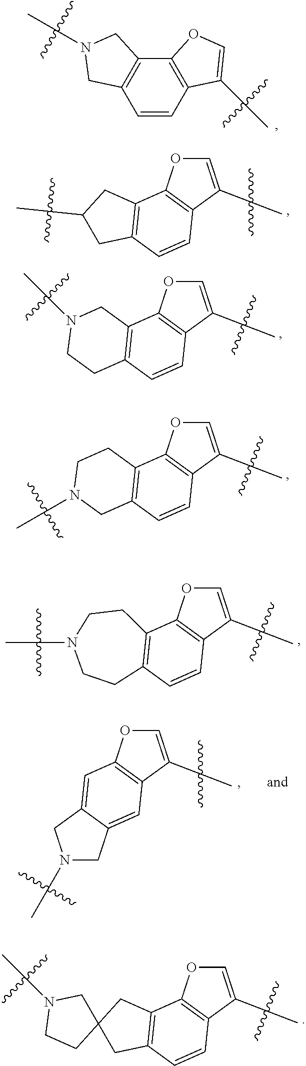

In some other embodiments, the structural moiety

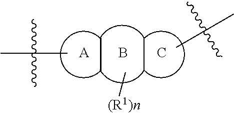

is selected from the group consisting of

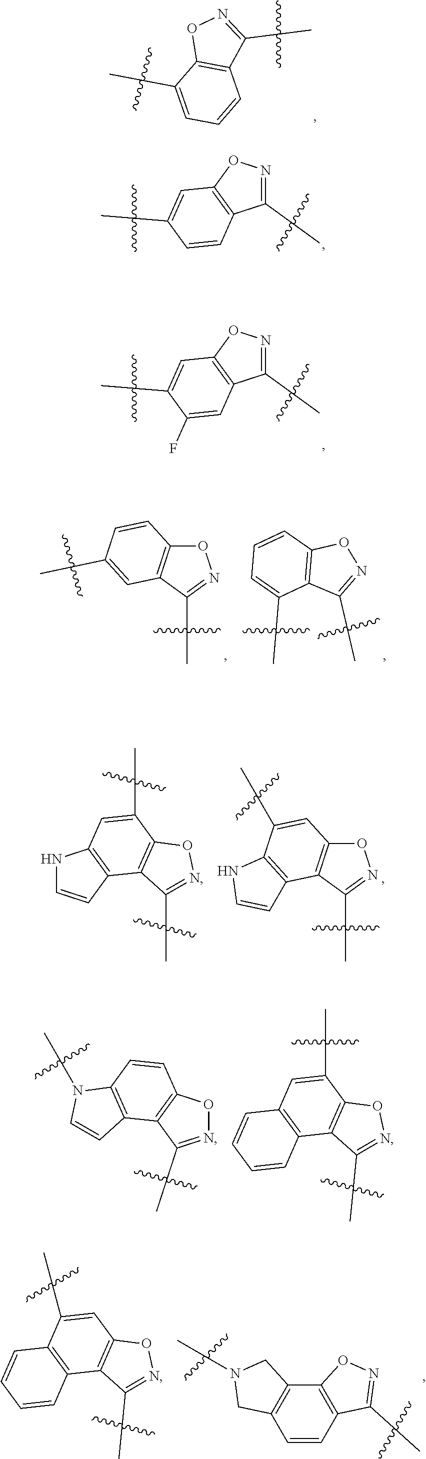

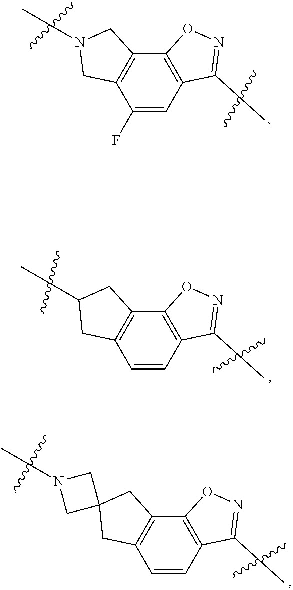 [SMILES: *c1noc2c3c(c(F)cc12)CN(*)C3 ; *c1noc2c3c(ccc12)CC(*)C3 ; *c1noc2c3c(ccc12)CC1(C3)CN(*)C1]

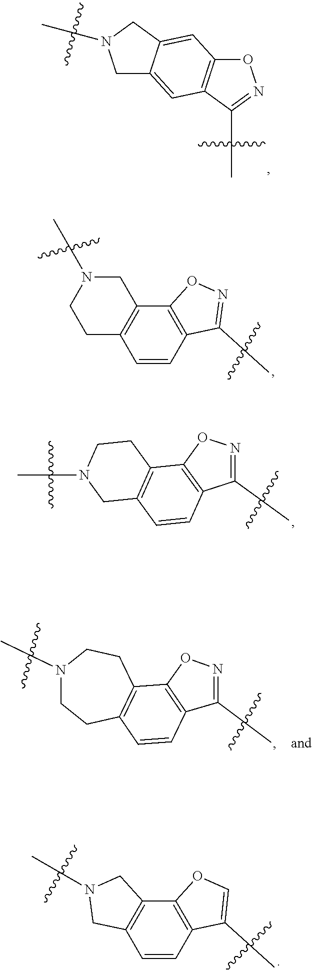 [SMILES: *c1noc2cc3c(cc12)CN(*)C3 ; *c1noc2c3c(ccc12)CCN(*)C3 ; *c1noc2c3c(ccc12)CN(*)CC3 ; *c1noc2c3c(ccc12)CCN(*)CC3 ; *c1coc2c3c(ccc12)CN(*)C3]

In some embodiments, the structural moiety

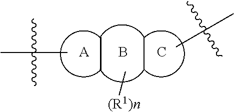

is selected from the group consisting of

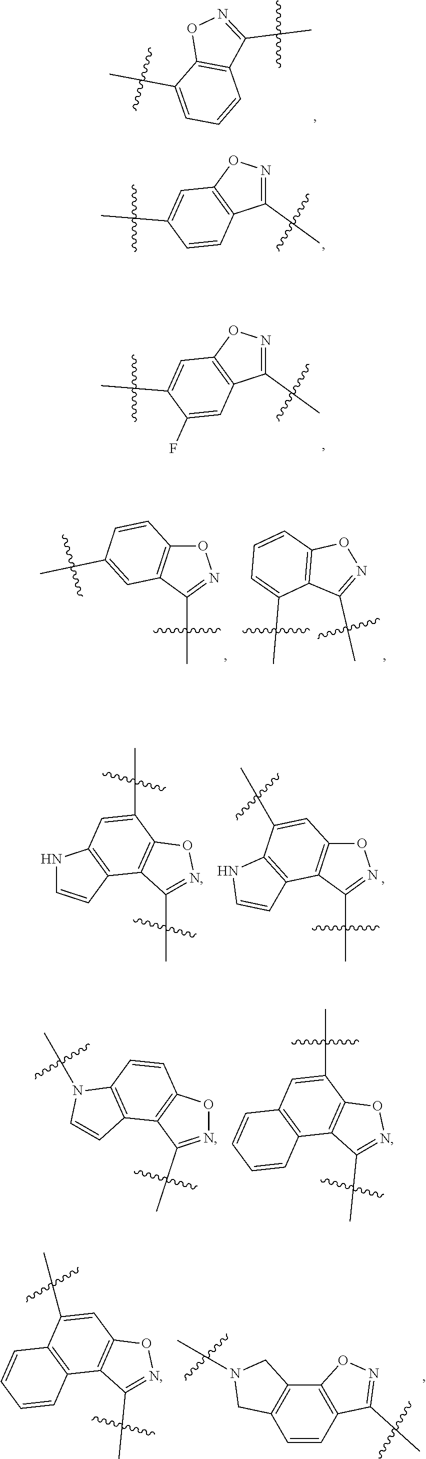

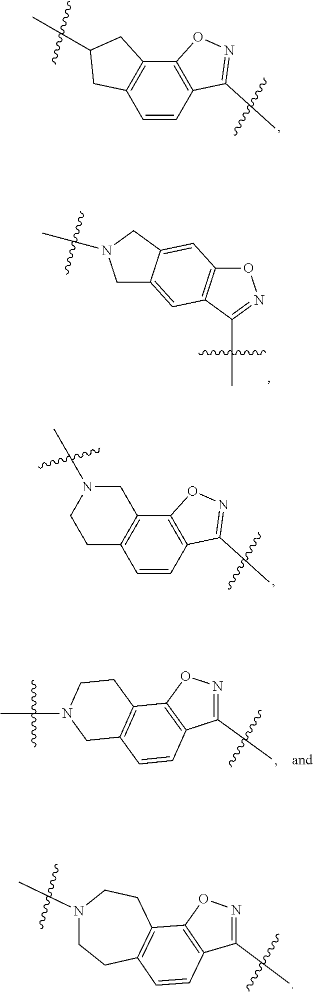 [SMILES: *c1noc2c3c(ccc12)CC(*)C3 ; *c1noc2cc3c(cc12)CN(*)C3 ; *c1noc2c3c(ccc12)CCN(*)C3 ; *c1noc2c3c(ccc12)CN(*)CC3 ; *c1noc2c3c(ccc12)CCN(*)CC3]

In some embodiments, PTM is

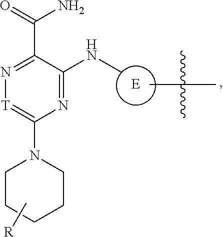

wherein
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl;
ring E is selected from the group consisting of phenyl, benzocycloalkenyl, and benzoheterocycloalkenyl.

In some specific embodiments, T is selected from CH.

In some specific embodiments, R is selected from the group consisting of hydrogen, imidazolidinonyl, and imidazolidinonyl substituted with C₁₋₄ alkyl; preferably, R is selected from the group consisting of hydrogen, imidazolidinonyl, 1-methyl-imidazolidinonyl, 1-ethyl-imidazolidinonyl, and 1-propyl-imidazolidinonyl.

In some specific embodiments, ring E is selected from the group consisting of phenyl, benzopiperidinyl, benzodihydropyrrolyl, spiro[benzopyran-piperidine], and benzodihydrooxazinopiperazine. In some specific embodiments, ring E is selected from phenyl.

In some specific embodiments, ring E is selected from phenyl, L is selected from -Cy¹-LNK-Cy²-, and X² is selected from CH, wherein Cy¹, LNK, and Cy² are as described herein.

In some embodiments, PTM is

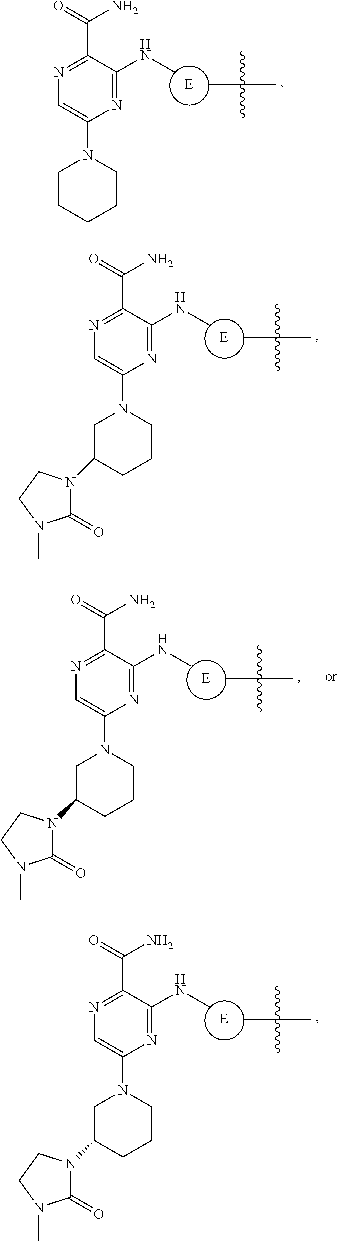

and ring E is as defined above. Preferably, PTM is

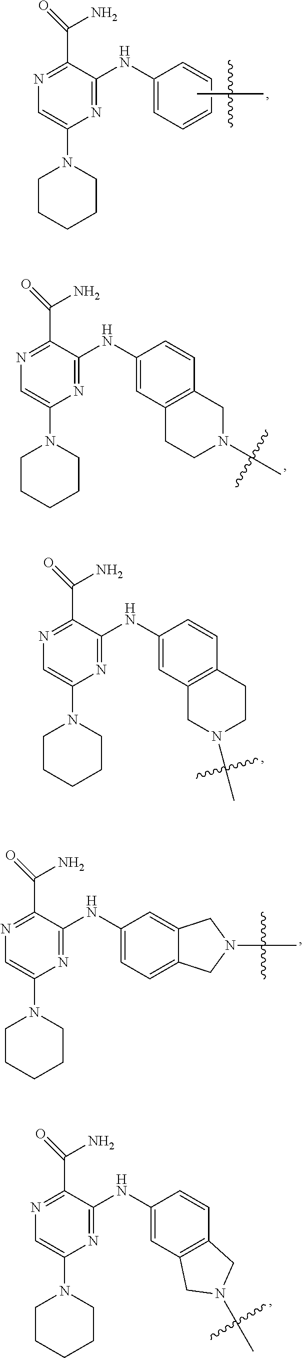

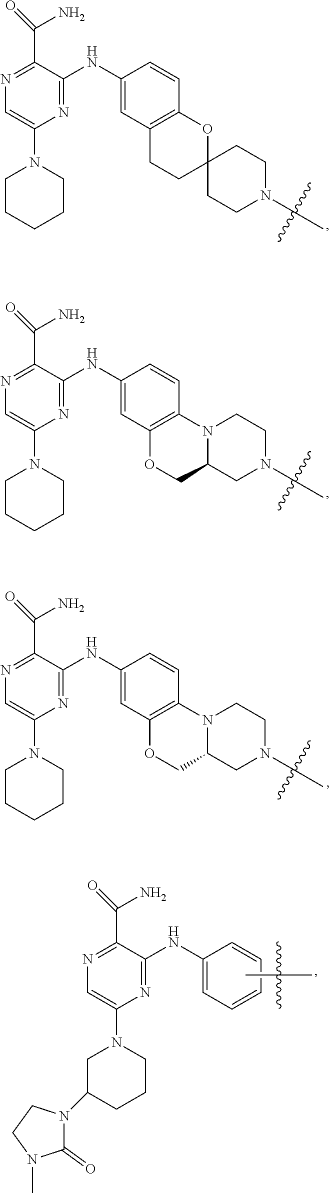

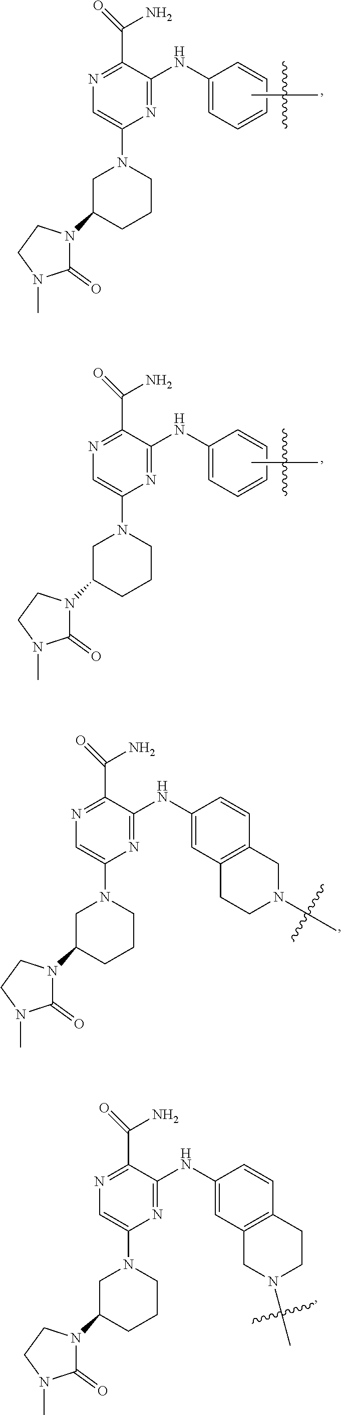

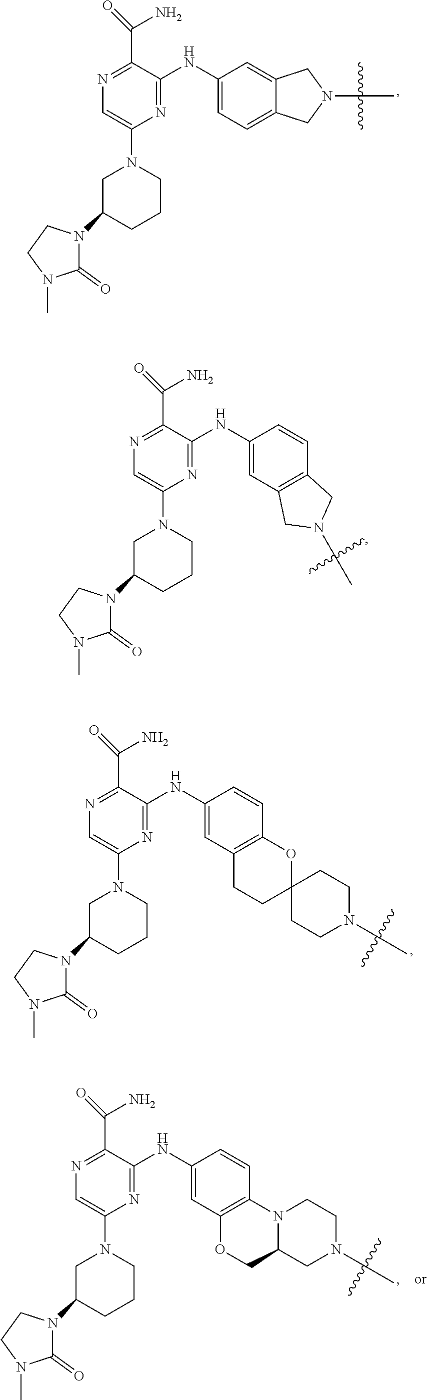 [SMILES: *N1Cc2ccc(Nc3nc(N4CCC[C@@H](N5CCN(C)C5=O)C4)cnc3C(N)=O)cc2C1 ; *N1Cc2ccc(Nc3nc(N4CCC[C@@H](N5CCN(C)C5=O)C4)cnc3C(N)=O)cc2C1 ; *N1CCC2(CCc3cc(Nc4nc(N5CCC[C@@H](N6CCN(C)C6=O)C5)cnc4C(N)=O)ccc3O2)CC1 ; *N1CCN2c3ccc(Nc4nc(N5CCC[C@@H](N6CCN(C)C6=O)C5)cnc4C(N)=O)cc3OC[C@@H]2C1]

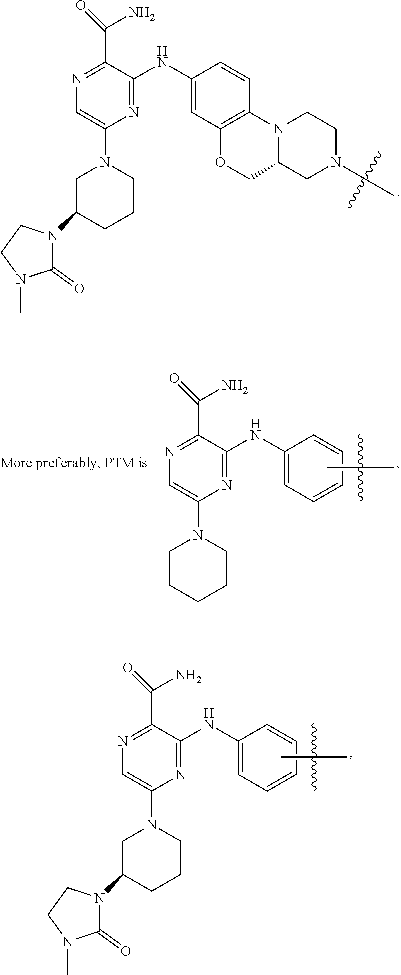

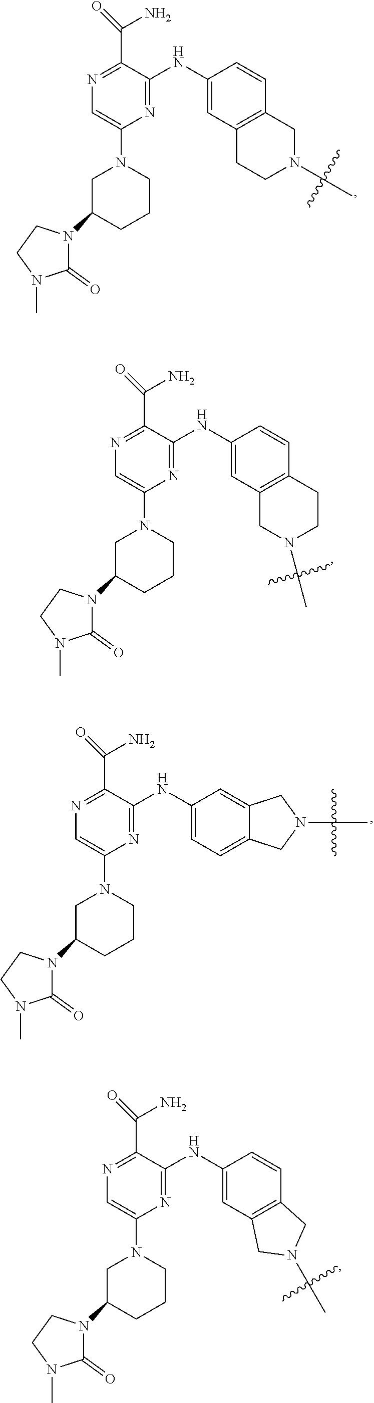 [SMILES: *N1CCc2cc(Nc3nc(N4CCC[C@@H](N5CCN(C)C5=O)C4)cnc3C(N)=O)ccc2C1 ; *N1CCc2ccc(Nc3nc(N4CCC[C@@H](N5CCN(C)C5=O)C4)cnc3C(N)=O)cc2C1 ; *N1Cc2ccc(Nc3nc(N4CCC[C@@H](N5CCN(C)C5=O)C4)cnc3C(N)=O)cc2C1 ; *N1Cc2ccc(Nc3nc(N4CCC[C@@H](N5CCN(C)C5=O)C4)cnc3C(N)=O)cc2C1]

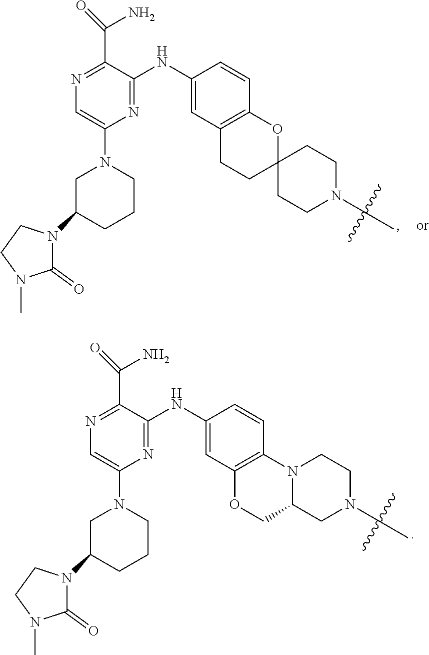 [SMILES: *N1CCC2(CCc3cc(Nc4nc(N5CCC[C@@H](N6CCN(C)C6=O)C5)cnc4C(N)=O)ccc3O2)CC1 ; *N1CCN2c3ccc(Nc4nc(N5CCC[C@@H](N6CCN(C)C6=O)C5)cnc4C(N)=O)cc3OC[C@H]2C1]

In some embodiments, the structural moiety -Cy¹-LNK-Cy²- is selected from the group consisting of -Cy¹-, -Cy¹-LNK-, -Cy¹-Cy²-, -Cy¹-LNK-Cy²-, -Cy²-, and -LNK-Cy²-. In some embodiments, the structural moiety -Cy¹-LNK-Cy²- is selected from the group consisting of -Cy¹-, -Cy¹-Cy²-, and -Cy²-. In some specific embodiments, the structural moiety -Cy¹-LNK-Cy²- is selected from the group consisting of -Cy¹-LNK-, -Cy¹-LNK-Cy²-, and -LNK-Cy²-. In some specific embodiments, the structural moiety -Cy¹-LNK-Cy²- is selected from -Cy¹-LNK-. In some specific embodiments, the structural moiety -Cy¹-LNK-Cy²- is selected from -Cy¹-LNK-Cy²-. In some specific embodiments, the structural moiety -Cy¹-LNK-Cy²- is selected from -LNK-Cy²-.

In some other embodiments, the structural moiety -Cy¹-LNK-Cy²- is selected from the group consisting of -Cy¹-, -Cy¹-LNK, -Cy¹-Cy²-, and -Cy¹-LNK-Cy²-.

In some embodiments, Cy¹ is selected from the group consisting of a bond and the following groups optionally substituted with one or more R^{a}: C₄₋₁₁ cycloalkyl or 4- to 11-membered heterocycloalkyl.

In some embodiments, Cy¹ is selected from the group consisting of a bond and the following groups optionally substituted with one or more R^{a}: C₆₋₉ cycloalkyl or 4- to 11-membered heterocycloalkyl. In some embodiments, Cy¹ is selected from the group consisting of a bond and the following groups optionally substituted with one or more R^{a}: C₆₋₉ cycloalkyl (e.g., C₆ cycloalkyl and C₉ cycloalkyl) and 5- to 11-membered heterocycloalkyl containing 1-3 heteroatoms selected from the group consisting of N, O, and S (e.g., 1-3 or 1-2 heteroatoms selected from the group consisting of N and O).

In some embodiments, Cy¹ is selected from the group consisting of a bond and the following groups optionally substituted with one or more R^{a}: C₆ cycloalkyl, C₉ cycloalkyl, and 4-, 5-, 6-, 7-, 8-, 9-, 10-, or 11-membered heterocycloalkyl.

In some embodiments, Cy¹ is selected from the group consisting of a bond and the following groups optionally substituted with one or more R^{a}: C₆ cycloalkyl, C₉ cycloalkyl, and 4-, 6-, or 8- to 11-membered heterocycloalkyl.

In some specific embodiments, Cy¹ is selected from a bond. In some specific embodiments, Cy¹ is selected from the group consisting of C₆ cycloalkyl and C₉ cycloalkyl, wherein the cycloalkyl is optionally substituted with one or more R^{a}. In some specific embodiments, Cy¹ is selected from the group consisting of 4-, 6-, and 8- to 11-membered heterocycloalkyl, wherein the heterocycloalkyl is optionally substituted with one or more R^{a}. In some specific embodiments, Cy¹ is selected from the group consisting of 4- and 6-membered heterocycloalkyl, wherein the heterocycloalkyl is optionally substituted with one or more R^{a}. In some specific embodiments, Cy¹ is selected from the group consisting of 8- to 11-membered heterocycloalkyl, wherein the heterocycloalkyl is optionally substituted with one or more R^{a}. In some specific embodiments, Cy¹ is selected from the group consisting of 8-, 9-, 10-, and 11-membered heterocycloalkyl, wherein the heterocycloalkyl is optionally substituted with one or more R^{a}.

In some embodiments, Cy¹ is selected from the group consisting of a bond and the following groups optionally substituted with one or more R^{a}: cyclohexyl, spirononanyl, azetidinyl, octahydrocyclopentapyrrolyl, piperidinyl, monoazaspirononanyl, diazaspirononanyl, azabicyclononanyl (e.g., monoazabicyclononanyl), monoazaspiroundecanyl, or diazaspiroundecanyl.

In some specific embodiments, Cy¹ is selected from the group consisting of cyclohexyl and spirononanyl. In some specific embodiments, Cy¹ is selected from the group consisting of azetidinyl and piperidinyl. In some specific embodiments, Cy¹ is selected from the group consisting of octahydrocyclopentapyrrolyl, monoazaspirononanyl, diazaspirononanyl, azabicyclononanyl, monoazaspiroundecanyl, and diazaspiroundecanyl. In some specific embodiments, Cy¹ is selected from the group consisting of a bond, piperidinyl, cyclohexyl, spirononanyl, monoazaspirononanyl, octahydrocyclopentapyrrolyl, monoazaspiroundecanyl, monooxamonoazaspiroundecanyl, and azabicyclononanyl.

In some embodiments, Cy¹ is selected from the group consisting of a bond and the following groups optionally substituted with one or more R^{a}:

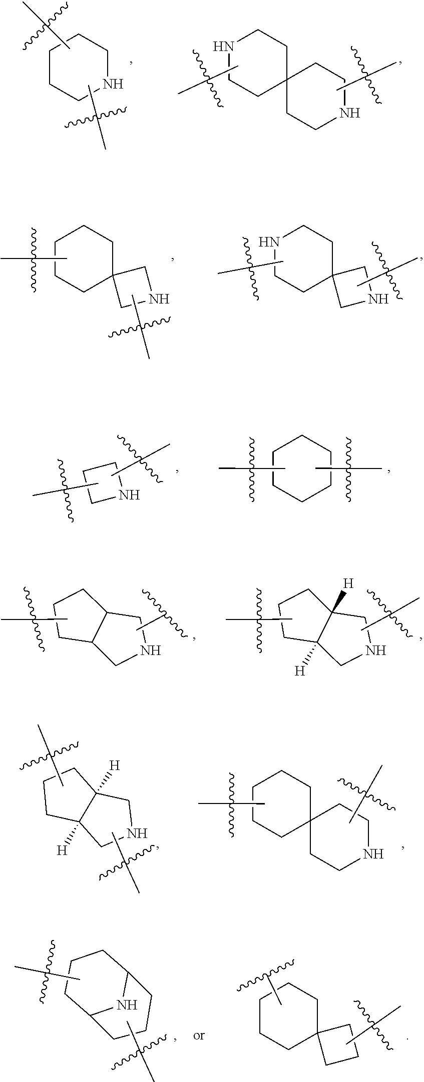

In some embodiments, Cy¹ is selected from the group consisting of a bond and the following groups optionally substituted with one or more R^{a}:

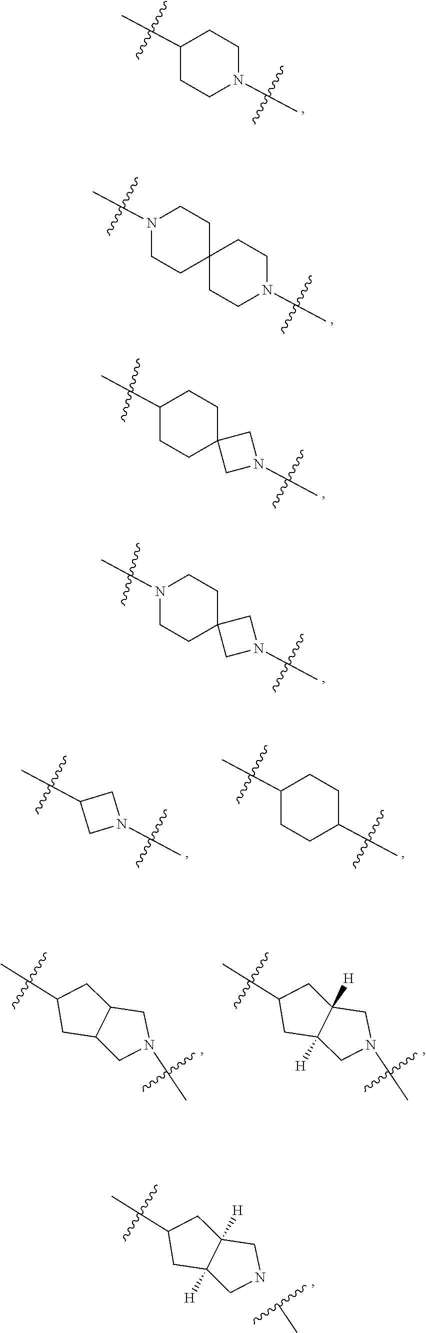

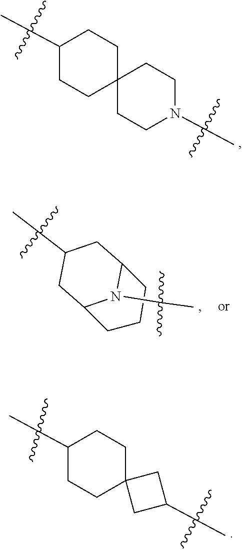 [SMILES: *C1CCC2(CC1)CCN(*)CC2 ; *C1CC2CCCC(C1)N2* ; *C1CCC2(CC1)CC(*)C2]

In some specific embodiments, Cy¹ is selected from the group consisting of

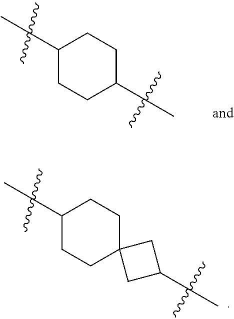 [SMILES: *C1CCC(*)CC1 ; *C1CCC2(CC1)CC(*)C2]

In some specific embodiments, Cy¹ is selected from the group consisting of

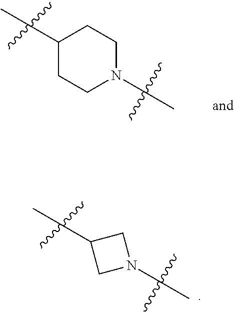 [SMILES: *C1CCN(*)CC1 ; *C1CN(*)C1]

In some specific embodiments, Cy¹ is selected from the group consisting of

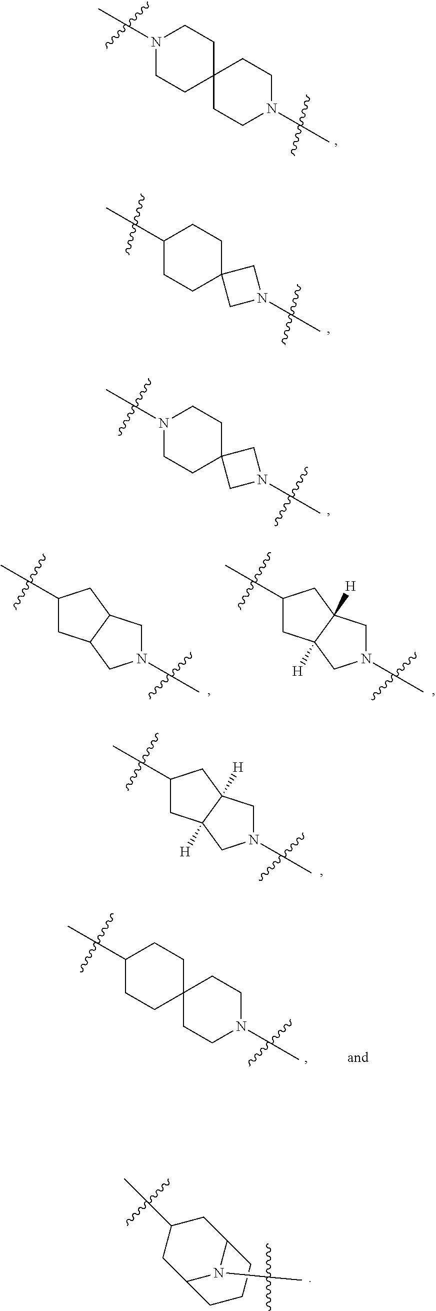

In some specific embodiments, Cy¹ is selected from the group consisting of a bond,

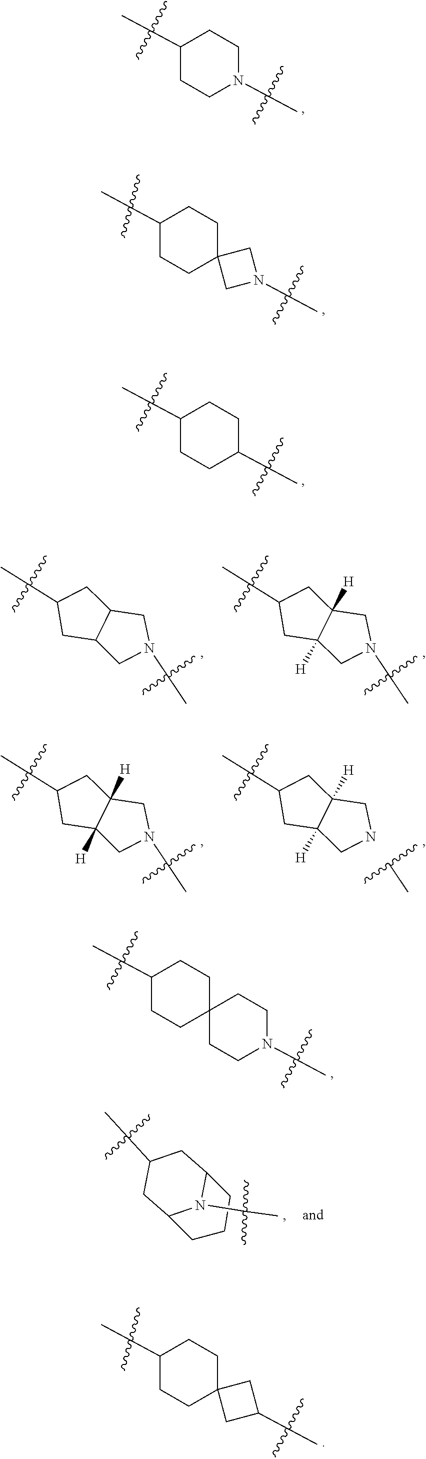

In some other embodiments, Cy¹ is selected from the group consisting of 4- to 11-membered heterocycloalkyl optionally substituted with one or more R^{a}.

In some other embodiments, Cy¹ is selected from the group consisting of 5-, 6-, 7-, 8-, 9-, 10-, and 11-membered heterocycloalkyl optionally substituted with one or more R^{a}.

In some other embodiments, Cy¹ is selected from the group consisting of 6-, and 9- to 11-membered heterocycloalkyl optionally substituted with one or more R^{a}.

In some other embodiments, Cy¹ is selected from the group consisting of 6-, 9-, and 11-membered heterocycloalkyl optionally substituted with one or more R^{a}.

In some other embodiments, Cy¹ is selected from the group consisting of piperidinyl, monoazaspirononane, diazaspirononane, and diazaspiroundecane optionally substituted with one or more R^{a}.

In some other embodiments, Cy¹ is selected from the group consisting of

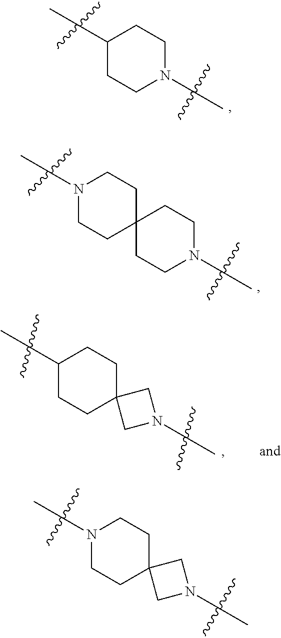 [SMILES: *C1CCN(*)CC1 ; *N1CCC2(CC1)CCN(*)CC2 ; *C1CCC2(CC1)CN(*)C2 ; *N1CCC2(CC1)CN(*)C2]

optionally substituted with one or more R^{a}.

In some embodiments, LNK is selected from the group consisting of a bond, C₁₋₆ alkylene, and C₁₋₆ heteroalkylene.

In some embodiments, LNK is selected from the group consisting of a bond and C₁₋₄ alkylene.

In some embodiments, LNK is selected from the group consisting of a bond and C₁₋₃ alkylene.

In some embodiments, LNK is selected from the group consisting of a bond and —CH₂—. In some specific embodiments, LNK is selected from a bond. In some specific embodiments, LNK is selected from —CH₂—.

In some embodiments, Cy² is absent or selected from the group consisting of C₄₋₁₁ cycloalkyl and 4- to 11-membered heterocycloalkyl, wherein the cycloalkyl or heterocycloalkyl is optionally substituted with one or more R^{b}. In some specific embodiments, Cy² is absent. In some specific embodiments, Cy² is selected from the group consisting of C₄₋₁₁ cycloalkyl and 4- to 11-membered heterocycloalkyl, wherein the cycloalkyl or heterocycloalkyl is optionally substituted with one or more R^{b}.

In some embodiments, Cy² is absent or selected from the group consisting of C₄₋₆ cycloalkyl and 4- to 6-membered heterocycloalkyl, wherein the cycloalkyl or heterocycloalkyl is optionally substituted with one or more R^{b}. In some specific embodiments, Cy² is selected from the group consisting of C₄₋₆ cycloalkyl and 4- to 6-membered heterocycloalkyl, wherein the cycloalkyl or heterocycloalkyl is optionally substituted with one or more R^{b}. In some embodiments, Cy² is absent or selected from the group consisting of C₄₋₆ cycloalkyl and 4- to 6-membered heterocycloalkyl, wherein the cycloalkyl or heterocycloalkyl is optionally substituted with one or more R^{b}, wherein the heterocycloalkyl comprises 1-3 (e.g., 1-2) heteroatoms selected from the group consisting of N, O, and S (e.g., N or O).

In some embodiments, Cy² is absent or selected from the group consisting of cyclobutyl, cyclopentyl, cyclohexyl, azetidinyl, pyrrolidinyl, and piperidinyl, wherein the cyclobutyl, cyclopentyl, azetidinyl, pyrrolidinyl, or piperidinyl is optionally substituted with one or more R^{b}.

In some embodiments, Cy² is absent or selected from the group consisting of

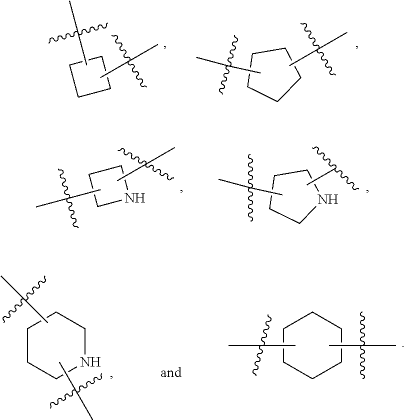

In some embodiments, Cy² is absent or selected from the group consisting of

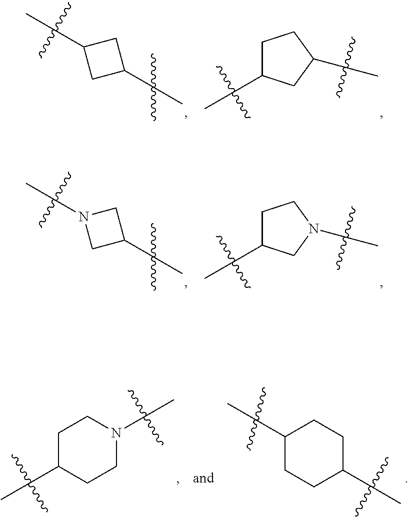 [SMILES: *C1CC(*)C1 ; *C1CCC(*)C1 ; *C1CN(*)C1 ; *C1CCN(*)C1 ; *C1CCN(*)CC1 ; *C1CCC(*)CC1]

In some other embodiments, Cy² is absent or selected from the group consisting of cyclobutyl, cyclopentyl, azetidinyl, pyrrolidinyl, and piperidinyl, wherein the cyclobutyl, cyclopentyl, azetidinyl, pyrrolidinyl, or piperidinyl is optionally substituted with one or more R^{b}.

In some other embodiments, Cy² is selected from the group consisting of

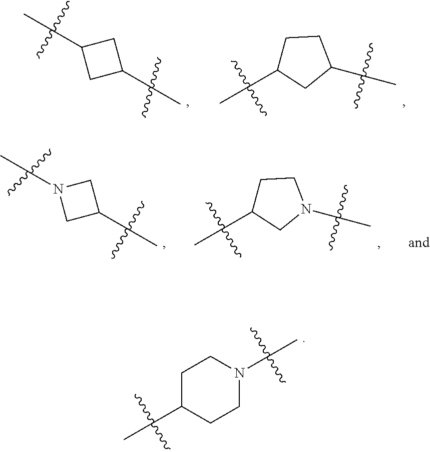 [SMILES: *C1CC(*)C1 ; *C1CCC(*)C1 ; *C1CN(*)C1 ; *C1CCN(*)C1 ; *C1CCN(*)CC1]

In some embodiments, R^{a} and R^{b} are each independently selected from the group consisting of halogen, —OH, —NH₂, —CN, C₁₋₃ alkyl, C₁₋₃ alkoxy, C₁₋₃ haloalkyl, C₁₋₃ alkylamino, and di-C₁₋₃ alkylamino.

In some embodiments, R^{a} and R^{b} are each independently selected from the group consisting of halogen, —OH, —NH₂, —CN, and C₁₋₃ alkyl.

In some embodiments, R^{a} and R^{b} are each independently selected from the group consisting of halogen, —OH, —NH₂, and —CN.

In some embodiments, the structural moiety -Cy¹-LNK- is selected from the group consisting of a bond, —CH₂—,

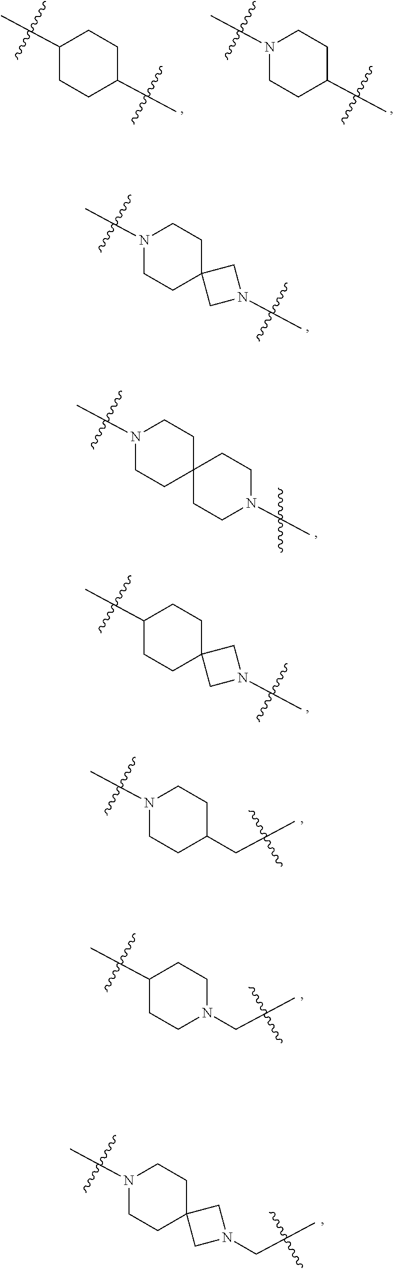

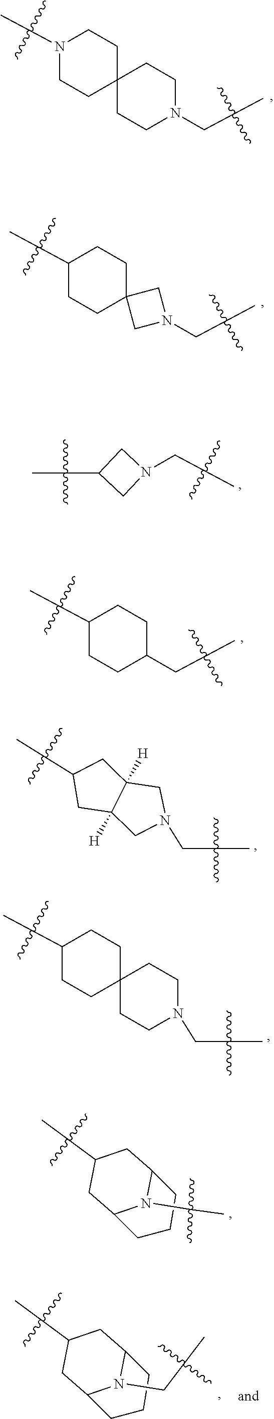

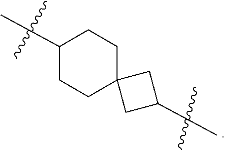 [SMILES: *C1CCC2(CC1)CC(*)C2]

In some specific embodiments, the structural moiety -Cy¹-LNK- is selected from the group consisting of

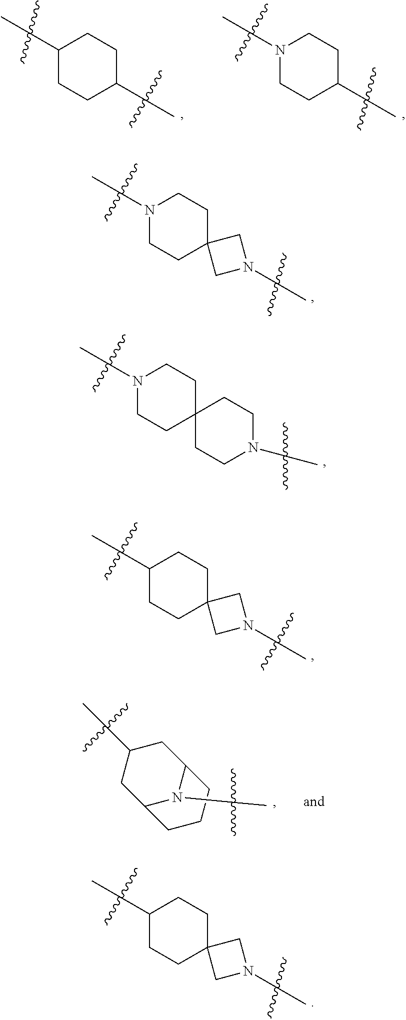

In some specific embodiments, the structural moiety -Cy¹-LNK- is selected from the group consisting of

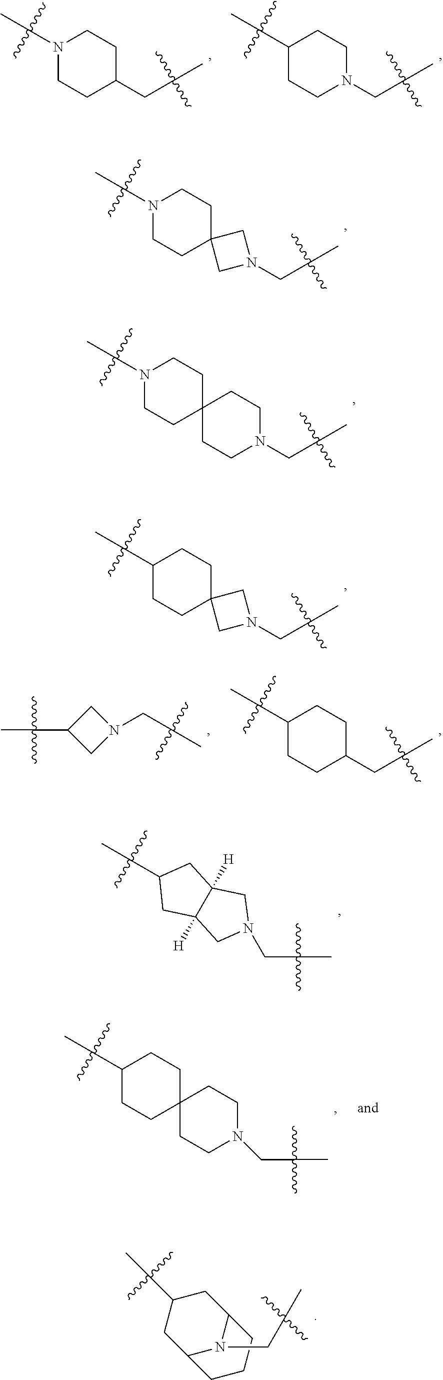

In some specific embodiments, the structural moiety-Cy¹-LNK- is selected from the group consisting of

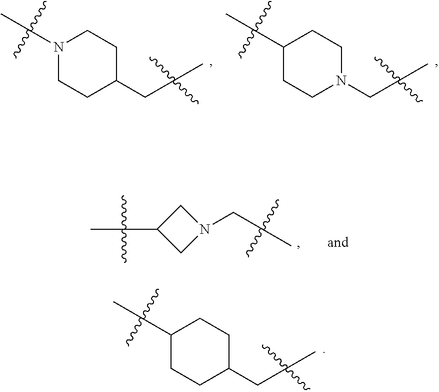 [SMILES: *CC1CCN(*)CC1 ; *CN1CCC(*)CC1 ; *CN1CC(*)C1 ; *CC1CCC(*)CC1]

In some specific embodiments, the structural moiety -Cy¹-LNK- is selected from the group consisting of

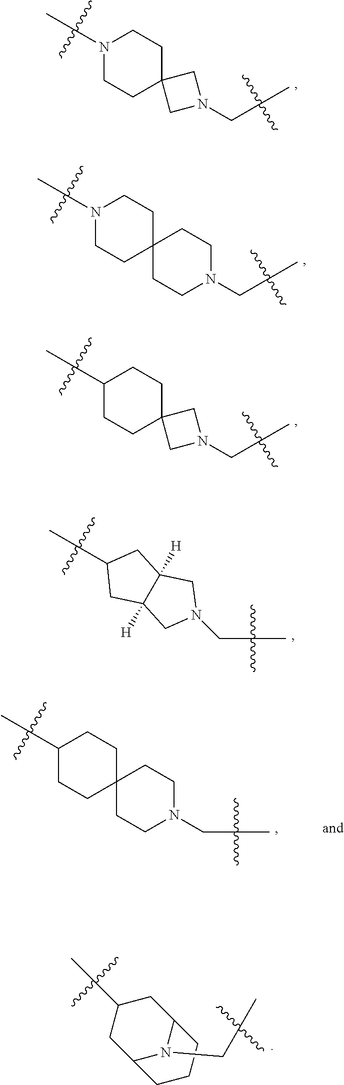 [SMILES: *CN1CC2(CCN(*)CC2)C1 ; *CN1CCC2(CCN(*)CC2)CC1 ; *CN1CC2(CCC(*)CC2)C1 ; *CN1C[C@H]2CC(*)C[C@H]2C1 ; *CN1CCC2(CCC(*)CC2)CC1 ; *CN1C2CCCC1CC(*)C2]

In some other embodiments, the structural moiety -Cy¹-LNK- is selected from the group consisting of

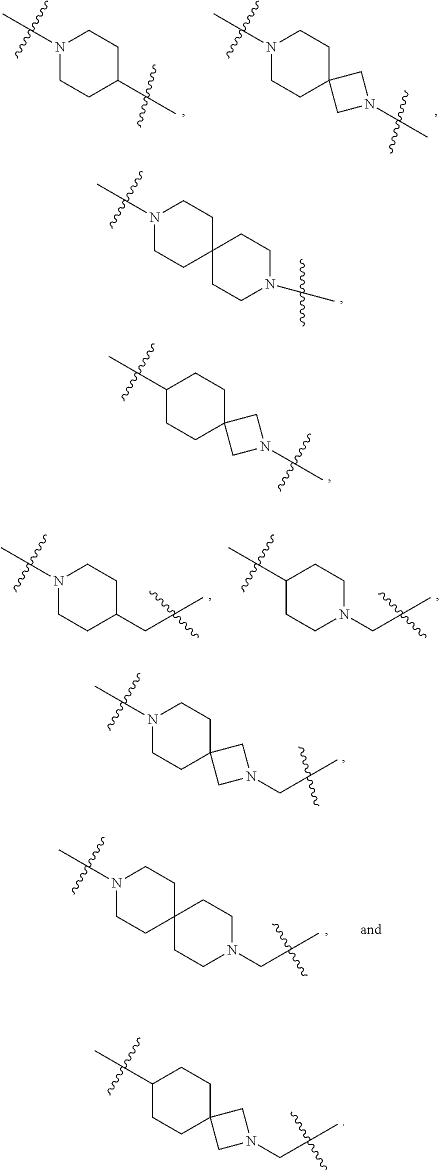

In some embodiments, the structural moiety -LNK-Cy²- is selected from the group consisting of a bond, —CH₂—,

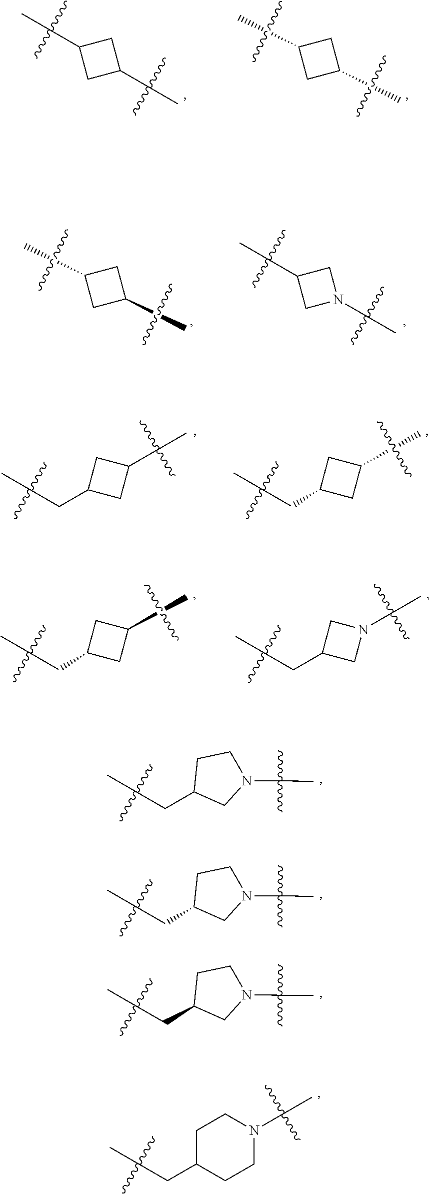

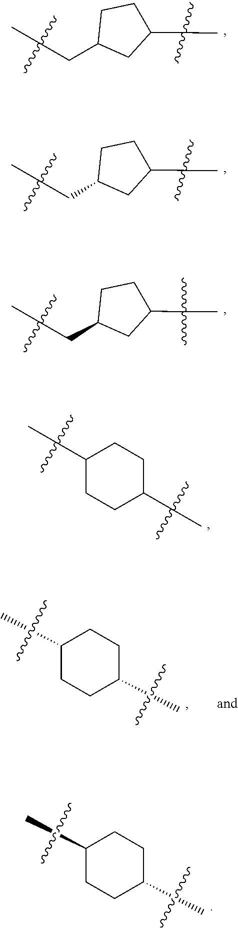

In some other embodiments, the structural moiety -LNK-Cy²- is selected from the group consisting of a bond, —CH₂—,

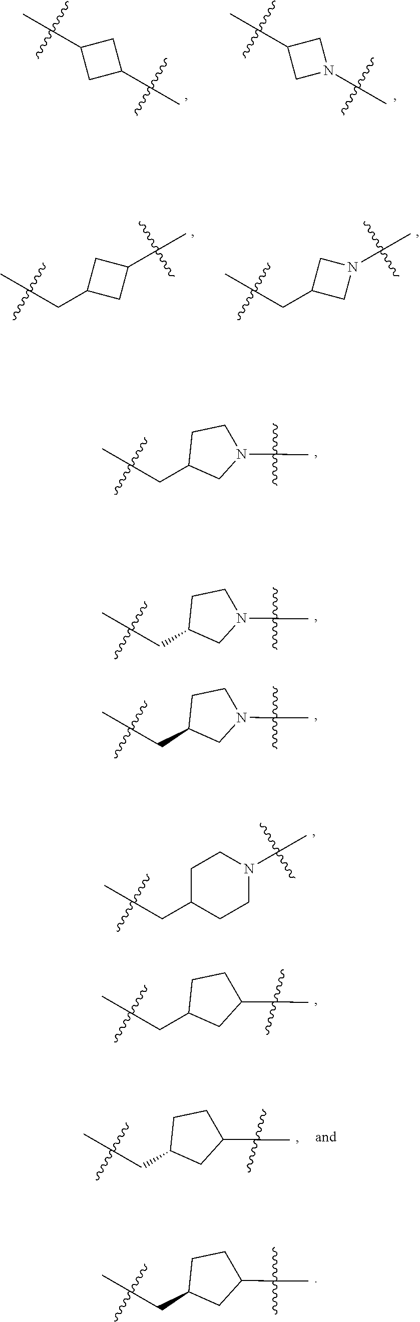

In some embodiments, the structural moiety -Cy¹-Cy²- is selected from the group consisting of,

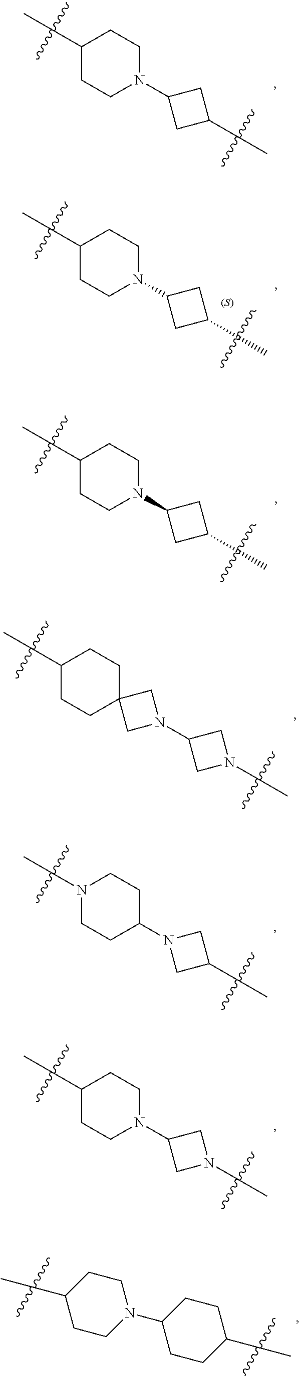

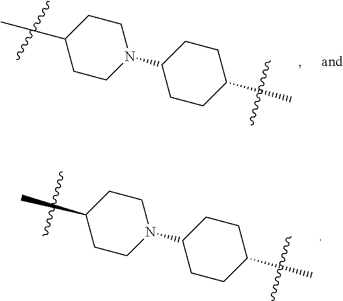

In some other embodiments, the structural moiety -Cy¹-Cy²- is selected from the group consisting of

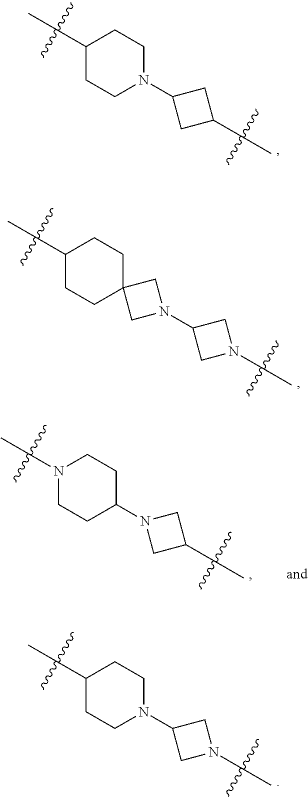 [SMILES: *C1CCN(C2CC(*)C2)CC1 ; *C1CCC2(CC1)CN(C1CN(*)C1)C2 ; *C1CN(C2CCN(*)CC2)C1 ; *C1CCN(C2CN(*)C2)CC1]

In some embodiments, the structural moiety -Cy¹-LNK-Cy²- is selected from the group consisting of a bond, —CH₂—

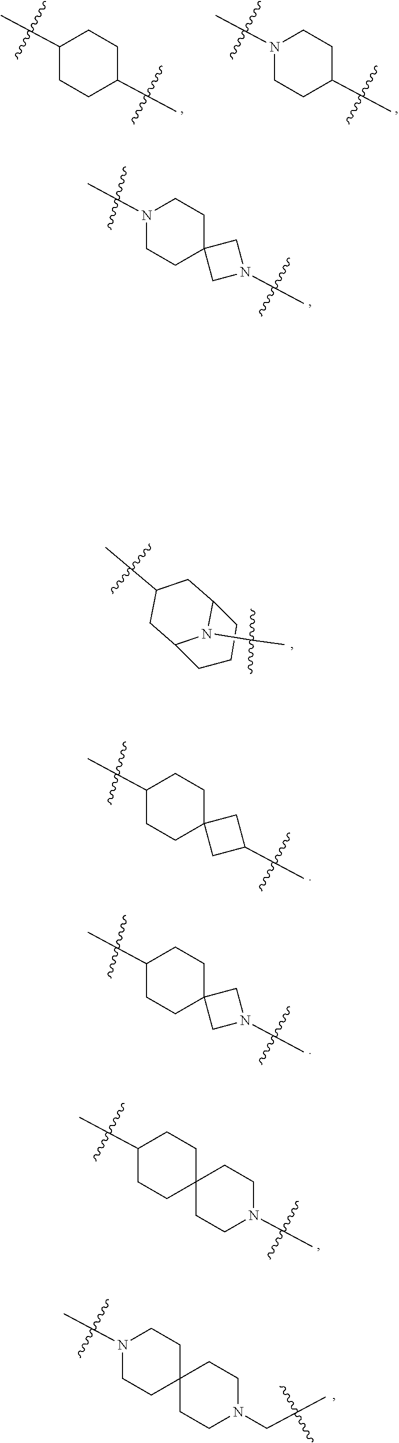

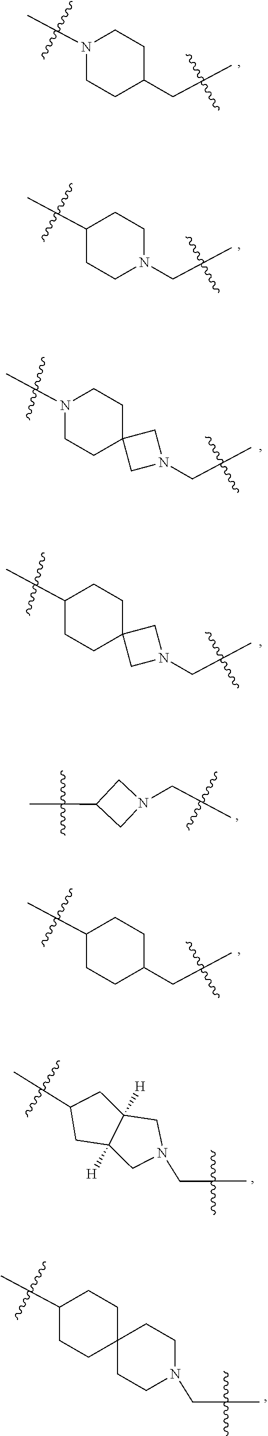

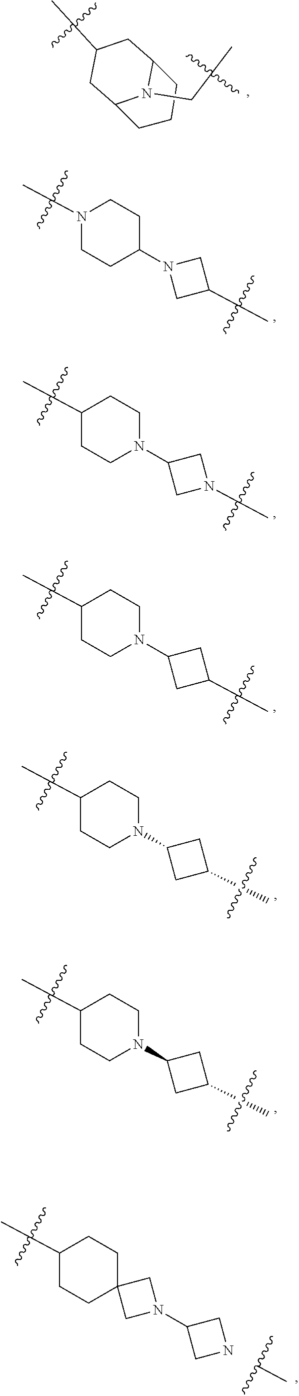

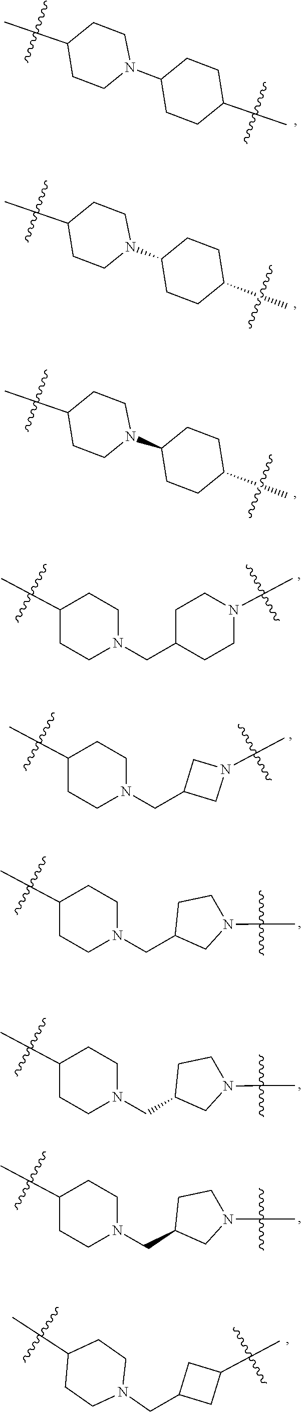

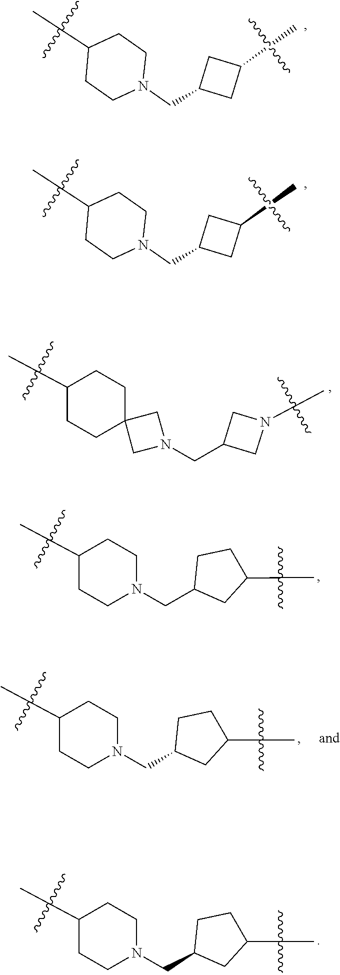

In some specific embodiments, the structural moiety -Cy¹-LNK-Cy²- is selected from the group consisting of

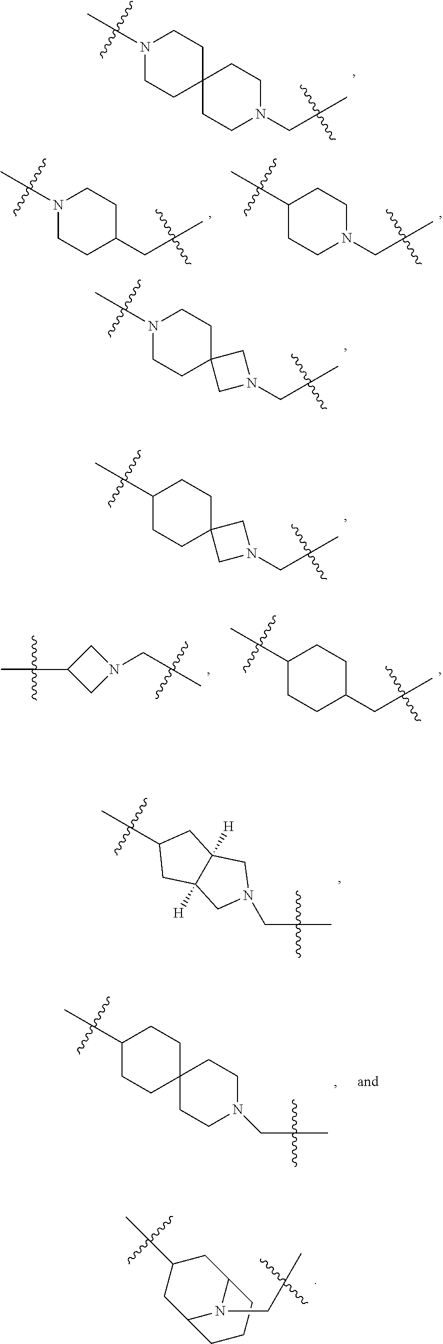

In some specific embodiments, the structural moiety -Cy¹-LNK Cy²- is selected from the group consisting of

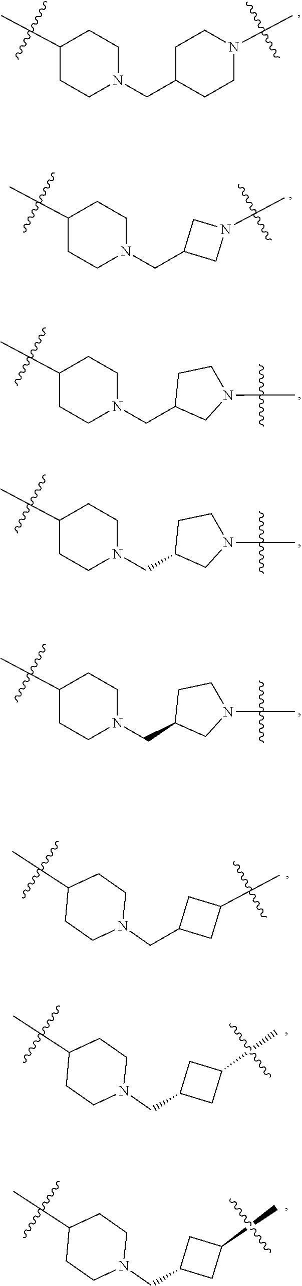

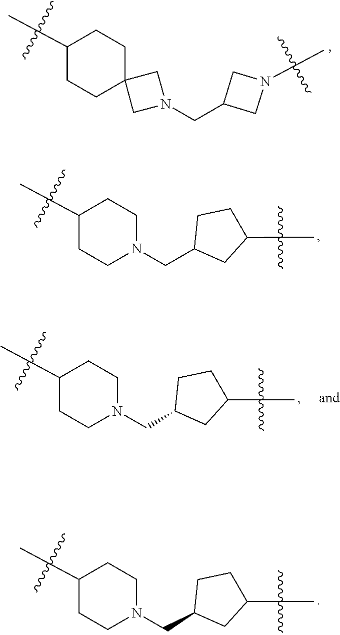 [SMILES: *C1CCC2(CC1)CN(CC1CN(*)C1)C2 ; *C1CCN(CC2CCC(*)C2)CC1 ; *C1CCN(C[C@@H]2CCC(*)C2)CC1 ; *C1CCN(C[C@H]2CCC(*)C2)CC1]

In some embodiments, the structural moiety

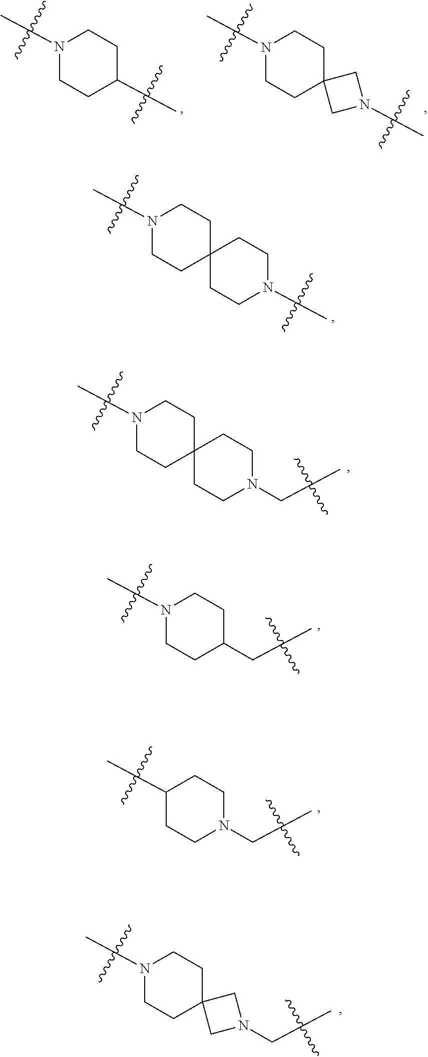

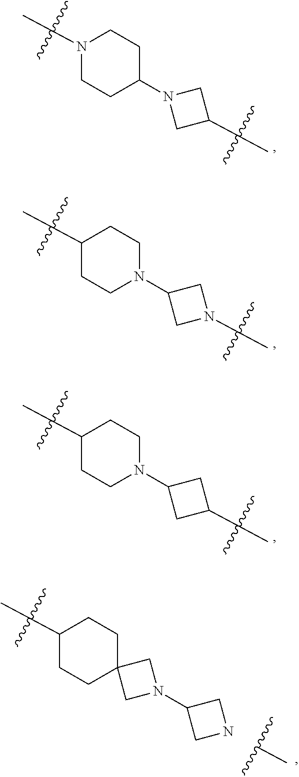

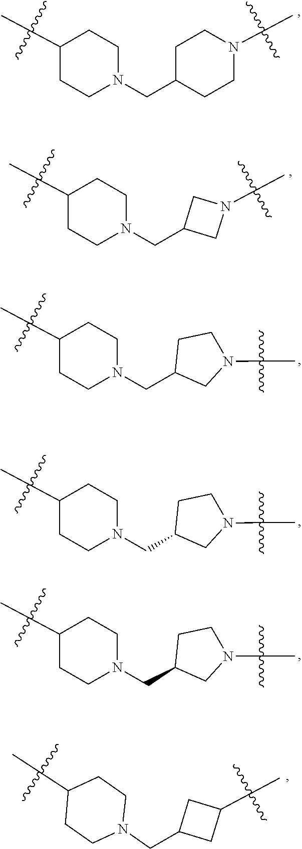 [SMILES: *C1CCN(CC2CCN(*)CC2)CC1 ; *C1CCN(CC2CN(*)C2)CC1 ; *C1CCN(CC2CCN(*)C2)CC1 ; *C1CCN(C[C@@H]2CCN(*)C2)CC1 ; *C1CCN(C[C@H]2CCN(*)C2)CC1 ; *C1CCN(CC2CC(*)C2)CC1]

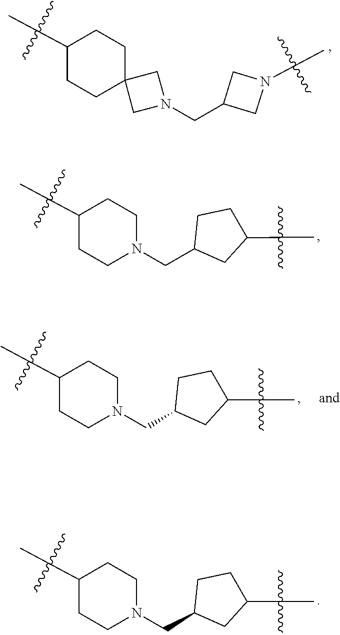 [SMILES: *C1CCC2(CC1)CN(CC1CN(*)C1)C2 ; *C1CCN(CC2CCC(*)C2)CC1 ; *C1CCN(C[C@@H]2CCC(*)C2)CC1 ; *C1CCN(C[C@H]2CCC(*)C2)CC1]

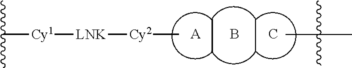

is selected from the group consisting of

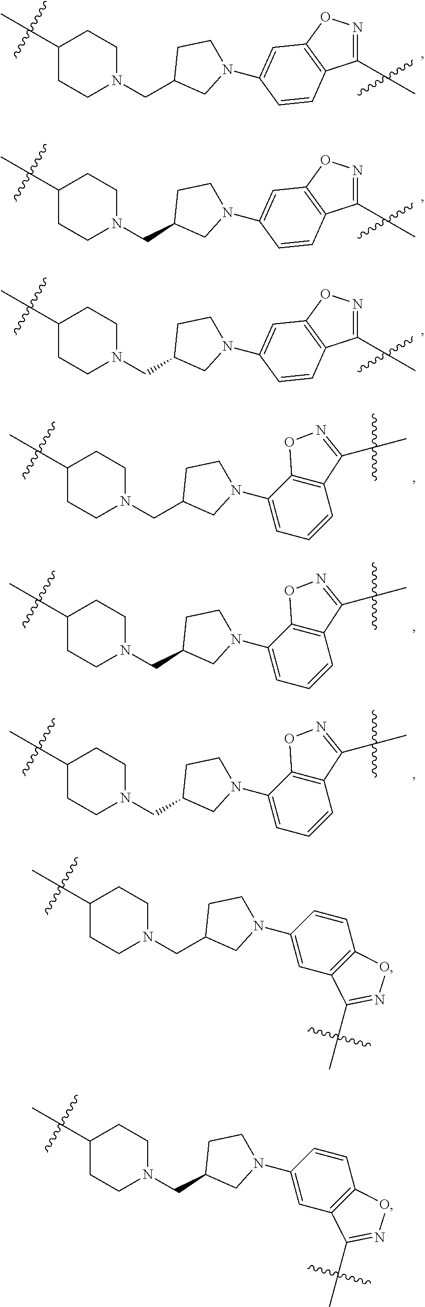

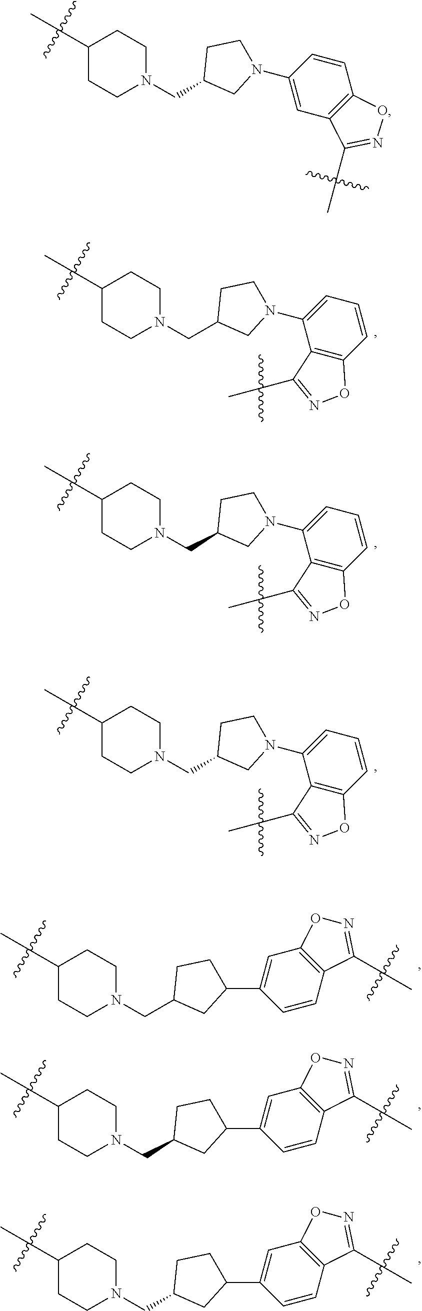

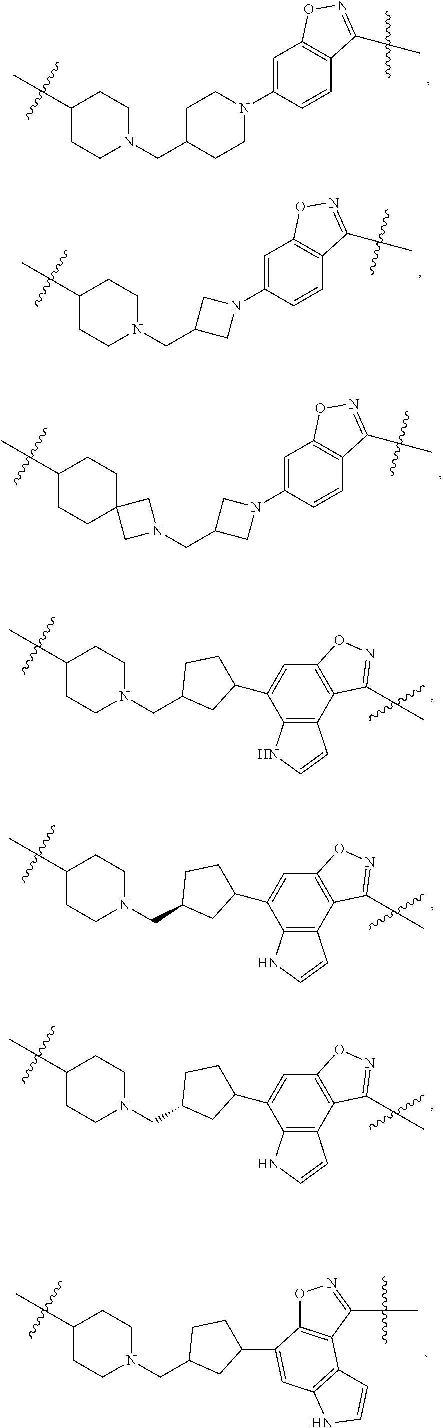

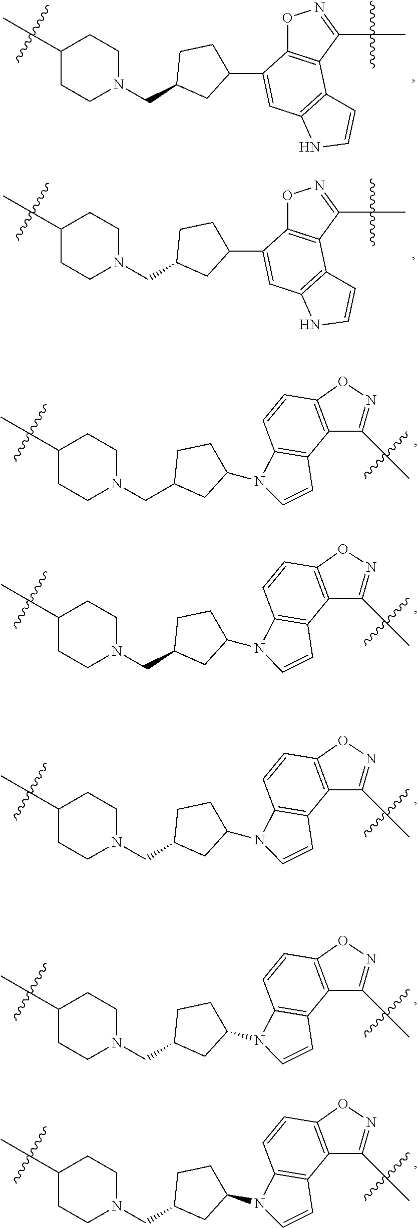

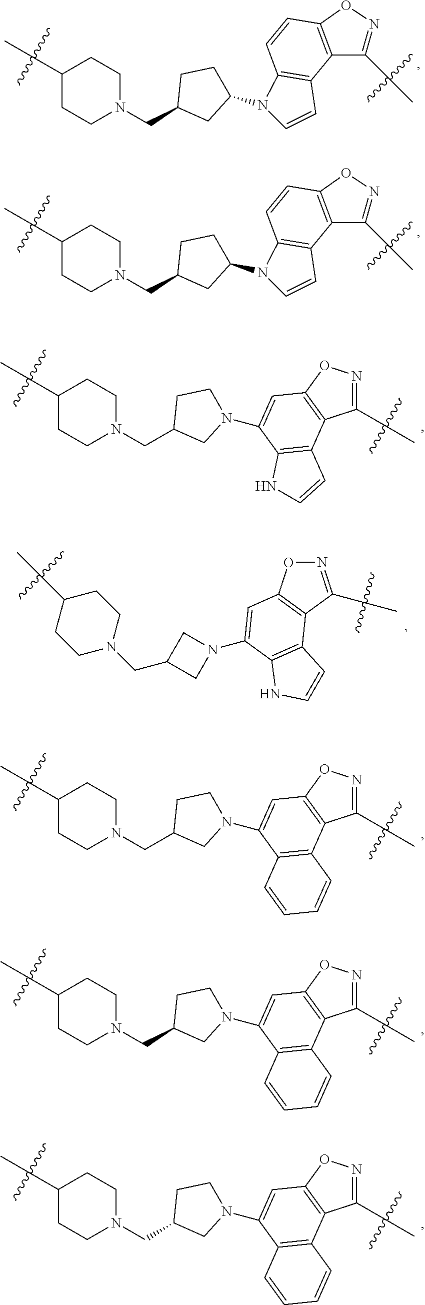

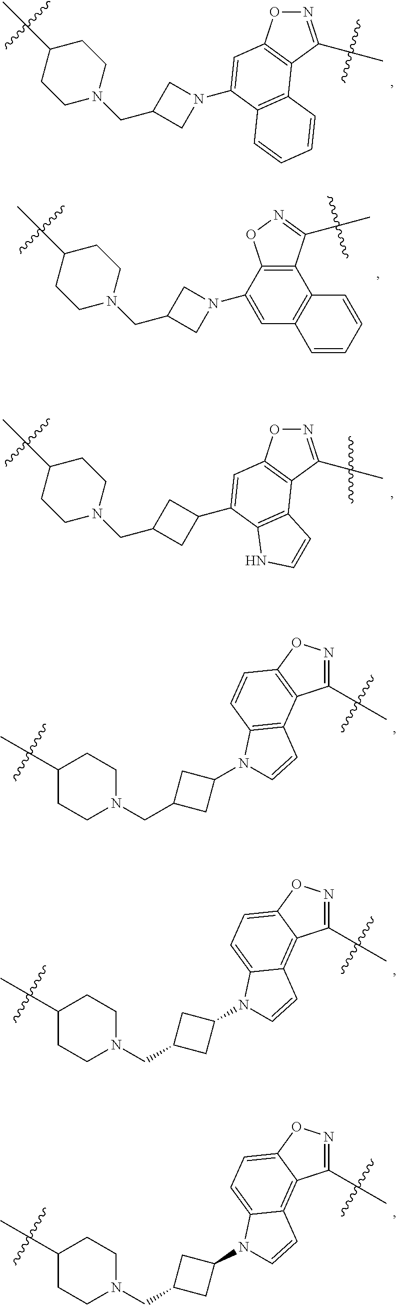 [SMILES: *c1noc2cc(N3CC(CN4CCC(*)CC4)C3)c3ccccc3c12 ; *c1noc2c(N3CC(CN4CCC(*)CC4)C3)cc3ccccc3c12 ; *c1noc2cc(C3CC(CN4CCC(*)CC4)C3)c3[nH]ccc3c12 ; *c1noc2ccc3c(ccn3C3CC(CN4CCC(*)CC4)C3)c12 ; *c1noc2ccc3c(ccn3[C@H]3C[C@@H](CN4CCC(*)CC4)C3)c12 ; *c1noc2ccc3c(ccn3[C@H]3C[C@H](CN4CCC(*)CC4)C3)c12]

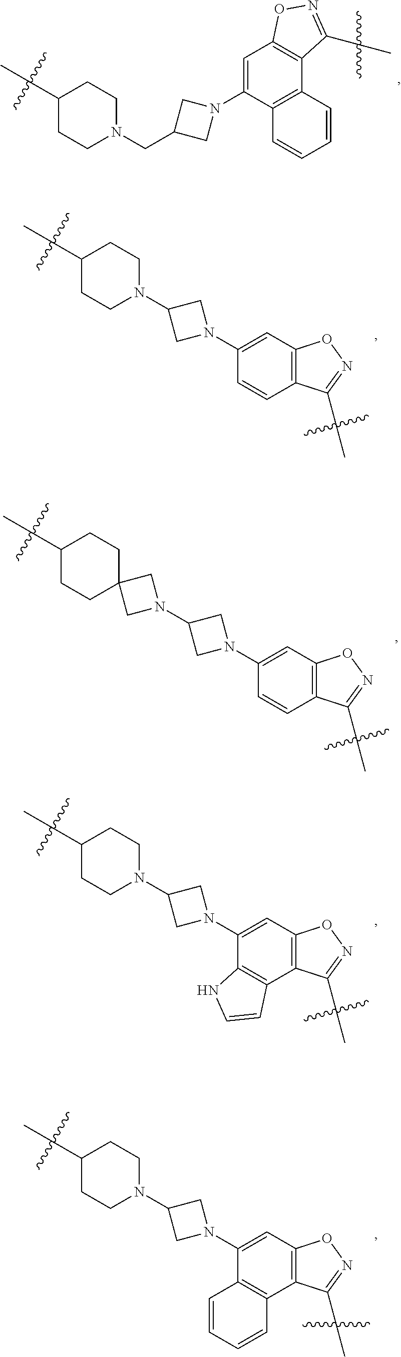 [SMILES: *c1noc2cc(N3CC(CN4CCC(*)CC4)C3)c3ccccc3c12 ; *c1noc2cc(N3CC(N4CCC(*)CC4)C3)ccc12 ; *c1noc2cc(N3CC(N4CC5(CCC(*)CC5)C4)C3)ccc12 ; *c1noc2cc(N3CC(N4CCC(*)CC4)C3)c3[nH]ccc3c12 ; *c1noc2cc(N3CC(N4CCC(*)CC4)C3)c3ccccc3c12]

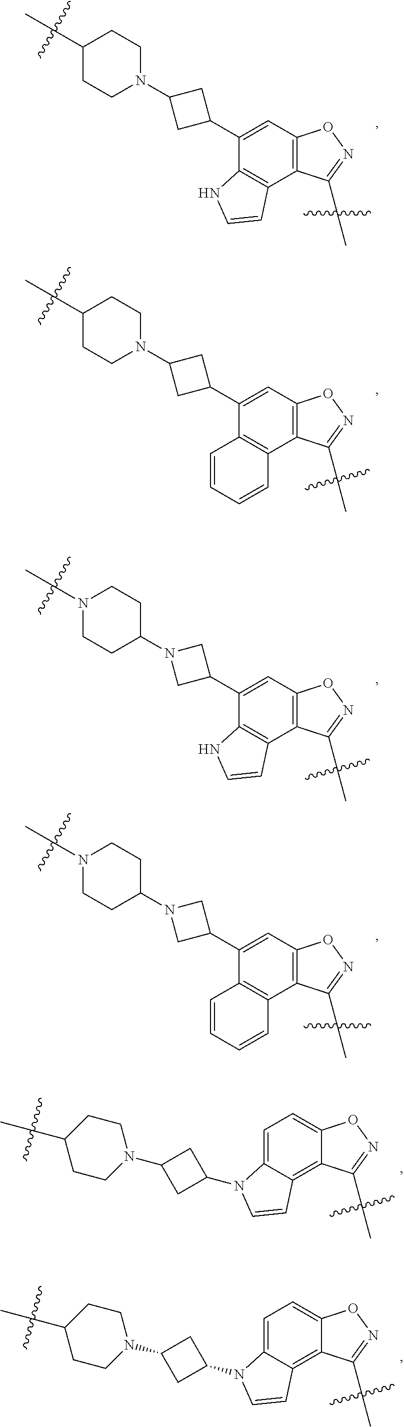 [SMILES: *c1noc2cc(C3CC(N4CCC(*)CC4)C3)c3[nH]ccc3c12 ; *c1noc2cc(C3CC(N4CCC(*)CC4)C3)c3ccccc3c12 ; *c1noc2cc(C3CN(C4CCN(*)CC4)C3)c3[nH]ccc3c12 ; *c1noc2cc(C3CN(C4CCN(*)CC4)C3)c3ccccc3c12 ; *c1noc2ccc3c(ccn3C3CC(N4CCC(*)CC4)C3)c12 ; *c1noc2ccc3c(ccn3[C@H]3C[C@@H](N4CCC(*)CC4)C3)c12]

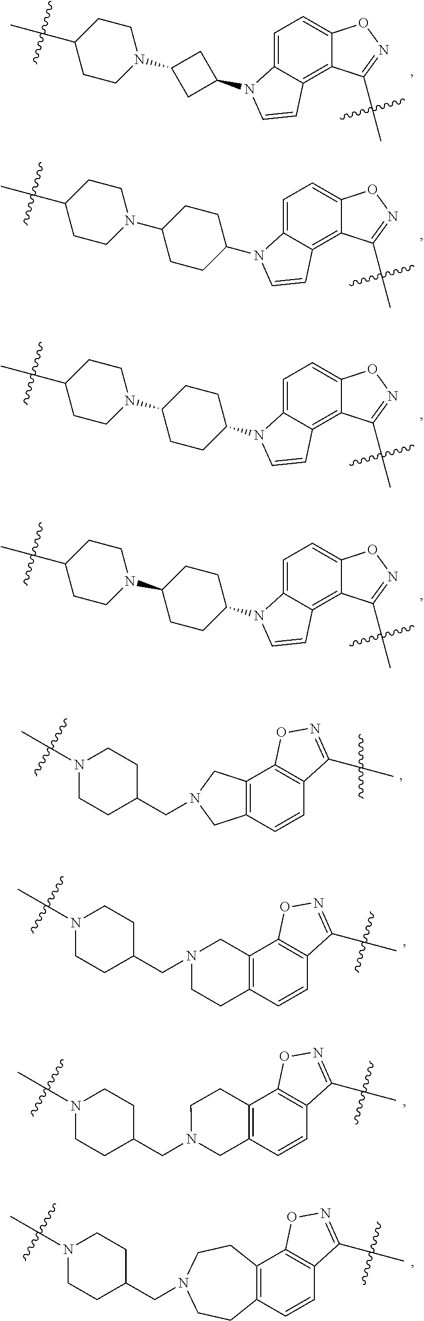

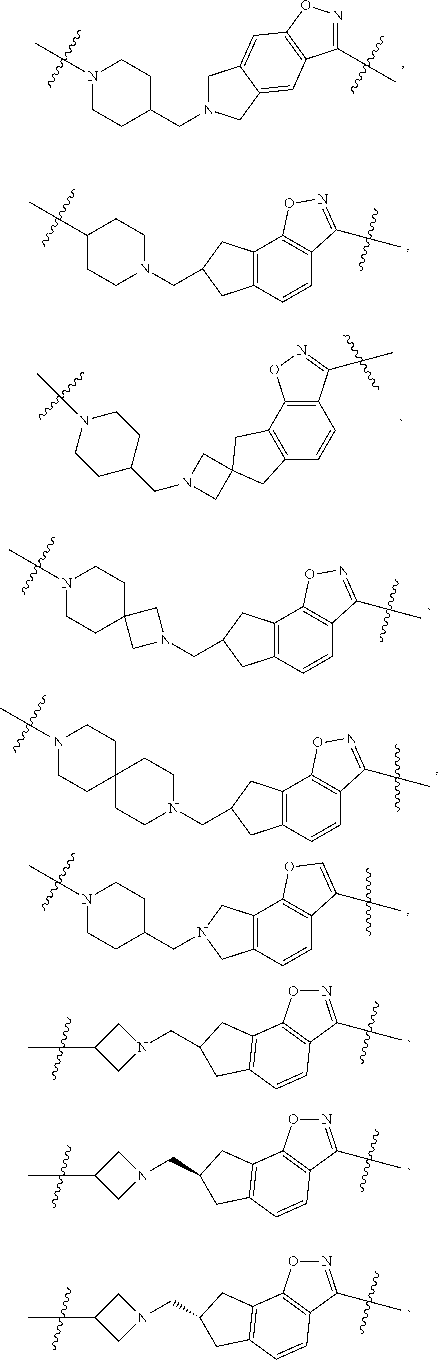

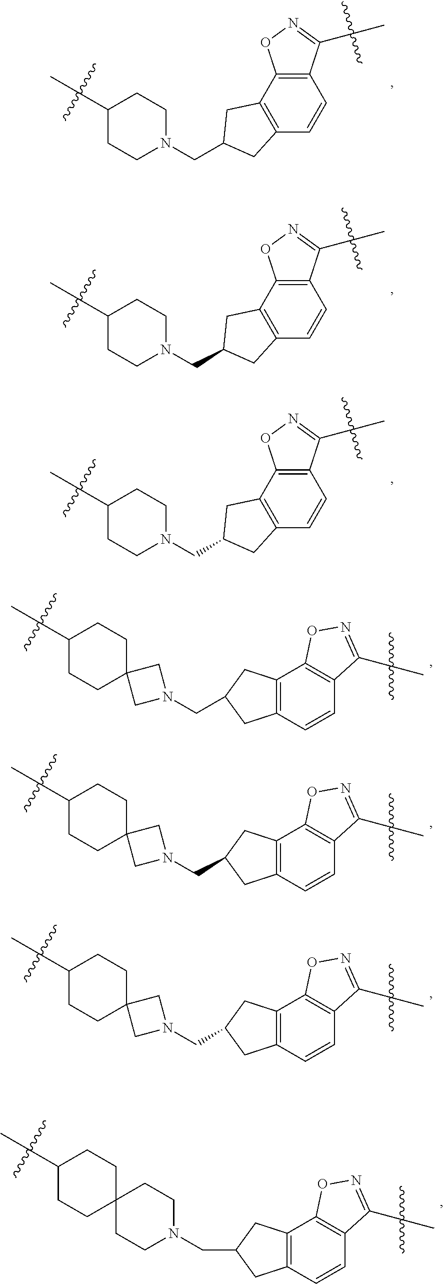

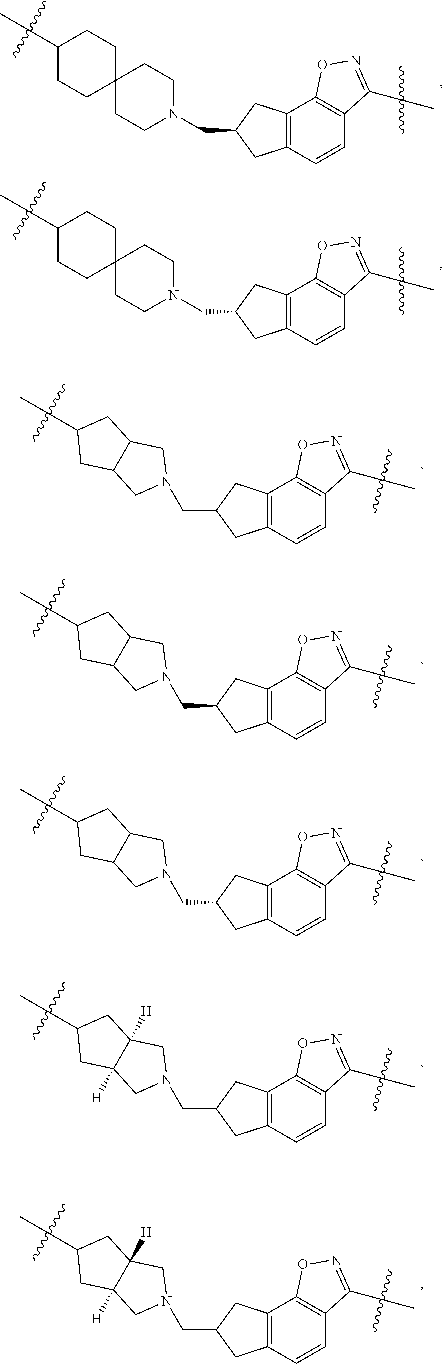

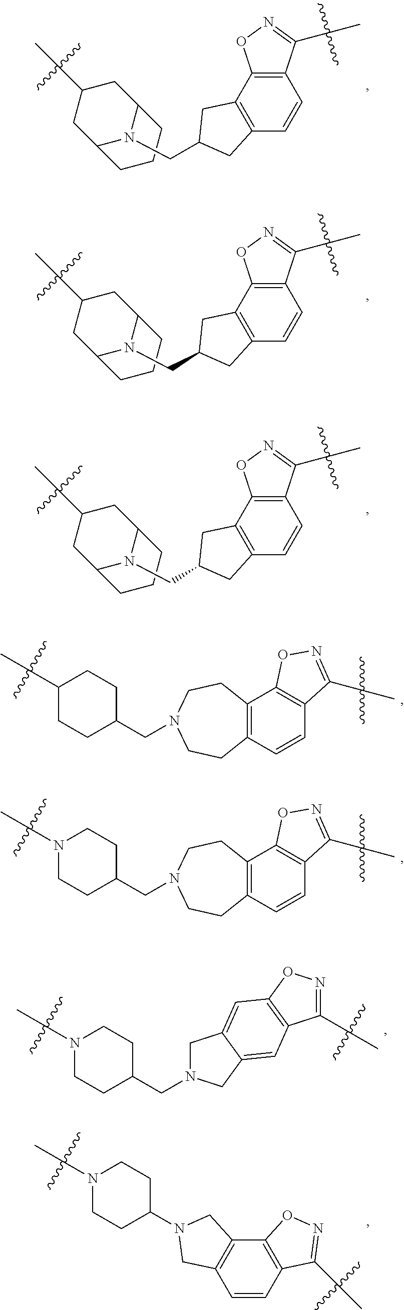

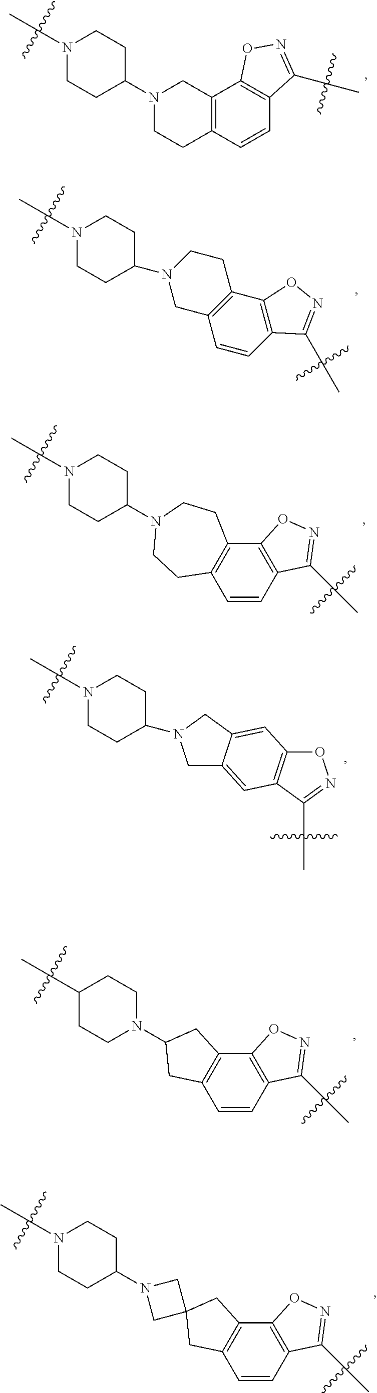 [SMILES: *c1noc2c3c(ccc12)CCN(C1CCN(*)CC1)C3 ; *c1noc2c3c(ccc12)CN(C1CCN(*)CC1)CC3 ; *c1noc2c3c(ccc12)CCN(C1CCN(*)CC1)CC3 ; *c1noc2cc3c(cc12)CN(C1CCN(*)CC1)C3 ; *c1noc2c3c(ccc12)CC(N1CCC(*)CC1)C3 ; *c1noc2c3c(ccc12)CC1(C3)CN(C2CCN(*)CC2)C1]

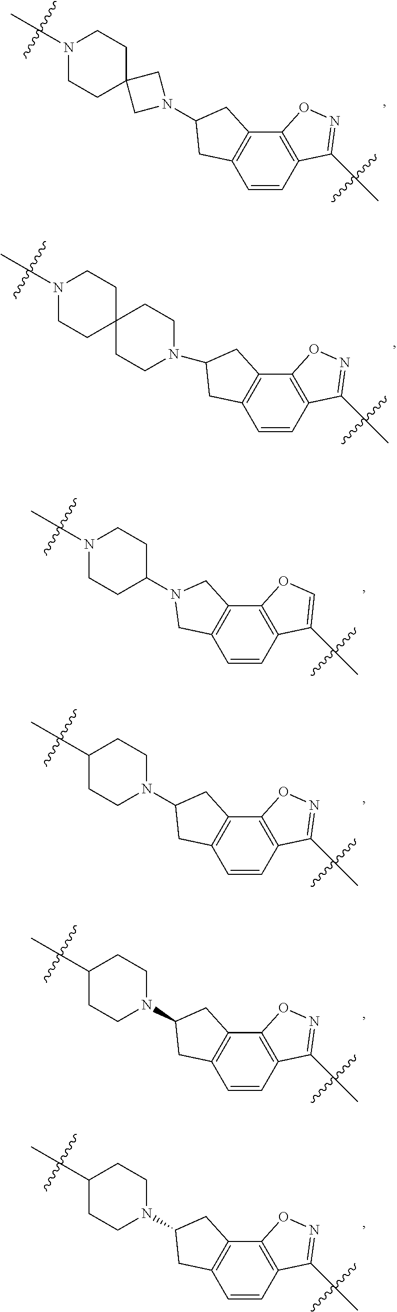 [SMILES: *c1noc2c3c(ccc12)CC(N1CC2(CCN(*)CC2)C1)C3 ; *c1noc2c3c(ccc12)CC(N1CCC2(CCN(*)CC2)CC1)C3 ; *c1coc2c3c(ccc12)CN(C1CCN(*)CC1)C3 ; *c1noc2c3c(ccc12)CC(N1CCC(*)CC1)C3 ; *c1noc2c3c(ccc12)C[C@@H](N1CCC(*)CC1)C3 ; *c1noc2c3c(ccc12)C[C@H](N1CCC(*)CC1)C3]

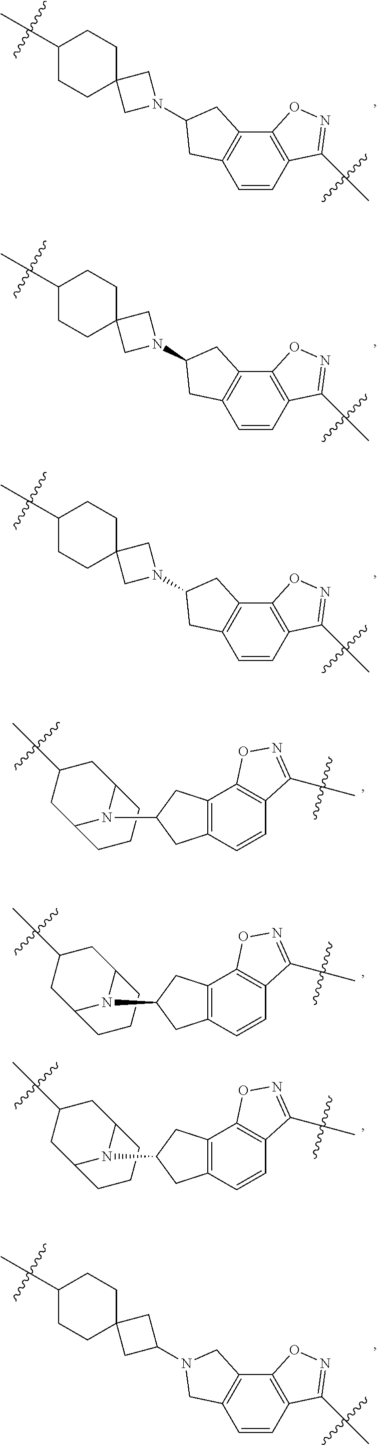

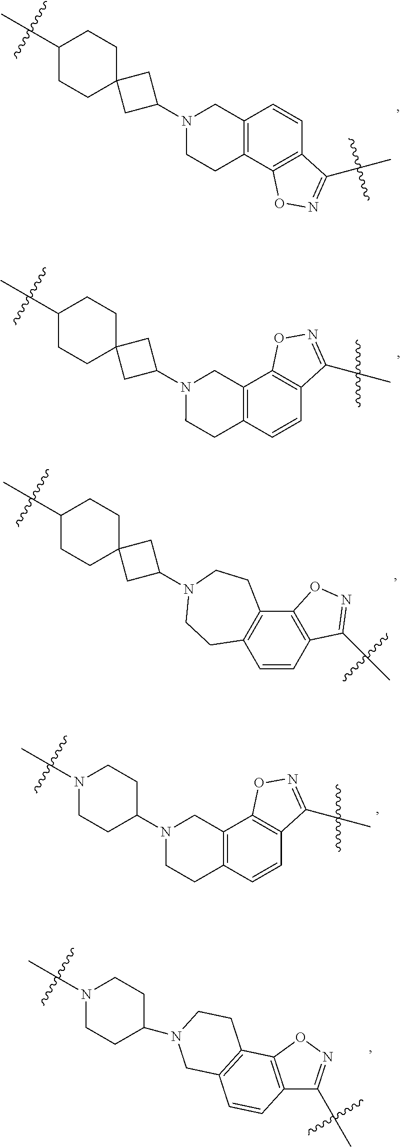 [SMILES: *c1noc2c3c(ccc12)CN(C1CC2(CCC(*)CC2)C1)CC3 ; *c1noc2c3c(ccc12)CCN(C1CC2(CCC(*)CC2)C1)C3 ; *c1noc2c3c(ccc12)CCN(C1CC2(CCC(*)CC2)C1)CC3 ; *c1noc2c3c(ccc12)CCN(C1CCN(*)CC1)C3 ; *c1noc2c3c(ccc12)CN(C1CCN(*)CC1)CC3]

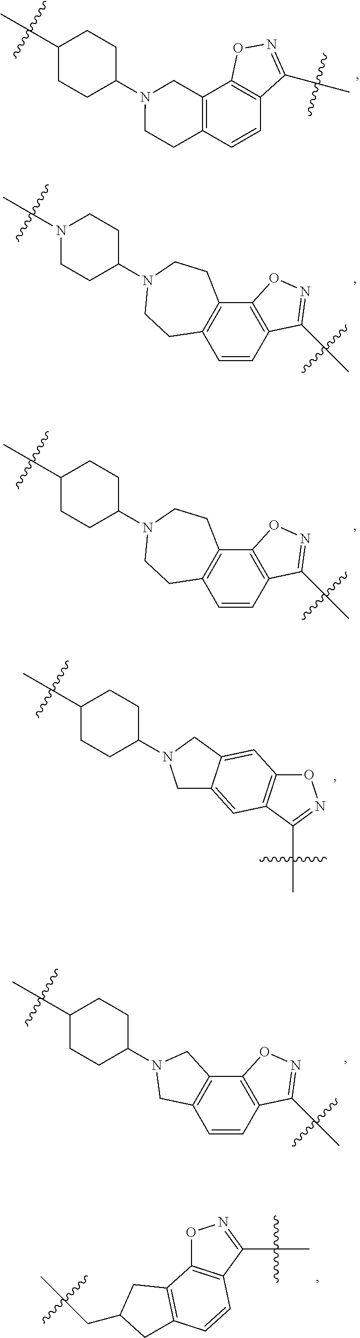 [SMILES: *c1noc2c3c(ccc12)CCN(C1CCC(*)CC1)C3 ; *c1noc2c3c(ccc12)CCN(C1CCN(*)CC1)CC3 ; *c1noc2c3c(ccc12)CCN(C1CCC(*)CC1)CC3 ; *c1noc2cc3c(cc12)CN(C1CCC(*)CC1)C3 ; *c1noc2c3c(ccc12)CN(C1CCC(*)CC1)C3 ; *CC1Cc2ccc3c(*)noc3c2C1]

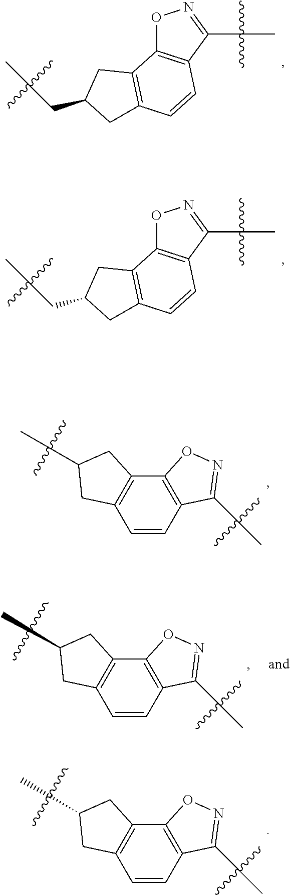 [SMILES: *C[C@@H]1Cc2ccc3c(*)noc3c2C1 ; *C[C@H]1Cc2ccc3c(*)noc3c2C1 ; *c1noc2c3c(ccc12)CC(*)C3 ; *c1noc2c3c(ccc12)C[C@@H](C)C3 ; *c1noc2c3c(ccc12)C[C@H](C)C3]

In some other embodiments the structural moiety

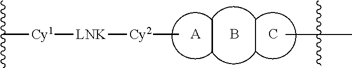

is selected from the group consisting of

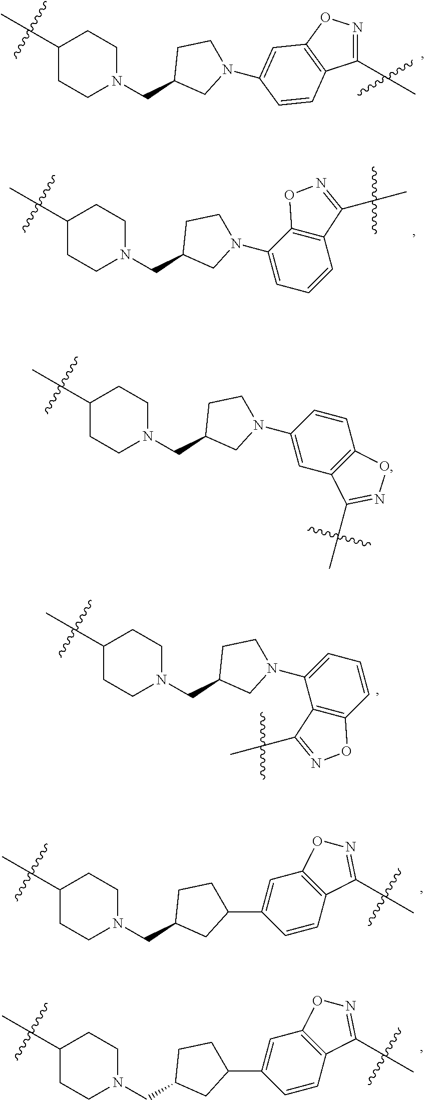 [SMILES: *c1noc2cc(N3CC[C@H](CN4CCC(*)CC4)C3)ccc12 ; *c1noc2c(N3CC[C@H](CN4CCC(*)CC4)C3)cccc12 ; *c1noc2ccc(N3CC[C@H](CN4CCC(*)CC4)C3)cc12 ; *c1noc2cccc(N3CC[C@H](CN4CCC(*)CC4)C3)c12 ; *c1noc2cc(C3CC[C@H](CN4CCC(*)CC4)C3)ccc12 ; *c1noc2cc(C3CC[C@@H](CN4CCC(*)CC4)C3)ccc12]

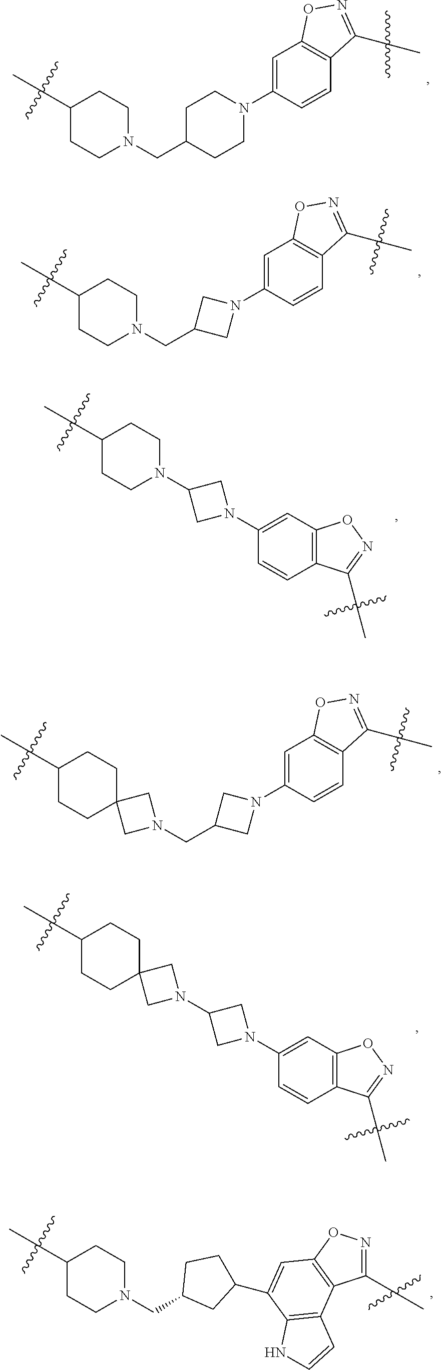 [SMILES: *c1noc2cc(N3CCC(CN4CCC(*)CC4)CC3)ccc12 ; *c1noc2cc(N3CC(CN4CCC(*)CC4)C3)ccc12 ; *c1noc2cc(N3CC(N4CCC(*)CC4)C3)ccc12 ; *c1noc2cc(N3CC(CN4CC5(CCC(*)CC5)C4)C3)ccc12 ; *c1noc2cc(N3CC(N4CC5(CCC(*)CC5)C4)C3)ccc12 ; *c1noc2cc(C3CC[C@@H](CN4CCC(*)CC4)C3)c3[nH]ccc3c12]

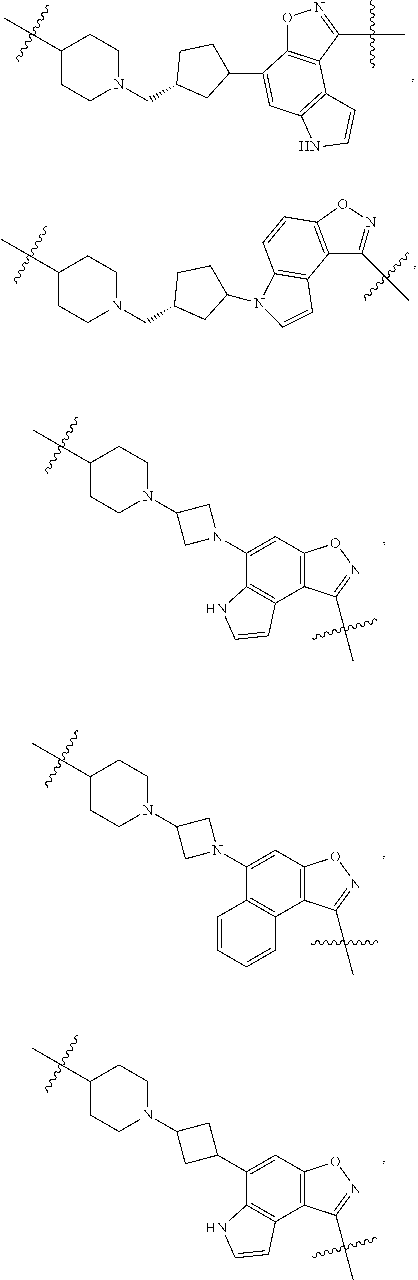 [SMILES: *c1noc2c(C3CC[C@@H](CN4CCC(*)CC4)C3)cc3[nH]ccc3c12 ; *c1noc2ccc3c(ccn3C3CC[C@@H](CN4CCC(*)CC4)C3)c12 ; *c1noc2cc(N3CC(N4CCC(*)CC4)C3)c3[nH]ccc3c12 ; *c1noc2cc(N3CC(N4CCC(*)CC4)C3)c3ccccc3c12 ; *c1noc2cc(C3CC(N4CCC(*)CC4)C3)c3[nH]ccc3c12]

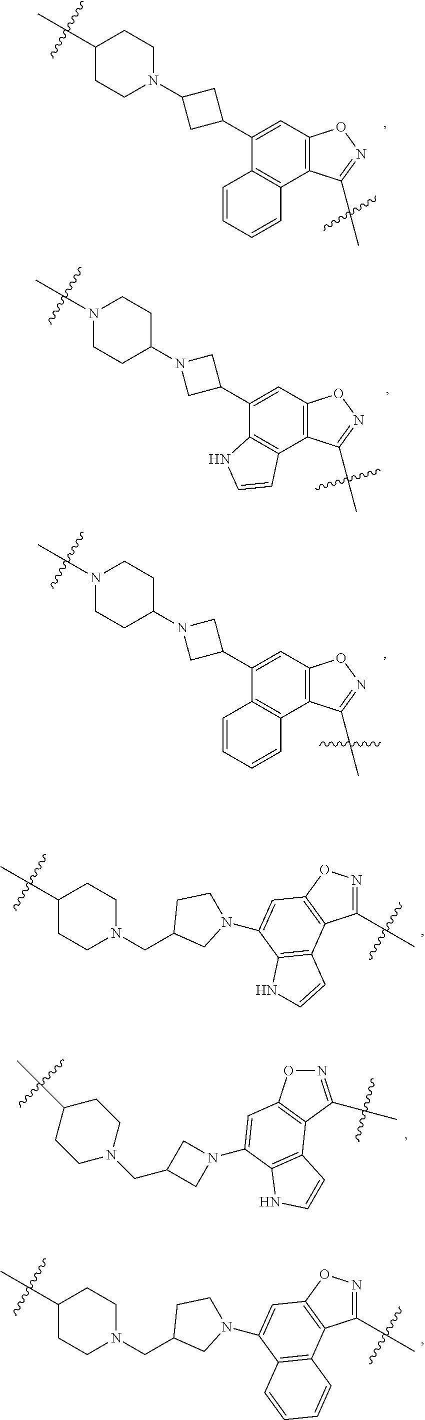 [SMILES: *c1noc2cc(C3CC(N4CCC(*)CC4)C3)c3ccccc3c12 ; *c1noc2cc(C3CN(C4CCN(*)CC4)C3)c3[nH]ccc3c12 ; *c1noc2cc(C3CN(C4CCN(*)CC4)C3)c3ccccc3c12 ; *c1noc2cc(N3CCC(CN4CCC(*)CC4)C3)c3[nH]ccc3c12 ; *c1noc2cc(N3CC(CN4CCC(*)CC4)C3)c3[nH]ccc3c12 ; *c1noc2cc(N3CCC(CN4CCC(*)CC4)C3)c3ccccc3c12]

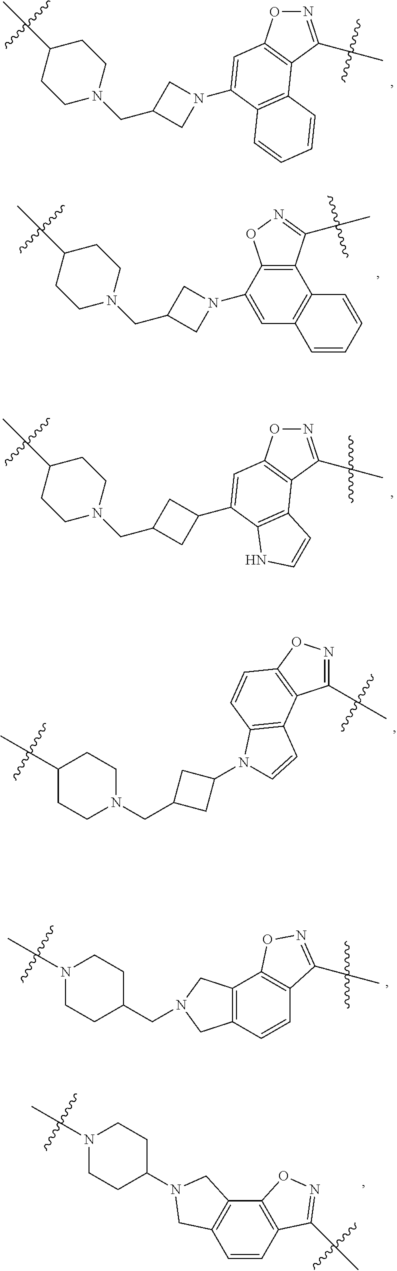 [SMILES: *c1noc2cc(N3CC(CN4CCC(*)CC4)C3)c3ccccc3c12 ; *c1noc2c(N3CC(CN4CCC(*)CC4)C3)cc3ccccc3c12 ; *c1noc2cc(C3CC(CN4CCC(*)CC4)C3)c3[nH]ccc3c12 ; *c1noc2ccc3c(ccn3C3CC(CN4CCC(*)CC4)C3)c12 ; *c1noc2c3c(ccc12)CN(CC1CCN(*)CC1)C3 ; *c1noc2c3c(ccc12)CN(C1CCN(*)CC1)C3]

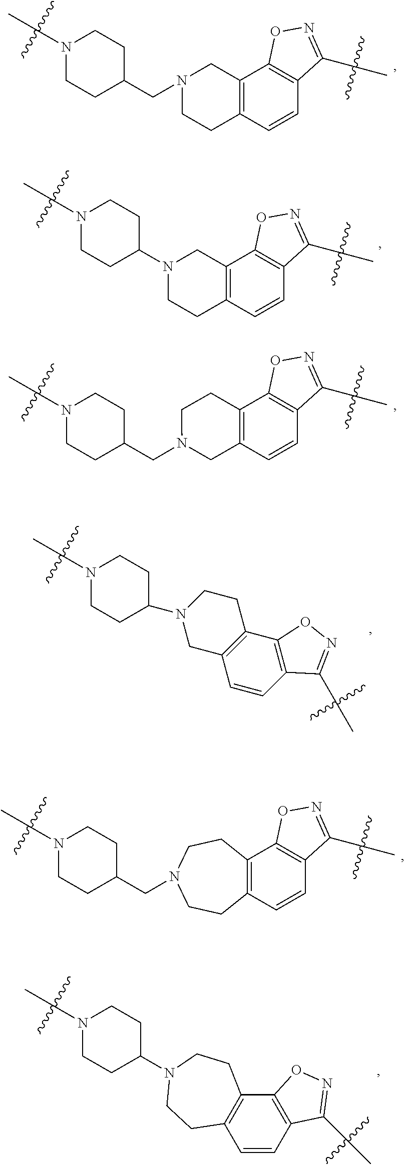 [SMILES: *c1noc2c3c(ccc12)CCN(CC1CCN(*)CC1)C3 ; *c1noc2c3c(ccc12)CCN(C1CCN(*)CC1)C3 ; *c1noc2c3c(ccc12)CN(CC1CCN(*)CC1)CC3 ; *c1noc2c3c(ccc12)CN(C1CCN(*)CC1)CC3 ; *c1noc2c3c(ccc12)CCN(CC1CCN(*)CC1)CC3 ; *c1noc2c3c(ccc12)CCN(C1CCN(*)CC1)CC3]

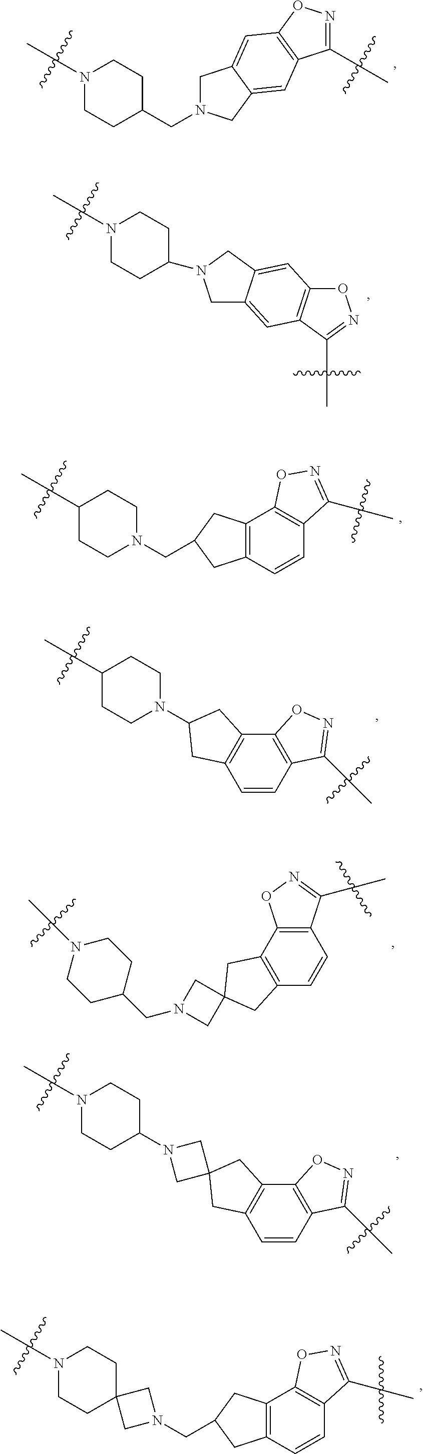

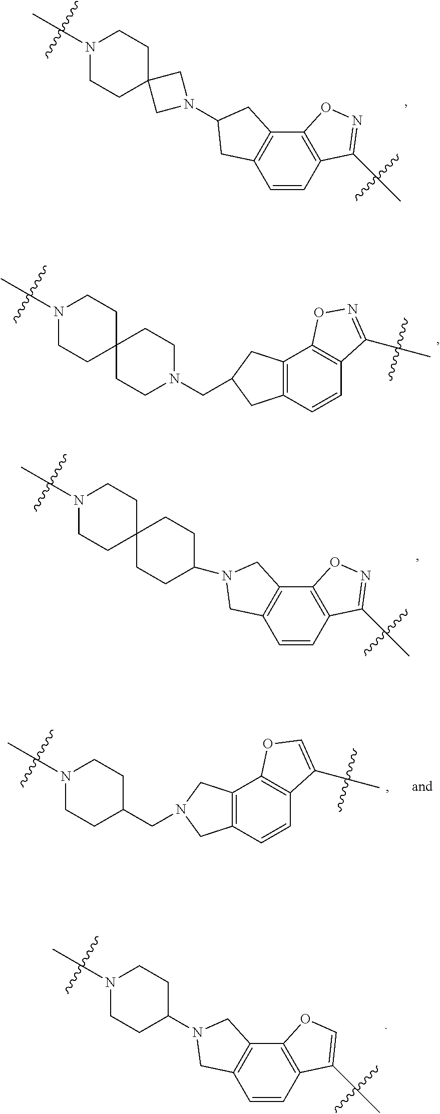 [SMILES: *c1noc2c3c(ccc12)CC(N1CC2(CCN(*)CC2)C1)C3 ; *c1noc2c3c(ccc12)CC(CN1CCC2(CCN(*)CC2)CC1)C3 ; *c1noc2c3c(ccc12)CN(C1CCC2(CC1)CCN(*)CC2)C3 ; *c1coc2c3c(ccc12)CN(CC1CCN(*)CC1)C3 ; *c1coc2c3c(ccc12)CN(C1CCN(*)CC1)C3]

In some embodiments, T is selected from CH. In some embodiments, T is selected from N.

In some embodiments, R is selected from the group consisting of hydrogen and 5- to 6-membered heterocycloalkyl, wherein the heterocycloalkyl is optionally substituted with ═O or C₁₋₃ alkyl.

In some embodiments, R is selected from the group consisting of hydrogen and 5-membered heterocycloalkyl, wherein the heterocycloalkyl is optionally substituted with ═O or C₁₋₃ alkyl.

In some embodiments, R is selected from the group consisting of hydrogen and imidazolinyl, wherein the imidazolinyl is optionally substituted with ═O or methyl.

In some embodiments, R is selected from the group consisting of hydrogen and

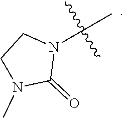 [SMILES: *N1CCN(C)C1=O]

In some embodiments, the present application relates to a compound of formula I, a stereoisomer thereof, or a pharmaceutically acceptable salt thereof, wherein, ring A is absent or selected from the group consisting of C₅₋₈ cycloalkenyl, 5- to 9-membered heterocycloalkenyl (preferably 5- to 8-membered heterocycloalkenyl or 5- to 7-membered heterocycloalkenyl), and phenyl; or, ring A is absent or selected from the group consisting of C₅₋₆ cycloalkenyl, 5- to 9-membered heterocycloalkenyl (preferably 5- to 8-membered heterocycloalkenyl or 5- to 7-membered heterocycloalkenyl), and phenyl; preferably, ring A is absent or selected from the group consisting of C₅₋₆ cycloalkenyl, 5- to 7-membered heterocycloalkenyl containing 1-2 heteroatoms selected from the group consisting of N, O, and S (preferably containing 1-2 N atoms, e.g., 1 N atom), and phenyl; or, preferably, ring A is selected from the group consisting of C₅₋₆ cycloalkenyl and 5- to 7-membered heterocycloalkenyl containing 1-2 heteroatoms selected from the group consisting of N, O, and S (preferably containing 1-2 N atoms, e.g., 1 N atom);
ring B is selected from phenyl;
ring C is selected from the group consisting of isoxazolyl and furanyl;
each R¹ is independently selected from the group consisting of fluorine, chlorine, and bromine; n is selected from the group consisting of 0, 1, and 2, preferably 0 and 1;
preferably,

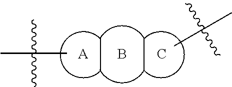

is selected from the group consisting of

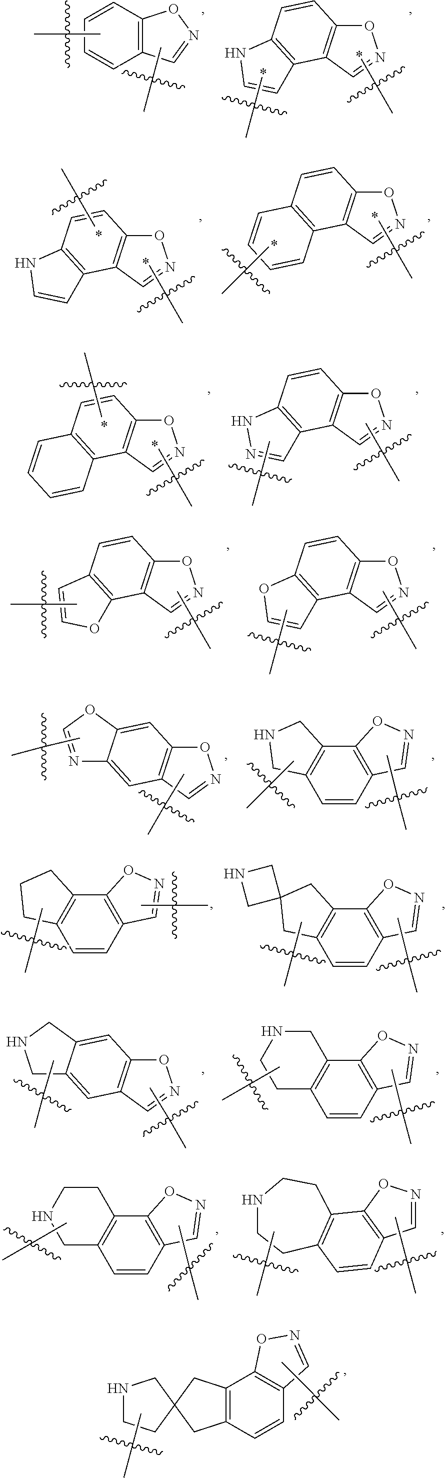

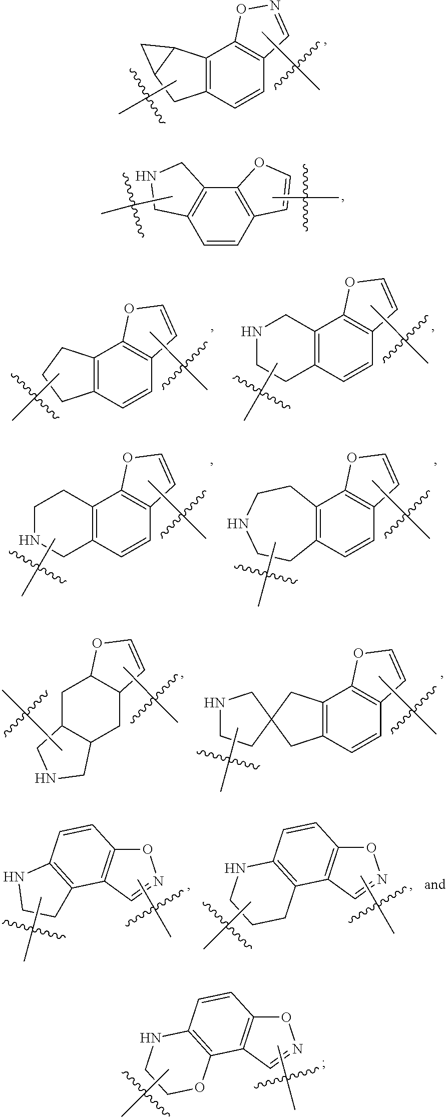

PTM is

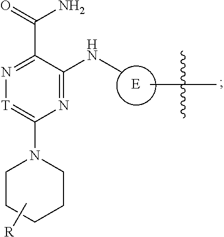

preferably, PTM is

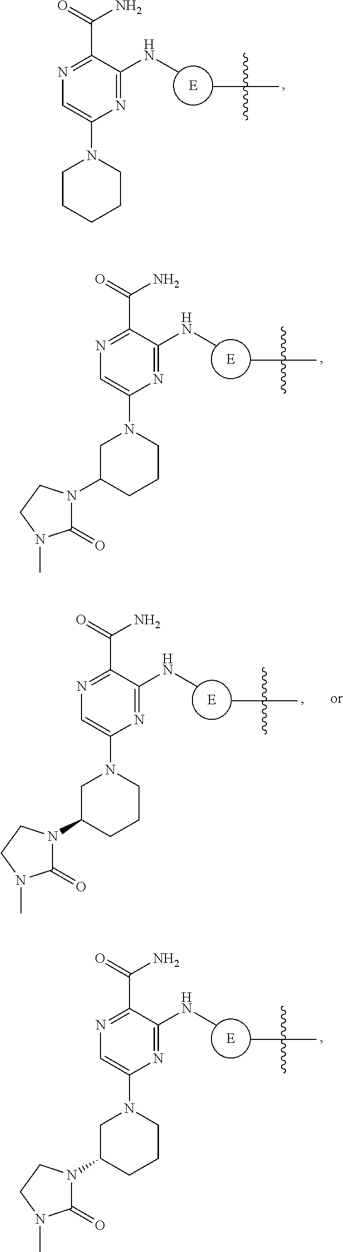

wherein
T is selected from the group consisting of CH and N, preferably selected from CH;
R is selected from the group consisting of hydrogen and 5- to 6-membered heterocycloalkyl, wherein the heterocycloalkyl is optionally substituted with ═O or C₁₋₆ alkyl; preferably, R is selected from the group consisting of hydrogen, imidazolidinonyl, and imidazolidinonyl substituted with C₁₋₄ alkyl; more preferably, R is selected from the group consisting of hydrogen, imidazolidinonyl, 1-methyl-imidazolidinonyl, 1-ethyl-imidazolidinonyl, and 1-propyl-imidazolidinonyl;
ring E is selected from the group consisting of phenyl, benzocycloalkenyl, and benzoheterocycloalkenyl; preferably, ring E is selected from the group consisting of phenyl, benzopiperidinyl, benzodihydropyrrolyl, spiro[benzopyran-piperidine], and benzodihydrooxazinopiperazine;
Cy¹ is selected from the group consisting of a bond and the following groups optionally substituted with one or more R^{a}: C₆₋₉ cycloalkyl or 5- to 11-membered heterocycloalkyl containing 1-3 heteroatoms selected from the group consisting of N, O, and S (e.g., 1-3 or 1-2 heteroatoms selected from the group consisting of N and O);
LNK is selected from the group consisting of a bond and C₁₋₄ alkylene; preferably, LNK is selected from the group consisting of a bond and C₁₋₃ alkylene;
Cy² is absent or selected from the group consisting of C₄₋₆ cycloalkyl and 4- to 6-membered heterocycloalkyl, wherein the cycloalkyl or heterocycloalkyl is optionally substituted with one or more R^{b}; preferably, Cy² is absent or selected from the group consisting of C₄₋₆ cycloalkyl and 4- to 6-membered heterocycloalkyl, wherein the heterocycloalkyl comprises 1-2 heteroatoms selected from the group consisting of N and O.

In some specific embodiments, the present application relates to a compound of formula I, a stereoisomer thereof, or a pharmaceutically acceptable salt thereof, wherein
ring A is absent or selected from the group consisting of cyclopentenyl, dihydropyrrolyl, tetrahydropyridinyl, tetrahydroazepinyl, and phenyl; preferably, ring A is selected from the group consisting of cyclopentenyl, dihydropyrrolyl, tetrahydropyridinyl, and tetrahydroazepinyl, and more preferably, ring A is selected from the group consisting of dihydropyrrolyl, tetrahydropyridinyl, and tetrahydroazepinyl;
preferably, the structural moiety

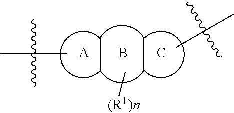

is selected from the group consisting of

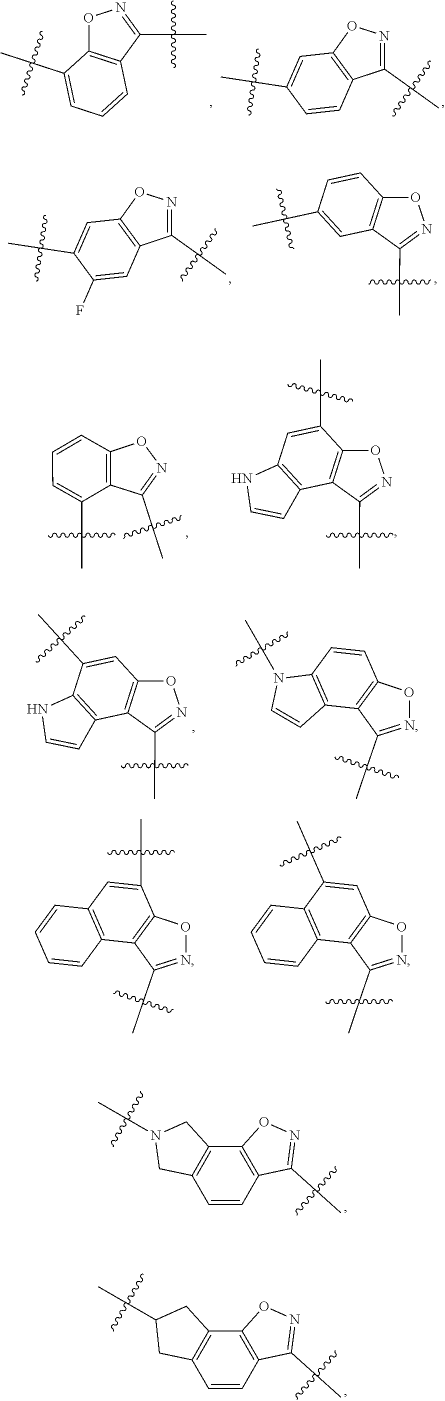

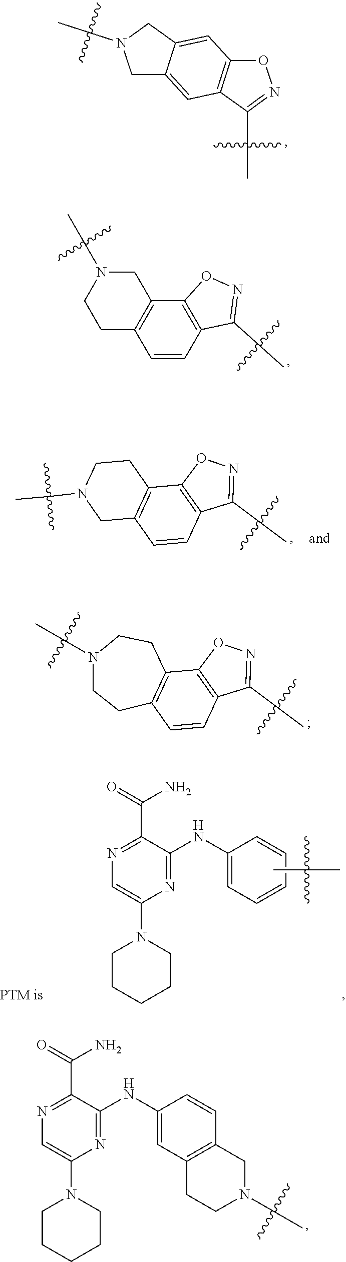

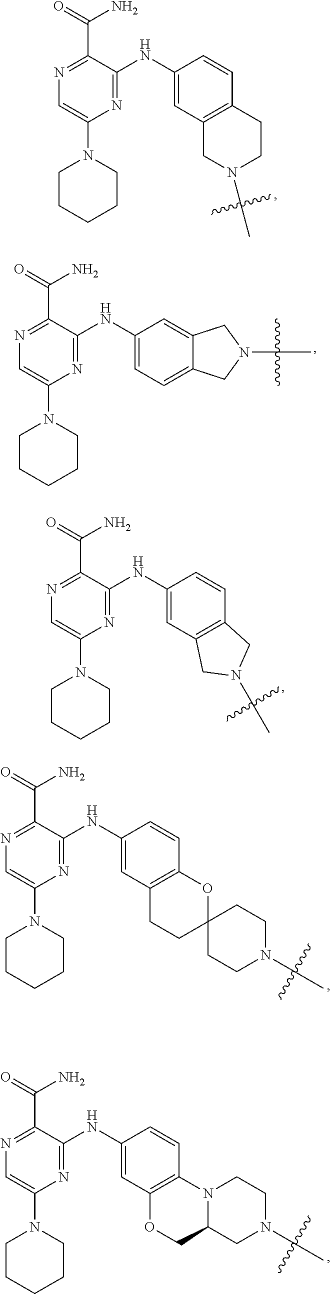

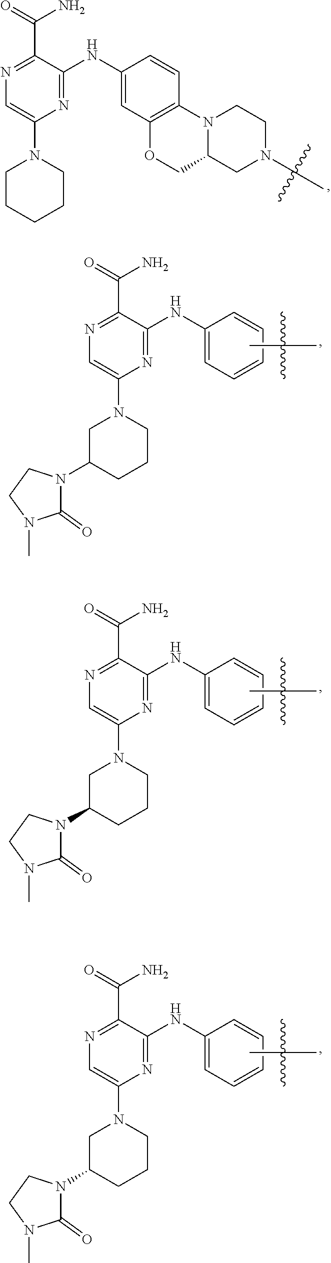

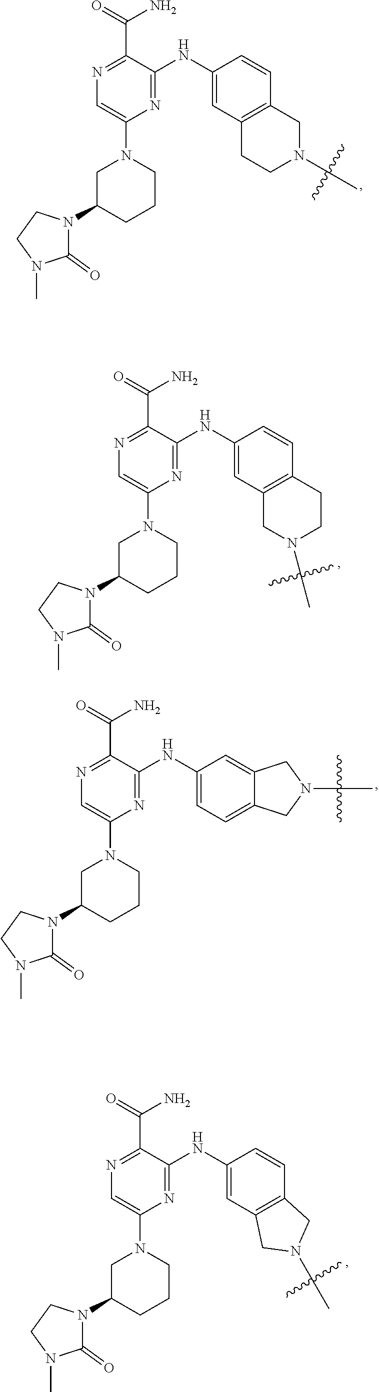

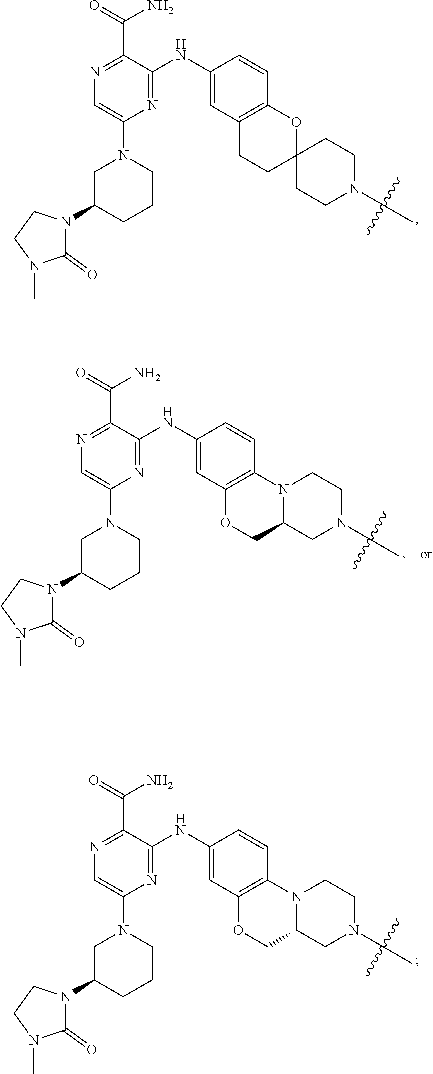 [SMILES: *N1CCC2(CCc3cc(Nc4nc(N5CCC[C@@H](N6CCN(C)C6=O)C5)cnc4C(N)=O)ccc3O2)CC1 ; *N1CCN2c3ccc(Nc4nc(N5CCC[C@@H](N6CCN(C)C6=O)C5)cnc4C(N)=O)cc3OC[C@@H]2C1 ; *N1CCN2c3ccc(Nc4nc(N5CCC[C@@H](N6CCN(C)C6=O)C5)cnc4C(N)=O)cc3OC[C@H]2C1]

preferably, PTM is

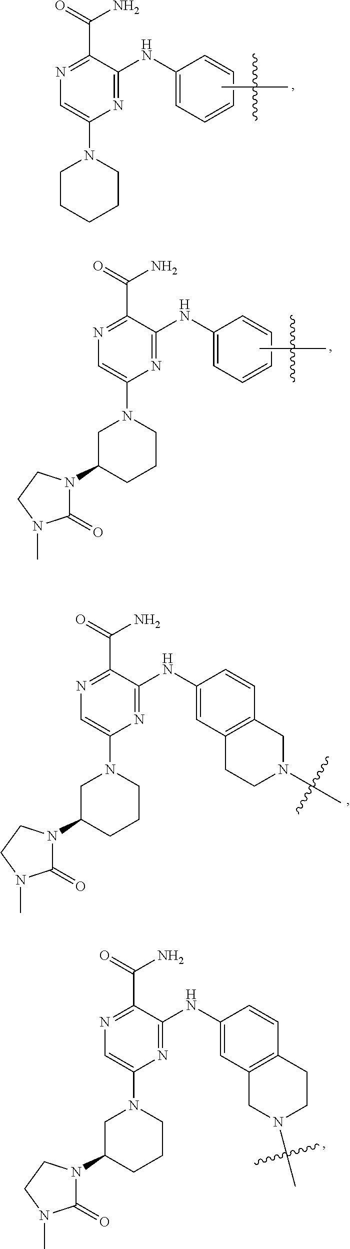

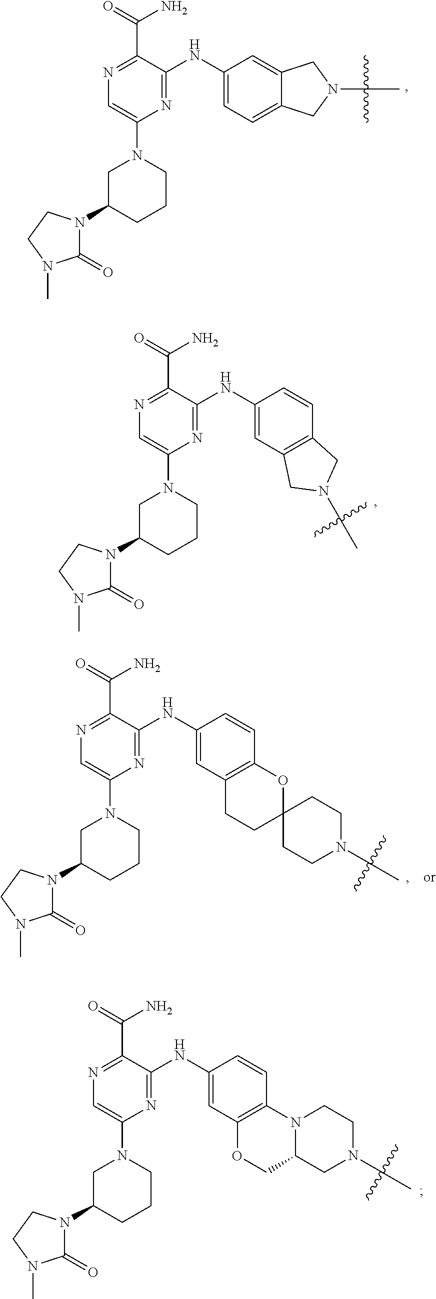

Cy¹ is selected from the group consisting of a bond, piperidinyl, cyclohexyl, spirononanyl, monoazaspirononany 1, octahydrocyclopentapyrrolyl, monoazaspiroundecanyl, monooxamonoazaspiroundecanyl, and azabicyclononanyl; preferably, Cy¹ is selected from the group consisting of a bond,

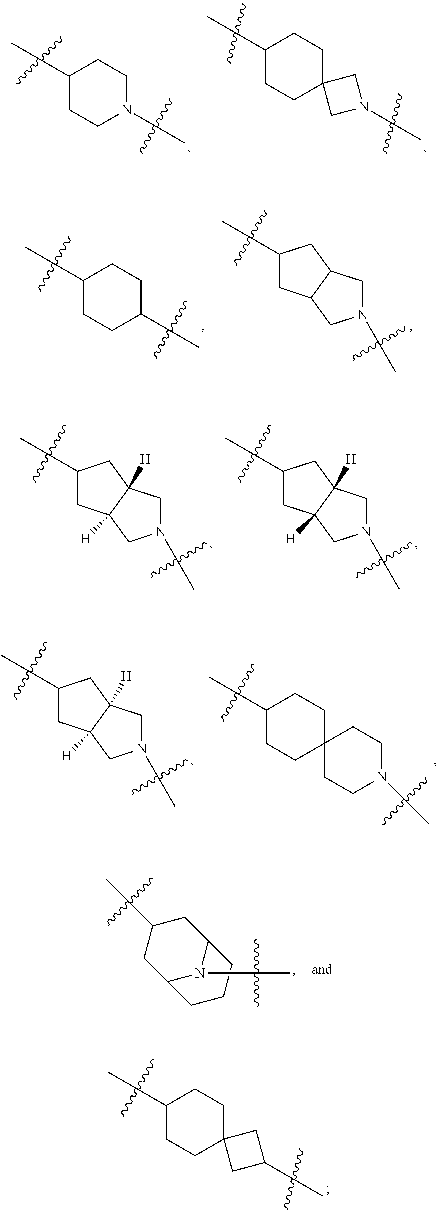

LNK is selected from the group consisting of a bond and C₁₋₃ alkylene; preferably, LNK is selected from the group consisting of a bond and —CH₂—;

In some embodiments, Cy² is absent or selected from the group consisting of cyclobutyl, cyclopentyl, cyclohexyl, azetidinyl, pyrrolidinyl, and piperidinyl, wherein the cyclobutyl, cyclopentyl, azetidinyl, pyrrolidinyl, or piperidinyl is optionally substituted with one or more R^{b}; preferably, Cy² is absent or selected from the group consisting of

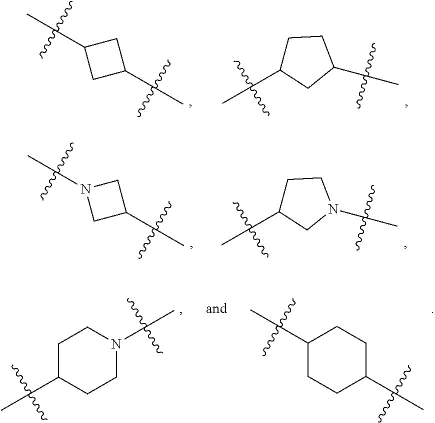 [SMILES: *C1CC(*)C1 ; *C1CCC(*)C1 ; *C1CN(*)C1 ; *C1CCN(*)C1 ; *C1CCN(*)CC1 ; *C1CCC(*)CC1]

In some embodiments, the heteroatom of the heterocycloalkenyl or heterocycloalkyl is selected from the group consisting of N, NH, O, and S. In some embodiments, the heteroatom of the heterocycloalkenyl or heterocycloalkyl is selected from the group consisting of N, O, and S. In some embodiments, the heteroatom of the heteroaryl is selected from the group consisting of N, O, and S. In some embodiments, the heteroatom of the heteroalkylene is selected from the group consisting of N, NH, O, S, S(O), and S(O)₂.

In some embodiments, the number of the heteroatom of the heterocycloalkenyl, heterocycloalkyl, heteroalkylene, or heteroaryl is selected from the group consisting of 1, 2, 3, 4, 5, and 6. In some embodiments, the number of the heteroatom of the heterocycloalkenyl, heterocycloalkyl, heteroalkylene, or heteroaryl is selected from the group consisting of 1, 2, 3, and 4. In some embodiments, the number of the heteroatom of the heterocycloalkenyl, heterocycloalkyl, heteroalkylene, or heteroaryl is selected from the group consisting of 1, 2, and 3.

The present application also relates to compounds of formula I′-1A, formula I′-2A, formula I′-1A-1, formula I′-2A-1, formula I′-3A-1, formula I′-3A-2, formula I′-4A-1, and formula I′-4A-2, stereoisomers thereof, or pharmaceutically acceptable salts thereof,

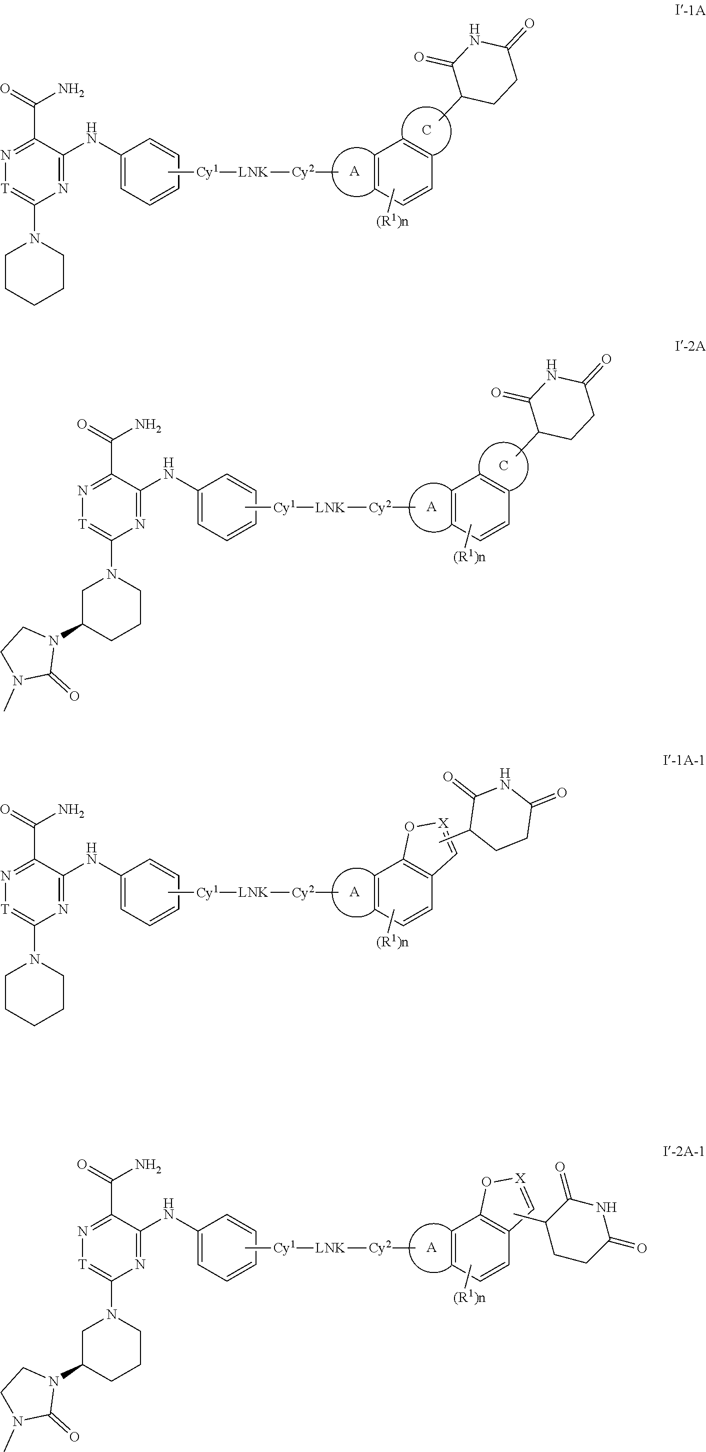

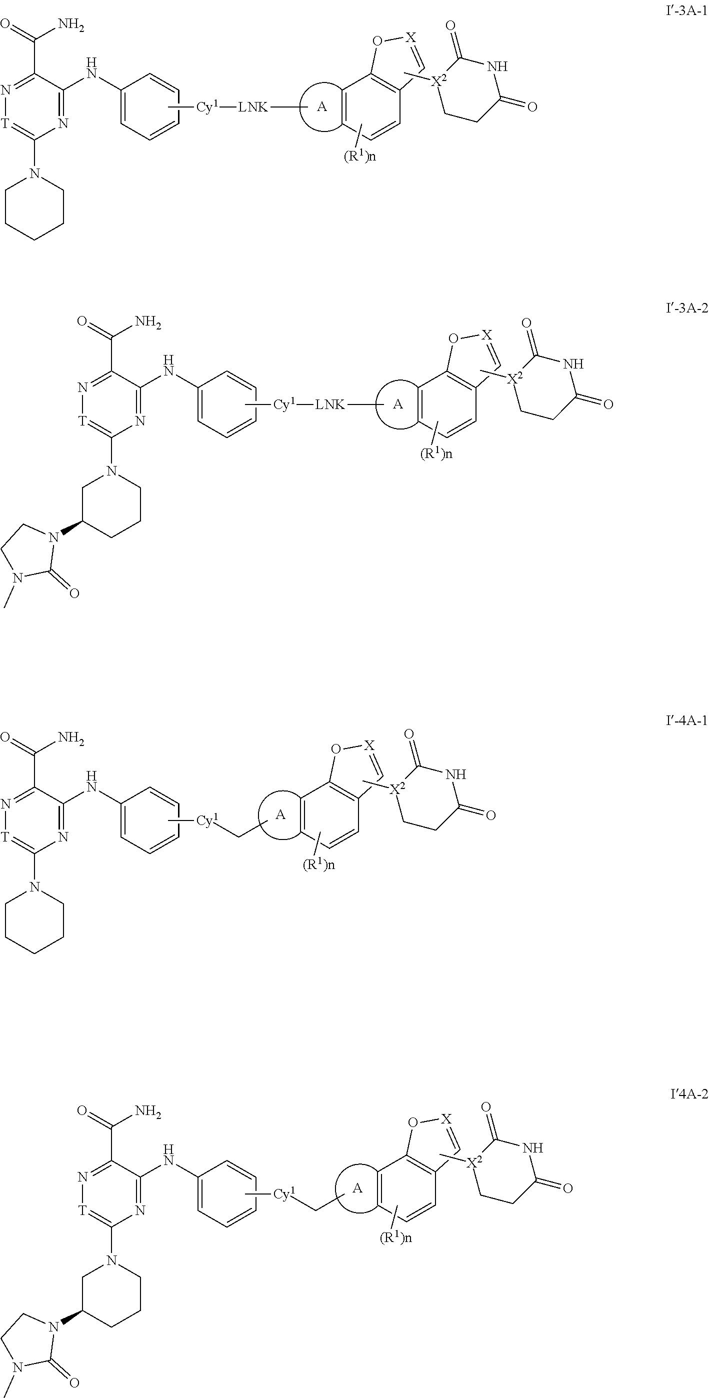

wherein ring A, ring C, Cy¹, Cy², LNK, R¹, n, T, and X² are as defined herein;
X is selected from the group consisting of CH and N.

In some embodiments, the structural moiety

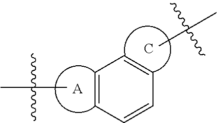

is selected from the group consisting of

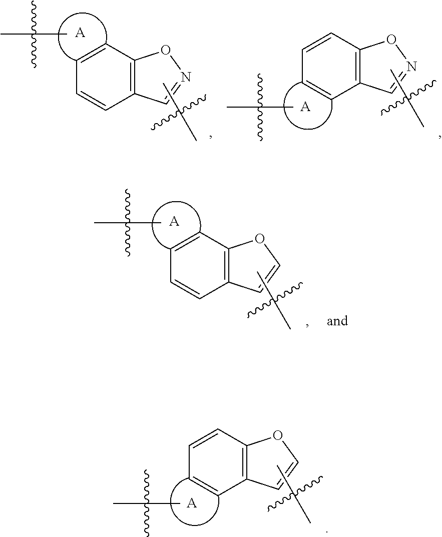

In some embodiments, the structural moiety

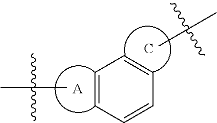

is selected from the group consisting of

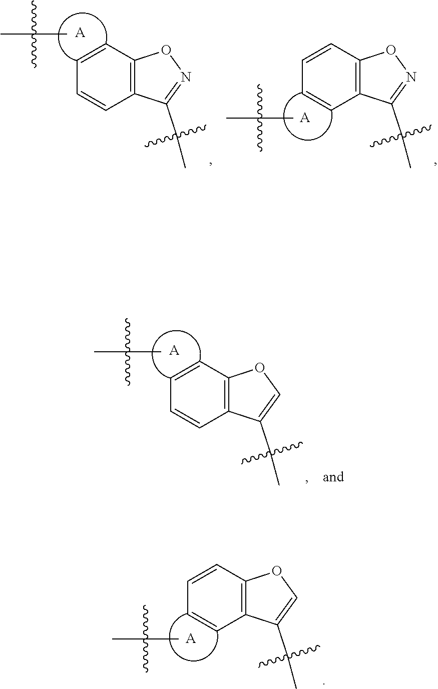

In some embodiments, the structural moiety

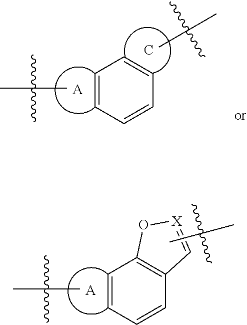

is selected from the group consisting of

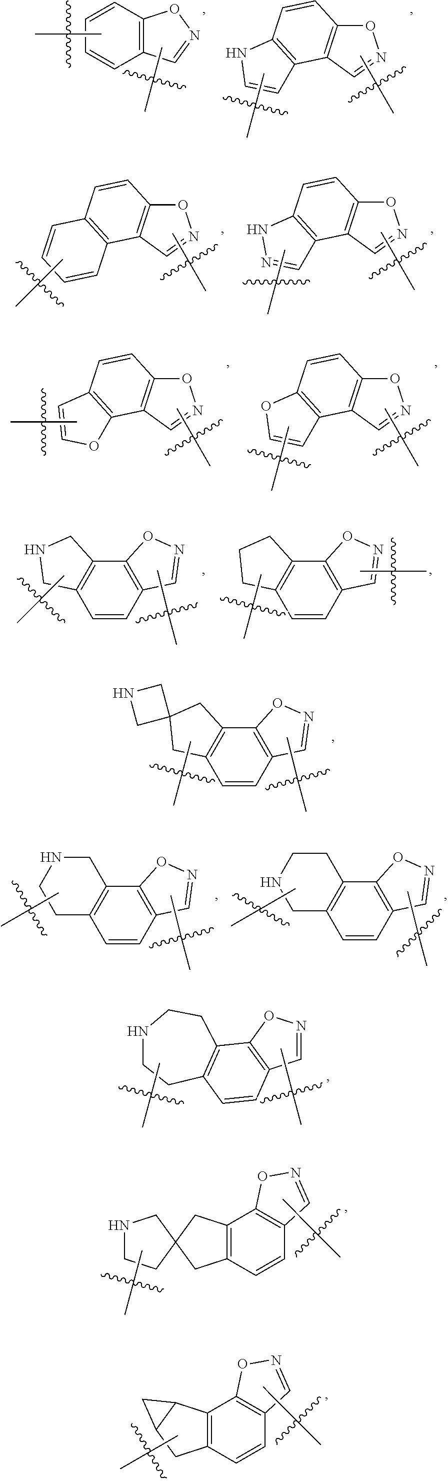

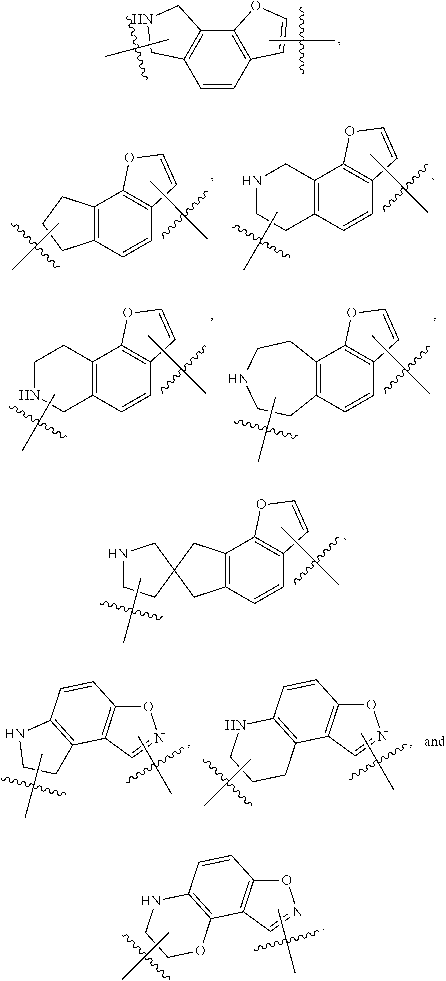

In some specific embodiments, the structural moiety

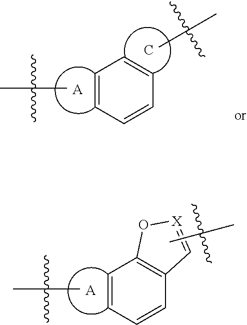

is selected from the group consisting of

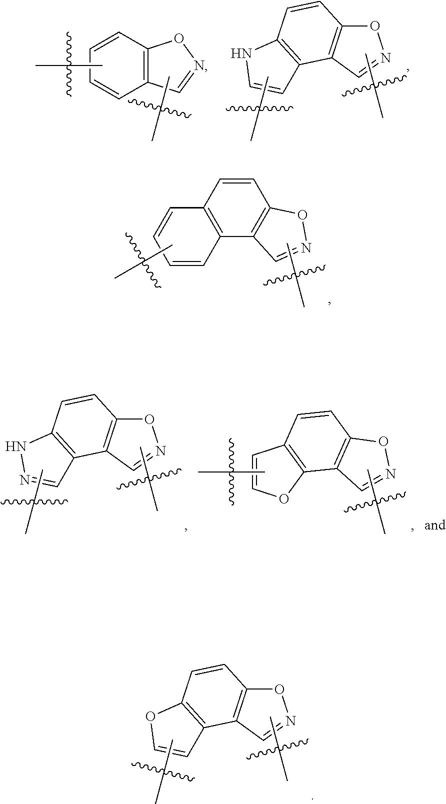

In some specific embodiments, the structural moiety

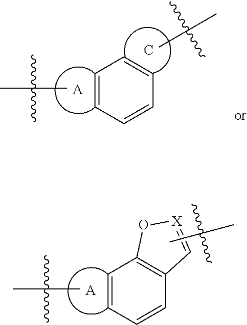

is selected from the group consisting of

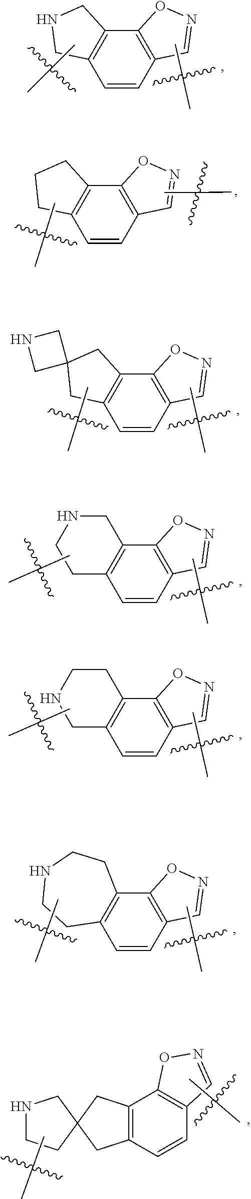

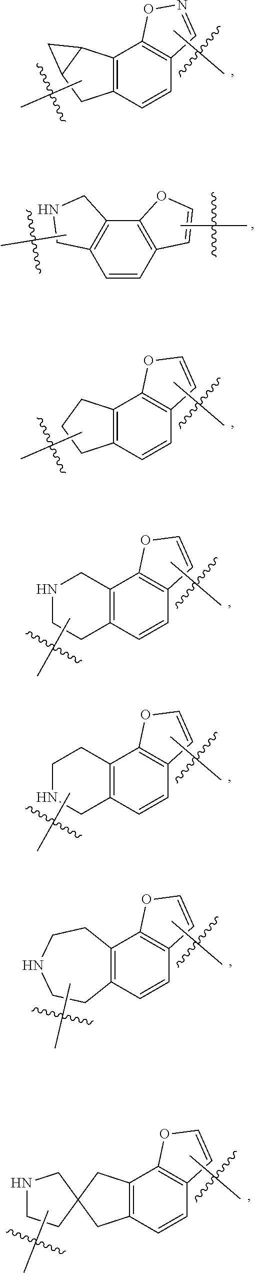

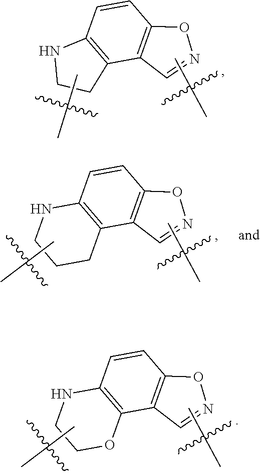

In some embodiments, the structural moiety

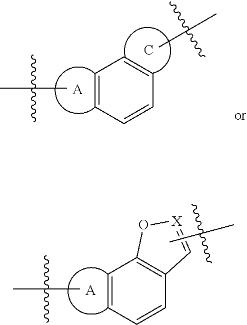

is selected from the group consisting of

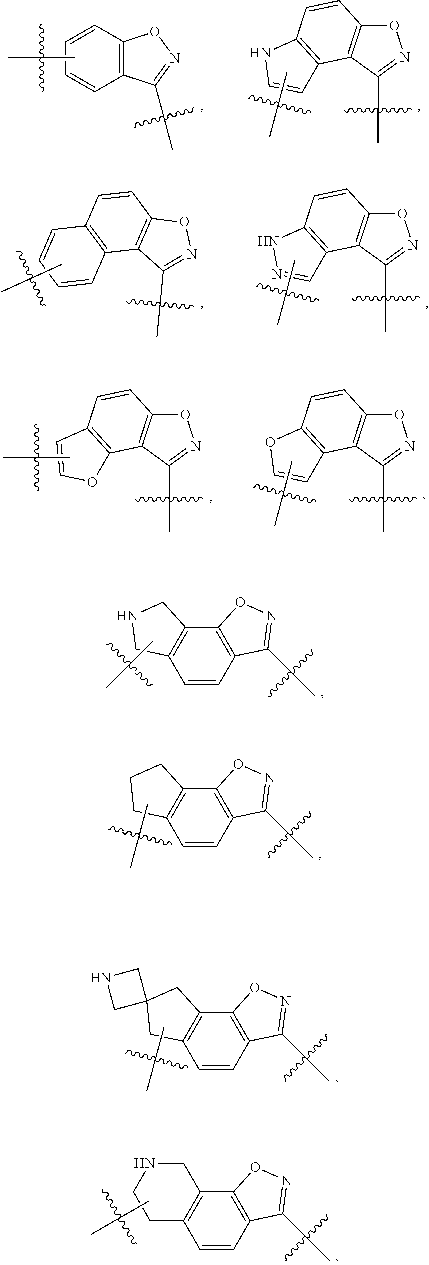

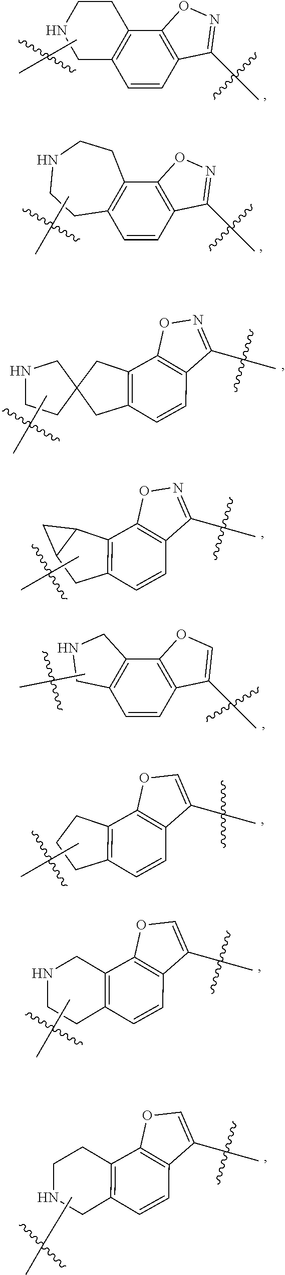

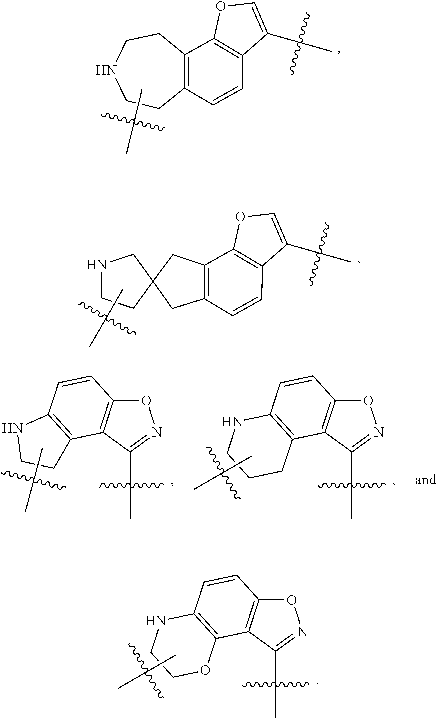

In some specific embodiments, the structural moiety

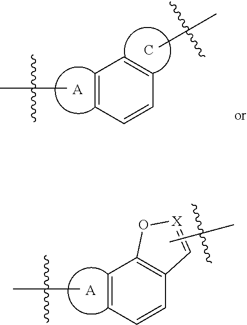

is selected from the group consisting of

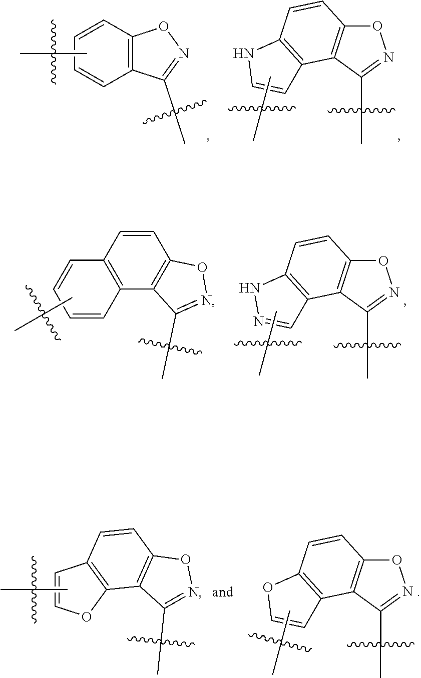

In some specific embodiments, the structural moiety

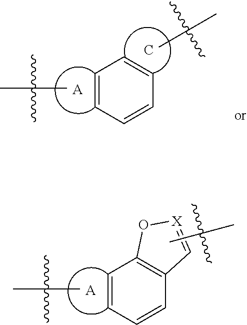

is selected from the group consisting of

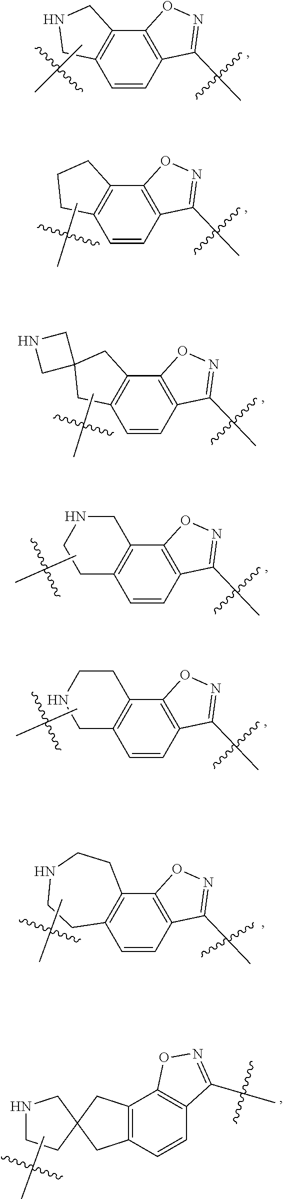

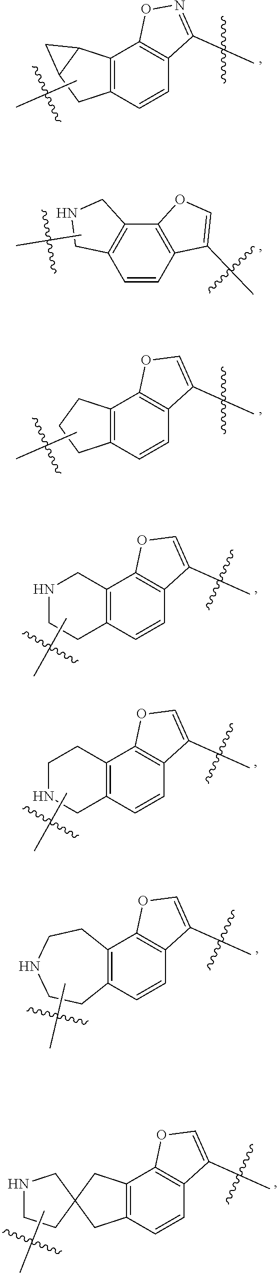

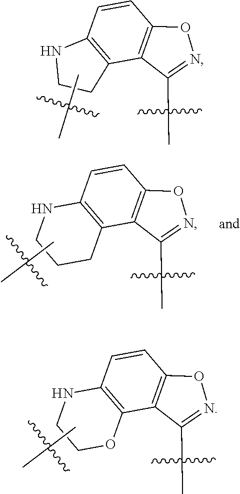

In some embodiments, the structural moiety

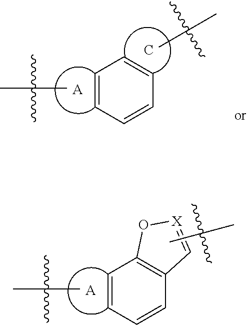

is selected from the group consisting of

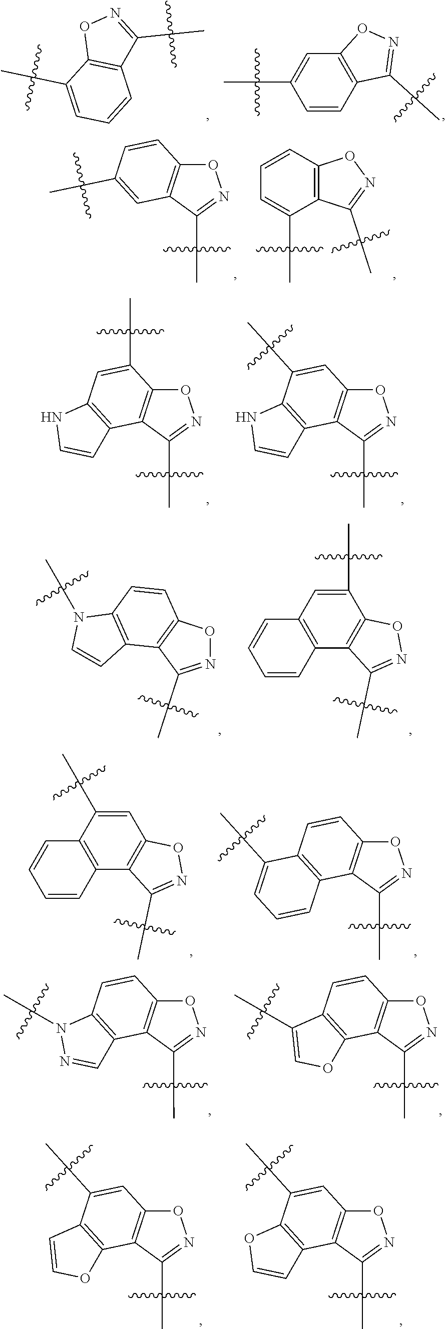

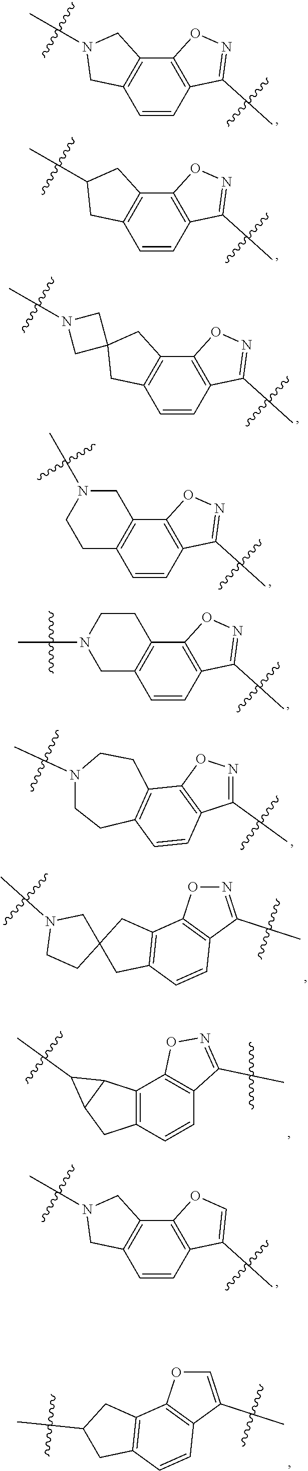

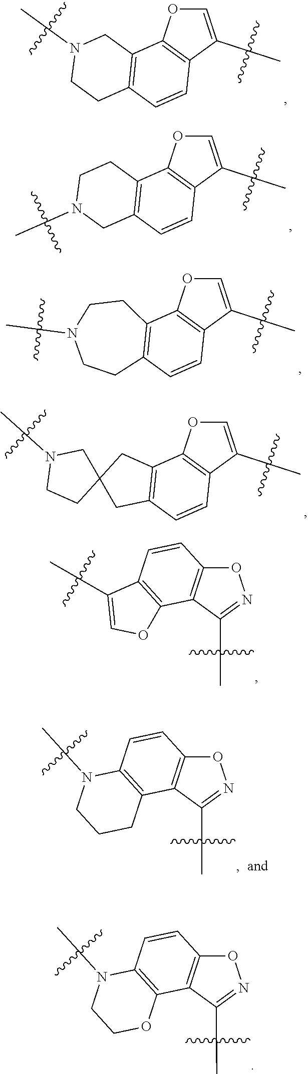

In some specific embodiments, the structural moiety

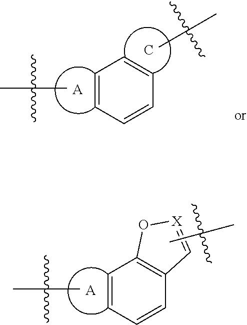

is selected from the group consisting of

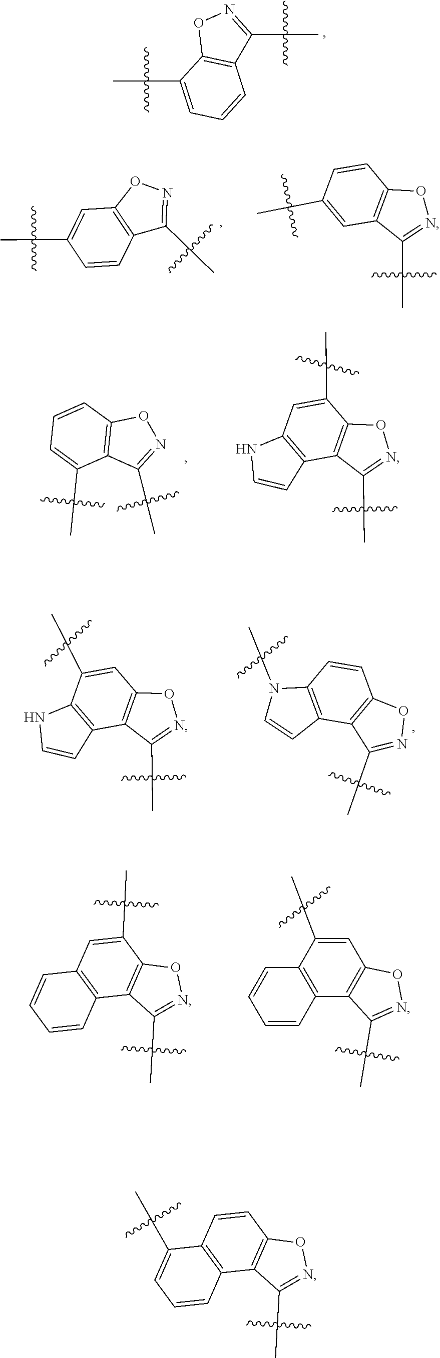

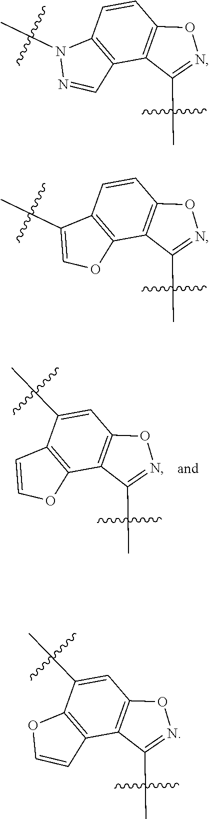 [SMILES: *c1noc2ccc3c(cnn3*)c12 ; *c1coc2c1ccc1onc(*)c12 ; *c1cc2onc(*)c2c2occc12 ; *c1cc2onc(*)c2c2ccoc12]

In some specific embodiments, the structural moiety

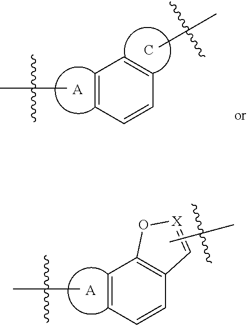

is selected from the group consisting of

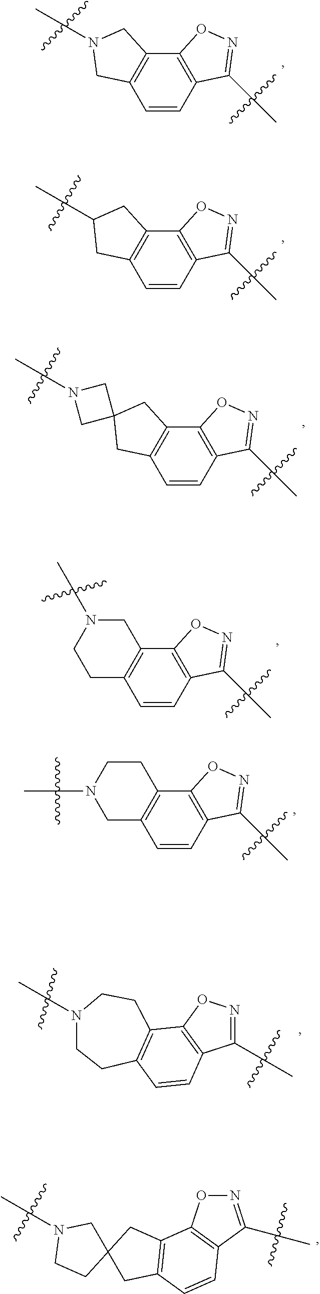

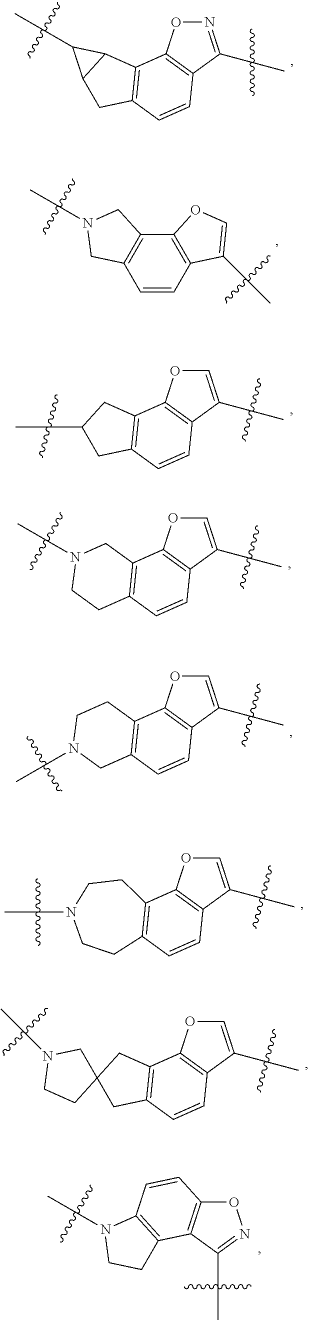

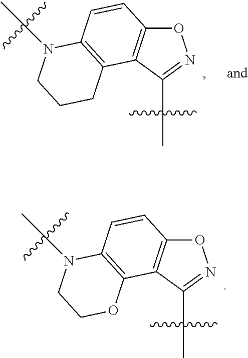 [SMILES: *c1noc2ccc3c(c12)CCCN3* ; *c1noc2ccc3c(c12)OCCN3*]

In some specific embodiments, the structural moiety

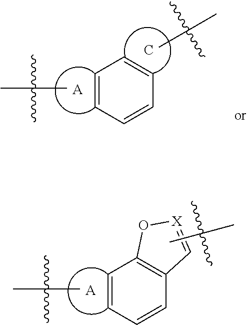

is selected from the group consisting of

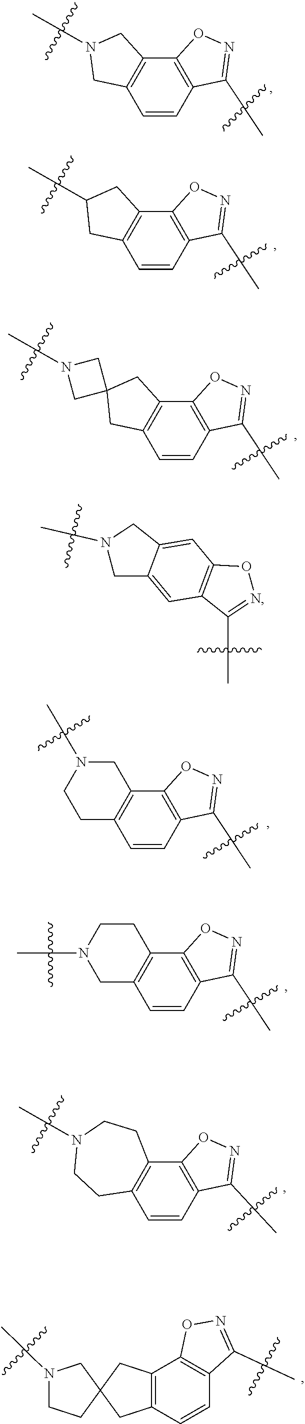

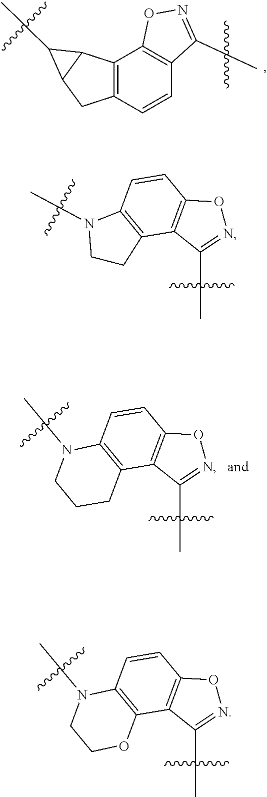 [SMILES: *c1noc2c3c(ccc12)CC1C(*)C31 ; *c1noc2ccc3c(c12)CCN3* ; *c1noc2ccc3c(c12)CCCN3* ; *c1noc2ccc3c(c12)OCCN3*]

In some other embodiments, the structural moiety

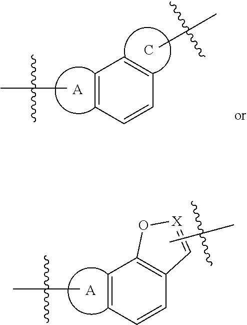

is selected from the group consisting of

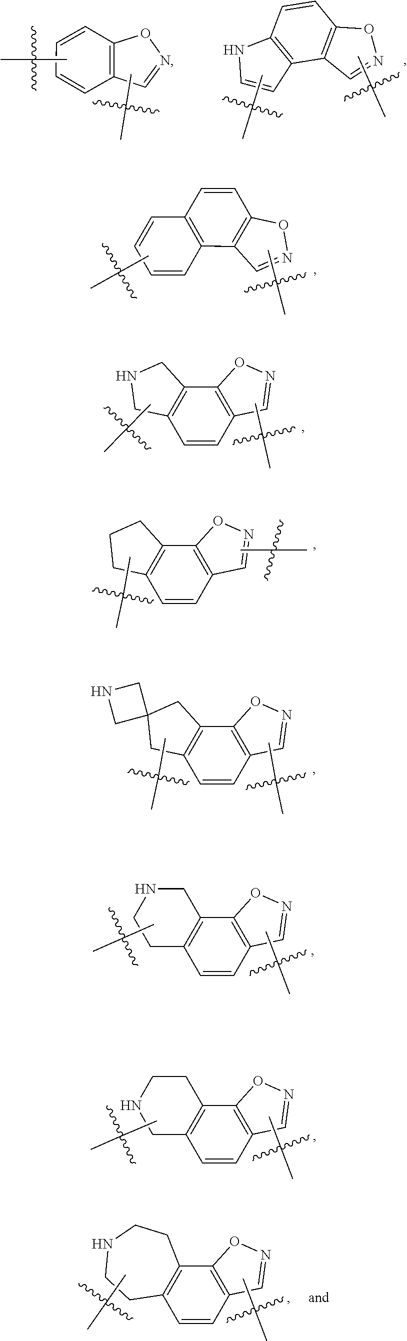

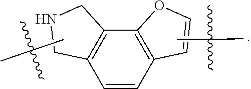

In some other embodiments, the structural moiety

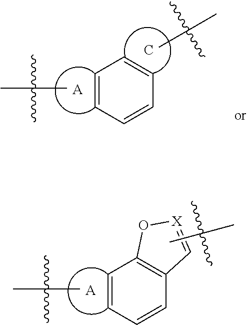

is selected from the group consisting of

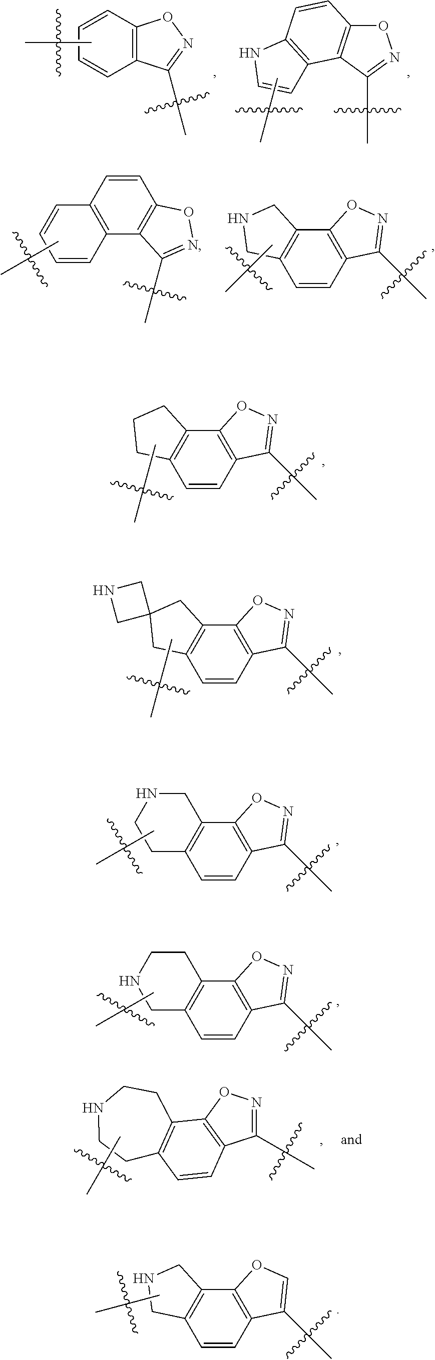

In some other embodiments, the structural moiety

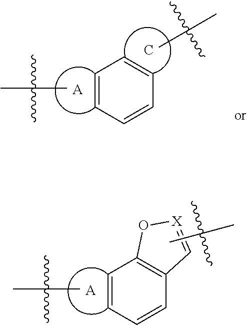

is selected from the group consisting of

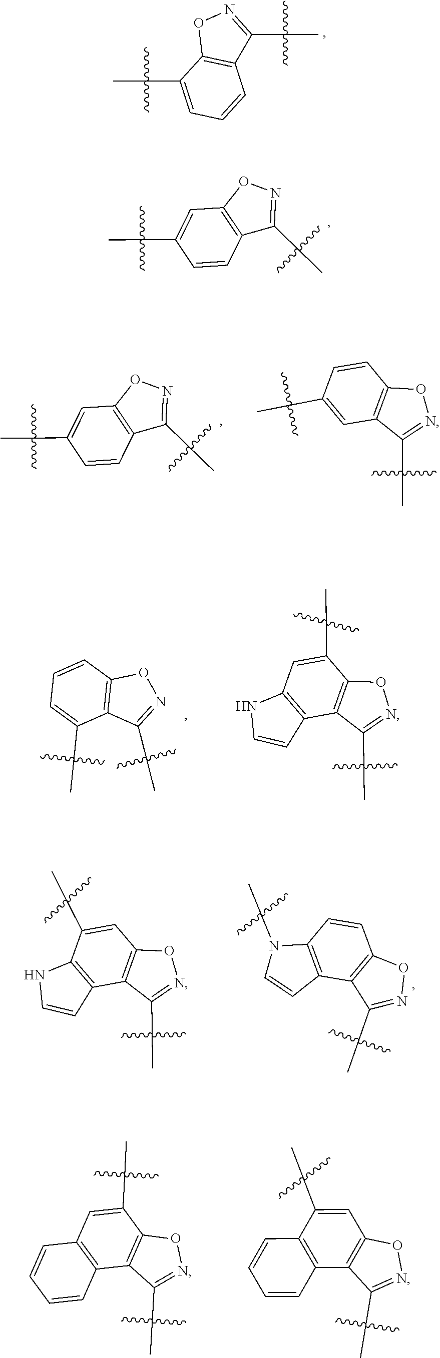

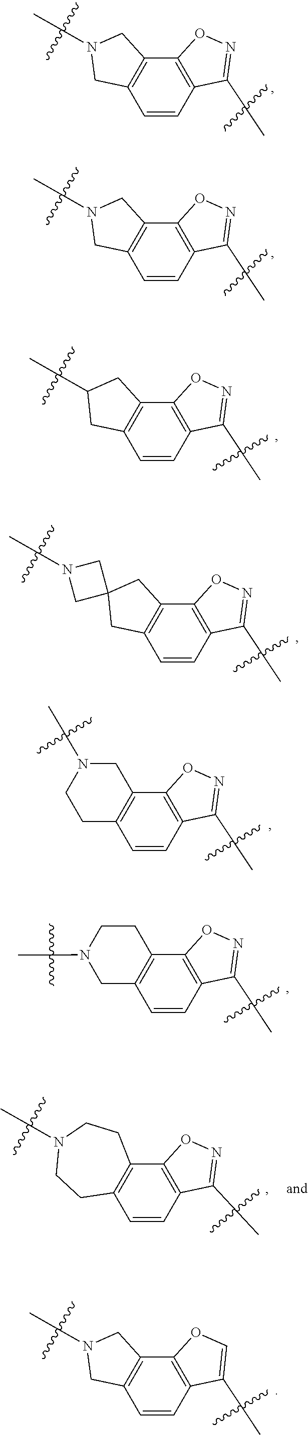

In some embodiments, the structural moiety -Cy¹-LNK-, -LNK-Cy²-, -Cy¹-Cy²-, or -Cy¹-LNK-Cy²- are as described herein.

The present application also relates to a compound of a formula selected from the group consisting of the following formulas, a stereoisomer thereof, and a pharmaceutically acceptable salt thereof:

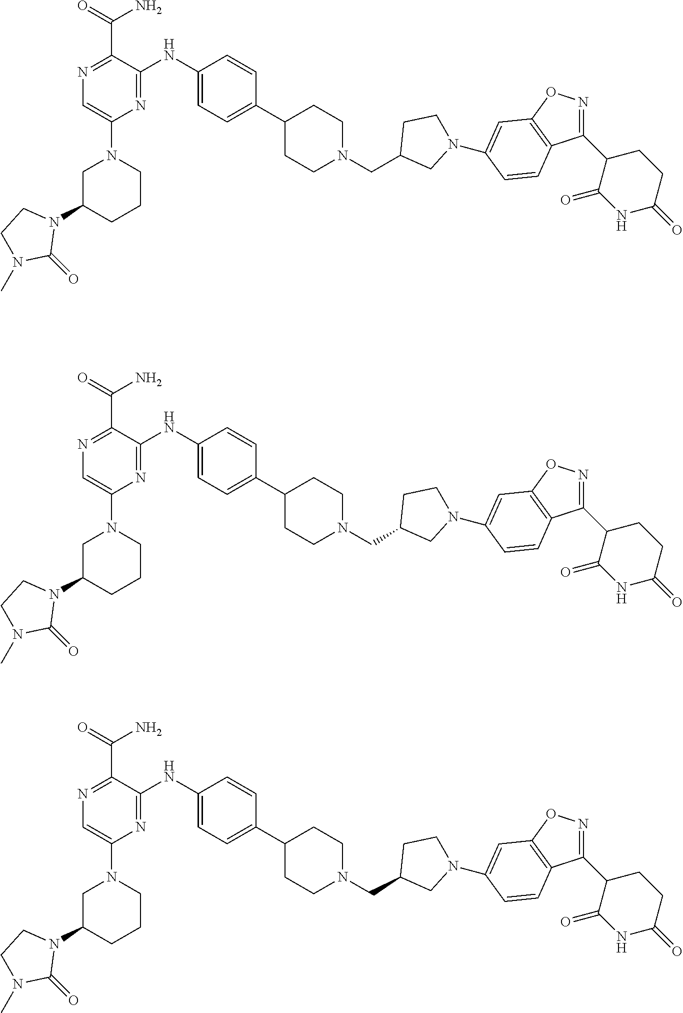 [SMILES: CN1CCN([C@@H]2CCCN(c3cnc(C(N)=O)c(Nc4ccc(C5CCN(CC6CCN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@@H]6CCN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@H]6CCN(c7ccc8c(C9CCC(=O)NC9=O)noc8c7)C6)CC5)cc4)n3)C2)C1=O]

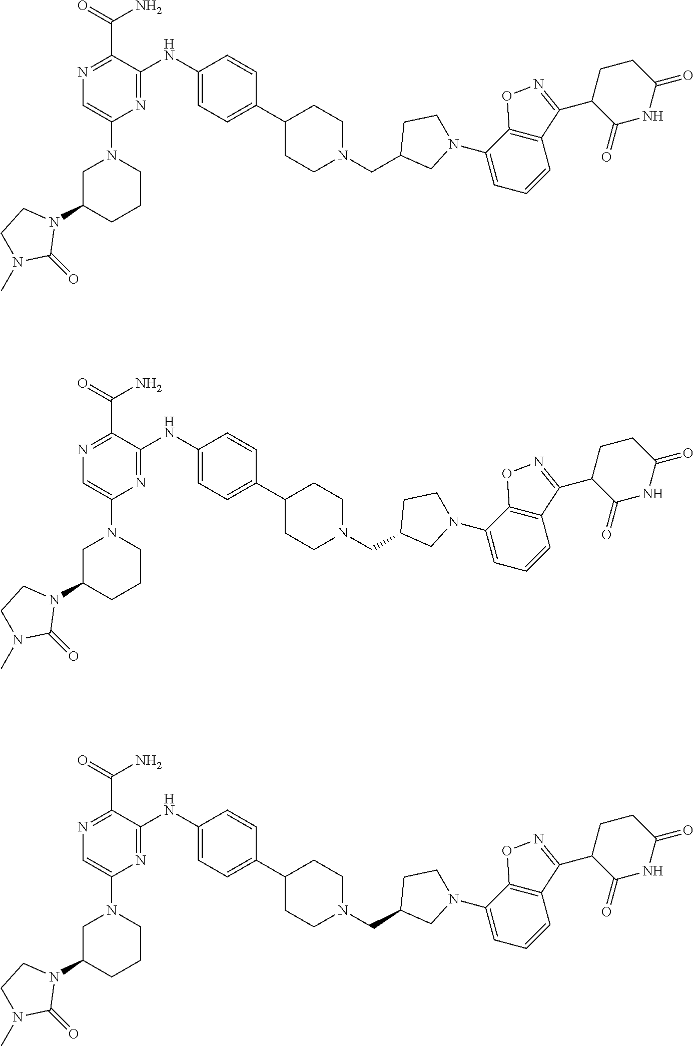 [SMILES: CN1CCN([C@@H]2CCCN(c3cnc(C(N)=O)c(Nc4ccc(C5CCN(CC6CCN(c7cccc8c(C9CCC(=O)NC9=O)noc78)C6)CC5)cc4)n3)C2)C1=O ; CN1CCN([C@@H]2CCCN(c3cnc(C(N)=O)c(Nc4ccc(C5CCN(C[C@@H]6CCN(c7cccc8c(C9CCC(=O)NC9=O)noc78)C6)CC5)cc4)n3)C2)C1=O ; CN1CCN([C@@H]2CCCN(c3cnc(C(N)=O)c(Nc4ccc(C5CCN(C[C@H]6CCN(c7cccc8c(C9CCC(=O)NC9=O)noc78)C6)CC5)cc4)n3)C2)C1=O]

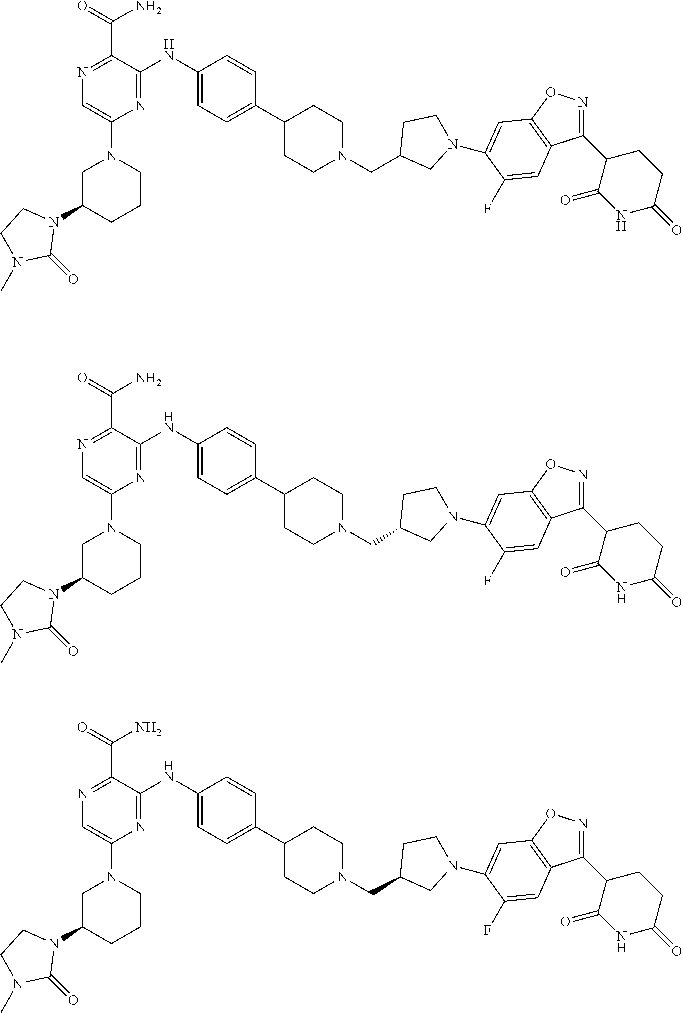 [SMILES: CN1CCN([C@@H]2CCCN(c3cnc(C(N)=O)c(Nc4ccc(C5CCN(CC6CCN(c7cc8onc(C9CCC(=O)NC9=O)c8cc7F)C6)CC5)cc4)n3)C2)C1=O ; CN1CCN([C@@H]2CCCN(c3cnc(C(N)=O)c(Nc4ccc(C5CCN(C[C@@H]6CCN(c7cc8onc(C9CCC(=O)NC9=O)c8cc7F)C6)CC5)cc4)n3)C2)C1=O ; CN1CCN([C@@H]2CCCN(c3cnc(C(N)=O)c(Nc4ccc(C5CCN(C[C@H]6CCN(c7cc8onc(C9CCC(=O)NC9=O)c8cc7F)C6)CC5)cc4)n3)C2)C1=O]

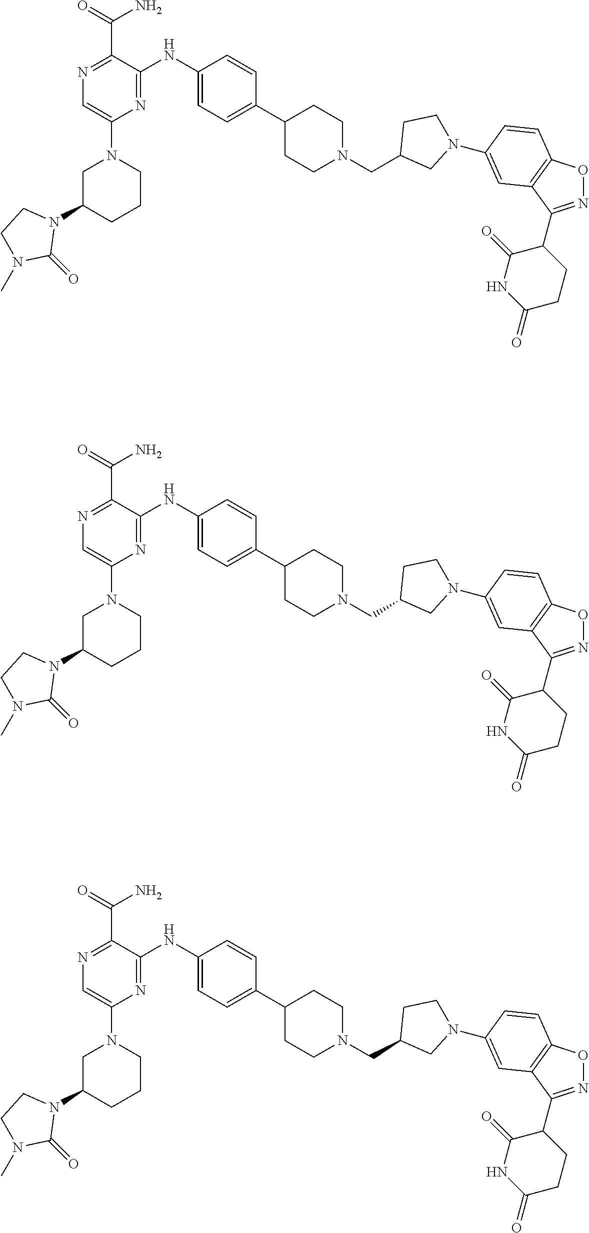 [SMILES: CN1CCN([C@@H]2CCCN(c3cnc(C(N)=O)c(Nc4ccc(C5CCN(CC6CCN(c7ccc8onc(C9CCC(=O)NC9=O)c8c7)C6)CC5)cc4)n3)C2)C1=O ; CN1CCN([C@@H]2CCCN(c3cnc(C(N)=O)c(Nc4ccc(C5CCN(C[C@@H]6CCN(c7ccc8onc(C9CCC(=O)NC9=O)c8c7)C6)CC5)cc4)n3)C2)C1=O ; CN1CCN([C@@H]2CCCN(c3cnc(C(N)=O)c(Nc4ccc(C5CCN(C[C@H]6CCN(c7ccc8onc(C9CCC(=O)NC9=O)c8c7)C6)CC5)cc4)n3)C2)C1=O]

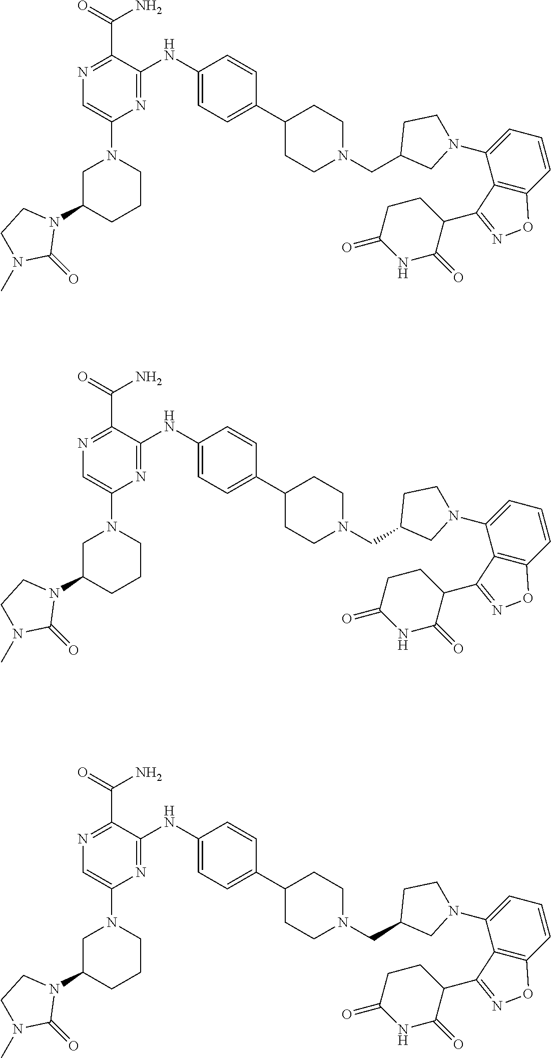 [SMILES: CN1CCN([C@@H]2CCCN(c3cnc(C(N)=O)c(Nc4ccc(C5CCN(CC6CCN(c7cccc8onc(C9CCC(=O)NC9=O)c78)C6)CC5)cc4)n3)C2)C1=O ; CN1CCN([C@@H]2CCCN(c3cnc(C(N)=O)c(Nc4ccc(C5CCN(C[C@@H]6CCN(c7cccc8onc(C9CCC(=O)NC9=O)c78)C6)CC5)cc4)n3)C2)C1=O ; CN1CCN([C@@H]2CCCN(c3cnc(C(N)=O)c(Nc4ccc(C5CCN(C[C@H]6CCN(c7cccc8onc(C9CCC(=O)NC9=O)c78)C6)CC5)cc4)n3)C2)C1=O]

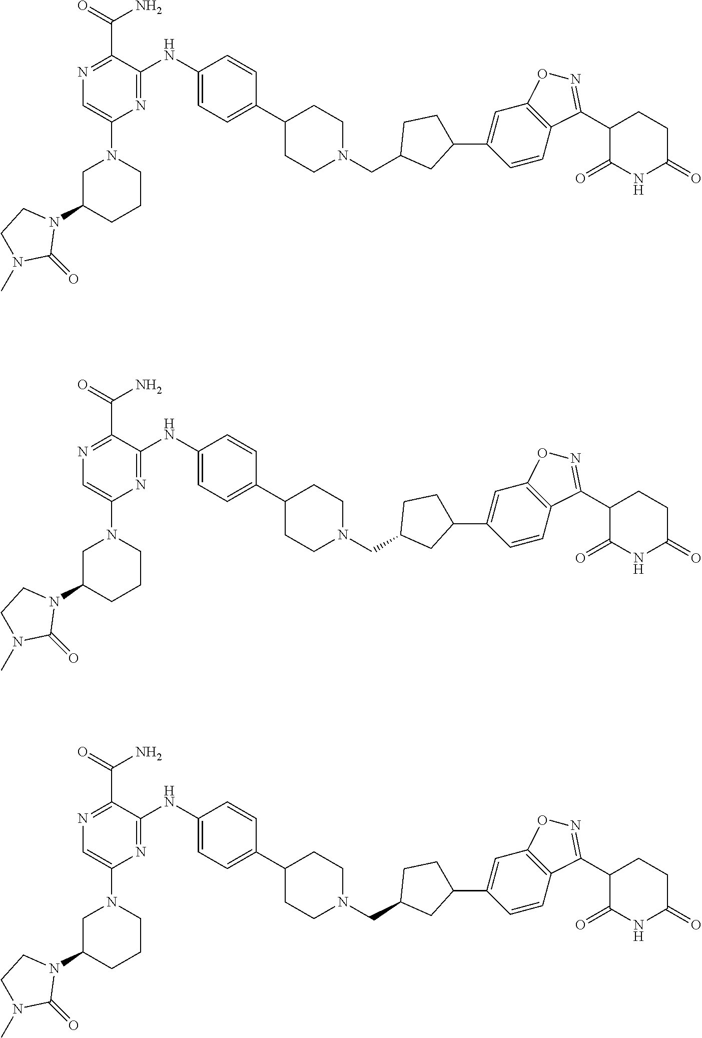 [SMILES: CN1CCN([C@@H]2CCCN(c3cnc(C(N)=O)c(Nc4ccc(C5CCN(CC6CCC(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@@H]6CCC(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@H]6CCC(c7ccc8c(C9CCC(=O)NC9=O)noc8c7)C6)CC5)cc4)n3)C2)C1=O]

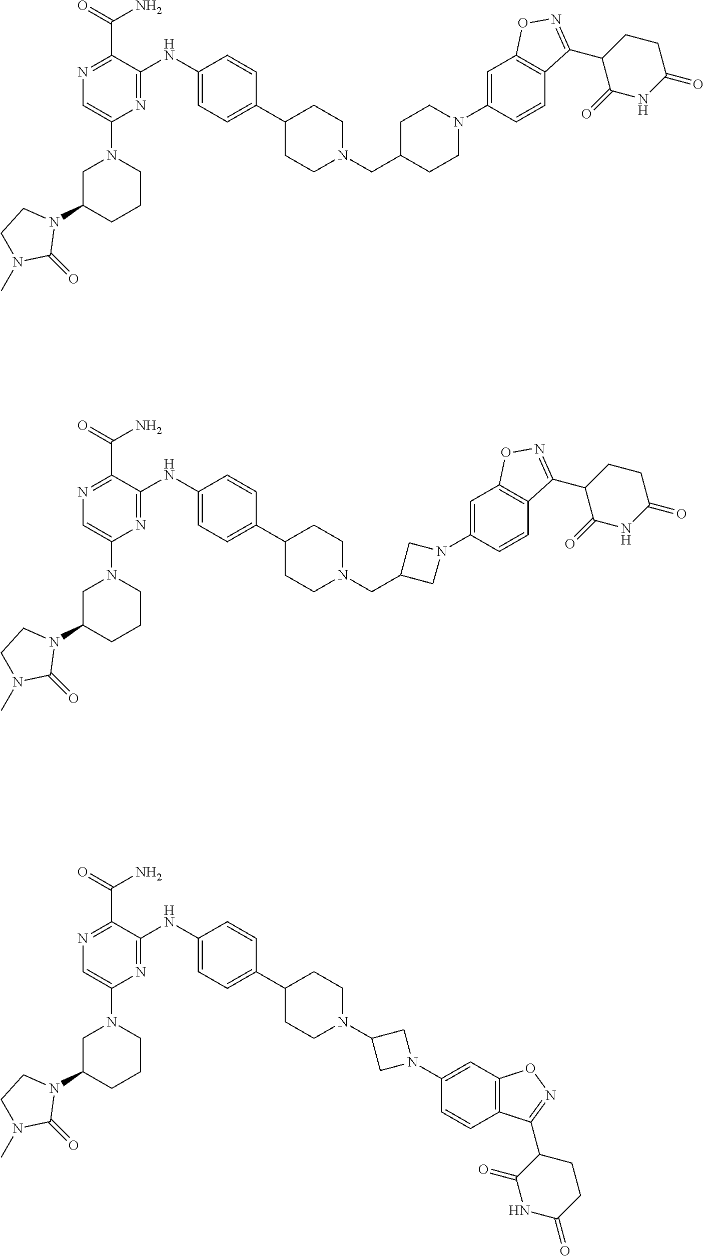 [SMILES: CN1CCN([C@@H]2CCCN(c3cnc(C(N)=O)c(Nc4ccc(C5CCN(CC6CCN(c7ccc8c(C9CCC(=O)NC9=O)noc8c7)CC6)CC5)cc4)n3)C2)C1=O ; CN1CCN([C@@H]2CCCN(c3cnc(C(N)=O)c(Nc4ccc(C5CCN(CC6CN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C6CN(c7ccc8c(C9CCC(=O)NC9=O)noc8c7)C6)CC5)cc4)n3)C2)C1=O]

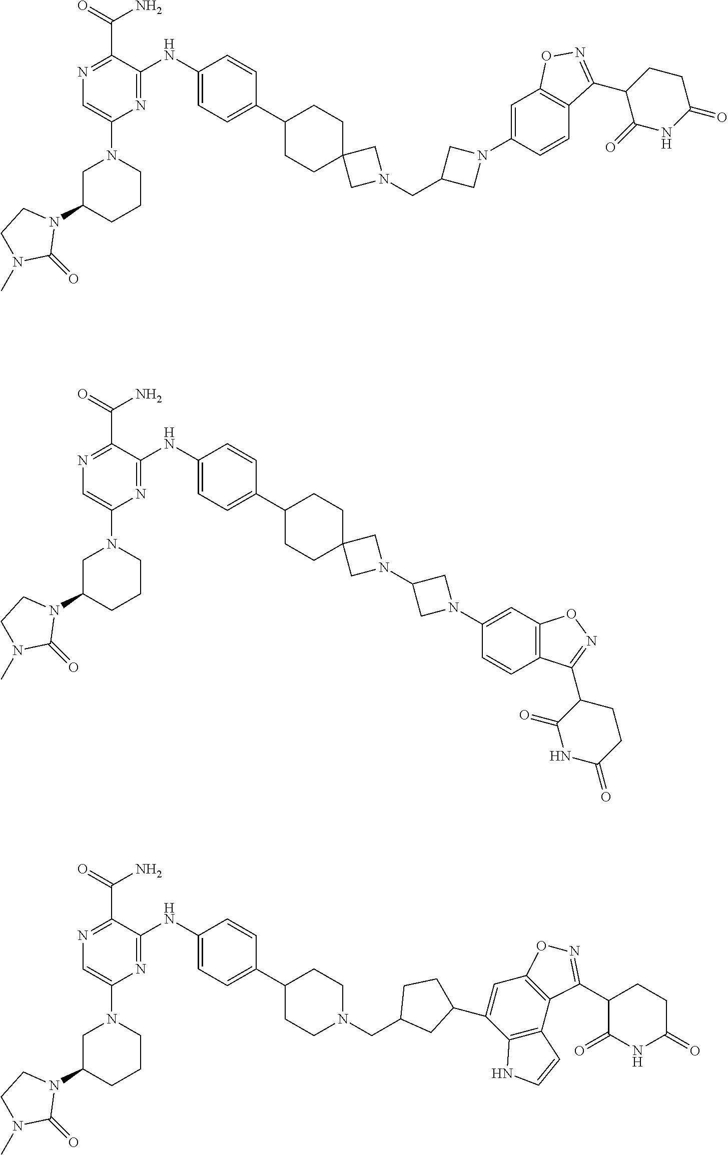 [SMILES: CN1CCN([C@@H]2CCCN(c3cnc(C(N)=O)c(Nc4ccc(C5CCC6(CC5)CN(CC5CN(c7ccc8c(C9CCC(=O)NC9=O)noc8c7)C5)C6)cc4)n3)C2)C1=O ; CN1CCN([C@@H]2CCCN(c3cnc(C(N)=O)c(Nc4ccc(C5CCC6(CC5)CN(C5CN(c7ccc8c(C9CCC(=O)NC9=O)noc8c7)C5)C6)cc4)n3)C2)C1=O ; CN1CCN([C@@H]2CCCN(c3cnc(C(N)=O)c(Nc4ccc(C5CCN(CC6CCC(c7cc8onc(C9CCC(=O)NC9=O)c8c8cc[nH]c78)C6)CC5)cc4)n3)C2)C1=O]

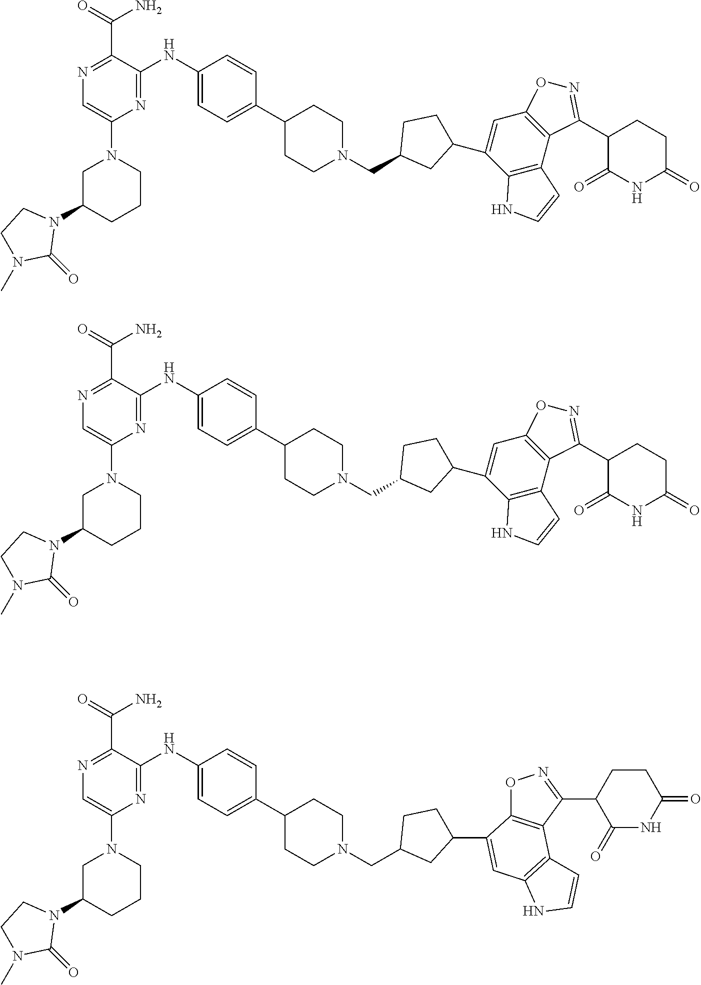 [SMILES: CN1CCN([C@@H]2CCCN(c3cnc(C(N)=O)c(Nc4ccc(C5CCN(C[C@H]6CCC(c7cc8onc(C9CCC(=O)NC9=O)c8c8cc[nH]c78)C6)CC5)cc4)n3)C2)C1=O ; CN1CCN([C@@H]2CCCN(c3cnc(C(N)=O)c(Nc4ccc(C5CCN(C[C@@H]6CCC(c7cc8onc(C9CCC(=O)NC9=O)c8c8cc[nH]c78)C6)CC5)cc4)n3)C2)C1=O ; CN1CCN([C@@H]2CCCN(c3cnc(C(N)=O)c(Nc4ccc(C5CCN(CC6CCC(c7cc8[nH]ccc8c8c(C9CCC(=O)NC9=O)noc78)C6)CC5)cc4)n3)C2)C1=O]

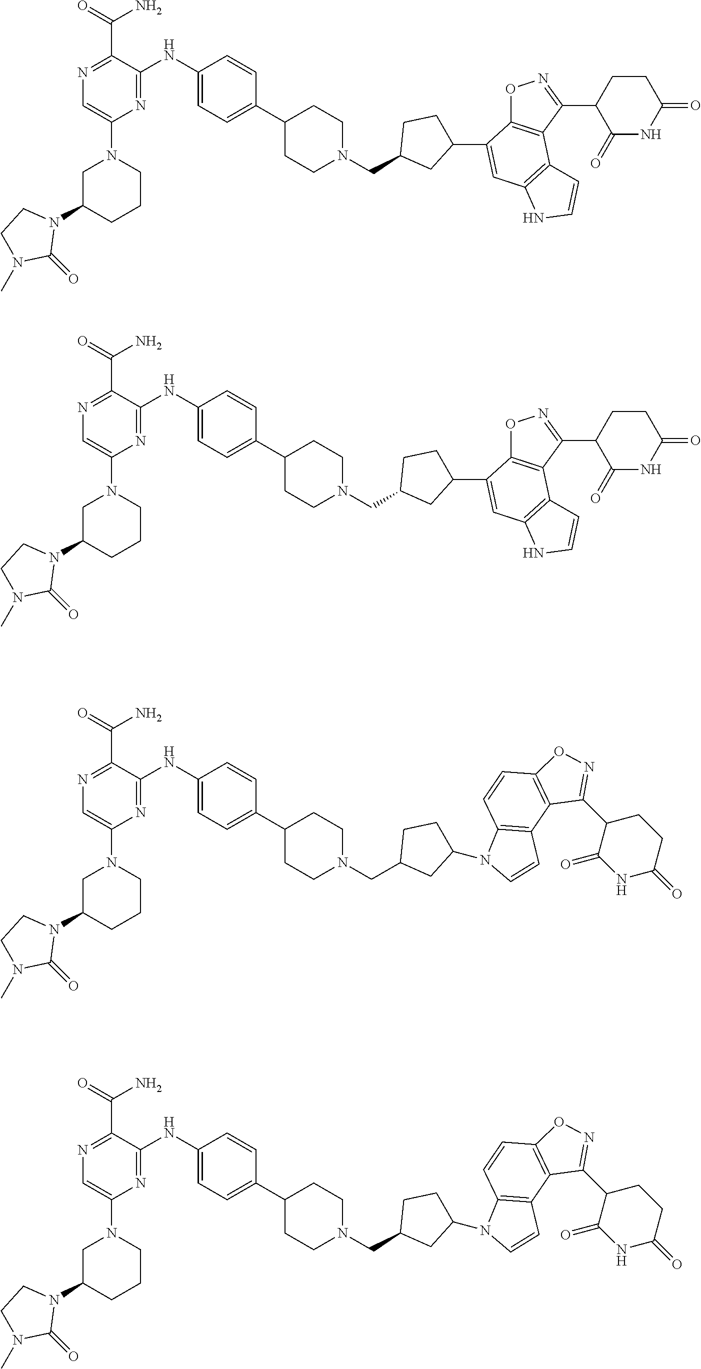 [SMILES: CN1CCN([C@@H]2CCCN(c3cnc(C(N)=O)c(Nc4ccc(C5CCN(C[C@H]6CCC(c7cc8[nH]ccc8c8c(C9CCC(=O)NC9=O)noc78)C6)CC5)cc4)n3)C2)C1=O ; CN1CCN([C@@H]2CCCN(c3cnc(C(N)=O)c(Nc4ccc(C5CCN(C[C@@H]6CCC(c7cc8[nH]ccc8c8c(C9CCC(=O)NC9=O)noc78)C6)CC5)cc4)n3)C2)C1=O ; CN1CCN([C@@H]2CCCN(c3cnc(C(N)=O)c(Nc4ccc(C5CCN(CC6CCC(n7ccc8c9c(C%10CCC(=O)NC%10=O)noc9ccc87)C6)CC5)cc4)n3)C2)C1=O ; CN1CCN([C@@H]2CCCN(c3cnc(C(N)=O)c(Nc4ccc(C5CCN(C[C@H]6CCC(n7ccc8c9c(C%10CCC(=O)NC%10=O)noc9ccc87)C6)CC5)cc4)n3)C2)C1=O]

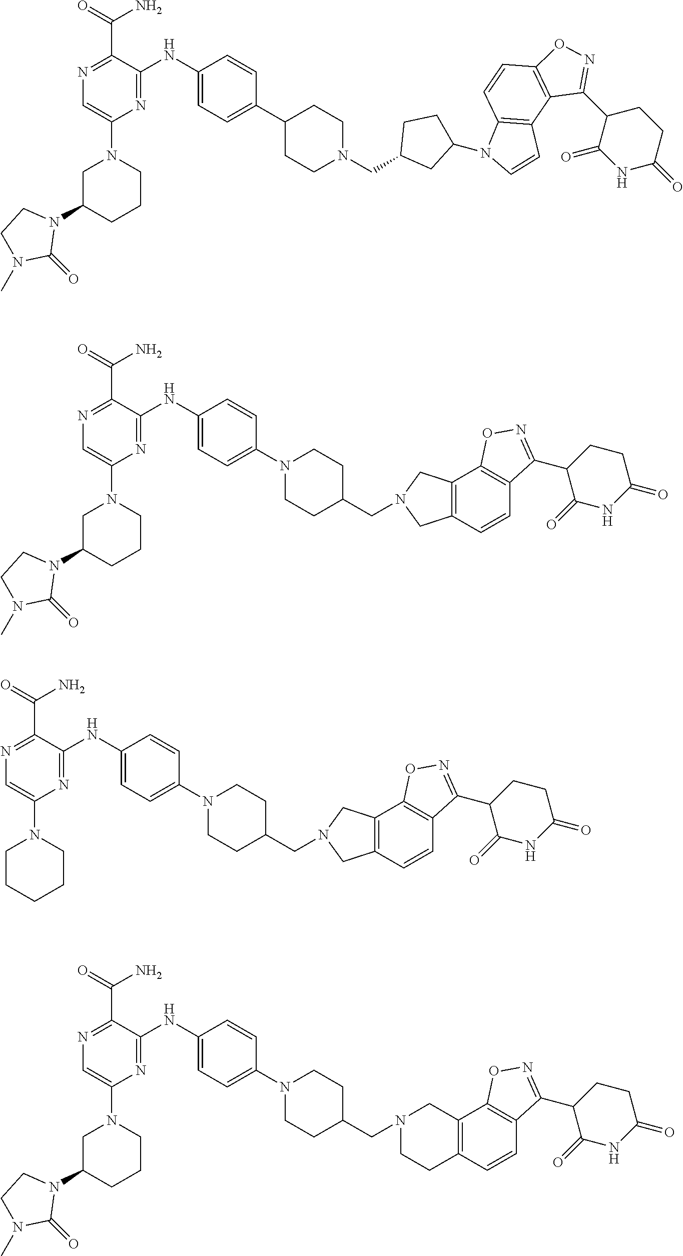 [SMILES: CN1CCN([C@@H]2CCCN(c3cnc(C(N)=O)c(Nc4ccc(C5CCN(C[C@@H]6CCC(n7ccc8c9c(C%10CCC(=O)NC%10=O)noc9ccc87)C6)CC5)cc4)n3)C2)C1=O ; CN1CCN([C@@H]2CCCN(c3cnc(C(N)=O)c(Nc4ccc(N5CCC(CN6Cc7ccc8c(C9CCC(=O)NC9=O)noc8c7C6)CC5)cc4)n3)C2)C1=O ; NC(=O)c1ncc(N2CCCCC2)nc1Nc1ccc(N2CCC(CN3Cc4ccc5c(C6CCC(=O)NC6=O)noc5c4C3)CC2)cc1 ; CN1CCN([C@@H]2CCCN(c3cnc(C(N)=O)c(Nc4ccc(N5CCC(CN6CCc7ccc8c(C9CCC(=O)NC9=O)noc8c7C6)CC5)cc4)n3)C2)C1=O]

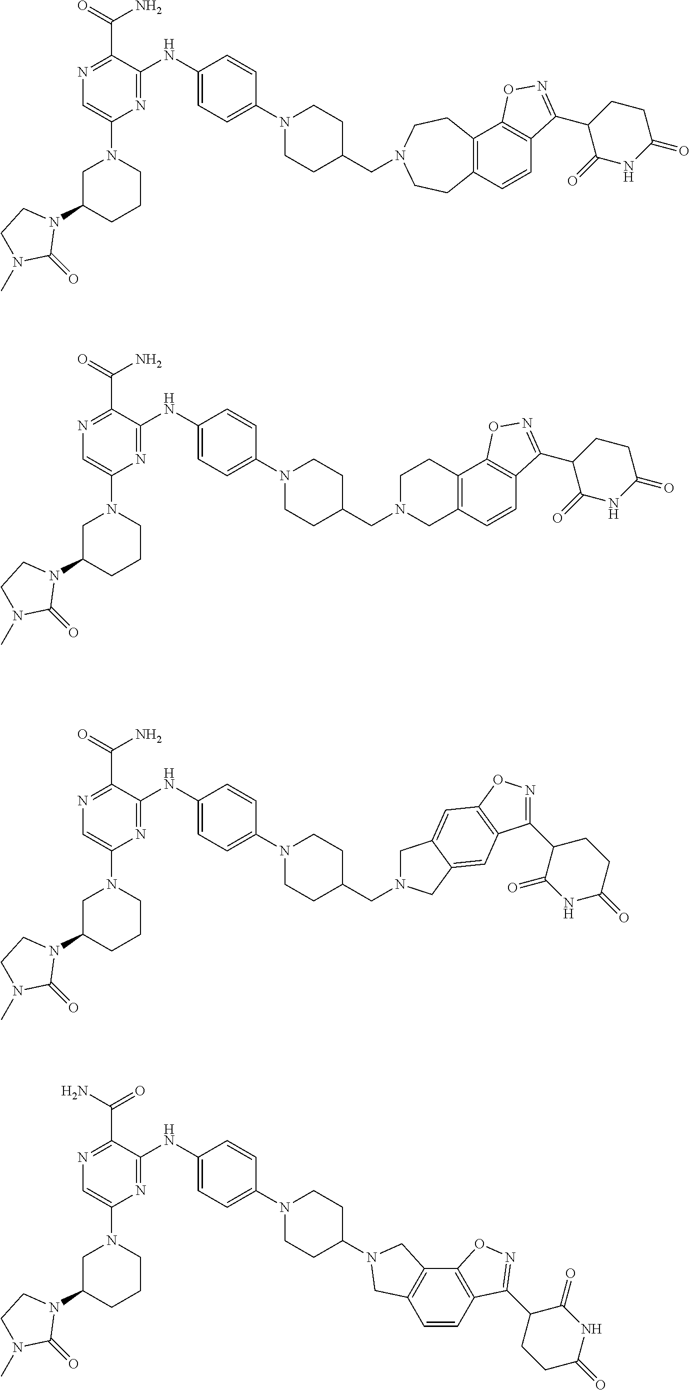 [SMILES: CN1CCN([C@@H]2CCCN(c3cnc(C(N)=O)c(Nc4ccc(N5CCC(CN6CCc7ccc8c(C9CCC(=O)NC9=O)noc8c7CC6)CC5)cc4)n3)C2)C1=O ; CN1CCN([C@@H]2CCCN(c3cnc(C(N)=O)c(Nc4ccc(N5CCC(CN6CCc7c(ccc8c(C9CCC(=O)NC9=O)noc78)C6)CC5)cc4)n3)C2)C1=O ; CN1CCN([C@@H]2CCCN(c3cnc(C(N)=O)c(Nc4ccc(N5CCC(CN6Cc7cc8onc(C9CCC(=O)NC9=O)c8cc7C6)CC5)cc4)n3)C2)C1=O ; CN1CCN([C@@H]2CCCN(c3cnc(C(N)=O)c(Nc4ccc(N5CCC(N6Cc7ccc8c(C9CCC(=O)NC9=O)noc8c7C6)CC5)cc4)n3)C2)C1=O]

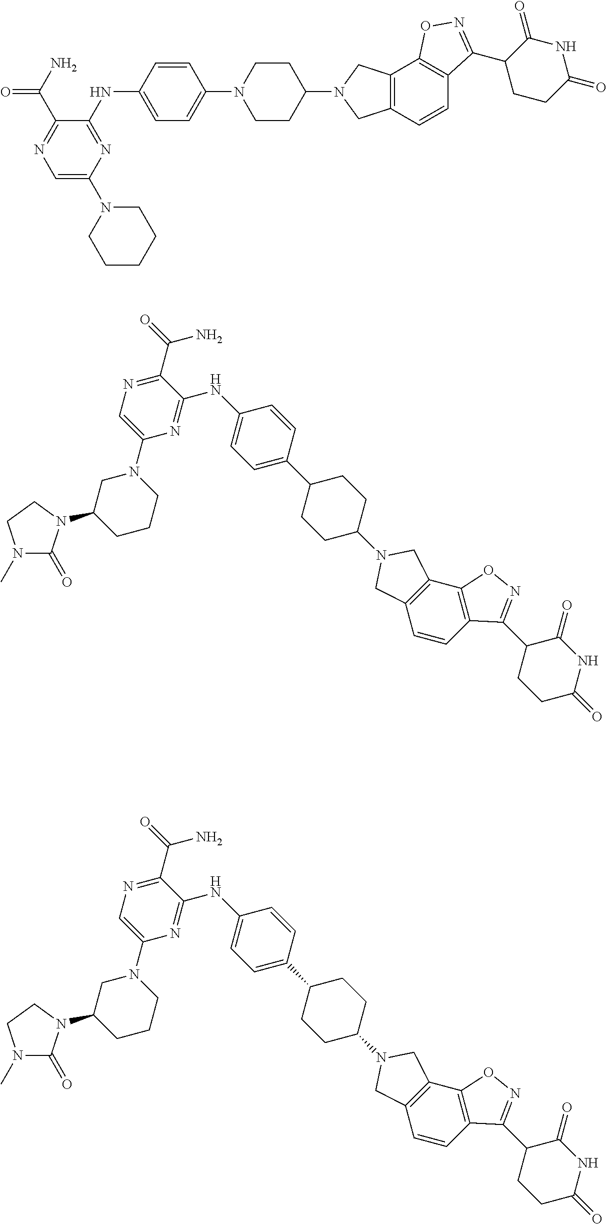 [SMILES: NC(=O)c1ncc(N2CCCCC2)nc1Nc1ccc(N2CCC(N3Cc4ccc5c(C6CCC(=O)NC6=O)noc5c4C3)CC2)cc1 ; CN1CCN([C@@H]2CCCN(c3cnc(C(N)=O)c(Nc4ccc(C5CCC(N6Cc7ccc8c(C9CCC(=O)NC9=O)noc8c7C6)CC5)cc4)n3)C2)C1=O ; CN1CCN([C@@H]2CCCN(c3cnc(C(N)=O)c(Nc4ccc([C@H]5CC[C@@H](N6Cc7ccc8c(C9CCC(=O)NC9=O)noc8c7C6)CC5)cc4)n3)C2)C1=O]

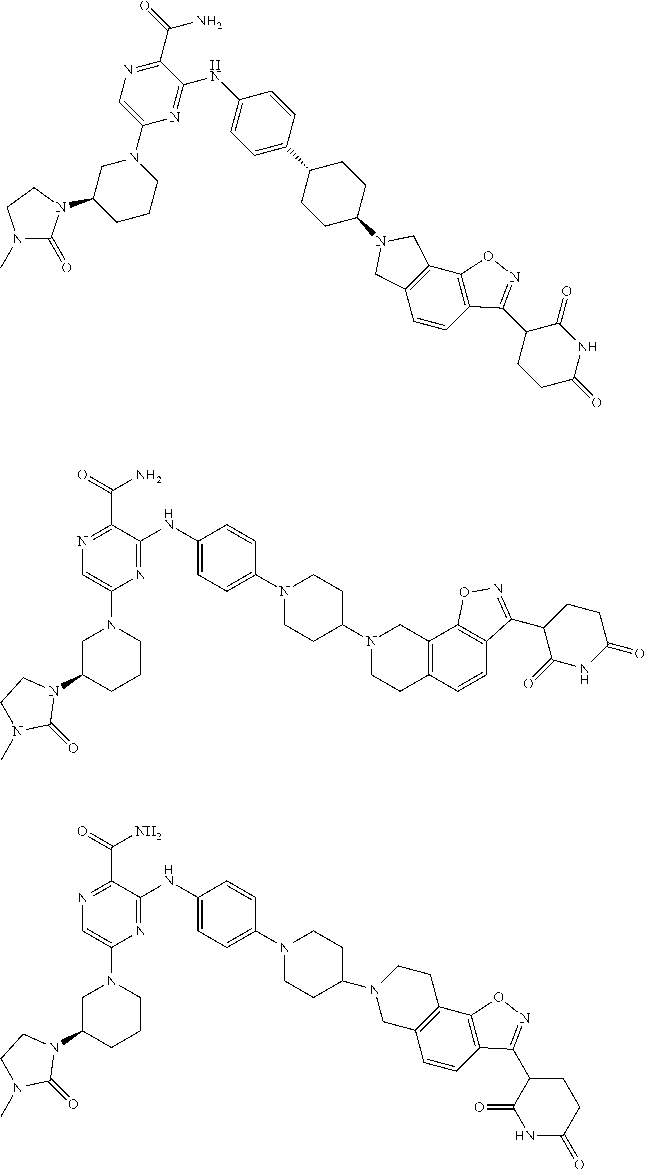 [SMILES: CN1CCN([C@@H]2CCCN(c3cnc(C(N)=O)c(Nc4ccc([C@H]5CC[C@H](N6Cc7ccc8c(C9CCC(=O)NC9=O)noc8c7C6)CC5)cc4)n3)C2)C1=O ; CN1CCN([C@@H]2CCCN(c3cnc(C(N)=O)c(Nc4ccc(N5CCC(N6CCc7ccc8c(C9CCC(=O)NC9=O)noc8c7C6)CC5)cc4)n3)C2)C1=O ; CN1CCN([C@@H]2CCCN(c3cnc(C(N)=O)c(Nc4ccc(N5CCC(N6CCc7c(ccc8c(C9CCC(=O)NC9=O)noc78)C6)CC5)cc4)n3)C2)C1=O]

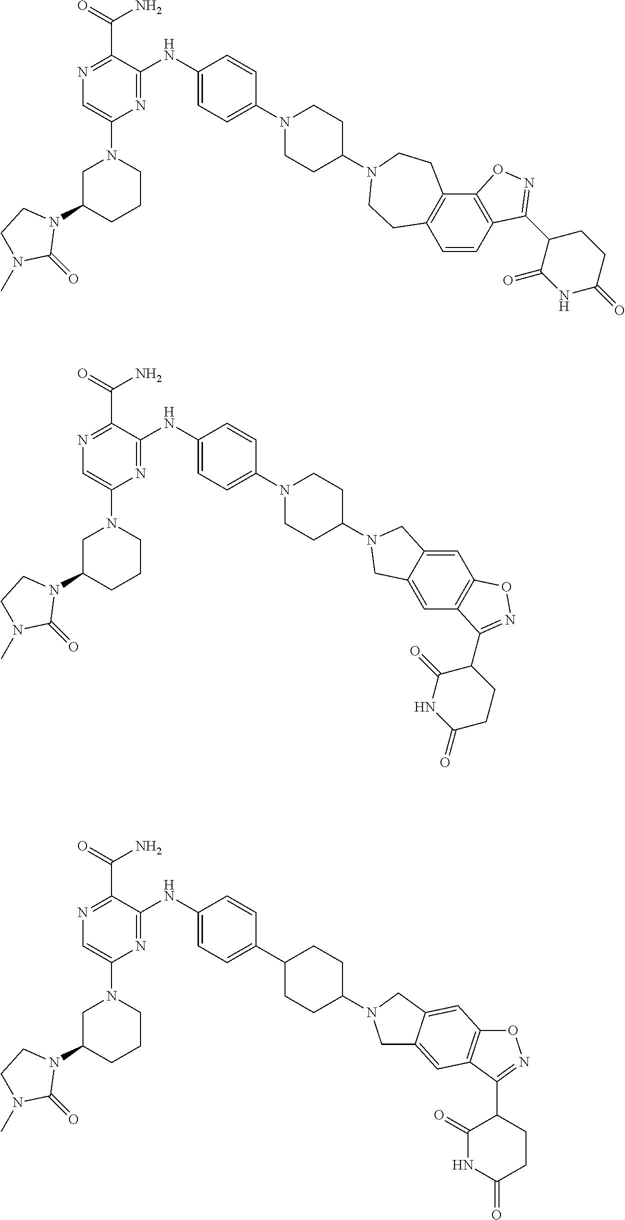 [SMILES: CN1CCN([C@@H]2CCCN(c3cnc(C(N)=O)c(Nc4ccc(N5CCC(N6CCc7ccc8c(C9CCC(=O)NC9=O)noc8c7CC6)CC5)cc4)n3)C2)C1=O ; CN1CCN([C@@H]2CCCN(c3cnc(C(N)=O)c(Nc4ccc(N5CCC(N6Cc7cc8onc(C9CCC(=O)NC9=O)c8cc7C6)CC5)cc4)n3)C2)C1=O ; CN1CCN([C@@H]2CCCN(c3cnc(C(N)=O)c(Nc4ccc(C5CCC(N6Cc7cc8onc(C9CCC(=O)NC9=O)c8cc7C6)CC5)cc4)n3)C2)C1=O]

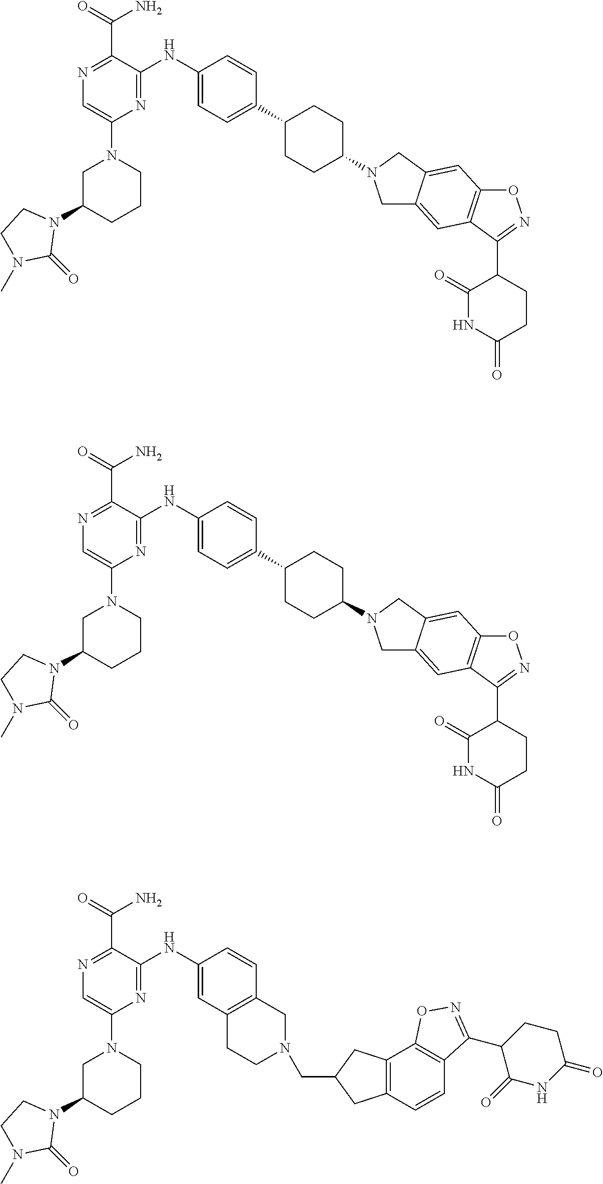 [SMILES: CN1CCN([C@@H]2CCCN(c3cnc(C(N)=O)c(Nc4ccc([C@H]5CC[C@@H](N6Cc7cc8onc(C9CCC(=O)NC9=O)c8cc7C6)CC5)cc4)n3)C2)C1=O ; CN1CCN([C@@H]2CCCN(c3cnc(C(N)=O)c(Nc4ccc([C@H]5CC[C@H](N6Cc7cc8onc(C9CCC(=O)NC9=O)c8cc7C6)CC5)cc4)n3)C2)C1=O ; CN1CCN([C@@H]2CCCN(c3cnc(C(N)=O)c(Nc4ccc5c(c4)CCN(CC4Cc6ccc7c(C8CCC(=O)NC8=O)noc7c6C4)C5)n3)C2)C1=O]

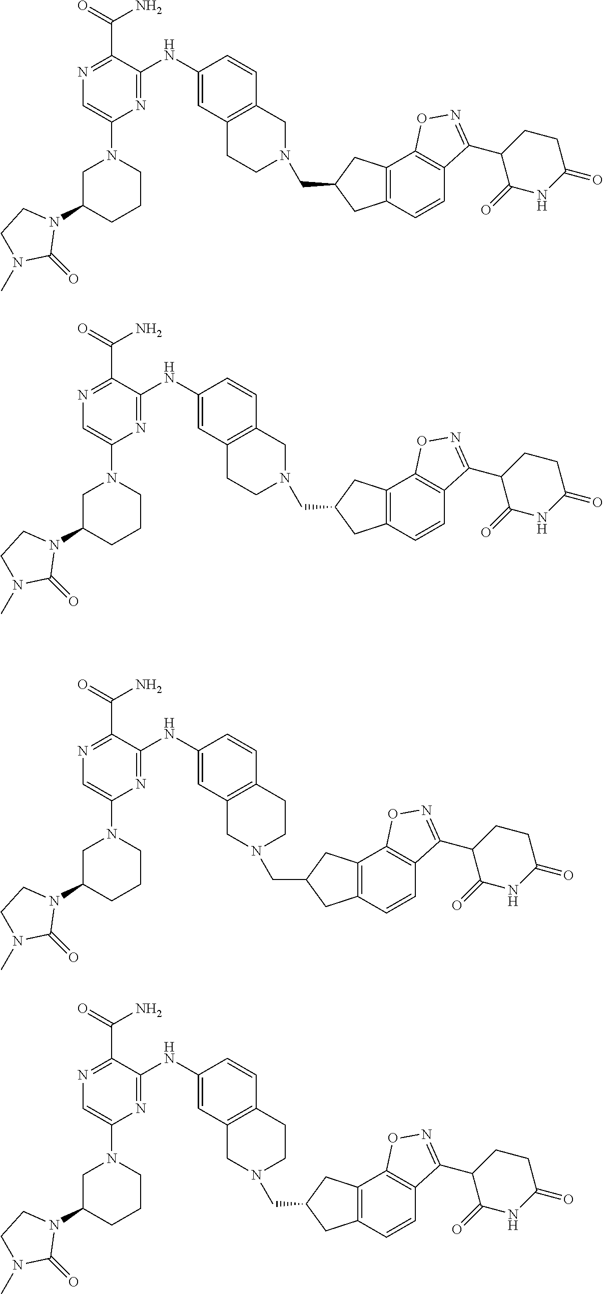 [SMILES: CN1CCN([C@@H]2CCCN(c3cnc(C(N)=O)c(Nc4ccc5c(c4)CCN(C[C@@H]4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CCN(C[C@H]4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CN(CC4Cc6ccc7c(C8CCC(=O)NC8=O)noc7c6C4)CC5)n3)C2)C1=O ; CN1CCN([C@@H]2CCCN(c3cnc(C(N)=O)c(Nc4ccc5c(c4)CN(C[C@H]4Cc6ccc7c(C8CCC(=O)NC8=O)noc7c6C4)CC5)n3)C2)C1=O]

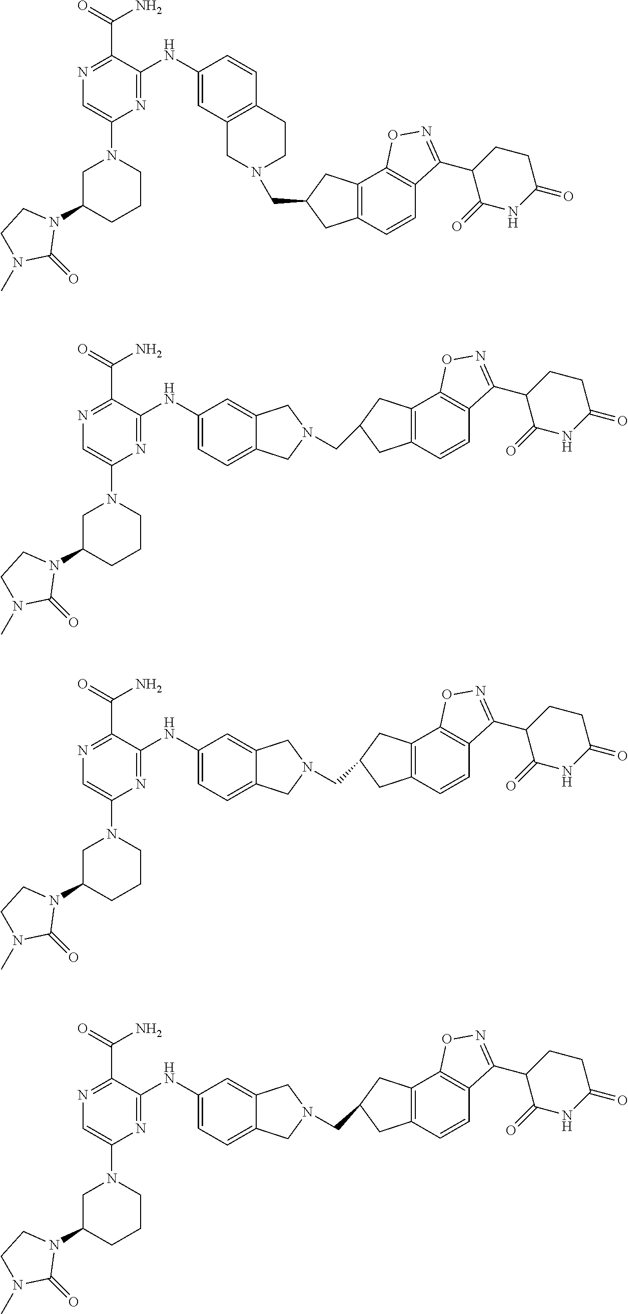 [SMILES: CN1CCN([C@@H]2CCCN(c3cnc(C(N)=O)c(Nc4ccc5c(c4)CN(C[C@@H]4Cc6ccc7c(C8CCC(=O)NC8=O)noc7c6C4)CC5)n3)C2)C1=O ; CN1CCN([C@@H]2CCCN(c3cnc(C(N)=O)c(Nc4ccc5c(c4)CN(CC4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CN(C[C@H]4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CN(C[C@@H]4Cc6ccc7c(C8CCC(=O)NC8=O)noc7c6C4)C5)n3)C2)C1=O]

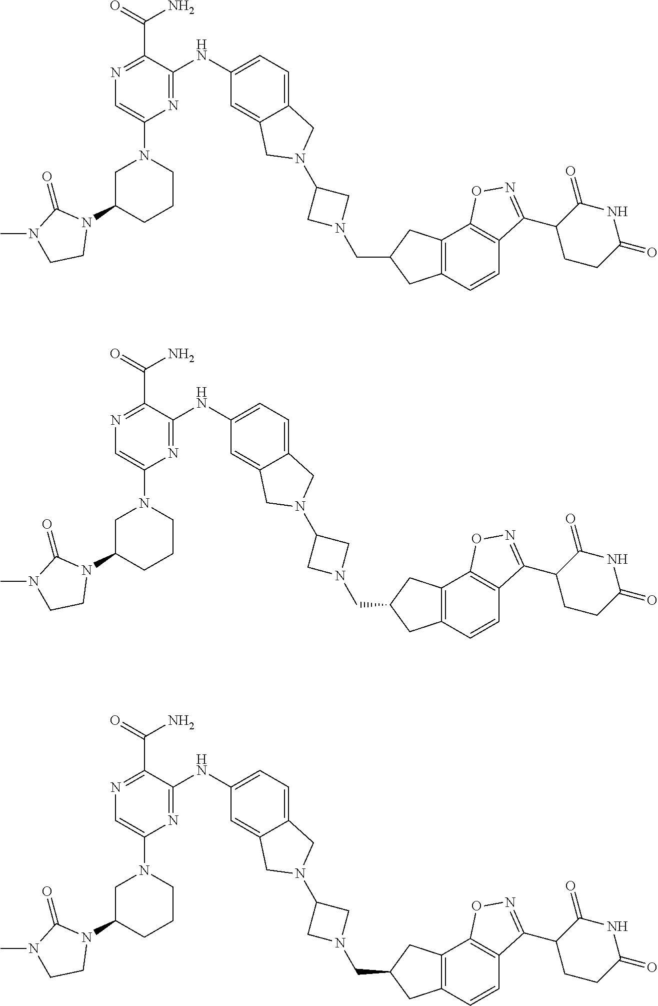 [SMILES: CN1CCN([C@@H]2CCCN(c3cnc(C(N)=O)c(Nc4ccc5c(c4)CN(C4CN(CC6Cc7ccc8c(C9CCC(=O)NC9=O)noc8c7C6)C4)C5)n3)C2)C1=O ; CN1CCN([C@@H]2CCCN(c3cnc(C(N)=O)c(Nc4ccc5c(c4)CN(C4CN(C[C@H]6Cc7ccc8c(C9CCC(=O)NC9=O)noc8c7C6)C4)C5)n3)C2)C1=O ; CN1CCN([C@@H]2CCCN(c3cnc(C(N)=O)c(Nc4ccc5c(c4)CN(C4CN(C[C@@H]6Cc7ccc8c(C9CCC(=O)NC9=O)noc8c7C6)C4)C5)n3)C2)C1=O]

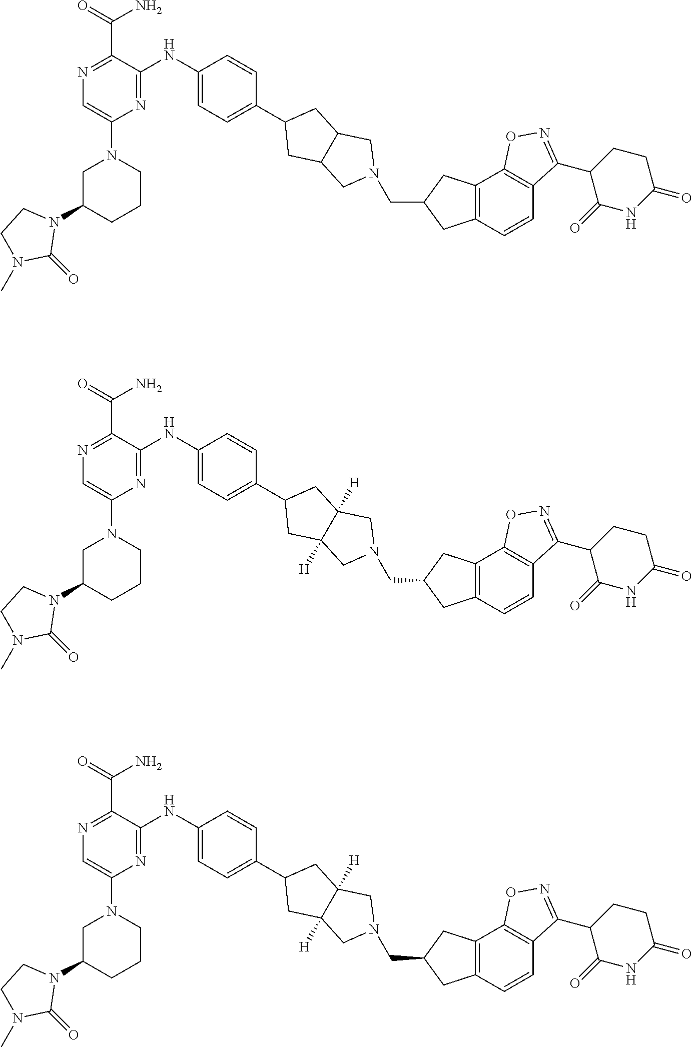 [SMILES: CN1CCN([C@@H]2CCCN(c3cnc(C(N)=O)c(Nc4ccc(C5CC6CN(CC7Cc8ccc9c(C%10CCC(=O)NC%10=O)noc9c8C7)CC6C5)cc4)n3)C2)C1=O ; CN1CCN([C@@H]2CCCN(c3cnc(C(N)=O)c(Nc4ccc(C5C[C@@H]6CN(C[C@H]7Cc8ccc9c(C%10CCC(=O)NC%10=O)noc9c8C7)C[C@@H]6C5)cc4)n3)C2)C1=O ; CN1CCN([C@@H]2CCCN(c3cnc(C(N)=O)c(Nc4ccc(C5C[C@@H]6CN(C[C@@H]7Cc8ccc9c(C%10CCC(=O)NC%10=O)noc9c8C7)C[C@@H]6C5)cc4)n3)C2)C1=O]

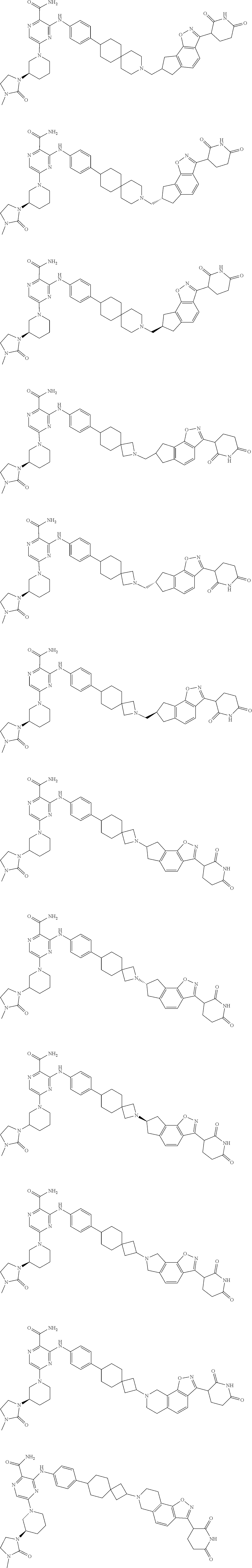

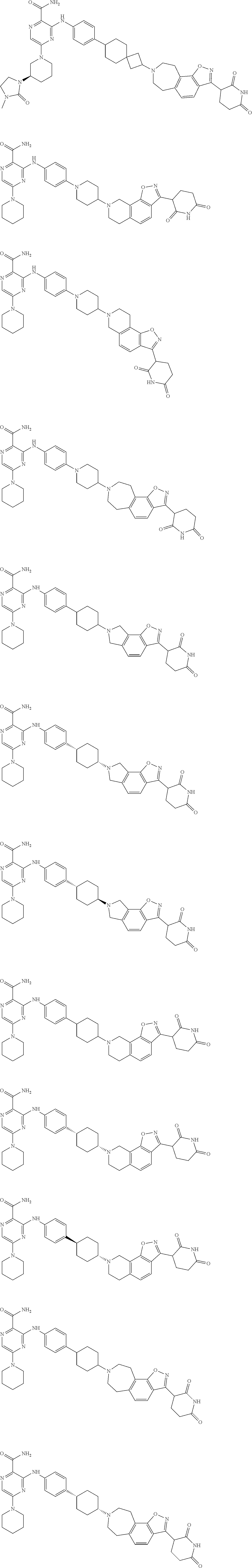

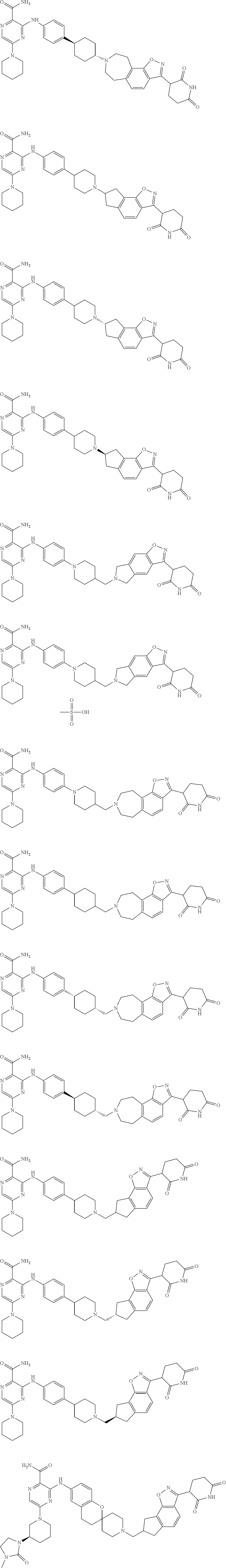

[chemical structure]

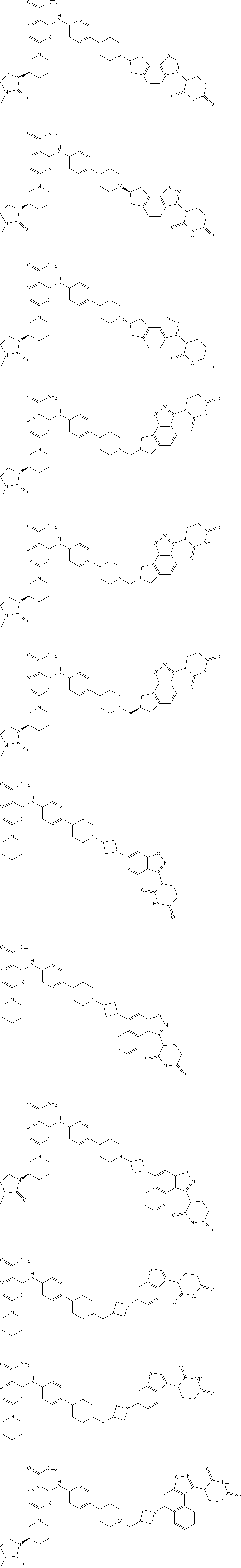

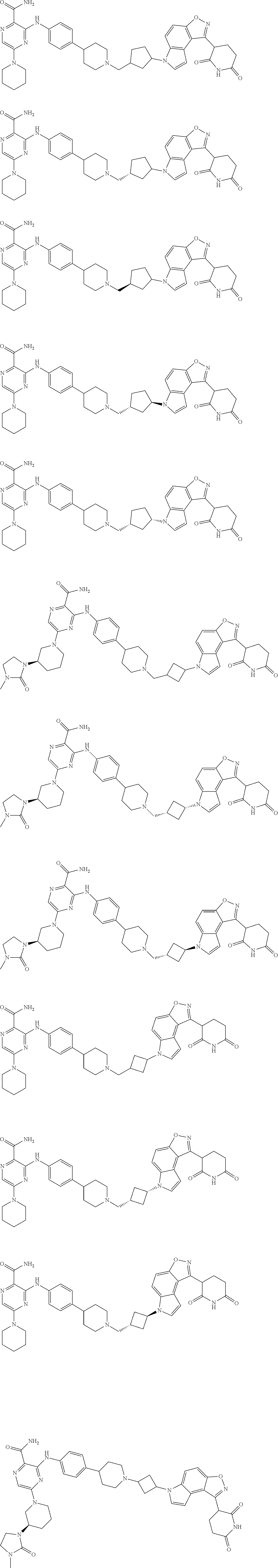

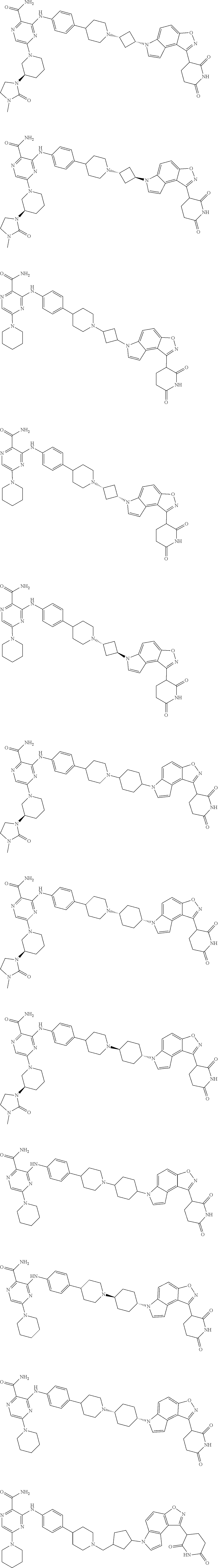

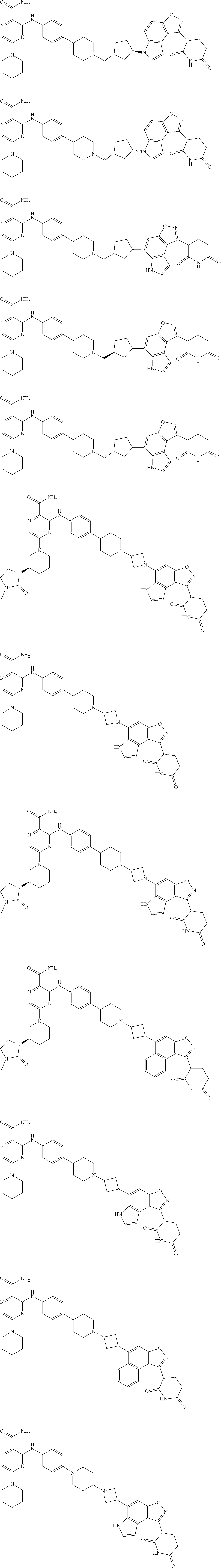

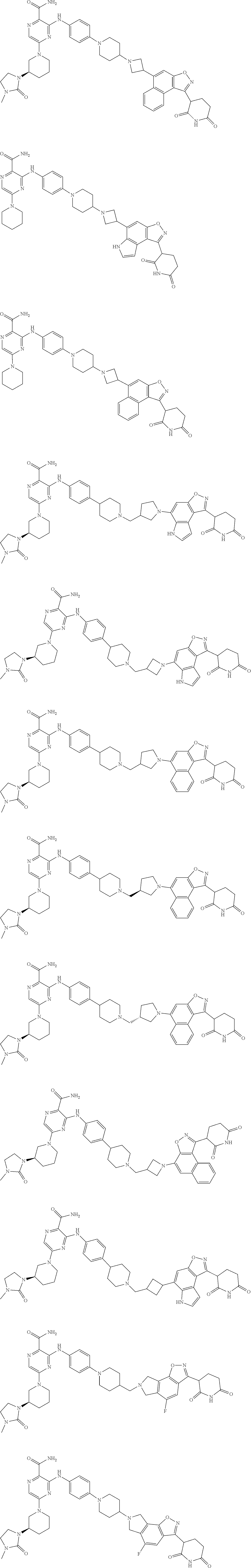

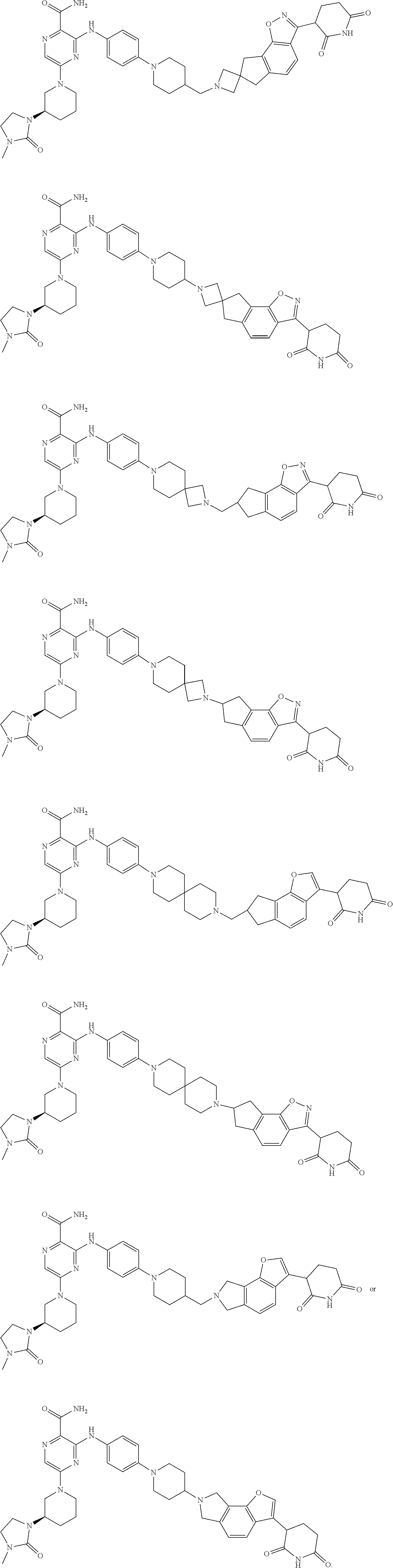

In another aspect, the present application relates to a compound of formula I″, a stereoisomer thereof, or a pharmaceutically acceptable salt thereof,

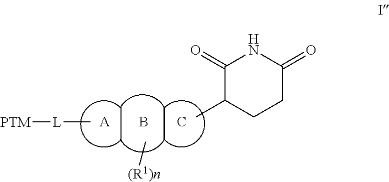

wherein,

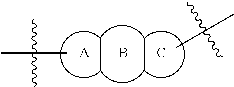

is selected from the group consisting of

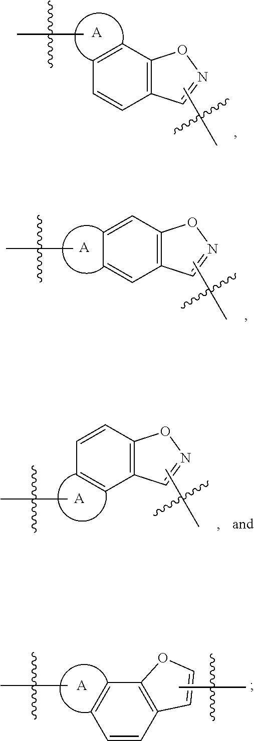

ring A is selected from the group consisting of C₅₋₁₀ cycloalkenyl, 5- to 10-membered heterocycloalkenyl, phenyl, and 5- to 6-membered heteroaryl; or, ring A is selected from the group consisting of C₅₋₈ cycloalkenyl, 5- to 10-membered heterocycloalkenyl, phenyl, and 5- to 6-membered heteroaryl; or, ring A is selected from the group consisting of C₅₋₇ cycloalkenyl, 5- to 10-membered heterocycloalkenyl, phenyl, and 5- to 6-membered heteroaryl; or, ring A is selected from the group consisting of C₅₋₆ cycloalkenyl, 5- to 10-membered heterocycloalkenyl, phenyl, and 5- to 6-membered heteroaryl; or, ring A is selected from the group consisting of C₅₋₁₀ cycloalkenyl and 5- to 10-membered heterocycloalkenyl; or, ring A is selected from the group consisting of C₅₋₈ cycloalkenyl and 5- to 10-membered heterocycloalkenyl; or, ring A is selected from the group consisting of C₅₋₆ cycloalkenyl and 5- to 9-membered heterocycloalkenyl;
PTM is selected from the group consisting of drugs and derivatives thereof that bind to the target protein;
L is selected from a connecting group;
R¹ and n are as defined herein.

In another aspect, the present application relates to a compound of formula I″-1, a moiety, a stereoisomer thereof, a derivative (such as a Protac molecule), or a pharmaceutically acceptable salt thereof,

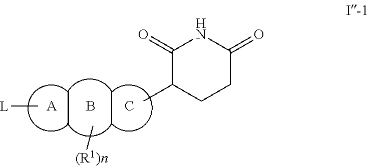

wherein,

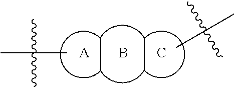

is selected from the group consisting of

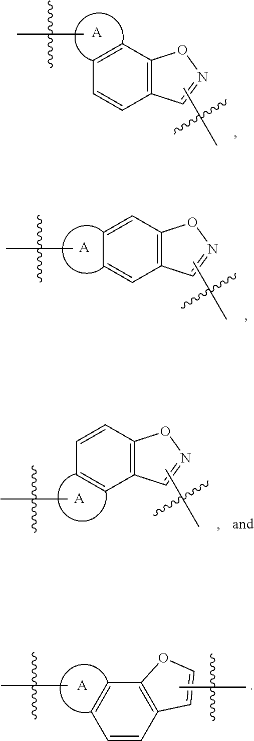

ring A is selected from the group consisting of C₅₋₁₀ cycloalkenyl, 5- to 10-membered heterocycloalkenyl, phenyl, and 5- to 6-membered heteroaryl; or, ring A is selected from the group consisting of C₅₋₈ cycloalkenyl, 5- to 10-membered heterocycloalkenyl, phenyl, and 5- to 6-membered heteroaryl; or, ring A is selected from the group consisting of C₅₋₇ cycloalkenyl, 5- to 10-membered heterocycloalkenyl, phenyl, and 5- to 6-membered heteroaryl; or, ring A is selected from the group consisting of C₅₋₆ cycloalkenyl, 5- to 10-membered heterocycloalkenyl, phenyl, and 5- to 6-membered heteroaryl; or, ring A is selected from the group consisting of C₅₋₁₀ cycloalkenyl and 5- to 10-membered heterocycloalkenyl; or, ring A is selected from the group consisting of C₅₋₈ cycloalkenyl and 5- to 10-membered heterocycloalkenyl; or, ring A is selected from the group consisting of C₅₋₆ cycloalkenyl and 5- to 9-membered heterocycloalkenyl;
L is selected from a connecting group;
R¹ and n are as defined herein.

In some embodiments of the present application, the compound of formula I″-1, the moiety, the stereoisomer thereof, the derivative (such as a Protac molecule), or the pharmaceutically acceptable salt thereof is selected from the group consisting of a compound of formula I″, a stereoisomer thereof, and a pharmaceutically acceptable salt thereof.

In some embodiments, ring A is selected from the group consisting of C₅₋₈ cycloalkenyl (or C₅₋₇ cycloalkenyl), 5- to 9-membered heterocycloalkenyl, phenyl, pyrrolyl, pyrazolyl, furanyl, and oxazolyl.

In some embodiments, ring A is selected from the group consisting of C₅₋₆ cycloalkenyl, 5- to 9-membered heterocycloalkenyl, phenyl, pyrrolyl, pyrazolyl, furanyl, and oxazolyl.

In some embodiments, ring A is selected from the group consisting of C₅ cycloalkenyl, C₆ cycloalkenyl, 5-, 6-, 7-, 8-, or 9-membered heterocycloalkenyl, phenyl, pyrrolyl, pyrazolyl, furanyl, and oxazolyl.

In some embodiments, ring A is selected from the group consisting of cyclopentenyl, bicyclohexenyl, dihydropyrrolyl, tetrahydropyridinyl, tetrahydroazepinyl, azaspirooctenyl, dihydrooxazinyl, azaspirononenyl, phenyl, pyrrolyl, pyrazolyl, furanyl, and oxazolyl.

In some specific embodiments, ring A is selected from the group consisting of C₅₋₈ cycloalkenyl and 5- to 9-membered heterocycloalkenyl. In some specific embodiments, ring A is selected from the group consisting of C₅₋₇ cycloalkenyl and 5- to 9-membered heterocycloalkenyl. In some specific embodiments, ring A is selected from the group consisting of C₅₋₆ cycloalkenyl and 5- to 9-membered heterocycloalkenyl. In some specific embodiments, ring A is selected from 5- to 8-membered heterocycloalkenyl. In some specific embodiments, ring A is selected from the group consisting of 6- to 8-membered heterocycloalkenyl. In some specific embodiments, ring A is selected from 5-membered heterocycloalkenyl. In some specific embodiments, ring A is selected from 6-membered heterocycloalkenyl. In some specific embodiments, ring A is selected from 7-membered heterocycloalkenyl. In some specific embodiments, ring A is selected from 8-membered heterocycloalkenyl. In some specific embodiments, ring A is selected from 9-membered heterocycloalkenyl.

In some specific embodiments, ring A is selected from the group consisting of cyclopentenyl, bicyclohexenyl, dihydropyrrolyl, tetrahydropyridinyl, dihydrooxazinyl, tetrahydroazepinyl, azaspirooctenyl, and azaspirononenyl.

In some specific embodiments, ring A is selected from the group consisting of cyclopentenyl and bicyclohexenyl.

In some embodiments, ring A is selected from the group consisting of dihydropyrrolyl, dihydrooxazinyl, tetrahydropyridinyl, tetrahydroazepinyl, azaspirooctenyl, and azaspirononenyl.

In some embodiments, ring A is selected from the group consisting of phenyl, pyrrolyl, pyrazolyl, furanyl, and oxazolyl.

In some other embodiments, ring A is selected from the group consisting of C₅₋₇ cycloalkenyl, 5- to 8-membered heterocycloalkenyl, phenyl, and pyrrolyl.

In some other embodiments, ring A is selected from the group consisting of C₅₋₆ cycloalkenyl, 5- to 8-membered heterocycloalkenyl, phenyl, and pyrrolyl.

In some other embodiments, ring A is selected from the group consisting of C₅ cycloalkenyl, 5-, 6-, 7-, or 8-membered heterocycloalkenyl, phenyl, and pyrrolyl.

In some other embodiments, ring A is selected from the group consisting of cyclopentenyl, dihydropyrrolyl, tetrahydropyridinyl, tetrahydroazepinyl, azaspirooctene, phenyl, and pyrrolyl.

In some other embodiments, ring A is selected from the group consisting of C₅₋₆ cycloalkenyl and 5- to 8-membered heterocycloalkenyl.

In some other embodiments, ring A is selected from the group consisting of cyclopentenyl, dihydropyrrolyl, tetrahydropyridinyl, tetrahydroazepinyl, and azaspirooctene.

In some other embodiments, ring A is selected from cyclopentenyl. In some embodiments, ring A is selected from the group consisting of dihydropyrrolyl, tetrahydropyridinyl, tetrahydroazepinyl, and azaspirooctene.

In some other embodiments, ring A is selected from the group consisting of phenyl and pyrrolyl.

In some embodiments, the structural moiety

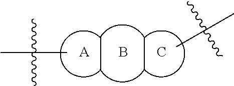

is selected from the group consisting of

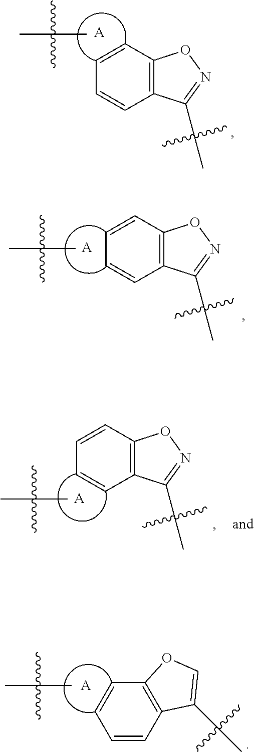

In some embodiments, the structural moiety

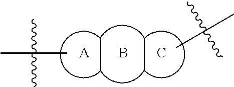

is selected from the group consisting of

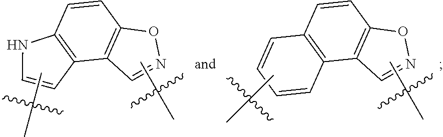

in some embodiments, the structural moiety

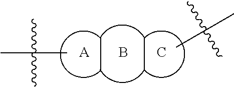

is selected from the group consisting of

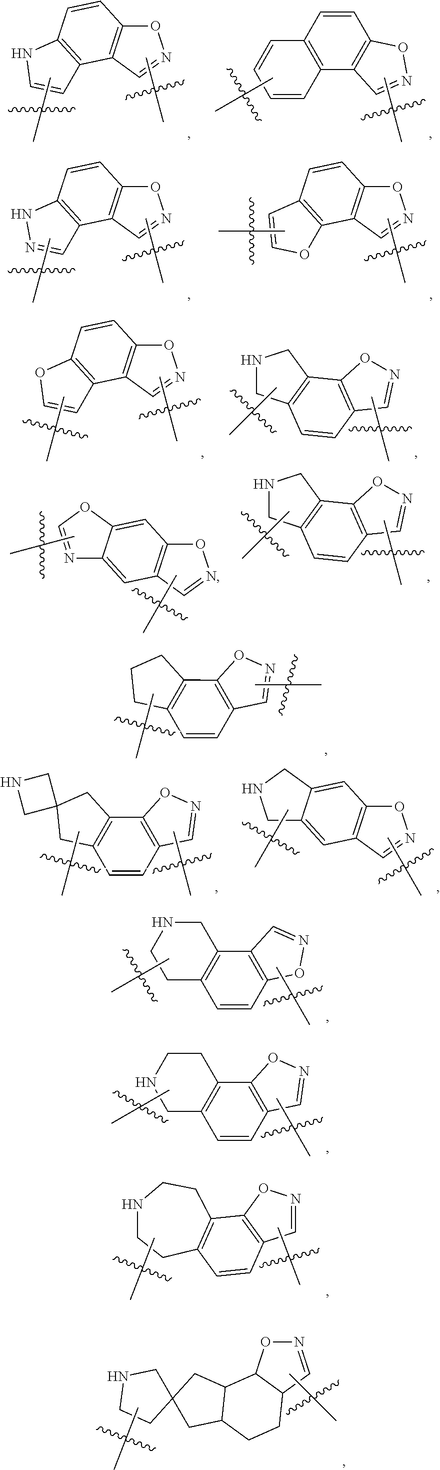

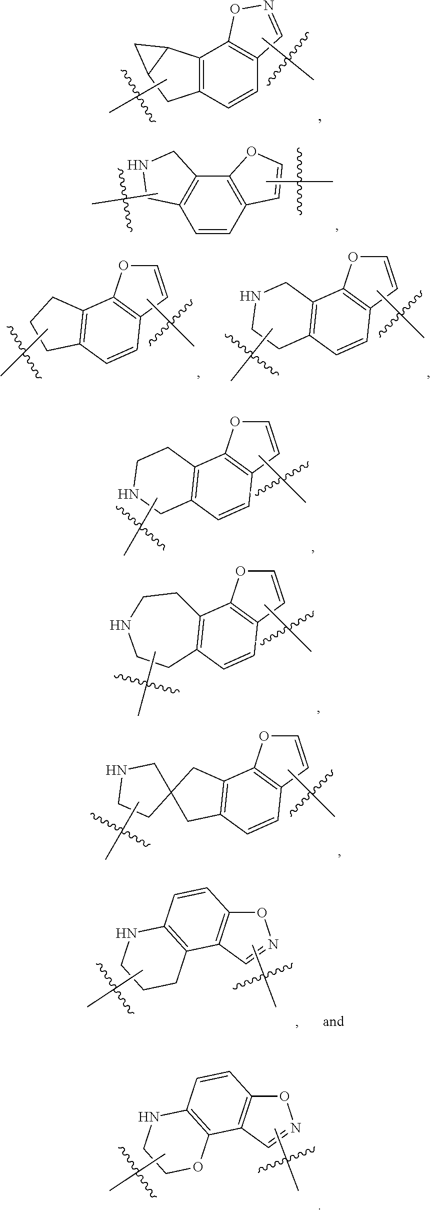

In some other embodiments, the structural moiety

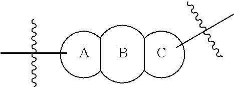

is selected from the group consisting of

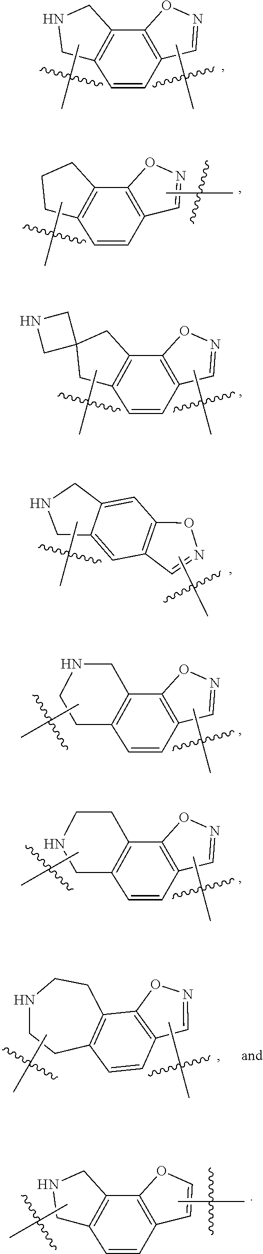

In some embodiments, the strutural moiety

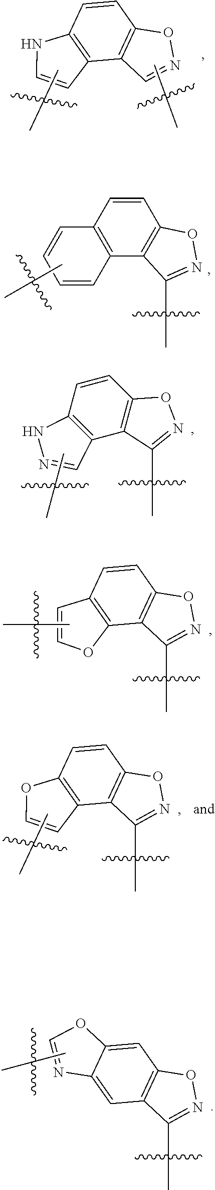

In some

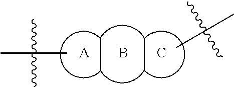

embodiments, the structural moiety

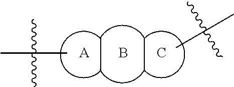

is selected from the group consisting of

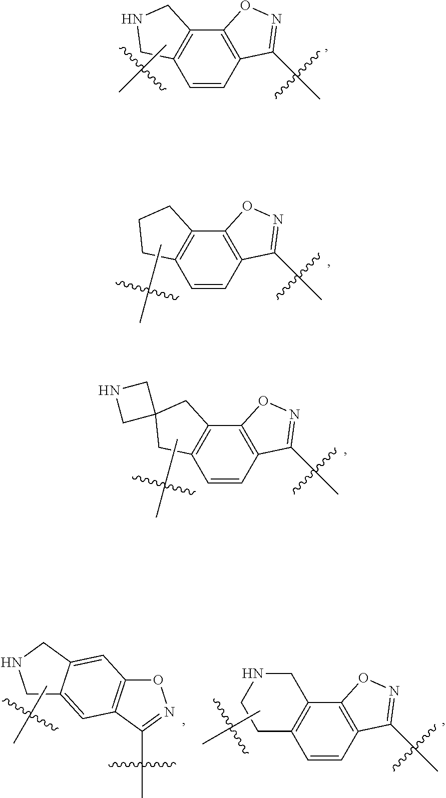

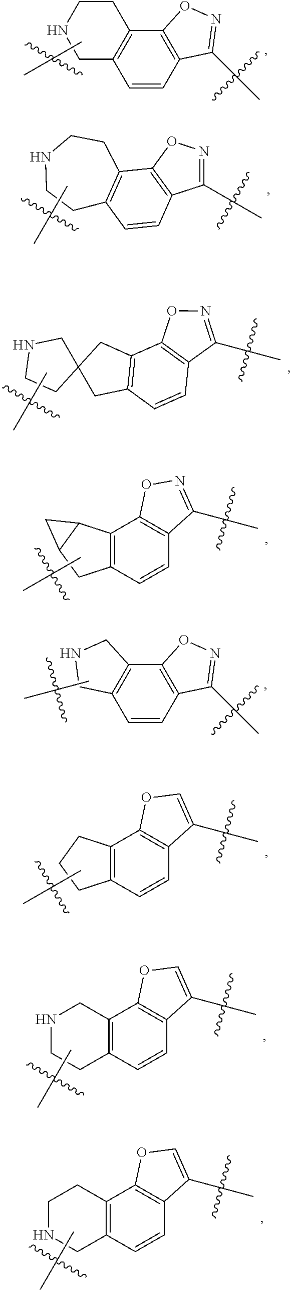

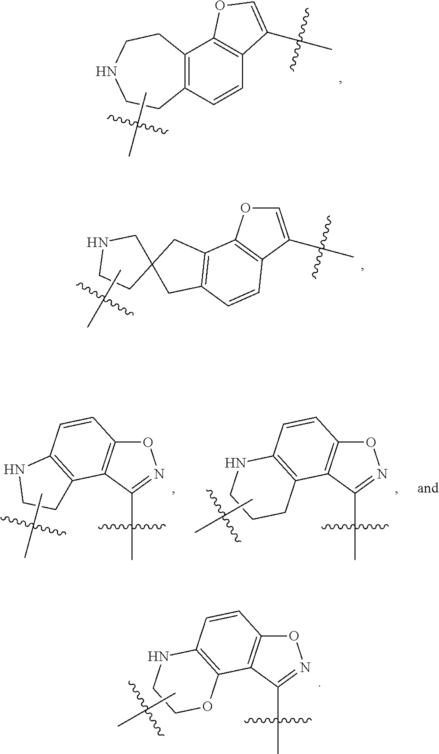

In some embodiments, the structural moiety

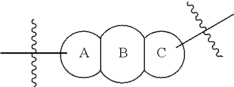

is selected from the group consisting of

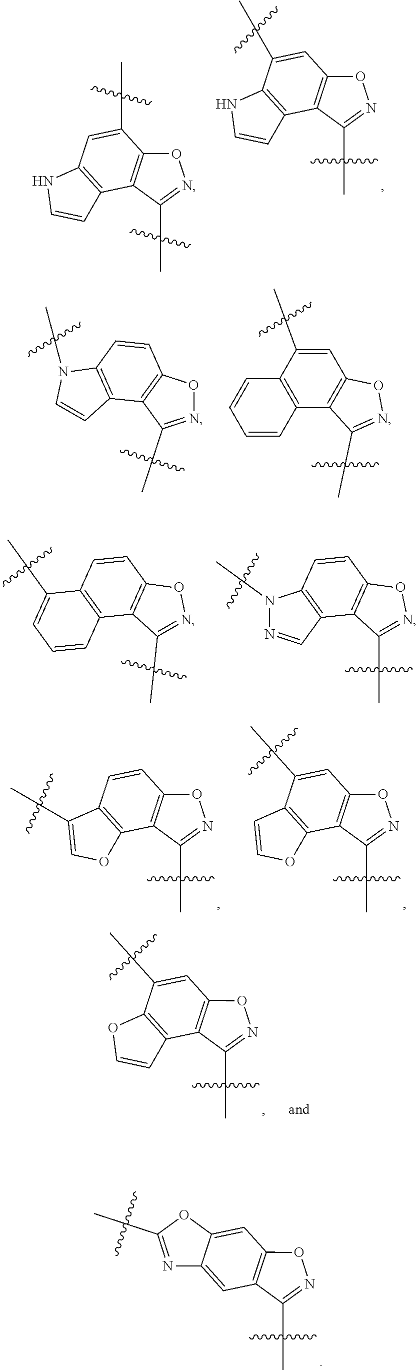

In some embodiments, the structural moiety

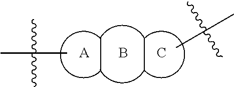

is selected from the group consisting of

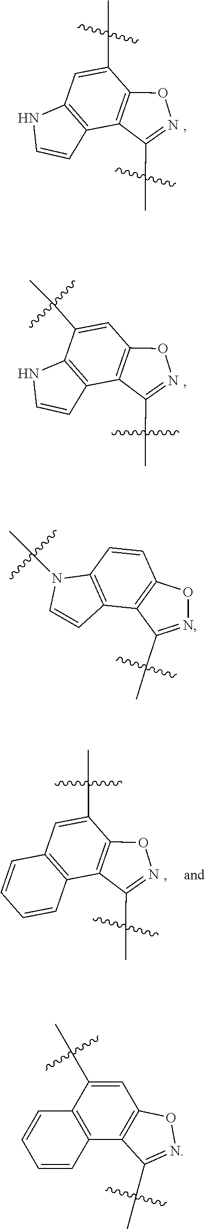 [SMILES: *c1cc2[nH]ccc2c2c(*)noc12 ; *c1cc2onc(*)c2c2cc[nH]c12 ; *c1noc2ccc3c(ccn3*)c12 ; *c1cc2ccccc2c2c(*)noc12 ; *c1cc2onc(*)c2c2ccccc12]

In some embodiments, the structural moiety

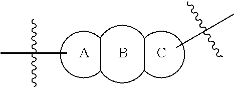

is selected from the group consisting of

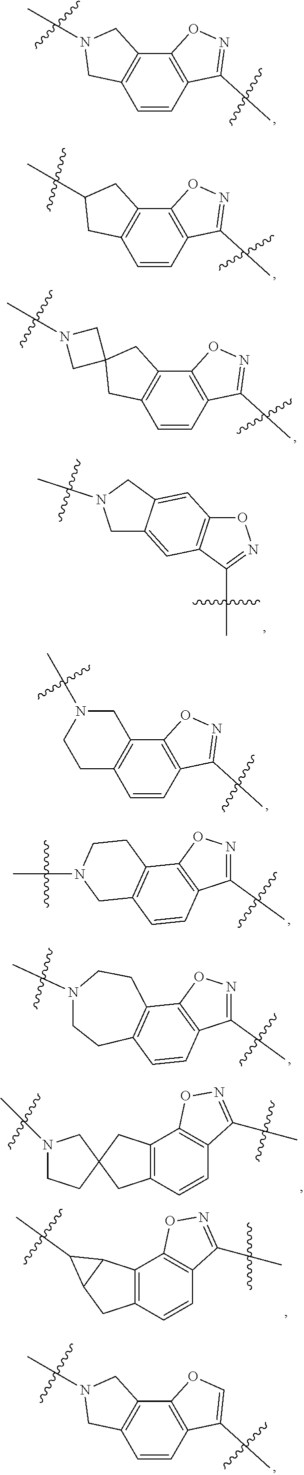

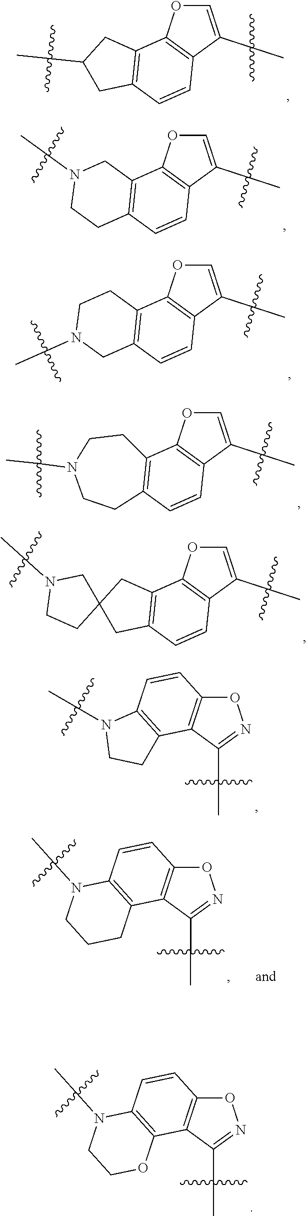

In some other embodiments, the structural moiety

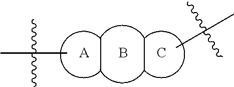

is selected from the group consisting of

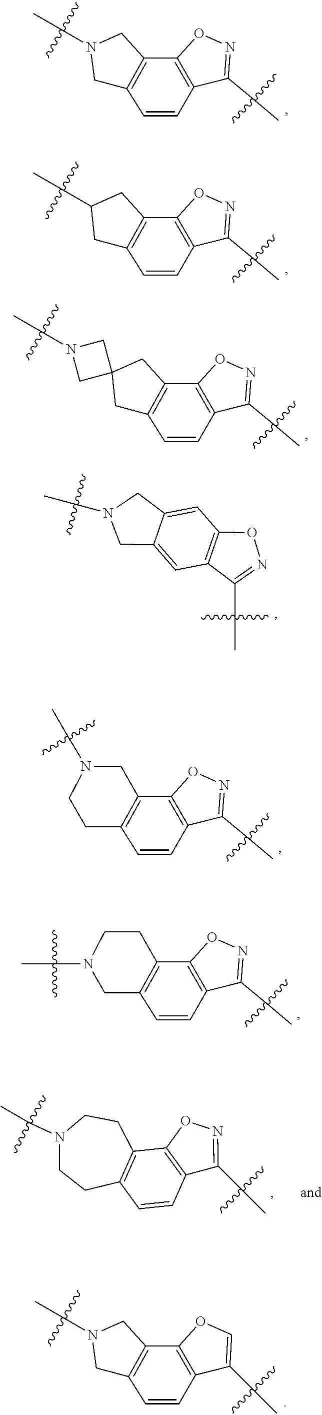

In some embodiments, the structural moiety

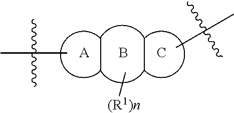

is selected from the group consisting of

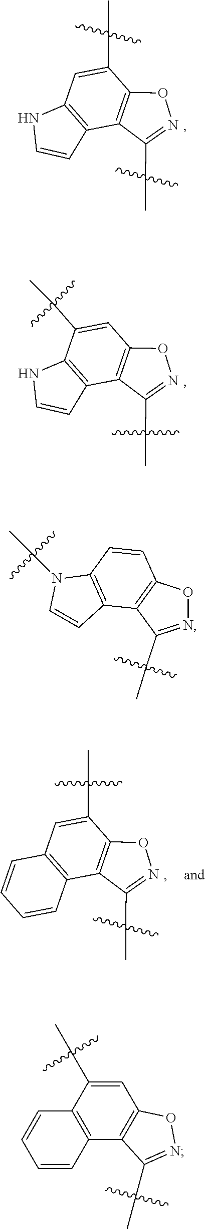 [SMILES: *c1cc2[nH]ccc2c2c(*)noc12 ; *c1cc2onc(*)c2c2cc[nH]c12 ; *c1noc2ccc3c(ccn3*)c12 ; *c1cc2ccccc2c2c(*)noc12 ; *c1cc2onc(*)c2c2ccccc12]

in some embodiments, the structural moiety

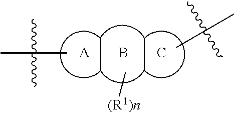

is selected from the group consisting of

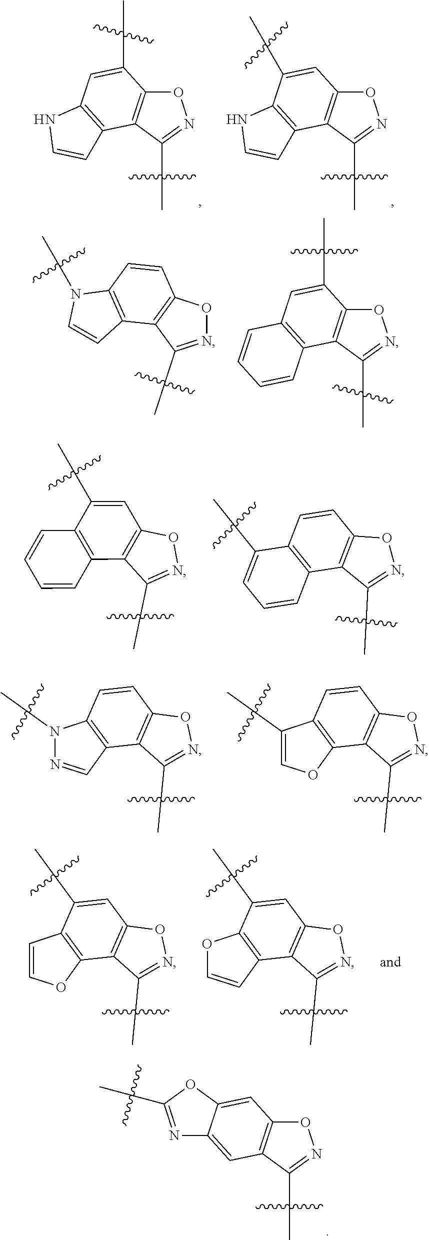

In some embodiments, the structural moiety

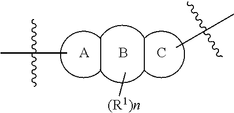

is selected from the group consisting of

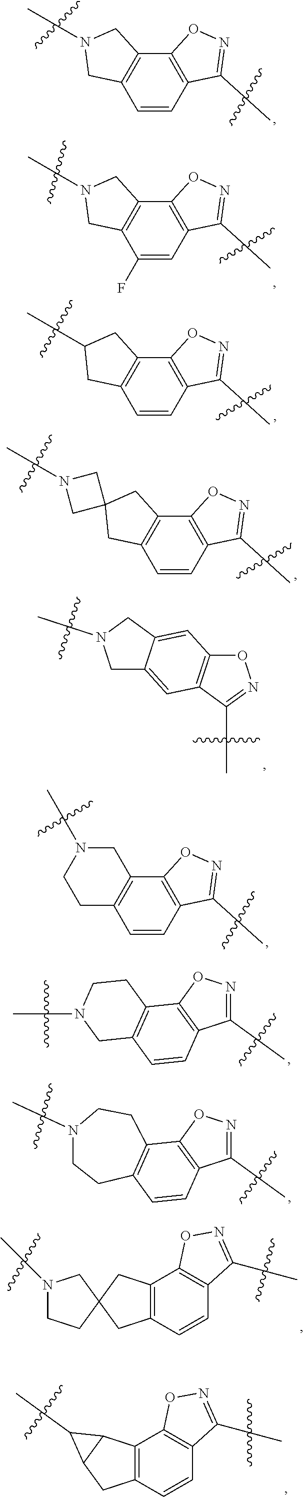

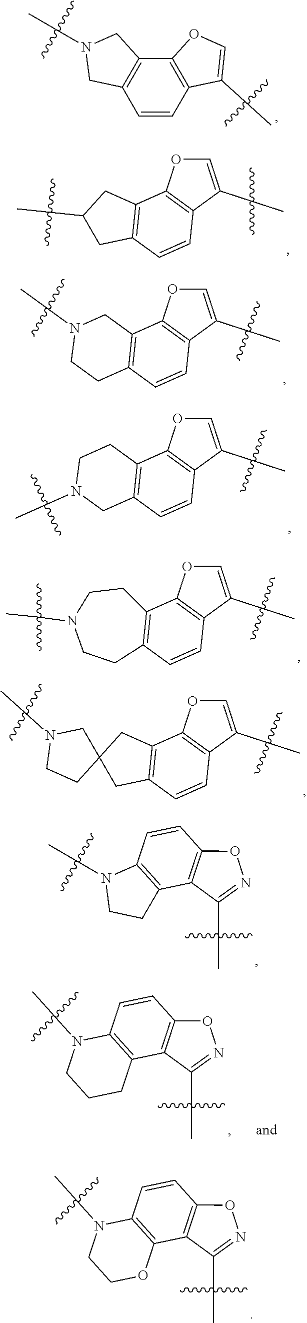

In some embodiments, the structural moiety

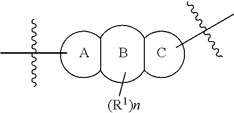

is selected from the group consisting of

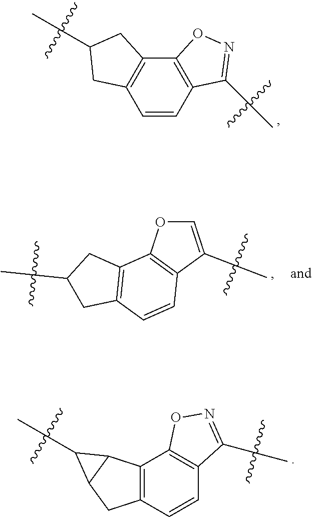 [SMILES: *c1noc2c3c(ccc12)CC(*)C3 ; *c1coc2c3c(ccc12)CC(*)C3 ; *c1noc2c3c(ccc12)CC1C(*)C31]

In some embodiments, the structural moiety

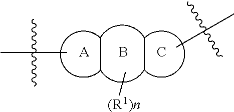

is selected from the group consisting of

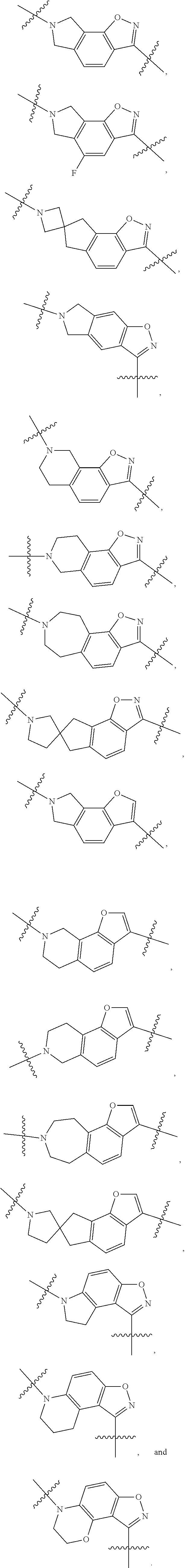

In some other embodiments, the structural moiety

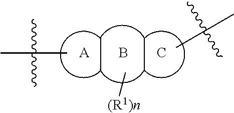

is selected from the group consisting of

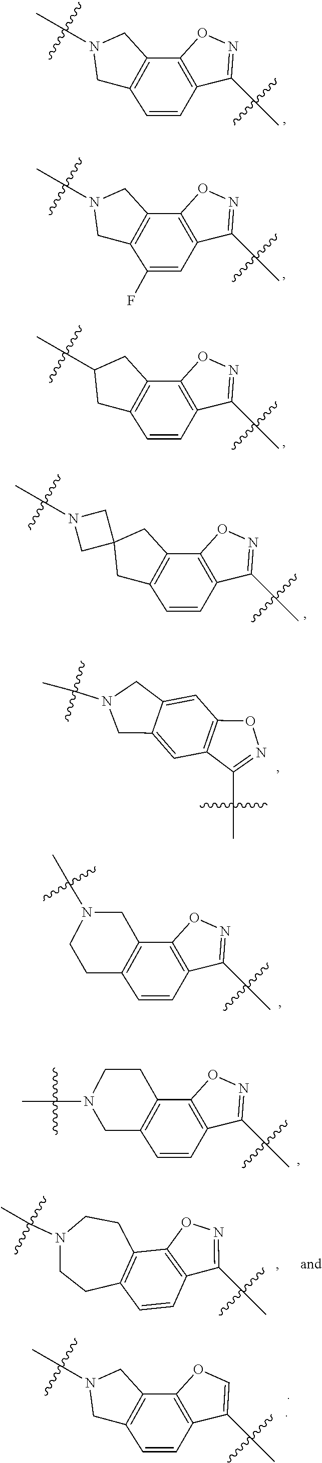

In some embodiments, L is selected from the group consisting of -Cy¹-LNK-Cy²-LNK-, -Cy¹-LNK-Cy²-, and -Cy¹-Cy²-LNK-; in some embodiments, L is selected from -Cy¹-LNK-Cy²-; in some embodiments, Cy¹, LNK, Cy², -Cy¹-LNK-, -LNK-Cy²-, -Cy¹-Cy²-, or -Cy¹-LNK-Cy²- is as described herein; further, in some embodiments, the moiety

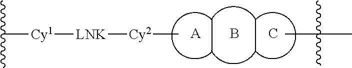

is selected from the group consisting of

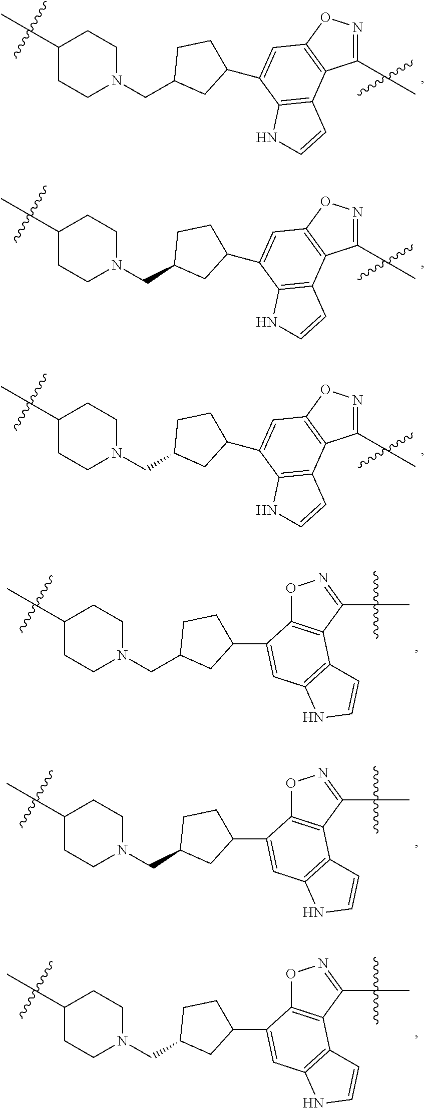 [SMILES: *c1noc2cc(C3CCC(CN4CCC(*)CC4)C3)c3[nH]ccc3c12 ; *c1noc2cc(C3CC[C@H](CN4CCC(*)CC4)C3)c3[nH]ccc3c12 ; *c1noc2cc(C3CC[C@@H](CN4CCC(*)CC4)C3)c3[nH]ccc3c12 ; *c1noc2c(C3CCC(CN4CCC(*)CC4)C3)cc3[nH]ccc3c12 ; *c1noc2c(C3CC[C@H](CN4CCC(*)CC4)C3)cc3[nH]ccc3c12 ; *c1noc2c(C3CC[C@@H](CN4CCC(*)CC4)C3)cc3[nH]ccc3c12]

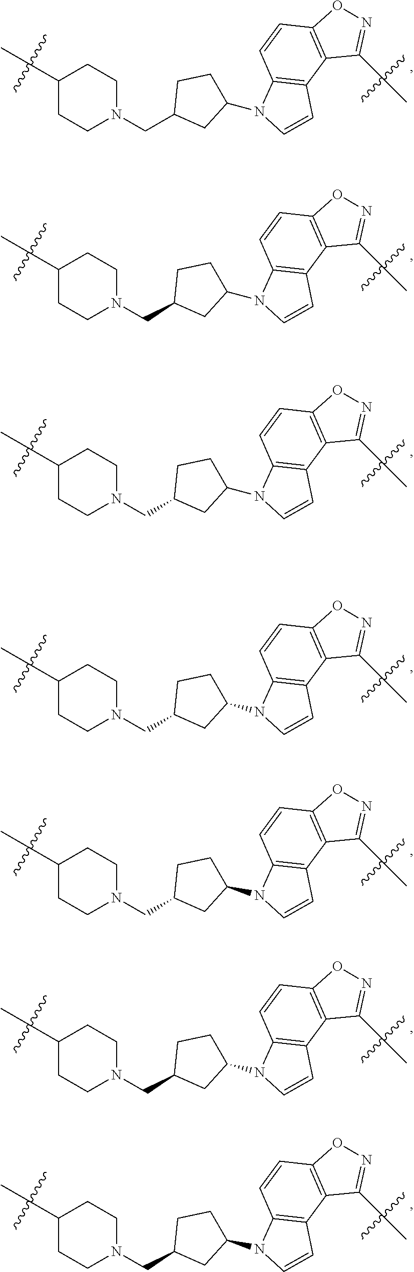

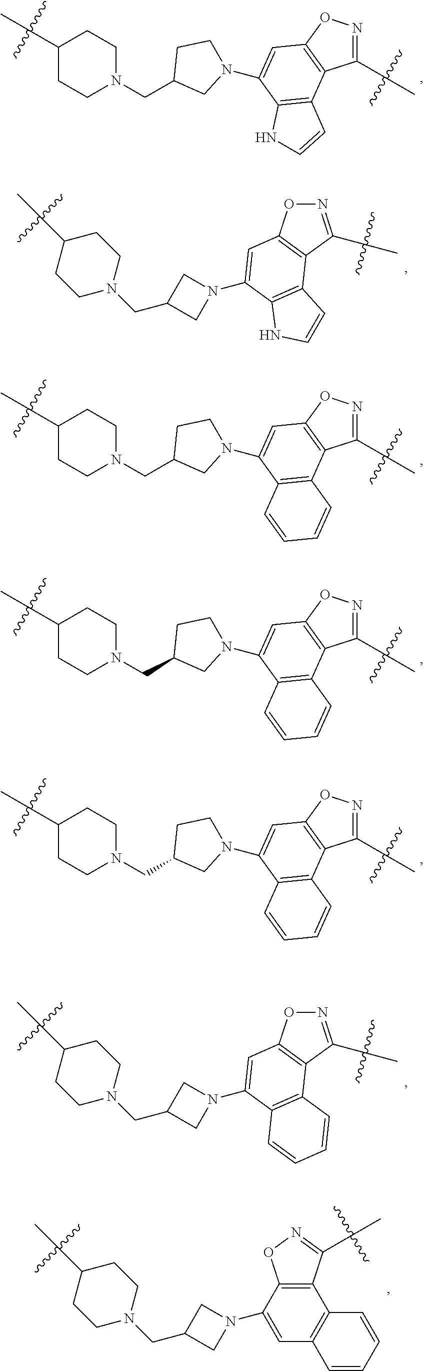

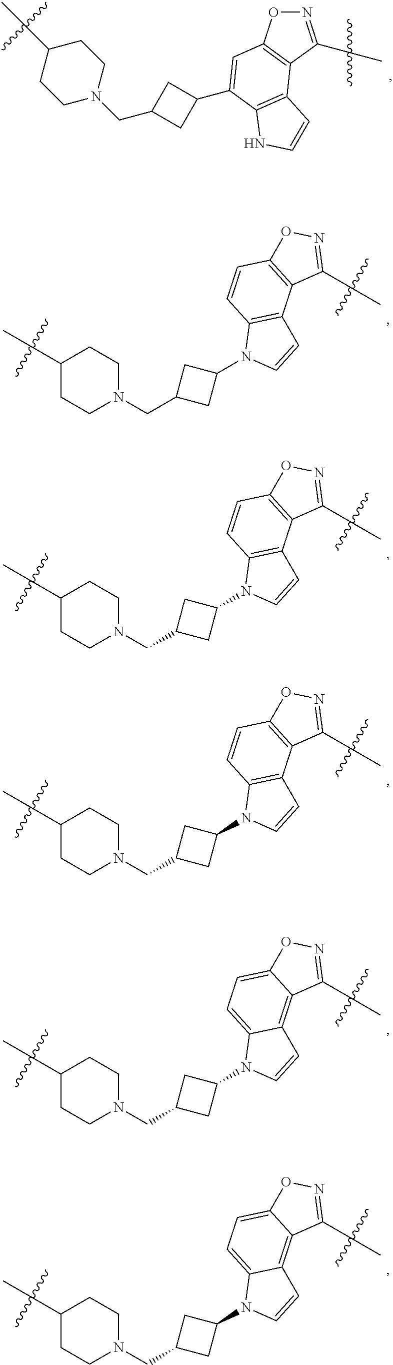

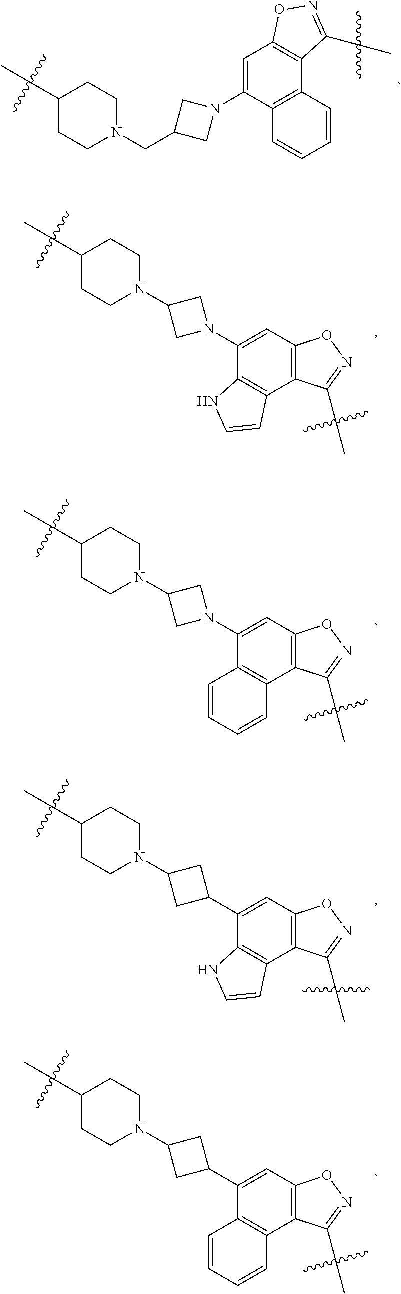 [SMILES: *c1noc2cc(N3CC(CN4CCC(*)CC4)C3)c3ccccc3c12 ; *c1noc2cc(N3CC(N4CCC(*)CC4)C3)c3[nH]ccc3c12 ; *c1noc2cc(N3CC(N4CCC(*)CC4)C3)c3ccccc3c12 ; *c1noc2cc(C3CC(N4CCC(*)CC4)C3)c3[nH]ccc3c12 ; *c1noc2cc(C3CC(N4CCC(*)CC4)C3)c3ccccc3c12]

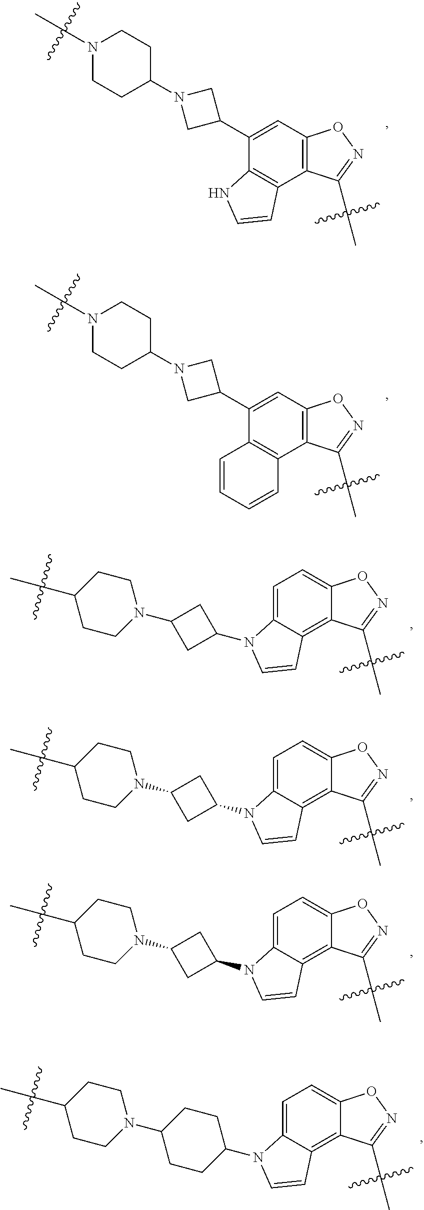 [SMILES: *c1noc2cc(C3CN(C4CCN(*)CC4)C3)c3[nH]ccc3c12 ; *c1noc2cc(C3CN(C4CCN(*)CC4)C3)c3ccccc3c12 ; *c1noc2ccc3c(ccn3C3CC(N4CCC(*)CC4)C3)c12 ; *c1noc2ccc3c(ccn3[C@H]3C[C@@H](N4CCC(*)CC4)C3)c12 ; *c1noc2ccc3c(ccn3[C@H]3C[C@H](N4CCC(*)CC4)C3)c12 ; *c1noc2ccc3c(ccn3C3CCC(N4CCC(*)CC4)CC3)c12]

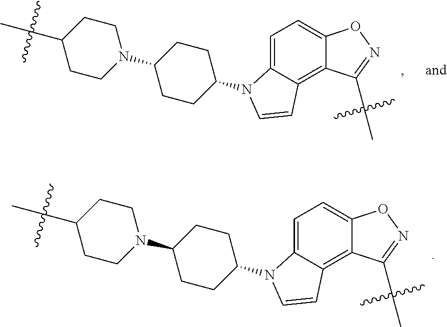 [SMILES: *c1noc2ccc3c(ccn3[C@H]3CC[C@@H](N4CCC(*)CC4)CC3)c12 ; *c1noc2ccc3c(ccn3[C@H]3CC[C@H](N4CCC(*)CC4)CC3)c12]

Alternatively, the moiety

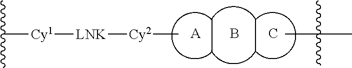

is selected from the group consisting of

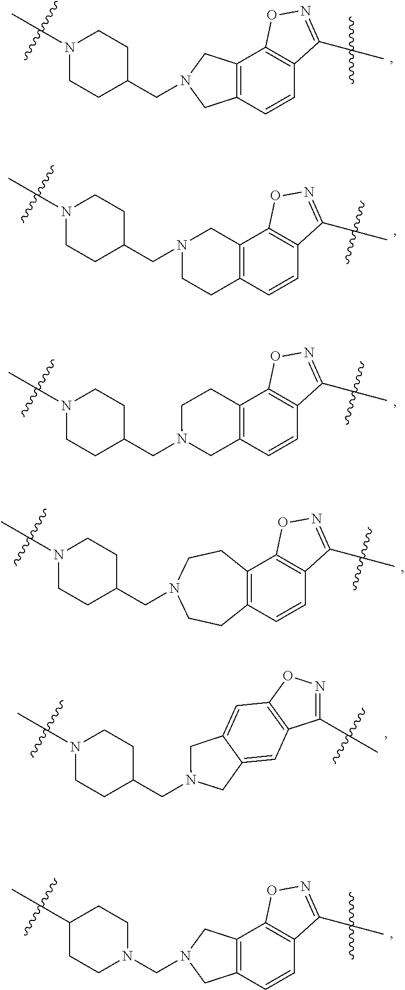 [SMILES: *c1noc2c3c(ccc12)CN(CC1CCN(*)CC1)C3 ; *c1noc2c3c(ccc12)CCN(CC1CCN(*)CC1)C3 ; *c1noc2c3c(ccc12)CN(CC1CCN(*)CC1)CC3 ; *c1noc2c3c(ccc12)CCN(CC1CCN(*)CC1)CC3 ; *c1noc2cc3c(cc12)CN(CC1CCN(*)CC1)C3 ; *c1noc2c3c(ccc12)CN(CN1CCC(*)CC1)C3]

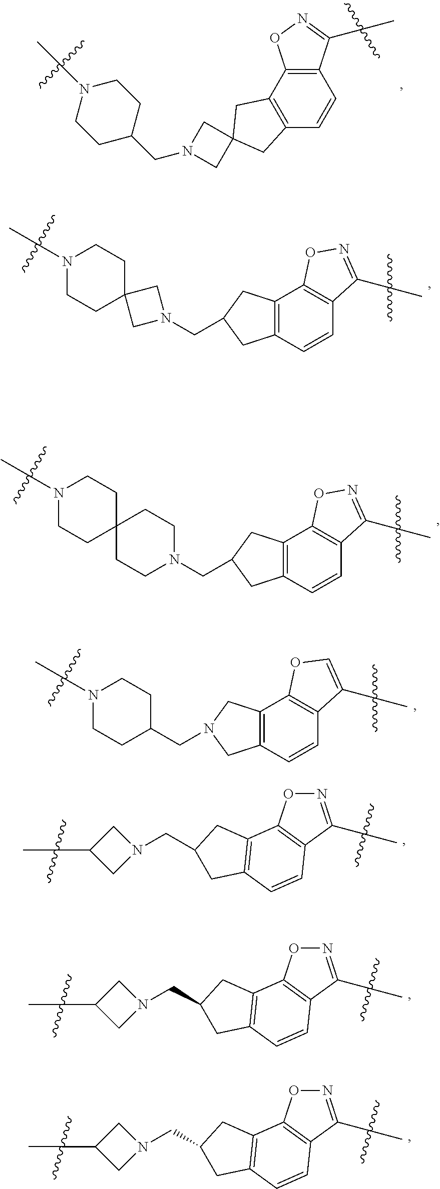

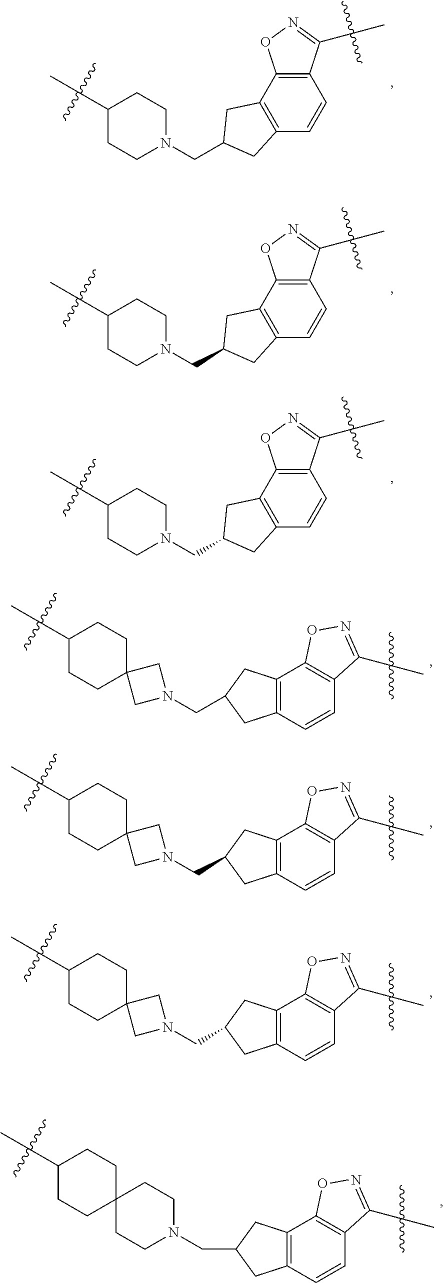

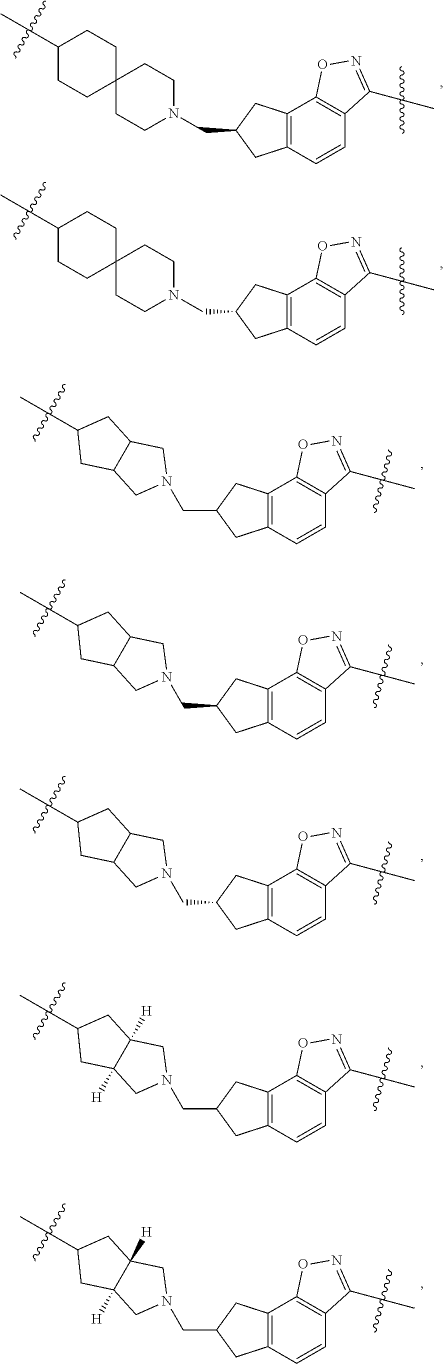

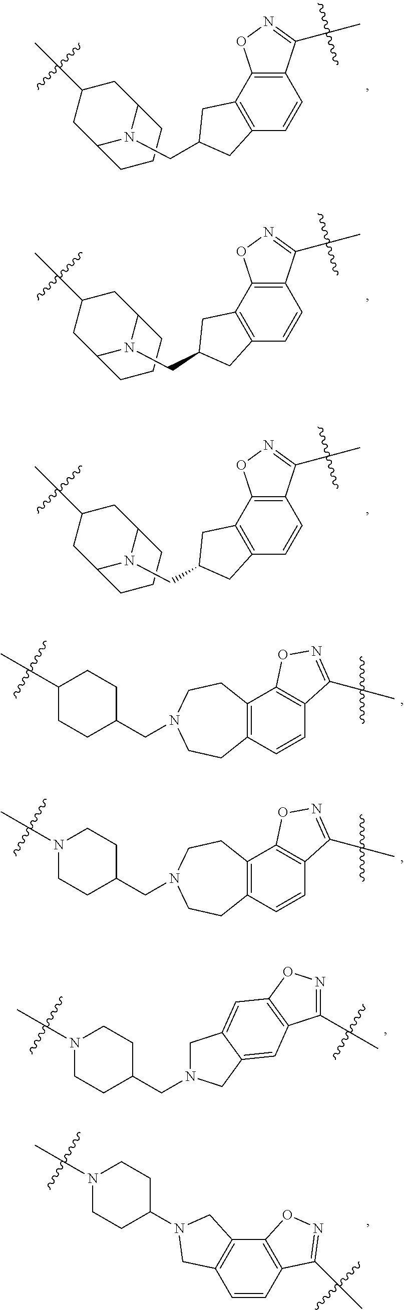

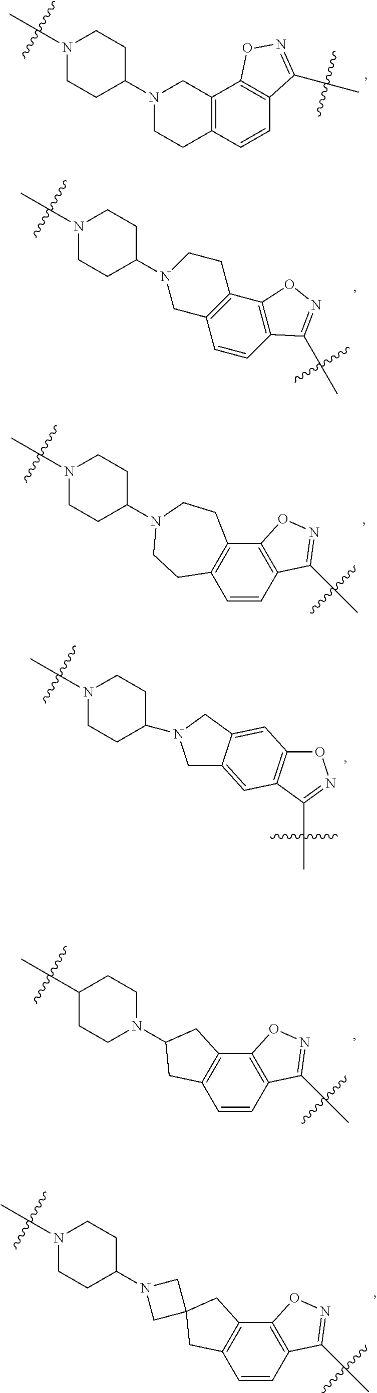 [SMILES: *c1noc2c3c(ccc12)CCN(C1CCN(*)CC1)C3 ; *c1noc2c3c(ccc12)CN(C1CCN(*)CC1)CC3 ; *c1noc2c3c(ccc12)CCN(C1CCN(*)CC1)CC3 ; *c1noc2cc3c(cc12)CN(C1CCN(*)CC1)C3 ; *c1noc2c3c(ccc12)CC(N1CCC(*)CC1)C3 ; *c1noc2c3c(ccc12)CC1(C3)CN(C2CCN(*)CC2)C1]

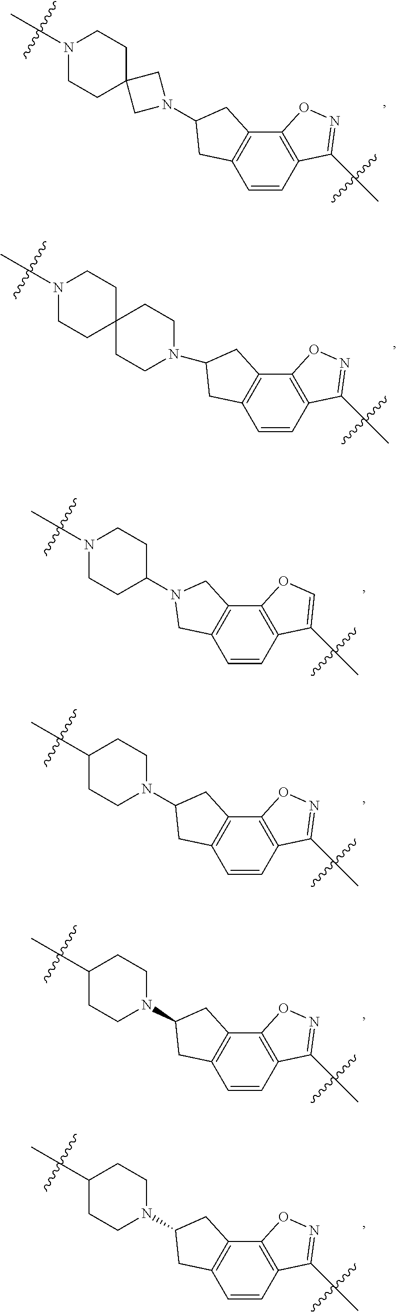 [SMILES: *c1noc2c3c(ccc12)CC(N1CC2(CCN(*)CC2)C1)C3 ; *c1noc2c3c(ccc12)CC(N1CCC2(CCN(*)CC2)CC1)C3 ; *c1coc2c3c(ccc12)CN(C1CCN(*)CC1)C3 ; *c1noc2c3c(ccc12)CC(N1CCC(*)CC1)C3 ; *c1noc2c3c(ccc12)C[C@@H](N1CCC(*)CC1)C3 ; *c1noc2c3c(ccc12)C[C@H](N1CCC(*)CC1)C3]

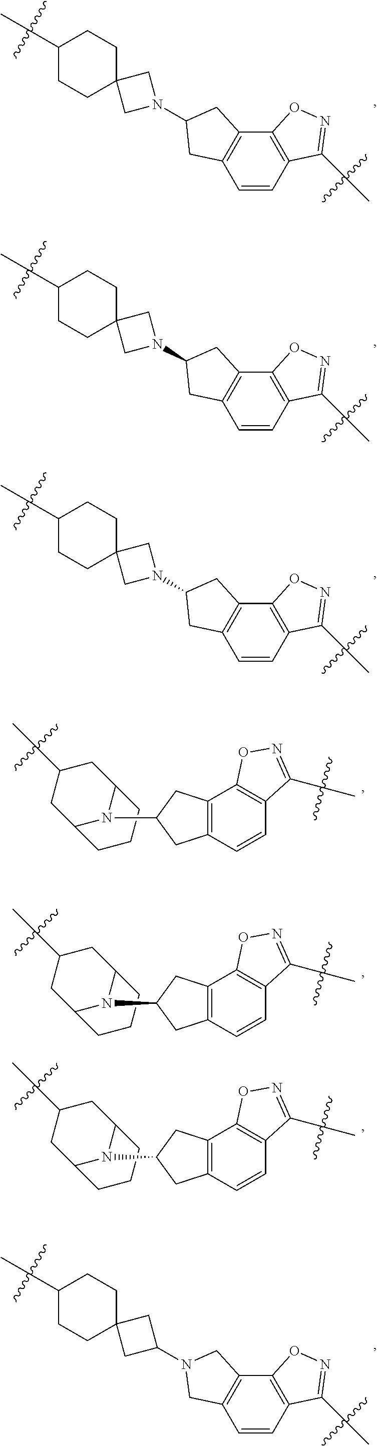

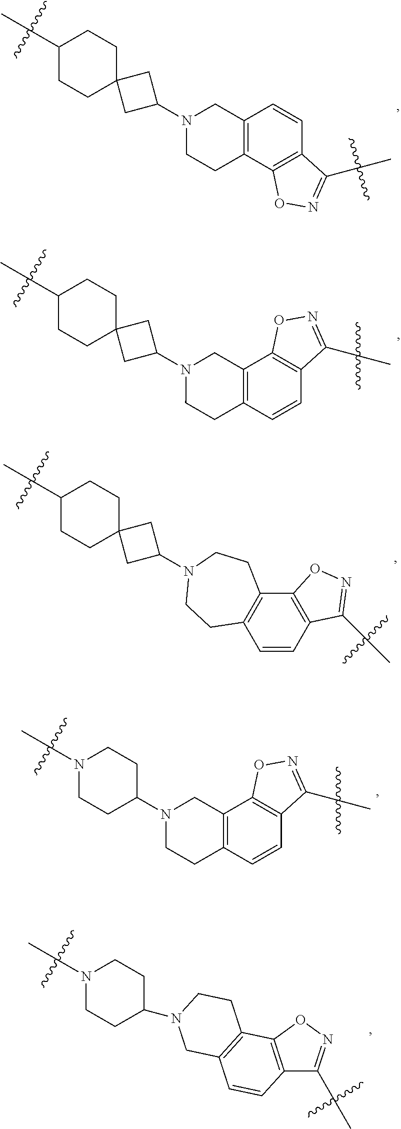 [SMILES: *c1noc2c3c(ccc12)CN(C1CC2(CCC(*)CC2)C1)CC3 ; *c1noc2c3c(ccc12)CCN(C1CC2(CCC(*)CC2)C1)C3 ; *c1noc2c3c(ccc12)CCN(C1CC2(CCC(*)CC2)C1)CC3 ; *c1noc2c3c(ccc12)CCN(C1CCN(*)CC1)C3 ; *c1noc2c3c(ccc12)CN(C1CCN(*)CC1)CC3]

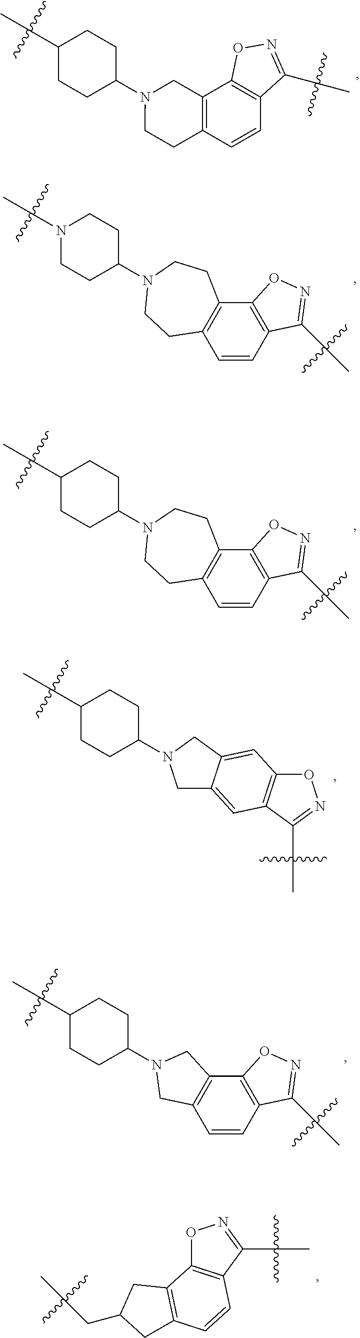 [SMILES: *c1noc2c3c(ccc12)CCN(C1CCC(*)CC1)C3 ; *c1noc2c3c(ccc12)CCN(C1CCN(*)CC1)CC3 ; *c1noc2c3c(ccc12)CCN(C1CCC(*)CC1)CC3 ; *c1noc2cc3c(cc12)CN(C1CCC(*)CC1)C3 ; *c1noc2c3c(ccc12)CN(C1CCC(*)CC1)C3 ; *CC1Cc2ccc3c(*)noc3c2C1]

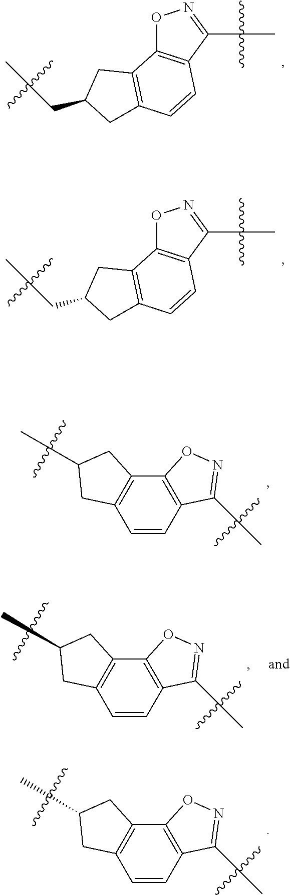 [SMILES: *C[C@@H]1Cc2ccc3c(*)noc3c2C1 ; *C[C@H]1Cc2ccc3c(*)noc3c2C1 ; *c1noc2c3c(ccc12)CC(*)C3 ; *c1noc2c3c(ccc12)C[C@@H](C)C3 ; *c1noc2c3c(ccc12)C[C@H](C)C3]

Further, in some other embodiments, the moiety

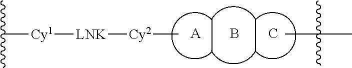

is selected from the group consisting of

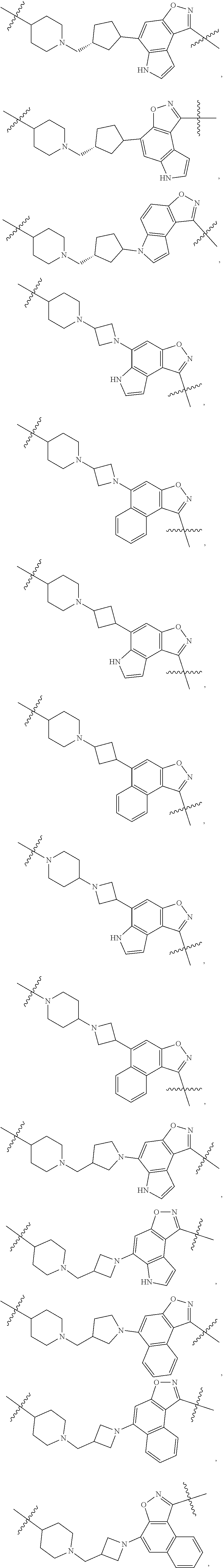

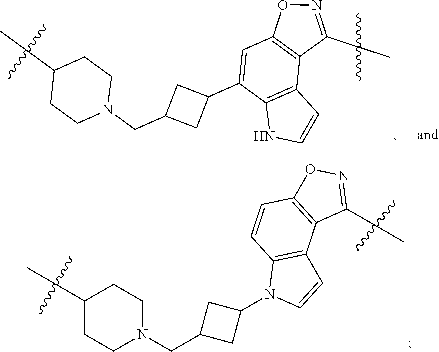

or, the moiety

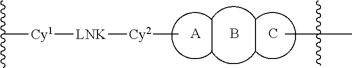

is selected from the group consisting
of

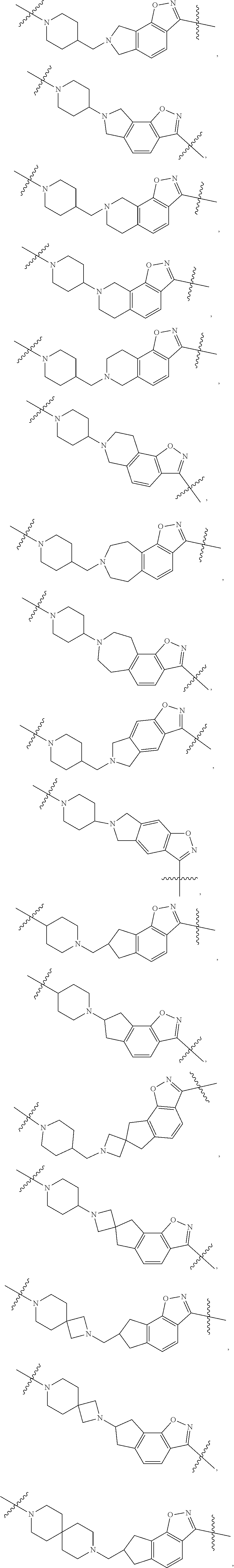

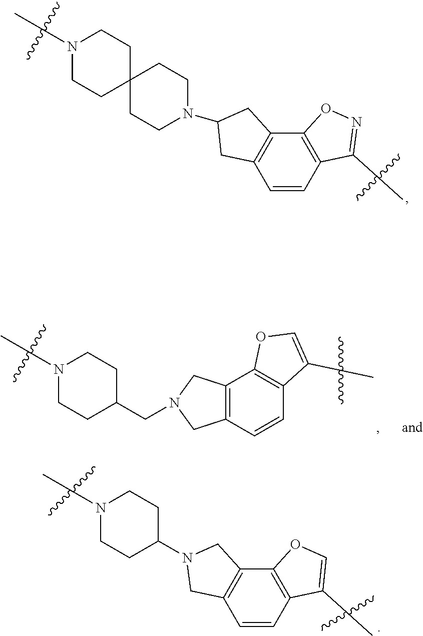 [SMILES: *c1noc2c3c(ccc12)CC(N1CCC2(CCN(*)CC2)CC1)C3 ; *c1coc2c3c(ccc12)CN(CC1CCN(*)CC1)C3 ; *c1coc2c3c(ccc12)CN(C1CCN(*)CC1)C3]

In some embodiments, the PTM is as described herein.

In some embodiments, PTM is selected from

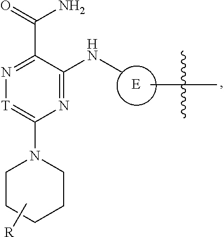

wherein R, T, or ring E is as described herein.

In some embodiments, PTM is selected from

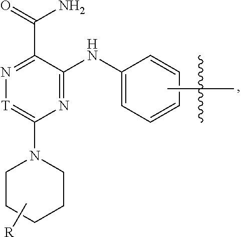

wherein R or T is as described herein.

In another aspect, the present application relates to a compound of formula III below, a moiety, an isomer (e.g., a stereoisomer) thereof, a derivative (such as a Protac molecule) thereof, or a pharmaceutically acceptable salt thereof:

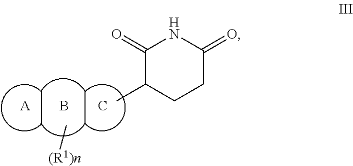

wherein ring A is selected from the group consisting of C₅₋₁₀ cycloalkenyl and 5- to 10-membered heterocycloalkenyl;
ring B is selected from phenyl;
ring C is selected from the group consisting of isoxazolyl and furanyl;
n is as defined herein;
each R¹ is independently selected from the group consisting of halogen, —OH, —NH₂, —CN, ═O, C₁₋₄ alkoxy, —CHO, C₃₋₆ cycloalkyl, 3- to 10-membered heterocycloalkyl, and C₁₋₄ alkyl, wherein the C₃₋₆ cycloalkyl, 3- to 10-membered heterocycloalkyl, or C₁₋₄ alkyl is optionally substituted with halogen, —OH, —NH₂, or C₁₋₄ alkyl-OH.

In some embodiments, the compound of formula III is not selected from the following compound:

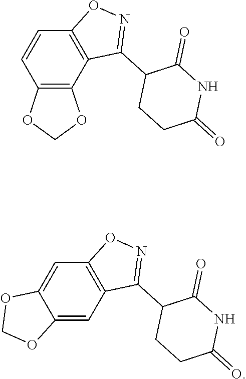 [SMILES: O=C1CCC(c2noc3ccc4c(c23)OCO4)C(=O)N1 ; O=C1CCC(c2noc3cc4c(cc23)OCO4)C(=O)N1]

In some embodiments, the heterocycloalkenyl comprises at least one N atom.

In some embodiments, ring A is selected from the group consisting of C₅₋₈ cycloalkenyl and 5- to 10-membered heterocycloalkenyl; or
ring A is selected from the group consisting of C₅₋₇ cycloalkenyl and 5- to 10-membered heterocycloalkenyl; or
ring A is selected from the group consisting of C₅₋₆ cycloalkenyl and 5- to 10-membered heterocycloalkenyl; or,
ring A is selected from the group consisting of C₅₋₆ cycloalkenyl and 5- to 9-membered heterocycloalkenyl.

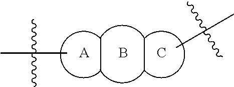

In some embodiments, the structural moiety

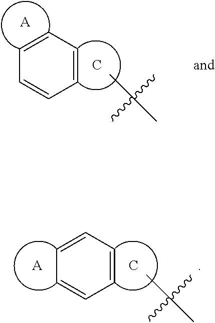

is selected from the group consisting of

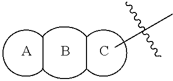

In some embodiments, the structural moiety

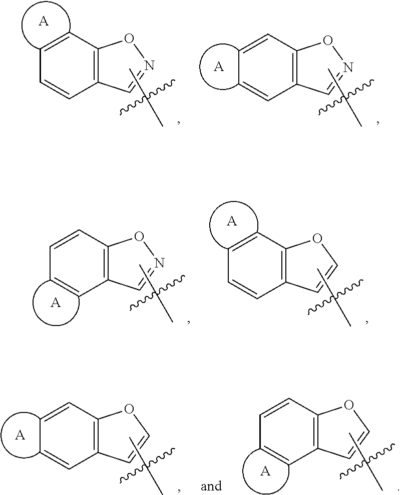

In some embodiments, the structural moiety

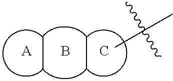

is selected from the group consisting of

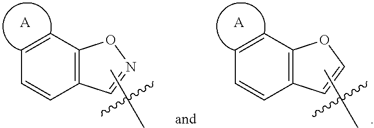

In some embodiments, the structural moiety

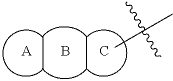

is selected from the group consisting of

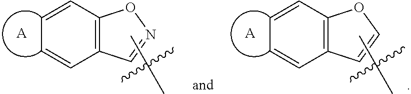

In some embodiments, the structural moiety

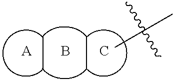

is selected from the group consisting of

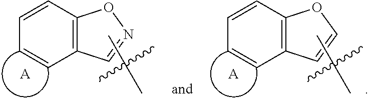

In some embodiments, the structural moiety

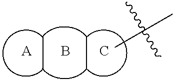

is selected from the group consisting of

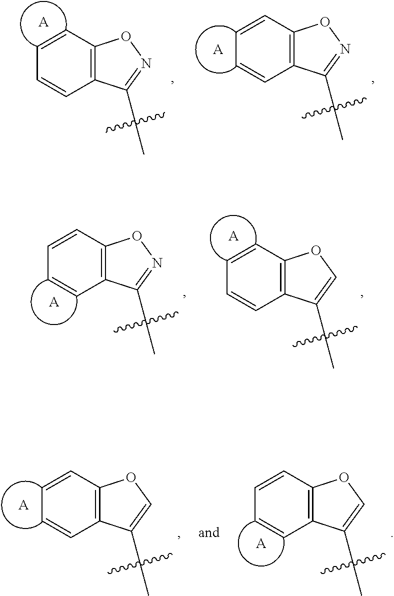

In some specific embodiments, the structural moiety

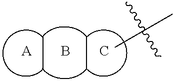

is selected from the group consisting of

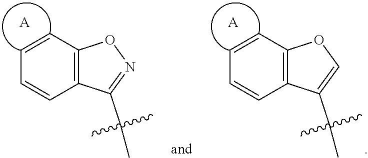

In some specific embodiments, the structural moiety

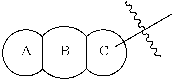

is selected from the group consisting of

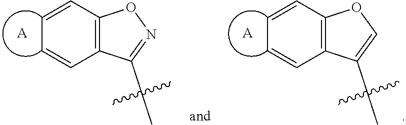

In some specific embodiments, the structural moiety

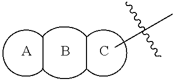

is selected from the group consisting of

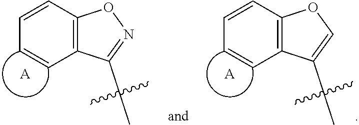

In some embodiments, the structural moiety

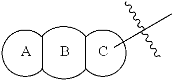

is selected from tile group consisting of

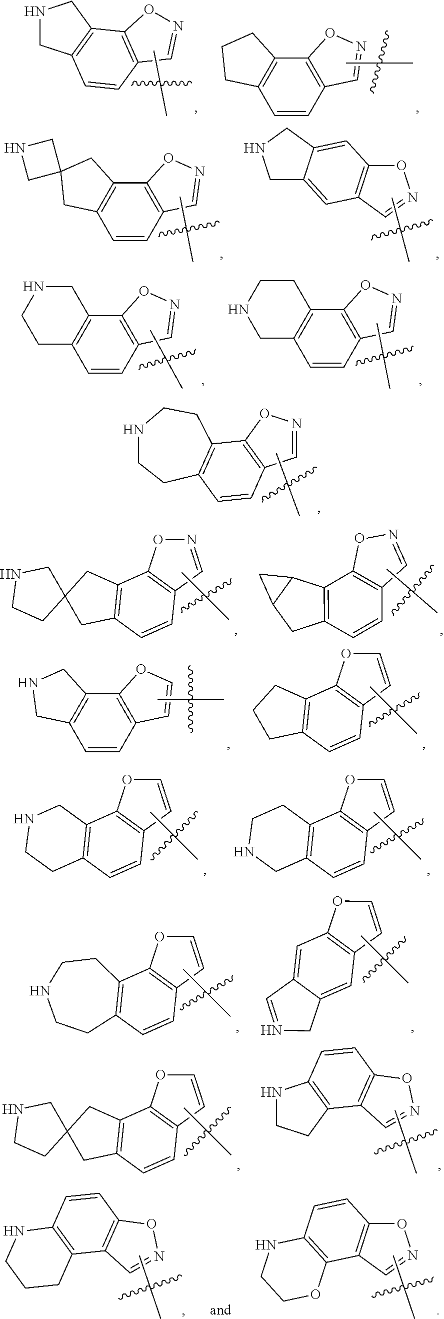

In another aspect, the present application relates to a compound of formula III-1 below, a moiety, an isomer (e.g., a stereoisomer) thereof, a derivative (such as a Protac molecule) thereof, or a pharmaceutically acceptable salt thereof:

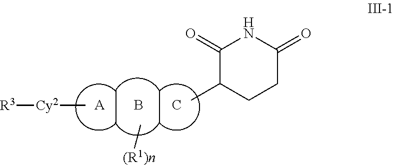

wherein R³ is selected from the group consisting of oxo, hydroxy, —(CH₂)ₘ—CHO, and hydroxy C₁₋₅ alkylene, and
m is 0, 1, 2, 3, 4, or 5, preferably 0; Cy², ring A, ring B, ring C, R¹, and n are as defined herein.

In another aspect, the present application relates to a compound of formula I″′-1a or I″′-2a below, a moiety, an isomer (e.g., a stereoisomer) thereof, a derivative (such as a Protac molecule) thereof, or a pharmaceutically acceptable salt thereof:

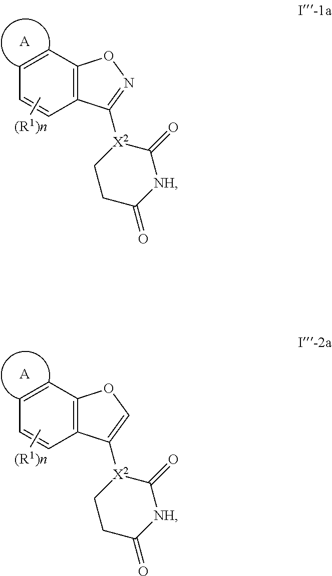

wherein,
ring A is selected from the group consisting of C₅₋₁₀ cycloalkenyl and 5- to 10-membered heterocycloalkenyl; or,
ring A is selected from the group consisting of C₅₋₈ cycloalkenyl and 5- to 10-membered heterocycloalkenyl; or,
ring A is selected from the group consisting of C₅₋₇ cycloalkenyl and 5- to 10-membered heterocycloalkenyl; or,
ring A is selected from the group consisting of C₅₋₆ cycloalkenyl and 5- to 10-membered heterocycloalkenyl;
n is as defined herein;
each R¹ is independently selected from the group consisting of halogen, —OH, —NH₂, —CN, ═O, C₁₋₄ alkoxy, —CHO,
C₃₋₆ cycloalkyl, 3- to 10-membered heterocycloalkyl, and C₁₋₄ alkyl, wherein the C₃₋₆ cycloalkyl, 3- to 10-membered heterocycloalkyl, or C₁₋₄ alkyl is optionally substituted with halogen, —OH, —NH₂, or C₁₋₄ alkyl-OH; or, each R¹ is independently selected from the group consisting of halogen, —OH, —NH₂, —CN, ═O, C₁₋₄ alkoxy, —CHO, C₃₋₆ cycloalkyl, and C₁₋₄ alkyl, wherein the C₃₋₆ cycloalkyl or C₁₋₄ alkyl is optionally substituted with halogen, —OH, —NH₂, or C₁₋₄ alkyl-OH;
X² is selected from the group consisting of CH and N.

In some embodiments, each R¹ is independently selected from the group consisting of halogen, —OH, —NH₂, —CN, ═O, C₁₋₃ alkoxy, —CHO, C₃₋₄ cycloalkyl, and C₁₋₃ alkyl, wherein the C₃₋₄ cycloalkyl or C₁₋₃ alkyl is optionally substituted with halogen, —OH, —NH₂, or C₁₋₃ alkyl-OH.

In some embodiments, each R¹ is independently selected from the group consisting of halogen, —OH, —NH₂, —CN, ═O, methoxy, —CHO, cyclobutyl, and methyl, wherein the cyclobutyl or methyl is optionally substituted with halogen, —OH, —NH₂, or CH₂OH.

In some embodiments, each R¹ is independently selected from the group consisting of F, —OH, —NH₂, —CH₂OH, ═O, —CHO, —CH₂NH₂, and

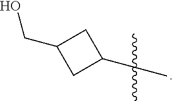 [SMILES: *C1CC(CO)C1]

In some specific embodiments, each R¹ is independently selected from the group consisting of halogen, —OH, —NH₂, —CN, ═O, methoxy, —CHO, and C₁₋₃ alkyl, wherein the C₁₋₃ alkyl is optionally substituted with halogen, —OH, or —NH₂.

In some specific embodiments, each R¹ is independently selected from the group consisting of halogen, —OH, —NH₂, —CN, ═O, methoxy, —CHO, and methyl, wherein the methyl is optionally substituted with —OH or —NH₂.

In some specific embodiments, each R¹ is independently selected from the group consisting of F, —OH, —NH₂, —CH₂OH, ═O, —CHO, and —CH₂NH₂.

In some embodiments, the moiety

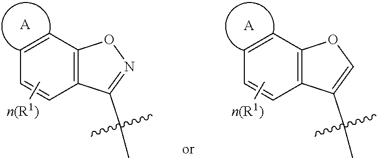

is selected from the group consisting of

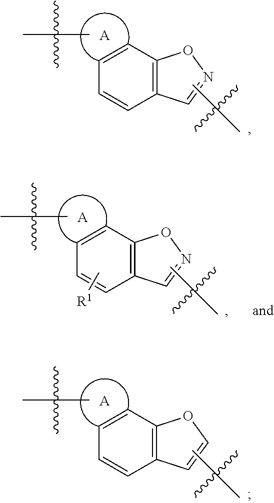

preferably, the moiety is selected from the group consisting of

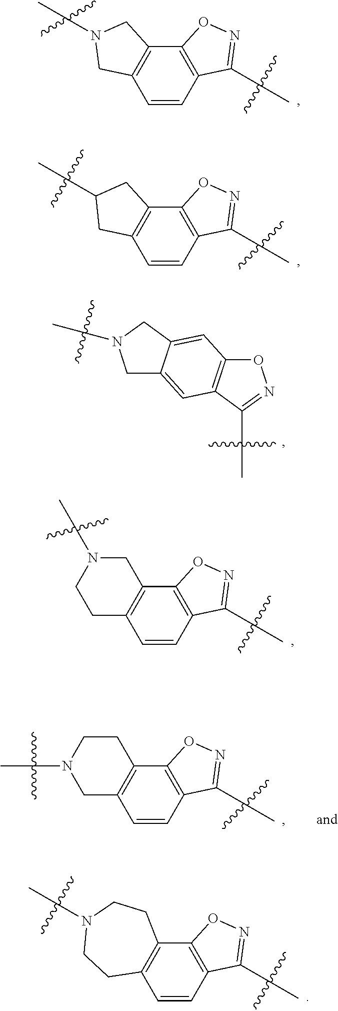 [SMILES: *c1noc2c3c(ccc12)CN(*)C3 ; *c1noc2c3c(ccc12)CC(*)C3 ; *c1noc2cc3c(cc12)CN(*)C3 ; *c1noc2c3c(ccc12)CCN(*)C3 ; *c1noc2c3c(ccc12)CN(*)CC3 ; *c1noc2c3c(ccc12)CCN(*)CC3]

In another aspect, the present application relates to a compound of formula I″′-1 or I″′-2 below, a moiety, an isomer (e.g., a stereoisomer) thereof, a derivative (such as a Protac molecule) thereof, or a pharmaceutically acceptable salt thereof:

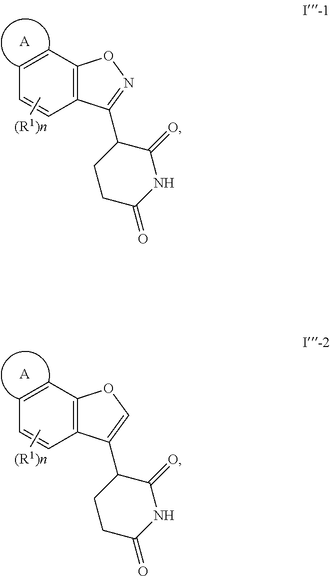

wherein,
ring A is selected from the group consisting of C₅₋₁₀ cycloalkenyl and 5- to 10-membered heterocycloalkenyl; or,
ring A is selected from the group consisting of C₅₋₈ cycloalkenyl and 5- to 10-membered heterocycloalkenyl; or,
ring A is selected from the group consisting of C₅₋₇ cycloalkenyl and 5- to 10-membered heterocycloalkenyl; or,
ring A is selected from the group consisting of C₅₋₆ cycloalkenyl and 5- to 10-membered heterocycloalkenyl; R¹ and n are as defined herein.

In some embodiments, ring A is selected from the group consisting of C₅₋₆ cycloalkenyl and 5- to 9-membered heterocycloalkenyl. In some embodiments, ring A is selected from the group consisting of C₅ cycloalkenyl, C₆ cycloalkenyl, 5-, 6-, 7-, 8-, and 9-membered heterocycloalkenyl.

In some embodiments, ring A is selected from the group consisting of cyclopentenyl, bicyclohexenyl, dihydropyrrolyl, tetrahydropyridinyl, tetrahydroazepinyl, dihydrooxazinyl, azaspirooctenyl, and azaspirononenyl.

In some embodiments, ring A is selected from the group consisting of cyclopentenyl and bicyclohexenyl. In some embodiments, ring A is selected from the group consisting of dihydropyrrolyl, tetrahydropyridinyl, tetrahydroazepinyl, dihydrooxazinyl, azaspirooctenyl, and azaspirononenyl.

In some embodiments, ring A is selected from the group consisting of

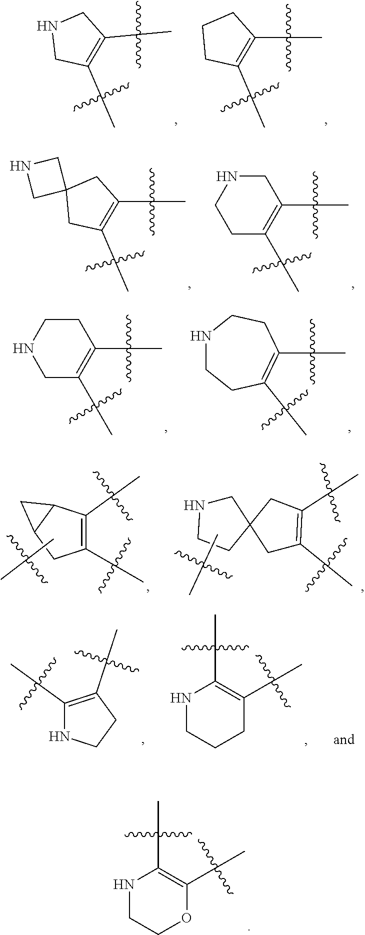

In some embodiments, ring A is selected from the group consisting of

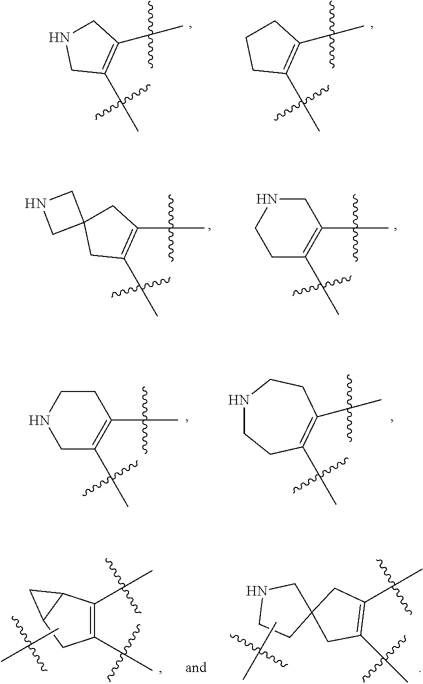

In some other embodiments, ring A is selected from the group consisting of C₅₋₈ cycloalkenyl and 5- to 8-membered heterocycloalkenyl. In some other embodiments, ring A is selected from the group consisting of C₅₋₆ cycloalkenyl and 5- to 8-membered heterocycloalkenyl. In some embodiments, ring A is selected from the group consisting of C₅ cycloalkenyl, 5-, 6-, 7-, and 8-membered heterocycloalkenyl.

In some other embodiments, ring A is selected from the group consisting of cyclopentenyl, dihydropyrrolyl, tetrahydropyridinyl, tetrahydroazepinyl, and azaspirooctene.

In some other embodiments, ring A is selected from cyclopentenyl. In some embodiments, ring A is selected from the group consisting of dihydropyrrolyl, tetrahydropyridinyl, tetrahydroazepinyl, and azaspirooctene.

In some other embodiments, ring A is selected from the group consisting of

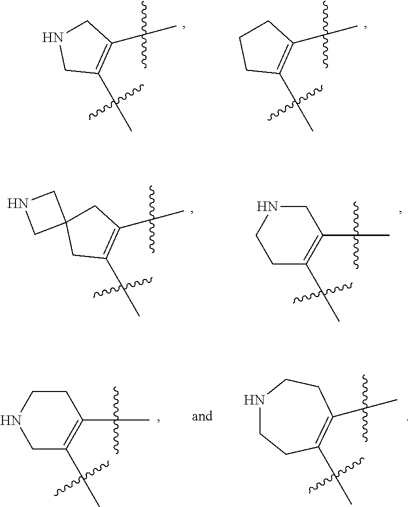 [SMILES: *C1=C(*)CNC1 ; *C1=C(*)CCC1 ; *C1=C(*)CC2(CNC2)C1 ; *C1=C(*)CNCC1 ; *C1=C(*)CNCC1 ; *C1=C(*)CCNCC1]

The present application relates to the following compound, a moiety, a stereoisomer thereof, a derivative (such as a Protac molecule) thereof, or a pharmaceutically acceptable salt thereof:

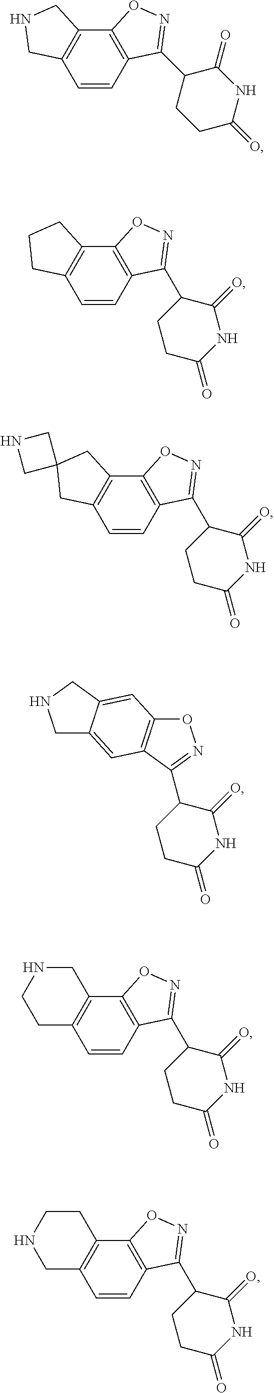 [SMILES: O=C1CCC(c2noc3c4c(ccc23)CNC4)C(=O)N1 ; O=C1CCC(c2noc3c4c(ccc23)CCC4)C(=O)N1 ; O=C1CCC(c2noc3c4c(ccc23)CC2(CNC2)C4)C(=O)N1 ; O=C1CCC(c2noc3cc4c(cc23)CNC4)C(=O)N1 ; O=C1CCC(c2noc3c4c(ccc23)CCNC4)C(=O)N1 ; O=C1CCC(c2noc3c4c(ccc23)CNCC4)C(=O)N1]

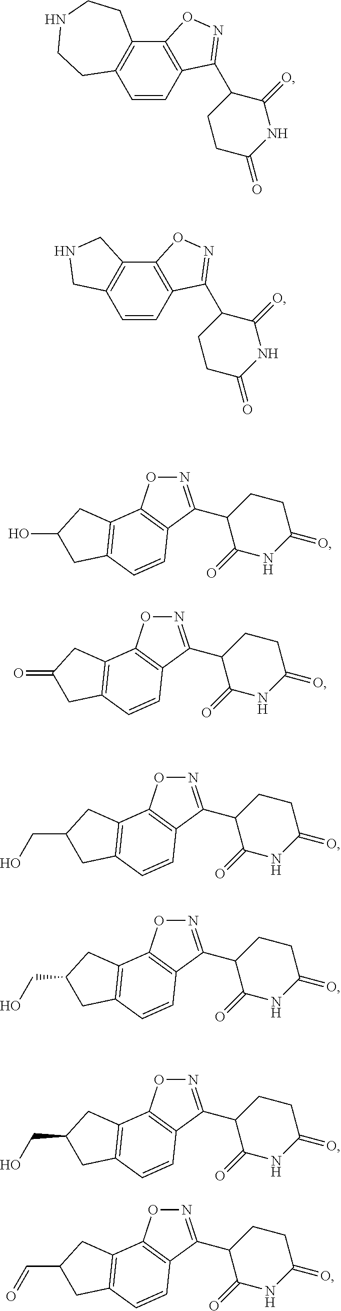

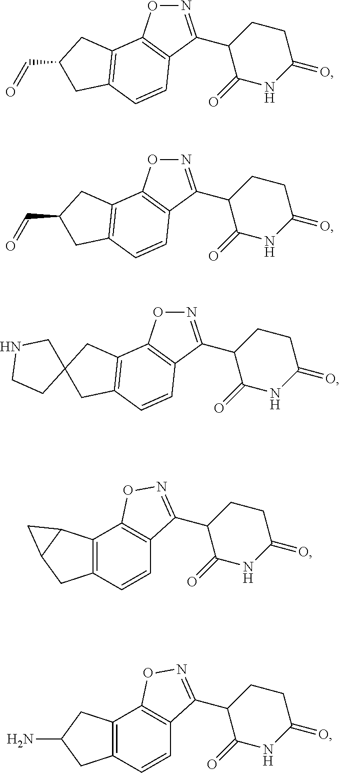 [SMILES: O=C[C@H]1Cc2ccc3c(C4CCC(=O)NC4=O)noc3c2C1 ; O=C[C@@H]1Cc2ccc3c(C4CCC(=O)NC4=O)noc3c2C1 ; O=C1CCC(c2noc3c4c(ccc23)CC2(CCNC2)C4)C(=O)N1 ; O=C1CCC(c2noc3c4c(ccc23)CC2CC42)C(=O)N1 ; NC1Cc2ccc3c(C4CCC(=O)NC4=O)noc3c2C1]

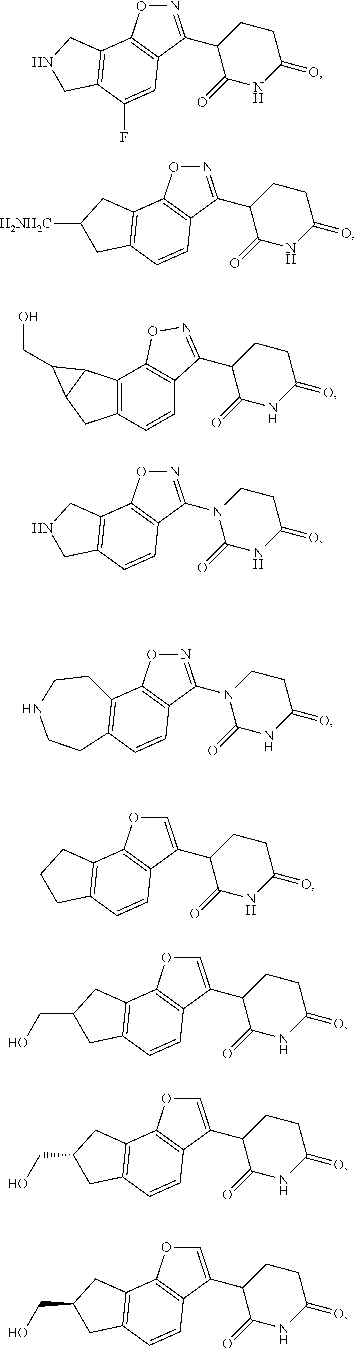

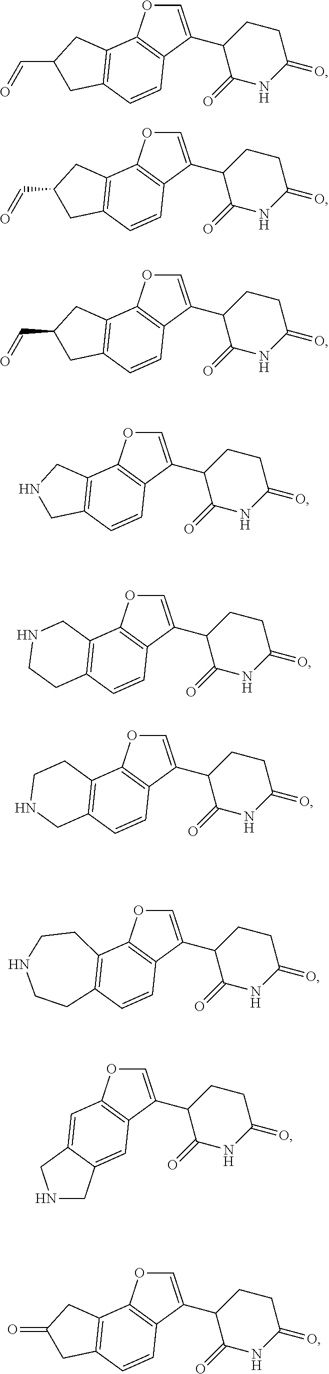

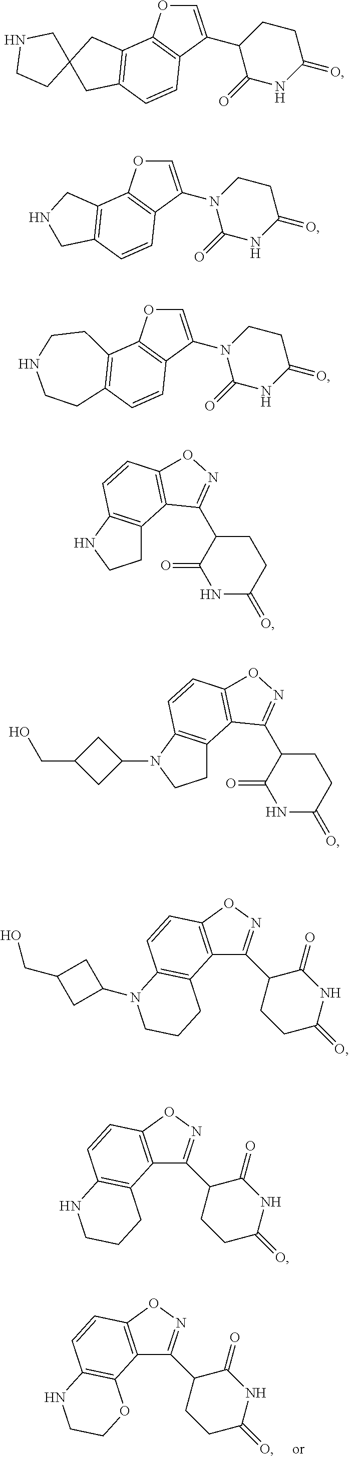

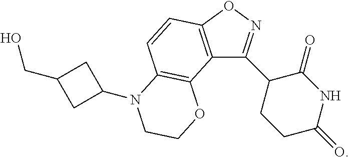 [SMILES: O=C1CCC(c2noc3ccc4c(c23)OCCN4C2CC(CO)C2)C(=O)N1]

In another aspect, the present application relates to a compound of formula I″′-1c or I″′-2d below, a moiety, a stereoisomer thereof, a derivative (such as a Protac molecule) thereof, or a pharmaceutically acceptable salt thereof:

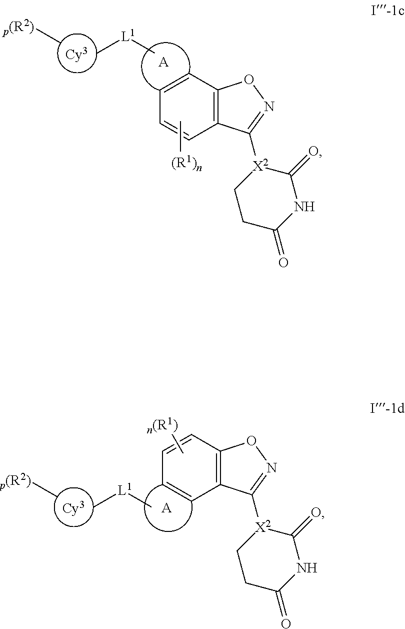

wherein,
ring A, n, and R¹ are as defined herein;
X² is selected from the group consisting of CH and N;
L¹ is selected from C₀₋₃ alkylene;
Cy³ is selected from the group consisting of C₃₋₈ cycloalkyl and 3- to 8-membered heterocycloalkyl;
R² is selected from the group consisting of —CHO, OH, SH, NH₂, COOH, and C₁₋₆ alkyl substituted with one or more SH, OH, or NH₂;
p is selected from the group consisting of 0, 1, 2, and 3.

In some embodiments, ring A is selected from the group consisting of C₅₋₉ cycloalkenyl, 5- to 9-membered heterocycloalkenyl, phenyl, and 5- to 6-membered heteroaryl.

In some embodiments, ring A is selected from the group consisting of C₅₋₇ cycloalkenyl, 5- to 9-membered heterocycloalkenyl, phenyl, and 5- to 6-membered heteroaryl.

In some embodiments, ring A is selected from the group consisting of C₅₋₆ cycloalkenyl, 5- to 9-membered heterocycloalkenyl, phenyl, and 5- to 6-membered heteroaryl.

In some embodiments, ring A is selected from the group consisting of 5- to 6-membered heterocycloalkenyl, phenyl, and 5-membered heteroaryl.

In some embodiments, ring A is selected from the group consisting of dihydropyrrolyl, tetrahydropyridinyl, dihydrooxazinyl, phenyl, pyrrolyl, pyrazolyl, and furanyl.

In some embodiments, the moiety

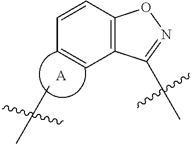

is selected from the group consisting of

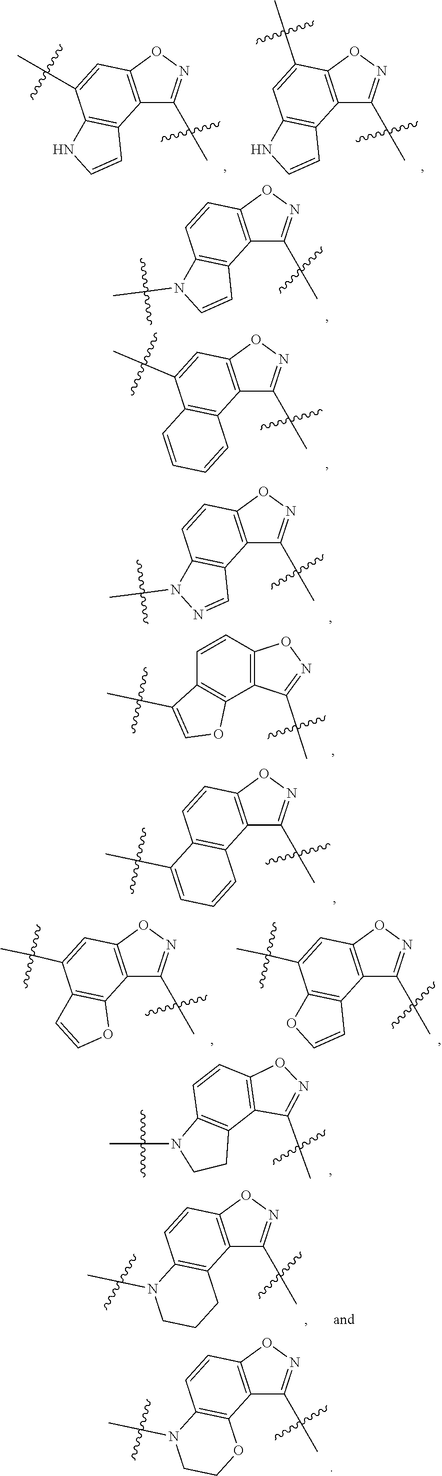

In some embodiments, L¹ is selected from the group consisting of a bond and —CH₂—.

In some embodiments, Cy³ is selected from the group consisting of C₃₋₆ cycloalkyl and 4- to 6-membered heterocycloalkyl.

In some embodiments, Cy³ is selected from the group consisting of C₄₋₆ cycloalkyl, 4-membered heterocycloalkyl, and 6-membered heterocycloalkyl.

In some embodiments, Cy³ is selected from the group consisting of cyclobutyl, cyclopentyl, cyclohexyl, azetidinyl, and piperidinyl.

In some embodiments, R² is selected from the group consisting of —CHO, OH, and C₁₋₃ alkyl substituted with one or more OH or NH₂.

In some embodiments, R² is selected from the group consisting of —CHO, OH, and methyl substituted with one or more OH.

In some embodiments, R² is selected from the group consisting of —CHO, OH, and —CH₂OH.

In some embodiments, X² is selected from CH.

In some embodiments, p is selected from the group consisting of 0, 1, and 2.

In some embodiments, p is selected from the group consisting of 0 and 1. In some embodiments, p is selected from 0. In some embodiments, p is selected from 1.

In some embodiments, the structural moiety

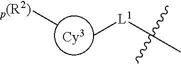

is selected from the group consisting of

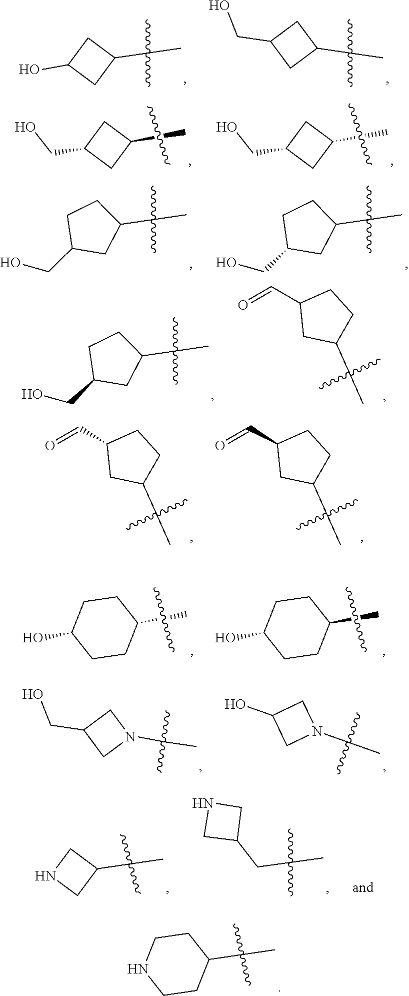

The present application relates to the following compound, a moiety, a stereoisomer thereof, a derivative (such as a Protac molecule) thereof, or a pharmaceutically acceptable salt thereof.

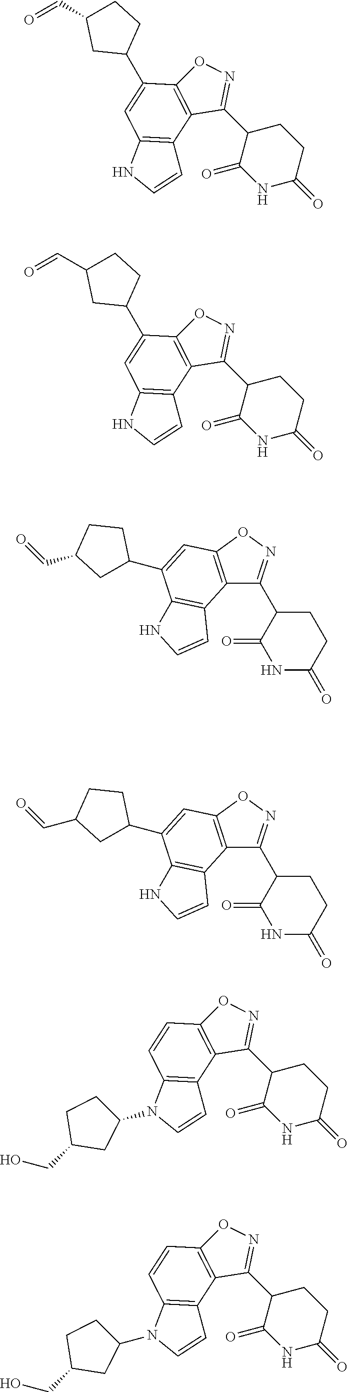 [SMILES: O=C[C@@H]1CCC(c2cc3[nH]ccc3c3c(C4CCC(=O)NC4=O)noc23)C1 ; O=CC1CCC(c2cc3[nH]ccc3c3c(C4CCC(=O)NC4=O)noc23)C1 ; O=C[C@@H]1CCC(c2cc3onc(C4CCC(=O)NC4=O)c3c3cc[nH]c23)C1 ; O=CC1CCC(c2cc3onc(C4CCC(=O)NC4=O)c3c3cc[nH]c23)C1 ; O=C1CCC(c2noc3ccc4c(ccn4[C@H]4CC[C@@H](CO)C4)c23)C(=O)N1 ; O=C1CCC(c2noc3ccc4c(ccn4C4CC[C@@H](CO)C4)c23)C(=O)N1]

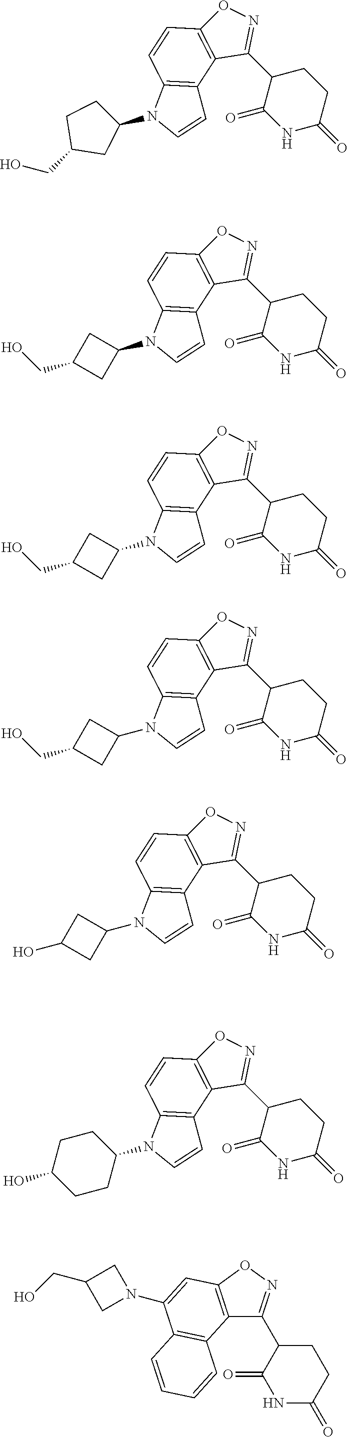

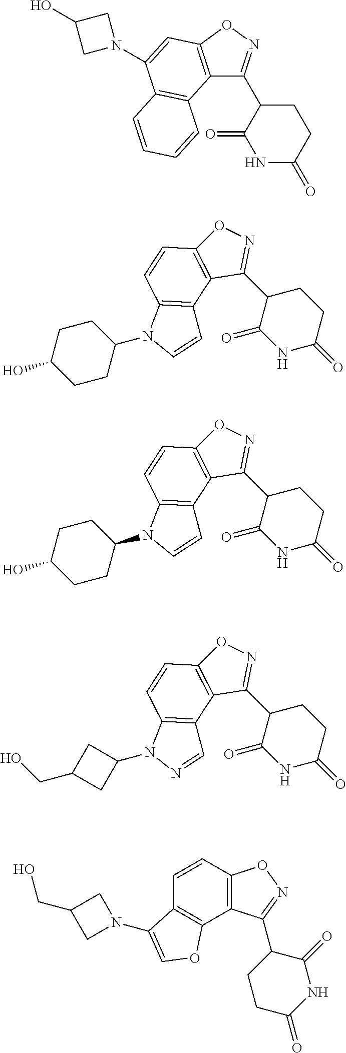

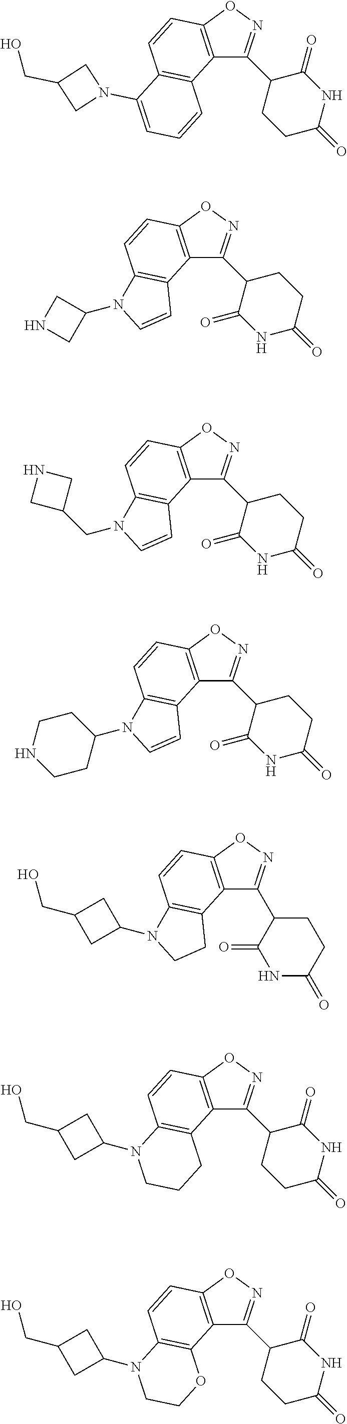

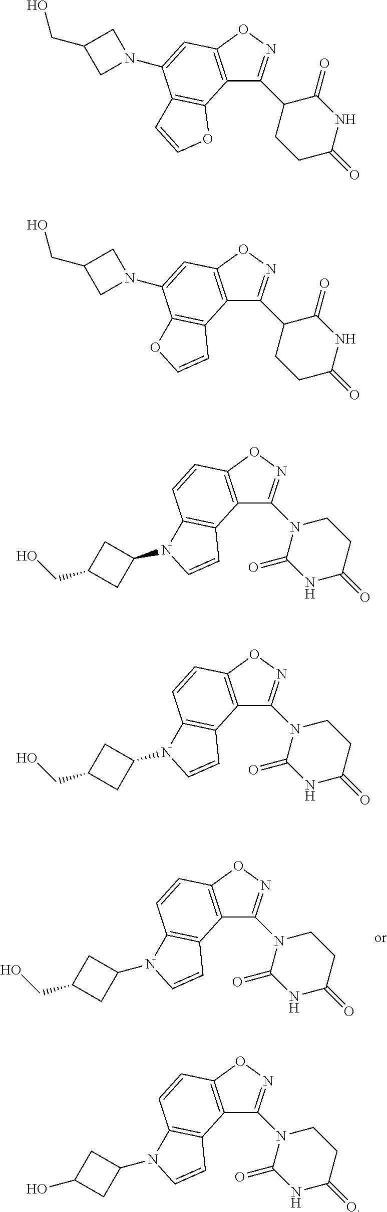

The present application relates to the following compound, a moiety, a stereoisomer thereof, a derivative (such as a Protac molecule) thereof, or a pharmaceutically acceptable salt thereof,

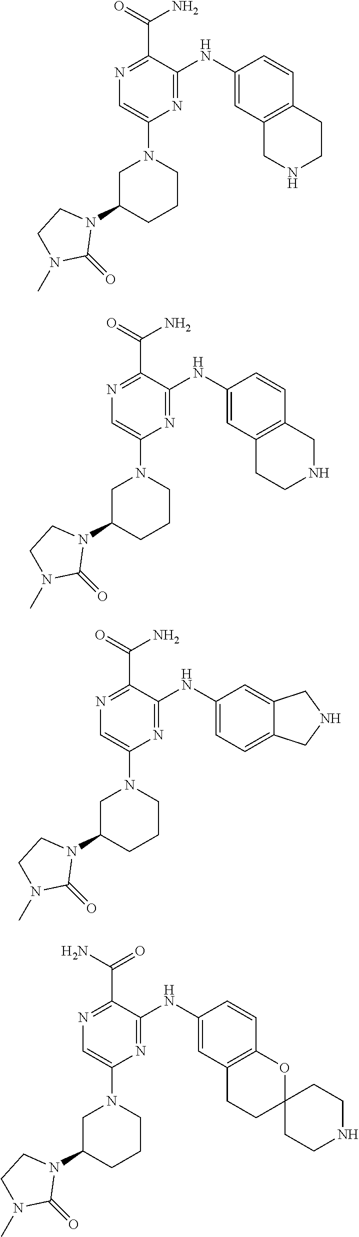

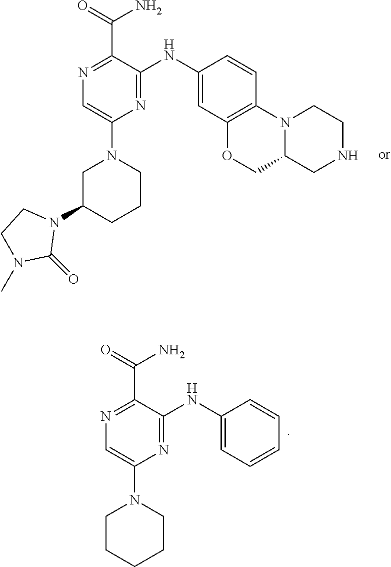 [SMILES: CN1CCN([C@@H]2CCCN(c3cnc(C(N)=O)c(Nc4ccc5c(c4)OC[C@H]4CNCCN54)n3)C2)C1=O ; "or" ; NC(=O)c1ncc(N2CCCCC2)nc1Nc1ccccc1]

In another aspect, the present application relates to use of the compound (e.g., formula III, III-1, I″′-1a, I″′-2a, I″′1, I″′-2, I″′-1c, I″′-2d, or a specific compound), the moiety, the isomer (e.g., a stereoisomer) thereof, and the derivative thereof in a Protac molecule. In another aspect, the present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof for constituting part of a Protac molecule. In another aspect, the present application relates to the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof present in the form of a Protac molecule. In another aspect, the present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof for degrading a protein. For example, the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof degrade the protein in the form of a Protac molecule. In another aspect, the present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof for degrading a protein in the form of a Protac molecule. The present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof (e.g., as a preparation intermediate) for preparing a Protac molecule. The present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof (e.g., as a preparation intermediate) for preparing a protein degrader. Optionally, the Protac molecule may not include a Protac molecule that relates to AR.

Specifically, for example, in some embodiments, the present application relates to use of the following compound, a moiety thereof, a stereoisomer thereof, or a pharmaceutically acceptable salt thereof for preparing a Protac molecule:

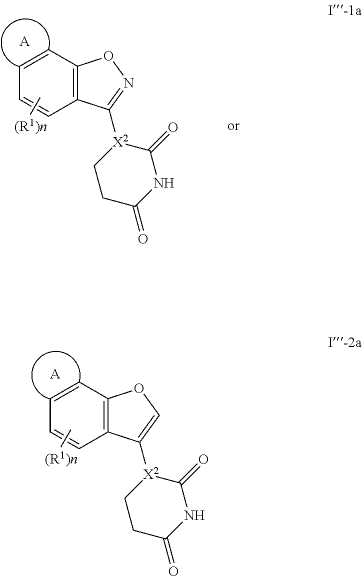

wherein,
ring A is selected from the group consisting of C₅₋₁₀ cycloalkenyl and 5- to 10-membered heterocycloalkenyl; or,
ring A is selected from the group consisting of C₅₋₈ cycloalkenyl and 5- to 10-membered heterocycloalkenyl; or,
ring A is selected from the group consisting of C₅₋₇ cycloalkenyl and 5- to 10-membered heterocycloalkenyl; or,
ring A is selected from the group consisting of C₅₋₆ cycloalkenyl and 5- to 10-membered heterocycloalkenyl; preferably, ring A is selected from the group consisting of cyclopentenyl, dihydropyrrolyl, tetrahydropyridinyl, and tetrahydroazepinyl;
n is selected from the group consisting of 0, 1, 2, and 3; preferably, n is selected from the group consisting of 0, 1, and 2;
each R¹ is independently selected from the group consisting of halogen, —OH, —NH₂, —CN, ═O, C₁₋₄ alkoxy, —CHO, C₃₋₆ cycloalkyl, and C₁₋₄ alkyl, wherein the C₃₋₆ cycloalkyl or C₁₋₄ alkyl is optionally substituted with halogen, —OH, —NH₂, or C₁₋₄ alkyl-OH; preferably, each R¹ is independently selected from the group consisting of fluorine, chlorine, and bromine;
X² is selected from the group consisting of CH and N; preferably, X² is CH;
the moiety of the compound may be

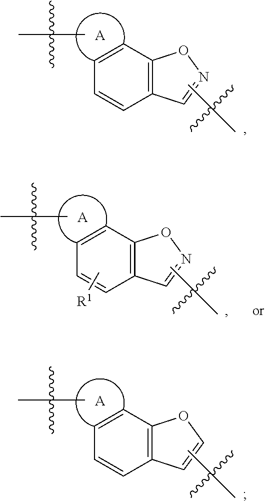

preferably, the moiety is selected from the group consisting of

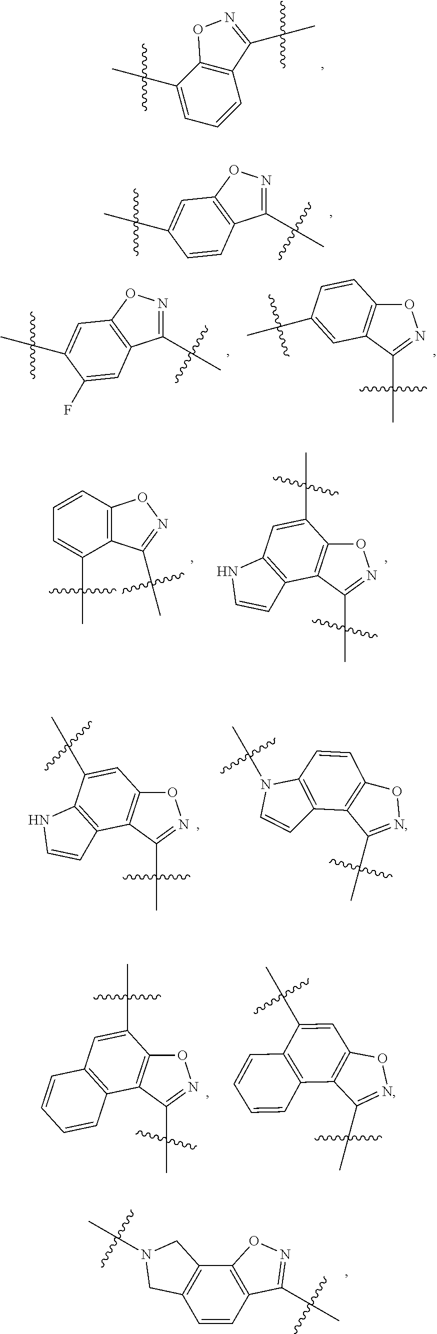

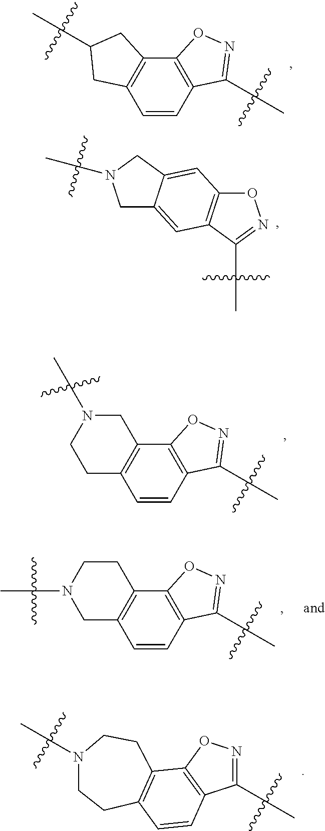

In some embodiments, the present application relates to a method for preparing a Protac molecule, comprising: reacting the following compound, a moiety thereof, a stereoisomer thereof, or a pharmaceutically acceptable salt thereof to prepare the Protac molecule:

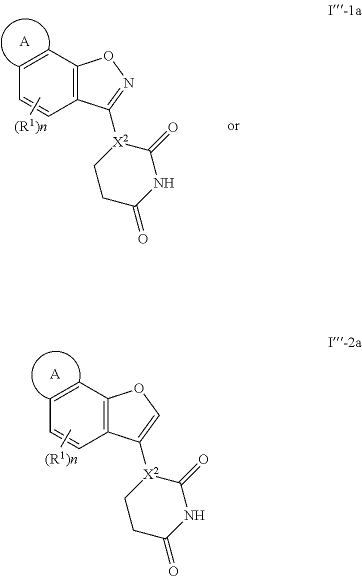

wherein,
ring A is selected from the group consisting of C₅₋₁₀ cycloalkenyl and 5- to 10-membered heterocycloalkenyl; or,
ring A is selected from the group consisting of C₅₋₈ cycloalkenyl and 5- to 10-membered heterocycloalkenyl; or,
ring A is selected from the group consisting of C₅₋₇ cycloalkenyl and 5- to 10-membered heterocycloalkenyl; or,
ring A is selected from the group consisting of C₅₋₆ cycloalkenyl and 5- to 10-membered heterocycloalkenyl;
preferably, ring A is selected from the group consisting of cyclopentenyl, dihydropyrrolyl, tetrahydropyridinyl, and tetrahydroazepinyl;
n is selected from the group consisting of 0, 1, 2, and 3; preferably, n is selected from the group consisting of 0, 1, and 2;
each R¹ is independently selected from the group consisting of halogen, —OH, —NH₂, —CN, ═O, C₁₋₄ alkoxy, —CHO, C₃₋₆ cycloalkyl, and C₁₋₄ alkyl, wherein the C₃₋₆ membered cycloalkyl or C₁₋₄ alkyl is optionally substituted with halogen, —OH, —NH₂, or C₁₋₄ alkyl-OH; preferably, each R¹ is independently selected from the group consisting of fluorine, chlorine, and bromine;
X² is selected from the group consisting of CH and N; preferably, X² is CH;
the moiety of the compound may be

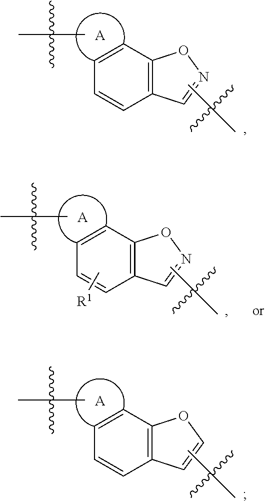

preferably, the moiety is selected from the group consisting of

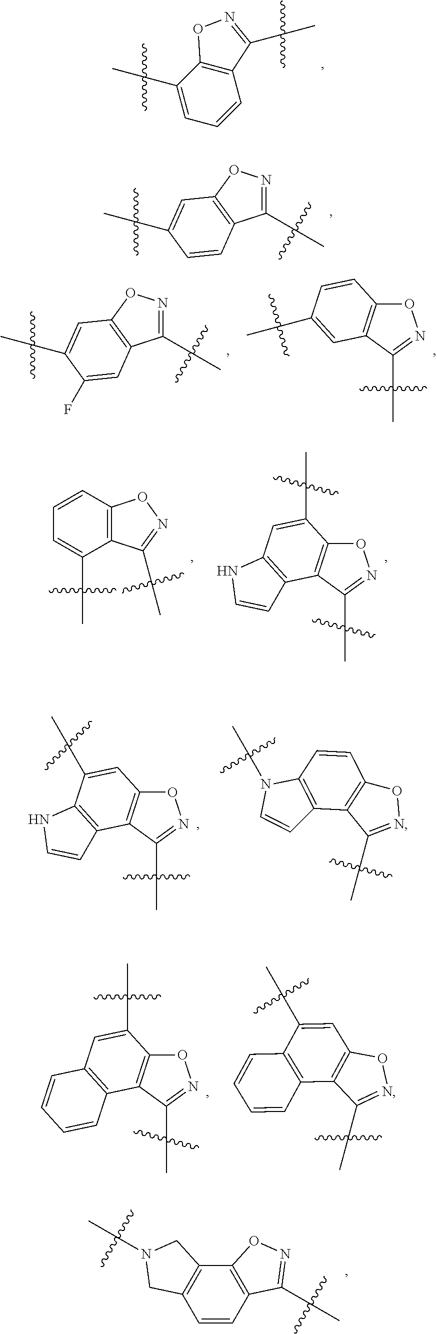

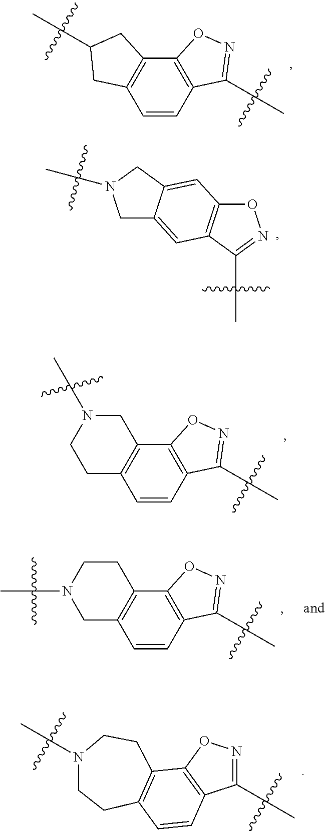 [SMILES: *c1noc2c3c(ccc12)CC(*)C3 ; *c1noc2cc3c(cc12)CN(*)C3 ; *c1noc2c3c(ccc12)CCN(*)C3 ; *c1noc2c3c(ccc12)CN(*)CC3 ; *c1noc2c3c(ccc12)CCN(*)CC3]

The present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof for degrading BTK protein. For example, the compound, the moiety, the isomer (e.g., a stereoisomer) thereof, and the derivative thereof degrade the BTK protein in the form of a Protac molecule. In another aspect, the present application relates to use of the compound (e.g., formula III, III-1, I″′-1a, I″′-2a, I″′-1, I″′-2, I″′-1c, I″′-2d, or a specific compound), the moiety, the isomer (e.g., a stereoisomer) thereof, and the derivative thereof for degrading BTK protein in the form of a Protac molecule.

In some embodiments, the Protac molecule or protein degrader is selected from a BTK protein degrader/molecule.

In another aspect, the present application relates to a pharmaceutical composition comprising the compound, the moiety, the stereoisomer thereof, the derivative thereof, or the pharmaceutically acceptable salt thereof of the present application described above. The pharmaceutical composition of the present application also comprises a pharmaceutically acceptable excipient.

In another aspect, the present application relates to use of the compound, the moiety, the stereoisomer thereof, the derivative thereof or the pharmaceutically acceptable salt thereof, or the pharmaceutical composition thereof described above for preparing a medicament for preventing or treating a disorder treated by degrading a target protein that binds to a targeting ligand.

In another aspect, the present application relates to use of the compound, the moiety, the stereoisomer thereof, the derivative thereof or the pharmaceutically acceptable salt thereof, or the pharmaceutical composition thereof described above for preparing a medicament for preventing or treating a disorder treated by binding to cereblon protein in vivo.

In another aspect, the present application relates to use of the compound, the moiety, the stereoisomer thereof, the derivative thereof or the pharmaceutically acceptable salt thereof, or the pharmaceutical composition thereof described above for preparing a medicament for preventing or treating a BTK-related disease.

The present application relates to a method for treating or preventing a disorder treated by degrading a target protein that binds to a targeting ligand in a mammal, comprising administering to a mammal, preferably a human, in need of such treatment a therapeutically effective amount of the compound, the stereoisomer thereof, the derivative thereof or the pharmaceutically acceptable salt thereof, or the pharmaceutical composition thereof of the present application described above.

The present application relates to a method for treating or preventing a disorder treated by binding to cereblon protein in vivo, comprising administering to a mammal, preferably a human, in need of such treatment a therapeutically effective amount of the compound, the stereoisomer thereof, the derivative thereof or the pharmaceutically acceptable salt thereof, or the pharmaceutical composition thereof of the present application described above.

In another aspect, the present application relates to a method for treating a BTK-related disease in a mammal, comprising administering to a mammal, preferably a human, in need of such treatment a therapeutically effective amount of the compound, the stereoisomer thereof, the derivative thereof or the pharmaceutically acceptable salt thereof, or the pharmaceutical composition thereof of the present application described above.

In another aspect, the present application relates to the compound, the moiety, the stereoisomer thereof, the derivative thereof or the pharmaceutically acceptable salt thereof, or the pharmaceutical composition thereof described above for use in preventing or treating a disorder treated by degrading a target protein that binds to a targeting ligand.

In another aspect, the present application relates to the compound, the moiety, the stereoisomer thereof, the derivative thereof or the pharmaceutically acceptable salt thereof, or the pharmaceutical composition thereof described above for use in preventing or treating a disorder treated by binding to cereblon protein in vivo.

In another aspect, the present application relates to the compound, the moiety, the stereoisomer thereof, the derivative thereof or the pharmaceutically acceptable salt thereof, or the pharmaceutical composition thereof described above for use in preventing or treating a BTK-related disease.

In another aspect, the present application relates to use of the compound, the moiety, the stereoisomer thereof, the derivative thereof or the pharmaceutically acceptable salt thereof, or the pharmaceutical composition thereof described above for preventing or treating a disorder treated by degrading a target protein that binds to a targeting ligand.

In another aspect, the present application relates to use of the compound, the moiety, the stereoisomer thereof, the derivative thereof or the pharmaceutically acceptable salt thereof, or the pharmaceutical composition thereof described above for preventing or treating a disorder treated by binding to cereblon protein in vivo.

In another aspect, the present application relates to use of the compound, the moiety, the stereoisomer thereof, the derivative thereof or the pharmaceutically acceptable salt thereof, or the pharmaceutical composition thereof described above for preventing or treating a BTK-related disease. In some specific embodiments, the BTK-related disease described above is selected from a disorder treated by degrading a protein that binds to a ligand of a BTK target protein; in some specific embodiments, the BTK-related disease described above is selected from a disorder treated by binding to cereblon protein in vivo; in some embodiments, the disease or disorder described above is selected from the group consisting of autoimmune diseases, inflammatory diseases, and cancer.

In some specific embodiments, the disorder treated by binding to cereblon protein in vivo and/or the disorder treated by binding to cereblon protein in vivo described above is selected from a BTK-related disease; in some specific embodiments, the BTK-related disease described above is selected from the group consisting of autoimmune diseases, inflammatory diseases, and cancer.

In some embodiments, the “one or more” is selected from the group consisting of one, two, three, four, five, and six. In some embodiments, the “one or more” is selected from the group consisting of one, two, and three. In some embodiments, the “one or more” is selected from the group consisting of one and two.

In some embodiments, the present application encompasses the variables defined above and embodiments thereof, as well as any combination thereof.

### Technical Effects

The compound of the present application has a degradation effect on BTK of OCI-LY10 cells, can inhibit cell (OCI-LY10 cells, TMD8-BTK^{C481S} cells, or OCI-LY10-BTK^{C481S} cells) proliferation in vitro, has selectivity for BTK and/or BTK^{C481S} kinase as compared to EGFR and TEC kinases, has stable in vitro metabolism and good in vivo pharmacokinetic properties, has an inhibition effect on mouse TMD-8 xenograft tumor in vivo, has binding activity to CRBN protein, and can exhibit the desired IKZF1, IKZF3, and GSPT1 protein degradation activity.

The compound provided by the present application can bind to a cereblon receptor of a CRL4^{CRBN} E3 ubiquitin ligase, so that a new binding site is generated on a protein new substrate serving as a human disease medium, thereby causing the protein degradation of the new substrate. The new morphology surface generated by these compounds can interact directly with a target protein or target protein complex, thereby directly or indirectly reducing protein levels. In various embodiments, the compound of the present application can produce a reduction in the level of a new substrate target protein through direct ubiquitination of the target protein, or ubiquitinate a new substrate target protein cofactor or target protein complex or other proteins responsible for controlling target protein homeostasis. These compounds may lead to degradation of a new substrate target protein of cerebrum that binds to a direct binding ligand, degradation of a new substrate that acts as a cofactor for the binding ligand to bind to cerebrum, degradation of cerebrum that binds to a complex cofactor and target protein surface binding ligand, degradation of a new substrate target protein complex of CRBN that binds to a binding ligand, or reduce the target protein levels by degrading the cofactor of a protein or new substrate protein not in the complex.

### Definitions

Unless otherwise stated, the following terms used in the present application shall have the following meanings. A certain term, unless otherwise specifically defined, should not be considered ambiguous or unclear, but construed according to its common meaning in the art. When referring to a trade name, it is intended to refer to its corresponding commercial product or its active ingredient.

The term “substituted” means that any one or more hydrogen atoms on a specific atom are substituted with substituents, as long as the valence of the specific atom is normal and the resulting compound is stable. When the substituent is oxo (i.e., ═O), it means that two hydrogen atoms are substituted and oxo is not possible on an aromatic group.

The term “optional” or “optionally” means that the subsequently described event or circumstance may, but does not necessarily, occur. The description includes instances where the event or circumstance occurs and instances where it does not. An “optionally substituted” group means that the group is substituted or unsubstituted. For example, ethyl being “optionally” substituted with halogen means that the ethyl may be unsubstituted (CH₂CH₃), monosubstituted (for example, CH₂CH₂F), polysubstituted (for example, CHFCH₂F, CH₂CHF₂, and the like), or fully substituted (CF₂CF₃). It will be appreciated by those skilled in the art that for any groups comprising one or more substituents, any substitutions or substituting patterns that may not exist spatially and/or cannot be synthesized are not introduced.

Cₘ₋ₙ as used herein means that the portion has an integer number of carbon atoms in the given range (m-n). For example, “C₁₋₆” means that the group may have 1 carbon atom, 2 carbon atoms, 3 carbon atoms, 4 carbon atoms, 5 carbon atoms, or 6 carbon atoms.

When any variable (e.g., R) occurs once or more in the constitution or structure of a compound, the definition of the variable in each case is independent. For example, if a group comprises 2 R, the definition of each R is independent.

When a bond is crosslinked to two atoms of a ring (including monocyclic, fused or spiro ring), the bond may be bonded to any atom on the ring (including monocyclic, fused or spiro ring). For example, the structural unit

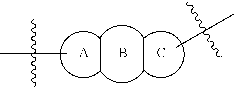

indicates that the bonds on two sides may be linked to any two different atoms on ring A, ring B, or ring C; for another example,

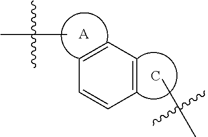

indicates that the bonds on two sides may be linked to any two different atoms on ring A, the middle benzene ring, or ring C; for further example,

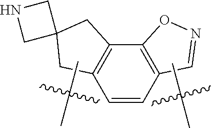

indicates that the bonds on two sides may be linked to any two different atoms of the four rings in the system.

The term “halo-” or “halogen” refers to fluorine, chlorine, bromine, and iodine.

The term “hydroxy” refers to —OH group.

The term “amino” refers to —NH₂ group.

The term “alkyl” refers to hydrocarbyl with a general formula of CₙH₂ₙ₊₁. The alkyl may be linear or branched. For example, the term “C₁₋₆ alkyl” refers to alkyl containing 1 to 6 carbon atoms (for example, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl, 2-methylpentyl, and the like). The alkyl moieties (i.e., alkyl) of alkoxy, alkylamino, dialkylamino, alkylsulfonyl, and alkylthio are similarly defined as above.

The term “alkylene” is alkyl which has lost one hydrogen.

The term “heteroalkylene” refers to alkylene in which one or more C atoms are substituted with a heteroatom and which comprises at least one C atom. Specific heteroatoms may be selected from the group consisting of N, NH, O, S, S(O), and S(O)₂. The number of the heteroatom is selected from the group consisting of 1, 2, 3, 4, 5, and 6. For example, C₁₋₁₂ heteroalkylene means that the heteroalkylene comprises 1 to 12 C atoms and one or more heteroatoms (e.g., 1-6, 1-3, 1, 2, or 3 heteroatoms). For example, C₁₋₆ heteroalkylene means that the heteroalkylene comprises 1 to 6 C atoms and one or more heteroatoms.

The term “alkoxyl” refers to —O-alkyl.

The term “alkenyl” refers to linear or branched unsaturated aliphatic hydrocarbyl consisting of carbon atoms and hydrogen atoms with at least one double bond. Non-limiting examples of alkenyl include, but are not limited to, ethenyl, 1-propenyl, 2-propenyl, 1-butenyl, isobutenyl, 1,3-butadienyl, and the like.

The term “cycloalkenyl” refers to a non-aromatic carbon ring that is not fully saturated and may exist as a monocyclic, (e.g., bicyclic) bridged cyclic or spiro cyclic structure. Unless otherwise specified, the carbon ring is usually a 4- to 12-membered ring, a 4- to 10-membered ring, or a 4- to 8-membered ring. Non-limiting examples of cycloalkenyl include, but are not limited to, cyclopentenyl, cyclopentadienyl, cyclohexenyl, cyclohexadienyl, cycloheptenyl, cycloheptadienyl, and the like.

Non-limiting examples of “benzocycloalkenyl” include benzo 4- to 12-membered cycloalkenyl, benzo 4- to 10-membered cycloalkenyl, or benzo 4- to 8-membered (e.g., 4-, 5-, 6-, 7-, or 8-membered) cycloalkenyl.

The term “cycloalkyl” refers to a carbon ring that is fully saturated and may exist as a single ring, a bridged ring, or a spiro ring. Unless otherwise specified, the carbon ring is usually a 3- to 10-membered ring (e.g., a 5- to 8-membered ring). Non-limiting examples of cycloalkyl include, but are not limited to, cyclopropyl, cyclobutyl, cyclopentyl, cyclohexyl, norbornyl(bicyclo[2.2.1]heptyl), bicyclo[2.2.2]octyl, adamantyl, and the like.

The term “heterocycloalkyl” refers to a fully saturated cyclic group that may exist in the form of a monocyclic, bridged cyclic, or spiro cyclic structure. Unless otherwise specified, the heterocyclyl is usually a 3- to 12-membered, 3- to 10-membered, 3- to 8-membered, 3- to 7-membered, 3- to 6-membered, or 3- to 5-membered ring containing 1 to 3 heteroatoms (preferably 1 or 2 heteroatoms) independently selected from the group consisting of sulfur, oxygen, and nitrogen. Examples of 3-membered heterocycloalkyl include, but are not limited to, oxiranyl, thiiranyl, and aziranyl; non-limiting examples of 4-membered heterocycloalkyl include, but are not limited to, azetidinyl, oxetanyl, and thietanyl; examples of 5-membered heterocycloalkyl include, but are not limited to, tetrahydrofuranyl, tetrahydrothienyl, pyrrolidinyl, isoxazolidinyl, oxazolidinyl, isothiazolidinyl, thiazolidinyl, imidazolidinyl, and tetrahydropyrazolyl; examples of 6-membered heterocycloalkyl include, but are not limited to, piperidinyl, tetrahydropyranyl, tetrahydrothiopyranyl, morpholinyl, piperazinyl, 1,4-oxathianyl, 1,4-dioxanyl, thiomorpholinyl, 1,3-dithianyl, and 1,4-dithianyl; examples of 7-membered heterocycloalkyl include, but are not limited to, azacycloheptanyl, oxacycloheptanyl, and thiocycloheptanyl. Monocyclic heterocycloalkyl having 5 or 6 ring atoms is preferred.

The term “heterocycloalkenyl” includes cycloalkenyl in which one or more carbon atoms are substituted with a heteroatom, such as, specifically, cycloalkenyl in which up to 3 carbon atoms, in one embodiment up to 2 carbon atoms, and in another embodiment 1 carbon atom, are each independently replaced by O, S(O), NH, or N, provided that at least one cycloalkenyl carbon-carbon double bond is preserved. A cyclic group that may exist in the form of a monocyclic, bridged cyclic, or spiro cyclic structure may be a 3- to 12-membered ring (e.g., a 5- to 8-membered ring). Examples of heterocycloalkenyl include, but are not limited to, dihydropyrrolyl, tetrahydropyridinyl, tetrahydroazepinyl, or azaspirooctene.

Non-limiting examples of “benzoheterocycloalkenyl” include benzo 4- to 12-membered heterocycloalkenyl (e.g., 5-, 6-, 10-, or 11-membered), benzo 5- to 11-membered heterocycloalkenyl, or benzo 5- to 8-membered (e.g., 4-, 5-, 6-, 7-, or 8-membered) heterocycloalkenyl. Specific examples are

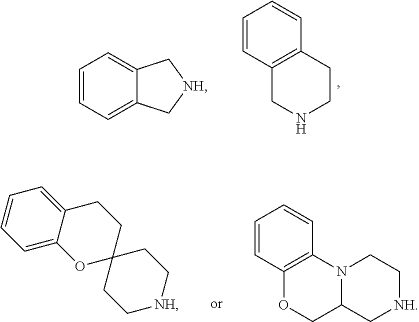 [SMILES: c1ccc2c(c1)CNC2 ; c1ccc2c(c1)CCNC2 ; "," ; c1ccc2c(c1)CCC1(CCNCC1)O2 ; "or" ; c1ccc2c(c1)OCC1CNCCN21]

Unless otherwise specified, the carbon ring is usually a 4- to 8-membered ring. Non-limiting examples of cycloalkenyl include, but are not limited to, cyclopentenyl, cyclopentadienyl, cyclohexenyl, cyclohexadienyl, cycloheptenyl, cycloheptadienyl, and the like.

The term “heteroaryl” refers to a monocyclic or fused polycyclic system which comprises at least one ring atom selected from the group consisting of N, O and S, with the remaining ring atoms being C, and which has at least one aromatic ring. Preferably, the heteroaryl has a single 4- to 8-membered ring, in particular a 5- to 8-membered ring, or has a plurality of fused rings comprising 6-14 ring atoms, in particular 6-10 ring atoms. Non-limiting examples of heteroaryl include, but are not limited to, pyrrolyl, furanyl, thienyl, imidazolyl, oxazolyl, pyrazolyl, pyridinyl, pyrimidinyl, pyrazinyl, quinolyl, isoquinolyl, tetrazolyl, triazolyl, triazinyl, benzofuranyl, benzothienyl, indolyl, isoindolyl, and the like.

In the present application one of the absolute configurations (e.g., one of [symbol] or [symbol] ; specifically

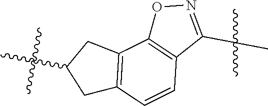

represents

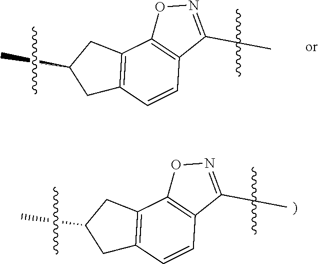

or one of the relative configurations (e.g.,

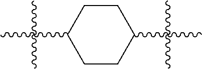

represents

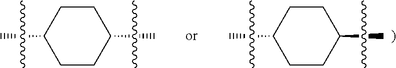

or of a stereogenic center is represented by a wavy line ( [symbol] ).

Unless otherwise specified, the compounds disclosed herein include both E and Z geometric isomers when they contain olefinic double bonds or other centers of geometric asymmetry. Likewise, all tautomeric forms are included within the scope of the present application.

The group or moiety in the present application such as LNK, Cy¹, Cy², -Cy¹-LNK-Cy²-, -Cy¹-LNK-, or -LNK-Cy²-, and specific options thereof, optionally can be linked to the left group and the right group of the group or moiety respectively in the general formula in a left-to-right reading order. For example, when Cy¹ is selected from

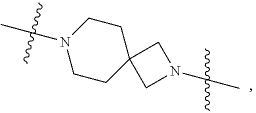

according to a left-to-right reading order, the left side of Cy¹ is linked to the structural moiety

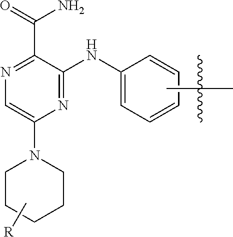

in the general formula corresponding to the left side, and the right side is linked to the right moiety

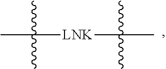

forming the structural moiety

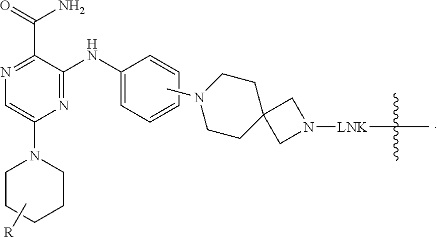

Optionally, the group or moiety in the present application such as LNK, Cy¹, Cy², -Cy¹-LNK-Cy²-, -Cy¹-LNK-, or -LNK-Cy²-, and specific options thereof, can be linked to the left group and the right group of the group or moiety respectively in the general formula in a right-to-left reading order. For example, when Cy¹ is selected from

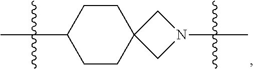

according to a right-to-left reading order, the right side of Cy¹ is linked to the structural moiety

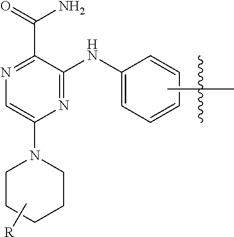

in the general formula corresponding to the left side, and the left side of Cy¹ is linked to the structural moiety

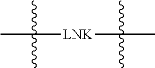

in the general formula corresponding to the right side to form the structural moiety

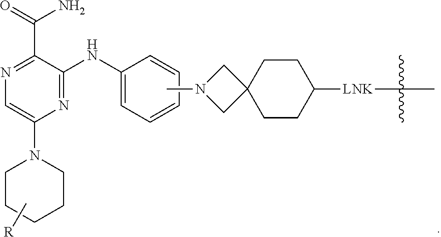

The other groups are as described above.

The term “proteolysis targeting chimera (Protac) molecule” is a bifunctional compound capable of combining a target protein and E3 ubiquitin ligase simultaneously. Such compounds can induce the target protein to be recognized by proteasomes of cells, cause the degradation of the target protein and effectively reduce the content of the target protein in the cells.

The term “derivative” refers to one or a group of new compounds produced by substituting or replacing one or more hydrogen atoms in the basic structure of the parent compound with other groups or structural moieties. The derivative of the present application refers to a derivative compound that retains the parent structure. For example, the parent compound is derived into a Protac molecule, in particular a PTM-linker-ULM molecule, wherein PTM is a protein target moiety binding to the target protein and target polypeptide; the linker is a connecting group, and ULM refers to a moiety binding to a ubiquitin ligase.

The term “protein-targeting drug or derivative thereof” or PTM is used to describe small molecules that bind to a target protein or other proteins or polypeptides of interest and bring the localization/presence of the protein or polypeptide close to the ubiquitin ligase so that degradation of the protein or polypeptide by the ubiquitin ligase can occur. Non-limiting examples of small molecule target protein binding moieties include drugs or derivatives thereof, among numerous others, that target AR, ER, kinase, phosphatase, MDM2, human BET bromodomain protein, Hsp90, HDAC, human lysine methyltransferase, RAF receptor, FKBP, angiogenic factor, immunosuppression-related receptor or protein, arene receptor, thyroid hormone receptor, HIV protease, HIV integrase, HCV protease, HBV protease, or acyl protein thioesterase 1 and/or 2, or include drugs or derivatives thereof, among numerous others, that target ALK, BET, CDK, PARP, EGFR, γ-secretase, CBFβ-SMMHC, WEE1, MEK, BCR-ABL, MET, RAS, BTK, VEGFR, JAK, HER2, HDAC, Akt, PI3K, mTOR, AR, ER, PDEB, SRC, MDM2, RAF, IRAK4, STAT3, and c-Myc, or include drugs or derivatives thereof, among numerous others, that target ALK, BRD4, CDK4/6, PARP, EGFR, γ-secretase, CBFβ-SMMHC, WEE1, MEK, BCR-ABL, MET, KRAS, EGFR, BTK, AR, ER, PDEB, JAK, MDM2, or RAF.

The term “PTM group” is a drug or a derivative thereof that binds to a target protein. There are many types of targets of PTM groups, and the PTM groups are selected from the group consisting of proteins that are expressed in a cell such that at least a portion of the sequence is found in the cell, and the protein can bind to the PTM group. The term “protein” includes oligopeptide and polypeptide sequences with sufficient length that can bind to the PTM group according to the present application. Any protein in eukaryotic systems or microbial systems (including viruses, bacteria, or fungi) as further described herein is a target for ubiquitination mediated by the compounds according to the present application. The target protein is preferably a eukaryotic protein.

“Target proteins” are used to describe below as proteins or polypeptides that bind to the compounds according to the present application and are targets for degradation by ubiquitin ligases. Such small molecule target protein binding moieties also include pharmaceutically acceptable salts, enantiomers, solvates, and polymorphs of the compositions, and other small molecules that can target the protein of interest. The binding moieties are linked to the group

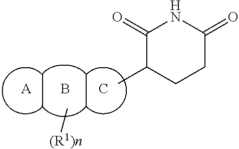

via a linker group L.

Unless otherwise specified,

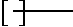

is used to indicate that a hydrogen atom at any position of a group within [ ] may be substituted with a group linked by “-”.

The term “treat” or “treatment” means administering a compound or formulation described herein to ameliorate or eliminate a disease or one or more symptoms associated with the disease, including:
(i) inhibiting a disease or disease state, i.e., arresting its progression; and
(ii) alleviating a disease or disease state, i.e., causing its regression.

The term “prevent”, “preventing”, or “prevention” means administering the compound or formulation of the present application to prevent a disease or one or more symptoms associated with the disease, and includes: preventing the occurrence of the disease or disease state in a mammal, particularly when such a mammal is predisposed to the disease state but has not yet been diagnosed with it.

The term “therapeutically effective amount” refers to an amount of the compound disclosed herein for (i) treating or preventing a specific disease, condition, or disorder; (ii) alleviating, ameliorating, or eliminating one or more symptoms of a specific disease, condition or disorder, or (iii) preventing or delaying onset of one or more symptoms of a specific disease, condition, or disorder described herein. The amount of the compound disclosed herein composing the “therapeutically effective amount” varies depending on the compound, the disease state and its severity, the administration regimen, and the age of the mammal to be treated, but can be determined routinely by those skilled in the art in accordance with their knowledge and the present disclosure.

The term “pharmaceutically acceptable” is used herein for those compounds, materials, compositions, and/or dosage forms that are, within the scope of sound medical judgment, suitable for use in contact with the tissues of human beings and animals without excessive toxicity, irritation, allergic response, or other problems or complications, and commensurate with a reasonable benefit/risk ratio.

A pharmaceutically acceptable salt, for example, may be a metal salt, an ammonium salt, a salt formed with an organic base, a salt formed with an inorganic acid, a salt formed with an organic acid, a salt formed with a basic or acidic amino acid, and the like.

The term “pharmaceutical composition” refers to a mixture consisting of one or more of the compounds or the salts thereof of the present application and a pharmaceutically acceptable excipient. The pharmaceutical composition is intended to facilitate the administration of the compound of the present application to an organism.

The term “pharmaceutically acceptable excipient” refers to those that do not have a significant irritating effect on an organism and do not impair the biological activity and properties of the active compound. Suitable excipients are well known to those skilled in the art, for example, carbohydrate, wax, water-soluble and/or water-swellable polymers, hydrophilic or hydrophobic material, gelatin, oil, solvent, or water.

The word “comprise” and variations thereof such as “comprises” or “comprising” will be understood in an open, non-exclusive sense, i.e., “including but not limited to”.

The compounds and intermediates disclosed herein may also exist in different tautomeric forms, and all such forms are included within the scope of the present application. The term “tautomer” or “tautomeric form” refers to structural isomers of different energies that can interconvert via a low energy barrier. For example, a proton tautomer (also referred to as prototropic tautomer) includes interconversion via proton transfer, such as keto-enol isomerism and imine-enamine isomerization. A specific example of a proton tautomer is an imidazole moiety where a proton can transfer between two ring nitrogens. A valence tautomer includes the interconversion via recombination of some bonding electrons.

Unless otherwise specified clearly herein, singular terms encompass plural terms, and vice versa. Similarly, unless otherwise specified clearly herein, the word “or” is intended to include “and”.

Unless otherwise stated herein, parameter values representing amounts of ingredients or physicochemical properties or reaction conditions and the like are to be understood as being modified in all cases by the term “about”. When the term “about” is used to describe the present application, the term “about” indicates that there is an error value; for example, it means varying within a range of ±5%, such as ±1% or ±0.1%, of a particular value.

The present application also comprises isotopically labeled compounds of the present application which are identical to those recited herein but have one or more atoms replaced by an atom having an atomic mass or mass number different from the atomic mass or mass number generally found in nature. Examples of isotopes that can be incorporated into the compounds of the present application include isotopes of hydrogen, carbon, nitrogen, oxygen, phosphorus, sulfur, fluorine, iodine and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I, and ³⁶Cl.

Certain isotopically labeled compounds disclosed herein (e.g., those labeled with ³H and ¹⁴C) can be used to analyze compounds and/or substrate tissue distribution. Tritiated (i.e., ³H) and carbon-14 (i.e., ¹⁴C) isotopes are particularly preferred for their ease of preparation and detectability. Positron emitting isotopes, such as ¹⁵O, ¹³N, ¹¹C, and ¹⁸F, can be used in positron emission tomography (PET) studies to determine substrate occupancy. Isotopically labeled compounds of the present application can generally be prepared by following procedures analogous to those disclosed in the schemes and/or examples below while substituting a non-isotopically labeled reagent with an isotopically labeled reagent.

Furthermore, substitution with heavier isotopes such as deuterium (i.e., ²H) may provide certain therapeutic advantages (e.g., increased in vivo half-life or reduced dosage requirement) resulting from greater metabolic stability and hence may be preferred in some circumstances in which deuterium substitution may be partial or complete, wherein partial deuterium substitution refers to substitution of at least one hydrogen with at least one deuterium.

The compound disclosed herein can be asymmetrical, for example, having one or more stereoisomers. Unless otherwise stated, all stereoisomers include, for example, enantiomers and diastereoisomers. The compound with asymmetrical carbon atoms disclosed herein can be separated in an optically pure form or in a racemic form. The optically pure form can be separated from a racemic mixture or can be synthesized using a chiral raw material or a chiral reagent.

The pharmaceutical composition of the present application can be prepared by combining the compound of the present application with a suitable pharmaceutically acceptable excipient, and can be formulated, for example, into a solid, semisolid, liquid or gaseous formulation such as tablet, pill, capsule, powder, granule, ointment, emulsion, suspension, suppository, injection, inhalant, gel, microsphere, and aerosol.

Typical routes of administration of the compound or the pharmaceutically acceptable salt thereof or the pharmaceutical composition thereof of the present application include, but are not limited to, oral, rectal, local, inhalation, parenteral, sublingual, intravaginal, intranasal, intraocular, intraperitoneal, intramuscular, subcutaneous and intravenous administration.

The pharmaceutical composition of the present application can be manufactured using methods well known in the art, such as conventional mixing, dissolving, granulating, dragee-making, levigating, emulsifying and lyophilizing.

In some embodiments, the pharmaceutical composition is in an oral form. For oral administration, the pharmaceutical composition can be formulated by mixing the active compounds with pharmaceutically acceptable excipients well known in the art. These excipients enable the compounds of the present application to be formulated into tablets, pills, pastilles, dragees, capsules, liquids, gels, slurries, suspensions, and the like for oral administration to a patient.

A solid oral composition can be prepared by conventional mixing, filling or tableting. For example, it can be obtained by the following method: mixing the active compounds with solid excipients, optionally grinding the resulting mixture, adding additional suitable excipients if desired, and processing the mixture into granules to get the core parts of tablets or dragees. Suitable excipients include, but are not limited to: binders, diluents, disintegrants, lubricants, glidants, sweeteners or flavoring agents, and the like.

The pharmaceutical composition may also be suitable for parenteral administration, such as a sterile solution, a suspension, or a lyophilized product in a suitable unit dosage form.

In all of the administration methods of the compound of general formula I described herein, the daily dose administered is from 0.01 to 200 mg/kg body weight, given in individual or separated doses.

The compounds disclosed herein can be prepared using a variety of synthetic methods well known to those skilled in the art, including the specific embodiments listed below, embodiments formed by combinations thereof with other chemical synthetic methods, and equivalents thereof known to those skilled in the art. The preferred embodiments include, but are not limited to, the examples of the present application.

The chemical reactions of the embodiments of the present application are carried out in a proper solvent that should be suitable for the chemical changes in the present application and the reagents and materials required. In order to obtain the compounds of the present application, it is sometimes necessary for those skilled in the art to modify or select a synthesis procedure or a reaction process based on the existing embodiments.

An important consideration in synthetic route planning in the art is the selection of suitable protecting groups for reactive functional groups (e.g., amino in the present application). For example, reference may be made to Greene's Protective Groups in Organic Synthesis (4th Ed.) Hoboken, New Jersey: John Wiley & Sons, Inc.

In some embodiments, the compound of formula I disclosed herein can be prepared by one skilled in the art of organic synthesis through the following routes:

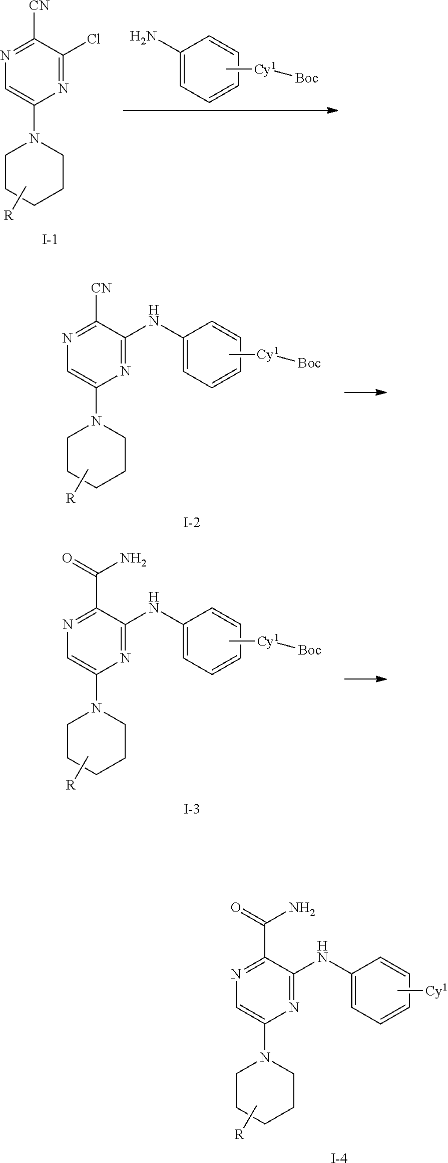

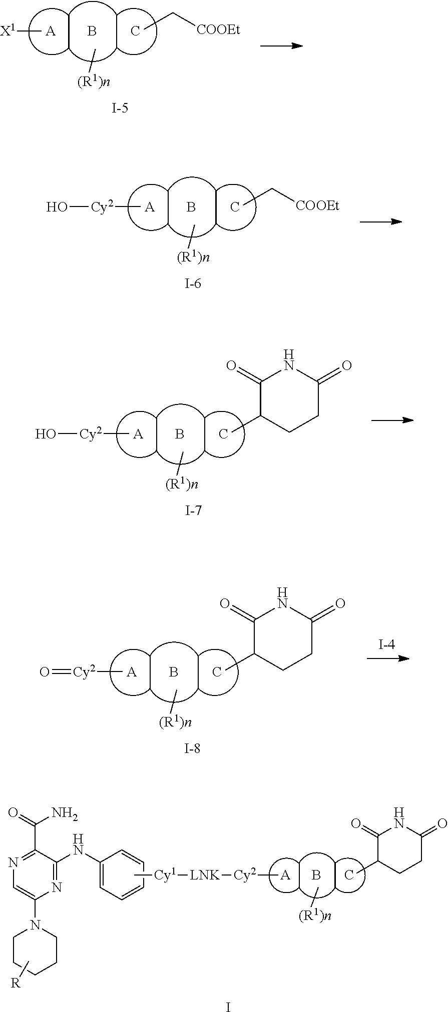

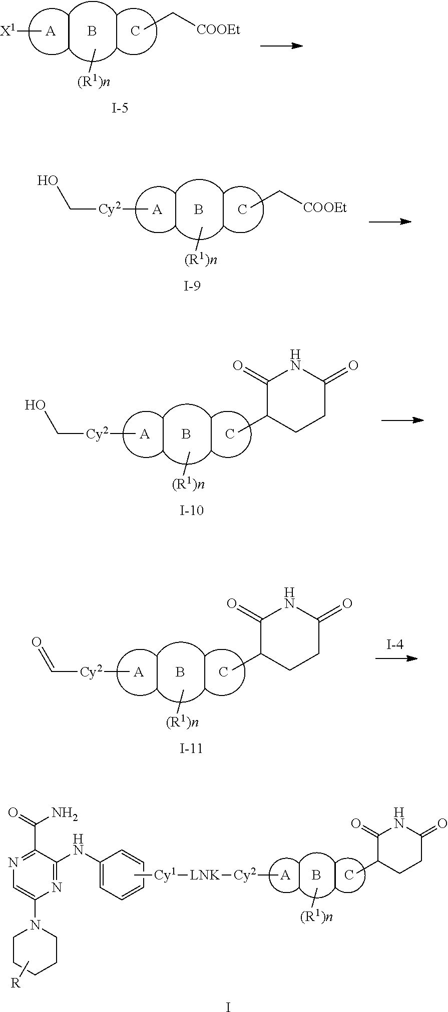

wherein X¹ is selected from the group consisting of halogens.

A compound of general formula (I-1) is subjected to a coupling reaction to give a compound of general formula (I-2), the compound of general formula (I-2) is subjected to a hydrolysis reaction to give a compound of general formula (I-3), and then a protecting group is removed to give a compound of general formula (I-4).

A compound of general formula (I-5) is subjected to a coupling reaction with hydroxy-substituted Cy² to give a compound of general formula (I-6), and the compound of general formula (I-6) is subjected to an addition reaction with acrylamide and then an amine-ester exchange reaction to give a compound of general formula (I-7). The compound of general formula (I-7) is subjected to an oxidation reaction to give a compound of general formula (I-8). The compound of general formula (I-4) and the compound of general formula (I-8) are subjected to a reductive amination reaction to give a compound of general formula I.

The compound of general formula (I-5) is subjected to a coupling reaction with hydroxymethyl-substituted Cy² to give a compound of general formula (I-9), and the compound of general formula (I-9) is subjected to an addition reaction with acrylamide and then an amine-ester exchange reaction to give a compound of general formula (I-10). The compound of general formula (I-10) is subjected to an oxidation reaction to give a compound of general formula (I-11). The compound of general formula (I-4) and the compound of general formula (I-11) are subjected to a reductive amination reaction to give a compound of general formula I.

The following abbreviations are used in this application:

Boc represents tert-butyloxycarbonyl; Et represents ethyl; EA represents ethyl acetate; DMSO represents dimethyl sulfoxide; DMF represents N,N-dimethylformamide; BINAP represents 2,2′-bis(diphenylphosphino)-1,1′-binaphthyl; DCM represents dichloromethane; Pd₂(dba)₃ represents tris(dibenzylideneacetone)dipalladium; THE represents tetrahydrofuran; MeOH represents methanol; PE represents petroleum ether; IBX represents 2-iodoxybenzoic acid; DIPEA represents N,N′-diisopropylethylamine; DIBAL-H represents diisobutylaluminum hydride; NIS represents N-iodosuccinimide; NBS represents N-bromosuccinimide; Tf represents —OSO₂CF₃; H₂O₂ represents hydrogen peroxide; Pd(OAc)₂ represents palladium acetate; DMA represents N,N-dimethylacetamide; TBSCl represents tert-butyldimethylsilyl chloride; TFA represents trifluoroacetic acid; PdCl₂(dppf) represents [1,1′-bis(diphenylphosphino)ferrocene]palladium dichloride; Ruphos represents 2-dicyclohexylphosphino-2′,6′-diisopropoxybiphenyl.

All patents, patent applications and other identified publications are explicitly incorporated herein by reference for the purpose of description and disclosure. These publications are provided solely because they were disclosed prior to the filing date of the present application. All statements as to the dates of these documents or descriptions as to the contents of these documents are based on the information available to the applicant and do not constitute any admission as to the correctness of the dates or the content of these documents. Moreover, in any country or region, any reference to these publications herein is not to be construed as an admission that the publications form part of the commonly recognized knowledge in the art.

For clarity, the present application is further described with the following examples, which are, however, not intended to limit the scope of the present application. All reagents used in the present application are commercially available and can be used without further purification.

### DETAILED DESCRIPTION

### Example 1: Synthesis of Compound 1

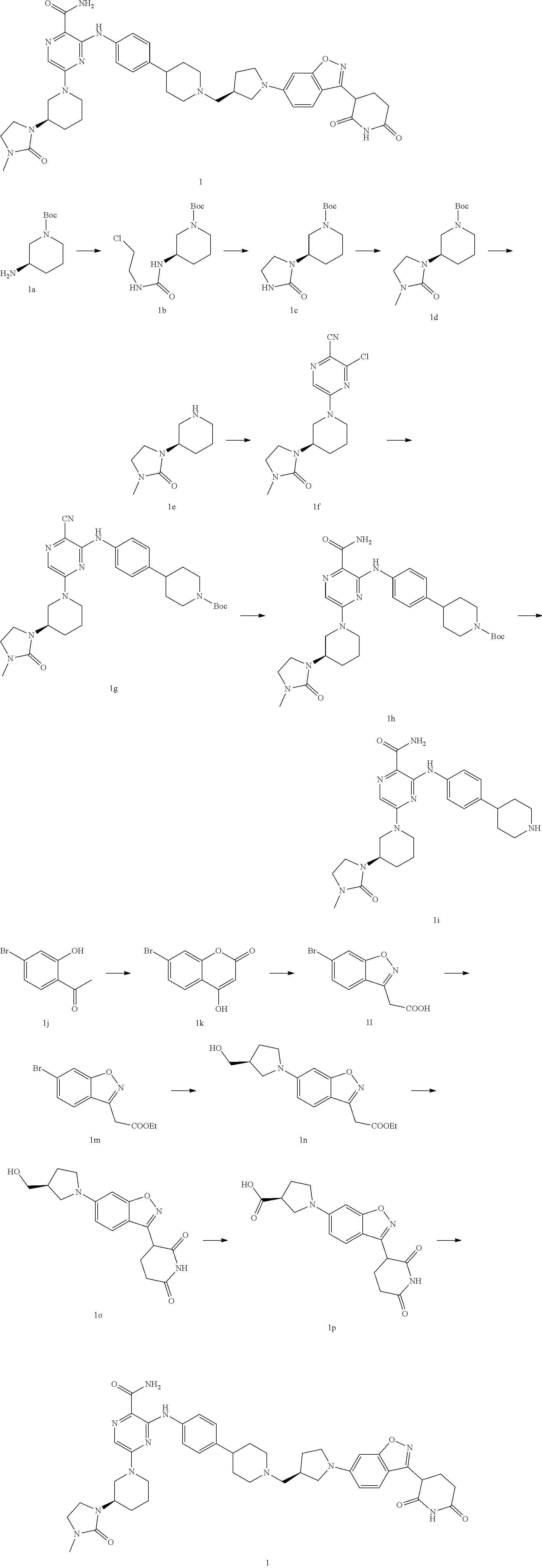

### Step 1: Preparation of Intermediate 1b

Intermediate 1a (25 g), triethylamine (25.3 g, 34.8 mL), and dichloromethane (250 mL) were added to a reaction flask in sequence. 1-Chloro-2-isocyanate (15.81 g) was slowly added dropwise, and after the dropwise addition was carried out for 20 min and completed, the mixture was reacted at room temperature for 4 h. 500 mL of water was added to the reaction system, the organic phase was separated, and the aqueous phase was extracted 2 times with 200 mL of dichloromethane. The organic phases were combined, dried over sodium sulfate, and filtered. The filtrate was distilled under reduced pressure to remove the solvent, and the crude product was separated by silica gel column chromatography (eluent: EA) to give the target intermediate 1b (40.8 g).

¹H NMR (500 MHz, DMSO-d₆) δ 6.11 (q, J=7.1, 6.5 Hz, 2H), 3.56 (s, 1H), 3.43 (dq, J=8.6, 4.5 Hz, 1H), 3.33-3.27 (m, 2H), 3.14-2.84 (m, 1H), 1.80-1.71 (m, 1H), 1.60 (ddq, J=12.2, 6.0, 3.5, 3.0 Hz, 1H), 1.38 (s, 9H), 1.37-1.24 (m, 2H).

### Step 2: Preparation of Intermediate 1c

Sodium hydride (10.31 g) was slowly added to a stirred solution of intermediate 1b (40 g) in tetrahydrofuran (300 mL) at 0° C. After the addition was completed, the mixture was reacted overnight at room temperature. After the reaction was completed, 75 mL of water was added to the reaction solution to quench the reaction. After the mixture was vigorously stirred for 10 min, liquid separation was performed. The aqueous phase was extracted 3 times with 200 mL of dichloromethane, dried over anhydrous sodium sulfate, and concentrated by rotary evaporation. The concentrate was extracted with 300 mL of acetonitrile and 300 mL of petroleum ether, and liquid separation was performed. The acetonitrile phase was subjected to rotary evaporation to give intermediate 1c (32.2 g).

### Step 3: Preparation of Intermediate 1d

Sodium hydride (5.71 g) was slowly added to a stirred solution of intermediate 1c (20 g) in tetrahydrofuran (200 mL) at 0° C. After the mixture was stirred for 5 min, ice bath was removed. After the mixture was stirred at room temperature for 1 h, ice bath was added. Iodomethane (15.21 g, 6.70 mL) was slowly added dropwise to the reaction system, and after the dropwise addition was completed, the mixture was reacted overnight at room temperature. After the reaction was completed, 75 mL of water was added to the reaction solution to quench the reaction. After the mixture was vigorously stirred for 10 min, liquid separation was performed. The aqueous phase was extracted 3 times with 200 mL of dichloromethane, dried over anhydrous sodium sulfate, and concentrated by rotary evaporation. The concentrate was extracted with 300 mL of acetonitrile and 300 mL of petroleum ether, and liquid separation was performed. The acetonitrile phase was subjected to rotary evaporation to give intermediate 1d (22.5 g).

### Step 4: Preparation of Intermediate 1e

Intermediate 1d (20 g) and a solution of 4 M hydrochloric acid in dioxane (25.7 g, 176 mL, 705 mmol) were added to a single-necked flask in sequence, and the mixture was reacted at room temperature. After the reaction was completed, the solvent was removed by evaporation at reduced pressure to give intermediate 1e (15.5 g).

### Step 5: Preparation of Intermediate 1f

3,5-Dichloropyrazine-2-carbonitrile (11.82 g) was added to a stirred solution of intermediate 1e (15 g) and N,N-diisopropylethylamine (35.1 g, 47.5 mL) in DMF (300 mL) at 0° C. Ice bath was removed after 15 min, and the mixture was stirred at room temperature overnight. 300 mL of ethyl acetate and 300 mL of water were added to the reaction solution. The organic phase was separated, extracted 2 times with 100 mL of ethyl acetate, washed with 200 mL of saturated brine, dried over anhydrous sodium sulfate, filtered, and concentrated by evaporation at reduced pressure to remove the solvent, and the crude product was separated by silica gel column chromatography (eluent: dichloromethane/methanol=100:1, v/v) to give the target intermediate 1f (14.5 g).

MS(ESI, [M+H]⁺) m/z: 321.2.

¹H NMR (500 MHz, DMSO-d₆) δ 7.94 (s, 1H), 3.30 (dtd, J=14.7, 7.5, 6.8, 4.9 Hz, 2H), 3.25-3.22 (m, 1H), 2.89 (s, 4H), 2.73 (s, 3H), 2.65 (s, 2H), 1.92-1.69 (m, 3H), 1.62-1.48 (m, 1H).

### Step 6: Preparation of Intermediate 1g

Intermediate 1f (13 g), tert-butyl 4-(4-aminophenyl)piperidine-1-carboxylate (9.61 g), cesium carbonate (34.0 g), BINAP (2.166 g), palladium acetate (0.781 g), and 1,4-dioxane (200 mL) were added to a single-necked flask in sequence, and the mixture was heated to 100° C. and reacted under N₂ atmosphere. After the reaction was completed, the reaction solution was cooled to room temperature and filtered, and the filter cake was washed with 150 mL of dichloromethane. The filtrate was concentrated by evaporation at reduced pressure to remove the solvent, and the crude product was separated by silica gel column chromatography (eluent: dichloromethane/methanol=100:1, v/v) to give the target intermediate 1g (14.2 g).

MS(ESI, [M+H]⁺) m/z: 561.5

¹H NMR (500 MHz, DMSO-d₆) δ 8.97 (s, 1H), 7.82 (s, 1H), 7.48-7.43 (m, 2H), 7.16-7.10 (m, 2H), 4.37-4.19 (m, 2H), 4.07 (d, J=13.1 Hz, 2H), 3.64-3.59 (m, 2H), 3.42 (dq, J=11.6, 3.0 Hz, 3H), 3.34-3.29 (m, 1H), 3.26-3.23 (m, 2H), 2.94 (d, J=12.9 Hz, 1H), 2.71 (s, 3H), 2.62 (ddt, J=15.5, 12.0, 3.3 Hz, 1H), 1.82-1.71 (m, 5H), 1.58-1.43 (m, 3H), 1.42 (s, 9H).

### Step 7: Preparation of Intermediate 1h

Intermediate 1g (8 g), DMSO (25 mL), and cesium carbonate (4.08 g) were added to a single-necked flask in sequence. Hydrogen peroxide (21.32 g, 19.21 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent, and the crude product was separated by silica gel column chromatography (eluent: dichloromethane/methanol=100:1, v/v) to give the target intermediate 1b (7.22 g).

MS(ESI, [M+H]⁺) m/z: 579.51.

¹H NMR (500 MHz, DMSO-d₆) δ11.19 (s, 1H), 7.75 (d, J=2.8 Hz, 1H), 7.66 (s, 1H), 7.55-7.46 (m, 2H), 7.32 (d, J=2.8 Hz, 1H), 7.15 (d, J=8.6 Hz, 2H), 4.36 (d, J=12.4 Hz, 1H), 4.27 (d, J=13.4 Hz, 1H), 4.06 (d, J=12.8 Hz, 2H), 3.61 (tt, J=11.0, 4.0 Hz, 1H), 3.25 (dd, J=9.5, 7.2 Hz, 2H), 3.06-2.91 (m, 2H), 2.70 (s, 3H), 2.62 (tt, J=12.1, 3.6 Hz, 1H), 2.54 (s, 4H), 1.82 (dt, J=14.8, 3.5 Hz, 2H), 1.79-1.70 (m, 3H), 1.54 (s, 1H), 1.45 (dq, J=12.9, 4.2 Hz, 2H), 1.42 (s, 9H).

### Step 8: Preparation of Intermediate 1i

Intermediate 1h (3 g), dichloromethane (30 mL), and trifluoroacetic acid (17.06 g, 11.53 mL) were added to a single-necked flask in sequence, and the mixture was reacted at room temperature. After the reaction was completed, the solvent was removed by evaporation at reduced pressure to give a product of trifluoroacetate salt. 50 mL of dichloromethane was added to dissolve the crude product, and a saturated aqueous sodium bicarbonate solution was slowly added. After the system was adjusted to weak alkalinity, liquid separation was performed. The organic phase was concentrated by evaporation at reduced pressure to remove the solvent, thus giving intermediate 1i (2.4 g).

MS(ESI, [M+H]⁺) m/z: 479.44

¹H NMR (500 MHz, DMSO-d₆) δ 11.25 (s, 1H), 7.77 (d, J=2.8 Hz, 1H), 7.67 (s, 1H), 7.54 (d, J=8.3 Hz, 2H), 7.34 (d, J=2.8 Hz, 1H), 7.19-7.12 (m, 2H), 4.32 (dd, J=33.8, 12.7 Hz, 2H), 3.62 (tt, J=11.0, 4.0 Hz, 2H), 3.39-3.31 (m, 3H), 3.30-3.23 (m, 3H), 3.05 (t, J=11.7 Hz, 1H), 3.00-2.88 (m, 3H), 2.72 (s, 3H), 1.95-1.86 (m, 2H), 1.85-1.79 (m, 2H), 1.79-1.64 (m, 3H), 1.57 (dtd, J=16.9, 8.4, 3.7 Hz, 1H).

### Step 9: Preparation of Intermediate 1k

A solution of intermediate 1j (30 g) in toluene (100 mL) was slowly added dropwise to a solution of sodium hydride (27.9 g) in toluene (50 mL) at 0° C. After the dropwise addition was completed, the mixture was stirred at 0° C. for 15 min. Dimethyl carbonate (126 g) was added dropwise to the reaction solution. After the dropwise addition was completed and the mixture was stirred at 0° C. for 15 min, the mixture was heated to 100° C. and reacted for 2 h. The reaction solution was washed once with water (500 mL). The organic layer was discarded, and the aqueous layer was adjusted to pH=2 to 3 with diluted hydrochloric acid (3 mol/L) at a low temperature, and a large amount of solid was precipitated. After filtration under vacuum and drying, 30.63 g of intermediate 1k was obtained.

MS (ESI, [M−H]⁻) m/z: 239.0

¹H NMR (500 MHz, DMSO-d₆) δ 12.74 (s, 1H), 7.77-7.65 (m, 2H), 7.54 (dd, J=8.4, 1.9 Hz, 1H), 5.63 (s, 1H).

### Step 10: Preparation of Intermediate 1l

Intermediate 1k (29.5 g), hydroxylamine hydrochloride (15.85 g), sodium ethoxide (15.52 g), and ethanol (150 mL) were added to a reaction flask in sequence, and the mixture was heated to 85° C. for 2 h under N₂ atmosphere. The reaction solution was subjected to rotary evaporation to dryness, 500 mL of a saturated sodium carbonate solution was added to the residue, and the mixture was extracted with DCM (300 mL×2). The organic layer was discarded. The pH of the aqueous layer was adjusted to <3 with diluted hydrochloric acid (3 mol/L). A large amount of solid was precipitated. Filter under vacuum was performed, and the resulting filter cake was washed with water and dried under atmospheric pressure to give 26.53 g of intermediate 1l.

MS (ESI, [M−H]⁻) m/z: 254.0.

¹H NMR (500 MHz, DMSO-d₆) δ 12.95 (s, 1H), 8.12 (d, J=1.5 Hz, 1H), 7.82 (d, J=8.4 Hz, 1H), 7.59 (dd, J=8.4, 1.6 Hz, 1H), 4.12 (s, 2H).

### Step 11: Preparation of Intermediate 1m

Intermediate 1l (26.5 g), and ethanol (530 mL) were added to a reaction flask in sequence. After concentrated sulfuric acid (49.9 g, 27.1 mL, 508 mmol) was slowly added dropwise, the mixture was heated to 85° C. and reacted for 4 h. The reaction solution was subjected to rotary evaporation to dryness. After ice water was added to the residue, an NaOH solution was added dropwise at a low temperature to adjust the pH to be approximately equal to 9, and then the mixture was extracted 2 times with 300 mL of ethyl acetate. The organic layers were combined, dried over anhydrous sodium sulfate, and filtered under vacuum. The filtrate was subjected to rotary evaporation to dryness to give 24.87 g of intermediate 1m.

MS (ESI, [M−H]⁻) m/z: 282.0

¹H NMR (500 MHz, DMSO-d₆) δ 8.13 (d, J=1.5 Hz, 1H), 7.82 (d, J=8.4 Hz, 1H), 7.60 (dd, J=8.4, 1.5 Hz, 1H), 4.23 (s, 2H), 4.15 (q, J=7.1 Hz, 2H), 1.19 (t, J=7.1 Hz, 3H).

### Step 12: Preparation of Intermediate 1n

Intermediate 1m (2 g) and (S)-pyrolidine-3-methanol (2.087 g) were added in sequence to a reaction flask. Pd₂(dba)₃ (1.260 g) and potassium phosphate (5.84 g) were then added, and finally toluene (60 mL) was added. The mixture was heated at 80° C. for 4 h under N₂ atmosphere. The reaction solution was washed twice with 100 mL of water. The organic layer was dried over anhydrous sodium sulfate and filtered under vacuum, and the resulting filtrate was concentrated, separated and purified by silica gel column chromatography to give intermediate 1n (0.35 g).

MS (ESI, [M+H]⁺) m/z: 305.20.

### Step 13: Preparation of Intermediate 1o

Intermediate 1n (360 mg) and THF (30 mL) were first added to a reaction flask, acrylamide (101 mg) and potassium tert-butoxide (199 mg) were added at 0° C., and the mixture was slowly warmed to 10° C. under N₂ atmosphere and stirred. After the reaction was completed, 100 mL of a saturated ammonium chloride solution was added to the reaction solution at a low temperature, and the mixture was extracted twice with EA (50 mL). The organic layer was dried over anhydrous sodium sulfate and filtered, the filtrate was subjected to rotary evaporation to dryness and purified by silica gel column chromatography (eluent: DCM:MeOH=95:5, v/v) to give 245 mg of intermediate 1o.

MS (ESI, [M+H]⁺) m/z: 330.1

### Step 14: Preparation of Intermediate 1p

Intermediate 1o (330 mg) and DCM (30 mL) were added to a reaction flask in sequence, Dess-Martin reagent (1.224 g) was added with stirring, and the mixture was reacted at room temperature under N₂ atmosphere. After the reaction was completed, an aqueous Na₂S2O₃ solution (30 mL) was added at a low temperature to quench the reaction solution, and the mixture was extracted with an aqueous NaHCO₃ solution (30 mL). The organic layer was dried over anhydrous sodium sulfate and filtered under vacuum, and the resulting filtrate was subjected to rotary evaporation to dryness to give 290 mg of intermediate 1p, which was directly used in the next step.

### Step 15: Preparation of Compound 1

Intermediate 1l (150 mg), intermediate 1p (103 mg), DCM (10 mL), and glacial acetic acid (31.4 mg, 0.030 mL) were added to a reaction flask in sequence. After the mixture was stirred at room temperature for 20 min, sodium cyanoborohydride (59.1 mg) was added, and the mixture was stirred at room temperature. After the reaction was completed, 20 mL of a saturated NaHCO₃ solution was added to the reaction solution, and the mixture was extracted twice with 20 mL of DCM-MeOH (10:1). The extracts were combined, dried over anhydrous sodium sulfate, and filtered under vacuum, and the resulting filtrate was subjected to rotary evaporation to dryness and purified by C₁₈ reversed-phase column (10 nM aqueous ammonium acetate solution-acetonitrile=50%:50%, v/v) to give 55 mg of compound 1.

Q-TOF (ESI, [M+H]⁺) m/z: 790.4146

¹H NMR (500 MHz, DMSO-d₆) δ 11.18 (s, 1H), 11.02 (s, 1H), 7.75 (d, J=2.8 Hz, 1H), 7.66 (s, 1H), 7.52 (dd, J=13.9, 8.4 Hz, 3H), 7.35-7.30 (m, 1H), 7.17 (d, J=8.1 Hz, 2H), 6.66 (dd, J=8.9, 1.8 Hz, 1H), 6.58 (d, J=1.8 Hz, 1H), 4.45-4.34 (m, 2H), 4.28 (d, J=13.3 Hz, 1H), 3.62 (dt, J=11.1, 7.0 Hz, 1H), 3.48 (t, J=8.5 Hz, 1H), 3.41 (d, J=4.7 Hz, 1H), 3.26 (dd, J=10.3, 7.0 Hz, 3H), 3.12-2.92 (m, 5H), 2.73 (s, 4H), 2.60 (ddd, J=21.9, 12.8, 5.5 Hz, 2H), 2.41 (ddd, J=25.0, 11.8, 4.7 Hz, 4H), 2.20-2.10 (m, 2H), 1.87-1.51 (m, 10H).

### Example 2: Synthesis of Compound 2

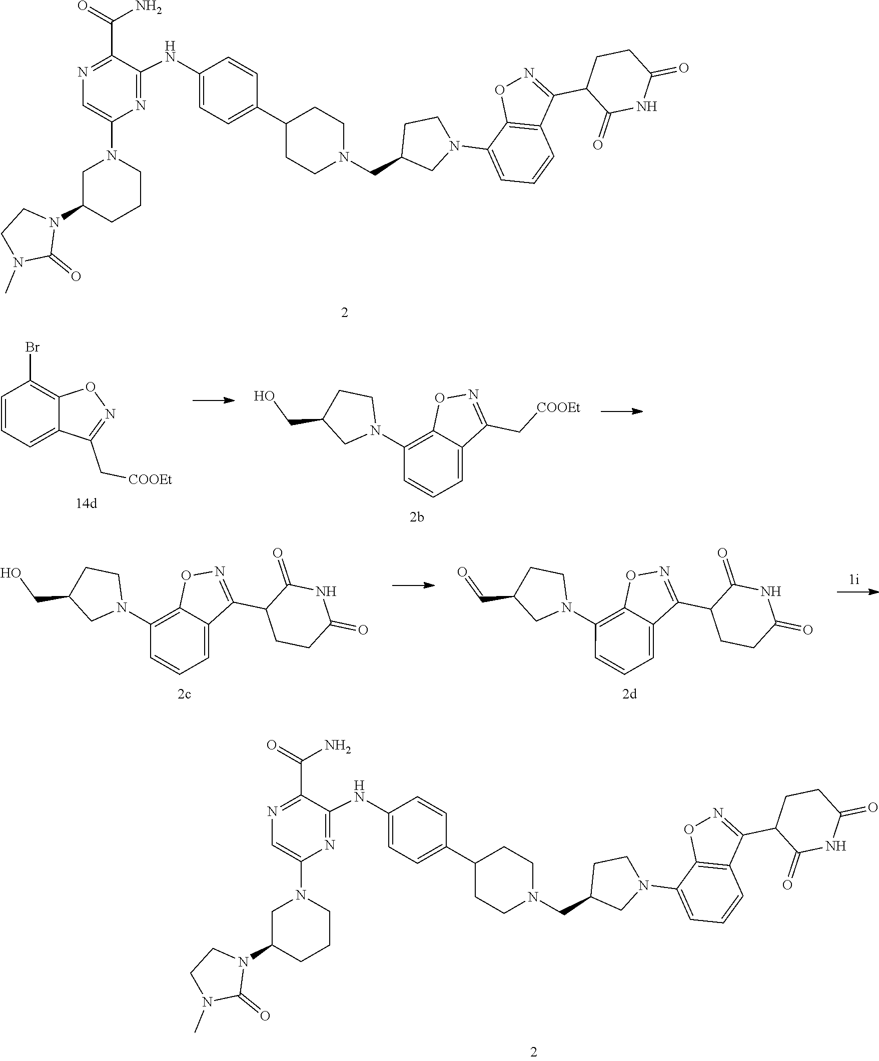 [SMILES: CN1CCN([C@@H]2CCCN(c3cnc(C(N)=O)c(Nc4ccc(C5CCN(C[C@H]6CCN(c7cccc8c(C9CCC(=O)NC9=O)noc78)C6)CC5)cc4)n3)C2)C1=O ; CCOC(=O)Cc1noc2c(Br)cccc12 ; CCOC(=O)Cc1noc2c(N3CC[C@H](CO)C3)cccc12 ; O=C1CCC(c2noc3c(N4CC[C@H](CO)C4)cccc23)C(=O)N1 ; O=C[C@H]1CCN(c2cccc3c(C4CCC(=O)NC4=O)noc23)C1 ; CN1CCN([C@@H]2CCCN(c3cnc(C(N)=O)c(Nc4ccc(C5CCN(C[C@H]6CCN(c7cccc8c(C9CCC(=O)NC9=O)noc78)C6)CC5)cc4)n3)C2)C1=O]

### Step 1: Preparation of Intermediate 2b

Intermediate 14d (2 g) and (S)-pyrrolidine-3-methanol (1.424 g) were added in sequence to a reaction flask. Pd₂(dba)₃ (1.289 g) and potassium phosphate (5.98 g) were then added, and finally toluene (50 mL) was added. The mixture was reacted at 80° C. under N₂ atmosphere. After the reaction was completed, the reaction solution was washed twice with 50 mL of water. The organic layer was dried over anhydrous sodium sulfate and filtered under vacuum, and the filtrate was subjected to rotary evaporation to dryness, purified by silica gel column (PE-EA=95:5, v/v, 1500 mL), and further purified by C₁₈ reversed-phase column (10 nM aqueous ammonium acetate solution-acetonitrile=50%:50%, v/v) to give 480 mg of intermediate 2b.

MS (ESI, [M+H]⁺) m/z: 305.1.

¹H NMR (500 MHz, DMSO-d₆) δ 7.17 (t, J=7.8 Hz, 1H), 6.96 (dd, J=7.9, 0.8 Hz, 1H), 6.58 (dd, J=7.8, 0.9 Hz, 1H), 4.73 (t, J=5.2 Hz, 1H), 4.17-4.09 (m, 4H), 3.67 (dd, J=9.7, 7.5 Hz, 1H), 3.60 (ddd, J=9.5, 8.1, 5.0 Hz, 1H), 3.56-3.35 (m, 4H), 2.45 (dq, J=14.0, 7.0 Hz, 1H), 2.10-2.01 (m, 1H), 1.76 (dq, J=12.2, 7.5 Hz, 1H), 1.18 (t, J=7.1 Hz, 3H).

### Step 2: Preparation of Intermediate 2c

Intermediate 2b (450 mg) and THF (40 mL) were first added to a reaction flask, acrylamide (126 mg) and potassium tert-butoxide (249 mg) were added at 0° C., and the mixture was slowly warmed to 10° C. under N₂ atmosphere and stirred. After the reaction was completed, 100 mL of a saturated ammonium chloride solution was added to the reaction solution at a low temperature, and the mixture was extracted twice with EA (50 mL). The organic layer was dried over anhydrous sodium sulfate and filtered under vacuum, the filtrate was subjected to rotary evaporation to dryness and purified by silica gel column (DCM:MeOH=95:5, v/v) to give 450 mg of intermediate 2c.

MS (ESI, [M+H]⁺) m/z: 330.1.

### Step 3: Preparation of Intermediate 2d

Intermediate 2c (440 mg) and DCM (30 mL) were added to a reaction flask in sequence, Dess-Martin reagent (1.7 g) was added with stirring, and the mixture was reacted at room temperature under N₂ atmosphere. After the reaction was completed, an aqueous Na₂S2O₃ solution (30 mL) was added at a low temperature to quench the reaction, and the organic layer separated by extraction was washed with an aqueous NaHCO₃ solution (30 mL). The separated organic layer was dried over anhydrous sodium sulfate and filtered under vacuum, and the resulting filtrate was subjected to rotary evaporation to dryness to give 342 mg of intermediate 2d, which was directly used in the next step.

### Step 4: Preparation of Compound 2

Intermediate 1l (250 mg), intermediate 2d (171 mg), DCM (10 mL), and glacial acetic acid (31.4 mg, 0.030 mL) were added to a reaction flask in sequence. After the mixture was stirred at room temperature for 20 min, sodium cyanoborohydride (197 mg) was added, and the mixture was stirred at room temperature. After the reaction was completed, 20 mL of a saturated NaHCO₃ solution was added to the reaction solution, and the mixture was extracted twice with 20 mL of DCM-MeOH (10:1). The extracts were combined, dried over anhydrous sodium sulfate, and filtered under vacuum, and the resulting filtrate was subjected to rotary evaporation to dryness and purified by C₁₈ reversed-phase column (10 nM aqueous ammonium acetate solution-acetonitrile=50%:50%, v/v) to give 60 mg of compound 2.

Q-TOF (ESI, [M+H]⁺) m/z: 790.4157.

¹H NMR (500 MHz, DMSO-d₆) δ 11.17 (s, 1H), 11.07 (s, 1H), 7.74 (d, J=2.8 Hz, 1H), 7.65 (s, 1H), 7.52-7.47 (m, 2H), 7.32 (d, J=2.8 Hz, 1H), 7.20-7.12 (m, 3H), 6.97 (d, J=7.9 Hz, 1H), 6.59 (d, J=7.7 Hz, 1H), 4.53 (dd, J=11.6, 5.0 Hz, 1H), 4.37 (d, J=12.6 Hz, 1H), 4.28 (d, J=13.3 Hz, 1H), 3.74 (ddd, J=9.9, 7.2, 2.6 Hz, 1H), 3.67-3.59 (m, 2H), 3.55 (qd, J=7.4, 3.7 Hz, 1H), 3.40-3.32 (m, 3H), 3.30-3.23 (m, 3H), 3.07 (d, J=10.9 Hz, 1H), 3.04-2.90 (m, 3H), 2.77 (td, J=11.8, 6.0 Hz, 1H), 2.67-2.55 (m, 2H), 2.48-2.34 (m, 4H), 2.21-1.97 (m, 4H), 1.85-1.52 (m, 9H).

¹³C NMR (126 MHz, DMSO) δ 173.58, 171.99, 169.70, 160.77, 157.13, 154.01, 153.30, 150.98, 140.44, 137.77, 134.30, 127.46, 125.70, 122.03, 120.33, 118.64, 114.69, 111.16, 108.46, 62.25, 55.11, 54.25, 54.10, 49.11, 48.88, 47.20, 45.46, 44.86, 41.79, 36.13, 33.90, 31.55, 31.41, 29.70, 28.25, 24.50, 23.23.

### Example 3: Synthesis of Compound 3

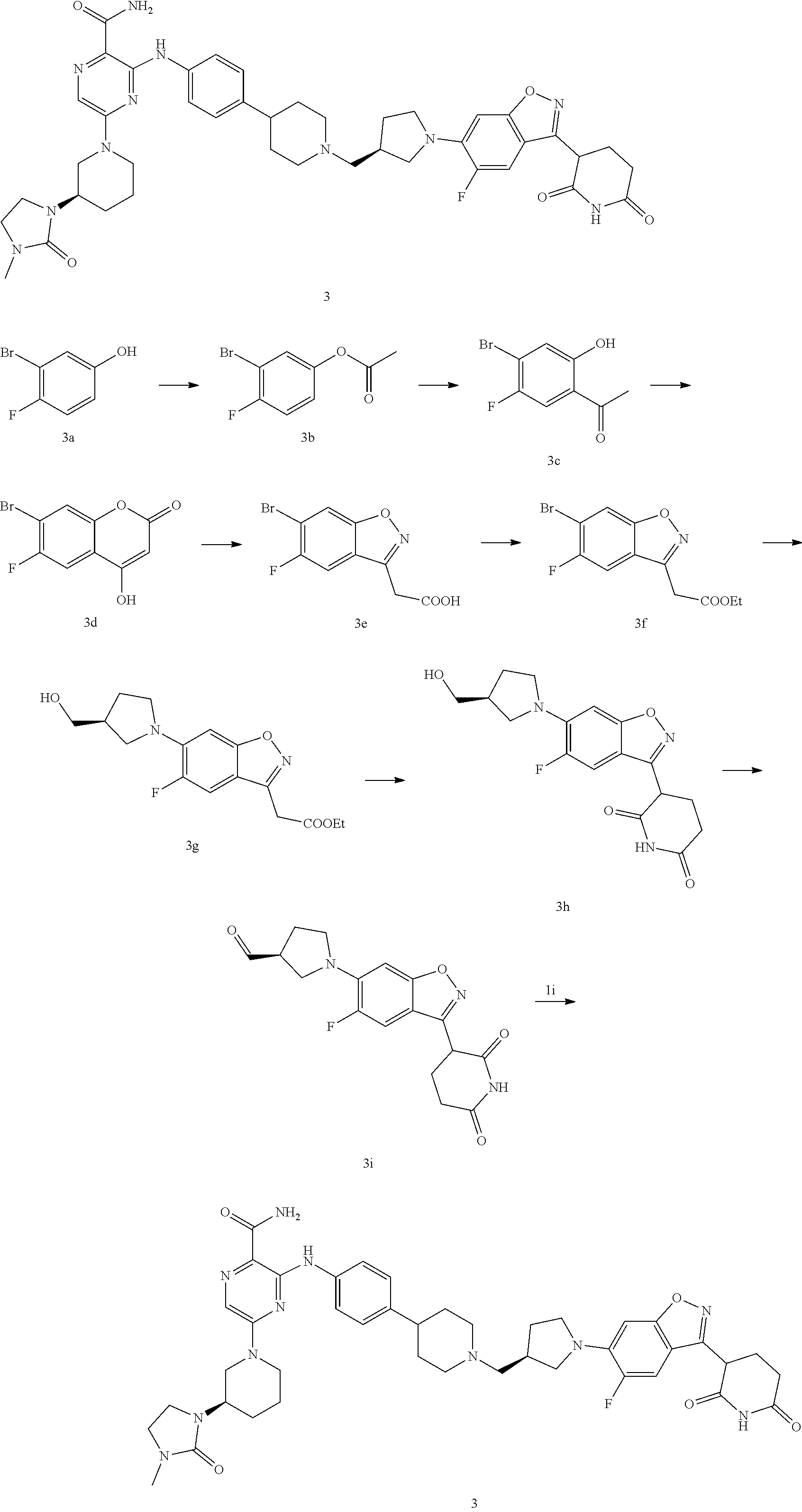

### Step 1: Preparation of Intermediate 3b

In a single-necked flask, intermediate 3a (20 g) was dissolved in dichloromethane (200 mL). Triethylamine (15.89 g, 21.89 mL) was added at 0° C., and acetyl chloride (12.33 g) was slowly added dropwise. After the addition was completed, the mixture was reacted at room temperature for 1 h. After the reaction was completed, 300 mL of dichloromethane was added to the system for dilution, and a 3 M aqueous hydrochloric acid solution was then added to adjust the pH of the system to weak acidity. Extraction and liquid separation were performed, and the organic phase was collected. The pH of the organic phase was adjusted to weak alkalinity with a saturated aqueous sodium bicarbonate solution. Extraction and liquid separation were performed, and the organic phase was collected. The organic phase was dried over anhydrous sodium sulfate, filtered and concentrated by evaporation at reduced pressure to remove the solvent, thus giving intermediate 3b (28.5 g).

¹H NMR (500 MHz, DMSO-d₆) δ 7.60 (dd, J=5.9, 2.7 Hz, 1H), 7.44 (t, J=8.7 Hz, 1H), 7.23 (ddd, J=8.9, 4.1, 2.8 Hz, 1H), 2.26 (s, 3H).

### Step 2: Preparation of Intermediate 3c

Intermediate 3b (8.61 g) and aluminum trichloride (8.92 g) were added to a single-necked flask in sequence, and the mixture was heated to 170° C. and reacted for 2 h. The reaction solution was cooled to room temperature, 50 mL of dichloromethane was added, and about 500 mL of 3 M hydrochloric acid was slowly added. Liquid separation was performed, the organic phase was collected, and the aqueous phase was then extracted 2 times with 250 mL of dichloromethane. The organic phases were combined, dried over anhydrous sodium sulfate, filtered and concentrated by evaporation at reduced pressure to remove the solvent, thus giving intermediate 3c (11.8 g).

MS(ESI, [M−H]⁺) m/z: 231.0

¹H NMR (500 MHz, DMSO-d₆) δ 11.64 (s, 1H), 7.79 (d, J=9.3 Hz, 1H), 7.33 (d, J=5.8 Hz, 1H), 2.62 (s, 3H).

### Step 3: Preparation of Intermediate 3d

Intermediate 3c (10 g), diethyl carbonate (25.3 g), and toluene (100 mL) were added to a single-necked flask in sequence. The reaction solution was cooled to 0° C. and sodium hydride (8.58 g) was added in portions. The mixture was first heated to 90° C. and then heated to 120° C. and reacted for 5 h. After the reaction was completed, the reaction solution was cooled to room temperature and slowly poured into 1 L of stirred ice water. The mixture was extracted with 500 mL of ethyl acetate, and the organic phase was discarded. The aqueous phase was adjusted to pH=3 with 3 N hydrochloric acid, the mixture was extracted 3 times with 300 mL of ethyl acetate, and the organic phases were combined. The organic phase was dried over anhydrous sodium sulfate, filtered and concentrated by evaporation at reduced pressure to remove the solvent, thus giving intermediate 3d (9.8 g).

MS(ESI, [M−H]⁺) m/z: 256.95.

¹H NMR (500 MHz, DMSO-d₆) δ 12.86 (s, 1H), 7.88 (d, J=5.6 Hz, 1H), 7.67 (d, J=8.5 Hz, 1H), 5.65 (s, 1H).

### Step 4: Preparation of Intermediate 3e

Intermediate 3d (9 g), hydroxylamine hydrochloride (7.95 g), sodium ethoxide (2.224 g), and ethanol (50 mL) were added to a single-necked flask in sequence, and the mixture was heated to 85° C. and reacted overnight. The reaction solution was cooled to room temperature, 3 N hydrochloric acid was added to adjust the pH to 5, the mixture was concentrated by evaporation at reduced pressure to remove the solvent, 100 L of water was added, the mixture was cooled, and meanwhile the pH was adjusted to 3 with 3 N hydrochloric acid. The mixture was stirred for 30 min and filtered. The filter cake was collected and dried to give intermediate 3e (8.1 g).

MS(ESI, [M+H]⁺) m/z: 273.86.

¹H NMR (500 MHz, DMSO-d₆) δ 12.97 (s, 1H), 8.30 (d, J=5.3 Hz, 1H), 7.93 (d, J=7.9 Hz, 1H), 4.12 (s, 2H).

### Step 5: Preparation of Intermediate 3f

Intermediate 3e (6 g), ethanol (40 mL), and sulfuric acid (9.27 g, 5.04 mL) were added to a single-necked flask in sequence, and the mixture was heated to 90° C. and reacted for 2 h. The reaction solution was cooled to room temperature and concentrated by evaporation at reduced pressure to remove the solvent, and 100 mL of ethyl acetate and 100 mL of water were added to the residue for dilution. A saturated aqueous sodium bicarbonate solution was added to adjust the pH to 7, and the organic phase was separated. The aqueous phase was extracted 2 times with 100 mL of ethyl acetate, and the organic phases were combined, dried over anhydrous sodium sulfate, filtered and concentrated by evaporation at reduced pressure to remove the solvent, thus giving intermediate 3f (5.4 g).

MS(ESI, [M+H]⁺) m/z: 302.21.

¹H NMR (500 MHz, DMSO-d₆) δ 8.32 (d, J=5.3 Hz, 1H), 7.94 (d, J=8.0 Hz, 1H), 4.22 (s, 2H), 4.16 (q, J=7.1 Hz, 2H), 1.21 (t, J=7.1 Hz, 3H).

### Step 6: Preparation of Intermediate 3g

Intermediate 3f (4 g), (S)-pyrrolidine-3-methanol (2.68 g), palladium acetate (0.595 g), potassium phosphate (8.43 g), and 1,4-dioxane (20 mL) were added to a single-necked flask in sequence, and the mixture was heated to 100° C. and reacted under N₂ atmosphere overnight. The reaction solution was cooled to room temperature and concentrated by evaporation at reduced pressure to remove the solvent, and the crude product was separated by silica gel column chromatography (eluent: petroleum ether/ethyl acetate=1:1, v/v) to give the target intermediate 3g (0.5 g).

MS(ESI, [M+H]⁺) m/z: 323.11.

¹H NMR (500 MHz, DMSO-d₆) δ 7.45 (d, J=13.3 Hz, 1H), 6.81 (d, J=7.0 Hz, 1H), 4.73 (t, J=5.2 Hz, 1H), 4.13 (q, J=7.2 Hz, 2H), 4.03 (s, 2H), 3.52 (ddd, J=10.5, 7.4, 3.2 Hz, 1H), 3.49-3.38 (m, 4H), 3.27 (ddd, J=10.0, 6.7, 3.1 Hz, 1H), 2.41 (td, J=14.9, 14.1, 7.9 Hz, 1H), 2.01 (dtd, J=12.2, 7.9, 7.5, 5.3 Hz, 1H), 1.72 (dq, J=12.2, 7.7 Hz, 1H), 1.19 (t, J=7.1 Hz, 3H).

### Step 7: Preparation of Intermediate 3h

Intermediate 3g (500 mg), acrylamide (98 mg), and anhydrous tetrahydrofuran (5 mL) were added to a three-necked flask in sequence. Potassium tert-butoxide (233 mg) was slowly added at −15° C., and the mixture was reacted at −15° C. for 2 h. A saturated aqueous ammonium chloride solution was added dropwise to the reaction solution to quench the reaction, and the mixture was extracted with 50 mL of ethyl acetate. The organic phase was separated. The aqueous phase was extracted 3 times with 50 mL of ethyl acetate, the organic phases were combined, dried over anhydrous sodium sulfate, filtered and concentrated by evaporation at reduced pressure to remove the solvent, thus giving intermediate 3h (120 mg).

MS(ESI, [M−H]⁺) m/z: 345.9.

¹H NMR (500 MHz, DMSO-d₆) δ 11.03 (s, 1H), 7.52 (d, J=13.3 Hz, 1H), 6.82 (d, J=6.9 Hz, 1H), 4.72 (t, J=5.3 Hz, 1H), 4.41 (dd, J=11.6, 5.0 Hz, 1H), 3.45 (td, J=11.7, 6.2 Hz, 5H), 2.72 (ddd, J=17.8, 12.3, 5.7 Hz, 1H), 2.57 (dt, J=17.3, 4.1 Hz, 1H), 2.39 (tq, J=13.0, 7.7, 6.9 Hz, 2H), 2.14 (dt, J=13.5, 4.6 Hz, 1H), 2.00 (tq, J=13.4, 8.3, 7.1 Hz, 2H), 1.77-1.67 (m, 1H).

### Step 8: Preparation of Intermediate 3i

Intermediate 3h (80 mg), acetonitrile (5 mL), and IBX (129 mg) were added to a single-necked flask in sequence, and the mixture was heated to 85° C. and reacted for 1 h. The reaction solution was cooled to room temperature and filtered, and the filtrate was collected. The resulting solution (containing intermediate 3i) was directly used in the next step.

### Step 9: Preparation of Compound 3

Intermediate 3i (the solution of intermediate 3i obtained in the previous step), intermediate 1l (60 mg), and methanol (1 mL) were added to a single-necked flask in sequence, and 1 drop of acetic acid was added dropwise. After the mixture was stirred at room temperature for 1 h, sodium cyanoborohydride (23.63 mg) was added, and the mixture was reacted at room temperature for 2 h. The mixture was concentrated by evaporation at reduced pressure to remove the solvent and purified by preparative liquid chromatography. Compound 3 (25 mg) was obtained.

MS(ESI, [M+H]⁺) m/z: 808.6.

¹H NMR (500 MHz, DMSO-d₆) δ 11.18 (s, 1H), 11.03 (s, 1H), 7.75 (s, 1H), 7.66 (s, 1H), 7.51 (dd, J=18.1, 10.6 Hz, 3H), 7.33 (s, 1H), 7.17 (d, J=8.1 Hz, 2H), 6.84 (d, J=7.0 Hz, 1H), 4.51-4.33 (m, 2H), 4.28 (d, J=13.3 Hz, 1H), 3.60 (dd, J=25.9, 10.7 Hz, 2H), 3.47 (d, J=9.5 Hz, 2H), 3.26 (t, J=8.1 Hz, 3H), 2.98 (dt, J=32.9, 12.0 Hz, 4H), 2.73 (s, 4H), 2.57 (dt, J=17.3, 4.1 Hz, 2H), 2.42-2.30 (m, 2H), 2.21-1.96 (m, 4H), 1.91-1.45 (m, 10H).

¹³C NMR (126 MHz, DMSO-d₆) δ 173.59, 172.00, 169.71, 161.59, 160.79, 156.67, 154.02, 152.29, 150.98, 148.61, 127.45, 120.32, 118.68, 114.69, 109.76, 109.50, 106.13, 93.49, 54.85, 54.21, 49.77, 49.10, 47.19, 45.46, 44.85, 39.00, 35.05, 34.96, 33.69, 31.76, 31.62, 31.55, 31.45, 30.86, 30.30, 29.71, 29.46, 28.24, 24.50, 23.01.

### Example 4: Synthesis of Compound 4

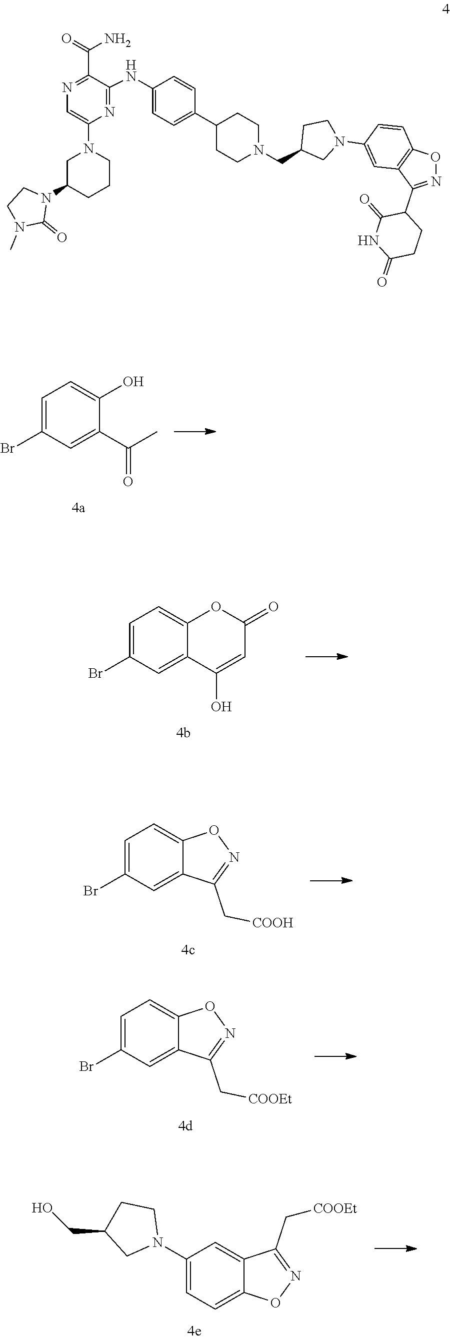 [SMILES: CN1CCN([C@@H]2CCCN(c3cnc(C(N)=O)c(Nc4ccc(C5CCN(C[C@H]6CCN(c7ccc8onc(C9CCC(=O)NC9=O)c8c7)C6)CC5)cc4)n3)C2)C1=O ; CC(=O)c1cc(Br)ccc1O ; O=c1cc(O)c2cc(Br)ccc2o1 ; O=C(O)Cc1noc2ccc(Br)cc12 ; CCOC(=O)Cc1noc2ccc(Br)cc12 ; CCOC(=O)Cc1noc2ccc(N3CC[C@H](CO)C3)cc12]

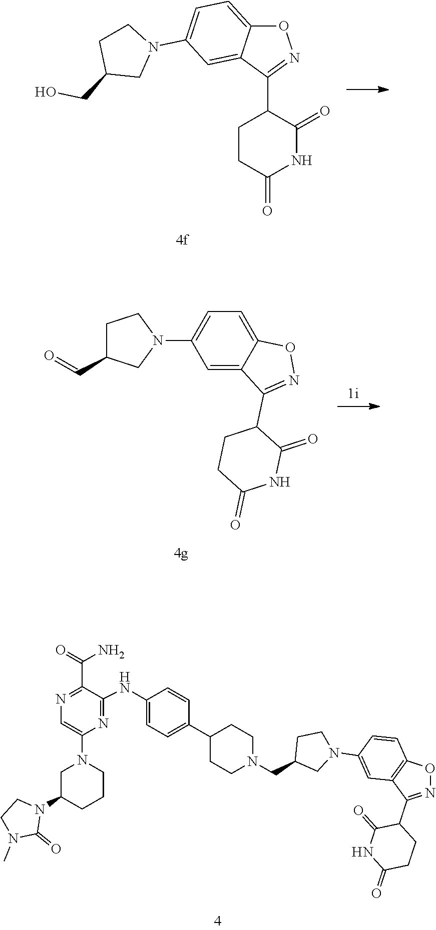 [SMILES: O=C1CCC(c2noc3ccc(N4CC[C@H](CO)C4)cc23)C(=O)N1 ; O=C[C@H]1CCN(c2ccc3onc(C4CCC(=O)NC4=O)c3c2)C1 ; CN1CCN([C@@H]2CCCN(c3cnc(C(N)=O)c(Nc4ccc(C5CCN(C[C@H]6CCN(c7ccc8onc(C9CCC(=O)NC9=O)c8c7)C6)CC5)cc4)n3)C2)C1=O]

### Step 1: Preparation of Intermediate 4b

4a (25 g), diethyl carbonate (68.7 g), and toluene (200 mL) were added to a reaction flask in sequence. The reaction solution was cooled to 0° C. and sodium hydride (23.25 g) was added in portions. The mixture was heated to 120° C. and reacted for 5 h. The reaction was stopped, the reaction solution was cooled to room temperature and slowly poured into 1.5 L of stirred ice water. The mixture was extracted with 400 mL of ethyl acetate. The aqueous phase was adjusted to pH=3 with 3 N hydrochloric acid, the mixture was extracted 3 times with 400 mL of ethyl acetate, and the organic phases were combined. The organic phase was dried over anhydrous sodium sulfate, filtered and concentrated by evaporation at reduced pressure to remove the solvent, thus giving 25 g of intermediate 4b.

MS(ESI, [M−H]⁺) m/z: 239.0

¹H NMR (500 MHz, DMSO-d₆) δ 12.78 (s, 1H), 7.90 (d, J=2.4 Hz, 1H), 7.80 (dd, J=8.8, 2.5 Hz, 1H), 7.36 (d, J=8.8 Hz, 1H), 5.62 (s, 1H).

### Step 2: Preparation of Intermediate 4c

4b (13 g), hydroxylamine hydrochloride (7.50 g), sodium methoxide (5.83 g), and ethanol (100 mL) were added to a reaction flask in sequence, and the reaction solution was heated to 85° C. and reacted for 15.5 h under N₂ atmosphere. The reaction solution was cooled to room temperature and concentrated by evaporation at reduced pressure to remove the solvent. After 200 mL of water was added to the residue, 3 M hydrochloric acid was added. The mixture was filtered, and the filter cake was purified by silica gel column chromatography (eluent: DCM:CH₃OH=9:1, v/v) to give 5.5 g of intermediate 4c.

MS(ESI, [M−H]⁺) m/z: 254.1.

¹H NMR (500 MHz, DMSO-d₆) δ 12.96 (s, 1H), 8.14 (d, J=2.0 Hz, 1H), 7.81 (dd, J=8.9, 2.0 Hz, 1H), 7.75 (d, J=8.8 Hz, 1H), 4.13 (s, 2H).

### Step 3: Preparation of Intermediate 4d

4c (5.5 g), ethanol (50 mL), and concentrated sulfuric acid (12.64 g, 6.87 mL) were added to a reaction flask in sequence, and the mixture was heated to 90° C. and reacted for 15 h. The reaction solution was cooled to room temperature and concentrated by evaporation at reduced pressure to remove the solvent, and 250 mL of ethyl acetate and 250 mL of water were added to the residue. A saturated aqueous sodium bicarbonate solution was added to adjust the pH to 7, and the organic phase was separated. The aqueous phase was extracted 2 times with 280 mL of ethyl acetate, the organic phases were combined, dried over anhydrous sodium sulfate, filtered and concentrated by evaporation at reduced pressure to remove the solvent, thus giving 5.65 g of intermediate 4d.

MS(ESI, [M+H]⁺) m/z: 282.0

¹H NMR (500 MHz, DMSO-d₆) δ 8.16 (dd, J=2.0, 0.6 Hz, 1H), 7.82 (dd, J=8.9, 1.9 Hz, 1H), 7.76 (dd, J=8.9, 0.6 Hz, 1H), 4.23 (s, 2H), 4.15 (q, J=7.1 Hz, 2H), 1.21 (t, J=7.1 Hz, 3H).

### Step 4: Preparation of Intermediate 4e

(S)-Pyrrolidine-3-methanol (3.52 g) and intermediate 4d (3.3 g) were added in sequence to a reaction flask. Pd₂(dba)₃ (2.127 g) and potassium phosphate (9.86 g) were then added, and finally toluene (100 mL) was added. The mixture was heated to 80° C. for 3.5 h under N₂ atmosphere. The reaction solution was cooled to room temperature with stirring and filtered, and the filtrate was purified by silica gel column chromatography (PE:EA=3:2, v/v) to give 0.8 g of intermediate 4e.

MS(ESI, [M+H]⁺) m/z: 305.2.

¹H NMR (500 MHz, DMSO-d₆) δ 7.54 (d, J=9.0 Hz, 1H), 6.98 (dd, J=9.0, 2.4 Hz, 1H), 6.70 (d, J=2.3 Hz, 1H), 4.72 (t, J=5.2 Hz, 1H), 4.14 (q, J=7.1 Hz, 2H), 3.51-3.37 (m, 2H), 3.31-3.21 (m, 2H), 3.06 (dd, J=9.3, 6.2 Hz, 1H), 2.48-2.40 (m, 1H), 2.06 (dtd, J=12.3, 7.3, 4.9 Hz, 1H), 1.76 (dq, J=12.3, 7.5 Hz, 1H), 1.20 (t, J=7.1 Hz, 3H).

### Step 5: Preparation of Intermediate 4f

Intermediate 4e (800 mg) and THF (20 mL) were added to a reaction flask. Acrylamide (185 mg) and potassium tert-butoxide (292 mg) were then added in sequence at 0° C., and the mixture was reacted for 3 h under an ice-water bath under N₂ atmosphere. The reaction solution was added dropwise to an aqueous ammonium chloride solution, and the reaction was neutralized. After 100 mL of ethyl acetate was added, the mixture was extracted, the organic phase was washed with saturated brine, dried over anhydrous sodium sulfate and filtered, and the filtrate was purified by silica gel column chromatography (eluent EA) to give 0.4 g of intermediate 4f.

MS(ESI, [M+H]⁺) m/z: 330.2.

¹H NMR (500 MHz, DMSO-d₆) δ 11.10-10.97 (m, 1H), 7.55 (d, J=9.0 Hz, 1H), 6.98 (dd, J=9.1, 2.3 Hz, 1H), 6.68 (t, J=2.5 Hz, 1H), 4.72 (td, J=5.2, 1.6 Hz, 1H), 4.52 (ddd, J=11.4, 5.0, 1.2 Hz, 1H), 3.51-3.39 (m, 2H), 3.31-3.18 (m, 2H), 3.05 (ddd, J=17.5, 9.5, 6.2 Hz, 1H), 2.75 (ddd, J=16.9, 11.5, 5.4 Hz, 1H), 2.62-2.51 (m, 2H), 2.45 (dt, J=13.9, 7.0 Hz, 1H), 2.19-2.01 (m, 2H), 1.76 (ddt, J=12.0, 7.6, 3.4 Hz, 1H), 1.19 (dt, J=11.6, 7.1 Hz, 1H).

### Step 6: Synthesis of Compound 4

Intermediate 4f (200 mg) and DCM (2 mL) were added to a reaction flask, Dess-Martin periodinane (515 mg) was added at 0° C. reaction, and the mixture was reacted at room temperature for 1 h. The reaction solution was filtered. After the filtrate was evaporated under reduced pressure to remove part of the solvent, methanol (5 mL), sodium cyanoborohydride (76 mg), and intermediate 1l (291 mg) were directly added, and the mixture was reacted at room temperature for 2 h. The reaction solution was purified by silica gel column chromatography (DCM:CH₃OH=10:1, v/v) and then purified by C18 reversed-phase column (H₂O (1 vol % ammonium acetate):CH₃CN=40%:60%, v/v) to give 0.44 g of compound 4.

MS(ESI, [M+H]⁺) m/z: 790.46.

¹H NMR (500 MHz, DMSO-d₆) δ 11.20 (s, 1H), 11.05 (s, 1H), 7.76 (d, J=2.9 Hz, 1H), 7.66 (s, 1H), 7.56 (d, J=9.0 Hz, 1H), 7.50 (d, J=8.1 Hz, 2H), 7.33 (d, J=2.8 Hz, 1H), 7.17 (d, J=8.1 Hz, 2H), 6.98 (dd, J=9.2, 2.3 Hz, 1H), 6.70 (d, J=2.4 Hz, 1H), 4.54 (dd, J=11.5, 5.0 Hz, 1H), 4.32 (dd, J=36.1, 11.7 Hz, 2H), 3.62 (tt, J=11.3, 4.0 Hz, 1H), 3.44-3.35 (m, 2H), 3.26 (ddd, J=16.0, 13.1, 7.5 Hz, 4H), 3.09-2.90 (m, 5H), 2.80-2.74 (m, 1H), 2.72 (s, 3H), 2.67-2.51 (m, 3H), 2.47-2.28 (m, 3H), 2.23-2.09 (m, 2H), 2.07-1.92 (m, 2H), 1.87-1.46 (m, 10H).

¹³C NMR (126 MHz, DMSO-d₆) δ 173.67, 172.08, 169.70, 160.78, 156.52, 156.26, 154.02, 150.97, 145.56, 137.82, 127.44, 122.01, 121.98, 120.28, 118.66, 117.61, 114.70, 110.26, 100.53, 62.34, 55.38, 54.42, 53.24, 49.11, 48.14, 47.21, 45.45, 44.85, 39.01, 36.21, 33.75, 31.57, 31.47, 29.98, 28.22, 24.47, 22.96.

### Example 5: Synthesis of Compound 5

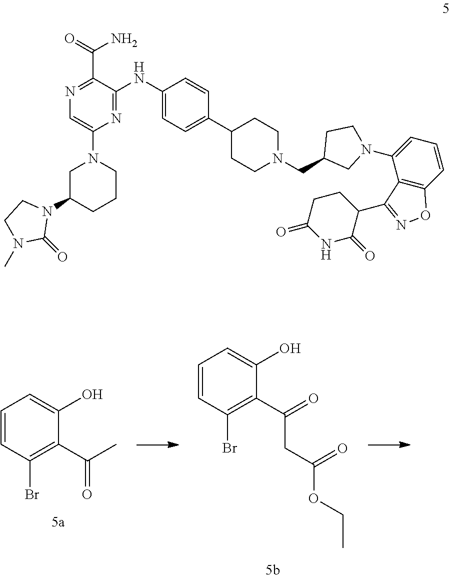 [SMILES: CN1CCN([C@@H]2CCCN(c3cnc(C(N)=O)c(Nc4ccc(C5CCN(C[C@H]6CCN(c7cccc8onc(C9CCC(=O)NC9=O)c78)C6)CC5)cc4)n3)C2)C1=O ; CC(=O)c1c(O)cccc1Br ; CCOC(=O)CC(=O)c1c(O)cccc1Br]

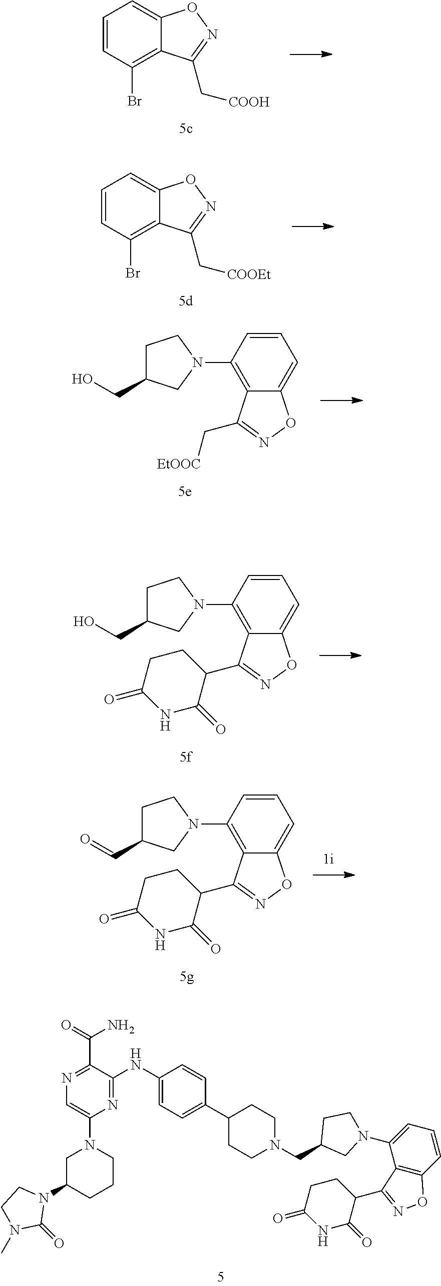 [SMILES: O=C(O)Cc1noc2cccc(Br)c12 ; CCOC(=O)Cc1noc2cccc(Br)c12 ; CCOC(=O)Cc1noc2cccc(N3CC[C@H](CO)C3)c12 ; O=C1CCC(c2noc3cccc(N4CC[C@H](CO)C4)c23)C(=O)N1 ; O=C[C@H]1CCN(c2cccc3onc(C4CCC(=O)NC4=O)c23)C1 ; CN1CCN([C@@H]2CCCN(c3cnc(C(N)=O)c(Nc4ccc(C5CCN(C[C@H]6CCN(c7cccc8onc(C9CCC(=O)NC9=O)c78)C6)CC5)cc4)n3)C2)C1=O]

### Step 1: Preparation of Intermediate 5b

1-(2-Bromo-6-hydroxyphenyl)ethan-1-one (9.5 g), diethyl carbonate (26.1 g), and toluene (200 mL) were added to a reaction flask in sequence. The reaction solution was cooled to 0° C., and sodium hydride (8.83 g, 60%, 221 mmol) was added in portions. The mixture was heated to 120° C. and reacted overnight, cooled to room temperature, and slowly poured into 1.5 L of stirred ice water. The mixture was extracted with 400 mL of ethyl acetate. The aqueous phase was adjusted to pH=3 with 3 N hydrochloric acid and extracted 3 times with 400 mL of ethyl acetate, the organic phases were combined, washed once with saturated brine, dried over anhydrous sodium sulfate, filtered, and concentrated by evaporation at reduced pressure to remove the solvent, thus giving intermediate 5b (12.4 g).

MS(ESI, [M−H]⁻) m/z: 285.0.

¹H NMR (500 MHz, DMSO-d6) δ 10.60 (s, 1H), 7.19 (t, J=8.2 Hz, 1H), 7.09 (dd, J=7.9, 0.9 Hz, 1H), 6.91 (dd, J=8.2, 0.9 Hz, 1H), 4.09 (q, J=7.1 Hz, 2H), 3.87 (s, 2H), 1.20-1.14 (m, 3H).

### Step 2: Preparation of Intermediate 5c

Intermediate 5b (11.2 g), MeOH (200 mL), hydroxylamine hydrochloride (9.49 g), and sodium acetate (11.20 g) were added to a reaction flask in sequence, and the mixture was heated to 80° C. and reacted for 3 h. The reaction solution was cooled to room temperature, 3 N hydrochloric acid was added to adjust the pH to 5, the mixture was concentrated by evaporation at reduced pressure to remove the solvent, 1 L of water was added, the reaction flask was placed under an ice-water bath to cool, and meanwhile the pH was adjusted to 3 with 3 N hydrochloric acid. The mixture was stirred for 30 min and filtered. The filter cake was collected and dried to give intermediate 5c (8.0 g).

¹H NMR (500 MHz, DMSO-d6) δ 12.99 (s, 1H), 7.81 (dd, J=8.2, 0.8 Hz, 1H), 7.66-7.55 (m, 2H), 4.15 (s, 2H).

### Step 3: Preparation of Intermediate 5d

Intermediate 5c (8 g), ethanol (100 mL), and concentrated sulfuric acid (6.13 g, 3.33 mL) were added to a reaction flask in sequence, and the mixture was heated to 90° C. and reacted overnight. The reaction solution was cooled to room temperature and concentrated by evaporation at reduced pressure to remove the solvent, and 250 mL of ethyl acetate and 250 mL of water were added to the residue for dilution. A saturated aqueous sodium bicarbonate solution was added to adjust the pH to 7, and the organic phase was separated. The aqueous phase was extracted 2 times with 250 mL of ethyl acetate to give intermediate 5d (8.64 g).

¹H NMR (500 MHz, DMSO-d6) δ 7.83 (dd, J=8.1, 0.9 Hz, 1H), 7.69-7.56 (m, 2H), 4.25 (s, 2H), 4.16 (q, J=7.1 Hz, 2H), 1.20 (t, J=7.1 Hz, 3H).

### Step 4: Preparation of Intermediate 5e

Intermediate 5d (500 mg), (S)-pyrrolidin-3-ylmethanol (267 mg), palladium acetate (39.5 mg), and potassium phosphate (747 mg) were added to a reaction flask in sequence, a solvent 1,4-dioxane (30 mL) was added, and the mixture was heated to 100° C. and reacted under nitrogen atmosphere overnight. The above reaction procedure was repeated 4 times. After the reaction was completed, the mixtures were cooled to room temperature, combined, filtered under vacuum, concentrated, and purified by silica gel column chromatography to give intermediate 5e (240 mg).

MS(ESI, [M+H]⁺) m/z: 305.0.

¹H NMR (500 MHz, DMSO-d6) δ 7.44 (t, J=8.0 Hz, 1H), 7.15 (d, J=8.2 Hz, 1H), 6.73 (d, J=7.8 Hz, 1H), 4.68 (t, J=5.2 Hz, 1H), 4.18 (s, 2H), 4.10 (q, J=7.1 Hz, 2H), 3.46-3.37 (m, 2H), 3.30-3.20 (m, 3H), 3.08-3.01 (m, 1H), 2.45-2.33 (m, 1H), 2.04-1.94 (m, 1H), 1.69-1.58 (m, 1H), 1.16 (t, J=7.1 Hz, 3H).

### Step 5: Preparation of Intermediate 5f

Intermediate 5e (120 mg), acrylamide (32.2 mg), and THF (5 mL) were added to a reaction flask in sequence. After the mixture was cooled to an internal temperature of −10° C., a potassium tert-butoxide solution (88 mg, 0.784 mL, 0.784 mmol) was slowly added, and the mixture was reacted at −10° C. After the reaction was completed, the reaction was quenched with saturated ammonium chloride. The mixture was extracted with 20 mL of EA, washed once with saturated brine, dried over anhydrous sodium sulfate, filtered, concentrated, and purified by silica gel column chromatography to give intermediate 5f (46 mg).

MS(ESI, [M+H]⁺) m/z: 330.0.

### Step 6: Preparation of Compound 5

Intermediate 5f (57 mg), acetonitrile (5 mL), and IBX (145 mg) were added to a reaction flask in sequence, and the mixture was reacted at 85° C. for 1 h. After the reaction was completed, the mixture was cooled to room temperature, and filtered under vacuum, and the filtrate was directly used in the next step (the filtrate contained 5 g of the intermediate). MeOH (5 mL) was added to the filtrate. Intermediate 1l (68.7 mg) and acetic acid (4.31 mg) were then added in sequence. After the mixture was stirred at room temperature for 20 min, sodium cyanoborohydride (27.1 mg) was added, and the mixture was reacted at room temperature for another 3 h. After the reaction was completed, 5 mL of a saturated sodium bicarbonate solution was added to the reaction solution. The mixture was extracted with DCM, washed once with saturated sodium chloride, dried over anhydrous sodium sulfate, filtered, concentrated, and purified by normal-phase silica gel column chromatography and C18 reversed-phase column chromatography in sequence to give compound 5 (28 mg).

MS(ESI, [M+H]⁺) m/z: 790.6.

¹H NMR (500 MHz, DMSO-d6) δ 11.19 (s, 1H), 11.08 (d, J=17.4 Hz, 1H), 7.83-7.73 (m, 1H), 7.66 (s, 1H), 7.58-7.47 (m, 3H), 7.39-7.27 (m, 2H), 7.19-7.13 (m, 2H), 7.04 (s, 1H), 4.63-4.56 (m, 1H), 4.42-4.25 (m, 2H), 3.65-3.56 (m, 1H), 3.31-3.14 (m, 6H), 3.12-2.88 (m, 5H), 2.81-2.73 (m, 1H), 2.69 (s, 3H), 2.63-2.54 (m, 1H), 2.48-2.22 (m, 5H), 2.16-1.90 (m, 3H), 1.89-1.43 (m, 9H).

### Example 6: Preparation of Compound 6

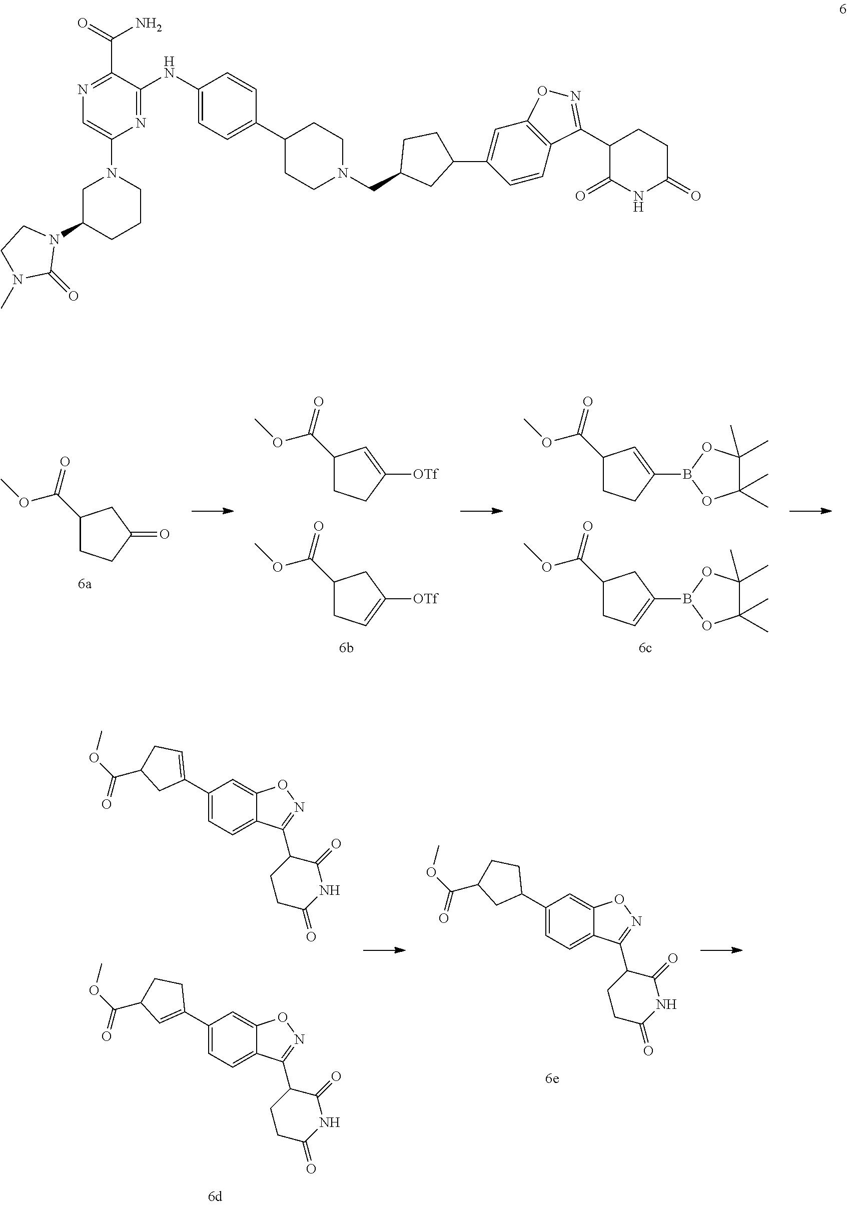

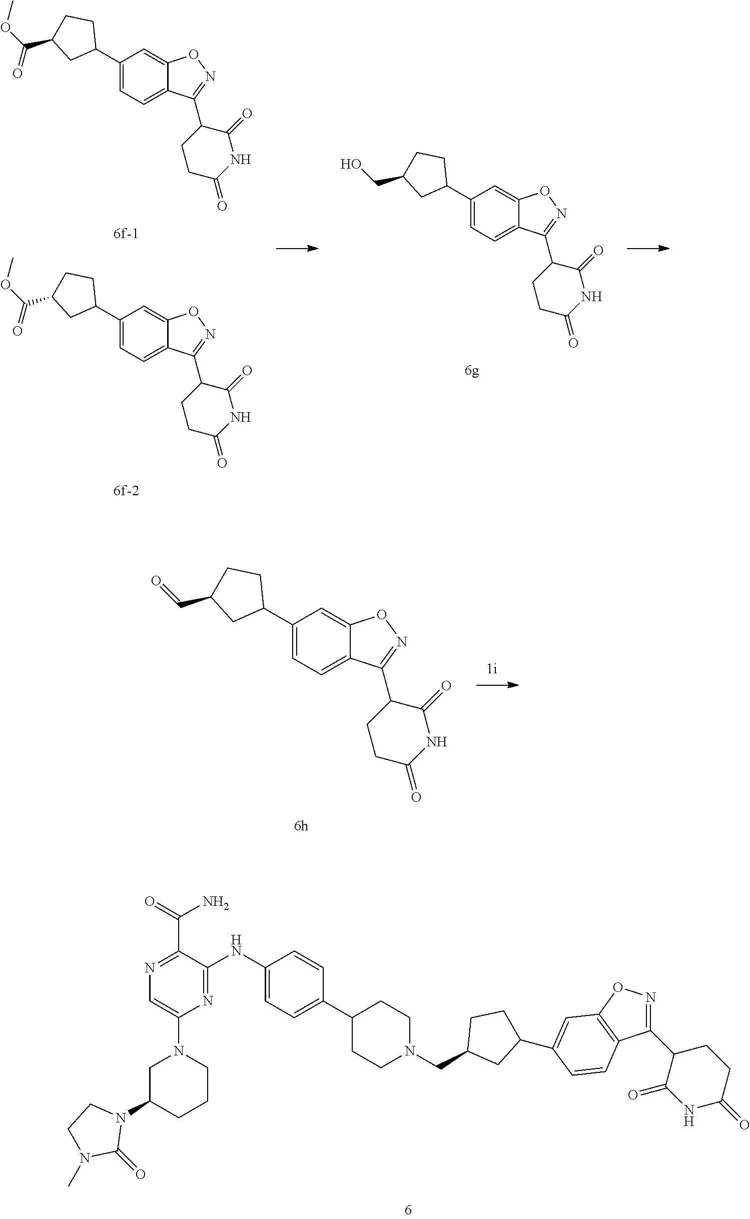 [SMILES: COC(=O)[C@@H]1CCC(c2ccc3c(C4CCC(=O)NC4=O)noc3c2)C1 ; COC(=O)[C@H]1CCC(c2ccc3c(C4CCC(=O)NC4=O)noc3c2)C1 ; O=C1CCC(c2noc3cc(C4CC[C@H](CO)C4)ccc23)C(=O)N1 ; O=C[C@H]1CCC(c2ccc3c(C4CCC(=O)NC4=O)noc3c2)C1 ; CN1CCN([C@@H]2CCCN(c3cnc(C(N)=O)c(Nc4ccc(C5CCN(C[C@H]6CCC(c7ccc8c(C9CCC(=O)NC9=O)noc8c7)C6)CC5)cc4)n3)C2)C1=O]

### Step 1: Preparation of Intermediate 6b

Intermediate 6a (3.0 g), DIPEA (5.73 g), and DCM (30 mL) were added to a reaction flask, trifluoromethanesulfonic anhydride (11.31 g) was slowly added under an ice bath, and then the mixture was slowly warmed to room temperature and reacted for 2 h. 100 mL of water and 100 mL of DCM were added to the reaction solution. The organic phase was separated, washed with 100 mL of a saturated sodium chloride solution, dried over anhydrous sodium sulfate, filtered, and concentrated. The concentrate was separated and purified by silica gel column chromatography to give mixture intermediate 6b (5.2 g).

¹H NMR (500 MHz, Chloroform-d) δ 5.59 (p, J=2.3 Hz, 1H), 3.73 (s, 3H), 3.29 (ddt, J=10.1, 8.6, 7.0 Hz, 1H), 2.98 (ddq, J=16.3, 6.7, 2.7 Hz, 1H), 2.76-2.70 (m, 2H), 2.37-2.26 (m, 1H).

### Step 2: Preparation of Intermediate 6c

Intermediate 6b (2.6 g), bis(pinacolato)diboron (2.89 g), potassium acetate (0.694 g), and 1,4-dioxane (30 mL) were added to a reaction flask in sequence, [1,1′-bis(diphenylphosphino)ferrocene]palladium dichloride dichloromethane complex (0.265 g) was added, and the mixture was purged with N₂ 3 times. The mixture was then heated to 85° C. and reacted for 2 h, and the heating was stopped. 200 mL of water was added to the reaction solution, and the mixture was extracted 2 times with 100 mL of EA. The combined organic layers were washed twice with 100 mL of a saturated sodium chloride solution. The mixture was washed, dried over anhydrous sodium sulfate, filtered, and concentrated. The concentrate was separated and purified by silica gel column chromatography to give mixture intermediate 6c (2.1 g).

### Step 3: Preparation of Intermediate 6d

Intermediate 6c (1.22 g), intermediate 1m (1.0 g), potassium carbonate (1.34 g), H₂O (3 mL), and 1,4-dioxane (15 mL) were added to a reaction flask in sequence, [1,1′-bis(diphenylphosphino)ferrocene]palladium dichloride dichloromethane complex (0.355 g) was added, and the mixture was purged with N₂ 3 times. The mixture was then heated to 70° C. and reacted for 2 h, and the heating was stopped. 200 mL of water was added to the reaction solution, and the mixture was extracted 2 times with 100 mL of DCM. The combined organic layers were washed twice with 100 mL of a saturated sodium chloride solution. The mixture was washed, dried over anhydrous sodium sulfate, filtered, and concentrated. The concentrate was separated and purified by silica gel column chromatography to give mixture intermediate 6d (0.328 g).

MS (ESI, [M−H]⁻) m/z: 353.2.

### Step 4: Preparation of Intermediate 6e

Intermediate 6d (0.32 g), palladium on carbon catalyst (0.032 g), and MeOH (10 mL) were added to a reaction flask in sequence, and the mixture was reacted at room temperature for 16 h under H₂ atmosphere. After the reaction was completed, the reaction solution was filtered, and the filtrate was concentrated to give intermediate 6e (0.17 g).

MS (ESI, [M−H]⁻) m/z: 355.2.

### Step 5: Preparation of Intermediates 6f-1 and 6f-2

Intermediate 6e (0.9 g) was resolved by preparative SFC to give intermediate 6f-1 (0.17 g) with S configuration and intermediate 6f-2 (0.1 g) with R configuration.

MS (ESI, [M−H]⁻) m/z: 355.2.

### Step 6: Preparation of Intermediate 6g

Intermediate 6f-1 (0.17 g) and THF (10 mL) were added to a reaction flask, lithium aluminum hydride (0.02 g) was slowly added under an ice bath, and then the mixture was slowly warmed to room temperature and reacted for 1 h. After the reaction was completed, a small amount of ice water was added to the reaction solution under an ice bath to quench the reaction, and 100 mL of DCM and 100 mL of water were then added. The mixture was filtered, and the organic phase of the filtrate was separated, washed with 100 mL of a saturated sodium chloride solution, dried over anhydrous sodium sulfate, filtered, and concentrated. The concentrate was separated and purified by silica gel column chromatography to give intermediate 6g (0.093 g).

MS (ESI, [M−H]⁻) m/z: 327.1.

### Step 7: Preparation of Intermediate 6h

Intermediate 6g (0.09 g) and dichloromethane (5 mL) were added to a reaction flask, Dess-Martin periodinane (0.233 g) was added, and the mixture was reacted at room temperature for 1 h. After the reaction was completed, dichloromethane (50 mL) and water (50 mL) were added to the system. The organic phase was separated, and the aqueous phase was extracted 2 times with 50 mL of dichloromethane. The organic phases were combined, dried over anhydrous sodium sulfate, filtered and concentrated to give intermediate 6h (0.08 g).

MS (ESI, [M−H]⁻) m/z: 325.1.

### Step 8: Preparation of Compound 6

Intermediate 6h (0.09 g), intermediate 1l (0.12 g), and methanol (5 mL) were added to a reaction flask, 1 drop of acetic acid was added, and sodium cyanoborohydride (0.032 g) was then added. The mixture was reacted at room temperature for 2 h. After the reaction was completed, 50 mL of dichloromethane and 50 mL of water were added to the system. The organic phase was separated, and the aqueous phase was extracted 2 times with 50 mL of dichloromethane. The organic phases were combined, dried over anhydrous sodium sulfate, filtered and concentrated. The concentrate was separated and purified by silica gel column chromatography to give compound 6 (0.08 g).

MS (ESI, [M+H]⁺) m/z: 789.5.

¹H NMR (500 MHz, DMSO-d₆) δ 11.18 (s, 1H), 11.09 (s, 1H), 7.75 (d, J=8.6 Hz, 2H), 7.64 (d, J=17.9 Hz, 2H), 7.49 (d, J=8.0 Hz, 2H), 7.32 (d, J=8.2 Hz, 2H), 7.16 (d, J=8.1 Hz, 2H), 4.56 (dd, J=11.9, 5.0 Hz, 1H), 4.39-4.33 (m, 1H), 4.31-4.24 (m, 1H), 3.64-3.58 (m, 1H), 3.27-3.22 (m, 4H), 3.05-2.91 (m, 4H), 2.78 (td, J=11.9, 6.1 Hz, 1H), 2.70 (s, 3H), 2.64-2.59 (m, 1H), 2.46 (s, 2H), 2.39-2.31 (m, 2H), 2.21 (ddt, J=13.7, 9.5, 5.7 Hz, 3H), 2.12-2.01 (m, 3H), 1.90 (s, 3H), 1.84-1.74 (m, 8H), 1.55 (d, J=12.6 Hz, 2H).

### Example 7: Preparation of Compound 7

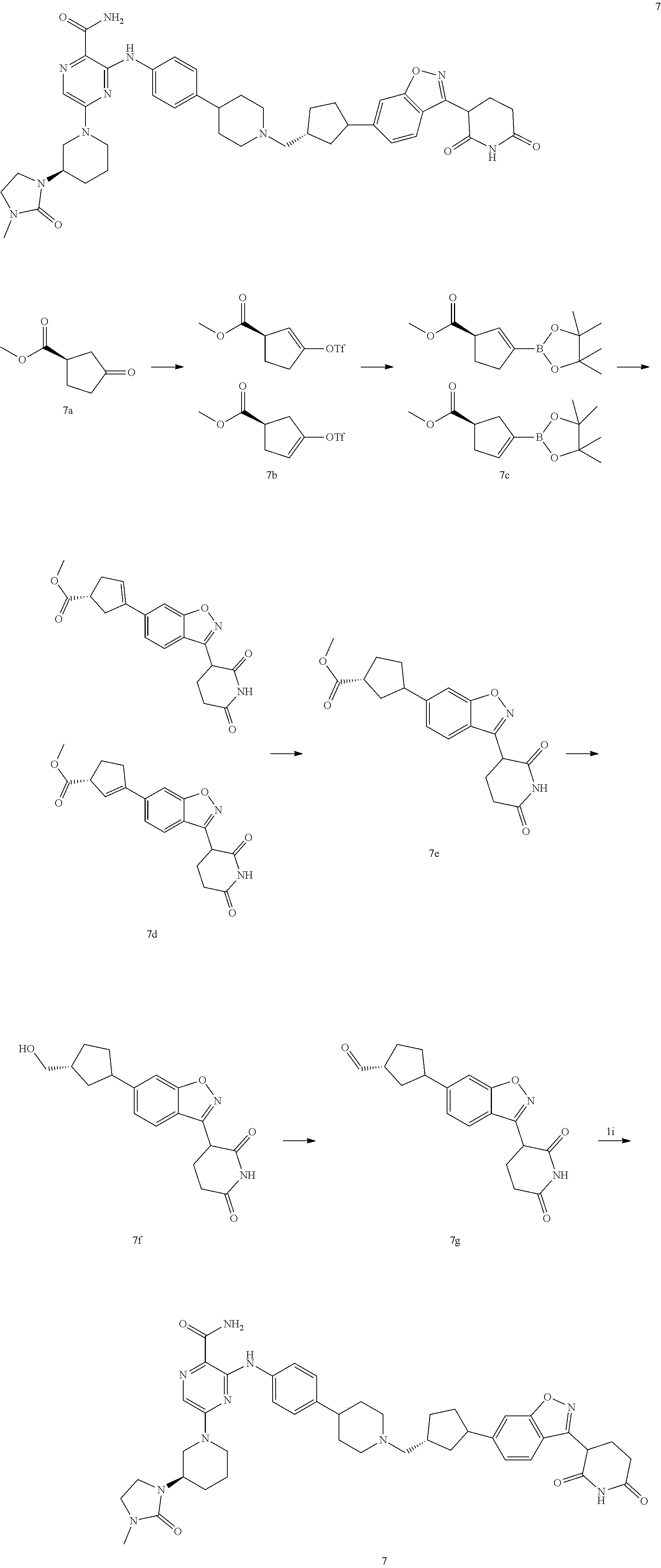

### Step 1: Preparation of Compound 7

Referring to the procedures of the first step to the eighth step in Example 6, Example 7 was synthesized, except that intermediate 6a was replaced by intermediate 7a as a starting material.

MS (ESI, [M+H]⁺) m/z: 789.5.

¹H NMR (500 MHz, DMSO-d₆) δ 11.20 (s, 1H), 11.09 (s, 1H), 7.78-7.72 (m, 2H), 7.64 (d, J=16.6 Hz, 2H), 7.50 (d, J=8.1 Hz, 2H), 7.36-7.29 (m, 2H), 7.16 (d, J=8.1 Hz, 2H), 4.57 (dd, J=11.9, 5.0 Hz, 1H), 4.36 (d, J=12.2 Hz, 1H), 4.28 (d, J=13.1 Hz, 1H), 3.60 (ddt, J=12.0, 5.3, 2.9 Hz, 1H), 3.30 (s, 2H), 3.27-3.19 (mi, 3H), 2.99 (dt, J=37.1, 11.7 Hz, 4H), 2.78 (ddd, J=17.2, 12.0, 5.3 Hz, 1H), 2.70 (d, J=2.4 Hz, 3H), 2.61 (dt, J=17.3, 4.3 Hz, 1H), 2.49-2.44 (mi, 1H), 2.19 (dq, J=13.8, 5.0 Hz, 2H), 2.08 (d, J=8.6 Hz, 2H), 2.00 (d, J=7.5 Hz, 1H), 1.91 (s, 1H), 1.86-1.62 (m, 8H), 1.56 (d, J=13.2 Hz, 2H).

### Example 38: Preparation of Compound 8

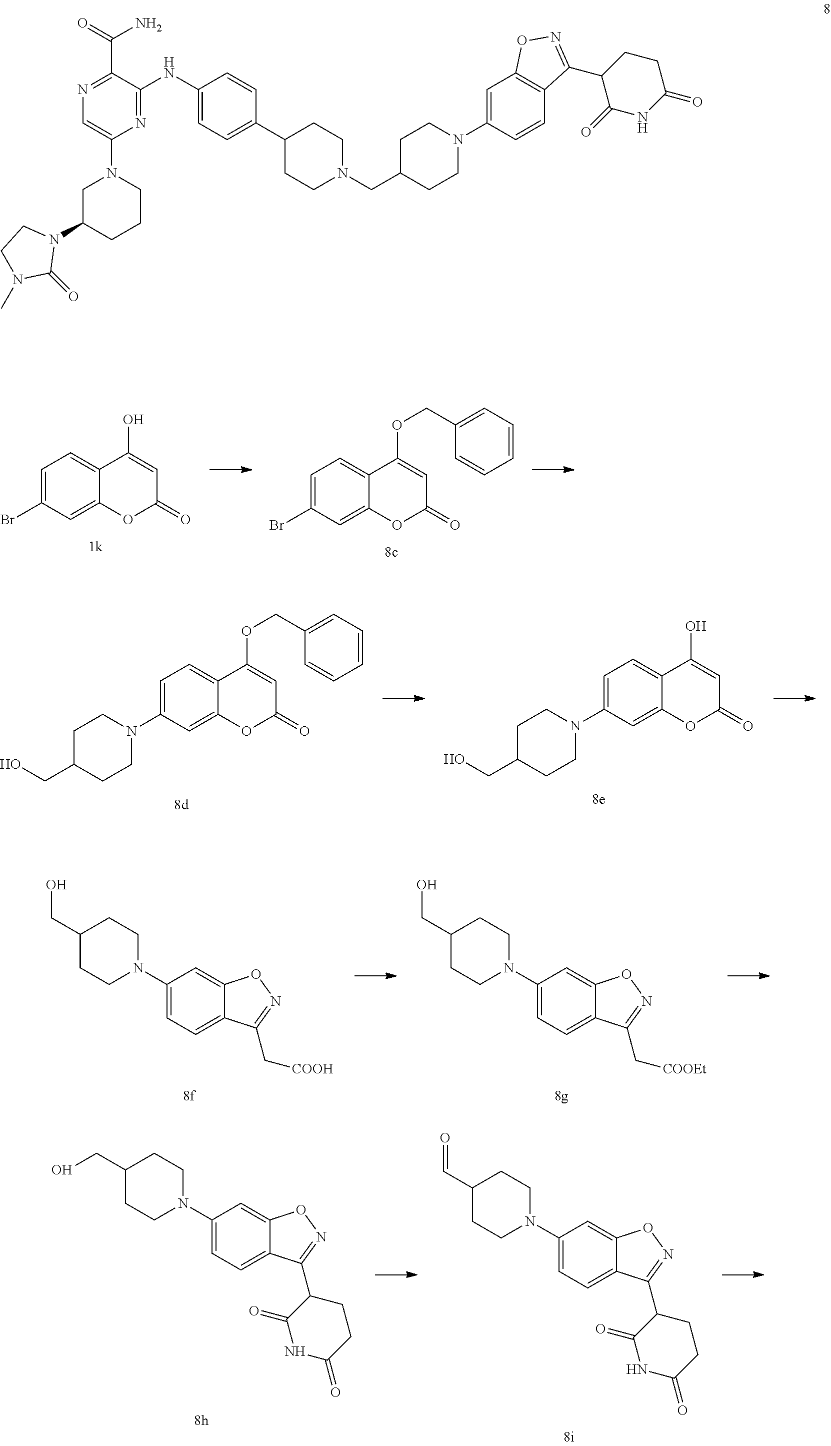

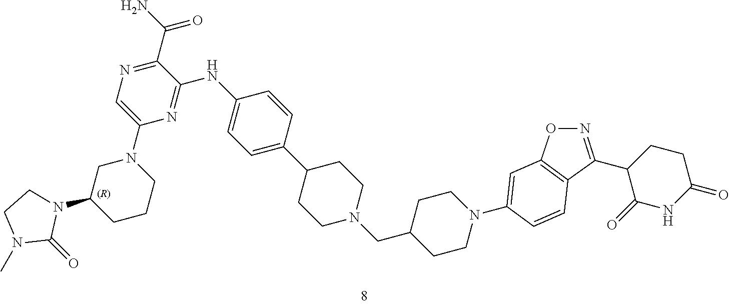

### Step 1: Preparation of Intermediate 8c

Intermediate 8b (27 g), benzyl bromide (36.8 g), potassium carbonate (29.8 g), and 700 mL of acetonitrile were added to a single-necked flask in sequence, and the mixture was heated to 80° C. and reacted for 5 h. After the reaction solution was concentrated by evaporation at reduced pressure to remove the solvent, the crude product was separated by silica gel column chromatography (eluent: EA) to give 2.06 g of intermediate 8c as the target product.

MS(ESI, [M+H]⁺) m/z: 331.03.

¹H NMR (500 MHz, DMSO-d₆) δ 7.76-7.70 (m, 2H), 7.54 (dt, J=7.8, 1.8 Hz, 3H), 7.49-7.43 (m, 2H), 7.43-7.38 (m, 1H), 6.09 (s, 1H), 5.35 (s, 2H).

### Step 2: Preparation of Intermediate 8d

Intermediate 8c (4 g), 4-hydroxymethylpiperidine (2.77 g), cesium carbonate (7.82 g), palladium chloride (0.213 g), and 1,4-dioxane (150 mL) were added to a single-necked flask in sequence, and the mixture was reacted at 80° C. for 16 h under N₂ atmosphere. The reaction solution was cooled to room temperature and concentrated by evaporation at reduced pressure to remove the solvent, and the crude product was separated and purified by silica gel column chromatography (eluent: PE:EA=1:1, v/v) to give 4.82 g of intermediate cd.

MS(ESI, [M+H]⁺) m/z: 366.

¹H NMR (500 MHz, DMSO-d₆) δ 7.55 (d, J=9.0 Hz, 1H), 7.53-7.47 (m, 3H), 7.46-7.42 (m, 2H), 7.40 (dd, J=3.8, 2.0 Hz, 1H), 6.93 (dd, J=9.1, 2.5 Hz, 1H), 5.70 (s, 1H), 5.30 (s, 2H), 4.49 (t, J=5.3 Hz, 1H), 3.93 (dt, J=13.3, 3.5 Hz, 2H), 3.27 (t, J=5.8 Hz, 2H), 2.84 (td, J=12.7, 2.7 Hz, 2H), 1.76-1.69 (m, 2H), 1.65-1.58 (m, 1H), 1.21-1.15 (m, 2H).

### Step 3: Preparation of Intermediate 8e

Intermediate 8d (4.00 g), palladium on carbon (2.0 g), and methanol (300 mL) were added to a single-necked flask in sequence. After the mixture was purged several times with H₂, the mixture was reacted at room temperature for 16 h. The mixture was filtered under vacuum, the filtrate was concentrated by evaporation at reduced pressure to remove the solvent, and the crude product was separated by silica gel column chromatography (PE:EA=2:1, v/v) to give 1.70 g of intermediate 8e.

MS(ESI, [M+H]⁺) m/z: 276.1

¹H NMR (500 MHz, DMSO-d₆) δ 12.01 (s, 1H), 7.56 (d, J=8.9 Hz, 1H), 6.92 (dd, J=9.0, 2.4 Hz, 1H), 6.73 (d, J=2.4 Hz, 1H), 5.31 (s, 1H), 4.46 (s, 1H), 3.93 (dt, J=13.2, 3.2 Hz, 2H), 3.27 (d, J=6.2 Hz, 2H), 22.84 (td, J=12.7, 2.7 Hz, 2H), 1.76-1.69 (m, 2H), 1.66-1.57 (m, 1H), 1.18 (qd, J=12.4, 4.1 Hz, 2H).

### Step 4: Preparation of Intermediate 8f

Intermediate 3e (1.9 g), hydroxylamine hydrochloride (1.555 g), sodium ethoxide (1.523 g), and ethanol (150 mL) were added to a single-necked flask in sequence, and the mixture was reacted at 85° C. for 22 h. The reaction solution was cooled to room temperature and concentrated by evaporation at reduced pressure to remove the solvent. Dichloromethane and a saturated aqueous sodium carbonate solution were added to the crude product. The mixture was extracted and separated, the organic phase was discarded, and the pH of the aqueous phase was adjusted with 6 M HCl to 6. The mixture was extracted several times with dichloromethane. The organic phases were combined, washed with saturated brine, dried over anhydrous sodium sulfate, and filtered. The filtrate was concentrated by evaporation at reduced pressure to remove the solvent, thus giving 1.32 g of intermediate 8f.

MS(ESI, [M+H]⁻) m/z: 289.1.

¹H NMR (500 MHz, DMSO-d₆) δ 12.74 (s, 1H), 7.53 (d, J=8.9 Hz, 1H), 7.07 (dd, J=8.9, 2.1 Hz, 1H), 7.01 (d, J=2.0 Hz, 1H), 4.49 (t, J=5.4 Hz, 1H), 3.94-3.87 (m, 4H), 3.34 (s, 2H), 2.80 (td, J=12.5, 2.6 Hz, 2H), 1.77-1.71 (m, 2H), 1.63-1.57 (m, 1H), 1.22 (dd, J=12.4, 3.7 Hz, 2H).

### Step 5: Preparation of Intermediate 8g

Intermediate 8f (1.3 g), ethanol (100 mL), and sulfuric acid (1.471 g, 15.00 mmol) were added to a single-necked flask in sequence, and the mixture was reacted at 85° C. for 5 h. The reaction solution was cooled to room temperature, and dichloromethane and a saturated aqueous sodium bicarbonate solution were added to the reaction solution to adjust the pH to 8. The organic phase was separated, and the aqueous phase was extracted with dichloromethane. The organic phases were combined, washed with saturated brine, dried over anhydrous sodium sulfate, and filtered. The filtrate was concentrated by evaporation at reduced pressure to remove the solvent, and the crude product was purified by silica gel column chromatography (PE:EA=1:4, v/v) to give 1.01 g of intermediate 8g.

MS(ESI, [M+H]⁺) m/z: 319.16.

¹H NMR (500 MHz, DMSO-d₆) δ 7.53 (d, J=9.0 Hz, 1H), 7.08 (dd, J=9.0, 2.1 Hz, 1H), 7.02 (d, J=2.1 Hz, 1H), 4.49 (t, J=5.3 Hz, 1H), 4.13 (q, J=7.1 Hz, 2H), 4.04 (s, 2H), 3.89 (dt, J=12.2, 3.4 Hz, 2H), 3.28 (t, J=5.8 Hz, 2H), 3.17 (d, J=5.2 Hz, 1H), 2.81 (td, J=12.6, 2.7 Hz, 2H), 1.74 (dd, J=13.7, 3.7 Hz, 2H), 1.26-1.17 (m, 5H).

### Step 6: Preparation of Intermediate 8h

Acrylamide (48.7 mg) was slowly added dropwise to a stirred solution of intermediate 8g (200 mg) in THF (50 mL) at 0° C. under N₂ atmosphere. After the dropwise addition was carried out for 1 min and completed, a solution of potassium tert-butoxide in THF (1 M) (0.935 mL) was added dropwise, and after the dropwise addition was carried out for about 1 min and completed, the mixture was stirred and reacted at 0° C. for 3 h. The reaction solution was poured into an aqueous ammonium chloride solution. After the mixture was vigorously stirred for 1 min, the mixture was extracted with EA, washed with saturated brine, dried over anhydrous sodium sulfate, and filtered. The filtrate was concentrated by evaporation at reduced pressure to remove the solvent, and the crude product was separated by silica gel column chromatography (PE:EA=1:4, v/v) to give 0.06 g of intermediate 8h.

MS(ESI, [M+H]⁺) m/z: 344.20.

### Step 7: Preparation of Intermediate 81

Intermediate 8h (45 mg), IBX oxidant (108 mg), and acetonitrile (10 mL) were added to a single-necked flask in sequence, and the mixture was reacted at 85° C. for 0.5 h. The reaction solution was cooled to room temperature and filtered, and the filtrate (containing intermediate 8i) was directly used in the next step. MS(ESI, [M+H]⁺) m/z: 342.16.

### Step 8: Preparation of Compound 8

The intermediate 8i reaction solution (the solution of intermediate 8i obtained in the previous step) and MeOH (10.00 mL) were added to a single-necked flask. Intermediate 1i (68.7 mg) and acetic acid (3.85 mg) were added in sequence with stirring at room temperature. After the mixture was stirred at room temperature for 30 min, sodium cyanoborohydride (16.11 mg) was added, and the mixture was reacted at room temperature for 21 h. The reaction solution was poured into a mixed solution of dichloromethane and water, and the pH was adjusted to 8 with saturated sodium bicarbonate. The organic phase was separated, and the aqueous phase was extracted several times with dichloromethane. The organic phases were combined, washed with saturated brine, dried over anhydrous sodium sulfate, and filtered. The filtrate was concentrated by evaporation at reduced pressure to remove the solvent, and the crude product was dissolved in DMSO, purified by C₁₈ reversed-phase column, and purified by Biotage medium and low pressure chromatography (1 mM aqueous ammonium acetate solution:acetonitrile=1:1, v/v) to give 27.6 mg of compound 8.

HR-MS(ESI, [M+H]⁺) m/z: 804.43146.

¹H NMR (500 MHz, DMSO-d₆) δ 11.21 (s, 1H), 11.05 (s, 1H), 7.79-7.73 (m, 1H), 7.66 (s, 1H), 7.53 (dd, J=22.1, 8.4 Hz, 3H), 7.36-7.32 (m, 1H), 7.17 (d, J=8.1 Hz, 2H), 7.09-7.04 (m, 2H), 4.45 (dd, J=11.4, 5.0 Hz, 1H), 4.39-4.32 (m, 1H), 4.29 (d, J=13.1 Hz, 1H), 3.90 (d, J=12.3 Hz, 2H), 3.62 (dq, J=11.2, 6.6, 5.5 Hz, 1H), 3.26 (dd, J=11.5, 5.1 Hz, 4H), 3.06-2.81 (m, 6H), 2.61-2.56 (m, 1H), 2.54-2.36 (m, 5H), 2.18 (dt, J=13.2, 5.0 Hz, 2H), 1.99-1.68 (m, 1H), 1.64-1.51 (m, 2H), 1.24 (q, J=11.4, 9.3 Hz, 4H).

### Example 9: Synthesis of Compound 9

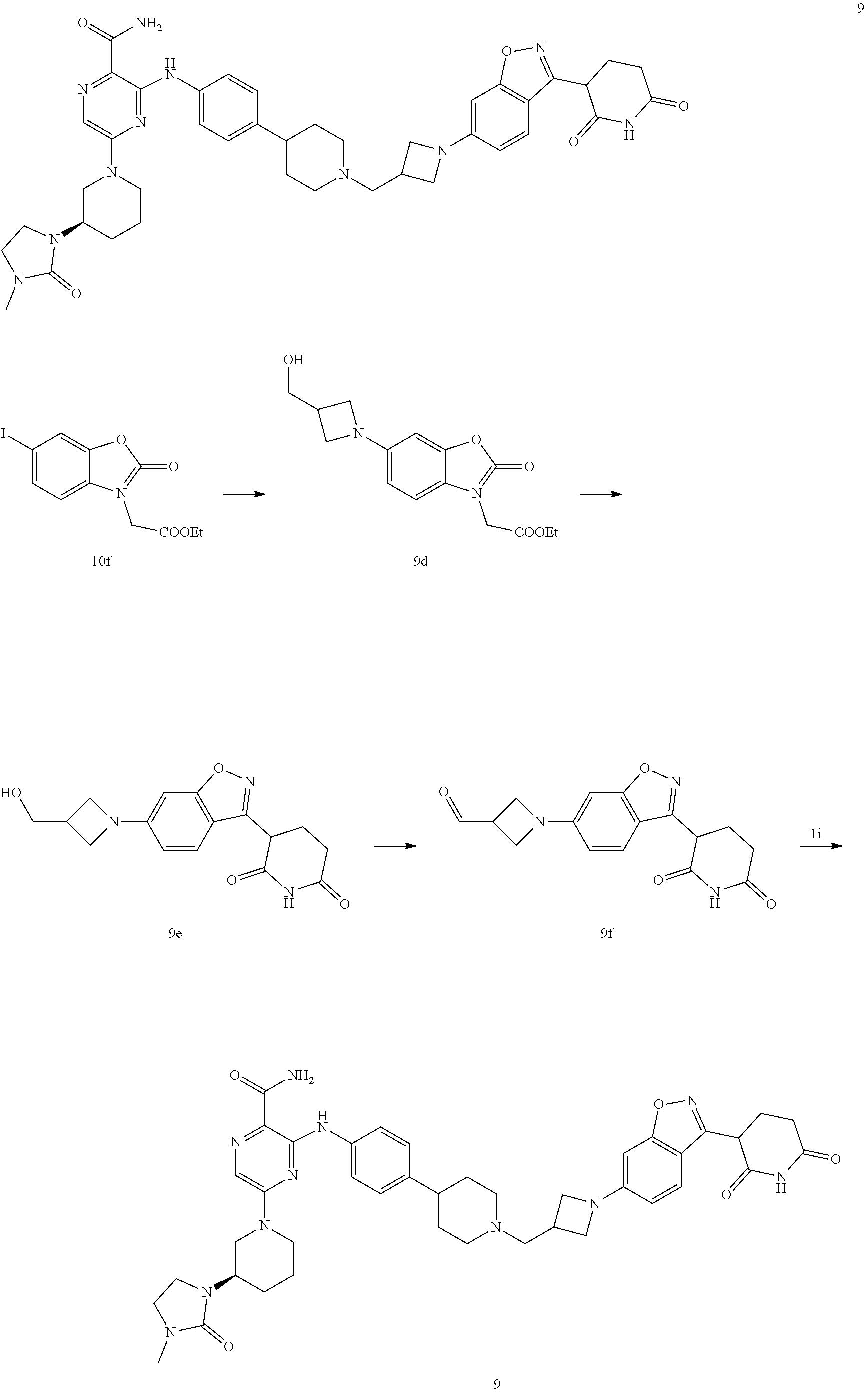

### Step 1: Preparation of Intermediate 9d

Intermediate 10f (10 g), azetidin-3-ylmethanol (3.51 g), L-proline (1.547 g), copper(I) iodide (1.280 g), DMF (100 mL), and sodium carbonate (8.55 g) were added to a reaction flask in sequence, and the mixture was heated to 100° C. and reacted for 4 h under N₂ atmosphere. The reaction was stopped, and the reaction solution was cooled to room temperature and extracted three times with the organic solvent DCM (200 mL) and water (500 mL). The organic phase was separated, washed with 500 mL of water, washed with 500 mL of saturated brine, dried over anhydrous sodium sulfate, and filtered. The filtrate was purified by silica gel column chromatography (eluent: EA) to give 2.16 g of intermediate 9d.

MS(ESI, [M+H]⁺) m/z: 291.2.

¹H NMR (500 MHz, DMSO-d₆) δ 7.45-7.38 (m, 1H), 7.36 (ddt, J=6.6, 4.9, 2.6 Hz, 1H), 6.37 (d, J=7.9 Hz, 1H), 4.73 (t, J=5.3 Hz, 1H), 4.02 (q, J=7.1 Hz, 2H), 3.91 (s, 1H), 3.83 (t, J=7.9 Hz, 1H), 3.55 (dd, J=7.8, 5.4 Hz, 2H), 3.52-3.46 (m, 2H), 2.71 (ttd, J=12.4, 8.6, 7.2, 4.3 Hz, 1H), 1.08 (t, J=7.1 Hz, 3H).

### Step 2: Preparation of Intermediate 9e

Intermediate 9d (200 mg) and THF (10 mL) were added to a reaction flask in sequence. The mixture was cooled to about 0° C., acrylamide (53.9 mg) and potassium tert-butoxide (116 mg) were added, and the mixture was reacted at 0° C. for 3.5 h under N₂ atmosphere. The reaction was stopped, and the reaction solution was added dropwise to a saturated aqueous ammonium chloride solution. The mixture was extracted with 50 mL of ethyl acetate, the organic phase was separated, washed with 50 mL of saturated brine, dried over anhydrous sodium sulfate and filtered, and the filtrate was purified by silica gel column chromatography (eluent EA) to give 0.035 g of intermediate 9e.

MS(ESI, [M+H]⁺) m/z: 316.1.

¹H NMR (500 MHz, DMSO-d₆) δ 11.03 (s, 1H), 7.53 (d, J=8.6 Hz, 1H), 6.49 (d, J=1.8 Hz, 1H), 6.46 (dd, J=8.6, 1.9 Hz, 1H), 4.80 (t, J=5.2 Hz, 1H), 4.42 (dd, J=11.3, 5.0 Hz, 1H), 3.94 (t, J=7.8 Hz, 2H), 3.65 (dd, J=7.7, 5.4 Hz, 2H), 3.59 (t, J=5.8 Hz, 2H), 2.82 (tdd, J=8.2, 6.8, 6.0, 3.6 Hz, 1H), 2.73 (ddd, J=17.0, 11.5, 5.3 Hz, 1H), 2.58 (dt, J=17.2, 4.4 Hz, 1H), 2.41 (dtd, J=13.1, 11.4, 4.6 Hz, 1H), 2.16 (dq, J=13.3, 5.0 Hz, 1H).

### Step 3: Preparation of Example 9

Intermediate 9e (200 mg), acetonitrile (10.00 mL), and IBX oxidant (533 mg) were added to a reaction flask in sequence, and the mixture was reacted at 80° C. for 1 h. The reaction solution was filtered and evaporated to remove a small amount of the solvent (containing intermediate 9f). MeOH (10 mL), intermediate 1i (304 mg), and glacial acetic acid (19.04 mg) were added. After the mixture was stirred at room temperature for 30 min, sodium cyanoborohydride (80 mg) was added, and the mixture was reacted at room temperature for 3 h. The mixture was purified by silica gel column chromatography (DCM:CH₃OH=10:1, v/v). The mixture was then purified by 120 g C₁₈ reversed-phase column (10 mM aqueous ammonium acetate solution:CH₃CN=40%:60%) to give 0.09 g of compound 9.

MS(ESI, [M+H]⁺) m/z: 776.5.

¹H NMR (500 MHz, DMSO-d₆) δ 11.20 (s, 1H), 11.04 (s, 1H), 7.76 (d, J=2.9 Hz, 1H), 7.66 (s, 1H), 7.52 (dd, J=22.5, 8.4 Hz, 3H), 7.33 (d, J=2.9 Hz, 1H), 7.16 (d, J=8.2 Hz, 2H), 6.53-6.45 (m, 2H), 4.43 (dd, J=11.4, 5.0 Hz, 1H), 4.35 (d, J=12.2 Hz, 1H), 4.29 (d, J=13.4 Hz, 1H), 4.04 (t, J=7.7 Hz, 2H), 3.60 (ddd, J=13.0, 8.6, 5.4 Hz, 3H), 3.32-3.23 (m, 4H), 3.04 (d, J=11.8 Hz, 1H), 3.01-2.91 (m, 4H), 2.72 (s, 4H), 2.64-2.52 (m, 3H), 2.48-2.36 (m, 2H), 2.17 (dq, J=13.2, 5.0 Hz, 1H), 2.05 (t, J=11.6 Hz, 2H), 1.87-1.80 (m, 2H), 1.80-1.70 (m, 3H), 1.67-1.52 (m, 3H).

¹³C NMR (126 MHz, DMSO-d₆) δ 173.60, 172.48, 172.09, 169.70, 165.08, 160.79, 156.41, 154.04, 154.02, 150.98, 140.26, 137.83, 127.44, 122.90, 120.26, 118.66, 114.71, 111.21, 110.23, 89.41, 56.68, 55.38, 54.31, 49.12, 47.24, 45.46, 44.85, 41.51, 39.03, 33.59, 31.56, 31.40, 28.21, 27.80, 24.45, 23.29, 21.54.

### Example 10: Synthesis of Compound 10

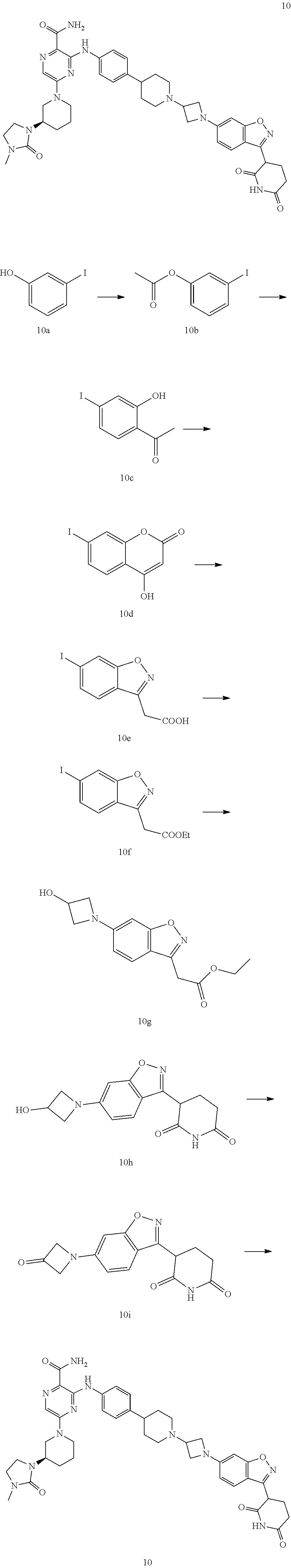

### Step 1: Preparation of Intermediate 10b

Intermediate 10a (100 g), triethylamine (92 g), and DCM (1 L) were added to a single-necked flask in sequence, acetyl chloride (39.2 g) was added at 0° C., and the mixture was reacted at room temperature for 2.5 h. The system was concentrated to remove DCM, 300 mL×3 of petroleum ether was added to the system, and the mixture was extracted with 1000 mL of saturated brine. The organic phase was separated, dried over anhydrous sodium sulfate, filtered, and concentrated to give 123 g of intermediate 10b.

MS(ESI, [M+H]⁺) m/z: 263.0.

### Step 2: Preparation of Intermediate 10c

Intermediate 10b (123 g) and anhydrous aluminum trichloride (94 g) were added to a single-necked flask in sequence, and the mixture was heated to 170° C. and reacted for 3 h. The reaction solution was cooled to room temperature. About 500 mL of 6 M hydrochloric acid was slowly added to quench the reaction. After a solid present therein was crushed, the mixture was extracted with DCM and filtered. Liquid separation was performed, the organic phase was collected, and the aqueous phase was then extracted 2 times with 250 mL of dichloromethane. The organic phases were combined, dried over anhydrous sodium sulfate, filtered and concentrated by evaporation at reduced pressure to remove the solvent, thus giving 112 g of intermediate 10c.

MS(ESI, [M+H]⁺) m/z: 263.1.

### Step 3: Preparation of Intermediate 10d

Intermediate 10c (107 g), diethyl carbonate (174 g), and toluene (1000 mL) were added to a three-necked flask in sequence. After dissolution, the mixture was cooled to about 0° C. Sodium hydride (58.8 g) was added in portions, and the mixture was warmed to 100° C. The mixture was warmed to 120° C. and reacted for about 1.5 h after the system was stable. The reaction solution was cooled to room temperature and slowly poured into 2 L of stirred ice water. The mixture was extracted with 500 mL of ethyl acetate, and the organic phase was discarded. The aqueous phase was adjusted to pH=1-2 with 3 N hydrochloric acid and filtered to give 90 g of intermediate 10d.

MS(ESI, [M+H]⁺) m/z: 289.1.

### Step 4: Preparation of Intermediate 10e

Intermediate 10d (90 g), hydroxylamine hydrochloride (43.4 g), and absolute ethanol (1000 mL) were added to a single-necked flask in sequence at 0° C. After dissolution, sodium ethoxide (42.5 g) was added in portions under N₂ atmosphere, and the mixture was heated to 90° C. and reacted for 4.5 h. The reaction solution was cooled to room temperature, and 3 N hydrochloric acid was added to adjust the pH to 1-3. The mixture was concentrated by evaporation at reduced pressure to remove the solvent, 500 mL of water was added to the residue, and the mixture was stirred at room temperature for 30 min and filtered. The filter cake was washed with 200 mL of water and then transferred to a reduced-pressure oven to be dried, thus giving 91.6 g of intermediate 10e.

MS(ESI, [M+H]⁺) m/z: 304.1.

¹H NMR (500 MHz, DMSO-d₆) δ 8.22 (d, J=1.2 Hz, 1H), 7.71 (dd, J=8.3, 1.3 Hz, 1H), 7.64 (d, J=8.3 Hz, 1H), 4.00 (s, 2H).

### Step 5: Preparation of Intermediate 10f

Intermediate 10e (91.6 g) and absolute ethanol (1000 mL) were added to a single-necked flask in sequence, concentrated sulfuric acid (148 g, 1511 mmol) was added dropwise at 0° C. over about 3 min (the system was about 50° C.), and the mixture was heated to 85° C. and reacted for 3.5 h under N₂ atmosphere. The reaction solution was cooled to room temperature with stirring. The solvent was removed from the reaction solution, 1500 mL of ice water and 1000 mL of ethyl acetate were added to the residue, a 10% aqueous NaOH solution was added dropwise under an ice-water bath, and the pH was adjusted to about 9 (internal temperature<5° C.). The mixture was extracted and separated three times by a separating funnel, and the organic phases were combined, washed with saturated brine, dried over anhydrous sodium sulfate, and subjected to rotary evaporation to remove the solvent, thus giving 102.6 g of intermediate 10f.

MS(ESI, [M+H]⁺) m/z: 332.0.

### Step 6: Preparation of Intermediate 10g

Intermediate 10f (30 g), azetidin-3-ol (7.29 g), L-proline (5.22 g), copper(I) iodide (4.31 g), anhydrous sodium carbonate (28.8 g), and DMF (300 mL) were added to a single-necked flask in sequence, and the mixture was heated to 100° C. and reacted for 2.5 h under N₂ atmosphere. The reaction solution was cooled to room temperature and extracted three times with 200 mL×3 of the organic solvent ethyl acetate and 1000 mL of water. After the organic phases were separated and combined, the organic phase was washed with 500 mL of water, washed with 500 mL of saturated brine, dried over anhydrous sodium sulfate, and filtered. The filtrate was concentrated by evaporation at reduced pressure to remove the solvent, thus giving a crude product, which was purified by silica gel column chromatography to give 12 g of intermediate 10g.

MS(ESI, [M−H]⁻) m/z: 275.1.

### Step 7: Preparation of Intermediate 10h

Intermediate 10g (10 g) and anhydrous tetrahydrofuran (200 mL) were added to a three-necked flask in sequence. After dissolution, acrylamide (1.976 g) was added. Under N₂ atmosphere, the mixture was cooled to about −15° C., potassium tert-butoxide (3.894 g) was added dropwise, and the system was kept at −15° C. and reacted for about 1.5 h. The reaction solution was added dropwise to a saturated ammonium chloride solution to quench the reaction, and the mixture was extracted with 100 mL×3 of ethyl acetate. The organic phase was separated, washed with 100 mL of saturated brine, dried over anhydrous sodium sulfate, concentrated by evaporation at reduced pressure to remove the solvent, and purified by silica gel column chromatography to give 6.3 g of intermediate 10h.

MS(ESI, [M+H]⁺) m/z: 302.1.

### Step 8: Preparation of Intermediate 101

At room temperature, 2-iodoxybenzoic acid (IBX, 9.32 g) was added to a stirred solution of intermediate 10h in anhydrous acetonitrile (50 mL), and the mixture was reacted at 90° C. for about 3.5 h and filtered. After the filter cake was washed twice with acetonitrile, the filtrates were combined, concentrated by evaporation at reduced pressure to remove the solvent, and purified by silica gel column chromatography to give 4.6 g of intermediate 101.

MS(ESI, [M+H]⁺) m/z: 300.1.

### Step 9: Preparation of Compound 10

Intermediate 1i (150 mg), intermediate 10i (133 mg), dichloromethane (10 mL), and glacial acetic acid (50.4 mg, 0.048 mL) were first added to a reaction flask. After the mixture was stirred at room temperature for 30 min, sodium cyanoborohydride (70.3 mg) was added, and the mixture was stirred at room temperature overnight. After the reaction was completed, 20 mL of a saturated aqueous NaHCO₃ solution was added to the reaction solution, and the mixture was extracted twice with 20 mL of DCM-MeOH (10:1). The extracts were combined, dried over anhydrous sodium sulfate, and filtered under vacuum, and the resulting filtrate was subjected to rotary evaporation to dryness and purified by C₁₈ reversed-phase column (10 nM aqueous ammonium acetate solution-acetonitrile=50%:50%, v/v) to give 59 mg of compound 10.

Q-TOF (ESI, [M+H]⁺) m/z: 762.3847.

¹H NMR (500 MHz, DMSO-d₆) δ 11.20 (s, 1H), 11.04 (s, 1H), 7.76 (d, J=2.5 Hz, 1H), 7.65 (s, 1H), 7.56 (d, J=8.6 Hz, 1H), 7.49 (d, J=8.3 Hz, 2H), 7.33 (d, J=2.8 Hz, 1H), 7.16 (d, J=8.2 Hz, 2H), 6.56-6.47 (m, 2H), 4.44 (dd, J=11.4, 5.0 Hz, 1H), 4.34 (d, J=12.6 Hz, 1H), 4.28 (d, J=13.2 Hz, 1H), 4.04 (t, J=7.3 Hz, 2H), 3.72 (dd, J=8.0, 5.3 Hz, 2H), 3.61 (td, J=11.8, 10.6, 5.1 Hz, 1H), 3.32-3.20 (m, 4H), 3.03 (t, J=11.8 Hz, 1H), 2.99-2.90 (m, 3H), 2.70 (s, 4H), 2.59 (dt, J=17.4, 4.5 Hz, 1H), 2.49-2.37 (m, 2H), 2.17 (dq, J=14.1, 4.9 Hz, 1H), 1.96 (t, J=11.2 Hz, 2H), 1.85-1.69 (m, 5H), 1.59 (dq, J=15.5, 12.0 Hz, 3H).

¹³C NMR (126 MHz, DMSO-d₆) δ 173.61, 172.09, 169.70, 165.06, 160.78, 156.44, 154.04, 153.82, 150.97, 140.19, 137.84, 127.43, 122.97, 120.26, 118.67, 114.71, 111.34, 110.33, 89.58, 56.40, 54.97, 50.51, 49.13, 47.25, 45.44, 44.85, 41.48, 33.25, 31.55, 31.41, 28.19, 24.44, 23.29.

### Example 11: Synthesis of Compound 11

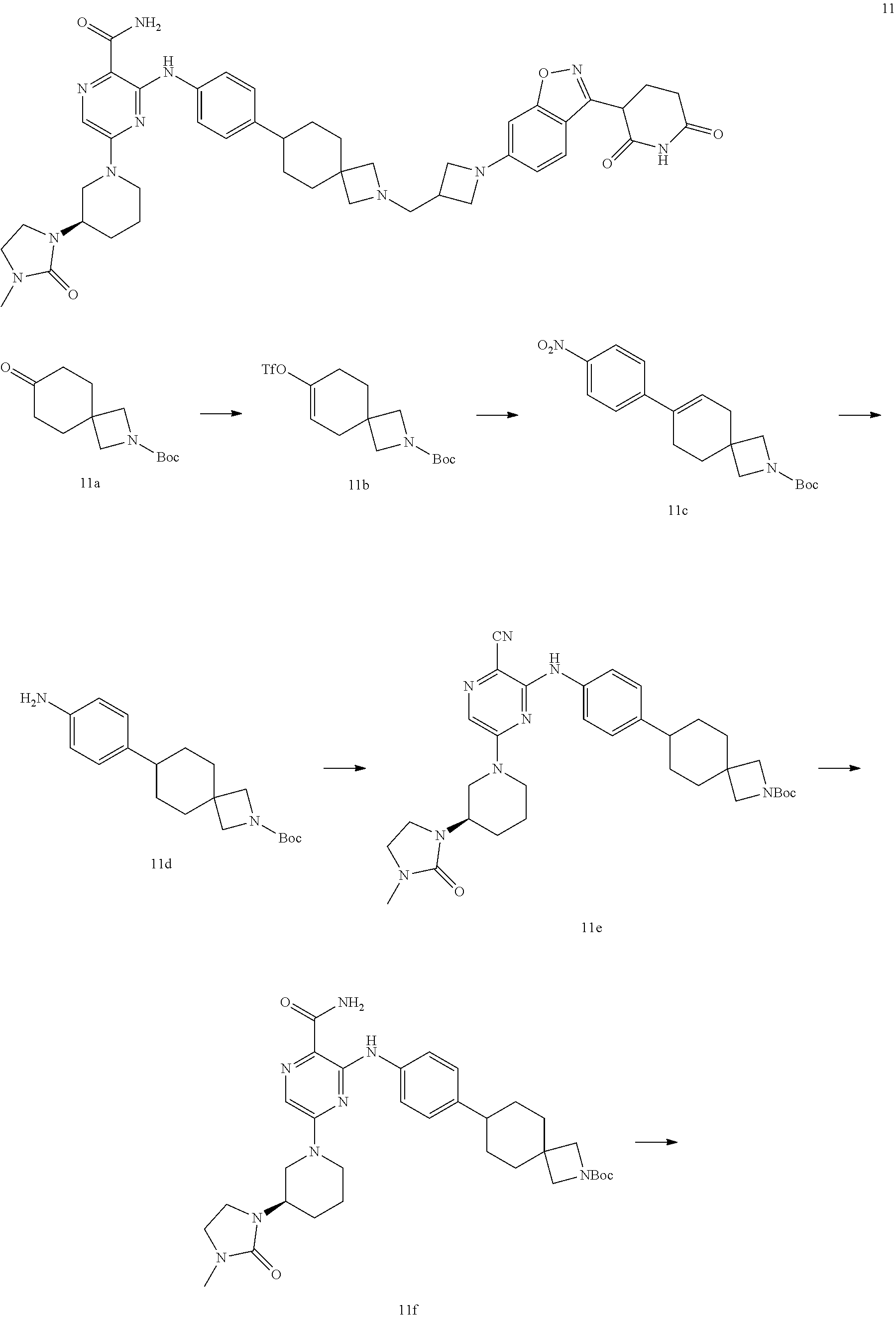

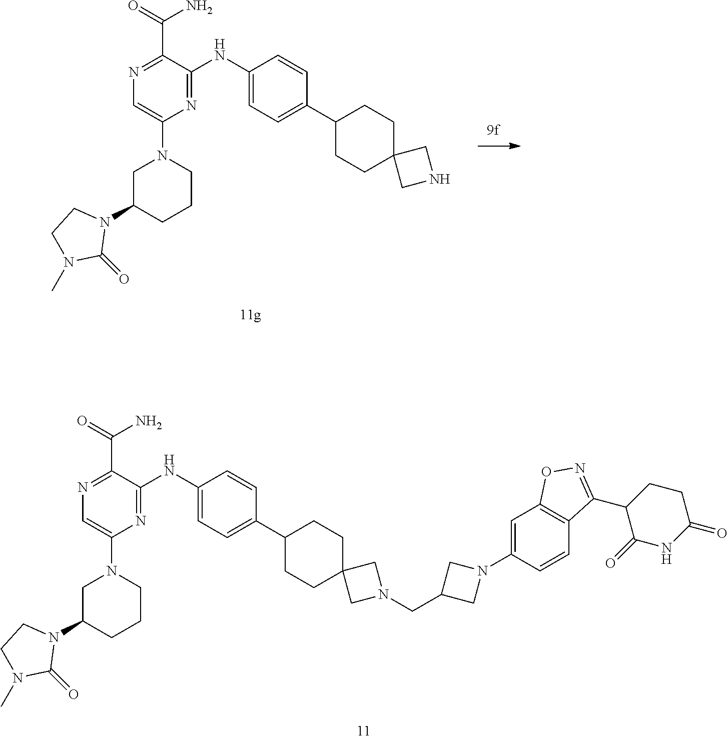 [SMILES: CN1CCN([C@@H]2CCCN(c3cnc(C(N)=O)c(Nc4ccc(C5CCC6(CC5)CNC6)cc4)n3)C2)C1=O ; CN1CCN([C@@H]2CCCN(c3cnc(C(N)=O)c(Nc4ccc(C5CCC6(CC5)CN(CC5CN(c7ccc8c(C9CCC(=O)NC9=O)noc8c7)C5)C6)cc4)n3)C2)C1=O]

### Step 1: Preparation of Intermediate 11b

Lithium bis(trimethylsilyl)amide (9.44 g) was slowly added dropwise to a stirred solution of intermediate 11a (9 g) in THF (150 mL) at −78° C. under N₂ atmosphere. After the dropwise addition was carried out for 5 min and completed, the mixture was stirred and reacted at −78° C. for 0.5 h. A solution of N-phenylbis(trifluoromethanesulfonyl)imide (17.47 g) in THF (150 mL) was slowly added to the reaction solution, and the temperature was controlled below −60° C. After the addition was completed, the mixture was reacted at −78° C. for 3.5 h. After the reaction was completed, the reaction solution was poured into a saturated ammonium chloride crushed ice solution, and the mixture was extracted twice with 200 mL of EA. The organic phases were combined, washed once with saturated brine, dried over anhydrous sodium sulfate, filtered, concentrated, and purified by silica gel column chromatography (PE:EA=3:2, v/v) to give 13 g of intermediate 11b.

¹H NMR (500 MHz, DMSO-d₆) δ 5.87-5.77 (m, 1H), 3.57 (d, J=51.4 Hz, 4H), 2.40 (d, J=4.3 Hz, 4H), 1.90 (t, J=6.3 Hz, 2H), 1.38 (s, 9H).

### Step 2: Preparation of Intermediate 11c

4-Nitrophenylboronic acid pinacol ester (10.06 g), intermediate 11b (10 g), potassium carbonate (11.16 g), [1,1′-bis(diphenylphosphino)ferrocene]palladium dichloride dichloromethane complex (4.40 g), 1,4-dioxane (300 mL), and water (50 mL) were added to a single-necked flask in sequence. After the mixture was purged several times with N₂, the mixture was heated to 90° C. and reacted for 5 h. After the reaction was completed, the reaction solution was filtered, the filter cake was washed several times with EA, and 500 mL of EA and 200 mL of water were added to the filtrate. The organic phase was separated, washed with 500 mL of saturated brine, dried over anhydrous sodium sulfate, and filtered. The filtrate was purified by silica gel column chromatography (PE:EA=4:1, v/v). 5.58 g of intermediate 11c was obtained.

MS(ESI, [M+H]⁺) m/z: 345.05

¹H NMR (500 MHz, DMSO-d₆) δ 8.26-8.15 (m, 2H), 7.75-7.61 (m, 2H), 6.43-6.33 (m, 1H), 3.60 (d, J=49.0 Hz, 4H), 2.52 (d, J=3.8 Hz, 2H), 2.46 (dt, J=4.5, 2.4 Hz, 2H), 1.89 (t, J=6.2 Hz, 2H), 1.38 (s, 9H).

### Step 3: Preparation of Intermediate 11d

Pd/C (10%, 0.015 g) was added to a solution of intermediate 11c (1 g) in MeOH (50 mL). The reaction solution was first purged 2-3 times with nitrogen, then purged 2-3 times with hydrogen, and stirred at room temperature and reacted for 90 min. After the reaction was completed, the reaction solution was filtered, and the filter cake was rinsed with 50 mL of DCM solvent. The filtrate was concentrated by evaporation at reduced pressure to remove the solvent, thus giving 1.013 g of intermediate 11d.

MS(ESI, [M+H]⁺) m/z: 317.2.

¹H NMR (500 MHz, DMSO-d₆) δ 6.87-6.79 (m, 2H), 6.51-6.42 (m, 2H), 4.79 (s, 2H), 3.58 (d, J=13.2 Hz, 2H), 3.47 (s, 2H), 2.24 (tt, J=11.9, 3.4 Hz, 1H), 1.87 (dd, J=12.9, 3.8 Hz, 2H), 1.66-1.57 (m, 2H), 1.48 (td, J=13.0, 3.5 Hz, 2H), 1.38 (s, 9H), 1.35-1.27 (m, 2H).

### Step 4: Preparation of Intermediate 11e

Intermediate 1f (1.1 g), intermediate 11d (1.053 g), BINAP (0.207 g), Cs₂CO₃ (3.25 g), Pd(OAc)₂ (0.075 g), and 1,4-dioxane (50 mL) were added to a single-necked flask in sequence, and the mixture was heated to 100° C. and reacted under N₂ atmosphere for 1.5 h. The reaction was stopped, the reaction solution was cooled to room temperature and filtered, and the filter cake was washed with 150 mL of dichloromethane. The filtrate was concentrated by evaporation at reduced pressure to remove the solvent and purified by silica gel column chromatography (DCM:CH₃OH=10:1). 1.2 g of intermediate 11e was obtained.

MS(ESI, [M+H]⁺) m/z: 601.6.

¹H NMR (500 MHz, DMSO-d₆) δ 8.93 (s, 1H), 7.81 (s, 1H), 7.47-7.41 (m, 2H), 7.10 (d, J=8.6 Hz, 2H), 4.24 (d, J=13.1 Hz, 1H), 3.66-3.43 (m, 6H), 3.25 (s, 4H), 3.00-2.86 (m, 2H), 2.71 (d, J=2.2 Hz, 3H), 2.37 (d, J=12.2 Hz, 1H), 1.91 (d, J=12.7 Hz, 2H), 1.82-1.65 (m, 6H), 1.53 (td, J=12.9, 3.5 Hz, 4H), 1.38 (s, 9H).

### Step 5: Preparation of Intermediate 11f

Intermediate 11e (1.2 g), DMSO (100 mL), MeOH (50 mL), and Cs₂CO₃ (0.532 g) were added to a single-necked flask in sequence. H₂O₂ (0.926 g) was added under an ice bath. After the mixture was stirred for 5 min, the ice bath was removed, and the mixture was reacted at room temperature for 1.5 h. The reaction was stopped, 100 mL of a saturated sodium sulfite solution was added to the reaction solution to quench the reaction. The color was not changed as detected using a potassium iodide starch reagent, and then 100 mL of ethyl acetate was added for extraction. The organic phases were combined, dried over anhydrous sodium sulfate, filtered and concentrated by evaporation at reduced pressure to remove the solvent, thus giving 1.2 g of intermediate Hf.

MS(ESI, [M+H]⁺) m/z: 619.41.

### Step 6: Preparation of Intermediate 11g

Intermediate 11f (1 g), DCM (20 mL), and trifluoroacetic acid (5.53 g, 3.74 mL) were added to a reaction flask in sequence, and the mixture was reacted at room temperature for 3 h. The solvent was removed by evaporation at reduced pressure to give a product of trifluoroacetate salt. A saturated aqueous sodium bicarbonate solution was slowly added. After the system was adjusted to weak alkalinity, the mixture was stirred for 1 h. The mixture was filtered, and the filter cake was washed with a small amount of water and dried in a vacuum drying oven to give 0.8 g of intermediate 11g.

MS(ESI, [M+H]⁺) m/z: 519.4.

¹H NMR (500 MHz, DMSO-d₆) δ 11.22 (s, 1H), 7.76 (d, J=2.7 Hz, 1H), 7.66 (s, 1H), 7.52-7.46 (m, 2H), 7.33 (d, J=2.8 Hz, 1H), 7.15-7.10 (m, 2H), 4.41-4.26 (m, 2H), 3.77 (s, 2H), 3.65 (s, 2H), 3.60 (dt, J=10.9, 4.0 Hz, 1H), 3.40-3.33 (m, 2H), 3.30-3.23 (m, 2H), 3.05 (t, J=11.8 Hz, 1H), 2.95 (t, J=12.2 Hz, 1H), 2.71 (s, 3H), 2.39 (tt, J=11.8, 3.0 Hz, 1H), 2.08 (d, J=12.8 Hz, 2H), 1.86-1.69 (m, 5H), 1.54 (td, J=13.3, 3.6 Hz, 3H), 1.38 (qd, J=13.1, 3.1 Hz, 2H).

### Step 11: Preparation of Compound 11

Intermediate 9f (50 mg), intermediate 11g (82 mg), MeOH (5 mL), and acetic acid (4.76 mg) were added to a reaction flask in sequence. After the mixture was stirred at room temperature for 30 min, sodium cyanoborohydride (19.93 mg) was added, and the mixture was stirred at room temperature for 3.5 h. The reaction solution was purified by silica gel column chromatography (DCM:CH₃OH=10:1) to give 0.07 g of Example 11.

MS(ESI, [M+H]⁺) m/z: 816.7.

¹H NMR (500 MHz, DMSO-d₆) δ 11.20 (s, 1H), 11.04 (s, 1H), 7.75 (d, J=2.9 Hz, 1H), 7.65 (s, 1H), 7.54 (d, J=8.6 Hz, 1H), 7.49 (d, J=8.2 Hz, 2H), 7.33 (d, J=2.9 Hz, 1H), 7.12 (d, J=8.2 Hz, 2H), 6.52-6.43 (m, 2H), 4.43 (dd, J=11.4, 5.0 Hz, 1H), 4.37 (d, J=12.2 Hz, 1H), 4.28 (d, J=12.7 Hz, 1H), 3.99 (t, J=7.5 Hz, 2H), 3.67-3.55 (m, 3H), 3.36-3.30 (m, 6H), 3.26 (dd, J=9.2, 7.2 Hz, 2H), 2.98 (dt, J=35.3, 11.6 Hz, 4H), 2.75 (dd, J=11.8, 5.5 Hz, 2H), 2.72 (s, 3H), 2.58 (dt, J=17.2, 4.4 Hz, 1H), 2.45-2.33 (m, 2H), 2.16 (dq, J=13.0, 5.0 Hz, 1H), 1.96 (d, J=11.0 Hz, 2H), 1.85-1.73 (m, 3H), 1.69 (d, J=12.4 Hz, 2H), 1.59-1.45 (m, 3H), 1.37 (q, J=11.1, 9.5 Hz, 2H).

¹³C NMR (126 MHz, DMSO-d₆) δ 173.60, 172.08, 169.70, 165.05, 160.78, 156.42, 154.03, 154.01, 150.97, 140.88, 137.73, 127.40, 122.90, 120.14, 118.61, 114.69, 111.25, 110.24, 89.45, 65.83, 56.17, 55.39, 49.12, 47.19, 45.45, 44.85, 42.69, 39.02, 35.91, 31.54, 31.40, 30.98, 28.22, 24.47, 23.29.

### Example 12: Synthesis of Compound 12

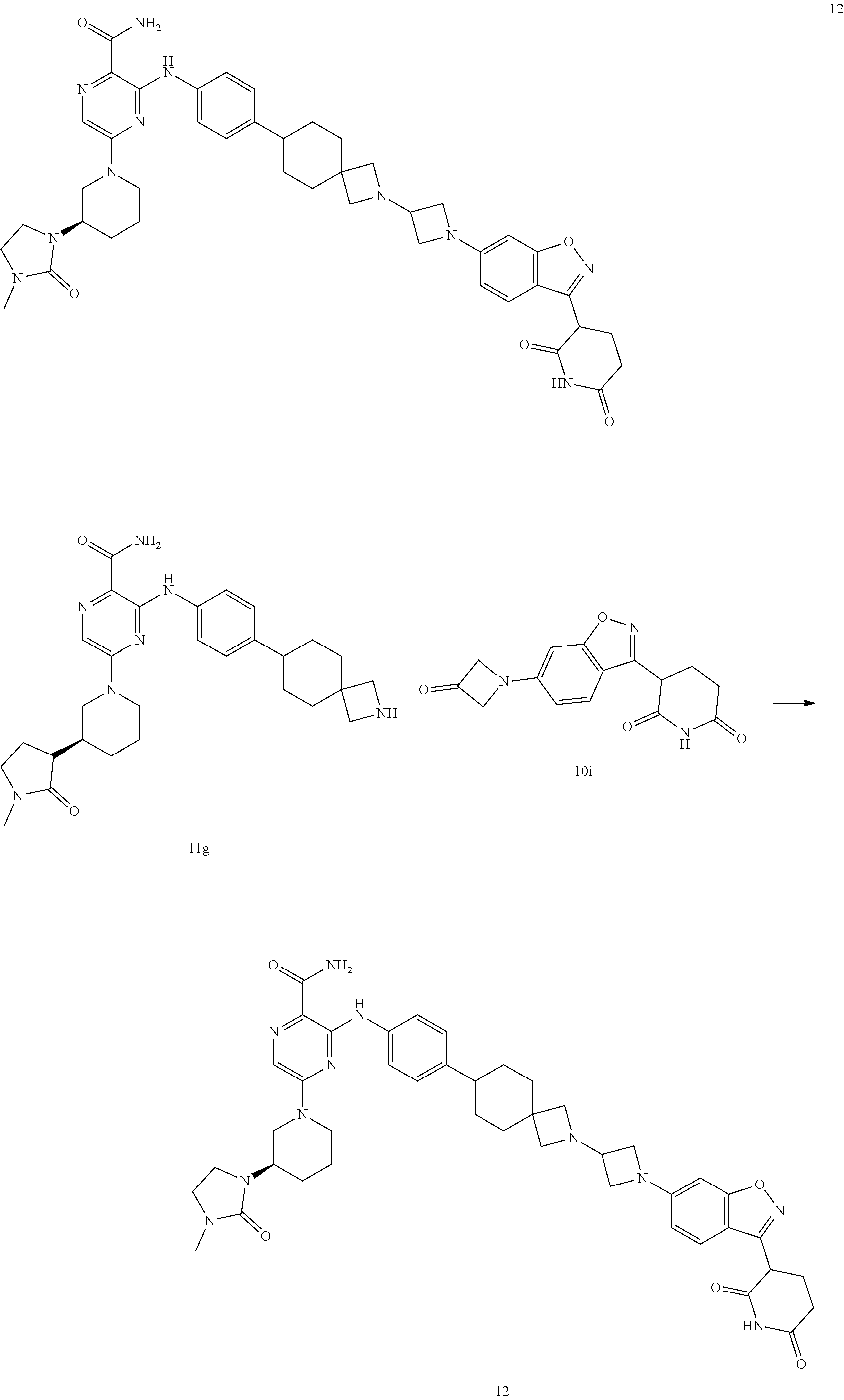 [SMILES: CN1CCN([C@@H]2CCCN(c3cnc(C(N)=O)c(Nc4ccc(C5CCC6(CC5)CN(C5CN(c7ccc8c(C9CCC(=O)NC9=O)noc8c7)C5)C6)cc4)n3)C2)C1=O ; CN1CCC([C@@H]2CCCN(c3cnc(C(N)=O)c(Nc4ccc(C5CCC6(CC5)CNC6)cc4)n3)C2)C1=O ; O=C1CN(c2ccc3c(C4CCC(=O)NC4=O)noc3c2)C1 ; CN1CCN([C@@H]2CCCN(c3cnc(C(N)=O)c(Nc4ccc(C5CCC6(CC5)CN(C5CN(c7ccc8c(C9CCC(=O)NC9=O)noc8c7)C5)C6)cc4)n3)C2)C1=O]

### Step 1: Preparation of Compound 12

Intermediate 11g (200 mg), DCM (30 mL), intermediate 101 (115 mg), and 3 drops of acetic acid were added to a single-necked flask in sequence. The mixture was reacted at room temperature for 1 h and cooled under an ice bath. NaBH₄ (36.5 mg) was added, and the mixture was reacted at room temperature for 20 h. The reaction solution was poured into a mixed solvent of a DCM/MeOH=10/1 solution and water, and the pH was adjusted to 8 with a saturated aqueous sodium bicarbonate solution. The organic phase was separated, and the aqueous phase was extracted several times with DCM/MeOH=10/1. The organic phases were combined, washed with saturated brine, dried over anhydrous sodium sulfate, and filtered. The filtrate was concentrated by evaporation at reduced pressure to remove the solvent, and the crude product was purified by silica gel column chromatography (dichloromethane:methanol=20:1, v/v). The resulting purified product was dissolved in DMSO, purified by (120 g) commercial Cis reversed-phase column, and purified by Biotage medium and low pressure chromatography (1 M aqueous ammonium acetate solution:acetonitrile=1:1, v/v) to give 61 mg of compound 12.

HRMS (ESI) m/z [M+H]⁺:802.41917.

¹H NMR (500 MHz, DMSO-d₆) δ 11.20 (s, 1H), 11.04 (s, 1H), 7.75 (d, J=2.9 Hz, 1H), 7.65 (s, 1H), 7.55 (d, J=8.6 Hz, 1H), 7.48 (d, J=8.3 Hz, 2H), 7.33 (d, J=2.9 Hz, 1H), 7.11 (d, J=8.3 Hz, 2H), 6.52 (d, J=1.8 Hz, 1H), 6.48 (dd, J=8.6, 1.9 Hz, 1H), 4.43 (dd, J=11.4, 5.0 Hz, 1H), 4.36 (d, J=12.5 Hz, 1H), 4.28 (d, J=13.3 Hz, 1H), 3.94 (t, J=7.6 Hz, 2H), 3.77-3.68 (m, 2H), 3.61 (ddt, J=15.4, 11.2, 4.6 Hz, 2H), 3.32-3.20 (m, 4H), 3.08-2.92 (m, 5H), 2.74 (td, J=11.5, 5.7 Hz, 1H), 2.69 (s, 3H), 2.58 (dt, J=17.3, 4.5 Hz, 1H), 2.41 (dddt, J=20.9, 14.7, 9.7, 3.9 Hz, 2H), 2.17 (dq, J=13.3, 5.0 Hz, 1H), 1.97 (d, J=12.3 Hz, 2H), 1.84-1.66 (m, 5H), 1.59-1.47 (m, 3H), 1.42-1.33 (m, 2H), 1.23 (s, 1H).

¹³C NMR (126 MHz, DMSO-d₆) δ 173.62, 172.10, 169.71, 165.08, 160.78, 156.44, 154.04, 153.66, 150.98, 140.92, 137.72, 127.42, 122.93, 120.16, 118.62, 114.69, 111.25, 110.34, 89.52, 61.21, 59.19, 55.12, 53.85, 49.11, 47.18, 45.44, 44.85, 42.71, 39.01, 36.60, 35.11, 31.51, 31.40, 31.01, 28.22, 24.47, 23.29.

### Example 13: Preparation of Compound 13

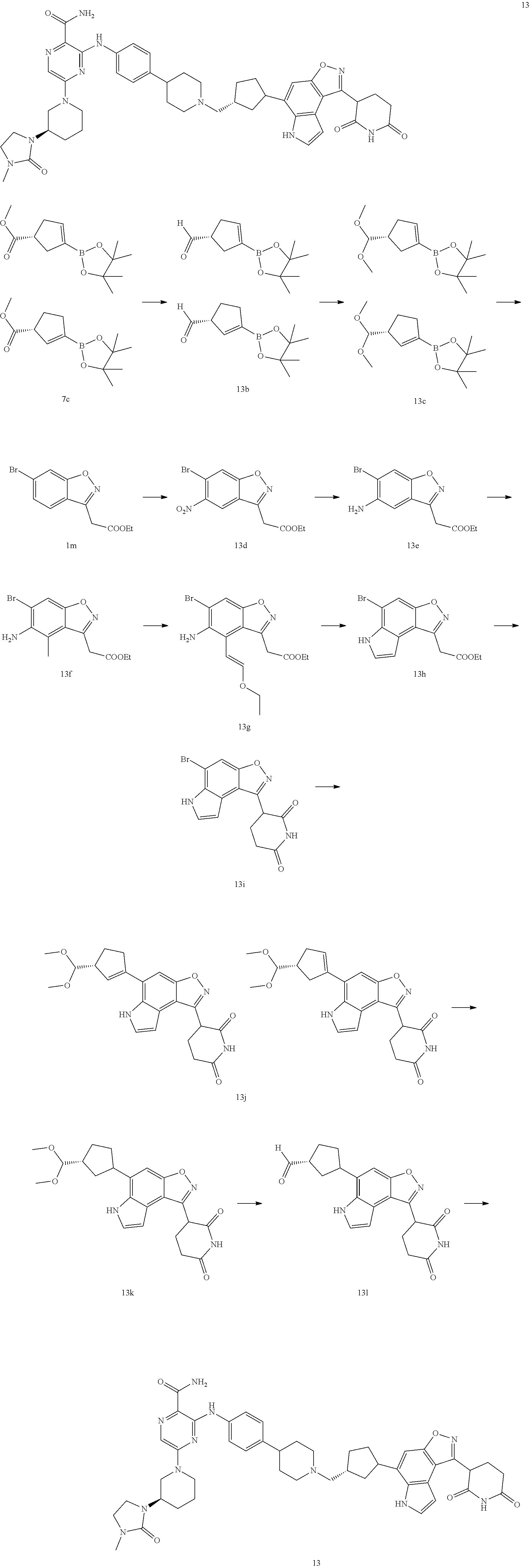

### Step 1: Preparation of Intermediate 13b

DIBAL-H (0.311 g) was slowly added dropwise to a stirred solution of intermediate 7c (0.5 g) in DCM (10 mL) at −78° C. under N₂ atmosphere. After the dropwise addition was carried out for 3 min and completed, the mixture was stirred and reacted at −78° C. for 1 h. 2 mL of methanol was slowly added to the reaction solution at −78° C. to quench the reaction. The reaction solution was placed at room temperature, diluted with 20 mL of petroleum ether, stirred for 5 min, and filtered. The filtrate was concentrated to give mixture intermediate 13b (0.4 g).

### Step 2: Preparation of Intermediate 13c

Intermediate 13b (0.4 g), trimethyl orthoformate (0.261 g), p-toluenesulfonic acid (0.028 g), and methanol (10 mL) were added to a reaction flask in sequence, and the mixture was reacted at room temperature overnight. About 100 mL of a saturated aqueous sodium bicarbonate solution was added to the system, and the mixture was extracted 3 times with 50 mL of ethyl acetate. The organic phases were combined, dried over anhydrous sodium sulfate, filtered, and concentrated. The concentrate was separated and purified by silica gel column chromatography to give mixture intermediate 13c (0.32 g).

¹H NMR (500 MHz, DMSO-d₆) δ 6.33-6.28 (m, 1H), 4.13 (d, J=7.6 Hz, 1H), 3.23 (d, J=1.3 Hz, 6H), 2.50-2.47 (m, 1H), 2.40 (dddt, J=18.3, 15.5, 8.8, 2.4 Hz, 2H), 2.27-2.14 (m, 2H), 1.19 (s, 12H).

### Step 3: Preparation of Intermediate 13d

Intermediate 1m (57 g) and sulfuric acid (200 mL) were added to a reaction flask in sequence, and a mixed solution of nitric acid (25.28 g, 401.25 mmol) and sulfuric acid (20 mL) was slowly added under an ice bath. After the dropwise addition was completed, the mixture was slowly warmed to room temperature and reacted for 1 h. The reaction solution was slowly poured into 2 L of ice water, the mixture was extracted with 500 mL of ethyl acetate, the organic phase was collected, and the aqueous phase was extracted 2 times with 500 m of ethyl acetate. The organic phases were combined, dried over anhydrous sodium sulfate, filtered, and concentrated. The concentrate was separated and purified by silica gel column chromatography to give intermediate 13d (53.3 g).

MS (ESI, [M−H]⁻) m/z: 326.9.

¹H NMR (500 MHz, DMSO-d₆) δ 8.75 (s, 1H), 8.51 (s, 1H), 4.29 (s, 2H), 4.15 (q, J=7.1 Hz, 2H), 1.20 (t, J=7.1 Hz, 3H).

### Step 4: Preparation of Intermediate 13e

Intermediate 13d (40 g), ethanol (400 mL), and stannous chloride dihydrate (115 g) were added to a reaction flask in sequence, and the mixture was stirred at room temperature for 4 h. The reaction solution was concentrated, and 2 L of dichloromethane and 2 L of water were added to the residue. A saturated aqueous sodium bicarbonate solution was slowly added under an ice bath to adjust the pH to 9-10. The mixture was filtered, and the organic phase of the filtrate was separated, dried over anhydrous sodium sulfate, filtered, and concentrated. The concentrate was separated and purified by silica gel column chromatography to give intermediate 13e (35.1 g).

MS(ESI, [M+H]⁺) m/z: 299.0.

¹H NMR (500 MHz, DMSO-d₆) δ 7.93 (s, 1H), 7.04 (s, 1H), 5.35 (s, 2H), 4.14 (q, J=7.1 Hz, 2H), 4.09 (s, 2H), 1.20 (t, J=7.1 Hz, 3H).

### Step 5: Preparation of Intermediate 13f

Silver sulfate (29.46 g), elemental iodine (23.98 g), and acetonitrile (300 mL) were added to a reaction flask in sequence, intermediate 13e (35 g) was then added, and the mixture was reacted at room temperature for 1 h. The reaction solution was filtered, and after the filtrate was concentrated, 500 mL of ethyl acetate and 500 mL of water were added. The organic phase was separated, dried over anhydrous sodium sulfate, filtered, and concentrated. The concentrate was separated and purified by silica gel column chromatography to give intermediate 13f (27.1 g).

MS(ESI, [M+H]⁺) m/z: 423.0.

¹H NMR (500 MHz, DMSO-d₆) δ 8.10 (s, 1H), 5.27 (s, 2H), 4.15 (d, J=6.6 Hz, 4H), 1.22 (t, J=7.1 Hz, 3H).

### Step 6: Preparation of Intermediate 13g

Intermediate 13f (26.8 g), (E)-1-ethoxyvinyl-2-boronic acid pinacol ester (14.99 g), [1,1′-bis(diphenylphosphino)ferrocene]palladium dichloride dichloromethane complex (4.61 g), potassium carbonate (26.14 g), 1,4-dioxane (200 mL), and water (30.00 mL) were added to a reaction flask in sequence, and the mixture was heated to 70° C. and reacted under N₂ atmosphere for 6 h. The reaction solution was cooled to room temperature, and 500 mL of ethyl acetate and 500 mL of water were added. The organic phase was separated, dried over anhydrous sodium sulfate, filtered, and concentrated. The concentrate was separated and purified by silica gel column chromatography to give intermediate 13g (17 g).

MS(ESI, [M+H]⁺) m/z: 369.1.

### Step 7: Preparation of Intermediate 13h

Intermediate 13g (17 g), DCM (200 mL), hydrochloric acid (57.55 mL, 4 mol/L, 230.22 mmol) were added to a reaction flask in sequence, and the mixture was reacted at room temperature for 5 h. 200 mL of DCM and 200 mL of a saturated sodium bicarbonate solution were added to the system. The organic phase was separated, and the aqueous phase was then extracted 2 times with 50 mL of dichloromethane. The organic phases were combined, dried over anhydrous sodium sulfate, filtered and concentrated. The concentrate was separated and purified by silica gel column chromatography to give intermediate 13h (7.1 g).

MS(ESI, [M+H]⁺) m/z: 323.0.

¹H NMR (500 MHz, DMSO-d₆) δ 11.89 (s, 1H), 7.84 (d, J=1.7 Hz, 1H), 7.63 (t, J=2.8 Hz, 1H), 6.77 (dd, J=2.9, 1.6 Hz, 1H), 4.29 (s, 2H), 4.13 (q, J=7.1 Hz, 2H), 1.19-1.16 (m, 3H).

### Step 8: Preparation of Intermediate 13i

Intermediate 13h (3 g), acrylamide (0.792 g), and anhydrous tetrahydrofuran (50 mL) were added to a reaction flask in sequence. Potassium tert-butoxide (1.56 g) was slowly added at 0° C., and the mixture was reacted for 2 h. 200 mL of a saturated aqueous ammonium chloride solution was added to the reaction solution to quench the reaction, and the mixture was extracted with 200 mL of DCM. The organic phase was separated, and the aqueous phase was extracted 2 times with 50 mL of DCM. The organic phases were combined, dried over anhydrous sodium sulfate, filtered and concentrated. The concentrate was separated and purified by silica gel column chromatography to give intermediate 13i (1.34 g).

MS(ESI, [M−H]⁻) m/z: 345.9.

¹H NMR (500 MHz, DMSO-d₆) δ 11.91 (s, 1H), 11.19 (s, 1H), 7.86 (s, 1H), 7.63 (t, J=2.9 Hz, 1H), 6.71 (dd, J=3.0, 1.8 Hz, 1H), 4.72 (dd, J=12.0, 5.1 Hz, 1H), 2.85 (ddd, J=17.5, 12.2, 5.4 Hz, 1H), 2.64 (dt, J=17.3, 4.1 Hz, 1H), 2.48-2.36 (m, 1H), 2.24 (dtd, J=13.7, 5.2, 3.6 Hz, 1H).

### Step 9: Preparation of Intermediate 13j

Intermediate 13i (0.2 g), intermediate 13c (0.185 g), [1,1′-bis(diphenylphosphino)ferrocene]palladium dichloride dichloromethane complex (0.042 g), potassium carbonate (0.183 g), 1,4-dioxane (10 mL), and water (2.00 mL) were added to a reaction flask in sequence, and the mixture was heated to 120° C. and reacted under N₂ atmosphere for 3 h. The reaction solution was cooled to room temperature, and 500 mL of ethyl acetate and 500 mL of water were added. The organic phase was separated, dried over anhydrous sodium sulfate, filtered, and concentrated. The concentrate was separated and purified by silica gel column chromatography to give mixture intermediate 13j (0.205 g).

MS (ESI, [M−H]⁻) m/z: 408.2.

### Step 10: Preparation of Intermediate 13k

Intermediate 13j (0.39 g), palladium on carbon catalyst (0.039 g), and MeOH (20 mL) were added to a reaction flask in sequence, and the mixture was reacted at room temperature overnight under H₂ atmosphere. The reaction solution was filtered, and the filtrate was concentrated to give intermediate 13k (0.36 g).

MS (ESI, [M−H]⁻) m/z: 410.2.

¹H NMR (500 MHz, DMSO-d₆) δ 11.61 (s, 1H), 11.17 (s, 1H), 7.52 (t, J=2.8 Hz, 1H), 7.33 (d, J=9.7 Hz, 1H), 6.57-6.52 (m, 1H), 4.64 (dd, J=11.8, 5.1 Hz, 1H), 4.29 (d, J=7.6 Hz, 1H), 3.61 (ddd, J=24.3, 10.6, 5.4 Hz, 1H), 3.28 (dd, J=7.6, 5.4 Hz, 6H), 2.84 (ddd, J=17.2, 12.0, 5.4 Hz, 1H), 2.62 (dt, J=17.3, 4.2 Hz, 1H), 2.43 (tt, J=12.1, 7.0 Hz, 2H), 2.22 (dq, J=13.8, 7.7, 5.9 Hz, 2H), 2.16-2.09 (m, 1H), 1.87-1.67 (m, 3H), 1.60-1.51 (m, 1H).

### Step 11: Preparation of Intermediate 13l

Intermediate 13k (0.1 g), acetone (5 mL), and p-toluenesulfonic acid (0.021 g) were added to a reaction flask in sequence, and the mixture was reacted at room temperature for 2 h. 50 mL of a saturated aqueous sodium bicarbonate solution and 50 mL of DCM were added to the system. The organic phase was separated, dried over anhydrous sodium sulfate, filtered, and concentrated to give intermediate 13l (0.085 g).

MS (ESI, [M−H]⁻) m/z: 364.2.

### Step 12: Preparation of Compound 13

Intermediate 13l (0.08 g), intermediate 1l (0.104 g), and dichloroethane (5 mL) were added to a reaction flask, 1 drop of acetic acid was added, and sodium cyanoborohydride (0.026 g) was then added. The mixture was reacted at room temperature for 2 h. After the reaction was completed, dichloromethane (50 mL) and water (50 mL) were added to the system. The organic phase was separated, and the aqueous phase was extracted 2 times with dichloromethane (50 mL). The organic phases were combined, dried over anhydrous sodium sulfate, filtered and concentrated. The concentrate was separated and purified by silica gel column chromatography to give compound 13 (0.042 g).

MS (ESI, [M+H]⁺) m/z: 828.4.

¹H NMR (500 MHz, DMSO-d₆) δ 11.73 (d, J=27.7 Hz, 1H), 11.28 (s, 1H), 11.17 (s, 1H), 7.78 (s, 1H), 7.67 (s, 1H), 7.56 (d, J=7.5 Hz, 3H), 7.40 (s, 1H), 7.35 (s, 1H), 7.18 (d, J=8.0 Hz, 2H), 6.56 (s, 1H), 4.66 (dd, J=11.7, 5.2 Hz, 1H), 4.39-4.27 (m, 2H), 3.71 (s, 1H), 3.62 (s, 3H), 3.26 (s, 3H), 3.07 (d, J=11.6 Hz, 2H), 2.97 (t, J=12.5 Hz, 1H), 2.84 (d, J=14.6 Hz, 1H), 2.71 (s, 3H), 2.63 (d, J=16.9 Hz, 2H), 2.43 (s, 1H), 2.23 (s, 2H), 2.00 (s, 3H), 1.82 (d, J=12.9 Hz, 4H), 1.62 (d, J=45.5 Hz, 4H).

### Example 14: Synthesis of Compound 14

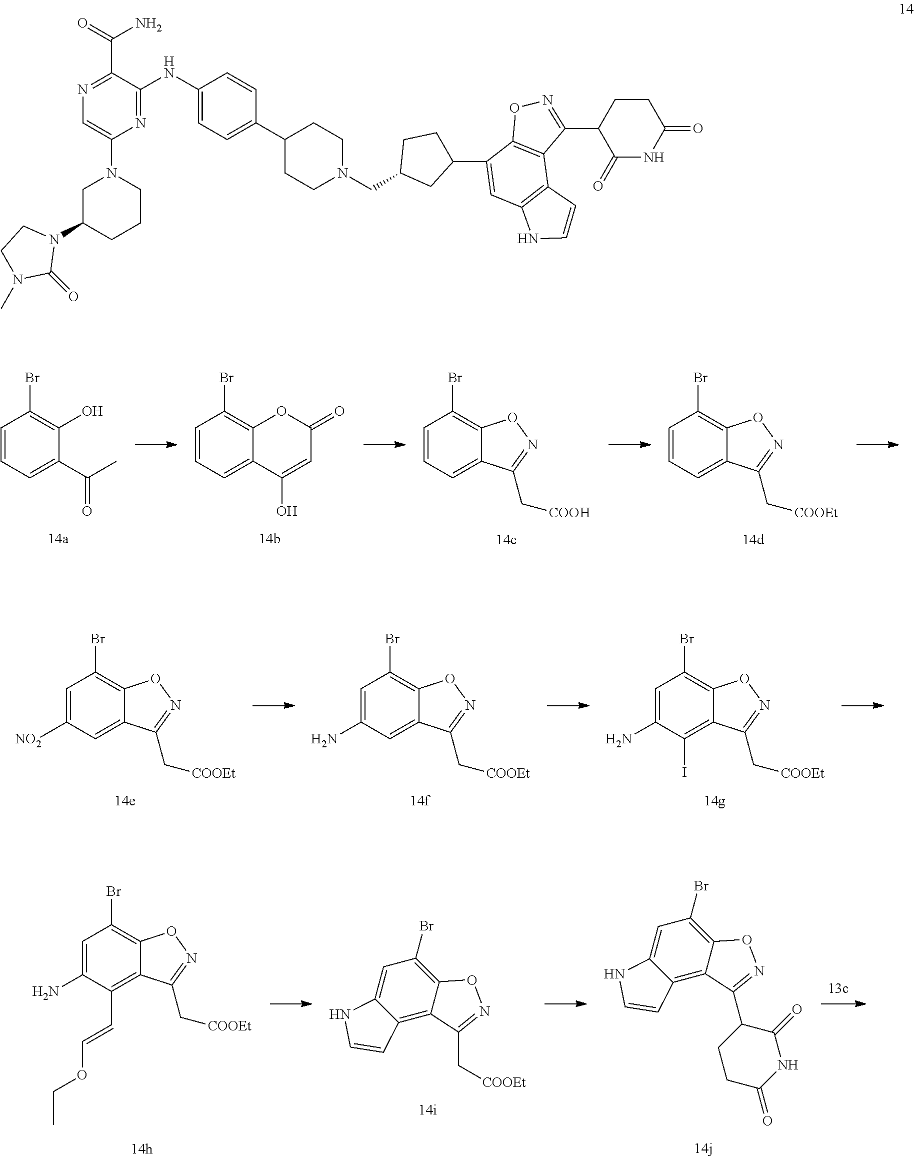

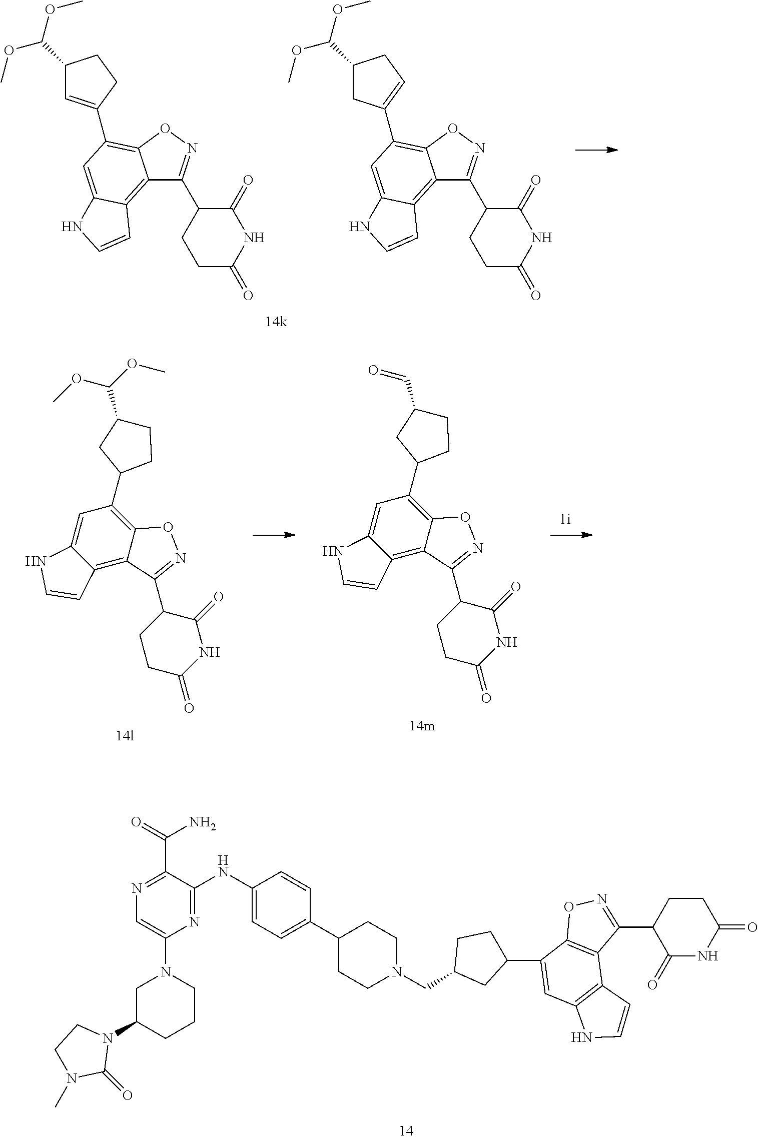 [SMILES: COC(OC)[C@H]1C=C(c2cc3[nH]ccc3c3c(C4CCC(=O)NC4=O)noc23)CC1 ; COC(OC)[C@@H]1CC=C(c2cc3[nH]ccc3c3c(C4CCC(=O)NC4=O)noc23)C1 ; COC(OC)[C@@H]1CCC(c2cc3[nH]ccc3c3c(C4CCC(=O)NC4=O)noc23)C1 ; O=C[C@@H]1CCC(c2cc3[nH]ccc3c3c(C4CCC(=O)NC4=O)noc23)C1 ; CN1CCN([C@@H]2CCCN(c3cnc(C(N)=O)c(Nc4ccc(C5CCN(C[C@@H]6CCC(c7cc8[nH]ccc8c8c(C9CCC(=O)NC9=O)noc78)C6)CC5)cc4)n3)C2)C1=O]

### Step 1: Preparation of Intermediate 14b

14a (50 g), diethyl carbonate (137 g), and toluene (200 mL) were added to a single-necked flask in sequence. The reaction solution was cooled to 0° C. and sodium hydride (46.5 g) was added in portions. The mixture was first heated to 80° C. and reacted for about 10 min. The mixture was heated to 120° C. and reacted for 5 h. The reaction solution was cooled to room temperature and slowly poured into 2 L of stirred ice water. The mixture was extracted with 1 L of ethyl acetate, and the organic phase was discarded. The aqueous phase was adjusted to pH=3 with 3 N hydrochloric acid, the mixture was extracted 3 times with 500 mL of ethyl acetate, and the organic phases were combined. The organic phase was dried over anhydrous sodium sulfate, filtered and concentrated by evaporation at reduced pressure to remove the solvent, thus giving intermediate 14b (52 g).

MS(ESI, [M−H]⁺) m/z: 239.0.

¹H NMR (500 MHz, DMSO-d₆) δ 12.77 (s, 1H), 7.94 (dd, J=7.8, 1.5 Hz, 1H), 7.83 (dd, J=7.9, 1.5 Hz, 1H), 7.29 (t, J=7.9 Hz, 1H), 5.64 (s, 1H).

### Step 2: Preparation of Intermediate 14c

Intermediate 14b (52 g), methanol (300 mL), hydroxylamine hydrochloride (52.5 g), and sodium ethoxide (61.9 g) were added to a single-necked flask in sequence, and the mixture was heated to 80° C. and reacted overnight. The reaction solution was cooled to room temperature, 3 N hydrochloric acid was added to adjust the pH to 5, the mixture was concentrated by evaporation at reduced pressure to remove the solvent, 2 L of water was added, the reaction flask was placed under an ice-water bath to cool, and meanwhile the pH was adjusted to 3 with 3 N hydrochloric acid. The mixture was stirred for 30 min and filtered. The filter cake was collected and dried to give intermediate 14c (46 g).

MS(ESI, [M−H]⁺) m/z: 254.0.

### Step 3: Preparation of Intermediate 14d

Intermediate 14c (46 g), ethanol (400 mL), and sulfuric acid (106 g, 57.5 mL, 1078 mmol) were added to a single-necked flask in sequence, and the mixture was heated to 90° C. and reacted for 2 h. The reaction solution was cooled to room temperature and concentrated by evaporation at reduced pressure to remove the solvent, and 1 L of ethyl acetate and 1 L of water were added to the residue for dilution. A saturated aqueous sodium bicarbonate solution was added to adjust the pH to 7, and the organic phase was separated. The aqueous phase was extracted 2 times with 500 mL of ethyl acetate, the organic phases were combined, dried over anhydrous sodium sulfate, filtered and concentrated by evaporation at reduced pressure to remove the solvent, thus giving intermediate 14d (51 g).

MS(ESI, [M+H]⁺) m/z: 283.0.

¹H NMR (500 MHz, DMSO-d₆) δ 7.93 (dd, J=7.6, 0.9 Hz, 1H), 7.89 (dd, J=7.9, 0.9 Hz, 1H), 7.38 (t, J=7.8 Hz, 1H), 4.26 (s, 2H), 4.15 (q, J=7.1 Hz, 2H), 1.20 (t, J=7.1 Hz, 3H).

### Step 4: Preparation of Intermediate 14e

Intermediate 14d (51 g), sulfuric acid (176 g, 96 mL, 1795 mmol), and potassium nitrate (27.2 g) were added to a single-necked flask in sequence under an ice bath. After the feeding was completed, the mixture was reacted at room temperature for 1 h. The reaction solution was slowly poured into 2 L of ice water, the mixture was extracted with 500 mL of ethyl acetate, the organic phase was collected, and the aqueous phase was extracted 2 times with 500 mL of ethyl acetate. The organic phases were combined, dried over anhydrous sodium sulfate, filtered, and concentrated by evaporation at reduced pressure to remove the solvent. The crude product was separated by silica gel column chromatography (petroleum ether/ethyl acetate=20:1, v/v) to give the target intermediate 14e (40 g).

¹H NMR (500 MHz, DMSO-d₆) δ 8.98 (d, J=2.1 Hz, 1H), 8.75 (d, J=2.1 Hz, 1H), 4.38 (s, 2H), 4.17 (q, J=7.1 Hz, 2H), 1.22 (t, J=7.1 Hz, 3H).

### Step 5: Preparation of Intermediate 14f

Intermediate 14e (40 g), ethanol (400 mL), and stannous chloride dihydrate (115 g) were added to a single-necked flask in sequence, and the mixture was stirred at room temperature for 4 h. The mixture was concentrated by evaporation at reduced pressure to remove the solvent, and 2 L of dichloromethane and 2 L of water were added to the residue. A saturated aqueous sodium bicarbonate solution was slowly added under an ice bath to adjust the pH to weak alkalinity. The mixture was filtered, the filter cake was washed 2 times with 500 mL of dichloromethane, and the filtrate was collected. After liquid separation was performed, the organic phase was collected, and the aqueous phase was then extracted 2 times with 500 mL of dichloromethane. The organic phases were combined, dried over anhydrous sodium sulfate, filtered, and concentrated by evaporation at reduced pressure to remove the solvent. The crude product was separated by silica gel column chromatography (petroleum ether/ethyl acetate=2:1, v/v) to give the target intermediate 14 (23 g).

MS(ESI, [M+H]⁺) m/z: 299.0.

### Step 6: Preparation of Intermediate 14g

Intermediate 14 (6 g), DMF (50 mL), and NIS (4.51 g) were added to a single-necked flask in sequence, and the mixture was reacted at room temperature for 1 h. 100 mL of ethyl acetate and 200 mL of water were added to the system. The organic phase was separated. The aqueous phase was then extracted 2 times with 50 mL of ethyl acetate, and the organic phases were combined, dried over anhydrous sodium sulfate, filtered and concentrated by evaporation at reduced pressure to remove the solvent. The crude product was separated by silica gel column chromatography (petroleum ether/ethyl acetate=4:1, v/v) to give the target intermediate 14g (8 g).

MS(ESI, [M−H]⁺) m/z: 423.0.

¹H NMR (500 MHz, DMSO-d₆) δ 7.36 (s, 1H), 5.54 (s, 2H), 4.21-4.10 (m, 4H), 1.22 (t, J=7.1 Hz, 3H).

### Step 7: Preparation of Intermediate 14h

Intermediate 14g (4 g), (E)-1-ethoxyvinyl-2-boronic acid pinacol ester (2.237 g), [1,1′-bis(diphenylphosphino)ferrocene]palladium dichloride dichloromethane complex (1.377 g), potassium carbonate (3.90 g), 1,4-dioxane (40 mL), and water (10.00 mL) were added to a single-necked flask in sequence, and the mixture was heated to 80° C. and reacted under N₂ atmosphere overnight. The reaction solution was cooled to room temperature, and ethyl acetate (100 mL) and water (100 mL) were added to the system. The organic phase was separated and then extracted 3 times with 50 mL of ethyl acetate, and the organic phases were combined, dried over anhydrous sodium sulfate, filtered and concentrated by evaporation at reduced pressure to remove the solvent. The crude product was separated by silica gel column chromatography (petroleum ether/ethyl acetate=4:1, v/v) to give the target intermediate 14h (2.4 g).

MS(ESI, [M+H]⁺) m/z: 369.1.

### Step 8: Preparation of Intermediate 141

Intermediate 14h (1 g), dichloromethane (10 mL), and trifluoroacetic acid (1.544 g, 1.043 mL) were added to a single-necked flask in sequence, and the mixture was reacted at room temperature overnight. Dichloromethane (100 mL) and water (100 mL) were added to the system. The organic phase was separated. The aqueous phase was then extracted 2 times with dichloromethane (50 mL), and the organic phases were combined, dried over anhydrous sodium sulfate, filtered and concentrated by evaporation at reduced pressure to remove the solvent. The crude product was separated by silica gel column chromatography (petroleum ether/ethyl acetate=4:1, v/v) to give the target intermediate 141 (0.5 g).

MS(ESI, [M+H]⁺) m/z: 323.0.

¹H NMR (500 MHz, DMSO-d₆) δ 11.78 (s, 1H), 7.96 (s, 1H), 7.61 (t, J=2.9 Hz, 1H), 6.66 (t, J=2.3 Hz, 1H), 4.31 (s, 2H), 4.14 (q, J=7.1 Hz, 2H), 1.16 (t, J=7.1 Hz, 3H).

### Step 9: Preparation of Intermediate 14j

Intermediate 141 (300 mg), acrylamide (66 mg), and anhydrous tetrahydrofuran (5 mL) were added to a three-necked flask in sequence. Potassium tert-butoxide (208 mg) was slowly added at 0° C., and the mixture was reacted at 0° C. for 2 h. A saturated aqueous ammonium chloride solution was added dropwise to the reaction solution to quench the reaction, and the mixture was extracted with 50 mL of ethyl acetate. The organic phase was separated. The aqueous phase was extracted 3 times with ethyl acetate (50 mL), the organic phases were combined, dried over anhydrous sodium sulfate, filtered and concentrated by evaporation at reduced pressure to remove the solvent. The crude product was separated by silica gel column chromatography (petroleum ether/ethyl acetate=3:2, v/v) to give the target intermediate 14j (0.18 g).

MS(ESI, [M−H]⁺) m/z: 345.9.

¹H NMR (500 MHz, DMSO-d₆) δ 11.80 (s, 1H), 11.20 (s, 1H), 7.97 (d, J=0.8 Hz, 1H), 7.61 (t, J=2.8 Hz, 1H), 6.60 (t, J=2.3 Hz, 1H), 4.73 (dd, J=12.2, 5.1 Hz, 1H), 2.86 (ddd, J=17.5, 12.3, 5.4 Hz, 1H), 2.65 (dt, J=17.3, 4.0 Hz, 1H), 2.45 (qd, J=12.5, 4.4 Hz, 1H), 2.25 (dtd, J=13.6, 5.2, 3.4 Hz, 1H).

### Step 10: Preparation of Intermediate 14k

Intermediate 14j (480 mg), intermediate 13c (370 mg), [1,1′-bis(diphenylphosphino)ferrocene]palladium dichloride dichloromethane complex (202 mg), potassium carbonate (572 mg), 1,4-dioxane (5 mL), and water (0.5 mL) were added to a single-necked flask in sequence, and the mixture was heated to 85° C. and reacted under N₂ atmosphere for 2 h. The reaction solution was cooled to room temperature, and ethyl acetate (50 mL) and water (100 mL) were added to the system for extraction. The organic phase was separated, then extracted 3 times with ethyl acetate (50 mL), dried over anhydrous sodium sulfate, filtered, and concentrated by evaporation at reduced pressure to remove the solvent. The crude product was separated by silica gel column chromatography (dichloromethane/methanol=60:1, v/v) to give mixture intermediate 14k (0.35 g).

MS(ESI, [M−H]⁺) m/z: 408.19.

¹H NMR (500 MHz, DMSO-d₆) δ 11.63 (d, J=30.9 Hz, 1H), 11.18 (s, 1H), 7.57 (dt, J=9.7, 3.5 Hz, 2H), 6.64 (d, J=6.3 Hz, 1H), 6.62-6.48 (m, 1H), 4.71 (dt, J=11.4, 5.0 Hz, 1H), 4.38-4.21 (m, 1H), 3.33-3.25 (m, 6H), 3.00-2.79 (m, 2H), 2.79-2.58 (m, 3H), 2.44 (tt, J=12.2, 6.3 Hz, 1H), 2.25 (dp, J=13.1, 5.2 Hz, 1H), 2.16-1.96 (m, 1H), 1.88-1.72 (m, 1H).

### Step 11: Preparation of Intermediate 141

Intermediate 14k (50 mg), methanol (3 mL), and Pd/C (10 mg) were added to a single-necked flask in sequence, and the mixture was purged 3 times with hydrogen and reacted at room temperature for 3 h. The reaction solution was filtered to remove palladium on carbon, the filter cake was washed with 5 mL each of dichloromethane, methanol, and ethyl acetate, and the filtrate was distilled under reduced pressure to remove the solvent, thus giving intermediate 141 (40 mg).

MS(ESI, [M−H]⁺) m/z: 410.21.

¹H NMR (500 MHz, DMSO-d₆) δ 11.52 (d, J=2.3 Hz, 1H), 11.18 (s, 1H), 7.54 (s, 1H), 7.47 (t, J=2.8 Hz, 1H), 6.49 (q, J=2.3 Hz, 1H), 4.68 (ddd, J=11.9, 9.1, 5.2 Hz, 1H), 4.28 (d, J=7.5 Hz, 1H), 3.55-3.41 (m, 1H), 3.29 (dd, J=7.2, 2.3 Hz, 6H), 2.86 (ddd, J=17.3, 12.1, 5.4 Hz, 1H), 2.64 (dt, J=17.3, 4.2 Hz, 1H), 2.49-2.38 (m, 2H), 2.29-2.09 (m, 3H), 1.89-1.75 (m, 2H), 1.74-1.61 (m, 2H).

### Step 12: Preparation of Intermediate 14m

Intermediate 141 (100 mg), bis(acetonitrile) palladium dichloride (31.5 mg), and acetone (10 mL) were added to a single-necked flask in sequence, and the mixture was reacted at room temperature for 1 h. Ethyl acetate (30 mL) and water (50 mL) were added to the system for extraction. The organic phase was separated. The aqueous phase was then extracted 3 times with ethyl acetate (30 mL), and the organic phases were combined, dried over anhydrous sodium sulfate, filtered and concentrated by evaporation at reduced pressure to remove the solvent, thus giving intermediate 14m (70 mg).

MS(ESI, [M−H]⁺) m/z: 364.11

### Step 13: Preparation of Compound 14

Intermediate 1l (90 mg), intermediate 14m (68.7 mg), and 1,2-dichloroethane (2 mL) were added to a single-necked flask in sequence, 1 drop of acetic acid was added dropwise, and the mixture was stirred at room temperature for 20 min. Sodium cyanoborohydride (35.5 mg) was added, and the mixture was reacted at room temperature. Dichloromethane (20 mL) and water (50 mL) were added to the system for extraction. The organic phase was separated, and the aqueous phase was extracted 3 times with DCM/MeOH=10:1 (30 mL). The organic phases were combined, dried over anhydrous sodium sulfate, filtered, concentrated by evaporation at reduced pressure to remove the solvent, and purified by silica gel column chromatography to give compound 14 (20 mg).

MS(ESI, [M+H]⁺) m/z: 828.52.

¹H NMR (500 MHz, DMSO-d₆) δ 11.62 (d, J=27.7 Hz, 1H), 11.28 (s, 1H), 11.19 (s, 1H), 7.89 (s, 1H), 7.68 (s, 1H), 7.56 (d, J=7.5 Hz, 3H), 7.49 (s, 1H), 7.35 (s, 1H), 7.18 (d, J=8.0 Hz, 2H), 6.51 (s, 1H), 4.68 (dd, J=11.7, 5.2 Hz, 1H), 4.35-4.25 (m, 2H), 3.76 (s, 1H), 3.61 (s, 3H), 3.31 (s, 3H), 3.13 (d, J=11.6 Hz, 2H), 2.91 (t, J=12.5 Hz, 1H), 2.79 (d, J=14.6 Hz, 1H), 2.79 (s, 3H), 2.59 (d, J=16.9 Hz, 2H), 2.49 (s, 1H), 2.26 (s, 2H), 2.05 (s, 3H), 1.83 (d, J=12.9 Hz, 4H), 1.66 (d, J=45.5 Hz, 4H).

### Example 15: Preparation of Compound 15

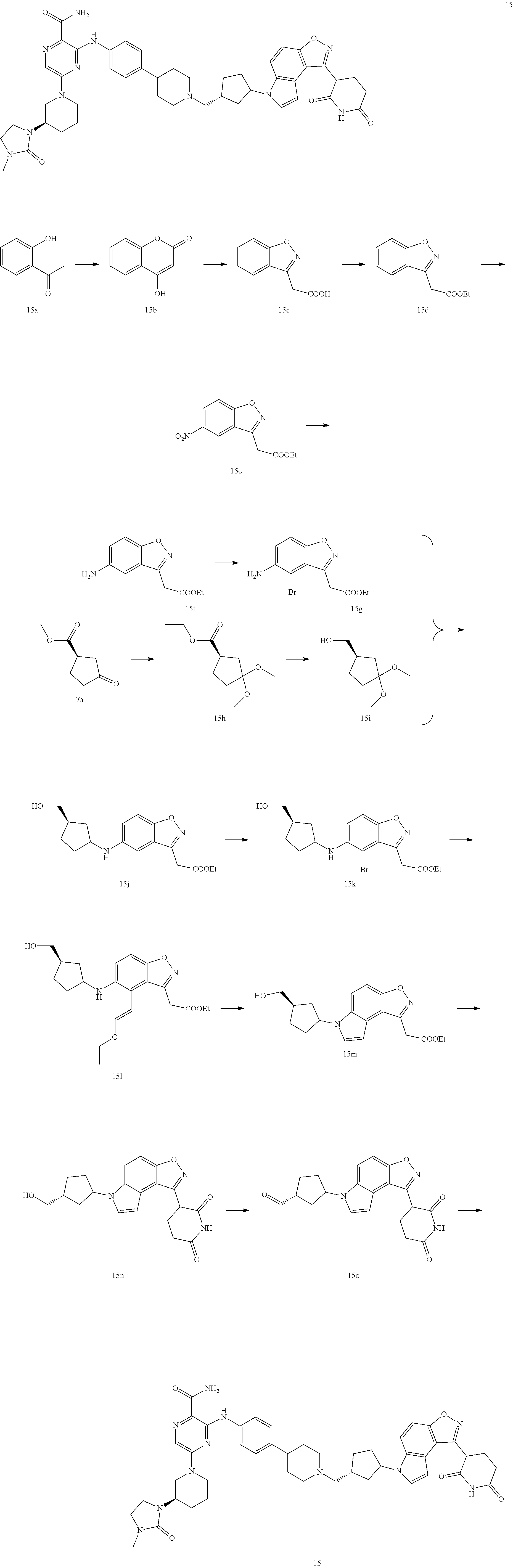

### Step 1: Preparation of Intermediate 15b

15a (50 g), diethyl carbonate (216.9 g), and toluene (500 mL) were added to a reaction flask in sequence. The reaction solution was cooled to 0° C. and sodium hydride (44.06 g) was added in portions. The mixture was first heated to 70° C. and reacted for about 10 min. The mixture was heated to 120° C. and reacted for 5 h. The reaction solution was cooled to room temperature and slowly poured into 2 L of stirred ice water. The mixture was extracted with 1 L of ethyl acetate, and the organic phase was discarded. The aqueous phase was adjusted to pH=3 with 3 N hydrochloric acid, the mixture was extracted 3 times with 500 mL of ethyl acetate, and the organic phases were combined. The organic phase was dried over anhydrous sodium sulfate, filtered and concentrated to give intermediate 15b (55 g).

¹H NMR (500 MHz, DMSO-d₆) δ 12.52 (s, 1H), 7.83 (dd, J=7.8, 1.7 Hz, 1H), 7.65 (ddd, J=8.6, 7.2, 1.7 Hz, 1H), 7.43-7.32 (m, 2H), 5.61 (s, 1H).

### Step 2: Preparation of Intermediate 15c

Intermediate 15b (55 g), methanol (500 mL), hydroxylamine hydrochloride (63.5 g), and sodium ethoxide (80.8 g) were added to a reaction flask in sequence, and the mixture was heated to 80° C. and reacted overnight. The reaction solution was cooled to room temperature, 3 N hydrochloric acid was added to adjust the pH to 5, the mixture was concentrated, 2 L of water was added, the reaction flask was placed under an ice-water bath to cool, and meanwhile the pH was adjusted to 3 with 3 N hydrochloric acid. The mixture was stirred for 30 min and filtered. The filter cake was collected and dried to give intermediate 15c (54.5 g).

¹H NMR (500 MHz, DMSO-d₆) δ 12.90 (s, 1H), 7.86 (dt, J=7.9, 1.0 Hz, 1H), 7.74 (d, J=8.4 Hz, 1H), 7.66 (ddd, J=8.3, 7.0, 1.2 Hz, 1H), 7.40 (td, J=7.4, 7.0, 0.9 Hz, 1H), 4.11 (s, 2H).

### Step 3: Preparation of Intermediate 15d

Intermediate 15c (54 g), ethanol (400 mL), and sulfuric acid (106 g) were added to a reaction flask in sequence, and the mixture was heated to 90° C. and reacted for 2 h. The reaction solution was cooled to room temperature and concentrated by evaporation at reduced pressure to remove the solvent, and 1 L of ethyl acetate and 1 L of water were added to the residue for dilution. A saturated aqueous sodium bicarbonate solution was added to adjust the pH to 7, and the organic phase was separated. The aqueous phase was extracted 2 times with 500 mL of ethyl acetate, the organic phases were combined, dried over anhydrous sodium sulfate, filtered and concentrated to give intermediate 15d (62 g).

MS(ESI, [M+H]⁺) m/z: 206.1.

### Step 4: Preparation of Intermediate 15e

Intermediate 15d (30 g) and sulfuric acid (200 mL) were added to a reaction flask in sequence, and a mixed solution of nitric acid (11.05 g) and sulfuric acid (4 mL) was slowly added under an ice bath. After the dropwise addition was completed, the mixture was slowly warmed to room temperature and reacted for 1 h. The reaction solution was slowly poured into 2 L of ice water, the mixture was extracted with 300 mL of ethyl acetate, the organic phase was collected, and the aqueous phase was extracted 2 times with 100 m of ethyl acetate. The organic phases were combined, dried over anhydrous sodium sulfate, filtered, and concentrated. The concentrate was separated and purified by silica gel column chromatography to give intermediate 15e (29.3 g).

MS(ESI, [M+H]⁺) m/z: 251.1.

### Step 5: Preparation of Intermediate 15f

Intermediate 15e (29 g), ethanol (300 mL), and stannous chloride dihydrate (130.76 g) were added to a reaction flask in sequence, and the mixture was stirred at room temperature for 4 h. The reaction solution was concentrated, and 1 L of dichloromethane and 0.5 L of water were added to the residue. A saturated aqueous sodium bicarbonate solution was slowly added under an ice bath to adjust the pH to 9-10. The mixture was filtered, and the organic phase of the filtrate was separated, dried over anhydrous sodium sulfate, filtered, and concentrated. The concentrate was separated and purified by silica gel column chromatography to give intermediate 1f (24.3 g).

MS(ESI, [M−H]⁻) m/z: 219.1.

### Step 6: Preparation of Intermediate 15g

Intermediate 1f (24 g) and DCM (200 mL) were added to a reaction flask in sequence, NBS (21.34 g) was added at 0° C., and the mixture was reacted at room temperature for 1 h. 500 mL of DCM and 500 mL of water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 15g (22.4 g).

MS(ESI, [M+H]⁺) m/z: 299.2.

¹H NMR (500 MHz, DMSO-d₆) δ 7.52 (d, J=8.9 Hz, 1H), 7.17 (d, J=8.9 Hz, 1H), 4.87 (s, 2H), 4.19-4.11 (m, 4H), 1.20 (t, J=7.1 Hz, 3H).

### Step 7: Preparation of Intermediate 15h

Intermediate 7a (5 g), trimethyl orthoformate (11.2 g), p-toluenesulfonic acid (0.606 g), and ethanol (50 mL) were added to a reaction flask in sequence, and the mixture was reacted at room temperature overnight. About 100 mL of a saturated aqueous sodium bicarbonate solution was added to the system, and the mixture was extracted 3 times with 100 mL of ethyl acetate. The organic phases were combined, dried over anhydrous sodium sulfate, filtered, and concentrated. The concentrate was separated and purified by silica gel column chromatography to give intermediate 15h (4.5 g).

¹H NMR (500 MHz, Chloroform-d) δ 4.14 (q, J=7.1 Hz, 2H), 3.21 (d, J=10.6 Hz, 6H), 2.92-2.81 (m, 1H), 2.14-2.03 (m, 2H), 2.01-1.91 (m, 2H), 1.91-1.78 (m, 2H), 1.25 (t, J=7.1 Hz, 3H).

### Step 8: Preparation of Intermediate 151

Intermediate 15h (4.5 g) and THF (50 mL) were added to a reaction flask, lithium aluminum hydride (0.998 g) was slowly added under an ice bath, and then the mixture was slowly warmed to room temperature and reacted for 1 h. After the reaction was completed, a small amount of ice water was added to the reaction solution under an ice bath to quench the reaction, and 200 mL of DCM and 200 mL of water were then added. The mixture was filtered, and the organic phase of the filtrate was separated, washed with 200 mL of a saturated sodium chloride solution, dried over anhydrous sodium sulfate, filtered, and concentrated to give intermediate 151 (3.9 g).

¹H NMR (500 MHz, Chloroform-d) δ 3.55 (dhept, J=15.8, 5.4, 4.9 Hz, 2H), 3.21 (d, J=3.0 Hz, 6H), 2.28 (dddd, J=15.1, 8.8, 7.6, 3.9 Hz, 1H), 2.05-1.96 (m, 1H), 1.90 (dddd, J=10.2, 8.8, 3.7, 1.5 Hz, 2H), 1.85-1.81 (m, 1H), 1.80-1.73 (m, 1H), 1.58 (ddd, J=13.4, 7.3, 1.3 Hz, 1H), 1.51-1.40 (m, 1H).

### Step 9: Preparation of Intermediate 15j

Intermediate 15g (2.2 g), intermediate 151 (1.3 g), triethylsilane (1.71 g), and acetonitrile (20 mL) were added to a reaction flask, elemental iodine (1.87 g) was then added, and the mixture was warmed to 90° C. and reacted overnight. The reaction solution was cooled to room temperature and concentrated, and 200 mL of DCM and 200 mL of water were added. The organic phase was separated, washed with 200 mL of a saturated sodium chloride solution, dried over anhydrous sodium sulfate, filtered, and concentrated. The concentrate was separated and purified by silica gel column chromatography to give intermediate 15j (1.9 g).

MS (ESI, [M+H]⁺) m/z: 319.2.

### Step 10: Preparation of Intermediate 15k

Intermediate 15j (1.9 g) and DCM (30 mL) were added to a reaction flask in sequence, NBS (1.06 g) was then added, and the mixture was reacted at room temperature for 1 h. 100 mL of DCM and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 15k (0.75 g).

MS(ESI, [M+H]⁺) m/z: 397.2.

¹H NMR (500 MHz, DMSO-d₆) δ 7.63 (dd, J=9.0, 7.4 Hz, 1H), 7.18 (dd, J=9.2, 3.4 Hz, 1H), 4.96 (d, J=7.6 Hz, 1H), 4.70-4.63 (m, 1H), 4.19-4.12 (m, 4H), 3.92 (hept, J=6.3 Hz, 1H), 3.40-3.36 (m, 1H), 2.18 (ddd, J=12.4, 8.4, 6.6 Hz, 1H), 2.14-2.02 (m, 1H), 1.94-1.76 (m, 1H), 1.75-1.61 (m, 1H), 1.51 (dddd, J=20.0, 15.4, 10.7, 6.6 Hz, 2H), 1.35-1.23 (m, 1H), 1.20 (t, J=7.1 Hz, 3H).

### Step 11: Preparation of Intermediate 151

Intermediate 15k (0.7 g), (E)-1-ethoxyvinyl-2-boronic acid pinacol ester (0.419 g), [1,1′-bis(diphenylphosphino)ferrocene]palladium dichloride dichloromethane complex (0.129 g), potassium carbonate (0.731 g), 1,4-dioxane (10 mL), and water (2 mL) were added to a microwave tube in sequence, N₂ was bubbled, and the mixture was microwaved at 120° C. and reacted for 2 h. The reaction solution was cooled to room temperature, and 100 mL of ethyl acetate and 100 mL of water were added. The organic phase was separated, dried over anhydrous sodium sulfate, filtered, and concentrated. The concentrate was separated and purified by silica gel column chromatography to give intermediate 151 (0.51 g).

MS(ESI, [M+H]⁺) m/z: 389.2.

### Step 12: Preparation of Intermediate 15m

Intermediate 151 (0.5 g), DCM (20 mL), hydrochloric acid (0.644 mL, 4 mol/L, 2.57 mmol) were added to a reaction flask in sequence, and the mixture was reacted at room temperature for 5 h. 100 mL of DCM and 100 mL of a saturated sodium bicarbonate solution were added to the system. The organic phase was separated, and the aqueous phase was then extracted 2 times with DCM (50 mL). The organic phases were combined, dried over anhydrous sodium sulfate, filtered and concentrated. The concentrate was separated and purified by silica gel column chromatography to give intermediate 15m (0.24 g).

MS(ESI, [M+H]⁺) m/z: 343.1

### Step 13: Preparation of Intermediate 15n

Intermediate 15m (0.18 g), acrylamide (0.041 g), and anhydrous tetrahydrofuran (10 mL) were added to a reaction flask in sequence. Potassium tert-butoxide (0.071 g) was slowly added at 0° C., and the mixture was reacted for 1 h. 50 mL of a saturated aqueous ammonium chloride solution was added to the reaction solution to quench the reaction, and the mixture was extracted with 50 mL of DCM. The organic phase was separated, dried over anhydrous sodium sulfate, filtered and concentrated. The concentrate was separated and purified by silica gel column chromatography to give intermediate 15n (0.095 g).

MS(ESI, [M+H]⁺) m/z: 368.2.

### Step 14: Preparation of Intermediate 15o

Intermediate 15n (0.09 g) and dichloromethane (5 mL) were added to a reaction flask, Dess-Martin periodinane (0.233 g) was added, and the mixture was reacted at room temperature for 1 h. After the reaction was completed, dichloromethane (50 mL) and water (50 mL) were added to the system. The organic phase was separated, and the aqueous phase was extracted 2 times with dichloromethane (50 mL). The organic phases were combined, dried over anhydrous sodium sulfate, filtered and concentrated to give intermediate 15o (0.08 g).

MS (ESI, [M−H]⁻) m/z: 364.1.

### Step 15: Preparation of Compound 15

Intermediate 15o (0.08 g), intermediate 1l (0.104 g), and dichloroethane (5 mL) were added to a reaction flask, 1 drop of acetic acid was added, and sodium cyanoborohydride (0.026 g) was then added. The mixture was reacted at room temperature for 2 h. After the reaction was completed, dichloromethane (50 mL) and water (50 mL) were added to the system. The organic phase was separated, and the aqueous phase was extracted 2 times with dichloromethane (50 mL). The organic phases were combined, dried over anhydrous sodium sulfate, filtered and concentrated. The concentrate was separated and purified by silica gel column chromatography to give compound 15 (0.042 g).

MS (ESI, [M+H]⁺) m/z: 828.4.

¹H NMR (500 MHz, DMSO-d₆) δ 11.18 (s, 2H), 7.91 (dd, J=9.2, 2.0 Hz, 1H), 7.80-7.69 (m, 2H), 7.66 (s, 1H), 7.56-7.42 (m, 3H), 7.37-7.29 (m, 1H), 7.15 (d, J=8.1 Hz, 2H), 6.58 (dd, J=7.9, 3.0 Hz, 1H), 5.09 (d, J=38.6 Hz, 1H), 4.69 (dd, J=11.9, 5.1 Hz, 1H), 4.31 (dd, J=38.9, 12.7 Hz, 2H), 3.60 (dq, J=10.9, 5.8, 4.5 Hz, 1H), 3.30 (d, J=7.9 Hz, 2H), 3.23 (d, J=8.1 Hz, 2H), 3.05-2.92 (m, 3H), 2.85 (ddd, J=17.4, 12.1, 5.5 Hz, 1H), 2.68 (s, 3H), 2.63 (dt, J=17.2, 4.2 Hz, 1H), 2.47-2.33 (m, 4H), 2.28-2.19 (m, 2H), 1.99 (s, 4H), 1.85-1.70 (m, 5H), 1.58 (d, J=36.0 Hz, 3H).

### Example 16: Synthesis of Compound 16

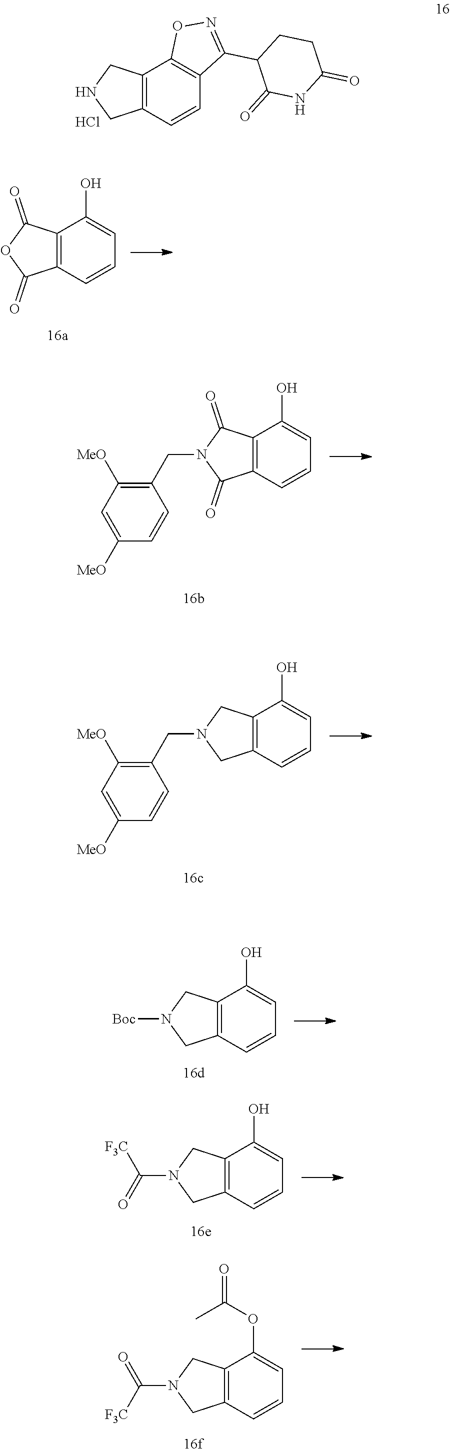

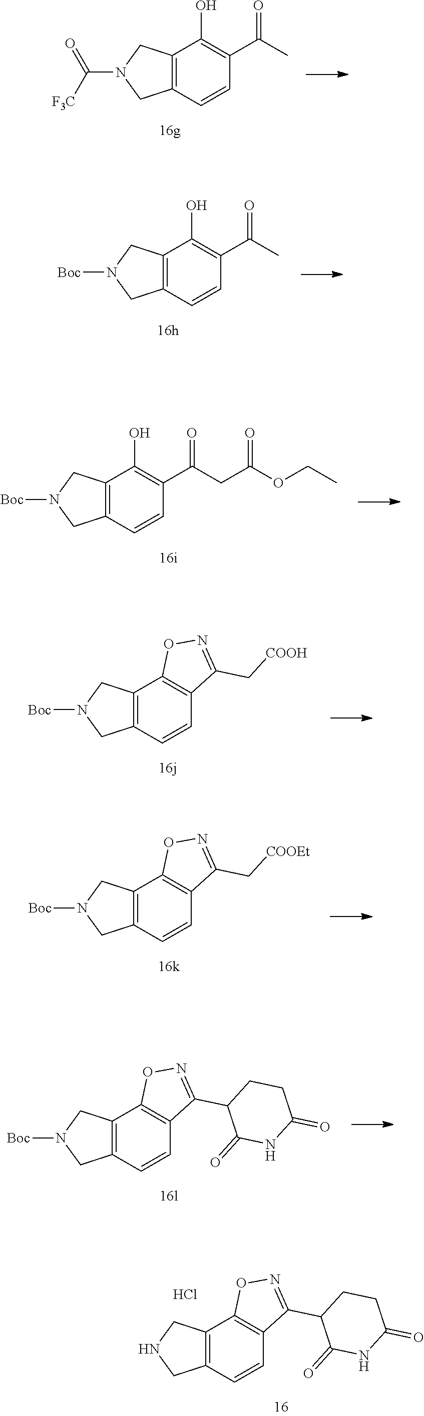

### Step 1: Preparation of Intermediate 16b

At 10 C, 16a (81 g), 2,4-dimethoxybenzylamine (83 g), and acetic acid (500 mL) were added to a reaction flask in sequence, and the mixture was warmed to 80° C. and reacted. After the reaction was completed, as confirmed by TLC, water was added to the reaction solution, and a solid was precipitated. Filter under vacuum was performed, and the filter cake was washed with water and dried to give (95.78 g) intermediate 16b.

MS (ESI, [M+H]⁻) m/z: 312.02

¹H NMR (500 MHz, DMSO-d₆) δ 11.03 (s, 1H), 7.62 (dd, J=8.4, 7.1 Hz, 1H), 7.29 (d, J=7.1 Hz, 1H), 7.22 (d, J=8.4 Hz, 1H), 6.90 (d, J=8.4 Hz, 1H), 6.56 (d, J=2.4 Hz, 1H), 6.43 (dd, J=8.4, 2.4 Hz, 1H), 4.60 (s, 2H), 3.80 (s, 3H), 3.73 (s, 3H).

### Step 2: Preparation of Intermediate 16c

At 10° C., a 2.5 M solution of lithium aluminum hydride in tetrahydrofuran (227 mL) was slowly added dropwise to a solution of 16b (96.00 g) in tetrahydrofuran (1000 mL), and the mixture was reacted at 80° C. After the reaction was completed, as confirmed by TLC, a 15 wt % aqueous sodium hydroxide solution and water were added to the reaction solution. Filter under vacuum was performed, the filter cake was washed with a dichloromethane:MeOH=1:1 solution, the filtrate was concentrated, and the crude product was separated and purified by silica gel column chromatography to give (55.87 g) intermediate 16c.

MS (ESI, [M+H]⁻) m/z: 286.01

¹H NMR (500 MHz, DMSO-d₆) δ 9.28 (s, 1H), 7.24 (d, J=8.3 Hz, 1H), 6.97 (t, J=7.7 Hz, 1H), 6.64 (d, J=7.4 Hz, 1H), 6.59 (d, J=8.0 Hz, 1H), 6.55 (d, J=2.4 Hz, 1H), 6.51 (dd, J=8.3, 2.4 Hz, 1H), 3.81-3.71 (m, 12H).

### Step 3: Preparation of Intermediate 16d

16c (48.00 g), methanol (350 mL), palladium hydroxide (4.8 g), and di-tert-butyl dicarbonate (41.4 g) were added to a reaction flask in sequence, and the mixture was reacted at 25° C. under hydrogen atmosphere. After the reaction was completed, as confirmed by TLC, the reaction solution was filtered under vacuum. The filtrate was concentrated, extracted with water and ethyl acetate, washed with saturated brine, dried over anhydrous sodium sulfate, and filtered. The filtrate was concentrated, and the crude product was separated by silica gel column chromatography and purified to give 34.43 g of intermediate 16d.

¹H NMR (500 MHz, DMSO-d₆) δ 7.07 (t, J=7.7 Hz, 1H), 6.68 (ddd, J=18.9, 7.8, 2.7 Hz, 2H), 4.56-4.50 (m, 2H), 4.48-4.42 (m, 2H), 1.45 (s, 9H).

### Step 4: Preparation of Intermediate 16e

16d (35.00 g), methanol (250 mL), and a solution of 4 M hydrochloric acid in dioxane (123 mL) were added to a reaction flask in sequence, and the mixture was reacted at 25° C. After the reaction was completed, as confirmed by TLC, the reaction solution was concentrated, pyridine (200 mL) and trifluoroacetic anhydride (25.20 g) were added, and the mixture was stirred and reacted at 25° C. After the reaction was completed, as confirmed by TLC, the reaction solution was poured into a 3 M aqueous hydrochloric acid solution, vigorously stirred, extracted with ethyl acetate, washed with saturated brine, dried over anhydrous sodium sulfate, and filtered. The filtrate was concentrated and dried to give 32.48 g of intermediate 16e.

MS (ESI, [M+H]⁻) m/z: 229.99

¹H NMR (500 MHz, DMSO-d₆) δ 9.82 (d, J=24.4 Hz, 1H), 7.16 (td, J=7.7, 3.7 Hz, 1H), 6.81 (dd, J=11.5, 7.5 Hz, 1H), 6.74 (d, J=8.0 Hz, 1H), 4.99 (s, 1H), 4.89 (s, 1H), 4.80 (s, 1H), 4.70 (s, 1H).

### Step 5: Preparation of Intermediate 16f

16e (32.48 g), dichloromethane (300 mL), triethylamine (28.40 g), 4-dimethylaminopyridine (1.72 g), and acetic anhydride (15.78 g) were added to a reaction flask in sequence, and the mixture was reacted at 25° C. After the reaction was completed, as confirmed by TLC, the reaction solution was poured into water, vigorously stirred, extracted with dichloromethane, washed with saturated brine, dried over anhydrous sodium sulfate, and filtered. The filtrate was concentrated, and the crude product was separated by silica gel column chromatography and purified to give 33.82 g net weight of intermediate 16f.

¹H NMR (500 MHz, DMSO-d₆) δ 7.41 (td, J=7.8, 2.3 Hz, 1H), 7.30 (t, J=8.5 Hz, 1H), 7.12 (dd, J=7.9, 2.9 Hz, 1H), 5.09 (s, 1H), 4.90 (d, J=12.3 Hz, 2H), 4.72 (s, 1H), 2.31 (d, J=3.4 Hz, 3H).

### Step 6: Preparation of Intermediate 16g

16f (30.00 g), aluminum trichloride (29.30 g), and orthodichlorobenzene (200 mL) were added to a reaction flask in sequence, and the mixture was reacted at 150° C. After the reaction was completed, as confirmed by TLC, the reaction solution was poured into an aqueous citric acid solution, vigorously stirred, extracted with ethyl acetate, washed with saturated brine, dried over anhydrous sodium sulfate, and filtered. The filtrate was concentrated, and the crude product was separated by silica gel column chromatography and purified to give (18.19 g) net weight of intermediate 16g.

¹H NMR (500 MHz, DMSO-d₆) δ 12.38 (s, 1H), 7.94 (dd, J=8.2, 4.7 Hz, 1H), 7.01 (dd, J=12.7, 8.1 Hz, 1H), 5.07 (s, 1H), 4.94 (s, 1H), 4.86 (s, 1H), 4.74 (d, J=1.6 Hz, 1H), 2.66 (d, J=1.0 Hz, 3H).

### Step 7: Preparation of Intermediate 16h

16g (18.19 g), sodium hydroxide (7.56 g), methanol (150 mL), and water (150 mL) were added to a reaction flask in sequence, and the mixture was reacted at 25° C. for 2.5 h. The reaction solution was concentrated to remove methanol, 1,4-dioxane (150 mL) and di-tert-butyl dicarbonate (15.14 g) were added to the residue, and the mixture was stirred and reacted at 25° C. for 2 h. The reaction solution was poured into water, vigorously stirred, extracted with ethyl acetate, washed with saturated brine, dried over anhydrous sodium sulfate, and filtered. The filtrate was concentrated, and the crude product was separated by silica gel column chromatography and purified to give (14.10 g) intermediate 16h.

MS (ESI, [M+H]⁻) m/z: 276.07

¹H NMR (500 MHz, DMSO-d₆) δ 12.36 (s, 1H), 7.89 (dd, J=8.1, 2.9 Hz, 1H), 6.95 (t, J=8.0 Hz, 1H), 4.62 (dt, J=13.4, 2.2 Hz, 2H), 4.51 (dt, J=13.9, 2.3 Hz, 2H), 2.65 (s, 3H), 1.46 (s, 9H).

### Step 8: Preparation of Intermediate 161

16h (14.10 g), diethyl carbonate (27.00 g), and toluene (200 mL) were added to a reaction flask in sequence, and the mixture was cooled to 0° C. After 60 wt % sodium hydride (9.16 g) was added, the mixture was warmed to 120° C., stirred, and reacted. After the reaction was completed, as confirmed by TLC, the reaction solution was poured into a 3 M aqueous hydrochloric acid solution, vigorously stirred, extracted with ethyl acetate, washed with saturated brine, dried over anhydrous sodium sulfate, and filtered. The filtrate was concentrated, and the crude product was separated by silica gel column chromatography and purified to give (14.96 g) net weight of intermediate 161.

MS (ESI, [M+H]⁺) m/z: 250.20

¹H NMR (500 MHz, DMSO-d₆) δ 11.72 (d, J=9.6 Hz, 1H), 7.82 (dd, J=8.2, 4.7 Hz, 1H), 6.97 (t, J=8.4 Hz, 1H), 4.63 (dt, J=12.4, 2.1 Hz, 2H), 4.53 (dt, J=13.7, 2.1 Hz, 2H), 4.22 (d, J=2.5 Hz, 2H), 4.13 (q, J=7.1 Hz, 2H), 1.46 (d, J=1.9 Hz, 9H), 1.19 (t, J=7.1 Hz, 3H).

### Step 9: Preparation of Intermediate 16j

16i (14.96 g), a 50% aqueous hydroxylamine solution (6.36 g), and ethanol (150 mL) were added to a reaction flask in sequence, and the mixture was stirred and reacted at 85° C. After the reaction was completed, as confirmed by TLC, the reaction solution was concentrated, and water and ethyl acetate were then added. The mixture was extracted and separated, and the organic phase was washed with saturated brine, dried over anhydrous sodium sulfate, and filtered. The filtrate was concentrated to give a crude product of intermediate 16j (10.13 g).

MS (ESI, [M+H]⁻) m/z: 317.20

¹H NMR (500 MHz, DMSO-d₆) δ 12.59 (s, 1H), 7.78 (d, J=8.2 Hz, 1H), 7.38 (t, J=7.6 Hz, 1H), 4.85 (dt, J=13.5, 2.5 Hz, 2H), 4.75 (dt, J=12.2, 2.4 Hz, 2H), 4.11 (s, 2H), 1.49 (d, J=2.1 Hz, 9H).

### Step 10: Preparation of Intermediate 16k

16j (10.13 g), potassium carbonate (12.55 g), N,N-dimethylacetamide (150 mL), and iodoethane (7.08 g) were added to a reaction flask in sequence, and the mixture was stirred and reacted at 80° C. After the reaction was completed, as confirmed by TLC, the reaction solution was poured into water, vigorously stirred, extracted with ethyl acetate, washed with saturated brine, dried over anhydrous sodium sulfate, and filtered. The filtrate was concentrated, and the crude product was separated by silica gel column chromatography and purified to give (11.22 g) net weight of intermediate 16k.

MS (ESI, [M+H]⁺) m/z: 247.16

¹H NMR (500 MHz, DMSO-d₆) δ 7.78 (d, J=8.0 Hz, 1H), 7.39 (t, J=7.5 Hz, 1H), 4.89-4.82 (m, 2H), 4.75 (dd, J=11.8, 2.8 Hz, 2H), 4.22 (d, J=4.3 Hz, 2H), 4.13 (p, J=7.2 Hz, 2H), 1.48 (d, J=2.1 Hz, 9H), 1.21-1.17 (m, 3H).

### Step 11: Preparation of Intermediate 161

Acrylamide (1.32 g) was slowly added to a stirred solution of 16k (10.72 g) in tetrahydrofuran (50 mL) at 0° C. under N₂ atmosphere, a 1 M solution of potassium tert-butoxide in tetrahydrofuran (18.63 mL) was then added dropwise, and the mixture was stirred and reacted at 0° C. After the reaction was completed, as confirmed by TLC, the reaction solution was poured into an aqueous ammonium chloride solution, vigorously stirred, extracted with ethyl acetate, washed with saturated brine, dried over anhydrous sodium sulfate, and filtered. The filtrate was concentrated, and the crude product was separated by silica gel column chromatography and purified to give (7.52 g) net weight of intermediate 161.

MS (ESI, [M+H]⁺) m/z: 272.02

¹H NMR (500 MHz, DMSO-d₆) δ 11.11 (s, 1H), 7.80 (dd, J=8.1, 2.7 Hz, 1H), 7.37 (t, J=7.9 Hz, 1H), 4.85 (dd, J=12.2, 2.6 Hz, 2H), 4.75 (dt, J=12.0, 2.3 Hz, 2H), 4.63 (dd, J=12.1, 4.9 Hz, 1H), 2.78 (ddd, J=17.3, 12.1, 5.3 Hz, 1H), 2.67-2.51 (m, 2H), 2.20 (dq, J=13.5, 4.7 Hz, 1H), 1.49 (d, J=2.6 Hz, 9H).

### Step 12: Preparation of Compound 16

16l (1.00 g), a solution of 4 M hydrochloric acid in dioxane (10 mL), and ethyl acetate (50 mL) were added to a reaction flask in sequence, and the mixture was reacted at 25° C. After the reaction was completed, as confirmed by TLC, the reaction solution was directly filtered, and the filter cake was washed with ethyl acetate and dried to give 0.83 g of compound 16.

MS (ESI, [M+H]⁺) m/z: 272.11

¹H NMR (500 MHz, DMSO-d₆) δ 11.13 (s, 1H), 10.36 (s, 2H), 7.90 (d, J=8.1 Hz, 1H), 7.45 (d, J=8.2 Hz, 1H), 4.81 (s, 2H), 4.70-4.63 (m, 3H), 2.79 (ddd, J=17.4, 12.2, 5.3 Hz, 1H), 2.62 (dt, J=17.3, 4.0 Hz, 1H), 2.59-2.52 (m, 1H), 2.21 (ddt, J=13.2, 5.1, 2.5 Hz, 1H).

### Example 17: Preparation of Compound 17

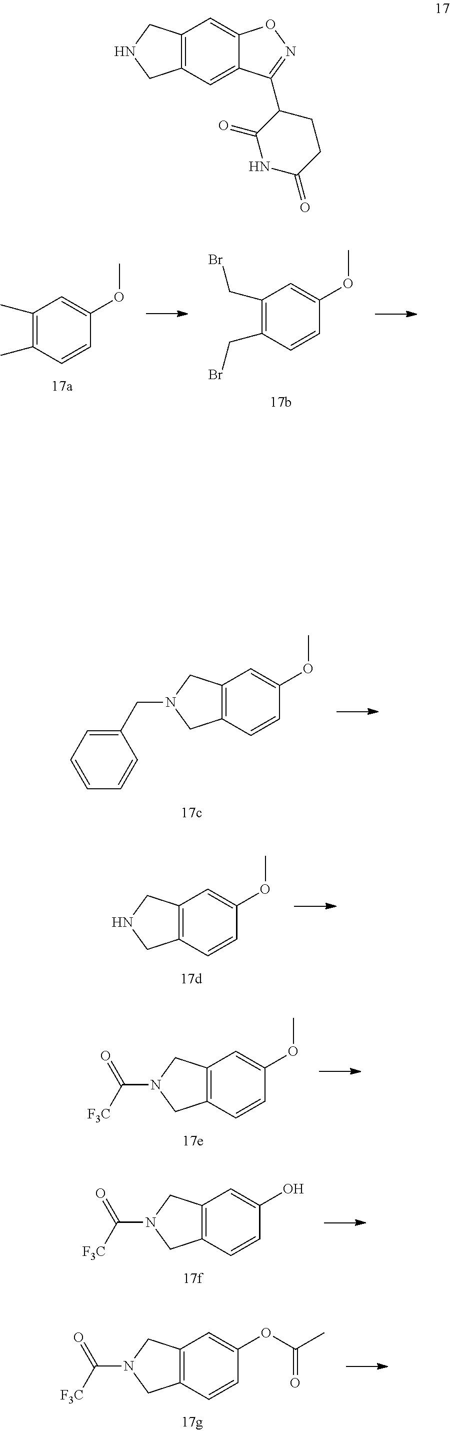

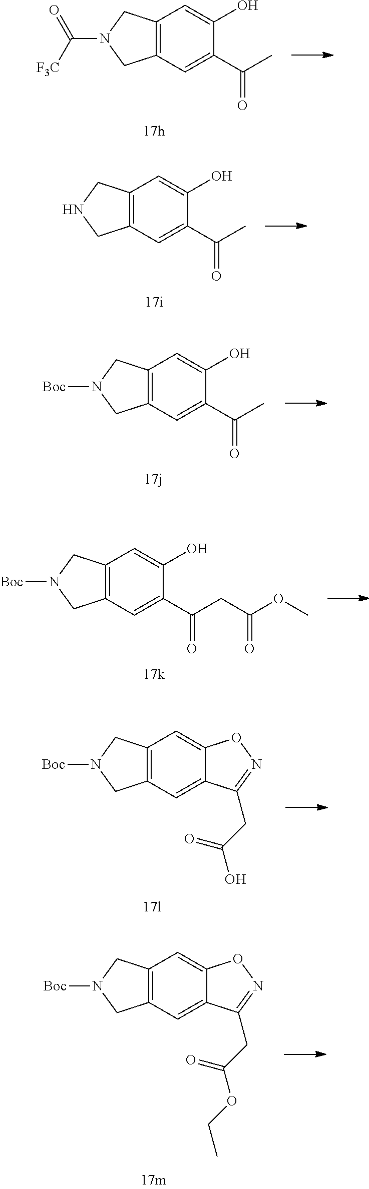 [SMILES: CC(=O)c1cc2c(cc1O)CN(C(=O)C(F)(F)F)C2 ; CC(=O)c1cc2c(cc1O)CNC2 ; CC(=O)c1cc2c(cc1O)CN(C(=O)OC(C)(C)C)C2 ; COC(=O)CC(=O)c1cc2c(cc1O)CN(C(=O)OC(C)(C)C)C2 ; CC(C)(C)OC(=O)N1Cc2cc3onc(CC(=O)O)c3cc2C1 ; CCOC(=O)Cc1noc2cc3c(cc12)CN(C(=O)OC(C)(C)C)C3]

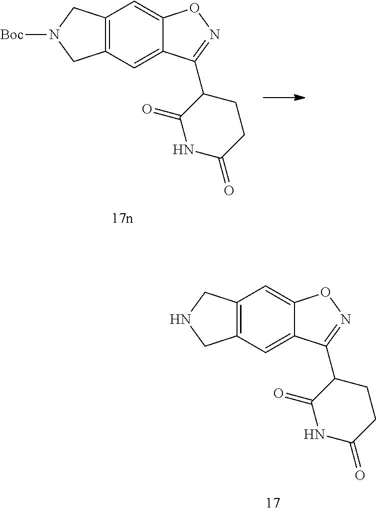 [SMILES: CC(C)(C)OC(=O)N1Cc2cc3onc(C4CCC(=O)NC4=O)c3cc2C1 ; O=C1CCC(c2noc3cc4c(cc23)CNC4)C(=O)N1]

### Step 1: Preparation of Intermediate 17b

17a (18 g), AIBN (0.738 g), carbon tetrachloride (500 mL), and NBS (47.8 g) were added to a reaction flask in sequence, and the mixture was warmed to 60° C. and reacted. After the reaction was completed, as confirmed by TLC, the reaction solution was cooled to room temperature and concentrated by evaporation at reduced pressure to remove the solvent, and dichloromethane was added to the residue. The mixture was washed with saturated brine, dried over anhydrous sodium sulfate, and filtered. The filtrate was concentrated by evaporation at reduced pressure to remove the solvent and purified by silica gel column chromatography to give intermediate 17b (18.7 g).

### Step 2: Preparation of Intermediate 17c

17b (18.7 g), benzylamine (1.78 mL), N,N-diisopropylethylamine (7.13 mL), and toluene (50 mL) were added to a reaction flask in sequence, and the mixture was warmed to 50° C. and reacted. After the reaction was completed, as confirmed by TLC, the reaction solution was cooled to room temperature. After the reaction was completed, the stirring was stopped, and the reaction was carried out at room temperature for a total of 2 h. Ethyl acetate and 1 M solution of HCl in ice water were added to the reaction solution for extraction. The aqueous phase was collected, the pH was adjusted to about 9 with sodium bicarbonate solid, and ethyl acetate was added for extraction. The organic phase was collected, dried over anhydrous sodium sulfate, filtered, and concentrated by evaporation at reduced pressure to remove the solvent, thus giving intermediate 17c (9.6 g).

MS(ESI, [M+H]⁺) m/z: 240.1.

### Step 3: Preparation of Intermediate 17d

17c (9.6 g), methanol (200 mL), 10% palladium on carbon (5 g), and toluene (50 mL) were added to a reaction flask in sequence, and the mixture was purged 3 times with hydrogen and reacted at room temperature under hydrogen atmosphere. The mixture was reacted at room temperature. After the reaction was completed, as confirmed by TLC, the palladium on carbon was filtered, and the filter cake was washed 2 times with methanol. The filtrate was collected and concentrated by evaporation at reduced pressure to remove the solvent, thus giving 17d (4.5 g).

MS(ESI, [M+H]⁺) m/z: 149.9.

### Step 4: Preparation of Intermediate 17e

17d (4 g), tetrahydrofuran (50 mL), and trifluoroacetic anhydride (5.63 g) were added to a reaction flask in sequence, and the mixture was reacted at room temperature. After the reaction was completed, as confirmed by TLC, an aqueous solution was added to the reaction solution to quench the reaction, and ethyl acetate was then added for extraction. The organic phase was separated, dried over anhydrous sodium sulfate, and purified by silica gel column chromatography to give 17e (4.58 g).

¹H NMR (500 MHz, DMSO-d6) δ 7.29 (t, J=9.3 Hz, 1H), 6.98 (d, J=8.6 Hz, 1H), 6.93-6.86 (m, 1H), 4.97 (d, J=21.7 Hz, 2H), 4.77 (dd, J=21.4, 5.2 Hz, 2H), 3.76 (dd, J=3.6, 1.6 Hz, 3H).

### Step 5: Preparation of Intermediate 17f

17e (4.6 g), dichloromethane (200 mL), and boron tribromide (1 M, 18.76 mL) were added to a reaction flask in sequence, and the mixture was reacted at room temperature. After the reaction was completed, as confirmed by TLC, water was added to the reaction solution under an ice bath to quench the reaction. The organic phase was separated, and the aqueous phase was extracted with dichloromethane. The organic phases were combined, dried over anhydrous sodium sulfate, and concentrated by evaporation at reduced pressure to remove the solvent, thus giving 17f (4.2 g).

¹H NMR (500 MHz, DMSO-d6) δ 7.16 (t, J=8.8 Hz, 1H), 6.87-6.59 (m, 2H), 4.92 (d, J=20.8 Hz, 2H), 4.72 (d, J=19.9 Hz, 2H).

### Step 6: Preparation of Intermediate 17g

17f (6.5 g), dichloromethane (60 mL), triethylamine (7.8 mL), and acetic anhydride (2.94 mL) were added to a reaction flask in sequence, and the mixture was reacted at room temperature. After the reaction was completed, as confirmed by TLC, dichloromethane and water were added to the reaction solution. The organic phase was separated, washed with saturated brine, dried over anhydrous sodium sulfate, filtered, and concentrated by evaporation at reduced pressure to remove the solvent, thus giving 17g (7 g).

¹H NMR (500 MHz, DMSO-d6) δ 7.42 (dd, J=10.0, 8.3 Hz, 1H), 7.17 (dd, J=14.0, 2.1 Hz, 1H), 7.09 (d, J=8.2 Hz, 1H), 5.03 (d, J=6.6 Hz, 2H), 4.83 (d, J=6.9 Hz, 2H), 2.27 (s, 3H).

### Step 7: Preparation of Intermediate 17h

17g (6 g) and aluminum trichloride (4.39 g) were added to a reaction flask in sequence, and the mixture was gradually heated from room temperature to 150° C. and reacted. After the reaction was completed, as confirmed by TLC, the reaction solution was cooled to room temperature, and water and a 3 M aqueous hydrochloric acid solution were added to the residue. The organic phase was separated, washed with saturated brine, dried over anhydrous sodium sulfate, and filtered. The filtrate was concentrated under reduced pressure to remove the solvent and purified by silica gel column chromatography to give 17h (3.78 g).

MS(ESI, [M−H]⁻) m/z: 271.9.

### Step 7: Preparation of Intermediate 17j

17h (550 mg), MeOH (5.00 mL), and an aqueous solution (5.00 mL) of sodium hydroxide (242 mg) were added to a reaction flask in sequence, and the mixture was reacted at room temperature. After the reaction was completed, as confirmed by TLC, the reaction solution was concentrated to remove methanol, and the aqueous phase was retained. 171 was obtained. 1,4-Dioxane (5 mL) and Boc anhydride (439 mg, 0.462 mL) were added to the system, and the mixture was reacted at room temperature. After the reaction was completed, as confirmed by TLC, the reaction solution was extracted with ethyl acetate and saturated brine, and the organic phase was dried over anhydrous sodium sulfate, filtered, and concentrated to give 17j (200 mg).

¹H NMR (500 MHz, DMSO-d6) δ 12.11 (d, J=19.3 Hz, 1H), 7.88 (d, J=9.5 Hz, 1H), 6.74-6.56 (m, 1H), 4.60-4.50 (m, 4H), 2.63 (d, J=6.5 Hz, 3H), 1.45 (s, 9H).

### Step 8: Preparation of Intermediate 17k

17j (3.5 g) and THF (300 mL) were added to a reaction flask in sequence, and diethyl carbonate (14.91 g, 15.29 mL) was added. The mixture was cooled to about 0° C., and 60 wt % sodium hydride (5.05 g, 126 mmol) was added in portions. The mixture was heated to 85° C. and reacted. After the reaction was completed, as confirmed by TLC, the reaction solution was cooled to room temperature and slowly poured into ice water. The mixture was extracted with ethyl acetate, and the organic phase was discarded. The aqueous phase was adjusted to pH=1-2 with 3 M hydrochloric acid, then extracted with ethyl acetate, dried over anhydrous sodium sulfate, filtered, and concentrated by evaporation at reduced pressure to remove the solvent, thus giving 17k (7.0 g).

MS(ESI, [M−H]⁻) m/z: 348.3.

### Step 9: Preparation of Intermediate 171

17k (4.4 g), an aqueous hydroxylamine solution (4.16 g, 63.0 mmol), and ethanol (50 mL) were added to a reaction flask in sequence, and the mixture was reacted at 85° C. After the reaction was completed, as confirmed by TLC, the reaction solution was cooled to room temperature, ethyl acetate and a saturated aqueous sodium carbonate solution were added to the residue for extraction, and the organic phase was discarded. The aqueous phase was adjusted to pH=2-3 with a 1 M aqueous HCl solution, extracted with ethyl acetate, dried over anhydrous sodium sulfate, filtered, and concentrated by evaporation at reduced pressure to remove the solvent, thus giving 171 (3.25 g).

¹H NMR (500 MHz, DMSO-d₆) δ 12.83 (s, 1H), 7.74 (d, J=13.0 Hz, 1H), 7.68 (d, J=3.5 Hz, 1H), 4.71 (d, J=13.6 Hz, 2H), 4.66 (d, J=11.4 Hz, 2H), 4.07 (s, 2H), 1.47 (s, 9H).

### Step 10: Preparation of Intermediate 17m

17l (3.14 g), potassium carbonate (1.500 g), DMA (5 mL), and iodoethane (2.308 g, 1.183 mL) were added to a reaction flask in sequence, and the mixture was heated to 80° C. and reacted. After the reaction was completed, as confirmed by TLC, the reaction solution was cooled to room temperature and poured into a mixed solution of ethyl acetate and water. The organic phase was separated, dried over anhydrous sodium sulfate, and filtered. The filtrate was concentrated by evaporation at reduced pressure to remove the solvent, thus giving 17m (2.47 g).

¹H NMR (500 MHz, DMSO-d₆) δ 7.73 (d, J=15.9 Hz, 1H), 7.69 (d, J=3.5 Hz, 1H), 4.69 (dd, J=23.8, 12.4 Hz, 4H), 4.19-4.11 (m, 4H), 1.47 (s, 9H), 1.20 (t, J=7.1 Hz, 3H).

### Step 11: Preparation of Intermediate 17n

17m (1.5 g) and THF (75 mL) were added to a reaction flask in sequence, acrylamide (0.215 g) was added, and the mixture was cooled to about −15° C. A 1 M solution of potassium tert-butoxide in tetrahydrofuran (2.60 mL) was added, and the system was warmed to 0° C. and reacted. After the reaction was completed, as confirmed by TLC, the mixture was added dropwise to an ammonium chloride solution to quench the reaction and extracted with ethyl acetate. The organic phase was separated, dried over anhydrous sodium sulfate, and filtered, and the filtrate was concentrated and purified by silica gel column chromatography to give 17n (0.88 g).

¹H NMR (500 MHz, DMSO-d₆) δ 11.11 (d, J=3.4 Hz, 1H), 7.78 (d, J=12.4 Hz, 1H), 7.70 (s, 1H), 4.68 (dd, J=29.4, 13.3 Hz, 4H), 4.58 (dd, J=12.0, 4.9 Hz, 1H), 2.79 (ddd, J=17.3, 12.1, 5.3 Hz, 1H), 2.62 (dt, J=17.3, 4.1 Hz, 1H), 2.56-2.50 (m, 1H), 2.31-2.14 (m, 1H), 1.47 (d, J=1.5 Hz, 9H).

### Step 12: Preparation of Compound 17

17n (0.428 g) and dichloromethane (10.00 mL) were added to a reaction flask in sequence, then trifluoroacetic acid (3.29 g, 2.211 mL) was added, and the mixture was reacted at room temperature. After the reaction was completed, as confirmed by TLC, water was added to the reaction solution, and the pH was adjusted to 7-8 with saturated sodium bicarbonate. The mixture was extracted with dichloromethane, dried over anhydrous sodium sulfate, and subjected to rotary evaporation to remove the solvent, thus giving 17 (0.439 g).

MS(ESI, [M+H]⁺) m/z: 272.24.

¹H NMR (500 MHz, DMSO-d6) δ 7.81-7.77 (m, 1H), 7.74 (s, 1H), 4.59 (dd, J=12.1, 5.0 Hz, 1H), 4.49 (s, 2H), 4.43 (s, 2H), 2.79 (ddd, J=17.4, 12.2, 5.3 Hz, 1H), 2.62 (dt, J=17.3, 4.0 Hz, 1H), 2.47 (dd, J=12.4, 4.4 Hz, 1H), 2.20 (ddt, J=13.3, 5.2, 2.6 Hz, 1H).

### Example 18: Synthesis of Compound 18

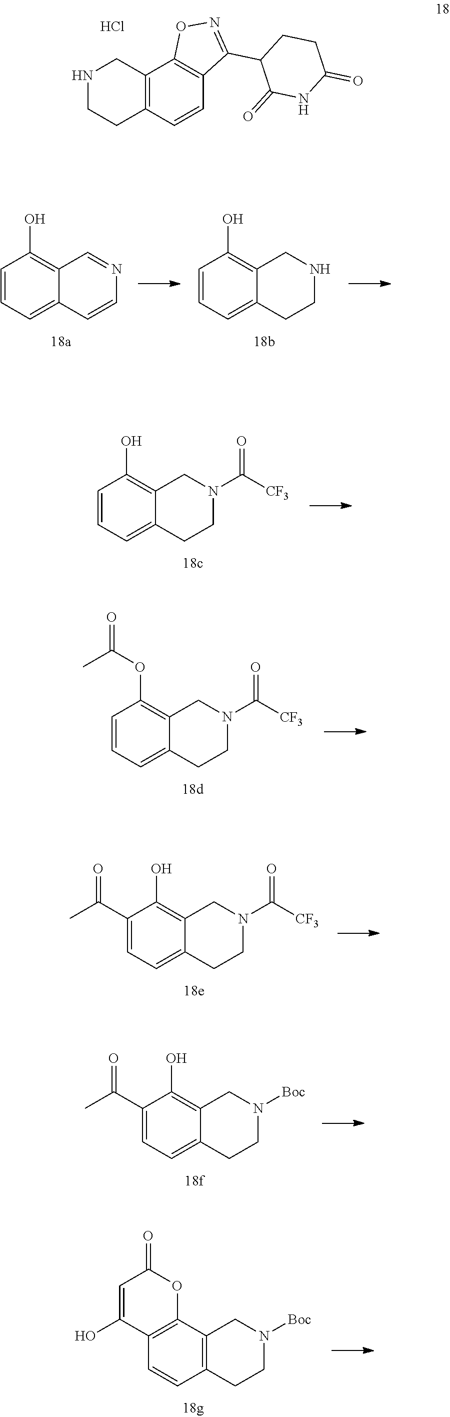

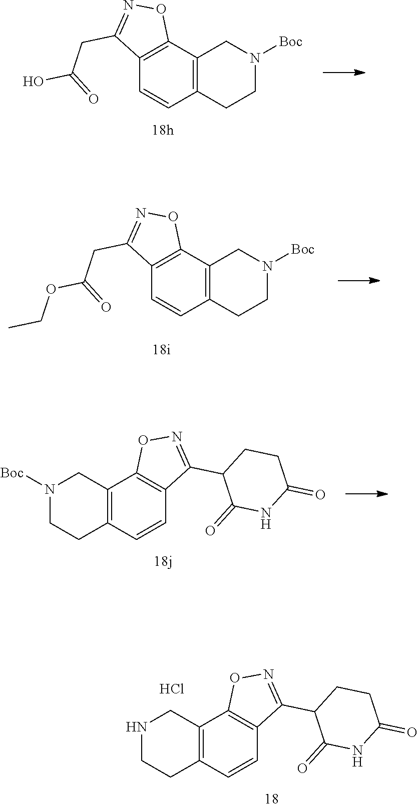 [SMILES: CC(C)(C)OC(=O)N1CCc2ccc3c(CC(=O)O)noc3c2C1 ; CCOC(=O)Cc1noc2c3c(ccc12)CCN(C(=O)OC(C)(C)C)C3 ; CC(C)(C)OC(=O)N1CCc2ccc3c(C4CCC(=O)NC4=O)noc3c2C1 ; O=C1CCC(c2noc3c4c(ccc23)CCNC4)C(=O)N1 ; Cl]

### Step 1: Preparation of Intermediate 18b

Intermediate 18a (25 g) was completely dissolved in methanol (1000 mL) and acetic acid (103 g, 99 mL) and then added to a high pressure reactor under a hydrogen pressure of 3 Mpa and a temperature of 110° C. After the reaction was completed, as confirmed by TLC, the reaction solution was concentrated by evaporation at reduced pressure to remove the solvent, and a solution of hydrochloric acid in dioxane (4 mol/L, 100 mL, 400 mmol) was added to the residue. The mixture was concentrated by evaporation at reduced pressure to remove the solvent, the residue was slurried with ethyl acetate and filtered, and the filter cake was collected to give the target intermediate 18b (28.97 g).

MS(ESI, [M+H]⁺) m/z: 150.0.

1H NMR (500 MHz, DMSO-d6) δ 10.01 (s, 1H), 7.06 (t, J=7.8 Hz, 1H), 6.76 (d, J=8.0 Hz, 1H), 6.64 (d, J=7.6 Hz, 1H), 4.01 (t, J=4.9 Hz, 2H), 3.33-3.25 (m, 2H), 2.94 (t, J=6.2 Hz, 2H).

### Step 2: Preparation of Intermediate 18c

Intermediate 18b (28.97 g) and tetrahydrofuran (300 mL) were added to a reaction flask in sequence, and trifluoroacetic anhydride (27.0 mL) was added under an ice bath. The mixture was reacted at room temperature. After the reaction was completed, as confirmed by TLC, the reaction solution was extracted with ethyl acetate and water. The organic phase was separated, and the aqueous phase was extracted twice with ethyl acetate. The organic phases were combined, washed with saturated brine, dried over anhydrous sodium sulfate, filtered, and concentrated by evaporation at reduced pressure to remove the solvent, and the filter cake was collected by filtration to give the target intermediate 18c (22.67 g).

MS(ESI, [M+H]⁻) m/z: 244.0.

¹H NMR (500 MHz, DMSO-d6) δ 7.03 (q, J=7.9 Hz, 1H), 6.74-6.68 (m, 1H), 6.64 (t, J=6.9 Hz, 1H), 4.61 (d, J=23.5 Hz, 2H), 3.78 (td, J=6.0, 3.7 Hz, 2H), 2.84 (dt, J=16.8, 5.9 Hz, 2H).

### Step 3: Preparation of Intermediate 18d

Intermediate 18c (22.67 g), dichloromethane (200 mL), triethylamine (28.1 g, 38.6 mL), and DMAP (0.282 g, 2.311 mmol) were added to a reaction flask in sequence, and acetic anhydride (10.38 g, 9.68 mL) was added under an ice bath. The mixture was allowed to return to room temperature and react. After the reaction was completed, as confirmed by TLC, the reaction solution was concentrated by evaporation at reduced pressure to remove the solvent, and the residue was extracted with ethyl acetate and water. The organic phase was separated, washed with a saturated ammonium chloride solution and saturated brine, dried over anhydrous sodium sulfate, and filtered, and the filtrate was concentrated by evaporation at reduced pressure to remove the solvent, thus giving the target intermediate 18d (21.94 g).

¹H NMR (500 MHz, DMSO-d6) δ 7.30 (dt, J=11.1, 7.8 Hz, 1H), 7.18-7.11 (m, 1H), 7.05 (dt, J=8.0, 2.2 Hz, 1H), 4.59 (s, 2H), 3.81 (q, J=6.1 Hz, 2H), 2.95 (dt, J=10.1, 6.0 Hz, 2H), 2.33 (d, J=9.5 Hz, 3H).

### Step 4: Preparation of Intermediate 18e

Intermediate 18d (21 g) and aluminum trichloride (14.62 g) were added to a reaction flask in sequence and the mixture was heated to 170° C. and reacted under N₂ atmosphere. After the reaction was completed, as confirmed by TLC, the reaction solution was cooled to room temperature, water was added to quench the reaction, and dichloromethane was added for extraction. The organic phase was separated, washed with 500 mL of saturated brine, dried over anhydrous sodium sulfate, and filtered, and the filtrate was purified by silica gel column chromatography to give the target intermediate 18e (10.16 g).

MS(ESI, [M+H]⁻) m/z: 286.0.

¹H NMR (500 MHz, DMSO-d6) δ 12.76 (d, J=8.4 Hz, 1H), 7.83 (t, J=8.8 Hz, 1H), 6.86 (dd, J=8.3, 5.7 Hz, 1H), 4.67 (d, J=25.1 Hz, 2H), 3.86-3.78 (m, 2H), 2.94 (dt, J=13.3, 5.9 Hz, 2H), 2.64 (d, J=1.2 Hz, 3H).

### Step 5: Preparation of Intermediate 18f

Intermediate 18e (10.16 g) and MeOH (100 mL) were added to a reaction flask in sequence, and a solution of sodium hydroxide (4.24 g) in water (100 mL) was added dropwise under an ice bath. The mixture was allowed to return to room temperature and react. After the reaction was completed, as confirmed by TLC, the reaction solution was concentrated by evaporation at reduced pressure to remove methanol, and 1,4-dioxane (100 mL) and di-tert-butyl dicarbonate (8.49 g, 9.03 mL) were added. The mixture was allowed to return to room temperature and react. After the reaction was completed, as confirmed by TLC, the reaction solution was extracted with ethyl acetate and water. The organic phase was separated, washed with 1000 mL of saturated brine, dried over anhydrous sodium sulfate, and filtered, and the filtrate was concentrated by evaporation at reduced pressure to remove the solvent, thus giving the target intermediate 18f (8.96 g).

MS(ESI, [M+H]⁺) m/z: 192.0.

¹H NMR (500 MHz, DMSO-d6) δ 12.72 (s, 1H), 7.76 (d, J=8.2 Hz, 1H), 6.80 (d, J=8.2 Hz, 1H), 4.41 (s, 2H), 3.55 (t, J=5.8 Hz, 2H), 2.80 (t, J=5.8 Hz, 2H), 2.63 (s, 3H), 1.43 (s, 9H).

### Step 6: Preparation of Intermediate 18g

Intermediate 18f (8.76 g), diethyl carbonate (17.76 g, 18.21 mL), and toluene (90 mL) were added to a reaction flask in sequence, 60 wt % sodium hydride (6.01 g, 150 mmol) was added in portions under an ice bath. The mixture was heated to 120° C. and reacted. After the reaction was completed, as confirmed by TLC, the reaction solution was cooled to room temperature and poured into ice water to quench the reaction. The pH was adjusted to 1-2 with a 1 M hydrochloric acid solution, and ethyl acetate was added for extraction. The organic phase was separated, washed with 600 mL of saturated brine, dried over anhydrous sodium sulfate, and filtered, and the filtrate was concentrated by evaporation at reduced pressure to remove the solvent. The residue was slurried with petroleum ether and filtered, and the filter cake was collected to give the target intermediate 18g (12.03 g).

### Step 7: Preparation of Intermediate 18h

Intermediate 18g (9.54 g), EtOH (100 mL), and an aqueous hydroxylamine solution (9.93 g, 9.21 mL, 150 mmol) were added to a reaction flask in sequence, and the mixture was heated to 85° C. and reacted. After the reaction was completed, as confirmed by TLC, a saturated sodium carbonate solution was added to the reaction solution to adjust the pH to 8, and ethyl acetate was added for extraction. The organic phase was separated and extracted twice with water. Then the organic phases were combined, the pH was adjusted to 3 with 1 M hydrochloric acid, and ethyl acetate was added for extraction three times. The organic phases were combined, washed with saturated brine, dried over anhydrous sodium sulfate, and filtered, and the filtrate was concentrated by evaporation at reduced pressure to remove the solvent, thus giving the target intermediate 18h (9.01 g).

MS(ESI, [M+H]⁻) m/z: 331.0.

### Step 8: Preparation of Intermediate 181

Intermediate 18h (9.01 g), potassium carbonate (11.24 g), DMA (90 mL), and iodoethane (5.07 g, 2.63 mL, 32.5 mmol) were added to a reaction flask in sequence, and the mixture was heated to 80° C. and reacted under N₂ atmosphere. After the reaction was completed, as confirmed by TLC, the reaction solution was cooled to room temperature and extracted with ethyl acetate and water. The organic phase was separated, washed with saturated brine, dried over anhydrous sodium sulfate, and filtered, and the filtrate was purified by silica gel column chromatography to give the target intermediate 181 (7.33 g).

MS(ESI, [M+H]⁺) m/z: 361.0.

¹H NMR (500 MHz, DMSO-d6) δ 7.63 (d, J=8.1 Hz, 1H), 7.21 (d, J=8.2 Hz, 1H), 4.76 (s, 2H), 4.18 (s, 2H), 4.13 (q, J=7.1 Hz, 2H), 3.66 (t, J=5.8 Hz, 2H), 2.93 (t, J=5.8 Hz, 2H), 1.45 (s, 9H), 1.19 (t, J=7.1 Hz, 3H).

### Step 9: Preparation of Intermediate 18j

Intermediate 181 (7.3 g), tetrahydrofuran (80 mL), and acrylamide (0.864 g, 12.15 mmol) were added to a reaction flask in sequence, the mixture was cooled to −15° C. under N₂ atmosphere, and then a solution of potassium tert-butoxide in tetrahydrofuran (1 mol/L, 11.14 mL, 11.14 mmol) was added dropwise. After the dropwise addition was completed, the mixture was warmed to 0° C. and reacted. After the reaction was completed, as confirmed by TLC, the system was added to a saturated ammonium chloride solution and extracted with ethyl acetate. The organic phase was separated, dried over anhydrous sodium sulfate, and filtered, and the filtrate was purified by silica gel column chromatography to give the target intermediate 18j (4.54 g).

MS(ESI, [M+H]⁻) m/z: 384.3

¹H NMR (500 MHz, DMSO-d6) δ 11.09 (s, 1H), 7.65 (d, J=8.1 Hz, 1H), 7.20 (d, J=8.2 Hz, 1H), 4.76 (s, 2H), 4.58 (dd, J=12.0, 5.0 Hz, 1H), 3.66 (t, J=5.8 Hz, 2H), 2.93 (t, J=5.8 Hz, 2H), 2.77 (ddd, J=17.3, 12.1, 5.3 Hz, 1H), 2.61 (dt, J=17.3, 4.1 Hz, 1H), 2.54 (d, J=4.5 Hz, 1H), 2.18 (dtd, J=13.5, 5.2, 3.6 Hz, 1H), 1.45 (s, 9H).

### Step 10: Preparation of Compound 18

Intermediate 18j (300 mg) and ethyl acetate (5 mL) were added to a reaction flask in sequence, and a solution of hydrochloric acid in dioxane (4 mol/L, 3.89 mL, 15.58 mmol) was added. The mixture was reacted at room temperature. After the reaction was completed, as confirmed by TLC, the reaction solution was concentrated to give compound 18 (235 mg).

MS(ESI, [M+H]⁺) m/z: 286.10.

¹H NMR (500 MHz, DMSO-d6) δ 11.08 (s, 1H), 7.55 (d, J=8.1 Hz, 1H), 7.10 (d, J=8.2 Hz, 1H), 4.55 (dd, J=11.9, 5.0 Hz, 1H), 4.07 (s, 2H), 3.00 (t, J=5.7 Hz, 2H), 2.81 (t, J=5.7 Hz, 2H), 2.78-2.71 (m, 1H), 2.60 (dt, J=17.3, 4.2 Hz, 1H), 2.46 (dd, J=12.2, 4.5 Hz, 1H), 2.18 (dq, J=13.6, 4.9 Hz, 1H).

### Example 19: Synthesis of Compound 19

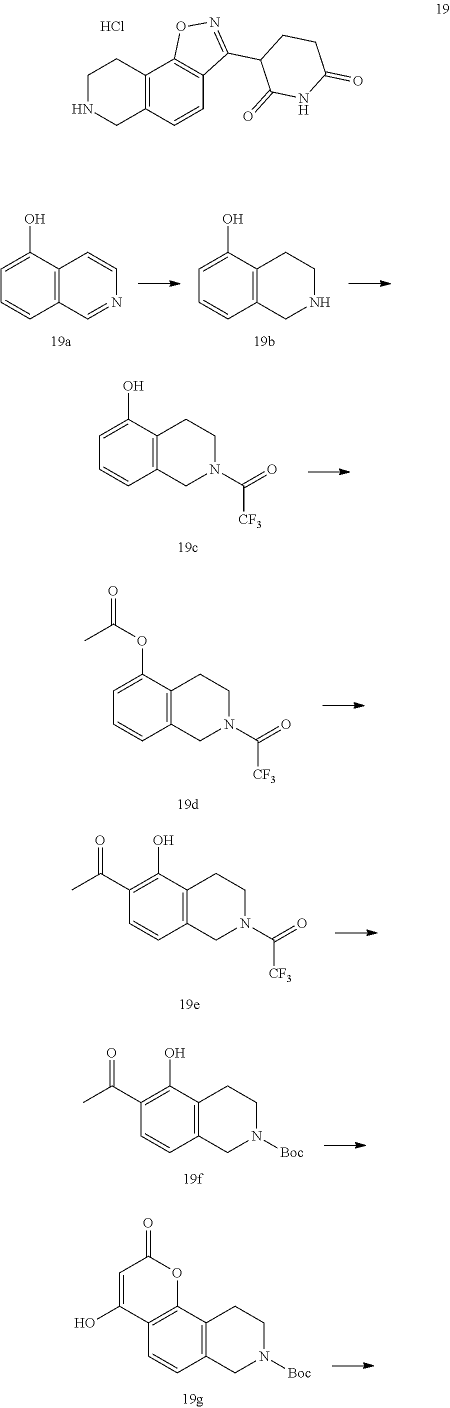

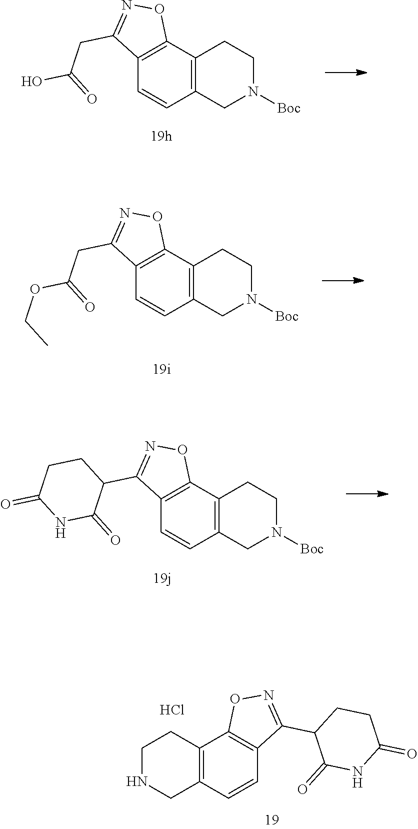 [SMILES: CC(C)(C)OC(=O)N1CCc2c(ccc3c(CC(=O)O)noc23)C1 ; CCOC(=O)Cc1noc2c3c(ccc12)CN(C(=O)OC(C)(C)C)CC3 ; CC(C)(C)OC(=O)N1CCc2c(ccc3c(C4CCC(=O)NC4=O)noc23)C1 ; O=C1CCC(c2noc3c4c(ccc23)CNCC4)C(=O)N1 ; Cl]

### Step 1: Preparation of Intermediate 19b

Intermediate 19a was completely dissolved in methanol (1680 mL) and acetic acid (166 mL) and then added to a high pressure reactor under a hydrogen pressure of 3 Mpa and a temperature of 110° C. After the reaction was completed, as confirmed by TLC, the reaction solution was concentrated by evaporation at reduced pressure to remove the solvent, and a solution of hydrochloric acid in dioxane (4 mol/L, 200 mL, 798 mmol) was added to the residue. The mixture was concentrated by evaporation at reduced pressure to remove the solvent, the residue was slurried with ethyl acetate and filtered, and the filter cake was collected to give the target intermediate 19b (46.96 g).

MS(ESI, [M+H]⁺) m/z: 150.0.

¹H NMR (500 MHz, DMSO-d6) δ 9.81 (s, 1H), 7.03 (t, J=7.7 Hz, 1H), 6.78 (d, J=7.8 Hz, 1H), 6.62 (d, J=7.6 Hz, 1H), 4.15 (s, 2H), 3.35 (s, 2H), 2.79 (s, 2H).

### Step 2: Preparation of Intermediate 19c

Intermediate 19b (40 g) and tetrahydrofuran (400 mL) were added to a reaction flask in sequence, and trifluoroacetic anhydride (61.9 g, 41.0 mL, 295 mmol) was added under an ice bath. The mixture was allowed to return to room temperature and react. After the reaction was completed as confirmed by TLC, the reaction solution was extracted with ethyl acetate and water. The organic phase was separated, washed with 1000 mL of saturated brine, dried over anhydrous sodium sulfate, and filtered, and the filtrate was concentrated by evaporation at reduced pressure to remove the solvent, thus giving the target intermediate 19c (81 g).

MS(ESI, [M+H]⁻) m/z: 244.04.

### Step 3: Preparation of Intermediate 19d

Intermediate 19c (65.3 g), dichloromethane (650 mL), triethylamine (81 g, 111 mL, 799 mmol), and DMAP (0.813 g) were added to a reaction flask in sequence, and acetic anhydride (29.9 g, 27.9 mL, 293 mmol) was added under an ice bath. The mixture was allowed to return to room temperature and react. After the reaction was completed, as confirmed by TLC, the reaction solution was concentrated by evaporation at reduced pressure to remove the solvent, and the residue was extracted with ethyl acetate and water. The organic phase was separated, washed with a saturated ammonium chloride solution and saturated brine, dried over anhydrous sodium sulfate, and filtered, and the filtrate was concentrated by evaporation at reduced pressure to remove the solvent, thus giving the target intermediate 19d (55.6 g).

¹H NMR (500 MHz, Chloroform-d) δ 7.28 (d, J=7.9 Hz, 1H), 7.10-7.02 (m, 1H), 7.02-6.95 (m, 1H), 4.79 (d, J=27.0 Hz, 2H), 3.92-3.78 (m, 2H), 2.82-2.72 (m, 2H), 2.33 (d, J=2.0 Hz, 3H).

### Step 4: Preparation of Intermediate 19e

Intermediate 19d (30.73 g) and aluminum trichloride (21.40 g) were added to a reaction flask in sequence and the mixture was heated to 170° C. and reacted under N₂ atmosphere. After the reaction was completed, as confirmed by TLC, the reaction solution was cooled to room temperature, water was added to quench the reaction, and dichloromethane was added for extraction. The organic phase was separated, and the aqueous phase was extracted twice with dichloromethane. The organic phases were combined, washed with saturated brine, dried over anhydrous sodium sulfate, and filtered, and the filtrate was purified by silica gel column chromatography to give the target intermediate 19e (16.21 g).

MS(ESI, [M+H]⁻) m/z: 286.0.

1H NMR (500 MHz, DMSO-d6) δ 12.74 (d, J=11.3 Hz, 1H), 7.82 (d, J=8.3 Hz, 1H), 6.91 (dd, J=14.9, 8.3 Hz, 1H), 4.80 (d, J=9.9 Hz, 2H), 3.86 (dt, J=8.2, 5.9 Hz, 2H), 2.77 (dt, J=17.7, 6.1 Hz, 2H), 2.64 (s, 3H).

### Step 5: Preparation of Intermediate 19f

Intermediate 19e (15.7 g) and MeOH (160 mL) were added to a reaction flask in sequence, and a solution of sodium hydroxide (6.56 g) in water (160 mL) was added dropwise under an ice bath. The mixture was allowed to return to room temperature and react. After the reaction was completed, as confirmed by TLC, the reaction solution was concentrated by evaporation at reduced pressure to remove methanol, and 1,4-dioxane (160 mL) and di-tert-butyl dicarbonate (13.12 g, 13.96 mL) were added. The mixture was reacted at room temperature. After the reaction was completed, as confirmed by TLC, the reaction solution was extracted with ethyl acetate and water. The organic phase was separated, and the aqueous phase was extracted twice with ethyl acetate. The organic phases were combined, washed with saturated brine, dried over anhydrous sodium sulfate, and filtered, and the filtrate was concentrated by evaporation at reduced pressure to remove the solvent, thus giving the target intermediate 19f (19.83 g).

MS(ESI, [M+H]⁺) m/z: 192.0.

¹H NMR (500 MHz, DMSO-d6) δ 12.76 (s, 1H), 7.76 (d, J=8.3 Hz, 1H), 6.80 (d, J=8.3 Hz, 1H), 4.52 (s, 2H), 3.57 (d, J=1.8 Hz, 2H), 2.64 (d, J=6.1 Hz, 5H), 1.43 (s, 9H).

### Step 6: Preparation of Intermediate 19g

Intermediate 19f (15.92 g, 54.6 mmol), diethyl carbonate (32.3 g, 33.1 mL), and toluene (200 mL) were added to a reaction flask in sequence, 60 wt % sodium hydride (10.93 g, 273 mmol) was added in portions under an ice bath. The mixture was heated to 120° C. and reacted. After the reaction was completed, as confirmed by TLC, the reaction solution was cooled to room temperature and poured into ice water to quench the reaction. The pH was adjusted to 1-2 with a 1 M hydrochloric acid solution, and ethyl acetate was added for extraction. The organic phase was separated, and the aqueous phase was extracted twice with ethyl acetate. The organic phases were combined, washed with saturated brine, dried over anhydrous sodium sulfate, and filtered, and the filtrate was concentrated by evaporation at reduced pressure to remove the solvent. The residue was slurried with petroleum ether and filtered, and the filter cake was collected to give the target intermediate 19g (12 g).

¹H NMR (500 MHz, DMSO-d6) δ 12.46 (s, 1H), 7.64 (d, J=8.1 Hz, 1H), 7.17 (d, J=8.2 Hz, 1H), 5.56 (s, 1H), 4.60 (s, 2H), 3.62 (t, J=6.0 Hz, 2H), 2.83 (t, J=5.8 Hz, 2H), 1.44 (s, 9H).

### Step 7: Preparation of Intermediate 19h

Intermediate 19g (12 g), EtOH (120 mL), and an aqueous hydroxylamine solution (12.49 g, 11.59 mL, 189 mmol) were added to a reaction flask in sequence, and the mixture was heated to 85° C. and reacted. After the reaction was completed, as confirmed by TLC, a saturated sodium bicarbonate solution was added to the reaction solution to adjust the pH to 8, and ethyl acetate was added for extraction. The organic phase was separated, and the aqueous phase was adjusted to pH of 3 with 1 M hydrochloric acid and extracted three times with ethyl acetate. The organic phases were combined, washed with saturated brine, dried over anhydrous sodium sulfate, and filtered, and the filtrate was concentrated by evaporation at reduced pressure to remove the solvent, thus giving the target intermediate 19h (10.44 g).

MS(ESI, [M+H]⁻) m/z: 331.0.

¹H NMR (500 MHz, DMSO-d6) δ 12.85 (s, 1H), 7.63 (d, J=8.1 Hz, 1H), 7.21 (d, J=8.2 Hz, 1H), 4.67 (s, 2H), 4.07 (s, 2H), 3.69 (t, J=5.9 Hz, 2H), 2.98 (t, J=5.9 Hz, 2H), 1.44 (s, 9H).

### Step 8: Preparation of Intermediate 191

Intermediate 19h (10.44 g), potassium carbonate (13.02 g), DMA (110 mL), and iodoethane (5.88 g, 3.05 mL, 37.7 mmol) were added to a reaction flask in sequence, and the mixture was heated to 80° C. and reacted under N₂ atmosphere. After the reaction was completed, as confirmed by TLC, the reaction solution was cooled to room temperature and extracted with ethyl acetate and water. The organic phase was separated, washed with 800 mL of saturated brine, dried over anhydrous sodium sulfate, and filtered, and the filtrate was purified by silica gel column chromatography to give the target intermediate 191 (8.31 g).

MS(ESI, [M+H]⁺) m/z: 361.2.

### Step 9: Preparation of Intermediate 19j

Intermediate 191 (5.5 g), tetrahydrofuran (50 mL), and acrylamide (0.759 g, 10.68 mmol) were added to a reaction flask in sequence, the mixture was cooled to −15° C. under N₂ atmosphere, and then a solution of potassium tert-butoxide in tetrahydrofuran (1 mol/L, 9.92 mL, 9.92 mmol) was added dropwise. After the dropwise addition was completed, the mixture was warmed to 0° C. and reacted. After the reaction was completed, as confirmed by TLC, the system was added to a saturated ammonium chloride solution and extracted with ethyl acetate. The organic phase was separated, dried over anhydrous sodium sulfate, and filtered, and the filtrate was purified by silica gel column chromatography to give the target intermediate 19j (2.81 g).

MS(ESI, [M+H]⁻) m/z: 384.34.

¹H NMR (500 MHz, DMSO-d6) δ 11.09 (s, 1H), 7.64 (d, J=8.2 Hz, 1H), 7.20 (d, J=8.3 Hz, 1H), 4.67 (s, 2H), 4.57 (dd, J=12.0, 5.0 Hz, 1H), 3.69 (t, J=5.9 Hz, 2H), 2.98 (t, J=5.9 Hz, 2H), 2.77 (ddd, J=17.3, 12.1, 5.3 Hz, 1H), 2.61 (dt, J=17.3, 4.1 Hz, 1H), 2.54 (d, J=4.5 Hz, 1H), 2.18 (dtd, J=13.5, 5.2, 3.7 Hz, 1H), 1.44 (s, 9H).

### Step 10: Preparation of Compound 19

Intermediate 19j (850 mg) and ethyl acetate (20 mL) were added to a reaction flask in sequence, and a solution of hydrochloric acid in dioxane (4 mol/L, 11.03 mL, 44.1 mmol) was added. The mixture was reacted at room temperature. After the reaction was completed, as confirmed by TLC, the reaction solution was concentrated to give compound 19 (760 mg).

MS(ESI, [M+H]⁺) m/z: 286.12.

¹H NMR (500 MHz, DMSO-d6) δ 11.08 (s, 1H), 7.54 (d, J=8.1 Hz, 1H), 7.05 (d, J=8.2 Hz, 1H), 4.54 (dd, J=11.8, 5.0 Hz, 1H), 3.97 (s, 2H), 3.03 (t, J=5.8 Hz, 2H), 2.86 (t, J=5.8 Hz, 2H), 2.76 (td, J=12.0, 5.9 Hz, 1H), 2.60 (dt, J=17.3, 4.2 Hz, 1H), 2.46 (dd, J=12.2, 4.4 Hz, 1H), 2.18 (dq, J=13.5, 4.8 Hz, 1H).

### Example 20: Synthesis of Compound 20

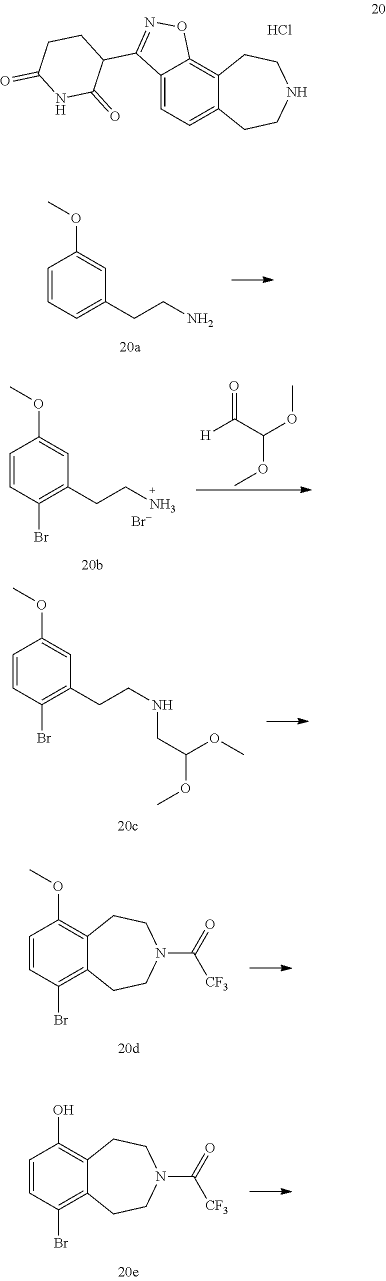

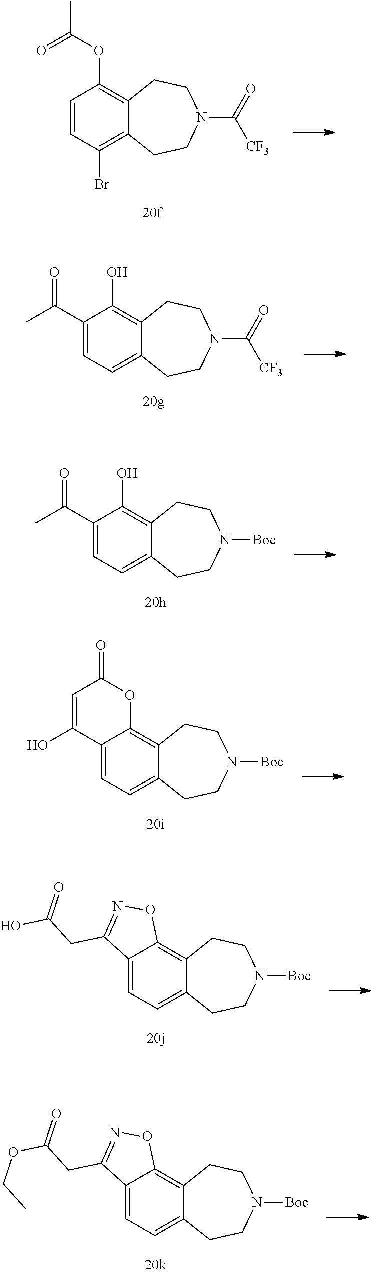 [SMILES: CC(=O)Oc1ccc(Br)c2c1CCN(C(=O)C(F)(F)F)CC2 ; CC(=O)c1ccc2c(c1O)CCN(C(=O)C(F)(F)F)CC2 ; CC(=O)c1ccc2c(c1O)CCN(C(=O)OC(C)(C)C)CC2 ; CC(C)(C)OC(=O)N1CCc2ccc3c(O)cc(=O)oc3c2CC1 ; CC(C)(C)OC(=O)N1CCc2ccc3c(CC(=O)O)noc3c2CC1 ; CCOC(=O)Cc1noc2c3c(ccc12)CCN(C(=O)OC(C)(C)C)CC3]

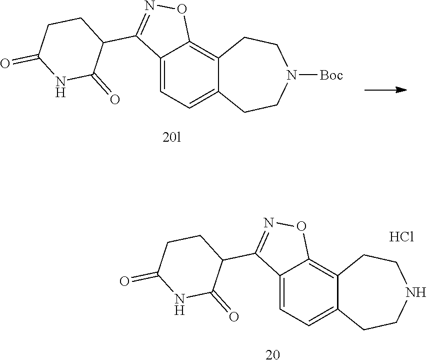 [SMILES: CC(C)(C)OC(=O)N1CCc2ccc3c(C4CCC(=O)NC4=O)noc3c2CC1 ; O=C1CCC(c2noc3c4c(ccc23)CCNCC4)C(=O)N1 ; Cl]

### Step 1: Preparation of Intermediate 20b

Liquid bromine (55.5 g) was added dropwise into a solution of 20a (50 g) in acetic acid (180 mL) at 15° C. After the dropwise addition was completed, the mixture was allowed to react at room temperature. After the reaction was completed, as confirmed by TLC, methyl tert-butyl ether was added dropwise to the reaction solution, and the mixture was stirred and filtered. The filter cake was collected and dried to give intermediate 20b (95 g).

MS(ESI, [M+H]⁺) m/z: 231.9.

### Step 2: Preparation of Intermediate 20c

20b (80 g), glyoxal dimethyl acetal (66.9 g), triethylamine (27.3 g), anhydrous sodium sulfate (80 g), and methanol (600 mL) were added to a reaction flask in sequence, and the mixture was reacted overnight at room temperature. The reaction solution was cooled to −15° C., and sodium borohydride (14.6 g) was added in portions. After the addition was completed, the mixture was allowed to react at room temperature. After the reaction was completed, as confirmed by TLC, the reaction solution was concentrated to remove about half of the methanol, and dichloromethane and water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 20c (60 g).

MS(ESI, [M+H]⁺) m/z: 319.9.

### Step 3: Preparation of Intermediate 20d

20c (47 g) was added dropwise to trifluoroacetic anhydride (148 g) at 0° C. under nitrogen atmosphere. After the dropwise addition was completed, the mixture was allowed to react at room temperature for 1 h. Trifluoroacetic acid (87 g) was added dropwise, and the mixture was warmed to 40° C. and reacted for 1 h. Triethylsilane (68 g) was added dropwise, and the mixture was warmed to 60° C. and reacted. After the reaction was completed, as confirmed by TLC, 400 mL of ethyl acetate and 600 mL of water were added to the reaction solution. The organic phase was separated, washed three times with a saturated sodium chloride solution, dried over anhydrous sodium sulfate, filtered, and concentrated. The concentrate was separated and purified by silica gel column chromatography to give intermediate 20d (20.5 g).

1H NMR (500 MHz, DMSO-d6) δ 7.46 (dd, J=8.9, 1.7 Hz, 1H), 6.87 (d, J=8.9 Hz, 1H), 3.77 (d, J=2.8 Hz, 3H), 3.67 (ddt, J=14.4, 5.7, 3.4 Hz, 4H), 3.22 (ddd, J=11.9, 6.4, 4.6 Hz, 2H), 3.16-3.06 (m, 2H).

### Step 4: Preparation of Intermediate 20e

A solution of boron tribromide in dichloromethane (146 mL, 1 M) was slowly added dropwise to a stirred solution of 20d (20.5 g) in dichloroethane (200 mL) at 0° C. under nitrogen atmosphere. After the dropwise addition was completed, the mixture was allowed to react at room temperature. After the reaction was completed, as confirmed by TLC, the reaction solution was slowly poured into ice water, stirred for 10 min, and filtered, and the filter cake was collected and dried to give 20e (18.5 g).

MS(ESI, [M−H]⁺) m/z: 337.9.

1H NMR (500 MHz, DMSO-d6) δ 9.74 (s, 1H), 7.26 (dd, J=8.7, 1.3 Hz, 1H), 6.68 (dd, J=8.7, 3.1 Hz, 1H), 3.73-3.61 (m, 4H), 3.23-3.13 (m, 2H), 3.12-3.02 (m, 2H).

### Step 5: Preparation of Intermediate 20f

Acetic anhydride (5.65 g) was slowly added dropwise to a stirred solution of 20e (17.0 g) and triethylamine (7.63 g) in dichloroethane (200 mL) at 0° C. under nitrogen atmosphere. After the dropwise addition was completed, the mixture was allowed to react at room temperature. After the reaction was completed, as confirmed by TLC, the reaction solution was slowly poured into water. The organic phase was separated, washed with a saturated sodium chloride solution, dried over anhydrous sodium sulfate, filtered, and concentrated to give 20f (19.8 g).

MS(ESI, [M+H]⁺) m/z: 381.7.

### Step 6: Preparation of Intermediate 20g

20f (19.5 g), aluminum trichloride (18.7 g), and orthodichlorobenzene (80 mL) were added to a reaction flask in sequence, and the mixture was warmed to 150° C. and reacted. After the reaction was completed, as confirmed by TLC, the reaction solution was cooled to room temperature, and 250 mL of 3 N diluted hydrochloric acid was added, followed by extraction with ethyl acetate. The organic phases were combined, washed with saturated brine, dried over anhydrous sodium sulfate, and filtered. The concentrate was separated and purified by silica gel column chromatography to give intermediate 20g (11.2 g).

MS(ESI, [M−H]⁺) m/z: 300.0.

1H NMR (500 MHz, DMSO-d6) δ 12.83 (d, J=4.8 Hz, 1H), 7.78 (dd, J=8.1, 3.5 Hz, 1H), 6.83 (t, J=8.3 Hz, 1H), 3.69 (ddd, J=12.9, 9.6, 5.9 Hz, 4H), 3.11-3.00 (m, 4H), 2.64 (s, 3H).

### Step 7: Preparation of Intermediate 20h

20g (9.5 g), methanol (100 mL), water (20 mL), and sodium hydroxide (1.9 g) were added to a reaction flask in sequence, and the mixture was reacted at room temperature for 1 h. Di-tert-butyl dicarbonate (8.2 g) was added, and the mixture was reacted at room temperature. After the reaction was completed, as confirmed by TLC, ethyl acetate and water were added to the reaction solution. The organic phase was separated, washed with saturated brine, dried over anhydrous sodium sulfate, and filtered. The concentrate was separated and purified by silica gel column chromatography to give intermediate 20h (8.5 g).

¹H NMR (500 MHz, DMSO-d₆) δ 12.80 (s, 1H), 7.74 (d, J=8.1 Hz, 1H), 6.79 (d, J=8.2 Hz, 1H), 3.45 (dt, J=11.6, 5.0 Hz, 4H), 2.92 (q, J=5.0 Hz, 4H), 2.63 (s, 3H), 1.38 (s, 9H).

### Step 8: Preparation of Intermediate 20i

20h (8.5 g), diethyl carbonate (16.4 g), and toluene (100 mL) were added to a reaction flask in sequence, and 60 wt % sodium hydride (5.57 g) was added in portions. The reaction solution was warmed to 115° C. and reacted. After the reaction was completed, as confirmed by TLC, the reaction solution was cooled to room temperature, and ethyl acetate and water were added to the reaction solution. The organic phase was separated, washed with saturated brine, dried over anhydrous sodium sulfate, and filtered. The concentrate was separated and purified by silica gel column chromatography to give intermediate 201 (9.0 g).

MS(ESI, [M−H]⁺) m/z: 330.1.

### Step 9: Preparation of Intermediate 20j

20i (9.0 g), an aqueous hydroxylamine solution (8.7 g), and ethanol (100 mL) were added to a reaction flask in sequence, and the reaction solution was warmed to 80° C. and reacted. After the reaction was completed, as confirmed by TLC, the reaction solution was cooled to room temperature, and ethyl acetate and water were added to the reaction solution. The organic phase was separated, washed with saturated brine, dried over anhydrous sodium sulfate, and filtered. The concentrate was separated and purified by silica gel column chromatography to give intermediate 20j (8.5 g).

MS(ESI, [M−H]⁺) m/z: 345.4.

¹H NMR (500 MHz, DMSO-d₆) δ 7.53 (d, J=8.0 Hz, 1H), 7.12 (d, J=8.0 Hz, 1H), 3.73 (q, J=13.9, 11.5 Hz, 2H), 3.59-3.54 (m, 2H), 3.52-3.47 (m, 2H), 3.13 (t, J=5.2 Hz, 2H), 3.07-2.98 (m, 2H), 1.40 (s, 9H).

### Step 10: Preparation of Intermediate 20k

20j (8.5 g), potassium carbonate (3.3 g), iodoethane (5.1 g), and DMA (70 mL) were added to a reaction flask in sequence, and the reaction solution was warmed to 80° C. and reacted. After the reaction was completed, as confirmed by TLC, the reaction solution was cooled to room temperature, and ethyl acetate and water were added to the reaction solution. The organic phase was separated, washed with saturated brine, dried over anhydrous sodium sulfate, and filtered. The concentrate was separated and purified by silica gel column chromatography to give intermediate 20k (6.5 g).

MS(ESI, [M−H]⁺) m/z: 373.1.

¹H NMR (500 MHz, DMSO-d₆) δ 7.56 (d, J=8.0 Hz, 1H), 7.22 (d, J=8.0 Hz, 1H), 4.13 (dd, J=13.7, 6.6 Hz, 4H), 3.55 (dt, J=28.2, 5.0 Hz, 4H), 3.17 (s, 2H), 3.05 (t, J=5.2 Hz, 2H), 1.38 (dd, J=9.3, 4.4 Hz, 9H), 1.19 (t, J=6.5 Hz, 3H).

### Step 11: Preparation of Intermediate 20l

A solution of sodium tert-butoxide in tetrahydrofuran (14 mL, 1 M) was slowly added dropwise to a stirred solution of 20k (5.6 g) in tetrahydrofuran (70 mL) at −10° C. under nitrogen atmosphere. After the dropwise addition was completed, the mixture was maintained at this temperature and reacted for 30 min. Acrylamide (0.71 g) was weighed out and dissolved in 5 mL of tetrahydrofuran, and the solution was added dropwise to the reaction solution. The mixture was reacted. After the reaction was completed, as confirmed by TLC, the reaction solution was slowly poured into saturated ammonium chloride, and ethyl acetate was added. The organic phase was separated, washed with a saturated sodium chloride solution, dried over anhydrous sodium sulfate, and filtered. The concentrate was separated and purified by silica gel column chromatography to give intermediate 201 (2.5 g).

MS(ESI, [M−H]⁺) m/z: 397.9.

¹H NMR (500 MHz, DMSO-d₆) δ 11.08 (s, 1H), 7.58 (d, J=8.0 Hz, 1H), 7.21 (d, J=8.1 Hz, 1H), 4.55 (dd, J=12.0, 4.9 Hz, 1H), 3.55 (dt, J=31.4, 5.0 Hz, 4H), 3.22-3.00 (m, 4H), 2.77 (ddd, J=17.3, 12.0, 5.3 Hz, 1H), 2.60 (dt, J=17.3, 4.1 Hz, 1H), 2.46 (dd, J=12.2, 4.4 Hz, 1H), 2.20-2.12 (m, 1H), 1.38 (d, J=6.3 Hz, 9H).

### Step 12: Preparation of Compound 20

20l (2.5 g), ethyl acetate (30 mL), and a solution of hydrochloric acid in dioxane (15 mL, 4 M) were added to a reaction flask in sequence, and the mixture was reacted at room temperature. After the reaction was completed, as confirmed by TLC, the reaction solution was filtered, and the filter cake was collected and dried to give compound 20 (18.5 g).

MS(ESI, [M+H]⁺) m/z: 300.2.

1H NMR (500 MHz, DMSO-d6) δ 11.10 (s, 1H), 9.53 (s, 2H), 7.65 (d, J=8.1 Hz, 1H), 7.27 (d, J=8.1 Hz, 1H), 4.59 (dd, J=12.1, 4.9 Hz, 1H), 3.43 (dd, J=7.0, 3.3 Hz, 2H), 3.37-3.19 (m, 6H), 2.78 (ddd, J=17.3, 12.1, 5.3 Hz, 1H), 2.61 (dt, J=17.3, 4.1 Hz, 1H), 2.17 (dtd, J=13.4, 5.2, 3.6 Hz, 1H).

### Example 21: Synthesis of Compound 21

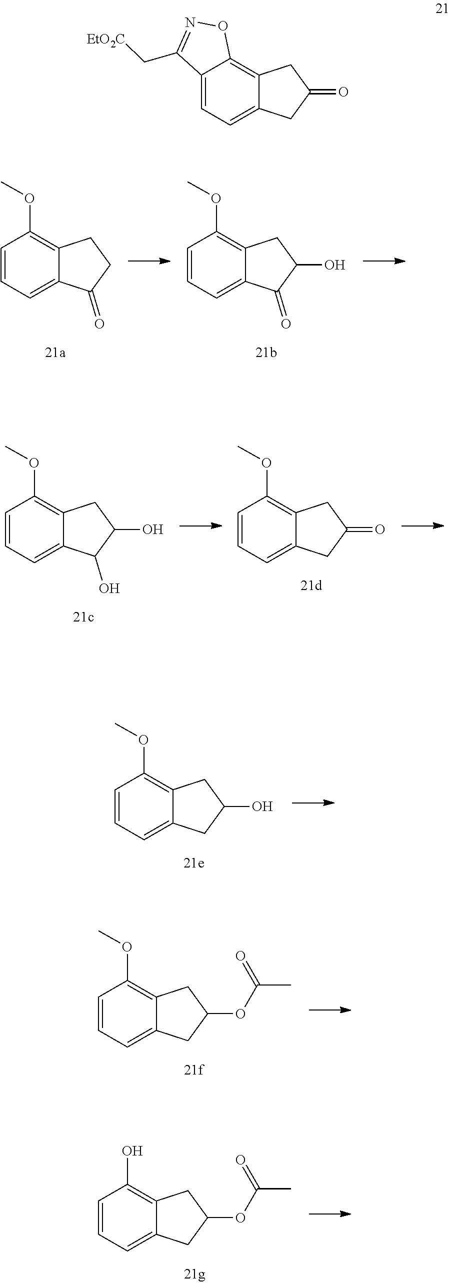

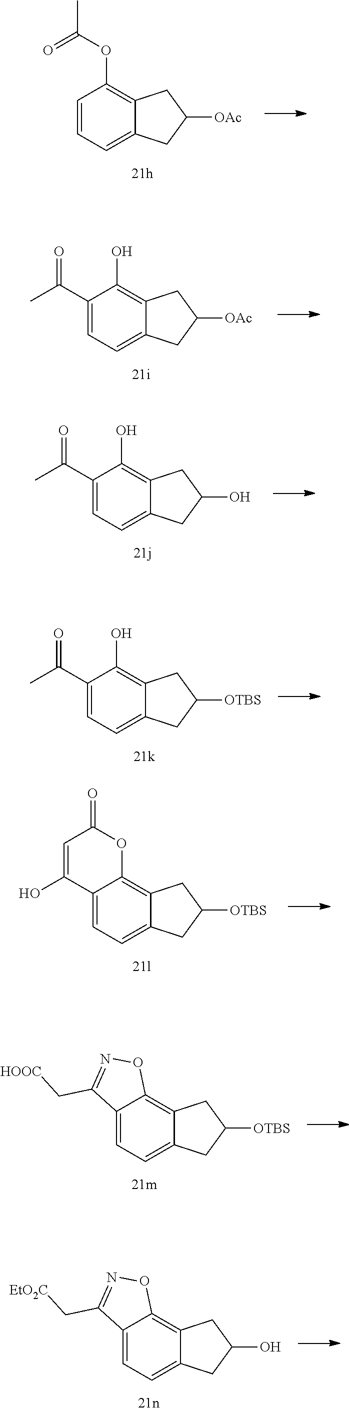

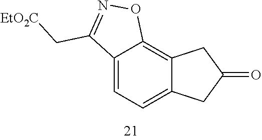 [SMILES: CCOC(=O)Cc1noc2c3c(ccc12)CC(=O)C3]

### Step 1: Preparation of Compound 21b

21a (33.3 g), MeOH (500 mL), iodobenzenediacetic acid (82 g, 246 mmol), and potassium hydroxide (127 g) were added to a reaction flask in sequence under an ice bath, and the mixture was reacted at room temperature. After the reaction was completed, as confirmed by TLC, the reaction solution was concentrated by evaporation at reduced pressure to remove the solvent, and the residue was extracted with ethyl acetate and a sodium bicarbonate solution. The organic phase was separated, dried over anhydrous sodium sulfate, filtered, and concentrated by evaporation at reduced pressure to remove the solvent. The residue was dissolved in THF (500 mL), and hydrochloric acid (6 M, 68.4 mL) was added. The mixture was reacted at room temperature for 0.5 h, and the reaction solution was adjusted to pH of 8 with a saturated sodium bicarbonate solution, extracted with 200 mL of ethyl acetate, dried over anhydrous sodium sulfate, concentrated, and purified by silica gel column chromatography to give 21b (16 g).

MS(ESI, [M+H]⁺) m/z: 178.9.

### Step 2: Preparation of Compound 21c

21b (80 g) and MeOH (1000 mL) were added to a reaction flask in sequence, and after complete dissolution, sodium borohydride (17.83 g, 471 mmol) was added. The mixture was reacted at room temperature. After the reaction was completed, as confirmed by TLC, a saturated ammonium chloride solution was added dropwise to the reaction solution to quench the reaction, and ethyl acetate was added for extraction. The organic phase was dried over anhydrous sodium sulfate, filtered, and concentrated by evaporation at reduced pressure to remove the solvent, thus giving 21c (82 g).

¹H NMR (500 MHz, DMSO-d6) δ 7.17 (t, J=7.8 Hz, 1H), 6.87 (d, J=7.4 Hz, 1H), 6.80 (d, J=8.1 Hz, 1H), 5.34 (d, J=6.3 Hz, 1H), 5.13 (d, J=5.0 Hz, 1H), 4.67 (t, J=5.8 Hz, 1H), 4.06 (ddd, J=12.0, 6.8, 5.1 Hz, 1H), 3.75 (s, 3H), 3.03 (dd, J=15.8, 7.1 Hz, 1H), 2.43 (dd, J=15.8, 6.5 Hz, 1H).

### Step 3: Preparation of Compound 21d

21c (35 g), toluene (300 mL), and p-toluenesulfonic acid (66.9 g) were added to a reaction flask in sequence, and the mixture was heated to 120° C. and reacted. After the reaction was completed, as confirmed by TLC, the reaction solution was cooled to room temperature, and the organic solvent ethyl acetate and water were added to quench the reaction. The organic phase was separated, dried over anhydrous sodium sulfate, filtered, and concentrated by evaporation at reduced pressure to remove the solvent, thus giving 21d (35.5 g).

¹H NMR (500 MHz, DMSO-d6) δ 7.26-7.22 (m, 1H), 6.93-6.87 (m, 2H), 3.79 (s, 3H), 3.53 (s, 2H), 3.37 (s, 2H).

### Step 4: Preparation of Compound 21e

21d (35 g), MeOH (400 mL), and sodium borohydride (5.83 g) were added to a reaction flask in sequence, and the mixture was reacted at room temperature. After the reaction was completed, as confirmed by TLC, a saturated ammonium chloride solution was added dropwise to the reaction solution to quench the reaction, and ethyl acetate was added for extraction. The organic phase was dried over anhydrous sodium sulfate, filtered, and concentrated by evaporation at reduced pressure to remove the solvent, thus giving 21e (18 g).

¹H NMR (500 MHz, DMSO-d6) δ 7.13-7.07 (m, 1H), 6.83-6.78 (m, 1H), 6.74 (d, J=8.1 Hz, 1H), 4.81 (d, J=3.8 Hz, 1H), 4.49 (tq, J=6.5, 3.4 Hz, 1H), 3.75 (s, 3H), 3.04 (dd, J=16.1, 6.1 Hz, 1H), 2.94 (dd, J=16.3, 6.2 Hz, 1H), 2.79-2.61 (m, 2H).

### Step 5: Preparation of Compound 21f

21e (60 g), dichloromethane (500 mL), triethylamine (111 g, 152 mL), and acetic anhydride (41.0 g, 38.2 mL) were added to a reaction flask in sequence, and the mixture was reacted at room temperature. After the reaction was completed, as confirmed by TLC, the reaction solution was washed with a saturated ammonium chloride solution and saturated brine, dried over anhydrous sodium sulfate, filtered, and concentrated under reduced pressure to give 21f (37.2 g).

¹H NMR (500 MHz, DMSO-d6) δ 7.22-7.06 (m, 1H), 6.82 (dd, J=27.3, 8.3 Hz, 2H), 5.41 (s, 1H), 3.78 (d, J=13.2 Hz, 3H), 3.32-3.20 (m, 1H), 3.18-3.09 (m, 1H), 2.85 (dd, J=34.6, 17.1 Hz, 2H), 1.97 (d, J=15.3 Hz, 3H).

### Step 6: Preparation of Compound 21g

Boron trichloride (19.22 g, 164 mL) was slowly added dropwise to a stirred solution of 21f (17 g) in dichloromethane (500 mL) under an ice bath. After the dropwise addition was completed, the mixture was naturally warmed to room temperature and reacted. After the reaction was completed, as confirmed by TLC, 160 mL of 1 M HCl and 200 mL of an aqueous solution were added to the reaction solution to quench the reaction, followed by extraction with dichloromethane. The organic phase was separated, dried over anhydrous sodium sulfate, and concentrated by evaporation at reduced pressure to remove the solvent, thus giving 21g (15 g).

MS(ESI, [M−H]⁻) m/z: 190.9.

### Step 7: Preparation of Compound 21h

21g (16 g), dichloromethane (200 mL), triethylamine (9.50 g, 13.01 mL), and acetic anhydride (5.27 g, 4.91 mL) were added to a reaction flask in sequence, and the mixture was reacted at room temperature. After the reaction was completed, as confirmed by TLC, the reaction solution was washed with a saturated ammonium chloride solution and saturated brine, dried over anhydrous sodium sulfate, filtered, and concentrated under reduced pressure to give 21h (14.8 g).

MS(ESI, [M−H]⁻) m/z: 233.01.

### Step 8: Preparation of Compound 211

21h (11.6 g), dichloromethane (300 mL), and zirconium tetrachloride (46.2 g, 198 mmol) were added to a reaction flask in sequence, and the mixture was reacted at 50° C. After the reaction was completed, as confirmed by TLC, the reaction solution was cooled to room temperature, and a 3 M aqueous hydrochloric acid solution, water, and dichloromethane were added to the residue. The organic phase was separated, dried over anhydrous sodium sulfate, filtered, and concentrated by evaporation at reduced pressure to remove the solvent, thus giving 211 (11.4 g).

¹H NMR (500 MHz, DMSO-d6) δ 12.34 (s, 1H), 7.80 (d, J=8.0 Hz, 1H), 6.91 (d, J=8.0 Hz, 1H), 5.45 (tt, J=6.3, 2.2 Hz, 1H), 3.37-3.33 (m, 1H), 3.19 (dd, J=17.3, 6.3 Hz, 1H), 2.95 (dd, J=17.9, 2.2 Hz, 1H), 2.86 (dd, J=17.3, 2.1 Hz, 1H), 2.63 (s, 3H), 1.97 (s, 3H).

### Step 9: Preparation of Compound 21k

21i (15.4 g), ethanol (200 mL), and an aqueous solution (10.00 mL) of sodium hydroxide (2.63 g) were added to a reaction flask in sequence, and the mixture was reacted at room temperature. After the reaction was completed, as confirmed by TLC, the reaction solution was adjusted to pH of 2-3 with a 2 M aqueous HCl solution, extracted with ethyl acetate and water, dried over anhydrous sodium sulfate, filtered, and concentrated by evaporation at reduced pressure to remove the solvent, thus giving the crude intermediate 21j. Dichloroethane (200 mL), imidazole (17.90 g), and TBSCl (39.6 g) were added, and the mixture was reacted overnight under reflux. The reaction solution was cooled to room temperature, and 100 mL of dichloromethane and 300 mL of water were added for extraction. The organic phase was dried over anhydrous sodium sulfate, filtered, and concentrated by evaporation at reduced pressure to remove the solvent, thus giving 21k (15.4 g).

¹H NMR (500 MHz, DMSO-d6) δ 12.25 (s, 1H), 7.69 (d, J=8.0 Hz, 1H), 6.79 (d, J=8.0 Hz, 1H), 4.64 (dq, J=6.2, 3.2 Hz, 1H), 3.05 (ddd, J=56.3, 16.6, 6.2 Hz, 2H), 2.71 (dd, J=17.0, 3.6 Hz, 1H), 2.61 (dd, J=16.3, 3.5 Hz, 1H), 2.51 (s, 3H), 0.78 (s, 9H), 0.00 (s, 6H).

### Step 10: Preparation of Compound 211

21k (8.4 g) and THF (300 mL) were added to a reaction flask in sequence, and diethyl carbonate (16.19 g, 16.52 mL) was added. The mixture was cooled to about 0° C., and 60 wt % sodium hydride (5.48 g, 137 mmol) was added in portions. The mixture was heated to 85° C. and reacted. After the reaction was completed, as confirmed by TLC, the reaction solution was cooled to room temperature and slowly poured into ice water. The mixture was extracted with ethyl acetate, and the organic phase was discarded. The aqueous phase was adjusted to pH=1-2 with 3 M hydrochloric acid, then extracted with ethyl acetate, dried over anhydrous sodium sulfate, filtered, and concentrated by evaporation at reduced pressure to remove the solvent, thus giving 211 (10 g).

MS(ESI, [M−H]⁻) m/z: 331.2.

### Step 11: Preparation of Intermediate 21m

21l (10 g), an aqueous hydroxylamine solution (9.93 g, 9.93 mL, 150 mmol), and ethanol (100 mL) were added to a reaction flask in sequence, and the mixture was reacted at 85° C. After the reaction was completed, as confirmed by TLC, the reaction solution was cooled to room temperature, ethyl acetate and a saturated aqueous sodium carbonate solution were added to the residue for extraction, and the organic phase was discarded. The aqueous phase was adjusted to pH=2-3 with a 1 M aqueous HCl solution, extracted with ethyl acetate, dried over anhydrous sodium sulfate, filtered, and concentrated by evaporation at reduced pressure to remove the solvent, thus giving 21m (11 g).

MS(ESI, [M−H]⁻) m/z: 346.2.

### Step 12: Preparation of Intermediate 21n

21m (10 g), ethanol (150 mL), and sulfuric acid (14.40 g, 7.83 mL, 144 mmol) were added to a reaction flask in sequence, and the mixture was reacted at 85° C. After the reaction was completed, as confirmed by TLC, the reaction solution was cooled to room temperature and adjusted to pH=7 with dichloromethane and saturated aqueous sodium bicarbonate. The organic phase was separated, dried over anhydrous sodium sulfate, filtered, and concentrated by evaporation at reduced pressure to remove the solvent, thus giving 21n (5.8 g).

MS(ESI, [M+H]+) m/z: 261.97.

¹H NMR (500 MHz, DMSO-d6) δ 7.65 (d, J=8.0 Hz, 1H), 7.33 (d, J=8.0 Hz, 1H), 5.09 (d, J=4.0 Hz, 1H), 4.71 (dt, J=6.4, 3.1 Hz, 1H), 4.26-4.12 (m, 4H), 3.30 (ddd, J=31.0, 16.5, 6.0 Hz, 2H), 2.98 (ddd, J=31.6, 16.5, 3.0 Hz, 2H), 1.22 (t, J=7.1 Hz, 3H).

### Step 13: Preparation of Intermediate 21

Compound 21n (300 mg), dichloromethane (10 mL), and Dess-Martin periodinane (974 mg) were added to a reaction flask in sequence, and the mixture was stirred and reacted at room temperature. After the reaction was completed, as confirmed by TLC, the reaction solution was poured into a saturated sodium sulfite solution to quench the reaction, and ethyl acetate was added for extraction. The organic phase was separated, washed with 200 mL of a saturated sodium bicarbonate solution and 200 mL of saturated brine, dried over anhydrous sodium sulfate, and filtered, and the filtrate was concentrated by evaporation at reduced pressure to remove the solvent, thus giving compound 21 (320 mg).

MS(ESI, [M+H]⁺) m/z: 260.0.

¹H NMR (500 MHz, DMSO-d6) δ 7.74 (d, J=8.1 Hz, 1H), 7.38 (d, J=8.1 Hz, 1H), 4.21 (s, 2H), 4.14 (q, J=7.1 Hz, 2H), 3.79 (s, 2H), 3.72 (s, 2H), 1.19 (t, J=7.1 Hz, 3H).

### Examples 22 and 23: Synthesis of Compounds 22 and 23

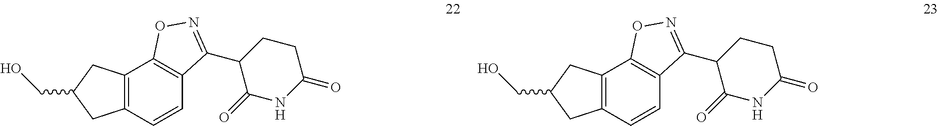 [SMILES: O=C1CCC(c2noc3c4c(ccc23)CC(CO)C4)C(=O)N1 ; O=C1CCC(c2noc3c4c(ccc23)CC(CO)C4)C(=O)N1]

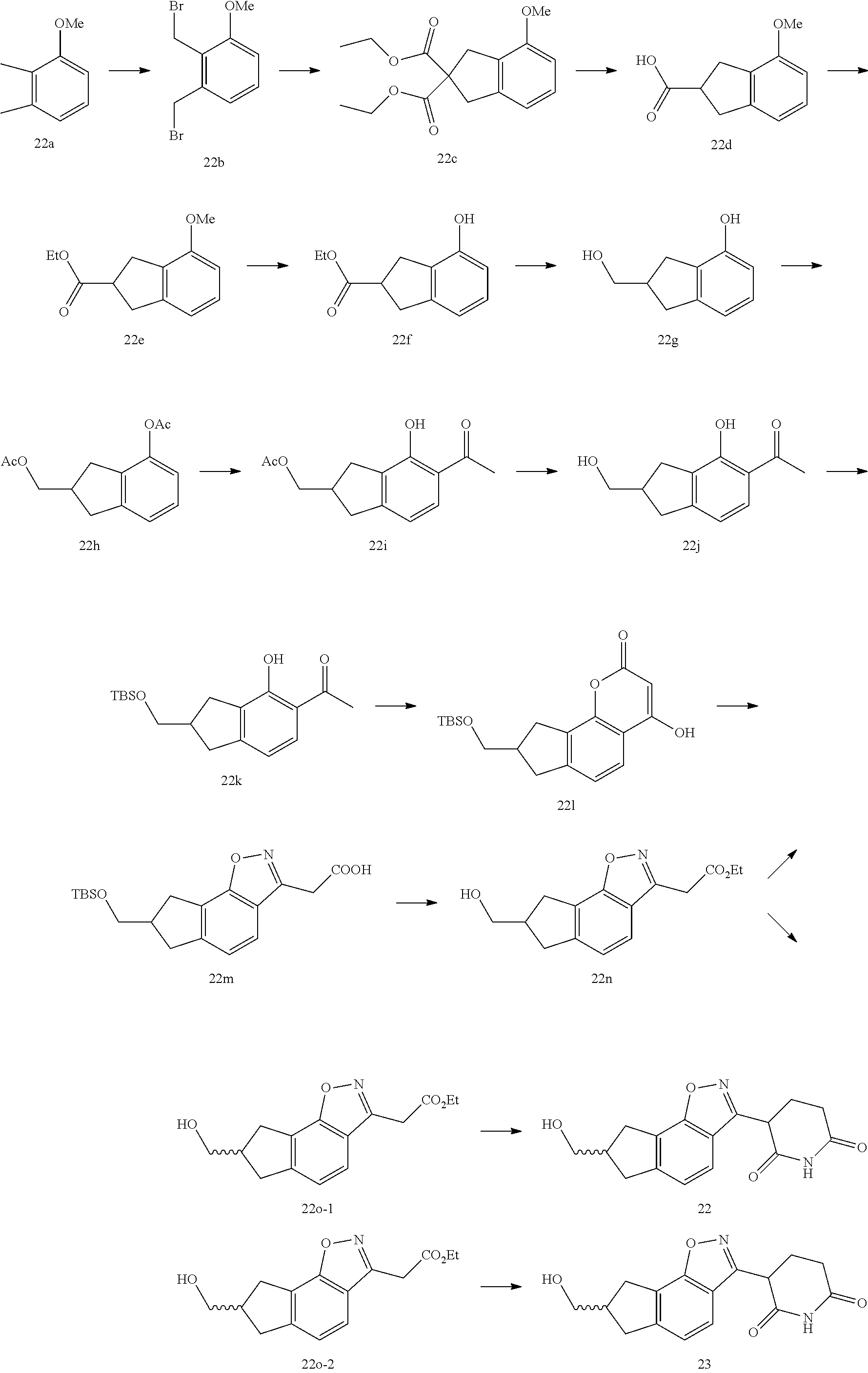

### Step 1: Preparation of Intermediate 22b

CCl₄ (6750 mL), 22a (450 g), 2,2-azobisisobutyronitrile (18.45 g), and N-bromosuccinimide (1194 g) were added to a reaction flask in sequence. The mixture was heated to 80° C. and reacted under reflux. After the reaction was completed, as confirmed by TLC, the reaction solution was filtered, and the filtrate was concentrated by evaporation at reduced pressure to remove the solvent. The residue was slurred with petroleum ether and filtered, and the filter cake was collected to give intermediate 22b (833 g).

¹H NMR (500 MHz, DMSO-d6) δ 7.34 (d, J=8.1 Hz, 1H), 7.06 (ddd, J=17.8, 8.1, 1.1 Hz, 2H), 4.77 (d, J=9.5 Hz, 4H), 3.87 (s, 3H).

### Step 2: Preparation of Intermediate 22c

60 wt % NaH (187 g) and THF (2000 mL) were added to a reaction flask in sequence, and diethyl malonate (300 g, 284 mL) was added under an ice bath. The mixture was stirred at room temperature for 30 mi, and then 22b (606 g) was added. The mixture was stirred and reacted at room temperature. After the reaction was completed, as confirmed by TLC, the reaction solution was slowly added dropwise to a saturated ammonium chloride solution to quench the reaction, and 2000 mL of petroleum ether and 2000 mL of water were added for extraction. The aqueous phase was extracted twice with 1000 mL of petroleum ether, and the organic phases were combined, washed twice with 500 mL of a saturated ammonium chloride solution, washed with saturated brine, dried over anhydrous sodium sulfate, and filtered. The filtrate was concentrated by evaporation at reduced pressure to remove the solvent, and the residue was purified by silica gel column chromatography to give 262 g of intermediate 22c.

MS(ESI, [M−H]+) m/z: 293.2

¹H NMR (500 MHz, DMSO-d6) δ 7.16 (t, J=7.8 Hz, 1H), 6.80 (dd, J=15.3, 7.8 Hz, 2H), 4.14 (q, J=7.1 Hz, 4H), 3.77 (s, 3H), 3.48 (s, 2H), 3.38 (s, 2H), 1.17 (t, J=7.0 Hz, 6H).

### Step 3: Preparation of Intermediate 22d

22c (130 g), DMSO (1000 mL), H₂O (300 mL), and lithium chloride (42.6 g) were added to a reaction flask, and the mixture was stirred at 180° C. After the reaction was completed, as confirmed by TLC, the reaction solution was poured into 1000 mL of ice water to quench the reaction, adjusted to pH=2-3 with 1 M hydrochloric acid, extracted 3 times with 1 L of ethyl acetate, washed with saturated brine, dried over anhydrous sodium sulfate, and filtered, and the filtrate was concentrated by evaporation at reduced pressure to remove the solvent, thus giving 191 g of intermediate 22d.

MS(ESI, [M+H]+) m/z: 193.05

### Step 4: Preparation of Intermediate 22e

22d (85 g), ethanol (1000 mL), and concentrated sulfuric acid (44 g) were added to a reaction flask in sequence, and the mixture was heated to 70° C. and reacted. After the reaction was completed, as confirmed by TLC, the reaction solution was cooled to room temperature and concentrated by evaporation at reduced pressure to remove the solvent. The residue was poured into ice water and neutralized with a saturated aqueous sodium bicarbonate solution. After neutralization, the mixture was extracted 3 times with 1000 mL of petroleum ether. The organic phase was separated, washed with saturated brine, dried over anhydrous sodium sulfate, and filtered, and the filtrate was concentrated by evaporation at reduced pressure to remove the solvent, thus giving 99 g of intermediate 22e.

MS(ESI, [M+H]+) m/z: 221.1

¹H NMR (500 MHz, DMSO-d6) δ 7.13 (t, J=7.8 Hz, 1H), 6.81 (d, J=7.5 Hz, 1H), 6.76 (d, J=8.2 Hz, 1H), 4.09 (q, J=7.1 Hz, 2H), 3.76 (s, 3H), 3.36-3.31 (m, 1H), 3.20-3.10 (m, 2H), 3.10-2.96 (m, 2H), 1.20 (t, J=7.1 Hz, 3H).

### Step 5: Preparation of Intermediate 22f

In a reaction flask, boron tribromide (415 g, 1657 mL) was added dropwise to a stirred solution of 22e (150 g) in dichloromethane (750 mL) under N₂ atmosphere, and the reaction was carried out at a temperature of not higher than 0° C. MeOH (500 mL) was added at 0° C., and the mixture was reacted for 30 min, then gradually allowed to return to room temperature, and stirred for 2 h. After the reaction was completed, the reaction solution was poured into a mixed solvent of 1000 mL of ice water and 1000 mL of dichloromethane, and the mixture was stirred, separated by a separating funnel, washed with saturated brine, dried over anhydrous sodium sulfate, and filtered, and the filtrate was concentrated by evaporation at reduced pressure to remove the solvent, thus giving 122 g of intermediate 22f.

¹H NMR (500 MHz, DMSO-d6) δ 9.24 (s, 1H), 6.95 (t, J=7.7 Hz, 1H), 6.70-6.60 (m, 1H), 6.62-6.52 (m, 1H), 4.10 (q, J=7.1 Hz, 2H), 3.33-3.27 (m, 1H), 3.13-3.01 (m, 3H), 2.96 (dd, J=16.1, 7.1 Hz, 1H), 1.20 (t, J=7.1 Hz, 3H).

### Step 6: Synthesis of Intermediate 22g

22f (130 g) and tetrahydrofuran (2000 mL) were added to a reaction flask in sequence at 0° C. under N₂ atmosphere, and a solution of lithium aluminum hydride in tetrahydrofuran (1 M, 438 mL) was slowly added dropwise. The mixture was reacted under an ice-water bath. After the reaction was completed, as confirmed by TLC, 3 L of water was slowly added dropwise to quench the reaction. The reaction solution was adjusted to pH=1-2 with concentrated hydrochloric acid and extracted 3 times with 2 L of ethyl acetate. The organic phases were combined, washed with saturated brine, dried over anhydrous sodium sulfate, and filtered, and the filtrate was concentrated by evaporation at reduced pressure to remove the solvent, thus giving 129 g of intermediate 22g.

¹H NMR (500 MHz, DMSO-d6) δ 6.88 (t, J=7.7 Hz, 1H), 6.58 (d, J=7.3 Hz, 1H), 6.52 (d, J=7.9 Hz, 1H), 3.38-3.32 (m, 2H), 2.84 (ddd, J=33.5, 16.2, 8.3 Hz, 2H), 2.60 (dq, J=13.9, 8.1, 6.6 Hz, 1H), 2.55-2.49 (m, 3H).

### Step 7: Preparation of Intermediate 22h

22g (120 g), 4-dimethylaminopyridine (7.14 g), dichloromethane (2000 mL), and triethylamine (177 g, 244 mL) were added to a reaction flask in sequence, and acetyl chloride (101 g, 91 mL) was slowly added dropwise at 0° C. After the dropwise addition was completed, the mixture was reacted at room temperature. After the reaction was completed as confirmed by TLC, the reaction solution was poured into a mixed solvent of dichloromethane and water. The organic phase was separated, and the aqueous phase was extracted with dichloromethane. The organic phases were combined, washed with saturated brine, dried over anhydrous sodium sulfate, and filtered, and the filtrate was purified by silica gel column chromatography to give 127 g of intermediate 22h.

MS(ESI, [M+H]+) m/z: 249.3

¹H NMR (500 MHz, DMSO-d6) δ 7.18 (t, J=7.7 Hz, 1H), 7.11 (d, J=7.4 Hz, 1H), 6.88 (d, J=7.9 Hz, 1H), 4.07-3.96 (m, 2H), 3.11-3.00 (m, 1H), 2.87 (dd, J=15.9, 7.8 Hz, 1H), 2.81-2.69 (m, 2H), 2.56-2.50 (m, 1H), 2.27 (s, 3H), 2.02 (s, 3H).

### Step 8: Preparation of Intermediate 22i

22h (91 g), dichloromethane (2000 mL), and zirconium tetrachloride (342 g) were added to a reaction flask in sequence, and the mixture was stirred and reacted overnight at 50° C. under N₂ atmosphere. After the reaction was completed, the reaction solution was cooled to room temperature and then poured into a mixed solvent of 1000 mL of ice water and 1000 mL of dichloromethane. The organic phase was separated, and the aqueous phase was extracted with dichloromethane. The organic phases were combined, dried over anhydrous sodium sulfate, and filtered, and the filtrate was purified by silica gel column chromatography to give 90 g of intermediate 221.

MS(ESI, [M−H]+) m/z: 247.2

¹H NMR (500 MHz, DMSO-d6) δ 12.34 (s, 1H), 7.73 (d, J=8.0 Hz, 1H), 6.83 (d, J=8.0 Hz, 1H), 4.68 (t, J=5.3 Hz, 1H), 3.36 (ddd, J=7.1, 5.3, 2.0 Hz, 2H), 3.03-2.94 (m, 1H), 2.92-2.82 (m, 1H), 2.76-2.68 (m, 1H), 2.61 (s, 3H).

### Step 9: Preparation of Intermediate 22j

22i (95 g) and ethanol (900 mL) were added to a reaction flask in sequence, and a solution of sodium hydroxide (77 g) in H₂O (800 mL) was added dropwise under an ice bath. The mixture was reacted at room temperature under N₂ atmosphere. After the reaction was completed, as confirmed by TLC, the reaction solution was diluted with 2 L of ethyl acetate and 1 L of water, 3 M hydrochloric acid was slowly added to adjust pH=3, and liquid separation was performed. The aqueous phase was extracted 3 times with ethyl acetate, the organic phases were combined, washed with saturated brine, dried over anhydrous sodium sulfate, and filtered, and the filtrate was concentrated by evaporation at reduced pressure to remove the solvent, thus giving 86 g of intermediate 22j.

MS(ESI, [M−H]+) m/z: 205.1

¹H NMR (500 MHz, DMSO-d6) δ 12.34 (s, 1H), 7.73 (d, J=8.0 Hz, 1H), 6.84 (d, J=8.0 Hz, 1H), 4.68 (t, J=5.3 Hz, 1H), 3.36 (ddd, J=7.0, 5.2, 2.0 Hz, 2H), 2.98 (dd, J=17.0, 8.2 Hz, 1H), 2.92-2.82 (m, 1H), 2.72 (dd, J=16.9, 5.6 Hz, 1H), 2.61 (s, 3H), 2.61-2.53 (m, 2H).

### Step 10: Preparation of Intermediate 22k

22j (37 g), 1,2-dichloroethane (700 mL), imidazole (36.6 g), and tert-butyldimethylsilyl chloride (29.7 g) were added to a reaction flask in sequence, and the mixture was reacted at 75° C. After the reaction was completed, as confirmed by TLC, the reaction solution was cooled to room temperature, and dichloromethane and water were added. The organic phase was separated, washed with saturated brine, dried over anhydrous sodium sulfate, and filtered, and the filtrate was concentrated by evaporation at reduced pressure to remove the solvent, thus giving 60 g of intermediate 22k.

¹H NMR (500 MHz, DMSO-d6) δ 12.31 (s, 1H), 7.71 (d, J=8.0 Hz, 1H), 6.82 (d, J=8.0 Hz, 1H), 3.52 (d, J=6.4 Hz, 2H), 2.97 (dd, J=16.9, 8.0 Hz, 1H), 2.85 (dd, J=15.4, 7.5 Hz, 1H), 2.69 (dd, J=16.9, 5.6 Hz, 1H), 2.64-2.59 (m, 1H), 2.58 (s, 3H), 2.55 (d, J=5.6 Hz, 1H), 0.82 (s, 9H), 0.00 (s, 6H).

### Step 11: Preparation of Intermediate 22l

22k (55 g), diethyl carbonate (101 g, 103 mL), and toluene (1000 mL) were added to a reaction flask in sequence. The reaction solution was cooled to 0° C., and 60 wt % sodium hydride (34.3 g, 858 mmol) was added in portions. After the addition was completed, the mixture was slowly heated to 120° C. and reacted. After the reaction was completed as confirmed by TLC, the reaction solution was slowly poured into ice water and extracted with ethyl acetate. The aqueous phase was adjusted to pH=3 with 3 N hydrochloric acid and extracted 3 times with ethyl acetate, and the organic phases were combined, dried over anhydrous sodium sulfate, filtered, and concentrated by evaporation at reduced pressure to remove the solvent, thus giving 53.6 g of intermediate 221.

MS(ESI, [M−H]+) m/z: 345.1.

¹H NMR (500 MHz, DMSO-d6) δ 12.31 (s, 1H), 7.57 (d, J=7.9 Hz, 1H), 7.16 (d, J=7.9 Hz, 1H), 5.49 (s, 1H), 3.56 (d, J=6.4 Hz, 2H), 3.04 (ddd, J=16.0, 13.3, 8.1 Hz, 2H), 2.75 (td, J=14.8, 13.3, 4.4 Hz, 2H), 2.71-2.63 (m, 1H), 0.81 (s, 9H), 0.00 (s, 6H).

### Step 12: Preparation of Intermediate 22m

22l (51 g), hydroxylamine hydrochloride (61.4 g), sodium ethoxide (61.1 g), and ethanol (2000 mL) were added to a reaction flask in sequence, and the mixture was heated to 85° C. and reacted under N₂ atmosphere. After the reaction was completed, as confirmed by TLC, the reaction solution was concentrated by evaporation at reduced pressure to remove the solvent, 2 L of water was added, the pH was adjusted to 8-9 with a saturated sodium carbonate solution, and ethyl acetate was added for extraction. The aqueous phase was collected, adjusted to pH=6-7 with 1 M hydrochloric acid, and extracted with ethyl acetate. The organic phase was collected, washed with saturated brine, dried over anhydrous sodium sulfate, and filtered, and the filtrate was concentrated by evaporation at reduced pressure to remove the solvent, thus giving 47 g of intermediate 22m.

MS(ESI, [M−H]+) m/z: 360.2.

¹H NMR (500 MHz, DMSO-d6) δ 7.55 (d, J=8.0 Hz, 1H), 7.22 (d, J=8.0 Hz, 1H), 3.97 (s, 2H), 3.57 (d, J=6.6 Hz, 2H), 3.16-3.07 (m, 2H), 2.88-2.72 (m, 3H), 0.81 (s, 9H), 0.00 (s, 6H).

### Step 13: Preparation of Intermediate 22n

22m (47 g), ethanol (1500 mL), and concentrated sulfuric acid (65.1 g, 35.4 mL) were added to a reaction flask in sequence, and the mixture was heated to 85° C. under N₂ atmosphere. After the reaction was completed, as confirmed by TLC, the reaction solution was cooled to room temperature and subjected to rotary evaporation to remove the solvent. Dichloromethane was added, and a saturated aqueous sodium bicarbonate solution was added dropwise for neutralization. The mixture was washed with saturated brine, dried over anhydrous sodium sulfate, and filtered, and the filtrate was concentrated by evaporation at reduced pressure to remove the solvent, thus giving 43 g of intermediate 22n.

MS(ESI, [M−H]+) m/z: 276.1

¹H NMR (500 MHz, DMSO-d6) δ 7.58 (d, J=8.0 Hz, 1H), 7.27 (d, J=8.0 Hz, 1H), 4.74 (q, J=4.9 Hz, 1H), 4.16 (s, 2H), 4.12 (t, J=7.1 Hz, 2H), 3.43 (dd, J=6.8, 5.3 Hz, 2H), 3.15 (ddd, J=29.7, 16.4, 8.3 Hz, 2H), 2.93-2.81 (m, 2H), 2.79-2.70 (m, 1H), 1.19 (t, J=7.1 Hz, 3H).

### Step 14: Preparation of Intermediates 22o-1 and 22o-2

Preparative resolution was performed. 43 g of intermediate 22n was dissolved in 430 mL of a solution of dichloromethane in ethanol, the concentration was about 100.0 mg/mL, and the mixture was filtered through a 0.45 μm organic filter membrane and the filtrate was collected. Instrument: YMC high pressure preparative chromatograph; chromatographic column: CHIRALPAK IG (30*250 mm, S-10 μm); mobile phases: A: ethanol, B: n-hexane. The prepeak gave 9.057 g of intermediate 22o-1 and the postpeak gave 8.833 g of intermediate 22o-2.

22o-1: MS(ESI, [M−H]+) m/z: 276.1.

22o-2: MS(ESI, [M−H]+) m/z: 276.1.

### Step 15: Preparation of Compound 22

22o-1 (11.93 g), THF (200 mL), and acrylamide (3.39 g) were added to a reaction flask in sequence under N₂ atmosphere. After the mixture was cooled to 0° C., potassium tert-butoxide (3.89 g, 34.7 mL) was added. The mixture was reacted at 0° C. After the reaction was completed, as confirmed by TLC, the reaction solution was added dropwise to an icy saturated aqueous ammonium chloride solution, and ethyl acetate was added for extraction. The aqueous phase was extracted twice with ethyl acetate, and the organic phases were combined, washed with saturated brine, dried over anhydrous sodium sulfate, and filtered. The filtrate was concentrated by evaporation at reduced pressure to remove the solvent and filtered, and the filter cake was collected to give 6.77 g of compound 22.

¹H NMR (500 MHz, DMSO-d6) δ 11.08 (s, 1H), 7.60 (d, J=8.0 Hz, 1H), 7.25 (d, J=8.1 Hz, 1H), 4.74 (td, J=5.3, 2.2 Hz, 1H), 4.56 (dd, J=11.8, 5.0 Hz, 1H), 3.43 (dd, J=6.8, 5.3 Hz, 2H), 3.22-3.09 (m, 2H), 2.94-2.83 (m, 2H), 2.76 (dq, J=16.9, 6.3 Hz, 2H), 2.60 (dt, J=17.3, 4.2 Hz, 1H), 2.50-2.44 (m, 1H), 2.23-2.13 (m, 1H).

### Step 16: Preparation of Compound of Example 23

22o-2 (12.83 g), THF (200 mL), and acrylamide (3.64 g) were added to a reaction flask in sequence. After the mixture was cooled to 0° C., potassium tert-butoxide (4.18 g, 37.3 mL) was added. The mixture was reacted at 0° C. under N₂ atmosphere. After the reaction was completed, as confirmed by TLC, the reaction solution was added dropwise to an icy saturated aqueous ammonium chloride solution, and ethyl acetate was added for extraction. The aqueous phase was extracted twice with ethyl acetate, and the organic phases were combined, washed with saturated brine, dried over anhydrous sodium sulfate, and filtered. The filtrate was concentrated by evaporation at reduced pressure to remove the solvent and filtered, and the filter cake was collected to give 7.454 g of the compound of Example 23.

¹H NMR (500 MHz, DMSO-d6) δ 11.08 (s, 1H), 7.60 (d, J=8.2 Hz, 1H), 7.25 (d, J=8.1 Hz, 1H), 4.74 (td, J=5.3, 2.2 Hz, 1H), 4.56 (dd, J=11.8, 5.0 Hz, 1H), 3.43 (dd, J=6.8, 5.2 Hz, 2H), 3.21-3.09 (m, 2H), 2.94-2.83 (m, 2H), 2.80-2.71 (m, 2H), 2.60 (dt, J=17.3, 4.2 Hz, 1H), 2.46 (dd, J=12.1, 4.5 Hz, 1H), 2.23-2.15 (m, 1H).

### Example 24: Synthesis of Compound 24

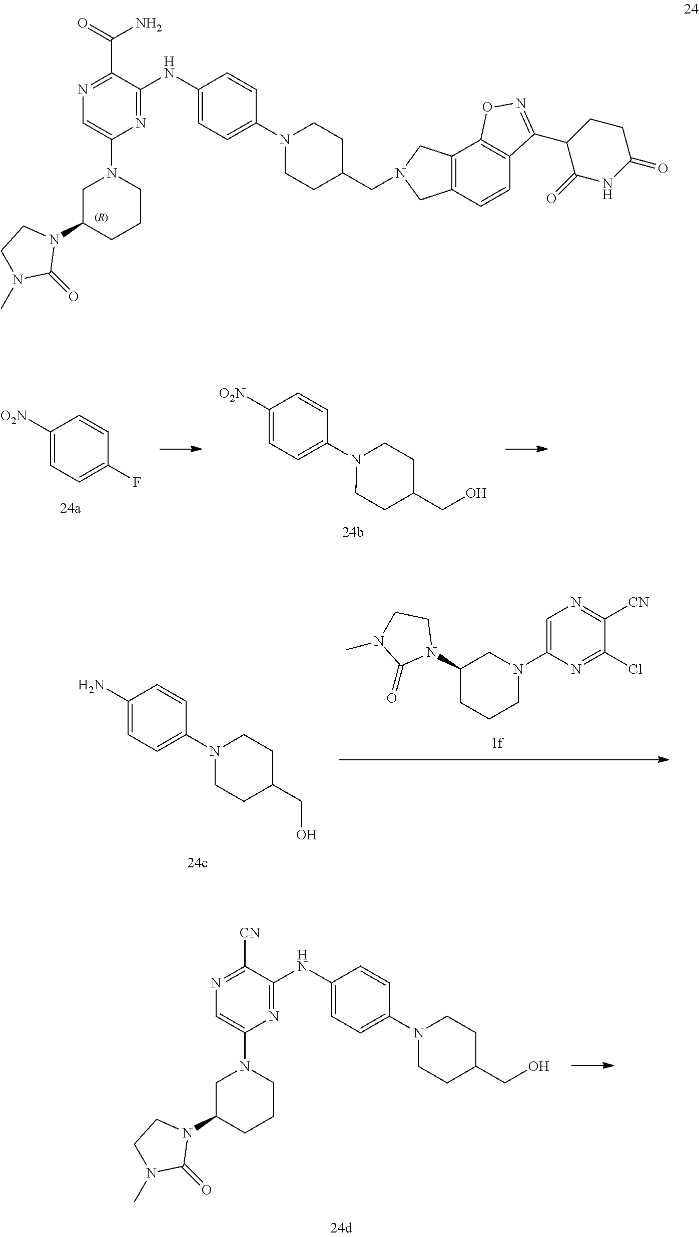

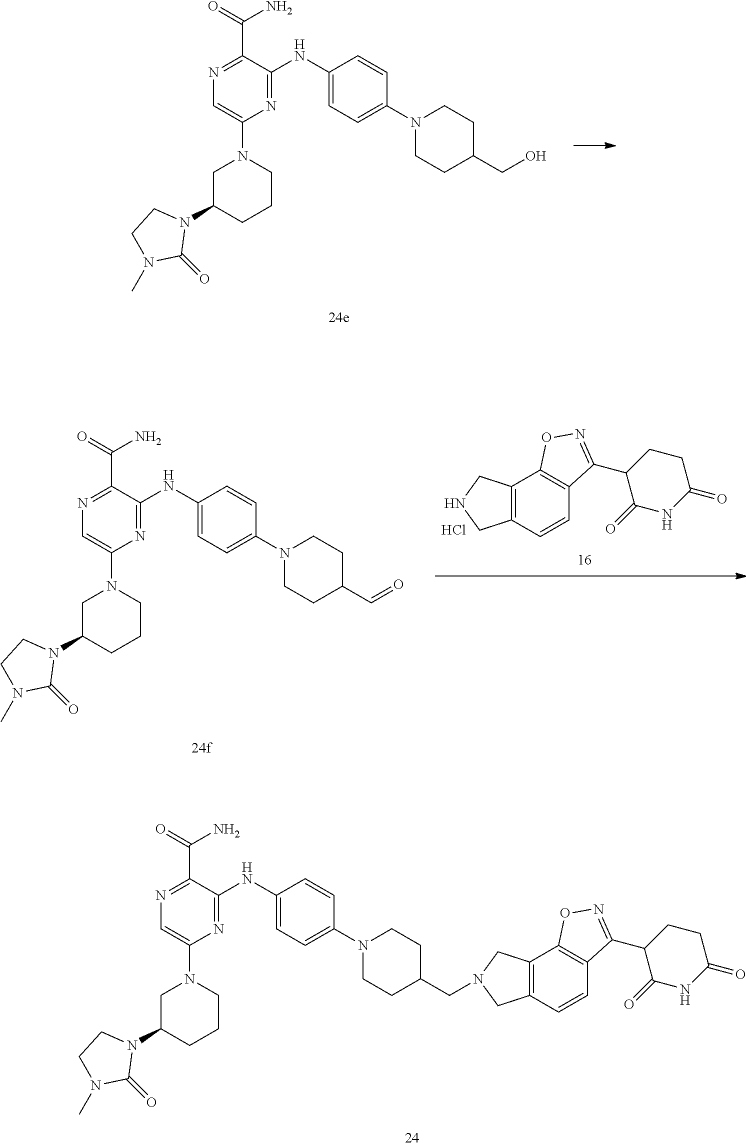 [SMILES: CN1CCN([C@@H]2CCCN(c3cnc(C(N)=O)c(Nc4ccc(N5CCC(CO)CC5)cc4)n3)C2)C1=O ; CN1CCN([C@@H]2CCCN(c3cnc(C(N)=O)c(Nc4ccc(N5CCC(C=O)CC5)cc4)n3)C2)C1=O ; Cl ; O=C1CCC(c2noc3c4c(ccc23)CNC4)C(=O)N1 ; CN1CCN([C@@H]2CCCN(c3cnc(C(N)=O)c(Nc4ccc(N5CCC(CN6Cc7ccc8c(C9CCC(=O)NC9=O)noc8c7C6)CC5)cc4)n3)C2)C1=O]

### Step 1: Preparation of Intermediate 24b

24a (7.00 g), 4-piperidinemethanol (6.86 g), N,N-diisopropylethylamine (9.62 g), and dimethyl sulfoxide (70 mL) were added to a reaction flask in sequence, and the mixture was heated to 100° C. and reacted. After the reaction was completed, as confirmed by TLC, the reaction solution was cooled to room temperature, and water was added to the reaction solution. The mixture was stirred for 10 min and filtered, and the filter cake was collected and dried to give intermediate 24b (13.7 g).

MS(ESI, [M+H]⁺) m/z: 237.1.

### Step 2: Preparation of Intermediate 24c

Intermediate 24b (13.5 g), 10% palladium on carbon (2.70 g), and methanol (200 mL) were added to a reaction flask in sequence, and the mixture was purged three times with hydrogen and reacted overnight. The palladium on carbon was removed by filtration under vacuum, and the residue was concentrated to give intermediate 24c (9.0 g).

MS(ESI, [M+H]⁺) m/z: 207.3.

1H NMR (500 MHz, DMSO-d6) δ 6.68 (d, J=8.2 Hz, 2H), 6.47 (d, J=8.2 Hz, 2H), 4.59 (s, 2H), 4.45 (t, J=5.3 Hz, 1H), 3.34 (s, 2H), 3.28 (dd, J=6.5, 3.1 Hz, 2H), 2.44 (t, J=11.7 Hz, 2H), 1.71 (d, J=12.5 Hz, 2H), 1.40 (ddt, J=11.5, 8.6, 4.1 Hz, 1H), 1.23 (qd, J=12.2, 4.0 Hz, 2H).

### Step 3: Preparation of Intermediate 24d

Intermediate 1f (6.0 g), 24c (3.86 g), BINAP (1.16 g), cesium carbonate (18.28 g), palladium acetate (0.42 g), and 1,4-dioxane (80 mL) were added to a reaction flask in sequence, and the mixture was purged three times with nitrogen, heated to 100° C., and reacted. After the reaction was completed, as confirmed by TLC, the reaction solution was cooled to room temperature, and ethyl acetate and water were added to the residue. The organic phase was separated, washed with a saturated sodium chloride solution, dried over anhydrous sodium sulfate, filtered, and concentrated. The concentrate was separated and purified by silica gel column chromatography to give intermediate 24d (7.1 g).

MS(ESI, [M+H]⁺) m/z: 491.5.

¹H NMR (500 MHz, DMSO-d₆) δ 8.79 (s, 1H), 7.75 (s, 1H), 7.42-7.31 (m, 2H), 6.92-6.79 (m, 2H), 4.47 (t, J 5.3 Hz, 1H), 4.39-4.17 (m, 2H), 3.66-3.52 (m, 3H), 3.31-3.21 (m, 5H), 2.99-2.86 (m, 2H), 2.70 (s, 3H), 2.61-2.53 (m, 2H), 1.81-1.69 (m, 5H), 1.57-1.43 (m, 2H), 1.29-1.16 (m, 3H).

### Step 4: Preparation of Intermediate 24e

Intermediate 24d (6.9 g), cesium carbonate (4.58 g), water (20 mL), DMSO (20 mL), and methanol (70 mL) were added to a reaction flask in sequence. The reaction solution was cooled to 0° C., and 30 wt % hydrogen peroxide (4.78 g) was added dropwise. After the dropwise addition was completed, the mixture was allowed to react at room temperature. After the reaction was completed as confirmed by TLC, water and a saturated aqueous sodium sulfite solution were added to the reaction solution. The mixture was stirred for 10 min and filtered, and the filter cake was collected and dried to give intermediate 24e (6.0 g).

MS(ESI, [M+H]⁺) m/z: 509.5.

¹H NMR (500 MHz, DMSO-d₆) δ 10.97 (s, 1H), 7.70 (d, J=2.8 Hz, 1H), 7.60 (s, 1H), 7.44-7.36 (m, 2H), 7.27 (d, J=2.8 Hz, 1H), 6.91-6.83 (m, 2H), 4.47 (t, J=5.3 Hz, 1H), 4.31 (dd, J=49.3, 12.9 Hz, 2H), 3.66-3.54 (m, 3H), 3.32-3.21 (m, 6H), 3.03-2.88 (m, 2H), 2.70 (s, 3H), 2.57 (td, J=12.1, 2.6 Hz, 2H), 1.84-1.72 (m, 5H), 1.59-1.44 (m, 2H), 1.25 (tt, J=12.1, 2.7 Hz, 2H).

### Step 5: Preparation of Intermediate 24f

Intermediate 24e (2.0 g), dichloromethane (30 mL), and N,N-diisopropylethylamine (1.36 g) were added to a reaction flask in sequence, and the mixture was cooled to 0° C. Sulfur trioxide pyridine (1.88 g) was dissolved in DMSO (6 mL), and the solution was added dropwise to the reaction solution. After the dropwise addition was completed, the mixture was allowed to react at room temperature. After the reaction was completed, as confirmed by TLC, ethyl acetate and water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 24f (1.1 g).

MS(ESI, [M+H]⁺) m/z: 507.4.

### Step 6: Preparation of Compound 24

Intermediate 24f (165 mg), 16 (100 mg), sodium acetate (26.7 mg), and dichloroethane/isopropanol (5:1, 20 mL) were added to a reaction flask in sequence. After the mixture was stirred at room temperature for 30 min, sodium cyanoborohydride (40.8 mg) was added, and the mixture was reacted at room temperature. After the reaction was completed as confirmed by TLC, dichloromethane and water were added to the reaction solution. The organic phase was separated, washed with a saturated sodium chloride solution, dried over anhydrous sodium sulfate, filtered, and concentrated. The concentrate was separated and purified by silica gel column chromatography to give compound 24 (100 mg).

MS(ESI, [M+H]⁺) m/z: 761.6.

1H NMR (500 MHz, DMSO-d6) δ 11.10 (s, 1H), 10.98 (s, 1H), 7.72 (d, J=7.9 Hz, 2H), 7.60 (s, 1H), 7.47-7.37 (m, 2H), 7.35-7.21 (m, 2H), 6.93-6.85 (m, 2H), 4.60 (dd, J=11.9, 5.0 Hz, 1H), 4.32 (dd, J=50.6, 12.8 Hz, 2H), 4.15 (s, 2H), 4.04 (t, J=2.4 Hz, 2H), 3.69-3.55 (m, 3H), 3.35 (dd, J=8.6, 6.2 Hz, 1H), 3.32-3.19 (m, 3H), 2.96 (dt, J=28.7, 11.7 Hz, 2H), 2.77 (ddd, J=17.2, 12.0, 5.3 Hz, 1H), 2.70 (s, 3H), 2.68-2.54 (m, 5H), 2.53 (s, 1H), 2.20 (dq, J=13.5, 4.3 Hz, 1H), 1.96-1.64 (m, 6H), 1.61-1.48 (m, 1H), 1.36-1.27 (m, 2H).

### Example 25: Synthesis of Compound 25

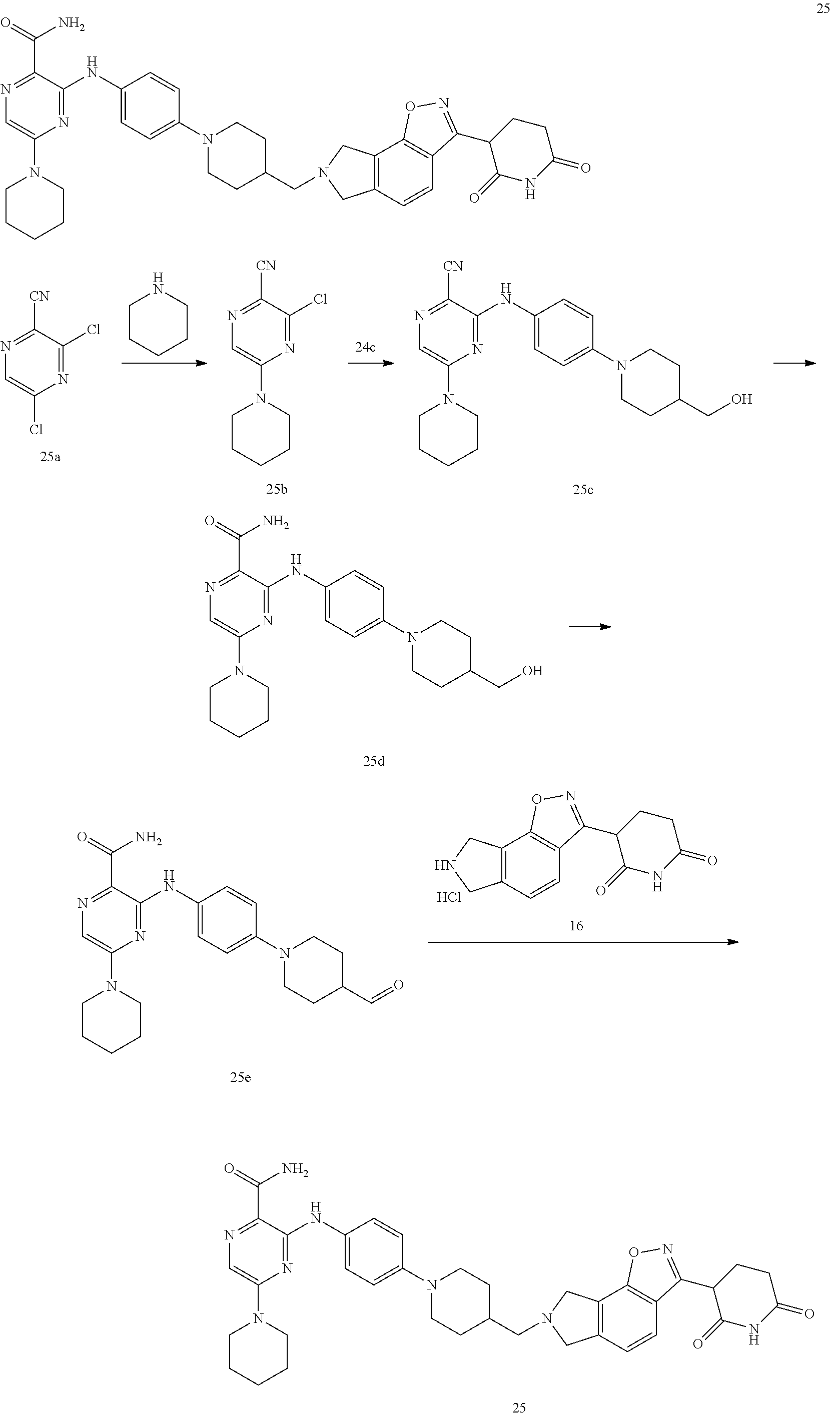

### Step 1: Preparation of Intermediate 25b

25a (5.0 g), tetrahydropyridine (3.49 g), DMF (40 mL), and N,N-diisopropylethylamine (18.57 g, 25.10 mL) were added to a reaction flask in sequence, and the mixture was reacted at room temperature. After the reaction was completed, as confirmed by TLC, water was added to quench the reaction, and ethyl acetate was added for extraction. The organic phase was dried over anhydrous sodium sulfate, filtered, and concentrated by evaporation at reduced pressure to remove the solvent, thus giving 25b (6.6 g).

MS(ESI, [M+H]+) m/z: 223.23.

### Step 2: Preparation of Intermediate 25c

25b (2.00 g), 24c (6.86 g), BINAP (0.51 g), cesium carbonate (8.12 g), palladium acetate (0.18 g), and 1,4-dioxane (50 mL) were added to a reaction flask in sequence, and the mixture was purged three times with nitrogen, heated to 100° C., and reacted. After the reaction was completed, as confirmed by TLC, the reaction solution was cooled to room temperature, and ethyl acetate and water were added to the residue. The organic phase was separated, washed with a saturated sodium chloride solution, dried over anhydrous sodium sulfate, filtered, and concentrated. The concentrate was separated and purified by silica gel column chromatography to give intermediate 25c (2.3 g).

MS(ESI, [M+H]⁺) m/z: 393.4.

### Step 3: Preparation of Intermediate 25d

Intermediate 25c (2.1 g), cesium carbonate (1.74 g), water (10 mL), DMSO (15 mL), and methanol (30 mL) were added to a reaction flask in sequence. The reaction solution was cooled to 0° C., and 30 wt % hydrogen peroxide (1.82 g) was added dropwise. After the dropwise addition was completed, the mixture was allowed to react at room temperature. After the reaction was completed, as confirmed by TLC, water and a saturated aqueous sodium sulfite solution were added to the reaction solution. The mixture was stirred for 10 min and filtered, and the filter cake was collected and dried to give intermediate 25d (2.1 g).

MS(ESI, [M+H]⁺) m/z: 411.3.

### Step 4: Preparation of Intermediate 25e

Intermediate 25d (0.75 g), dichloromethane (20 mL), and N,N-diisopropylethylamine (0.63 g) were added to a reaction flask in sequence, and the mixture was cooled to 0° C. Sulfur trioxide pyridine (0.87 g) was dissolved in DMSO (2 mL), and the solution was added dropwise to the reaction solution. After the dropwise addition was completed, the mixture was allowed to react at room temperature. After the reaction was completed, as confirmed by TLC, ethyl acetate and water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 25e (0.45 g).

MS(ESI, [M+H]⁺) m/z: 409.3.

### Step 5: Preparation of Compound 25

Intermediate 25e (80 mg), 16 (50 mg), sodium acetate (13.3 mg), and dichloroethane/isopropanol (5:1, 20 mL) were added to a reaction flask in sequence. After the mixture was stirred at room temperature for 30 min, sodium cyanoborohydride (20.4 mg) was added, and the mixture was reacted at room temperature. After the reaction was completed, as confirmed by TLC, dichloromethane and water were added to the reaction solution. The organic phase was separated, washed with a saturated sodium chloride solution, dried over anhydrous sodium sulfate, filtered, and concentrated. The concentrate was separated and purified by silica gel column chromatography to give compound 25 (25 mg).

MS(ESI, [M+H]⁺) m/z: 644.4.

1H NMR (500 MHz, DMSO-d6) δ 11.10 (s, 1H), 11.06 (s, 1H), 7.75-7.66 (m, 2H), 7.60 (s, 1H), 7.43 (d, J=8.6 Hz, 2H), 7.31 (d, J=8.1 Hz, 1H), 7.26 (d, J=2.9 Hz, 1H), 6.92 (d, J=8.6 Hz, 2H), 4.60 (dd, J=11.9, 5.0 Hz, 1H), 4.16 (s, 2H), 4.05 (s, 2H), 3.73-3.55 (m, 6H), 2.77 (ddd, J=17.3, 12.0, 5.4 Hz, 1H), 2.63 (ddd, J=24.4, 12.5, 5.4 Hz, 5H), 2.55 (d, J=4.9 Hz, 1H), 2.21 (dt, J=13.3, 4.6 Hz, 1H), 1.93-1.85 (m, 2H), 1.74-1.62 (m, 3H), 1.57 (p, J=5.5, 5.1 Hz, 4H), 1.31 (td, J=11.7, 3.3 Hz, 2H).

### Example 26: Synthesis of Compound 26

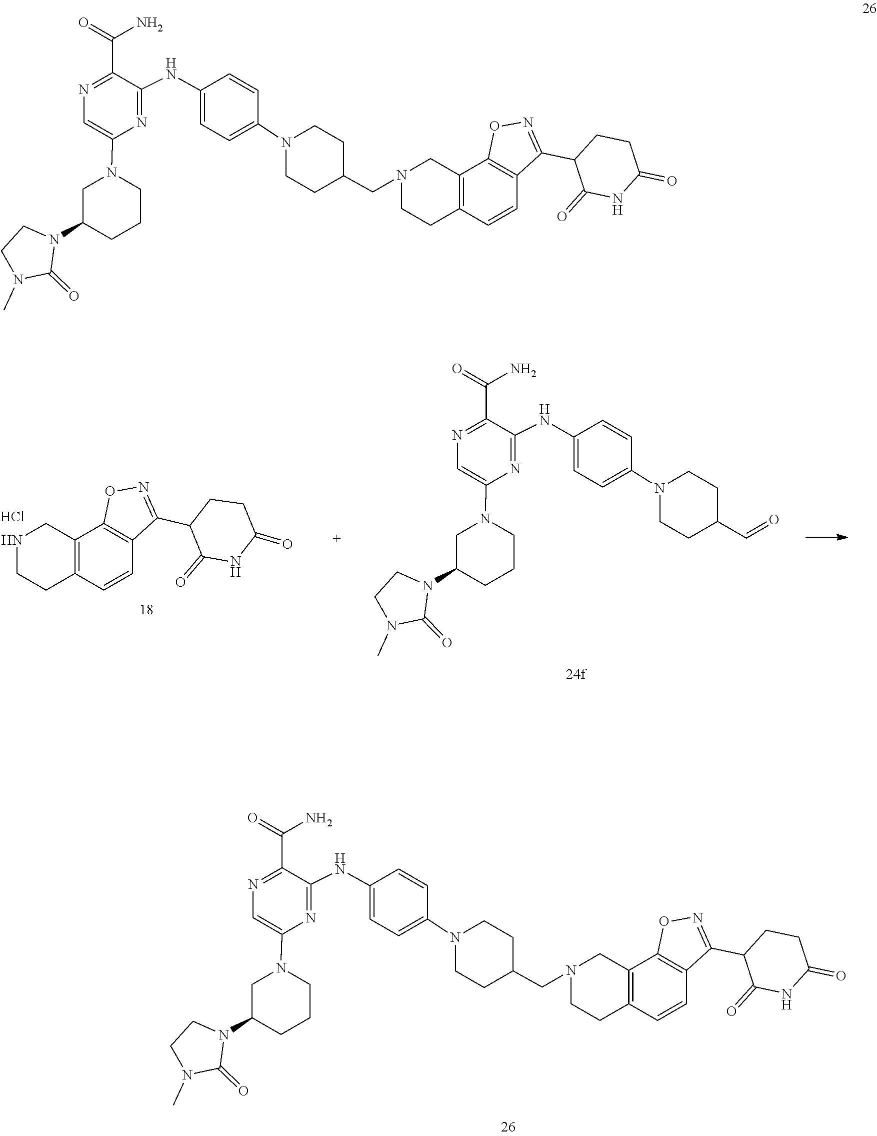 [SMILES: CN1CCN([C@@H]2CCCN(c3cnc(C(N)=O)c(Nc4ccc(N5CCC(CN6CCc7ccc8c(C9CCC(=O)NC9=O)noc8c7C6)CC5)cc4)n3)C2)C1=O ; Cl ; O=C1CCC(c2noc3c4c(ccc23)CCNC4)C(=O)N1 ; CN1CCN([C@@H]2CCCN(c3cnc(C(N)=O)c(Nc4ccc(N5CCC(C=O)CC5)cc4)n3)C2)C1=O ; CN1CCN([C@@H]2CCCN(c3cnc(C(N)=O)c(Nc4ccc(N5CCC(CN6CCc7ccc8c(C9CCC(=O)NC9=O)noc8c7C6)CC5)cc4)n3)C2)C1=O]

Compound 18 (107 mg), intermediate 24f (60 mg), acetic acid (6.31 mg, 6.02 μL), 1,2-dichloroethane (5 mL), and isopropanol (2 mL) were added to a reaction flask in sequence. After the mixture was stirred and reacted at room temperature for 0.5 h, sodium cyanoborohydride (39.6 mg) was added, and the mixture was stirred and reacted at room temperature. After the reaction was completed, as confirmed by TLC, a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 26 (124 mg).

MS(ESI, [M+H]⁺) m/z: 776.58.

¹H NMR (500 MHz, DMSO-d6) δ 11.09 (s, 1H), 10.97 (s, 1H), 7.71 (d, J=2.9 Hz, 1H), 7.63-7.56 (m, 2H), 7.41 (d, J=8.6 Hz, 2H), 7.28 (d, J=2.9 Hz, 1H), 7.15 (d, J=8.2 Hz, 1H), 6.87 (d, J=8.6 Hz, 2H), 4.56 (dd, J=11.9, 5.0 Hz, 1H), 4.36 (d, J=12.6 Hz, 1H), 4.26 (d, J=13.2 Hz, 1H), 3.81 (s, 2H), 3.64-3.56 (m, 3H), 3.27 (ddd, J=29.3, 13.9, 6.4 Hz, 4H), 3.02-2.89 (m, 4H), 2.81-2.72 (m, 3H), 2.70 (s, 3H), 2.62 (ddd, J=21.5, 10.8, 7.6 Hz, 3H), 2.53 (d, J=4.9 Hz, 1H), 2.45 (d, J=6.5 Hz, 2H), 2.18 (dq, J=13.6, 4.8 Hz, 1H), 1.90-1.69 (m, 6H), 1.59-1.49 (m, 1H), 1.27 (d, J=12.3 Hz, 2H).

### Example 27: Synthesis of Compound 27

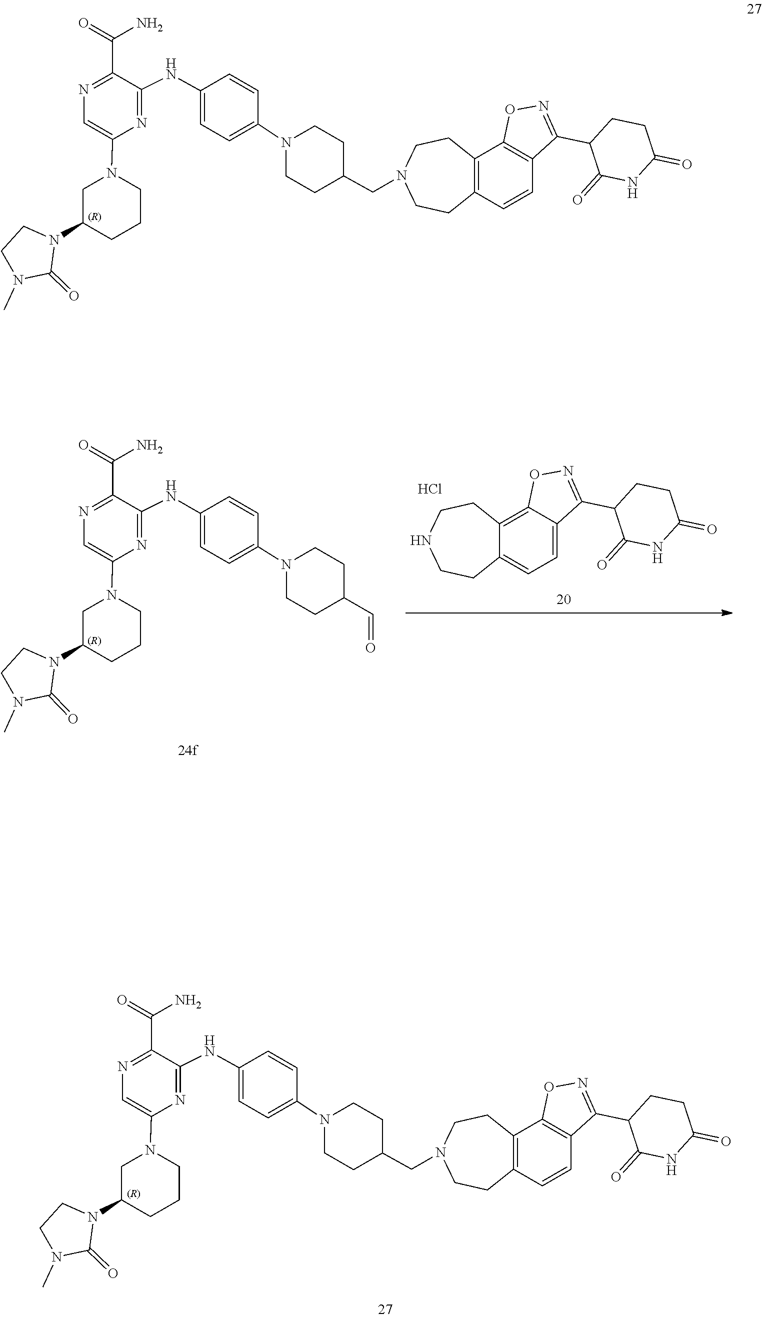

### Step 1: Preparation of Compound 27

Intermediate 24f (113 mg), 20 (75 mg), sodium acetate (18.3 mg), and dichloroethane/isopropanol (5:1, 20 mL) were added to a reaction flask in sequence. After the mixture was stirred at room temperature for 30 min, sodium cyanoborohydride (28.1 mg) was added, and the mixture was reacted at room temperature for 1 h. 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 27 (90 mg).

MS(ESI, [M+H]⁺) m/z: 790.6.

¹H NMR (500 MHz, DMSO-d6) δ 11.09 (s, 1H), 10.98 (s, 1H), 7.71 (d, J=3.0 Hz, 1H), 7.60 (s, 1H), 7.54 (d, J=8.0 Hz, 1H), 7.45-7.39 (m, 2H), 7.28 (d, J=2.9 Hz, 1H), 7.18 (d, J=8.2 Hz, 1H), 6.88 (d, J=9.0 Hz, 2H), 4.55 (dd, J=11.9, 5.0 Hz, 1H), 4.32 (dd, J=47.1, 12.9 Hz, 2H), 3.61 (dd, J=9.7, 5.8 Hz, 3H), 3.35 (dd, J=7.4, 2.1 Hz, 1H), 3.31-3.22 (m, 3H), 3.16 (dd, J=6.9, 2.9 Hz, 2H), 3.05 (dd, J=6.5, 3.3 Hz, 2H), 2.97 (dt, J=32.0, 12.3 Hz, 2H), 2.71 (s, 4H), 2.70-2.55 (m, 7H), 2.47 (dd, J=12.2, 4.4 Hz, 1H), 2.36 (d, J=7.1 Hz, 2H), 2.18 (dt, J=9.1, 3.1 Hz, 1H), 1.88-1.63 (m, 6H), 1.61-1.50 (m, 1H), 1.32-1.24 (m, 2H).

### Example 28: Synthesis of Compound 28

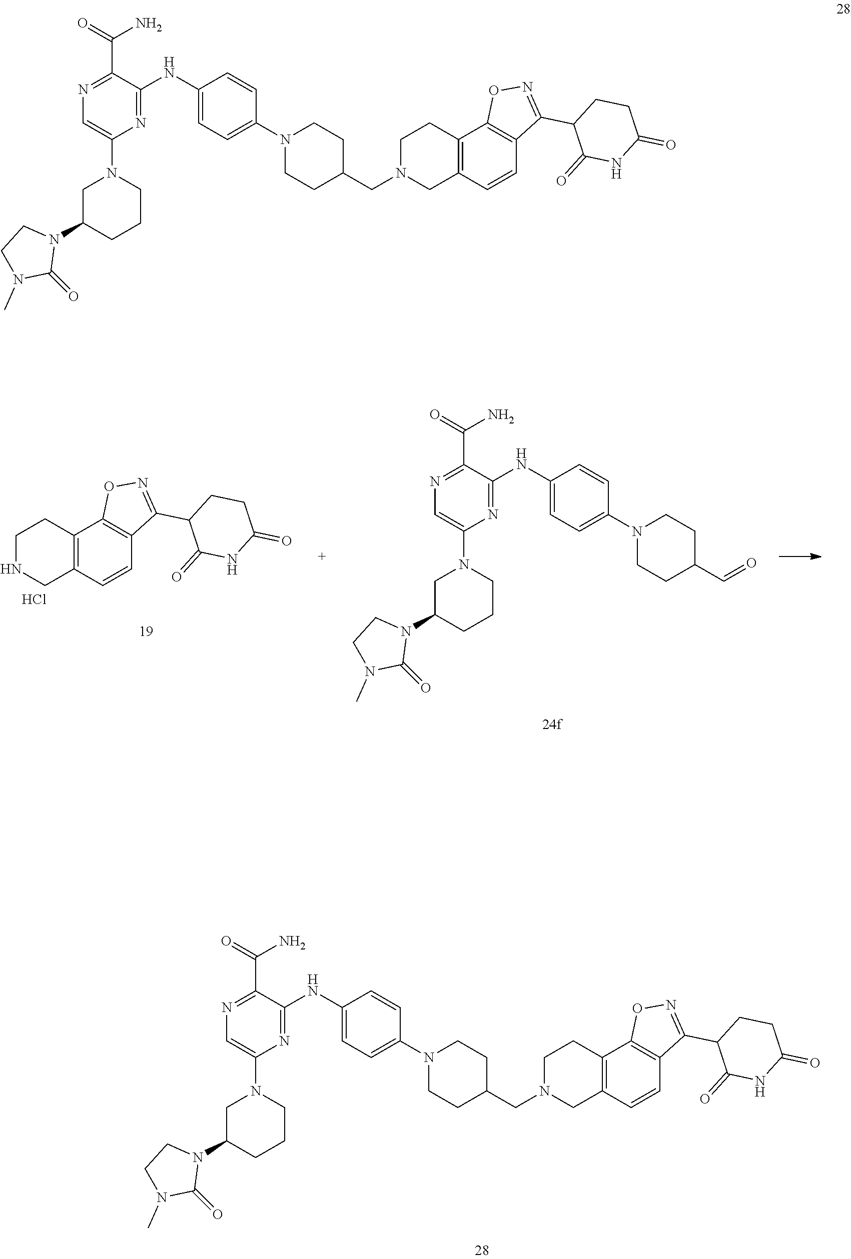 [SMILES: CN1CCN([C@@H]2CCCN(c3cnc(C(N)=O)c(Nc4ccc(N5CCC(CN6CCc7c(ccc8c(C9CCC(=O)NC9=O)noc78)C6)CC5)cc4)n3)C2)C1=O ; O=C1CCC(c2noc3c4c(ccc23)CNCC4)C(=O)N1 ; Cl ; CN1CCN([C@@H]2CCCN(c3cnc(C(N)=O)c(Nc4ccc(N5CCC(C=O)CC5)cc4)n3)C2)C1=O ; CN1CCN([C@@H]2CCCN(c3cnc(C(N)=O)c(Nc4ccc(N5CCC(CN6CCc7c(ccc8c(C9CCC(=O)NC9=O)noc78)C6)CC5)cc4)n3)C2)C1=O]

Compound 19 (107 mg), intermediate 24f (60 mg), acetic acid (6.31 mg, 6.02 μL, 0.105 mmol), 1,2-dichloroethane (5 mL), and isopropanol (2 mL) were added to a reaction flask in sequence. After the mixture was stirred and reacted at room temperature for 0.5 h, sodium cyanoborohydride (39.6 mg) was added, and the mixture was stirred and reacted at room temperature for 1 h. After the reaction was completed, 2 mL of a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography (DCM:MeOH=10:1, v/v) to give compound 28 (96 mg).

MS(ESI, [M+H]⁺) m/z: 776.40.

¹H NMR (500 MHz, DMSO-d6) δ 11.09 (s, 1H), 10.98 (s, 1H), 7.71 (d, J=3.0 Hz, 1H), 7.58 (d, J=14.0 Hz, 2H), 7.41 (d, J=8.5 Hz, 2H), 7.28 (d, J=2.9 Hz, 1H), 7.11 (d, J=8.2 Hz, 1H), 6.87 (d, J=8.5 Hz, 2H), 4.56 (dd, J=11.9, 5.0 Hz, 1H), 4.36 (d, J=12.5 Hz, 1H), 4.26 (d, J=13.3 Hz, 1H), 3.70 (s, 2H), 3.65-3.55 (m, 3H), 3.27 (ddd, J=28.5, 15.0, 7.7 Hz, 4H), 3.05-2.88 (m, 4H), 2.77 (p, J=6.4 Hz, 3H), 2.69 (s, 3H), 2.66-2.57 (m, 3H), 2.49-2.44 (m, 1H), 2.40 (d, J=6.8 Hz, 2H), 2.19 (dt, J=13.2, 4.7 Hz, 1H), 1.88-1.69 (m, 6H), 1.61-1.49 (m, 1H), 1.27 (q, J=8.8, 5.1 Hz, 2H).

### Example 29: Synthesis of Compound 29

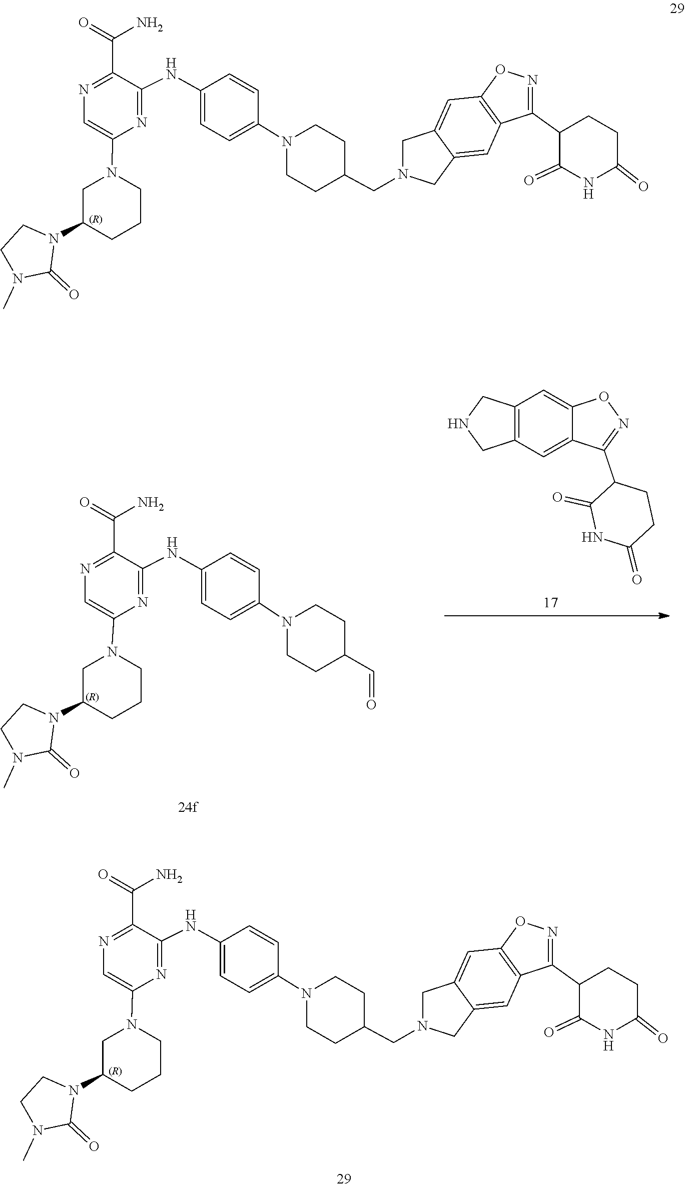

### Step 1: Preparation of Compound 29

Intermediate 24f (118 mg), 17 (100 mg), sodium acetate (21.2 mg), and dichloroethane/isopropanol (5:1, 20 mL) were added to a reaction flask in sequence. After the mixture was stirred at room temperature for 30 min sodium cyanoborohydride (32.6 mg) was added, and the mixture was reacted at room temperature for 1 h. 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 29 (60 mg).

MS(ESI, [M+H]⁺) m/z: 762.5.

1H NMR (500 MHz, DMSO-d6) 11.09 (s, 1H), 10.98 (s, 1H), 7.71 (d, J=2.9 Hz, 1H), 7.65 (s, 1H), 7.60 (d, J=2.9 Hz, 2H), 7.45-7.38 (m, 2H), 7.28 (d, J=2.9 Hz, 1H), 6.94-6.83 (m, 2H), 4.55 (dd, J=11.9, 5.0 Hz, 1H), 4.45-4.20 (m, 2H), 3.91 (d, J=25.4 Hz, 4H), 3.60 (dd, J=9.9, 5.9 Hz, 3H), 3.32-3.21 (m, 4H), 2.96 (dt, J=28.1, 11.7 Hz, 2H), 2.77 (ddd, J=17.3, 12.0, 5.4 Hz, 1H), 2.70 (s, 3H), 2.67-2.56 (m, 5H), 2.53 (d, J=4.6 Hz, 1H), 2.19 (dq, J=13.6, 4.9 Hz, 1H), 1.93-1.71 (m, 5H), 1.70-1.49 (m, 2H), 1.35-1.26 (m, 2H).

### Example 30: Synthesis of Compound 30

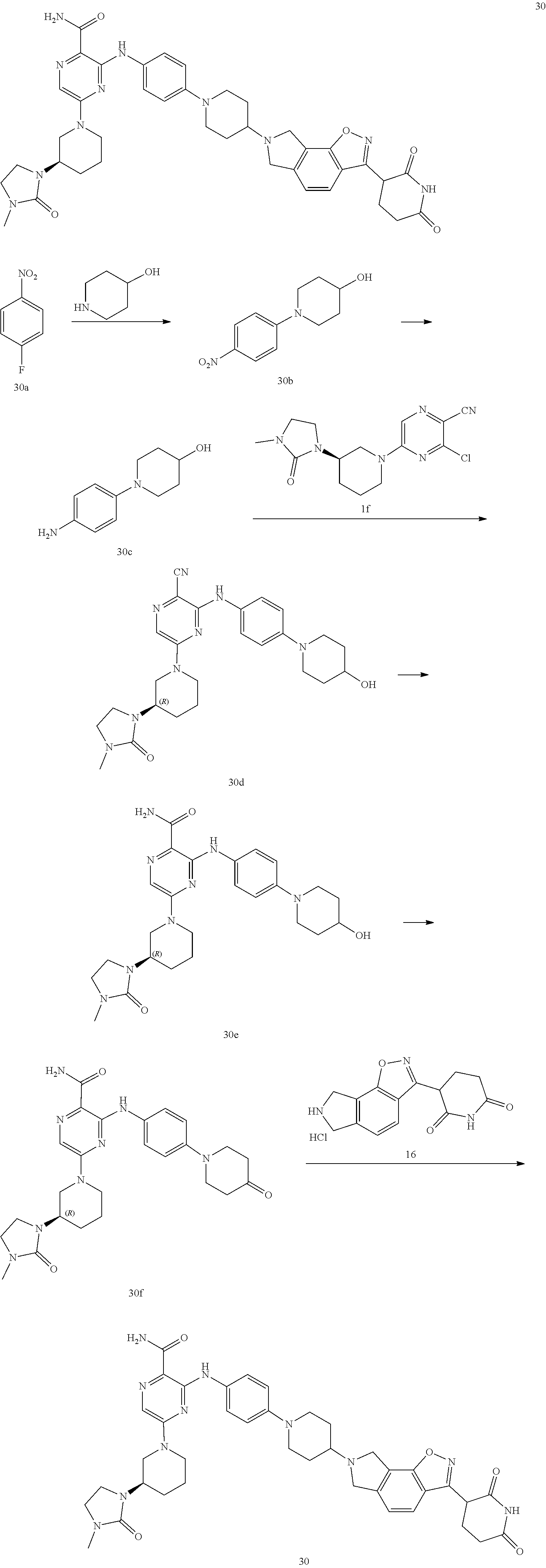

### Step 1: Preparation of Intermediate 30b

30a (6.5 g), DMSO (50 mL), piperidin-4-ol (5.13 g, 50.7 mmol), and N,N-diisopropylethylamine (17.86 g, 138 mmol) were added to a reaction flask in sequence, and the mixture was heated to 100° C. and reacted. The reaction solution was poured into ice water and filtered under vacuum, and the filter cake was dried to give 30b (10.13 g).

MS(ESI, [M+H]+) nm/z: 223.2.

### Step 2: Preparation of Intermediate 30c

30b (10 g), 10% palladium on carbon (0.3 g, 0.282 mmol), and methanol (100 mL) were added to a reaction flask in sequence, and the mixture was purged 3 times with hydrogen and reacted at room temperature under hydrogen atmosphere. After the reaction was completed, as confirmed by TLC, palladium on carbon was removed by filtration under vacuum, and the filtrate was concentrated to give 30c (8.45 g).

¹H NMR (500 MHz, DMSO-d6) δ 6.70-6.62 (m, 2H), 6.53-6.37 (m, 2H), 4.59 (d, J=4.2 Hz, 1H), 4.51 (s, 2H), 3.52 (tq, J=8.5, 4.1 Hz, 1H), 3.20 (ddd, J=12.4, 6.4, 2.7 Hz, 2H), 2.58 (ddd, J=12.6, 10.4, 2.9 Hz, 2H), 1.84-1.70 (m, 2H), 1.47 (dtd, J=13.0, 9.7, 3.8 Hz, 2H).

### Step 3: Preparation of Intermediate 30d

Intermediate 1f (5 g), 30c (3.00 g), cesium carbonate (15.24 g), BINAP (0.971 g), palladium acetate (0.350 g), and 1,4-dioxane (100 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 30d (6.6 g).

¹H NMR (500 MHz, DMSO-4) δ 8.78 (s, 1H), 7.75 (s, 1H), 7.40-7.29 (m, 2H), 7.00-6.78 (m, 2H), 4.66 (d, J=−4.2 Hz, 1H), 4.41-4.13 (m, 2H), 3.71-3.49 (m, 2H), 3.44 (dt, J=12.3, 4.3 Hz, 2H), 3.31-3.19 (m, 4H), 3.00-2.83 (m, 2H), 2.77 (ddd, J=12.6, 10.1, 2.9 Hz, 2H), 2.70 (s, 3H), 1.84-1.68 (m, 5H), 1.48 (tdd, J=12.8, 9.3, 5.9 Hz, 3H).

### Step 4: Preparation of Intermediate 30c

Intermediate 30d (6.6 g), DMSO (25 mL), and cesium carbonate (4.08 g) were added to a single-necked flask in sequence. 30 wt % hydrogen peroxide (4.24 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 30e (6.4 g).

MS(ESI, [M+H]+) m/z: 495.19

### Step 5: Preparation of Intermediate 30f

N,N-diisopropylethylamine (8.49 g, 65.7 mmol) was added to a stirred solution of 30e (6.5 g) in dichloromethane (60 mL) under an ice bath, and then a solution of sulfur trioxide pyridine (6.27 g) in DMSO (5 mL) was added. The mixture was reacted at room temperature. After the reaction was completed, 300 mL of a saturated sodium sulfite solution was added to the reaction solution to quench the reaction, and ethyl acetate was added for extraction. The organic phase was dried over anhydrous sodium sulfate and concentrated by evaporation at reduced pressure to remove the solvent, thus giving 30f (5.9 g).

MS(ESI, [M+H]+) m/z: 492.47.

### Step 6: Preparation of Compound 30

30f (0.15 g), isopropanol (1 mL), 1,2-dichloroethane (5.00 mL), and 16 (0.141 g) were added to a reaction flask in sequence, and sodium cyanoborohydride (0.057 g) was added. The mixture was reacted at room temperature. After the reaction was completed, the reaction solution was purified by silica gel column chromatography to give 30 (15 mg).

MS(ESI, [M+H]+) m/z: 748.35.

¹H NMR (500 MHz, DMSO-d6) δ 11.10 (s, 1H), 10.98 (s, 1H), 7.72 (d, J=8.3 Hz, 2H), 7.60 (s, 1H), 7.43 (d, J=8.4 Hz, 2H), 7.32 (d, J=8.2 Hz, 1H), 7.29-7.22 (m, 1H), 6.91 (d, J=8.6 Hz, 2H), 4.60 (dd, J=11.9, 5.0 Hz, 1H), 4.36 (d, J=12.4 Hz, 1H), 4.27 (d, J=13.5 Hz, 1H), 4.22 (s, 2H), 4.11 (s, 2H), 3.58 (d, J=12.1 Hz, 3H), 3.27 (dq, J=15.0, 8.1, 7.2 Hz, 3H), 2.97 (dt, J=32.4, 12.1 Hz, 2H), 2.77 (t, J=11.8 Hz, 3H), 2.71 (m, 4H), 2.61 (dt, J=17.3, 4.5 Hz, 2H), 2.57-2.52 (m, 1H), 2.25-2.14 (m, 1H), 2.04 (d, J=12.3 Hz, 2H), 1.86-1.71 (m, 3H), 1.70-1.50 (m, 3H).

### Example 31: Synthesis of Compound 31

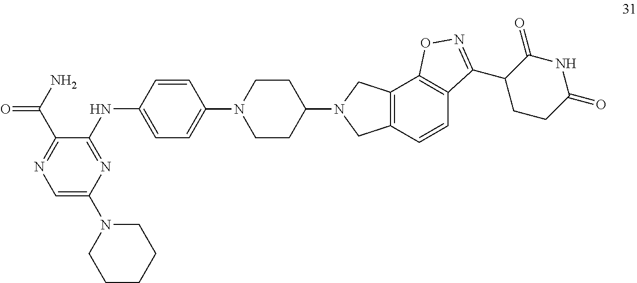 [SMILES: NC(=O)c1ncc(N2CCCCC2)nc1Nc1ccc(N2CCC(N3Cc4ccc5c(C6CCC(=O)NC6=O)noc5c4C3)CC2)cc1]

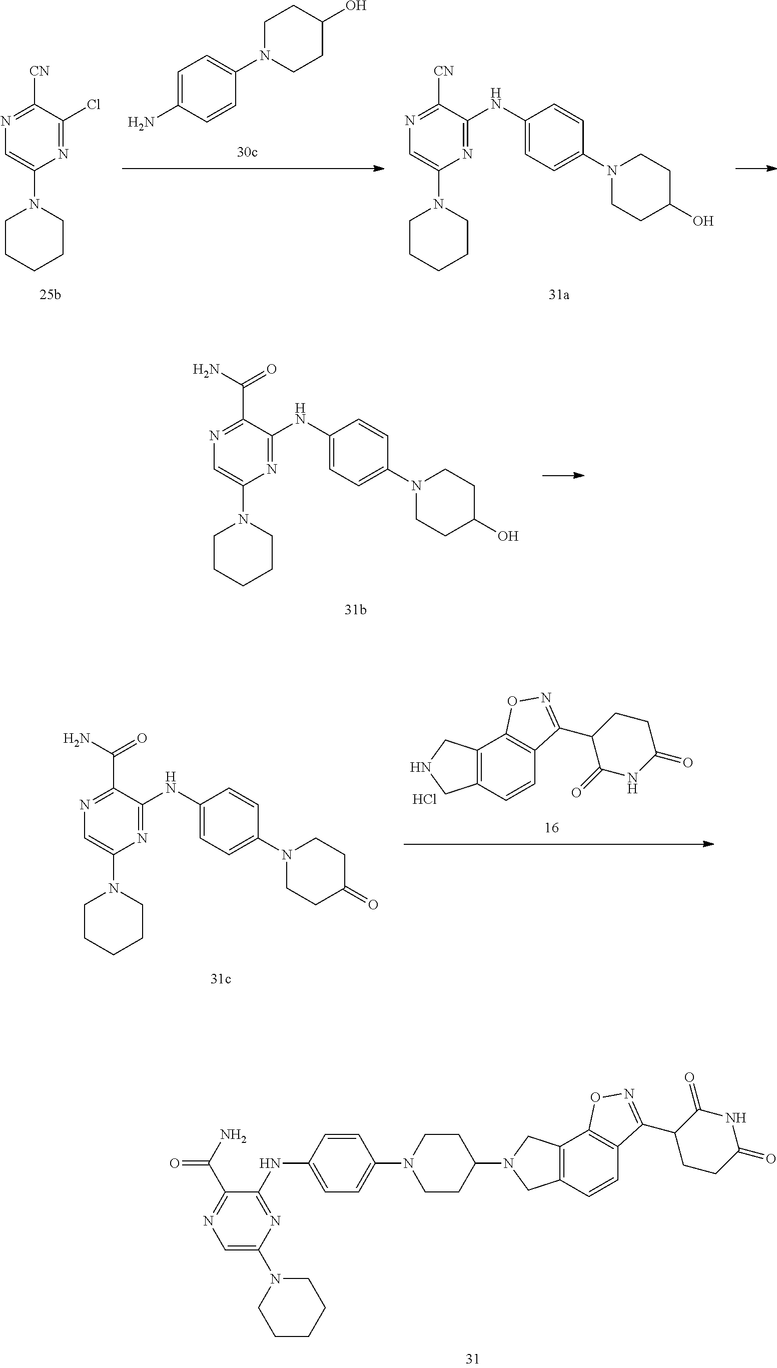

### Step 1: Preparation of Intermediate 31a

Intermediate 25b (1 g), 30c (0.859 g), cesium carbonate (4.39 g), BINAP (0.280 g), palladium acetate (0.101 g), and 1,4-dioxane (10 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 31a (0.85 g).

MS(ESI, [M+H]+) m/z: 378.43.

### Step 2: Preparation of Intermediate 31b

Intermediate 31a (2.8 g), DMSO (10 mL), and cesium carbonate (2.4 g) were added to a single-necked flask in sequence. 30 wt % hydrogen peroxide (2.52 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 31b (2.85 g).

MS(ESI, [M+H]+) m/z: 396.37.

### Step 3: Preparation of Intermediate 31c

N,N-diisopropylethylamine (2.54 g) was added to a stirred solution of 31b (2.6 g) in dichloromethane (30 mL) under an ice bath, and then a solution of sulfur trioxide pyridine (1.56 g) in DMSO (5 mL) was added. The mixture was reacted at room temperature. After the reaction was completed, 300 mL of a saturated sodium sulfite solution was added to the reaction solution to quench the reaction, and 200 mL of ethyl acetate was added for extraction. The organic phase was dried over anhydrous sodium sulfate and concentrated by evaporation at reduced pressure to remove the solvent, thus giving 31c (1.85 g).

¹H NMR (500 MHz, DMSO-d6) δ 11.11 (s, 1H), 7.70 (d, J=2.9 Hz, 1H), 7.61 (s, 1H), 7.52-7.43 (m, 2H), 7.27 (d, J=3.0 Hz, 1H), 7.06-6.96 (m, 2H), 3.66 (t, J=5.4 Hz, 4H), 3.53 (t, J=6.0 Hz, 4H), 2.42 (t, J=6.0 Hz, 4H), 1.62 (dq, J=34.2, 5.9 Hz, 6H).

### Step 4: Preparation of Compound 31

31c (0.1 g), isopropanol (1 mL), 1,2-dichloroethane (5.00 mL), and 16 (0.117 g) were added to a reaction flask in sequence, and sodium cyanoborohydride (0.048 g) was added. The mixture was reacted at room temperature. After the reaction was completed, the reaction solution was purified by silica gel column chromatography to give 3l (60 mg).

MS(ESI, [M+H]+) m/z: 649.44.

¹H NMR (500 MHz, DMSO-d6) δ 11.08 (d, J=15.0 Hz, 2H), 7.72 (d, J=8.0 Hz, 1H), 7.69 (d, J=2.9 Hz, 1H), 7.60 (s, 1H), 7.47-7.41 (m, 2H), 7.32 (d, J=8.2 Hz, 1H), 7.26 (d, J=2.9 Hz, 1H), 6.97-6.92 (m, 2H), 4.60 (dd, J=11.9, 5.0 Hz, 1H), 4.22 (s, 2H), 4.11 (s, 2H), 3.66 (t, J=5.5 Hz, 4H), 3.59 (d, J=12.0 Hz, 2H), 2.83-2.72 (m, 3H), 2.68-2.58 (m, 2H), 2.54 (dd, J=10.0, 3.6 Hz, 1H), 2.26-2.15 (m, 1H), 2.04 (d, J=12.2 Hz, 2H), 1.73-1.50 (m, 8H).

### Examples 32 and 33: Synthesis of Compounds 32 and 33

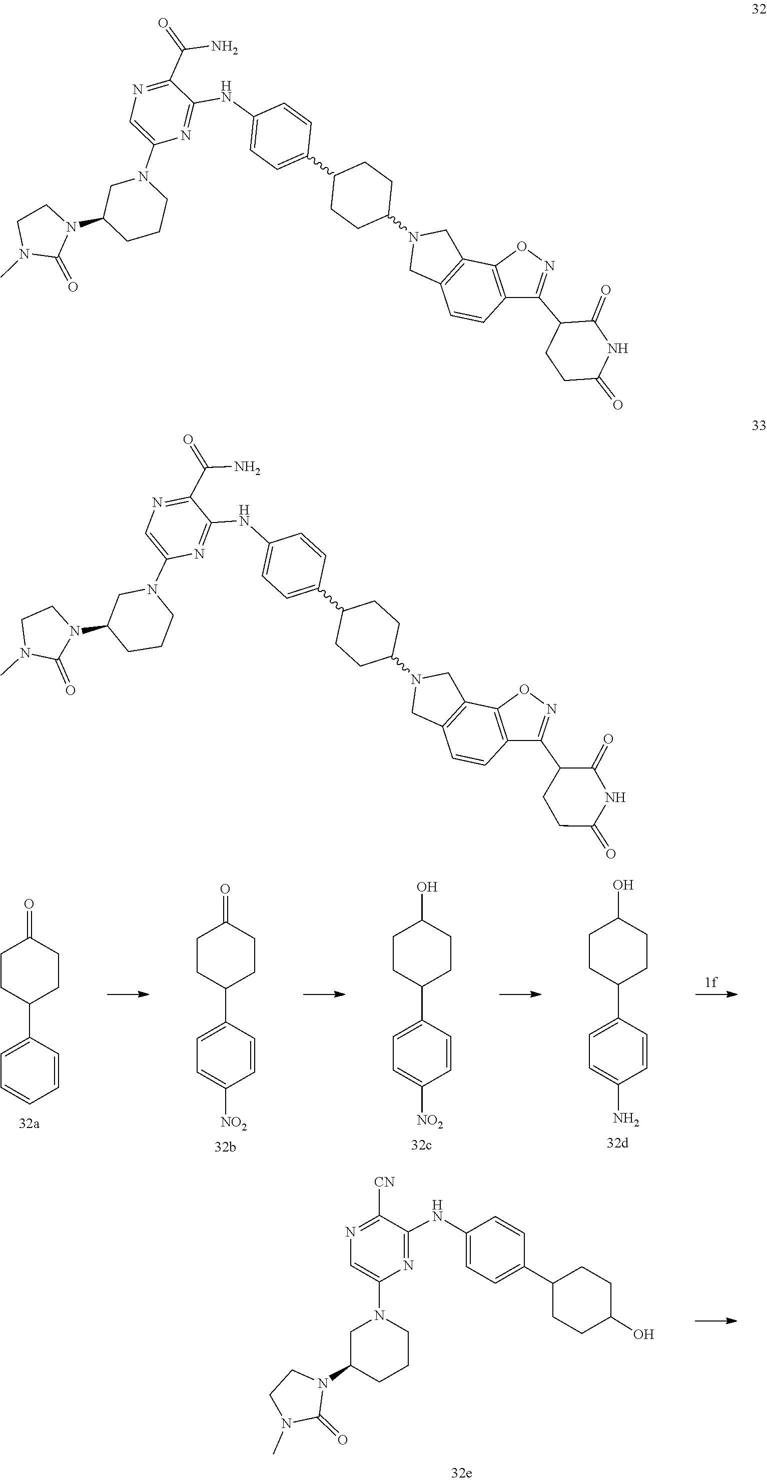

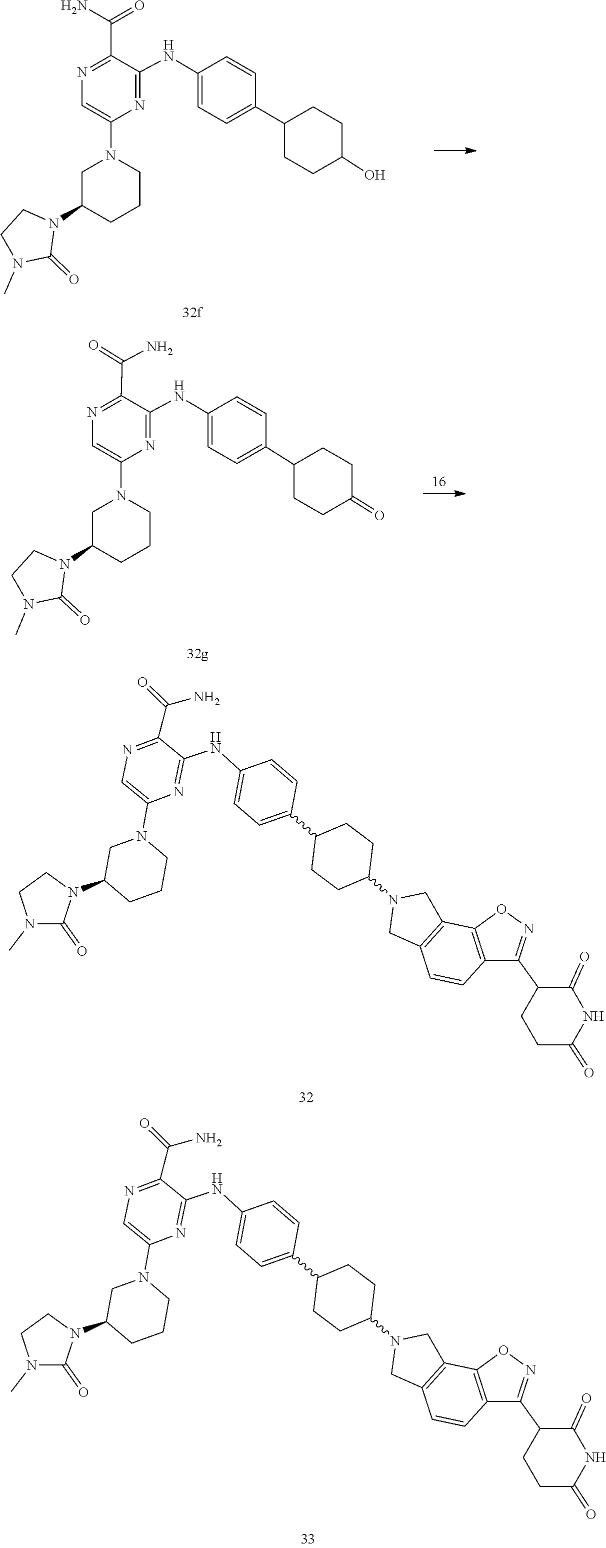 [SMILES: CN1CCN([C@@H]2CCCN(c3cnc(C(N)=O)c(Nc4ccc(C5CCC(O)CC5)cc4)n3)C2)C1=O ; CN1CCN([C@@H]2CCCN(c3cnc(C(N)=O)c(Nc4ccc(C5CCC(=O)CC5)cc4)n3)C2)C1=O ; CN1CCN([C@@H]2CCCN(c3cnc(C(N)=O)c(Nc4ccc(C5CCC(N6Cc7ccc8c(C9CCC(=O)NC9=O)noc8c7C6)CC5)cc4)n3)C2)C1=O ; CN1CCN([C@@H]2CCCN(c3cnc(C(N)=O)c(Nc4ccc(C5CCC(N6Cc7ccc8c(C9CCC(=O)NC9=O)noc8c7C6)CC5)cc4)n3)C2)C1=O]

### Step 1: Preparation of Intermediate 32b

32a (10 g), dichloromethane (100 mL), and sulfuric acid (11.26 g, 6.15 mL, 115 mmol) were added to a reaction flask in sequence, and nitric acid (4.70 g, 3.11 mL, 74.6 mmol) was added under an ice bath. The mixture was reacted at room temperature. After the reaction was completed, the reaction solution was added to a saturated sodium bicarbonate solution to quench the reaction, and 200 mL of ethyl acetate was added for extraction. The organic phase was dried over anhydrous sodium sulfate, filtered, and concentrated to give a crude product, and the crude product was slurried with a small amount of methyl tert-butyl ether and filtered under vacuum to give 32b (7.43 g).

¹H NMR (500 MHz, DMSO-d6) δ 8.24-8.14 (m, 2H), 7.64-7.57 (m, 2H), 3.25 (tt, J=12.1, 3.4 Hz, 1H), 2.61 (td, J=14.2, 6.0 Hz, 2H), 2.29 (ddt, J=14.6, 4.3, 2.1 Hz, 2H), 2.09 (ddd, J=12.8, 6.1, 3.1 Hz, 2H), 1.94 (qd, J=13.0, 4.2 Hz, 2H).

### Step 2: Preparation of Intermediate 32c

30b (7.4 g), MeOH (70 mL), and sodium borohydride (1.277 g) were added to a reaction flask in sequence under an ice bath, and the mixture was reacted at room temperature. After the reaction was completed, the reaction solution was added to a saturated ammonium chloride solution to quench the reaction, and 200 mL of ethyl acetate was added for extraction. The organic phase was dried over anhydrous sodium sulfate, filtered, and concentrated to give 32c (6.05 g).

¹H NMR (500 MHz, DMSO-d6) δ 8.20-8.11 (m, 2H), 7.52 (dd, J=8.8, 1.9 Hz, 2H), 4.61 (dd, J=4.4, 1.8 Hz, 1H), 3.48 (ddt, J=10.9, 6.6, 4.5 Hz, 1H), 2.63 (tt, J=12.1, 3.4 Hz, 1H), 1.99-1.89 (m, 2H), 1.83-1.75 (m, 2H), 1.51 (qd, J=13.0, 11.0, 3.4 Hz, 2H), 1.37-1.25 (m, 2H).

### Step 3: Preparation of Intermediate 32d

32c (6 g), iron powder (4.54 g), ammonium chloride (6 g), MeOH (60 mL), and 20 mL of water were added to a reaction flask in sequence, and the mixture was warmed to 80° C. and reacted. After the reaction was completed, the solid was removed by filtration under vacuum, 200 mL of ethyl acetate was added to the filtrate, and 200 mL of a saturated sodium carbonate solution was added for extraction. The organic phase was dried over anhydrous sodium sulfate, filtered, and concentrated to give 32d (2.35 g).

¹H NMR (500 MHz, DMSO-d6) δ 6.93-6.80 (m, 2H), 6.54-6.41 (m, 2H), 4.77 (s, 2H), 4.50 (d, J=4.4 Hz, 1H), 3.40 (ddt, J=15.0, 10.7, 4.3 Hz, 1H), 2.24 (tt, J=12.0, 3.5 Hz, 1H), 1.92-1.81 (m, 2H), 1.68 (dt, J=12.8, 2.9 Hz, 2H), 1.43-1.30 (m, 2H), 1.30-1.19 (m, 2H).

### Step 4: Preparation of Intermediate 32e

Intermediate 1f (1 g), 32d (0.775 g), cesium carbonate (3.05 g), BINAP (0.194 g), palladium acetate (0.070 g), and 1,4-dioxane (10 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 32e (1.31 g).

MS(ESI, [M+H]+) m/z: 476.50.

### Step 5: Preparation of Intermediate 32f

Intermediate 32e (1.3 g), DMSO (5 mL), and cesium carbonate (0.891 g) were added to a single-necked flask in sequence. 30 wt % hydrogen peroxide (0.930 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 32f (1.21 g).

MS(ESI, [M+H]⁺) m/z: 494.47.

### Step 6: Preparation of Intermediate 32g

32f (1.5 g), IBX (1.702 g), and DMSO (15 mL) were added to a reaction flask in sequence, and the mixture was reacted at room temperature. After the reaction was completed, 200 mL of ethyl acetate was added, and 200 mL of a saturated sodium bicarbonate solution was added for extraction. The organic phase was dried over anhydrous sodium sulfate, filtered, and concentrated to give 32g (1.02 g).

MS(ESI, [M+H]+) m/z: 492.40.

### Step 7: Preparation of Compounds 32 and 33

32g (200 mg), isopropanol (1 mL), 1,2-dichloroethane (5.00 mL), and 16 (188 mg) were added to a reaction flask in sequence, and sodium cyanoborohydride (77 mg) was added. The mixture was reacted at room temperature. After the reaction was completed, the reaction solution was purified by silica gel column chromatography (polarity of developing solvents: MeOH:DCM=0:100-1:25) to give 32 (21 mg) and 33 (26 mg) in sequence. 32: MS(ESI, [M+H]+) m/z: 747.39.

¹H NMR (500 MHz, DMSO-d6) δ 11.16 (s, 1H), 11.10 (s, 1H), 7.78-7.69 (m, 2H), 7.63 (s, 1H), 7.46 (d, J=8.2 Hz, 2H), 7.39-7.28 (m, 2H), 7.12 (d, J=8.3 Hz, 2H), 4.61 (dd, J=11.9, 4.9 Hz, 1H), 4.44-4.33 (m, 1H), 4.24 (d, J=13.5 Hz, 1H), 4.17 (s, 2H), 4.06 (s, 2H), 3.56 (dq, J=11.0, 5.6, 4.3 Hz, 1H), 3.26 (dd, J=9.3, 6.9 Hz, 2H), 3.05 (t, J=7.7 Hz, 2H), 2.94 (q, J=13.3 Hz, 2H), 2.78 (dq, J=17.1, 5.7, 5.2 Hz, 2H), 2.66-2.60 (m, 1H), 2.60-2.53 (m, 2H), 2.45 (s, 3H), 2.27-2.15 (m, 1H), 2.06 (d, J=13.3 Hz, 2H), 1.82 (dt, J=37.4, 12.5 Hz, 4H), 1.68 (dt, J=26.8, 12.6 Hz, 3H), 1.54 (t, J=14.8 Hz, 3H). 33: MS(ESI, [M+H]+) m/z: 747.55.

¹H NMR (500 MHz, DMSO-d6) δ 11.20 (s, 1H), 11.10 (s, 1H), 7.76 (d, J=2.9 Hz, 1H), 7.72 (d, J=8.0 Hz, 1H), 7.66 (s, 1H), 7.50 (d, J=8.2 Hz, 2H), 7.36-7.29 (m, 2H), 7.17 (d, J=8.2 Hz, 2H), 4.60 (dd, J=11.9, 5.0 Hz, 1H), 4.32 (dd, J=33.0, 12.7 Hz, 2H), 4.23 (s, 2H), 4.12 (s, 2H), 3.62 (ddt, J=11.2, 8.6, 4.2 Hz, 1H), 3.38-3.33 (m, 2H), 3.30-3.22 (m, 2H), 3.00 (dt, J=40.8, 11.5 Hz, 2H), 2.82-2.68 (m, 4H), 2.66-2.59 (m, 1H), 2.58-2.51 (m, 2H), 2.47 (d, J=11.2 Hz, 1H), 2.26-2.11 (m, 3H), 1.93-1.72 (m, 5H), 1.48 (dp, J=49.1, 12.0 Hz, 5H).

### Example 34: Synthesis of Compound 34

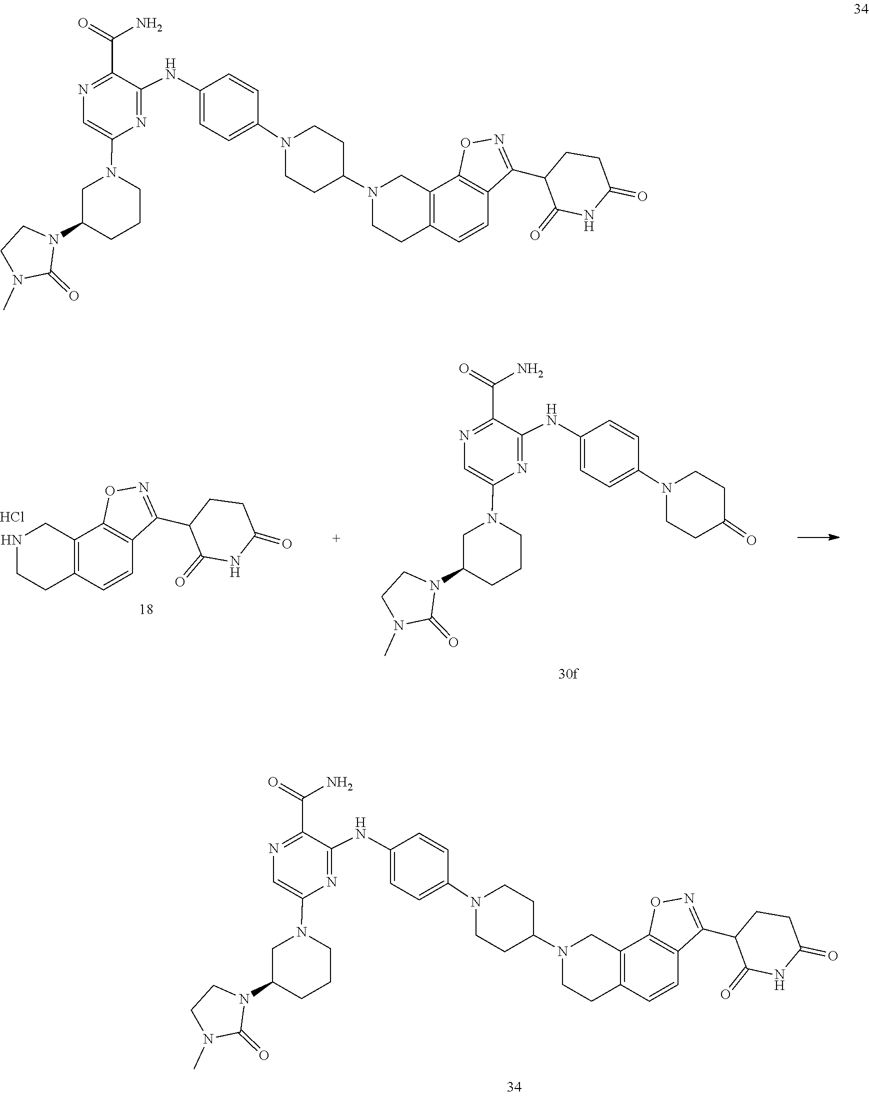 [SMILES: CN1CCN([C@@H]2CCCN(c3cnc(C(N)=O)c(Nc4ccc(N5CCC(N6CCc7ccc8c(C9CCC(=O)NC9=O)noc8c7C6)CC5)cc4)n3)C2)C1=O ; Cl ; O=C1CCC(c2noc3c4c(ccc23)CCNC4)C(=O)N1 ; CN1CCN([C@@H]2CCCN(c3cnc(C(N)=O)c(Nc4ccc(N5CCC(=O)CC5)cc4)n3)C2)C1=O ; CN1CCN([C@@H]2CCCN(c3cnc(C(N)=O)c(Nc4ccc(N5CCC(N6CCc7ccc8c(C9CCC(=O)NC9=O)noc8c7C6)CC5)cc4)n3)C2)C1=O]

Compound 18 (116 mg), intermediate 30f (200 mg), acetic acid (12.21 mg, 0.012 mL, 0.203 mmol), 1,2-dichloroethane (10 mL), and isopropanol (3 mL) were added to a reaction flask in sequence. After the mixture was stirred and reacted at room temperature for 0.5 h, sodium cyanoborohydride (77 mg, 1.220 mmol) was added, and the mixture was stirred and reacted at room temperature. After the reaction was completed, as confirmed by TLC, 2 mL of a saturated sodium bicarbonate solution was added to the reaction solution to neutralize acetic acid, and then 50 mL of dichloromethane and 100 mL were added for extraction. The organic phase was separated, and the aqueous phase was extracted twice with 20 mL of dichloromethane. The organic phases were combined, washed with 100 mL of saturated brine, dried over anhydrous sodium sulfate, and filtered, and the filtrate was purified by silica gel column chromatography to give compound 34 (84 mg).

MS(ESI, [M+H]⁺) m/z: 761.55.

¹H NMR (500 MHz, DMSO-d6) δ 11.09 (s, 1H), 10.99 (s, 1H), 7.71 (d, J=2.9 Hz, 1H), 7.59 (d, J=14.5 Hz, 2H), 7.43 (d, J=8.7 Hz, 2H), 7.28 (d, J=3.0 Hz, 1H), 7.14 (d, J=8.2 Hz, 1H), 6.91 (d, J=8.7 Hz, 2H), 4.56 (dd, J=11.9, 5.0 Hz, 1H), 4.41-4.32 (m, 1H), 4.27 (d, J=13.3 Hz, 1H), 3.98 (s, 2H), 3.68 (d, J=11.7 Hz, 2H), 3.60 (dq, J=8.1, 5.3, 4.1 Hz, 1H), 3.32-3.20 (m, 4H), 3.02-2.85 (m, 6H), 2.80-2.74 (m, 1H), 2.71 (s, 3H), 2.70-2.65 (m, 2H), 2.60 (dt, J=17.3, 4.3 Hz, 2H), 2.47 (dd, J=12.3, 4.5 Hz, 1H), 2.19 (dq, J=13.7, 4.8 Hz, 1H), 1.97 (d, J=11.9 Hz, 2H), 1.81 (d, J=11.4 Hz, 2H), 1.78-1.63 (m, 3H), 1.59-1.50 (m, 1H).

### Example 35: Synthesis of Compound 35

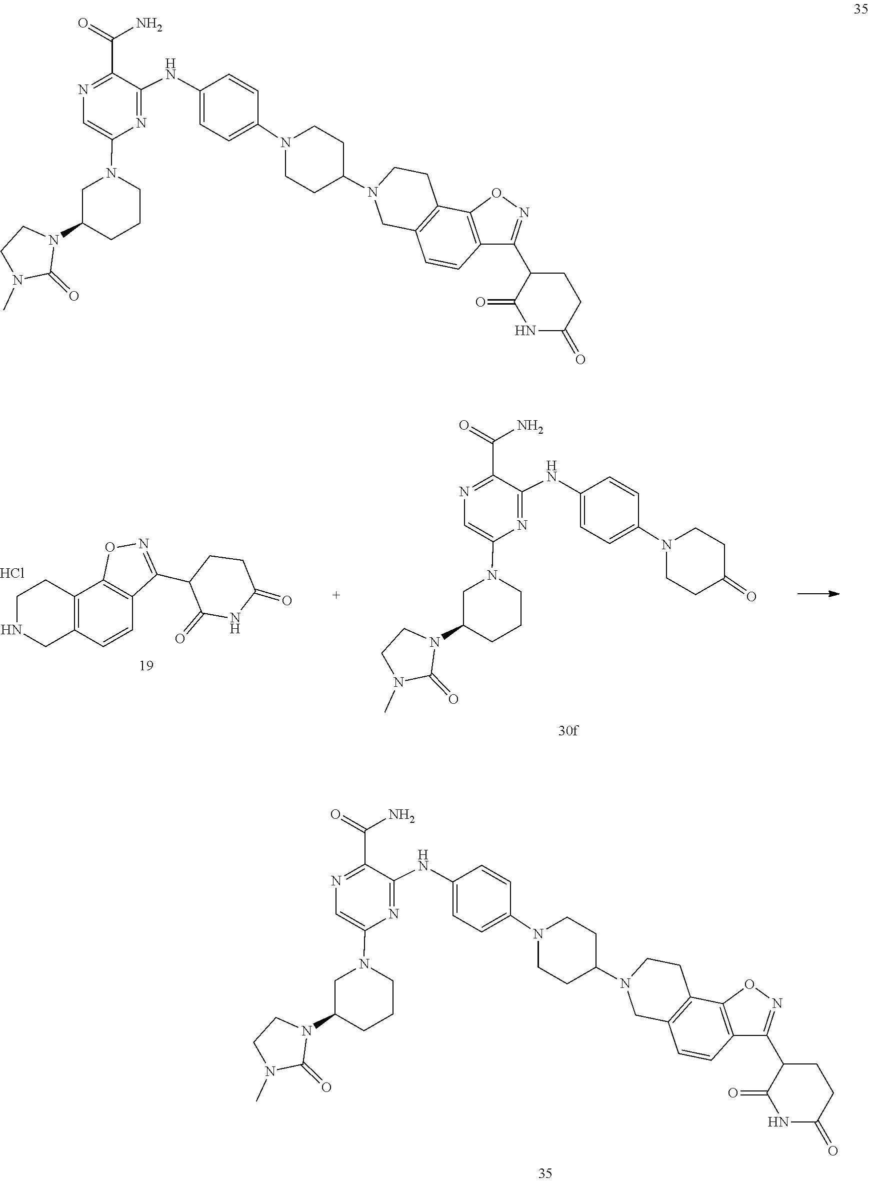 [SMILES: CN1CCN([C@@H]2CCCN(c3cnc(C(N)=O)c(Nc4ccc(N5CCC(N6CCc7c(ccc8c(C9CCC(=O)NC9=O)noc78)C6)CC5)cc4)n3)C2)C1=O ; Cl ; O=C1CCC(c2noc3c4c(ccc23)CNCC4)C(=O)N1 ; CN1CCN([C@@H]2CCCN(c3cnc(C(N)=O)c(Nc4ccc(N5CCC(=O)CC5)cc4)n3)C2)C1=O ; CN1CCN([C@@H]2CCCN(c3cnc(C(N)=O)c(Nc4ccc(N5CCC(N6CCc7c(ccc8c(C9CCC(=O)NC9=O)noc78)C6)CC5)cc4)n3)C2)C1=O]

Compound 19 (113 mg), intermediate 30f (195 mg), acetic acid (71.4 mg, 0.068 mL, 1.188 mmol), 1,2-dichloroethane (10 mL), and isopropanol (3 mL) were added to a reaction flask in sequence. After the mixture was stirred and reacted at room temperature for 0.5 h, sodium cyanoborohydride (74.7 mg, 1.188 mmol) was added, and the mixture was stirred and reacted at room temperature for 1 h. After the reaction was completed, 2 mL of a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 35 (80 mg).

MS(ESI, [M+H]⁺) m/z: 761.44.

¹H NMR (500 MHz, DMSO-d6) δ 11.08 (s, 1H), 10.99 (s, 1H), 7.71 (d, J=2.9 Hz, 1H), 7.58 (d, J=21.1 Hz, 2H), 7.43 (d, J=8.6 Hz, 2H), 7.28 (d, J=3.0 Hz, 1H), 7.12 (d, J=8.3 Hz, 1H), 6.90 (d, J=8.6 Hz, 2H), 4.56 (dd, J=11.8, 5.0 Hz, 1H), 4.36 (d, J=12.5 Hz, 1H), 4.27 (d, J=13.3 Hz, 1H), 3.88 (s, 2H), 3.67 (d, J=11.7 Hz, 2H), 3.61 (dq, J=8.0, 5.3, 4.1 Hz, 1H), 3.32-3.20 (m, 4H), 3.03-2.87 (m, 6H), 2.80-2.74 (m, 1H), 2.71 (s, 3H), 2.69-2.57 (m, 4H), 2.50-2.43 (m, 1H), 2.19 (dq, J=13.6, 4.8 Hz, 1H), 1.99-1.90 (m, 2H), 1.81 (d, J=10.9 Hz, 2H), 1.75 (dd, J=11.6, 3.6 Hz, 1H), 1.69 (t, J=12.3 Hz, 2H), 1.59-1.51 (m, 1H).

### Example 36: Synthesis of Compound 36

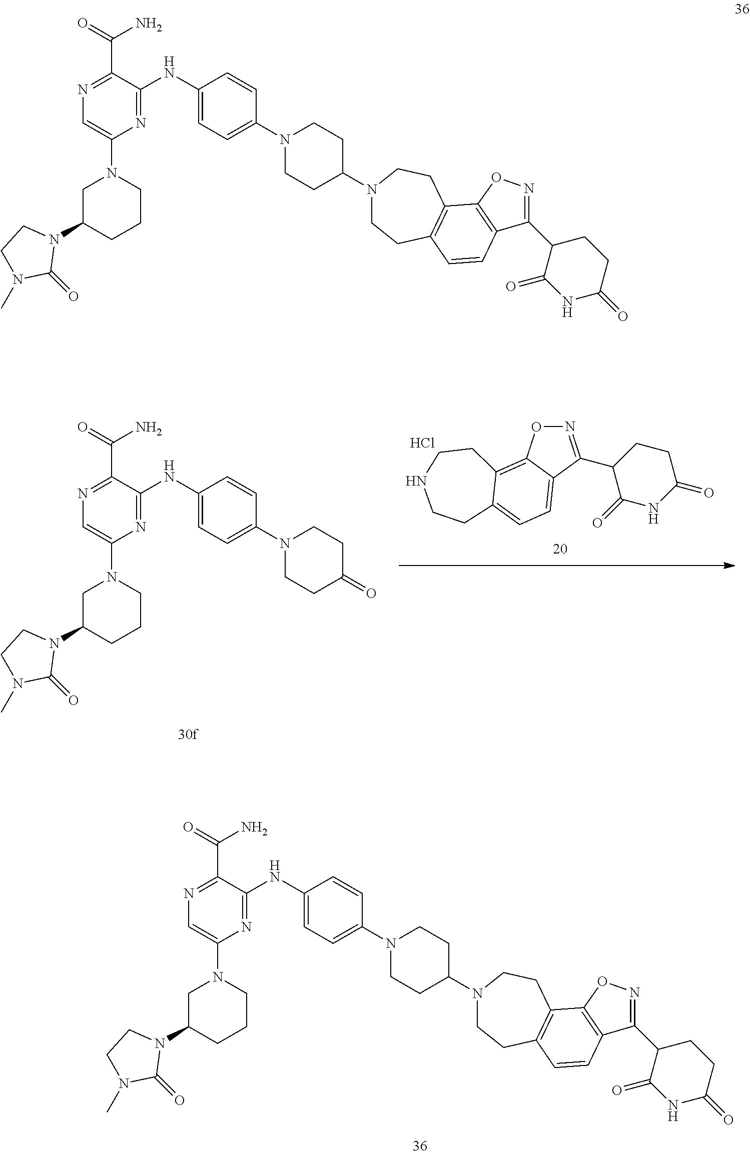 [SMILES: CN1CCN([C@@H]2CCCN(c3cnc(C(N)=O)c(Nc4ccc(N5CCC(N6CCc7ccc8c(C9CCC(=O)NC9=O)noc8c7CC6)CC5)cc4)n3)C2)C1=O ; CN1CCN([C@@H]2CCCN(c3cnc(C(N)=O)c(Nc4ccc(N5CCC(=O)CC5)cc4)n3)C2)C1=O ; Cl ; O=C1CCC(c2noc3c4c(ccc23)CCNCC4)C(=O)N1 ; CN1CCN([C@@H]2CCCN(c3cnc(C(N)=O)c(Nc4ccc(N5CCC(N6CCc7ccc8c(C9CCC(=O)NC9=O)noc8c7CC6)CC5)cc4)n3)C2)C1=O]

### Step 1: Preparation of Compound 36

30f (150 mg), isopropanol (1 mL), 1,2-dichloroethane (5.00 mL), and 20 (102 mg) were added to a reaction flask in sequence, and sodium cyanoborohydride (57 mg) was added. The mixture was reacted at 80° C. After the reaction was completed, the reaction solution was purified by silica gel column chromatography to give 36 (15 mg).

MS(ESI, [M+H]+) m/z: 776.59.

¹H NMR (500 MHz, DMSO-d6) δ 11.09 (s, 1H), 10.98 (s, 1H), 7.71 (d, J=2.9 Hz, 1H), 7.60 (s, 1H), 7.54 (d, J=8.0 Hz, 1H), 7.41 (d, J=8.6 Hz, 2H), 7.28 (d, J=2.8 Hz, 1H), 7.19 (d, J=8.1 Hz, 1H), 6.88 (d, J=8.5 Hz, 2H), 4.55 (dd, J=11.9, 5.0 Hz, 1H), 4.31 (dd, J=44.9, 11.8 Hz, 2H), 3.68-3.55 (m, 3H), 3.27 (dq, J=20.6, 8.1, 7.7 Hz, 4H), 3.19-3.10 (m, 2H), 3.03 (s, 2H), 3.00-2.87 (m, 2H), 2.75 (dq, J=22.8, 6.1, 5.7 Hz, 5H), 2.70 (s, 4H), 2.66-2.55 (m, 3H), 2.49 (s, 1H), 2.18 (dt, J=13.4, 4.6 Hz, 1H), 1.86-1.68 (m, 5H), 1.58 (dd, J=35.3, 12.4 Hz, 3H).

### Example 37: Synthesis of Compound 37

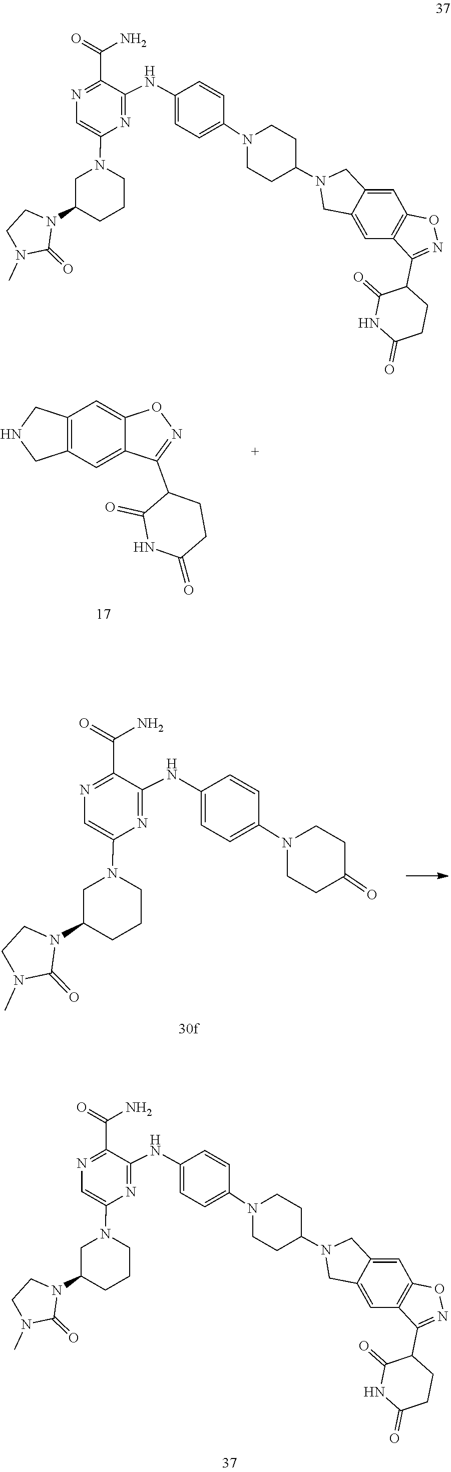 [SMILES: CN1CCN([C@@H]2CCCN(c3cnc(C(N)=O)c(Nc4ccc(N5CCC(N6Cc7cc8onc(C9CCC(=O)NC9=O)c8cc7C6)CC5)cc4)n3)C2)C1=O ; O=C1CCC(c2noc3cc4c(cc23)CNC4)C(=O)N1 ; CN1CCN([C@@H]2CCCN(c3cnc(C(N)=O)c(Nc4ccc(N5CCC(=O)CC5)cc4)n3)C2)C1=O ; CN1CCN([C@@H]2CCCN(c3cnc(C(N)=O)c(Nc4ccc(N5CCC(N6Cc7cc8onc(C9CCC(=O)NC9=O)c8cc7C6)CC5)cc4)n3)C2)C1=O]

### Step 1: Preparation of Compound 37

30f (150 mg), isopropanol (1 mL), 1,2-dichloroethane (5.00 mL), and 17 (140 mg) were added to a reaction flask in sequence, and sodium cyanoborohydride (57 mg) was added. The mixture was reacted at 80° C. After the reaction was completed, the reaction solution was purified by silica gel column chromatography to give 37 (25 mg).

MS(ESI, [M+H]+) m/z: 748.53.

¹H NMR (500 MHz, DMSO-d6) δ 11.09 (s, 1H), 10.98 (s, 1H), 7.71 (d, J=2.9 Hz, 1H), 7.67 (s, 1H), 7.61 (d, J=9.2 Hz, 2H), 7.47-7.40 (m, 2H), 7.28 (d, J=2.9 Hz, 1H), 6.95-6.87 (m, 2H), 4.56 (dd, J=11.9, 5.0 Hz, 1H), 4.31 (dd, J=44.1, 12.7 Hz, 2H), 4.02 (s, 2H), 3.96 (s, 2H), 3.67-3.52 (m, 3H), 3.34 (d, J=4.9 Hz, 1H), 3.26 (ddd, J=13.9, 11.1, 6.9 Hz, 3H), 2.97 (dt, J=33.5, 12.0 Hz, 2H), 2.77 (td, J=11.8, 11.4, 4.7 Hz, 3H), 2.71 (s, 3H), 2.65-2.52 (m, 3H), 2.19 (dq, J=13.7, 5.0 Hz, 1H), 2.00 (dd, J=10.6, 5.3 Hz, 2H), 1.85-1.71 (m, 3H), 1.59 (p, J=16.0, 13.3 Hz, 3H).

### Examples 38 and 39: Synthesis of Compounds 38 and 39

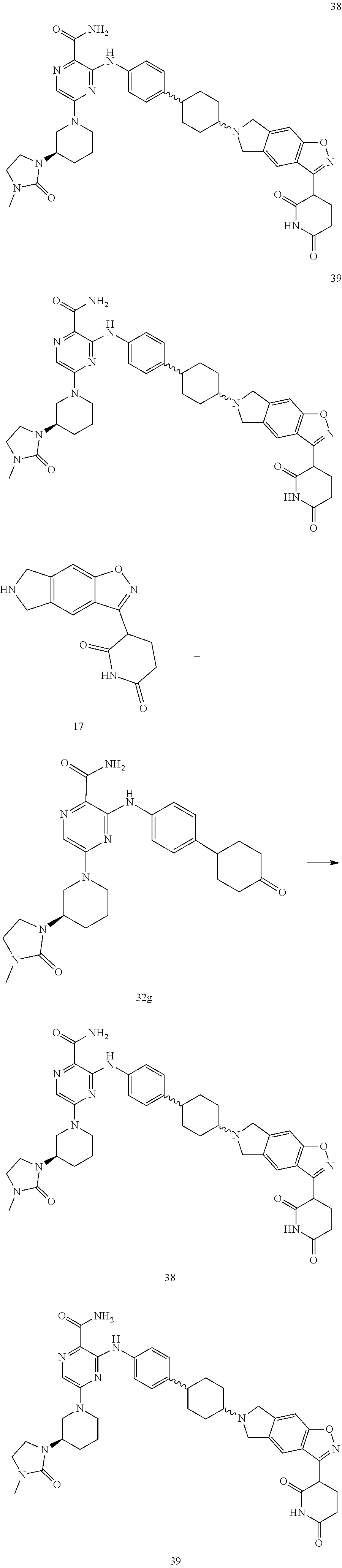

### Step 1: Preparation of Compounds 38 and 39

32g (130 mg), isopropanol (1 mL), 1,2-dichloroethane (5.00 mL), and 17 (122 mg) were added to a reaction flask in sequence, and sodium cyanoborohydride (49.9 mg) was added. The mixture was reacted at room temperature. After the reaction was completed, the reaction solution was mixed with silica gel and purified by column chromatography (MeOH:DCM=0:100-1:25) to give 38 (17 mg) and 39 (20 mg) in sequence. 38: MS(ESI, [M+H]+) m/z: 747.54.

¹H NMR (500 MHz, DMSO-d6) δ 11.16 (s, 1H), 11.09 (s, 1H), 7.73 (s, 1H), 7.68 (s, 1H), 7.64 (d, J=4.6 Hz, 2H), 7.45 (d, J=8.2 Hz, 2H), 7.31 (s, 1H), 7.11 (d, J=8.2 Hz, 2H), 4.61-4.53 (m, 1H), 4.38 (d, J=12.5 Hz, 1H), 4.24 (d, J=13.4 Hz, 1H), 3.95 (d, J=27.6 Hz, 4H), 3.62-3.52 (m, 1H), 3.30-3.22 (m, 2H), 3.07 (t, J=7.7 Hz, 2H), 2.94 (q, J=12.8, 12.3 Hz, 2H), 2.84-2.69 (m, 2H), 2.65-2.59 (m, 1H), 2.59-2.53 (m, 2H), 2.47 (d, J=3.1 Hz, 3H), 2.25-2.17 (m, 1H), 2.02 (d, J=13.6 Hz, 2H), 1.78 (tt, J=26.4, 12.2 Hz, 5H), 1.63 (t, J=13.3 Hz, 2H), 1.54 (d, J=11.7 Hz, 3H).

39: MS(ESI, [M+H]+) m/z: 747.53.

¹H NMR (500 MHz, DMSO-d6) δ 11.20 (s, 1H), 11.09 (s, 1H), 7.76 (d, J=2.9 Hz, 1H), 7.66 (d, J=2.5 Hz, 2H), 7.61 (s, 1H), 7.50 (d, J=8.1 Hz, 2H), 7.33 (d, J=2.8 Hz, 1H), 7.17 (d, J=8.4 Hz, 2H), 4.56 (dd, J=11.9, 5.0 Hz, 1H), 4.32 (dd, J=32.1, 12.1 Hz, 2H), 3.99 (d, J=26.3 Hz, 4H), 3.61 (tt, J=10.0, 3.9 Hz, 1H), 3.38-3.33 (m, 2H), 3.26 (dd, J=10.1, 7.1 Hz, 2H), 3.00 (dt, J=42.5, 12.1 Hz, 2H), 2.83-2.69 (m, 4H), 2.61 (dt, J=17.3, 4.4 Hz, 1H), 2.46 (d, J=11.6 Hz, 2H), 2.24-2.07 (m, 3H), 1.94-1.73 (m, 5H), 1.62-1.31 (m, 6H).

### Example 40: Synthesis of Compound 40

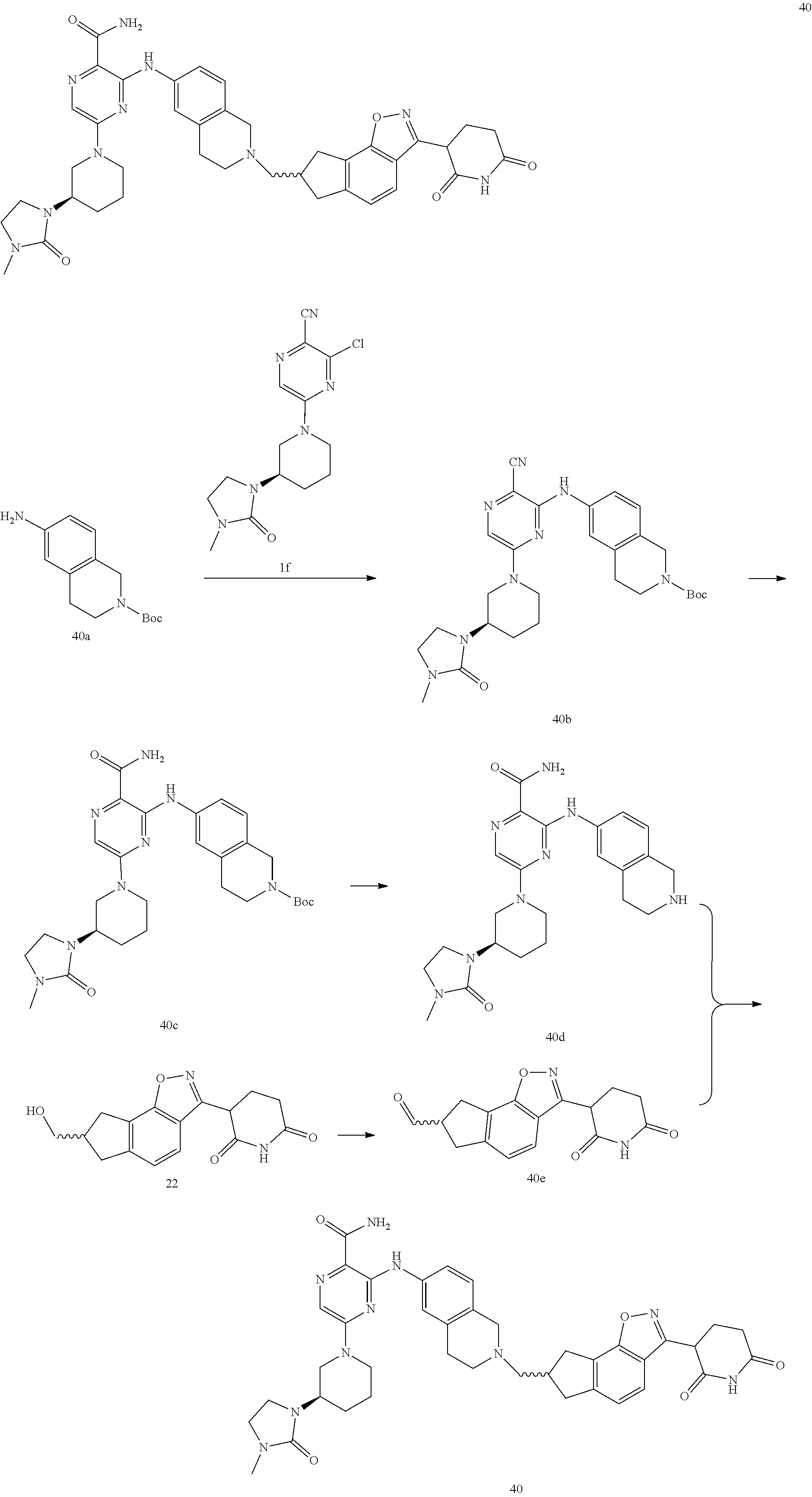

### Step 1: Preparation of Intermediate 0b

Intermediate 1f (1.5 g), intermediate 40a (1.161 g), BINAP (0.291 g), cesium carbonate (4.57 g), palladium acetate (0.105 g, 0.468 mmol), and 1,4-dioxane (20 mL) were added to a reaction flask in sequence, and the mixture was heated to 100° C. and reacted under N₂ atmosphere. After the reaction was completed, as confirmed by TLC, the reaction solution was diluted with 20 mL of dichloromethane and purified by silica gel column chromatography to give the target intermediate 0b (2.2 g).

MS(ESI, [M+H]⁺) m/z: 533.4.

¹H NMR (500 MHz, DMSO-d6) δ 8.98 (s, 1H), 7.83 (s, 1H), 7.43 (d, J=2.2 Hz, 1H), 7.29 (dd, J=8.3, 2.2 Hz, 1H), 7.05 (d, J=8.3 Hz, 1H), 4.44 (s, 2H), 4.38-4.19 (m, 2H), 3.57 (dt, J=11.6, 5.5 Hz, 1H), 3.52 (t, J=6.0 Hz, 2H), 3.28 (td, J=6.8, 3.7 Hz, 3H), 3.24-3.18 (m, 1H), 2.99 (s, 1H), 2.95-2.87 (m, 1H), 2.68 (s, 5H), 1.83-1.77 (i, 1H), 1.77-1.68 (m, 2H), 1.56-1.49 (m, 1H), 1.43 (s, 9H).

### Step 2: Preparation of Intermediate 40c

Intermediate 1b (2.2 g), DMSO (8 mL), methanol (20 mL), cesium carbonate (1.346 g), and water (8 mL) were added to a reaction flask in sequence, and 30 wt % hydrogen peroxide (1.405 g, 1.405 mL, 12.39 mmol) was added under an ice bath. The mixture was allowed to return to room temperature and react. After the reaction was completed, as confirmed by TLC, the reaction solution was poured into an icy saturated sodium sulfite solution to quench the reaction, and ethyl acetate was added for extraction. The organic phase was separated, washed with a saturated sodium chloride solution, dried over anhydrous sodium sulfate, and filtered, and the filtrate was concentrated by evaporation at reduced pressure to remove the solvent, thus giving the target intermediate 40c (2.1 g).

MS(ESI, [M+H]⁺) m/z: 551.40.

¹H NMR (500 MHz, DMSO-d6) δ 11.28 (s, 1H), 7.81-7.75 (m, 1H), 7.70-7.60 (m, 2H), 7.38 (s, 1H), 7.23-7.16 (m, 1H), 7.06 (d, J=8.5 Hz, 1H), 4.44 (s, 2H), 4.38 (d, J=12.5 Hz, 1H), 4.26 (d, J=13.3 Hz, 1H), 3.63 (dq, J=11.4, 5.9, 5.2 Hz, 1H), 3.53 (t, J=5.8 Hz, 2H), 3.35 (s, 1H), 3.33-3.20 (m, 3H), 3.01 (t, J=12.0 Hz, 1H), 2.96-2.87 (m, 1H), 2.69 (d, J=3.9 Hz, 5H), 1.77 (dddd, J=27.7, 23.7, 12.4, 3.9 Hz, 3H), 1.63-1.51 (m, 1H), 1.43 (s, 9H).

### Step 3: Preparation of Intermediate 40d

Intermediate 40c (2.1 g) and TFA (8.70 g, 5.88 mL, 76 mmol) were added to a reaction flask in sequence, and the mixture was reacted at room temperature. After the reaction was completed, as confirmed by TLC, the reaction solution was concentrated by evaporation at reduced pressure to remove the solvent, 20 mL of dichloromethane was added, and the mixture was subjected to rotary evaporation. The process was repeated 2 times. The residue was completely dissolved in 10 mL of dichloromethane, then the solution was added dropwise to 100 mL of an icy 1 M sodium hydroxide solution, and 100 mL of dichloromethane was added for extraction. The organic phase was separated, and the aqueous phase was extracted twice with 50 mL of dichloromethane. The organic phases were combined, washed with saturated brine, dried over anhydrous sodium sulfate, and filtered, and the filtrate was concentrated by evaporation at reduced pressure to remove the solvent, thus giving the target intermediate 40d (1.7 g).

MS(ESI, [M+H]⁺) m/z: 451.45.

¹H NMR (500 MHz, DMSO-d6) δ 11.25 (d, J=19.0 Hz, 1H), 7.77 (dd, J=5.6, 2.8 Hz, 1H), 7.67 (d, J=6.0 Hz, 1H), 7.57 (dd, J=7.5, 2.2 Hz, 1H), 7.36 (dq, J=6.2, 3.1 Hz, 1H), 7.16 (ddd, J=34.2, 8.2, 2.3 Hz, 1H), 6.98 (dd, J=11.8, 8.3 Hz, 1H), 4.37 (d, J=12.5 Hz, 1H), 4.28 (d, J=13.3 Hz, 1H), 3.94 (s, 1H), 3.61 (tdd, J=15.2, 9.5, 5.2 Hz, 2H), 3.37-3.18 (m, 5H), 3.10-3.04 (m, 2H), 2.94 (td, J=12.8, 2.8 Hz, 1H), 2.75 (d, J=8.0 Hz, 2H), 2.68 (s, 2H), 2.64 (s, 1H), 2.51 (s, 1H), 1.87-1.70 (m, 3H), 1.64-1.50 (m, 1H).

### Step 4: Preparation of Intermediate 40e

Intermediate 22 (50 mg), Dess-Martin periodinane (141 mg, 0.333 mmol), dichloromethane (5 mL), and DMF (1 mL) were added to a reaction flask in sequence, and the mixture was reacted at room temperature. After the reaction was completed, as confirmed by TLC, the reaction solution was extracted three times with 50 mL of water and 20 mL of ethyl acetate. The organic phases were combined, washed with 50 mL of a saturated sodium chloride solution, dried over anhydrous sodium sulfate, and filtered, and the filtrate was concentrated by evaporation at reduced pressure to remove the solvent, thus giving the target intermediate 40e (50 mg).

MS(ESI, [M+H]⁺) m/z: 298.31.

### Step 5: Preparation of Compound 40

Intermediate 40e (50 mg), 1,2-dichloroethane (6 mL), isopropanol (2 mL), intermediate 40d (83 mg), acetic acid (5.00 mg, 0.083 mmol), and sodium cyanoborohydride (20.93 mg, 0.333 mmol) were added to a reaction flask in sequence, and the mixture was stirred and reacted at room temperature. After the reaction was completed, as confirmed by TLC, 2 mL of a saturated sodium bicarbonate solution was added to the reaction solution to neutralize acetic acid, and then 50 mL of dichloromethane and 100 mL were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 40 (26 mg).

MS(ESI, [M+H]⁺) m/z: 733.42.

¹H NMR (500 MHz, DMSO-d6) δ 11.23 (s, 1H), 11.08 (d, J=1.8 Hz, 1H), 7.77 (d, J=2.8 Hz, 1H), 7.67 (s, 1H), 7.61 (d, J=8.1 Hz, 1H), 7.58 (s, 1H), 7.35 (d, J=2.9 Hz, 1H), 7.27 (d, J=8.1 Hz, 1H), 7.17-7.10 (m, 1H), 6.97 (d, J=8.3 Hz, 1H), 4.57 (ddd, J=11.9, 4.9, 1.7 Hz, 1H), 4.38 (d, J=12.5 Hz, 1H), 4.32-4.24 (m, 1H), 3.66-3.53 (m, 3H), 3.35 (s, 1H), 3.31-3.13 (m, 5H), 3.10-2.83 (m, 6H), 2.76 (dt, J=17.2, 5.8 Hz, 3H), 2.67 (s, 5H), 2.63 (t, J=3.6 Hz, 1H), 2.59 (s, 1H), 2.46 (d, J=5.8 Hz, 1H), 2.18 (dt, J=13.1, 4.5 Hz, 1H), 1.78 (dtd, J=22.0, 11.4, 6.5 Hz, 3H), 1.57 (q, J=12.3, 11.5 Hz, 1H).

### Example 41: Synthesis of Compound 41

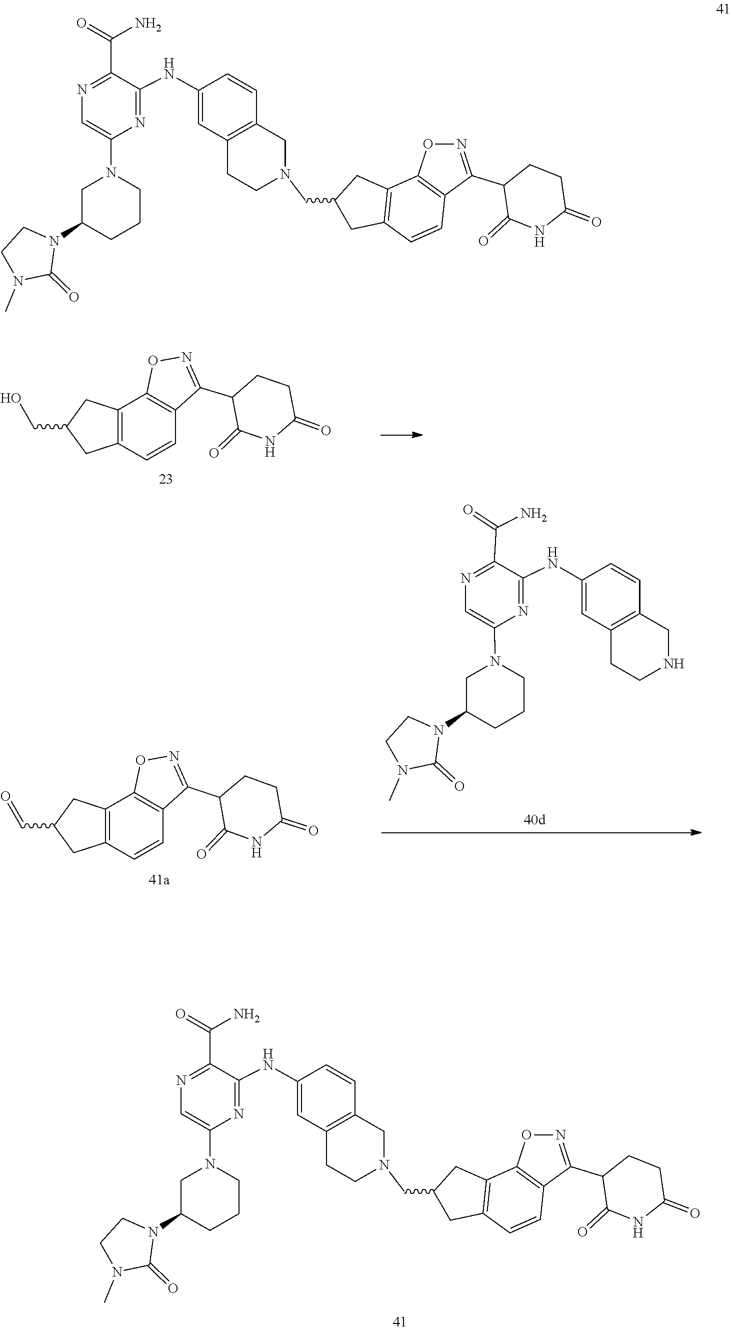 [SMILES: CN1CCN([C@@H]2CCCN(c3cnc(C(N)=O)c(Nc4ccc5c(c4)CCN(CC4Cc6ccc7c(C8CCC(=O)NC8=O)noc7c6C4)C5)n3)C2)C1=O ; O=C1CCC(c2noc3c4c(ccc23)CC(CO)C4)C(=O)N1 ; O=CC1Cc2ccc3c(C4CCC(=O)NC4=O)noc3c2C1 ; CN1CCN([C@@H]2CCCN(c3cnc(C(N)=O)c(Nc4ccc5c(c4)CCNC5)n3)C2)C1=O ; CN1CCN([C@@H]2CCCN(c3cnc(C(N)=O)c(Nc4ccc5c(c4)CCN(CC4Cc6ccc7c(C8CCC(=O)NC8=O)noc7c6C4)C5)n3)C2)C1=O]

### Step 1: Preparation of Intermediate 41a

Intermediate 23 (50 mg), Dess-Martin periodinane (141 mg, 0.333 mmol), dichloromethane (5 mL), and DMF (1 mL) were added to a reaction flask in sequence, and the mixture was reacted at room temperature. After the reaction was completed, as confirmed by TLC, the reaction solution was extracted three times with 50 mL of water and 20 mL of ethyl acetate. The organic phases were combined, washed with a saturated sodium chloride solution, dried over anhydrous sodium sulfate, and filtered, and the filtrate was concentrated by evaporation at reduced pressure to remove the solvent, thus giving the target intermediate 41a (50 mg).

MS(ESI, [M+H]⁺) m/z: 298.29.

### Step 2: Preparation of Compound 41

Intermediate 41a (50 mg), 1,2-dichloroethane (6 mL), isopropanol (2 mL), intermediate 40d (83 mg), acetic acid (5.00 mg, 0.083 mmol), and sodium cyanoborohydride (20.93 mg, 0.333 mmol) were added to a reaction flask in sequence, and the mixture was stirred and reacted at room temperature. After the reaction was completed, as confirmed by TLC, 2 mL of a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 41 (26 mg).

MS(ESI, [M+H]⁺) m/z: 733.40.

¹H NMR (500 MHz, DMSO-d6) δ 11.23 (s, 1H), 11.09 (s, 1H), 7.79-7.74 (m, 1H), 7.67 (s, 1H), 7.63-7.56 (m, 2H), 7.38-7.33 (m, 1H), 7.27 (d, J=8.1 Hz, 1H), 7.14 (d, J=8.2 Hz, 1H), 6.97 (d, J=8.3 Hz, 1H), 4.57 (dd, J=12.0, 4.9 Hz, 1H), 4.38 (d, J=12.5 Hz, 1H), 4.32-4.25 (m, 1H), 3.67-3.52 (m, 3H), 3.30-3.14 (m, 5H), 3.12-2.84 (m, 6H), 2.83-2.68 (m, 5H), 2.67 (s, 3H), 2.64-2.61 (m, 1H), 2.59 (s, 1H), 2.47 (d, J=14.4 Hz, 1H), 2.18 (dt, J=13.4, 4.6 Hz, 1H), 1.86-1.72 (m, 3H), 1.57 (d, J=12.7 Hz, 1H).

### Example 42: Synthesis of Compound 42

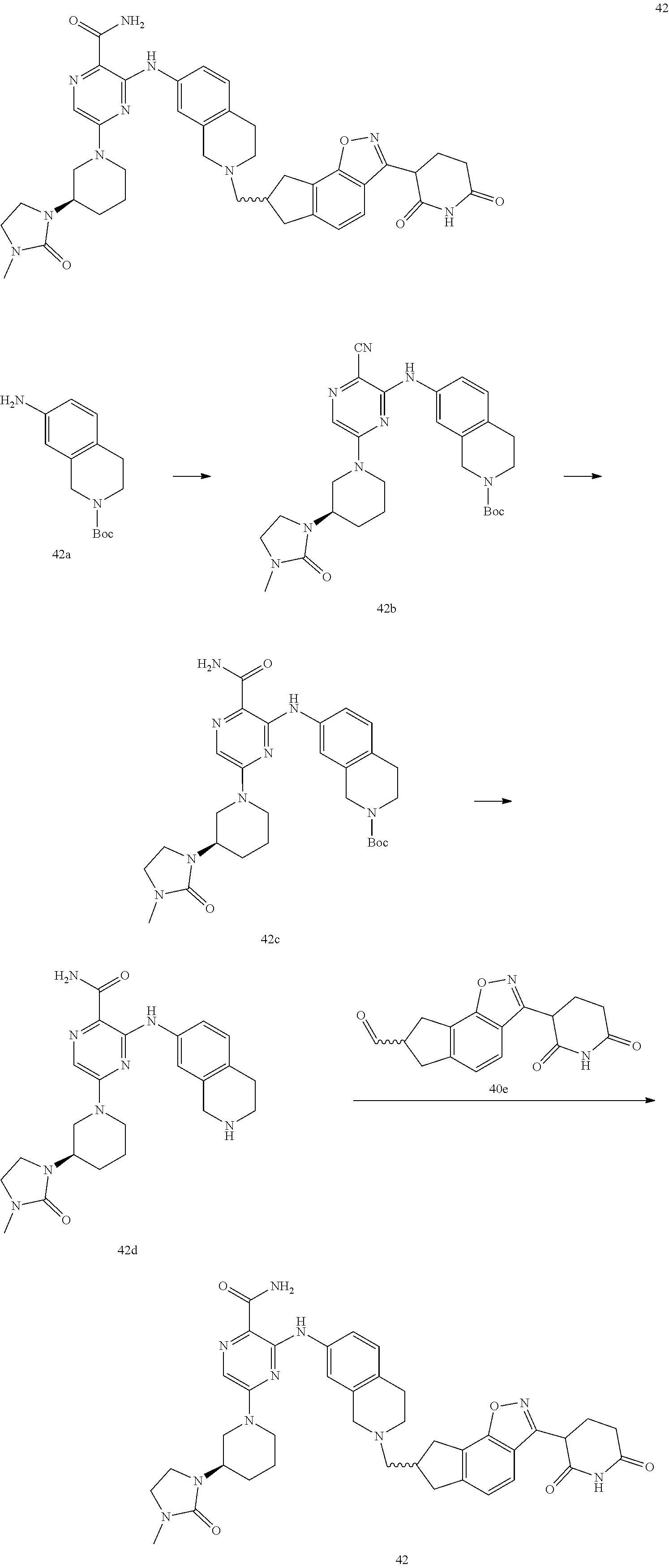

### Step 1: Preparation of Intermediate 42b

42a (1.16 g), 1f (1.50 g), BINAP (0.29 g), cesium carbonate (4.57 g), palladium acetate (0.10 g), and 1,4-dioxane (50 mL) were added to a reaction flask in sequence, and the mixture was purged three times with nitrogen, heated to 100° C., and reacted. After the reaction was completed, as confirmed by TLC, the reaction solution was cooled to room temperature, and 80 L of ethyl acetate and 140 mL of water were added to the residue. The organic phase was separated, washed with a saturated sodium chloride solution, dried over anhydrous sodium sulfate, filtered, and concentrated. The concentrate was separated and purified by silica gel column chromatography to give intermediate 42b (2.2 g).

MS(ESI, [M+H]⁺) m/z: 533.5.

### Step 2: Preparation of Intermediate 42c

Intermediate 42b (2.2 g), cesium carbonate (1.34 g), water (10 mL), DMSO (30 mL), and methanol (10 mL) were added to a reaction flask in sequence. The reaction solution was cooled to 0° C., and 30 wt % hydrogen peroxide (1.40 g) was added dropwise. After the dropwise addition was completed, the mixture was allowed to react at room temperature. After the reaction was completed, as confirmed by TLC, 100 mL of water was added to the reaction solution, and then a saturated aqueous sodium sulfite solution was added to neutralize hydrogen peroxide. The mixture was stirred for 10 min and filtered, and the filter cake was collected and dried to give intermediate 42c (1.9 g).

MS(ESI, [M+H]⁺) m/z: 551.4.

### Step 3: Preparation of Intermediate 42d

Intermediate 42c (0.30 g), dichloromethane (20 mL), and trifluoroacetic acid (1 mL) were added to a reaction flask in sequence, and the mixture was reacted at room temperature. After the reaction was completed, as confirmed by TLC, 50 mL of water was added to the reaction solution, the pH was adjusted to about 8 with a saturated aqueous sodium bicarbonate solution, and the product was extracted with DCM:MEOH=10:1. The organic phase was separated, dried over anhydrous sodium sulfate, filtered, and concentrated to give intermediate 42d (0.20 g).

MS(ESI, [M+H]⁺) m/z: 451.2.

### Step 4: Preparation of Compound 42

Intermediate 42d (76 mg), 40e (50 mg), dichloroethane/isopropanol (5:1, 20 mL), glacial acetic acid (10 mg), and sodium cyanoborohydride (21 mg) were added to a reaction flask in sequence, and the mixture was reacted at room temperature. After the reaction was completed, as confirmed by TLC, dichloromethane and water were added to the reaction solution. The organic phase was separated, washed with a saturated sodium chloride solution, dried over anhydrous sodium sulfate, filtered, and concentrated. The concentrate was separated and purified by silica gel column chromatography to give compound 42 (20 mg).

MS(ESI, [M+H]⁺) m/z: 733.5.

1H NMR (500 MHz, DMSO-d6) δ 11.22 (s, 1H), 11.08 (d, J=1.8 Hz, 1H), 7.76 (d, J=2.8 Hz, 1H), 7.66 (s, 1H), 7.62 (d, J=8.0 Hz, 1H), 7.44 (d, J=2.2 Hz, 1H), 7.34 (d, J=2.8 Hz, 1H), 7.27 (d, J=8.1 Hz, 1H), 7.21 (d, J=8.6 Hz, 1H), 7.03 (d, J=8.3 Hz, 1H), 4.57 (ddd, J=11.9, 5.0, 2.5 Hz, 1H), 4.30 (dd, J=31.6, 12.6 Hz, 2H), 3.63-3.48 (m, 3H), 3.31-3.22 (m, 3H), 3.19 (dd, J=9.0, 6.6 Hz, 2H), 3.12 (d, J=8.4 Hz, 1H), 3.06 (t, J=11.8 Hz, 1H), 2.99 (d, J=6.9 Hz, 1H), 2.97-2.91 (m, 2H), 2.88 (dd, J=16.5, 4.7 Hz, 1H), 2.81-2.72 (m, 4H), 2.68 (d, J=5.1 Hz, 1H), 2.65-2.61 (m, 1H), 2.59 (d, J=1.2 Hz, 3H), 2.18 (dq, J=8.7, 4.2 Hz, 1H), 1.83-1.73 (m, 3H), 1.61-1.52 (m, 1H).

### Example 43: Synthesis of Compound 43

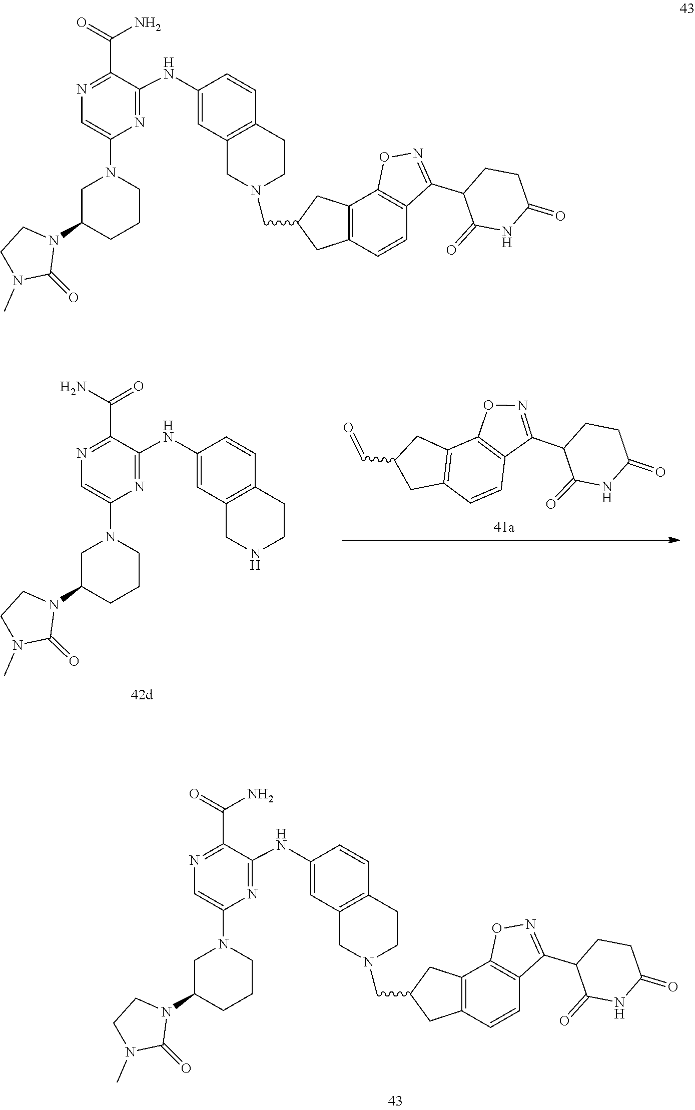 [SMILES: CN1CCN([C@@H]2CCCN(c3cnc(C(N)=O)c(Nc4ccc5c(c4)CN(CC4Cc6ccc7c(C8CCC(=O)NC8=O)noc7c6C4)CC5)n3)C2)C1=O ; CN1CCN([C@@H]2CCCN(c3cnc(C(N)=O)c(Nc4ccc5c(c4)CNCC5)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc5c(c4)CN(CC4Cc6ccc7c(C8CCC(=O)NC8=O)noc7c6C4)CC5)n3)C2)C1=O]

### Step 1: Preparation of Compound 43

Intermediate 42d (76 mg), 41a (50 mg), dichloroethane/isopropanol (5:1, 20 mL), glacial acetic acid (10 mg), and sodium cyanoborohydride (21 mg) were added to a reaction flask in sequence, and the mixture was reacted at room temperature. After the reaction was completed, as confirmed by TLC, dichloromethane and water were added to the reaction solution. The organic phase was separated, washed with a saturated sodium chloride solution, dried over anhydrous sodium sulfate, filtered, and concentrated. The concentrate was separated and purified by silica gel column chromatography to give compound 43 (24 mg).

MS(ESI, [M+H]⁺) m/z: 733.5.

1H NMR (500 MHz, DMSO-d6) δ 11.22 (s, 1H), 11.08 (s, 1H), 7.76 (d, J=2.8 Hz, 1H), 7.66 (s, 1H), 7.62 (d, J=8.0 Hz, 1H), 7.44 (d, J=2.2 Hz, 1H), 7.35 (d, J=2.8 Hz, 1H), 7.27 (d, J=8.1 Hz, 1H), 7.21 (d, J=8.3 Hz, 1H), 7.03 (d, J=8.3 Hz, 1H), 4.57 (ddd, J=11.9, 5.0, 2.6 Hz, 1H), 4.30 (dd, J=31.1, 12.8 Hz, 2H), 3.64-3.55 (m, 2H), 3.51 (d, J=15.2 Hz, 1H), 3.31-3.25 (m, 2H), 3.25-3.21 (m, 1H), 3.21-3.14 (m, 2H), 3.13-3.02 (m, 2H), 2.99 (d, J=6.5 Hz, 1H), 2.96 (s, 1H), 2.94-2.85 (m, 2H), 2.83-2.71 (m, 4H), 2.67 (t, J=5.7 Hz, 1H), 2.62 (t, J=3.8 Hz, 1H), 2.59 (d, J=2.8 Hz, 3H), 2.18 (dq, J=8.6, 4.5 Hz, 1H), 1.83-1.71 (m, 3H), 1.56 (d, J=14.2 Hz, 2H).

### Example 44: Synthesis of Compound 44

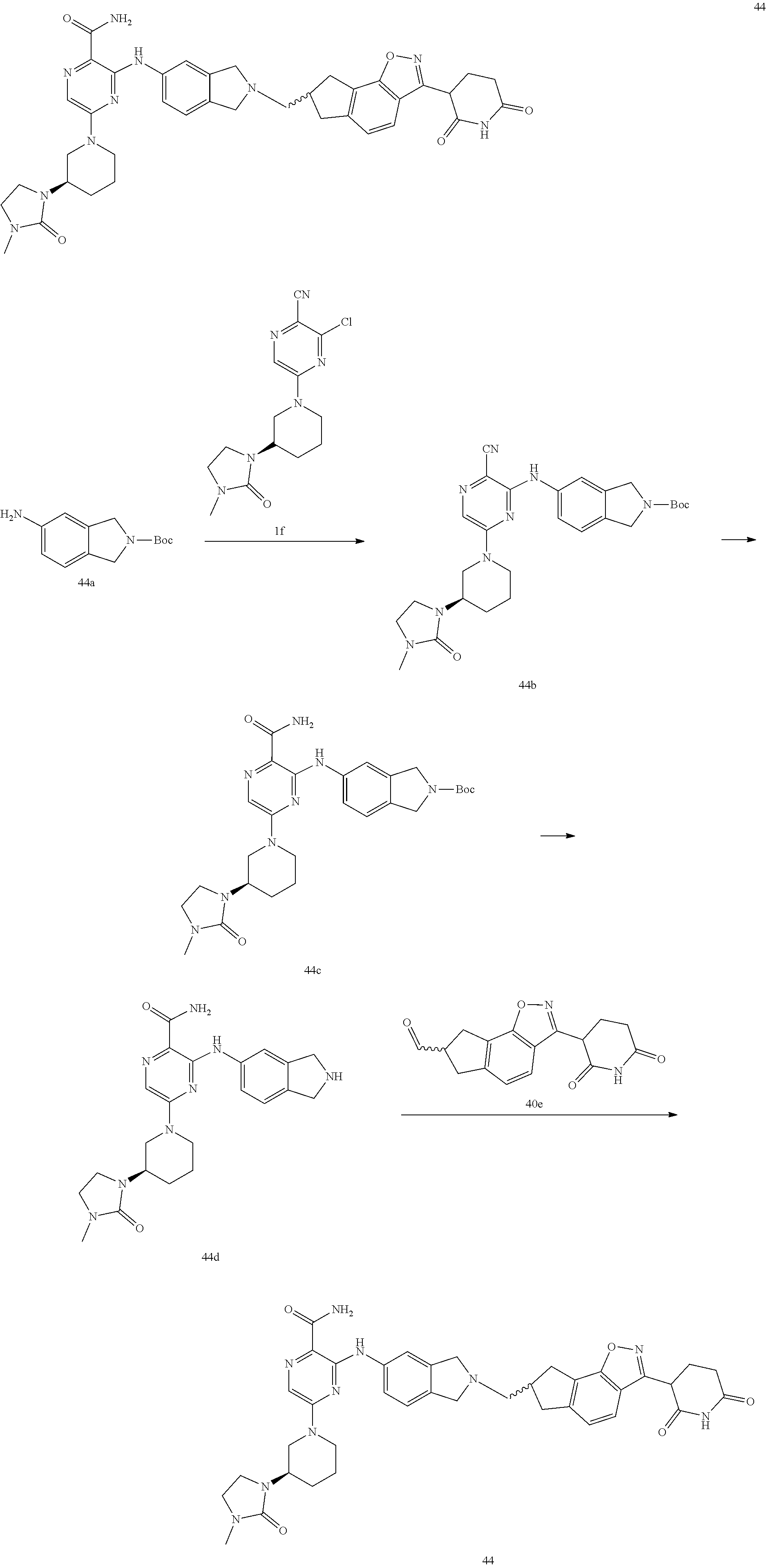

### Step 1: Preparation of Intermediate 44b

Intermediate 1f (1.5 g), intermediate 44a (1.315 g), BINAP (0.291 g, 0.468 mmol), cesium carbonate (4.57 g), palladium acetate (0.105 g, 0.468 mmol), and 1,4-dioxane (20 mL) were added to a reaction flask in sequence, and the mixture was heated to 100° C. and reacted under N₂ atmosphere. After the reaction was completed, as confirmed by TLC, the reaction solution was diluted with 20 mL of dichloromethane and purified by silica gel column chromatography to give the target intermediate 44b (1.99 g).

MS(ESI, [M+H]⁺) m/z: 519.34.

### Step 2: Preparation of Intermediate 44c

Intermediate 44b (1.99 g), DMSO (8 mL), methanol (20 mL), cesium carbonate (1.250 g), and water (8 mL) were added to a reaction flask in sequence, and 30 wt % hydrogen peroxide (1.305 g, 1.305 mL, 11.51 mmol) was added under an ice bath. The mixture was allowed to return to room temperature and react. After the reaction was completed, as confirmed by TLC, the reaction solution was poured into 200 mL of an icy saturated sodium sulfite solution to quench the reaction, and 200 mL of ethyl acetate was added for extraction. The organic phase was separated, and the aqueous phase was extracted twice with 50 mL of ethyl acetate. The organic phases were combined, washed with a saturated sodium chloride solution, dried over anhydrous sodium sulfate, and filtered, and the filtrate was concentrated by evaporation at reduced pressure to remove the solvent, thus giving the target intermediate 44c (1.8 g).

MS(ESI, [M+H]⁺) m/z: 537.40.

¹H NMR (500 MHz, DMSO-d6) δ 11.35 (d, J=3.5 Hz, 1H), 7.79 (d, J=2.6 Hz, 1H1), 7.69 (d, J=2.5 Hz, 1H), 7.36 (dd, J=12.2, 2.6 Hz, 2H), 7.23 (q, J=8.3 Hz, 2H), 4.53 (q, J=11.5, 10.6 Hz, 4H), 4.39 (d, J=14.0 Hz, 1H), 4.25 (t, J=13.3 Hz, 1H), 3.67-3.57 (m, 1H), 3.34 (d, J=5.9 Hz, 1H), 3.28 (h, J=5.7 Hz, 2H), 3.04 (td, J=11.7, 6.7 Hz, 1H), 2.96 (td, J=12.9, 2.5 Hz, 1H), 2.70 (d, J=10.8 Hz, 3H), 1.78 (h, J=12.7, 11.1 Hz, 3H), 1.57 (dt, J=15.8, 4.9 Hz, 1H), 1.46 (d, J=4.7 Hz, 9H).

### Step 3: Preparation of Intermediate 44d

Intermediate 44c (1.8 g) and TFA (7.65 g, 5.17 mL, 67.1 mmol) were added to a reaction flask in sequence, and the mixture was reacted at room temperature. After the reaction was completed, as confirmed by TLC, the reaction solution was concentrated by evaporation at reduced pressure to remove the solvent. The residue was completely dissolved in 10 mL of dichloromethane, then the solution was added dropwise to 100 mL of an icy 1 M sodium hydroxide solution, and 100 mL of dichloromethane was added for extraction. The organic phase was separated, and the aqueous phase was extracted twice with 50 mL of dichloromethane. The organic phases were combined, washed with saturated brine, dried over anhydrous sodium sulfate, and filtered, and the filtrate was concentrated by evaporation at reduced pressure to remove the solvent, thus giving the target intermediate 44d (1.36 g).

MS(ESI, [M+H]⁺) m/z: 437.30.

¹H NMR (500 MHz, DMSO-d6) δ 11.28 (s, 1H), 7.77 (d, J=2.8 Hz, 1H), 7.67 (s, 1H), 7.63 (d, J=2.0 Hz, 1H), 7.35 (d, J=3.1 Hz, 1H), 7.23 (dd, J=8.0, 2.0 Hz, 1H), 7.15 (d, J=8.0 Hz, 1H), 4.36 (d, J=12.8 Hz, 1H), 4.27 (d, J=13.4 Hz, 1H), 4.02 (s, 4H), 3.60 (dtd, J=14.9, 10.6, 5.2 Hz, 1H), 3.30-3.20 (m, 3H), 3.04 (t, J=11.8 Hz, 1H), 2.94 (td, J=12.9, 2.6 Hz, 1H), 2.69 (s, 3H), 1.83-1.73 (m, 3H), 1.60-1.52 (m, 1H).

### Step 4: Preparation of Compound 44

Intermediate 40e (50 mg), 1,2-dichloroethane (6 mL), isopropanol (2 mL), intermediate 44d (72.7 mg), acetic acid (5.00 mg, 0.083 mmol), and sodium cyanoborohydride (20.93 mg, 0.333 mmol) were added to a reaction flask in sequence, and the mixture was stirred and reacted at room temperature. After the reaction was completed, as confirmed by TLC, 2 mL of a saturated sodium bicarbonate solution was added to the reaction solution, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 44 (40 mg).

MS(ESI, [M+H]⁺) m/z: 719.50.

¹H NMR (500 MHz, DMSO-d6) δ 11.30 (d, J=3.0 Hz, 1H), 11.09 (s, 1H), 7.77 (d, J=2.9 Hz, 1H), 7.67 (d, J=4.0 Hz, 2H), 7.62 (d, J=8.0 Hz, 1H), 7.36 (d, J=2.9 Hz, 1H), 7.28 (d, J=8.1 Hz, 1H), 7.22 (d, J=8.3 Hz, 1H), 7.15 (d, J=8.1 Hz, 1H), 4.57 (dd, J=11.9, 4.9 Hz, 1H), 4.37 (d, J=12.7 Hz, 1H), 4.26 (d, J=13.3 Hz, 1H), 3.87 (s, 4H), 3.61 (tt, J=10.3, 4.7 Hz, 1H), 3.29 (d, J=9.6 Hz, 2H), 3.22 (t, J=7.4 Hz, 3H), 3.05-2.86 (m, 5H), 2.77 (ddt, J=18.5, 12.1, 5.9 Hz, 3H), 2.64-2.58 (m, 4H), 2.46 (d, J=4.3 Hz, 1H), 2.23-2.15 (m, 1H), 1.86-1.69 (m, 3H), 1.61-1.51 (m, 1H), 1.24 (d, J=6.3 Hz, 3H).

### Example 45: Synthesis of Compound 45

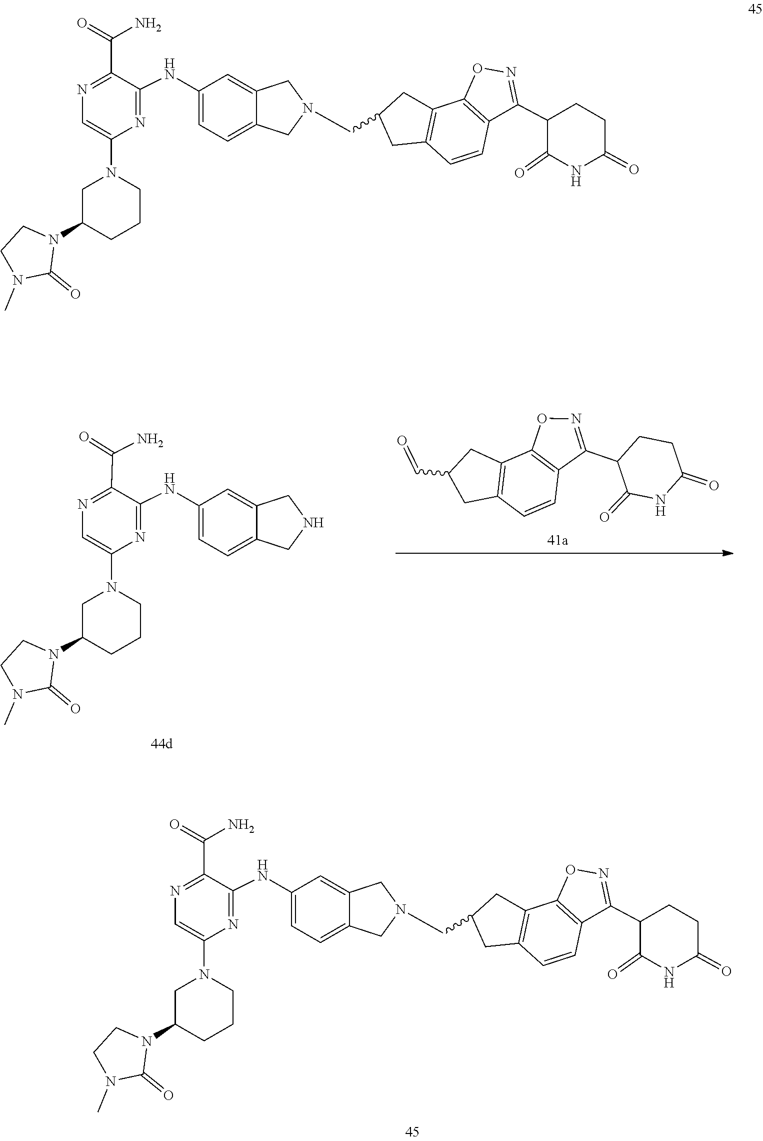 [SMILES: CN1CCN([C@@H]2CCCN(c3cnc(C(N)=O)c(Nc4ccc5c(c4)CN(CC4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CNC5)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc5c(c4)CN(CC4Cc6ccc7c(C8CCC(=O)NC8=O)noc7c6C4)C5)n3)C2)C1=O]

Intermediate 41a (50 mg), 1,2-dichloroethane (6 mL), isopropanol (2 mL), intermediate 44d (72.7 mg), acetic acid (5.00 mg, 0.083 mmol), and sodium cyanoborohydride (20.93 mg, 0.333 mmol) were added to a reaction flask in sequence, and the mixture was stirred and reacted at room temperature. After the reaction was completed, as confirmed by TLC, 2 mL of a saturated sodium bicarbonate solution was added to the reaction solution, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 45 (60 mg).

MS(ESI, [M+H]⁺) m/z: 719.40.

¹H NMR (500 MHz, DMSO-d6) δ 11.30 (d, J=2.9 Hz, 1H), 11.09 (s, 1H), 7.77 (d, J=2.8 Hz, 1H), 7.67 (d, J=4.0 Hz, 2H), 7.62 (d, J=8.0 Hz, 1H), 7.36 (d, J=2.9 Hz, 1H), 7.28 (d, J=8.1 Hz, 1H), 7.22 (d, J=8.3 Hz, 1H), 7.15 (d, J=8.1 Hz, 1H), 4.57 (dd, J=11.9, 5.0 Hz, 1H), 4.37 (d, J=12.6 Hz, 1H), 4.26 (d, J=13.3 Hz, 1H), 3.87 (s, 4H), 3.61 (dq, J=11.0, 5.5, 5.0 Hz, 1H), 3.31-3.25 (m, 2H), 3.25-3.16 (m, 3H), 3.12-2.82 (m, 6H), 2.81-2.70 (m, 3H), 2.65-2.58 (m, 4H), 2.49-2.44 (m, 1H), 2.19 (dp, J=12.9, 4.5 Hz, 1H), 1.85-1.71 (m, 3H), 1.56 (qd, J=13.0, 12.4, 6.2 Hz, 1H).

### Example 46: Synthesis of Compound 46

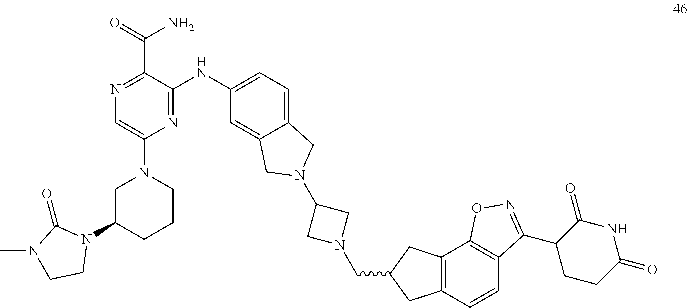 [SMILES: CN1CCN([C@@H]2CCCN(c3cnc(C(N)=O)c(Nc4ccc5c(c4)CN(C4CN(CC6Cc7ccc8c(C9CCC(=O)NC9=O)noc8c7C6)C4)C5)n3)C2)C1=O]

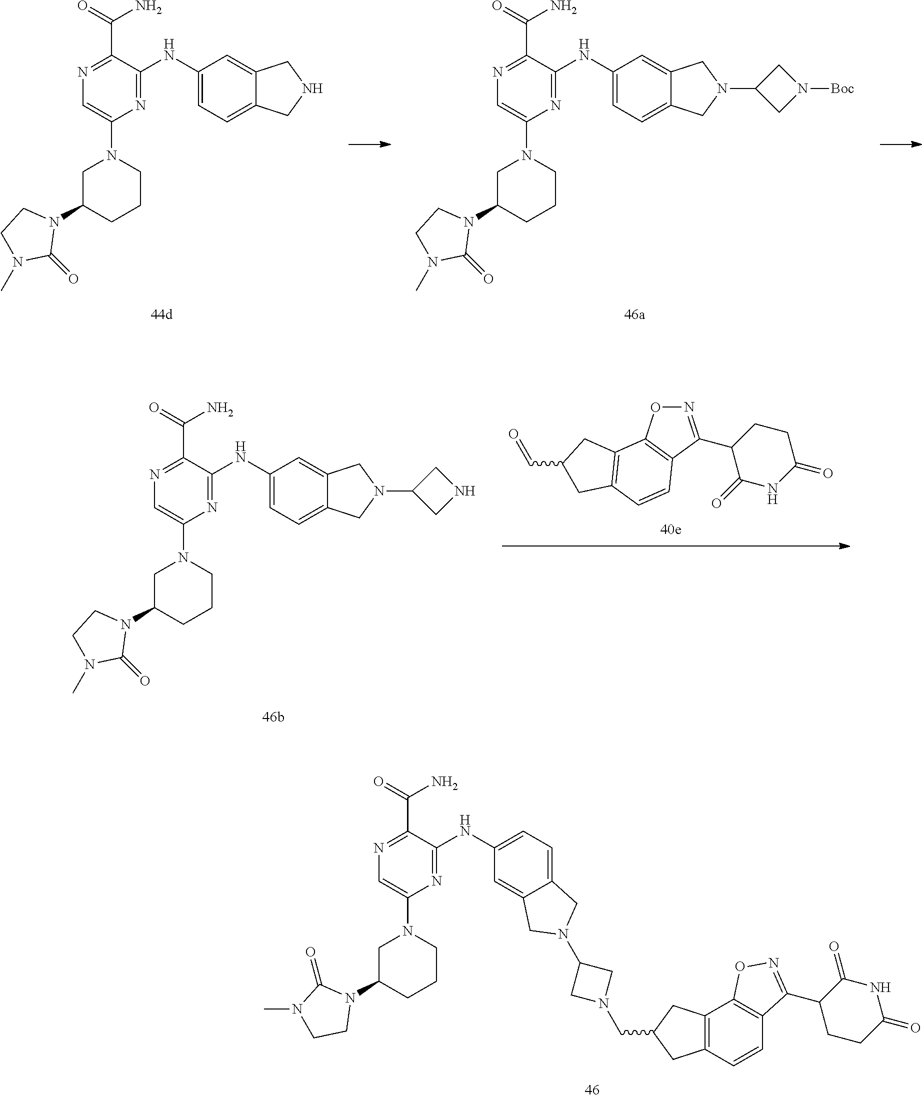 [SMILES: CN1CCN([C@@H]2CCCN(c3cnc(C(N)=O)c(Nc4ccc5c(c4)CNC5)n3)C2)C1=O ; CN1CCN([C@@H]2CCCN(c3cnc(C(N)=O)c(Nc4ccc5c(c4)CN(C4CN(C(=O)OC(C)(C)C)C4)C5)n3)C2)C1=O ; CN1CCN([C@@H]2CCCN(c3cnc(C(N)=O)c(Nc4ccc5c(c4)CN(C4CNC4)C5)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc5c(c4)CN(C4CN(CC6Cc7ccc8c(C9CCC(=O)NC9=O)noc8c7C6)C4)C5)n3)C2)C1=O]

### Step 1: Preparation of Intermediate 46a

Intermediate 44d (700 mg), 1-Boc-3-azetidinone (549 mg), 1,2-dichloroethane (3 mL), isopropanol (1 mL), acetic acid (48.1 mg, 0.802 mmol), and sodium cyanoborohydride (202 mg, 3.21 mmol) were added to a reaction flask in sequence, and the mixture was stirred and reacted at room temperature. After the reaction was completed, as confirmed by TLC, 20 mL of a saturated sodium bicarbonate solution was added to the reaction solution, and then 100 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give the target intermediate 46a (601 mg).

MS(ESI, [M+H]⁺) m/z: 592.51.

### Step 2: Preparation of Intermediate 46b

Intermediate 46a (601 mg) and TFA (2316 mg, 1565 μL, 20.31 mmol) were added to a reaction flask in sequence, and the mixture was reacted at room temperature for 1 h. After the reaction was completed, the reaction solution was concentrated by evaporation at reduced pressure to remove the solvent. The residue was completely dissolved in 10 mL of dichloromethane, then the solution was added dropwise to 100 mL of an icy 1 M sodium hydroxide solution, and 100 mL of dichloromethane was added for extraction. The organic phase was separated, and the aqueous phase was extracted twice with 50 mL of dichloromethane. The organic phases were combined, washed with saturated brine, dried over anhydrous sodium sulfate, and filtered, and the filtrate was concentrated by evaporation at reduced pressure to remove the solvent, thus giving the target intermediate 46b (434 mg).

MS(ESI, [M+H]⁺) m/z: 492.30.

¹H NMR (500 MHz, DMSO-d6) δ 11.30 (d, J=6.8 Hz, 1H), 7.77 (dd, J=5.3, 2.8 Hz, 1H), 7.70-7.61 (m, 2H), 7.35 (t, J=2.9 Hz, 1H), 7.29-7.18 (m, 1H), 7.14 (dd, J=11.9, 8.1 Hz, 1H), 4.35 (d, J=12.6 Hz, 1H), 4.27 (d, J=11.8 Hz, 1H), 3.87-3.69 (m, 8H), 3.63-3.58 (m, 1H), 3.36-3.29 (m, 3H), 3.28-3.23 (m, 2H), 3.03 (qd, J=10.2, 8.9, 5.7 Hz, 2H), 2.99-2.92 (m, 1H), 2.68 (s, 3H), 1.84-1.74 (m, 3H), 1.60-1.52 (m, 1H).

### Step 3: Preparation of Compound 46

Intermediate 40e (50 mg), 1,2-dichloroethane (6 mL), isopropanol (2 mL), intermediate 46b (83 mg), acetic acid (5.00 mg, 0.083 mmol), and sodium cyanoborohydride (20.93 mg, 0.333 mmol) were added to a reaction flask in sequence, and the mixture was stirred and reacted at room temperature. After the reaction was completed, as confirmed by TLC, 2 mL of a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 46 (20 mg).

MS(ESI, [M+H]⁺) m/z: 774.47.

¹H NMR (500 MHz, DMSO-d6) δ 11.31 (s, 1H), 11.08 (s, 1H), 7.80-7.74 (m, 1H), 7.66 (d, J=8.8 Hz, 2H), 7.61 (d, J=8.0 Hz, 1H), 7.39-7.33 (m, 1H), 7.25 (d, J=8.1 Hz, 1H), 7.21 (d, J=8.2 Hz, 1H), 7.14 (d, J=8.1 Hz, 1H), 4.56 (dd, J=11.8, 5.0 Hz, 1H), 4.37 (d, J=12.4 Hz, 1H), 4.26 (d, J=13.4 Hz, 1H), 3.83-3.73 (m, 4H), 3.61 (dt, J=10.9, 5.7 Hz, 1H), 3.49 (d, J=15.5 Hz, 3H), 3.28-3.11 (m, 5H), 2.99 (dt, J=42.1, 11.7 Hz, 4H), 2.90-2.72 (m, 4H), 2.69 (s, 3H), 2.62 (q, J=4.2 Hz, 2H), 2.59 (d, J=4.0 Hz, 1H), 2.49-2.43 (m, 1H), 2.18 (dq, J=13.7, 4.8 Hz, 1H), 1.84-1.74 (m, 3H), 1.56 (d, J=12.8 Hz, 1H).

### Example 47: Synthesis of Compound 47

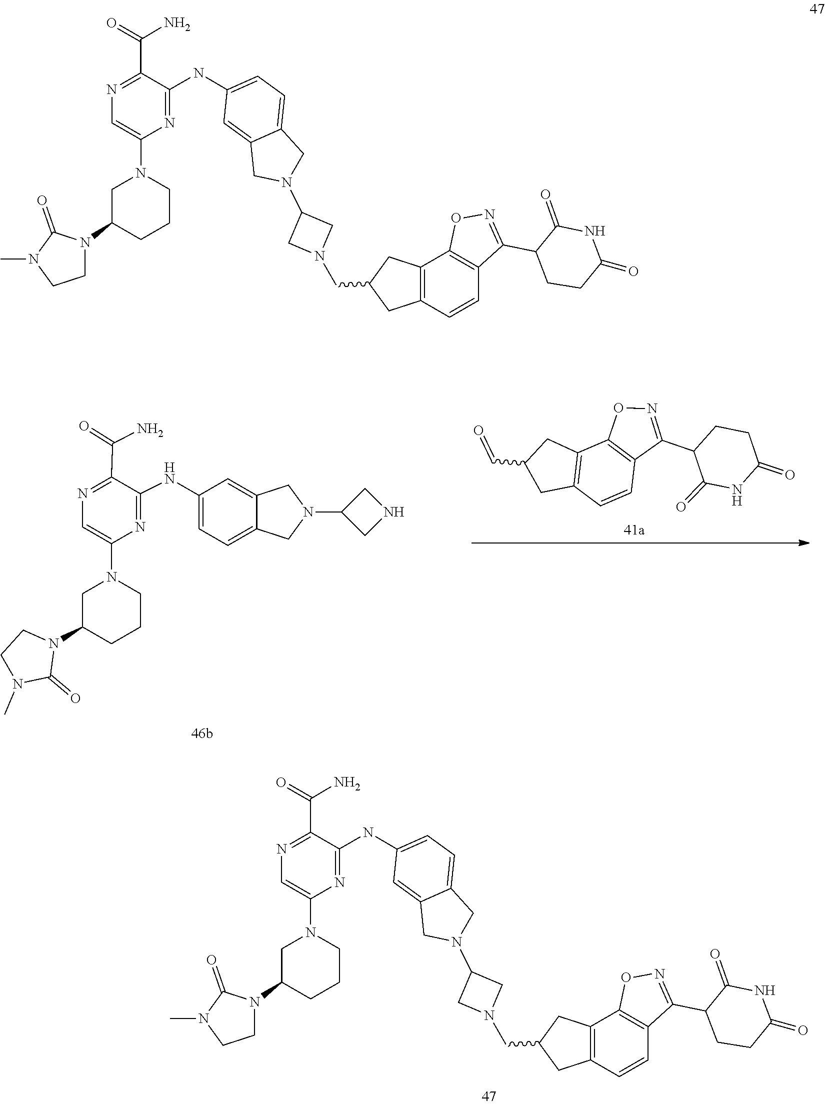

Intermediate 41a (50 mg), 1,2-dichloroethane (6 mL), isopropanol (2 mL), intermediate 46b (83 mg), acetic acid (5.00 mg, 0.083 mmol), and sodium cyanoborohydride (20.93 mg, 0.333 mmol) were added to a reaction flask in sequence, and the mixture was stirred and reacted at room temperature. After the reaction was completed, as confirmed by TLC, 2 mL of a saturated sodium bicarbonate solution was added to the reaction solution, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography to give compound 47 (30 mg).

MS(ESI, [M+H]⁺) m/z: 774.49.

¹H NMR (500 MHz, DMSO-d6) δ 11.31 (s, 1H), 11.08 (s, 1H), 7.77 (d, J=2.8 Hz, 1H), 7.66 (d, J=8.4 Hz, 2H), 7.61 (d, J=8.0 Hz, 1H), 7.36 (d, J=2.9 Hz, 1H), 7.23 (dd, J=19.6, 8.1 Hz, 2H), 7.14 (d, J=8.2 Hz, 1H), 4.56 (dd, J=11.8, 5.0 Hz, 1H), 4.37 (d, J=12.6 Hz, 1H), 4.26 (d, J=13.4 Hz, 1H), 3.78 (d, J=11.1 Hz, 4H), 3.60 (dq, J=10.1, 5.4, 4.8 Hz, 1H), 3.47 (s, 3H), 3.35 (s, 1H), 3.30-3.12 (m, 5H), 2.99 (dt, J=41.9, 11.7 Hz, 4H), 2.91-2.71 (m, 4H), 2.69 (s, 3H), 2.61 (q, J=4.2 Hz, 2H), 2.58 (t, J=4.1 Hz, 1H), 2.49-2.42 (m, 1H), 2.18 (dq, J=14.0, 5.0 Hz, 1H), 1.84-1.73 (m, 3H), 1.57 (t, J=12.1 Hz, 1H).

### Example 48: Synthesis of Compound 48

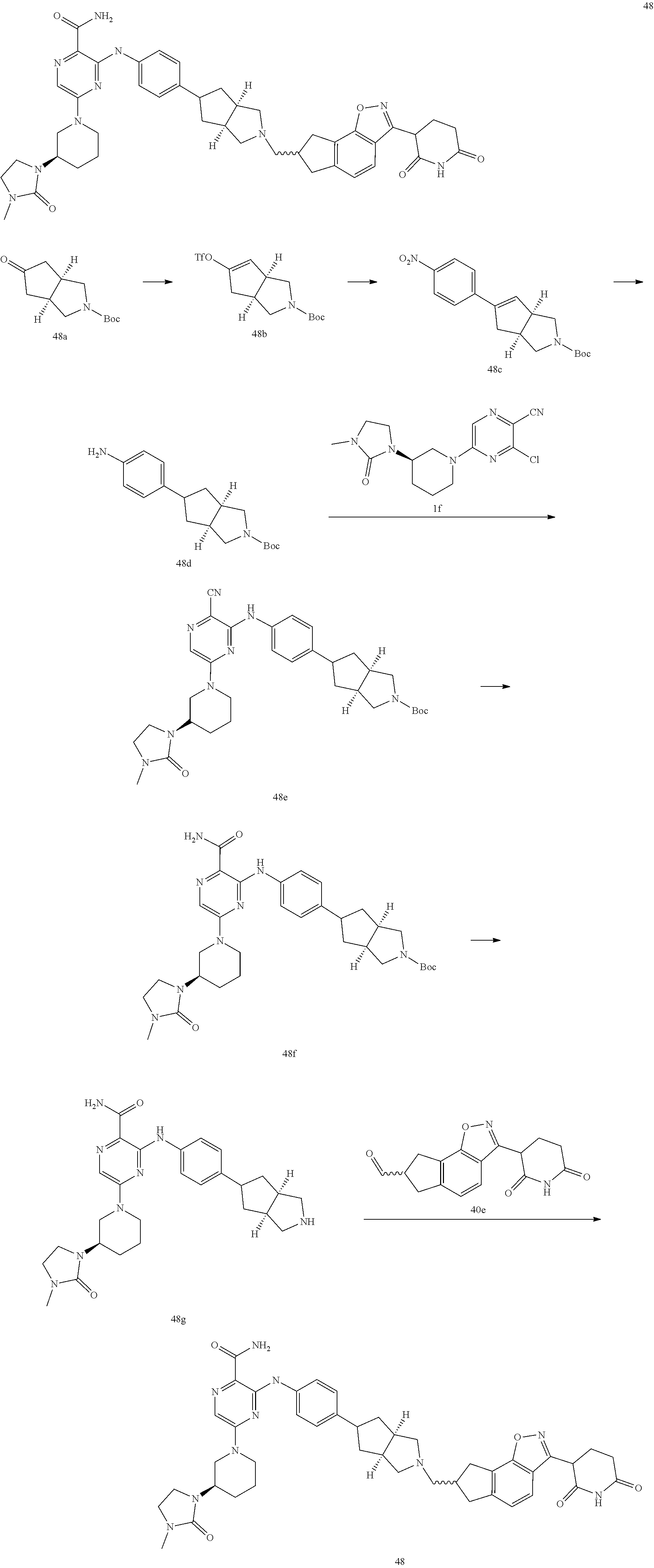

### Step 1: Preparation of Intermediate 48b

Lithium bis(trimethylsilyl)amide (26.6 mL) was added dropwise to a stirred solution of 48 (1.16 g) in THF (30 mL) at −78° C., and the mixture was maintained at this temperature and reacted for 1 h. N-phenylbis(trifluoromethanesulfonyl)imide (1.50 g) was dissolved in THF (30 mL), and the solution was added dropwise to the reaction solution. After the dropwise addition was completed, the mixture was allowed to react at room temperature. After the reaction was completed, as confirmed by TLC, the reaction solution was poured into 100 mL of water and extracted twice with petroleum ether (100 mL each time). The organic phases were combined, washed with saturated sodium chloride, dried over anhydrous sodium sulfate, filtered, and concentrated to give intermediate 48b (6.2 g).

¹H NMR (500 MHz, DMSO-d₆) δ 5.76 (d, J=2.0 Hz, 1H), 3.51 (dd, J=11.3, 8.5 Hz, 1H), 3.29 (q, J=7.4 Hz, 3H), 3.00 (dd, J=11.3, 6.1 Hz, 1H), 2.93-2.77 (m, 2H), 2.35-2.28 (m, 1H), 1.33 (s, 9H).

### Step 2: Preparation of Intermediate 48c

Intermediate 48b (5.0 g), 4-nitrophenylboronic acid (2.33 g), potassium carbonate (3.87 g), PdCl₂(dppf) (0.51 g), water (10 mL), and 1,4-dioxane (50 mL) were added to a reaction flask in sequence, and the mixture was purged three times with nitrogen, heated to 100TC and reacted. After the reaction was completed, as confirmed by TLC, the reaction solution was cooled to room temperature, and 80 L of ethyl acetate and 140 mL of water were added to the residue. The organic phase was separated, washed with a saturated sodium chloride solution, dried over anhydrous sodium sulfate, filtered, and concentrated. The concentrate was separated and purified by silica gel column chromatography to give intermediate 48c (4.2 g).

MS(ESI, [M+H]⁺) m/z: 330.21.

### Step 3: Preparation of Intermediate 48d

Intermediate 48c (0.30 g), 10% palladium on carbon (0.84 g), methanol (60 mL), and dichloromethane (30 mL) were added to a reaction flask in sequence, and the mixture was purged three times with hydrogen and reacted at room temperature. After the reaction was completed, as confirmed by TLC, palladium on carbon was removed by filtration under vacuum, and the residue was concentrated to give intermediate 48d (3.72 g).

MS(ESI, [M+H]⁺) m/z: 302.21.

### Step 4: Preparation of Intermediate 48e

Intermediate 48d (1.88 g), 1f (2.0 g), BINAP (0.39 g), cesium carbonate (6.09 g), palladium acetate (0.14 g), and dioxane (50 mL) were added to a reaction flask in sequence, and the mixture was purged three times with nitrogen, heated to 100° C., and reacted. After the reaction was completed, as confirmed by TLC, the reaction solution was cooled to room temperature, and 80 L of ethyl acetate and 140 mL of water were added to the residue. The organic phase was separated, washed with a saturated sodium chloride solution, dried over anhydrous sodium sulfate, filtered, and concentrated. The concentrate was separated and purified by silica gel column chromatography to give intermediate 4e (3.0 g).

MS(ESI, [M+H]⁺) m/z: 587.3.

### Step 5: Preparation of Intermediate 48f

Intermediate 4e (3.0 g), cesium carbonate (1.43 g), water (10 mL), DMSO (35 mL), and methanol (20 mL) were added to a reaction flask in sequence. The reaction solution was cooled to 0° C., and 30 wt % hydrogen peroxide (1.49 g) was added dropwise. After the dropwise addition was completed, the mixture was allowed to react at room temperature. After the reaction was completed, as confirmed by TLC, 100 mL of water was added to the reaction solution, and then a saturated aqueous sodium sulfite solution was added to neutralize hydrogen peroxide. The mixture was stirred for 10 min and filtered, and the filter cake was collected and dried to give intermediate 48f (2.0 g).

MS(ESI, [M+H]⁺) m/z: 605.4.

### Step 6: Preparation of Intermediate 48g

Intermediate 48f (0.45 g), dichloromethane (20 mL), and trifluoroacetic acid (3 mL) were added to a reaction flask in sequence, and the mixture was reacted at room temperature. After the reaction was completed, as confirmed by TLC, water (50 mL) was added to the reaction solution, and the pH was adjusted to about 8 with a saturated aqueous sodium bicarbonate solution. The mixture was stirred for 10 min and filtered, and the filter cake was collected and dried to give intermediate 48g (0.20 g).

MS(ESI, [M+H]⁺) m/z: 505.5.

### Step 7: Preparation of Compound 48

Intermediate 48g (85 mg), 40e (50 mg), dichloroethane/isopropanol (5:1, 20 mL), glacial acetic acid (10 mg), and sodium cyanoborohydride (21 mg) were added to a reaction flask in sequence, and the mixture was reacted at room temperature. After the reaction was completed, as confirmed by TLC, 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 48 (32 mg).

MS(ESI, [M+H]⁺) m/z: 787.5.

1H NMR (500 MHz, DMSO-d6) δ 11.22 (s, 1H), 11.08 (s, 1H), 7.76 (d, J=2.9 Hz, 1H), 7.66 (s, 1H), 7.60 (d, J=8.0 Hz, 1H), 7.52 (d, J=8.3 Hz, 2H), 7.34 (d, J=2.8 Hz, 1H), 7.26 (d, J=8.0 Hz, 1H), 7.18 (d, J=8.1 Hz, 2H), 4.56 (dd, J=11.8, 5.0 Hz, 1H), 4.40 (d, J=12.4 Hz, 1H), 4.34-4.26 (m, 1H), 3.68-3.58 (m, 1H), 3.28-3.14 (m, 4H), 3.03 (t, J=12.0 Hz, 1H), 3.00-2.90 (m, 2H), 2.90-2.74 (m, 4H), 2.72 (d, J=3.0 Hz, 3H), 2.66 (d, J=8.9 Hz, 2H), 2.59 (dd, J=17.7, 4.8 Hz, 3H), 2.48-2.42 (m, 2H), 2.30-2.13 (m, 5H), 1.89-1.72 (m, 3H), 1.65-1.53 (m, 1H), 1.41 (d, J=8.1 Hz, 2H).

### Example 49: Synthesis of Compound 49

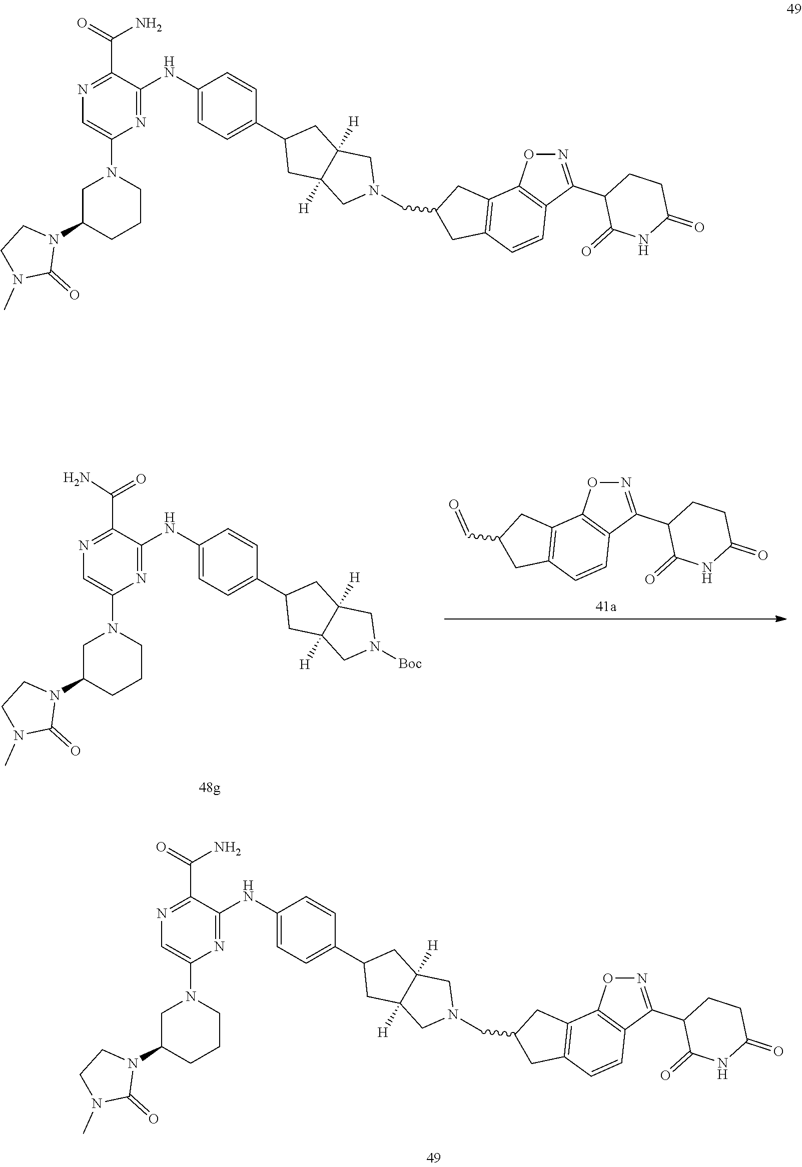 [SMILES: CN1CCN([C@@H]2CCCN(c3cnc(C(N)=O)c(Nc4ccc(C5C[C@@H]6CN(CC7Cc8ccc9c(C%10CCC(=O)NC%10=O)noc9c8C7)C[C@@H]6C5)cc4)n3)C2)C1=O ; CN1CCN([C@@H]2CCCN(c3cnc(C(N)=O)c(Nc4ccc(C5C[C@@H]6CN(C(=O)OC(C)(C)C)C[C@@H]6C5)cc4)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc(C5C[C@@H]6CN(CC7Cc8ccc9c(C%10CCC(=O)NC%10=O)noc9c8C7)C[C@@H]6C5)cc4)n3)C2)C1=O]

### Step 1: Preparation of Compound 49

Intermediate 48g (85 mg), 41a (50 mg), dichloroethane/isopropanol (5:1, 20 mL), glacial acetic acid (10 mg), and sodium cyanoborohydride (21 mg) were added to a reaction flask in sequence, and the mixture was reacted at room temperature. After the reaction was completed, as confirmed by TLC, 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 49 (50 mg).

MS(ESI, [M+H]⁺) m/z: 787.5.

1H NMR (500 MHz, DMSO-d6) δ 11.22 (s, 1H), 11.08 (s, 1H), 7.76 (d, J=2.8 Hz, 1H), 7.66 (s, 1H), 7.60 (d, J=8.0 Hz, 1H), 7.56-7.46 (m, 2H), 7.34 (d, J=2.8 Hz, 1H), 7.27 (d, J=8.0 Hz, 1H), 7.18 (d, J=8.6 Hz, 2H), 4.56 (dd, J=11.8, 4.9 Hz, 1H), 4.39 (d, J=12.6 Hz, 1H), 4.34-4.26 (m, 1H), 3.63 (t, J=11.5 Hz, 1H), 3.29-3.13 (m, 4H), 3.04 (t, J=11.7 Hz, 1H), 2.99-2.90 (m, 2H), 2.89-2.74 (m, 4H), 2.72 (d, J=3.0 Hz, 3H), 2.66 (d, J=8.8 Hz, 2H), 2.63-2.54 (m, 3H), 2.46 (d, J=12.4 Hz, 2H), 2.27-2.14 (m, 5H), 1.90-1.72 (m, 3H), 1.60-1.54 (m, 1H), 1.47-1.32 (m, 3H).

### Example 50: Synthesis of Compound 50

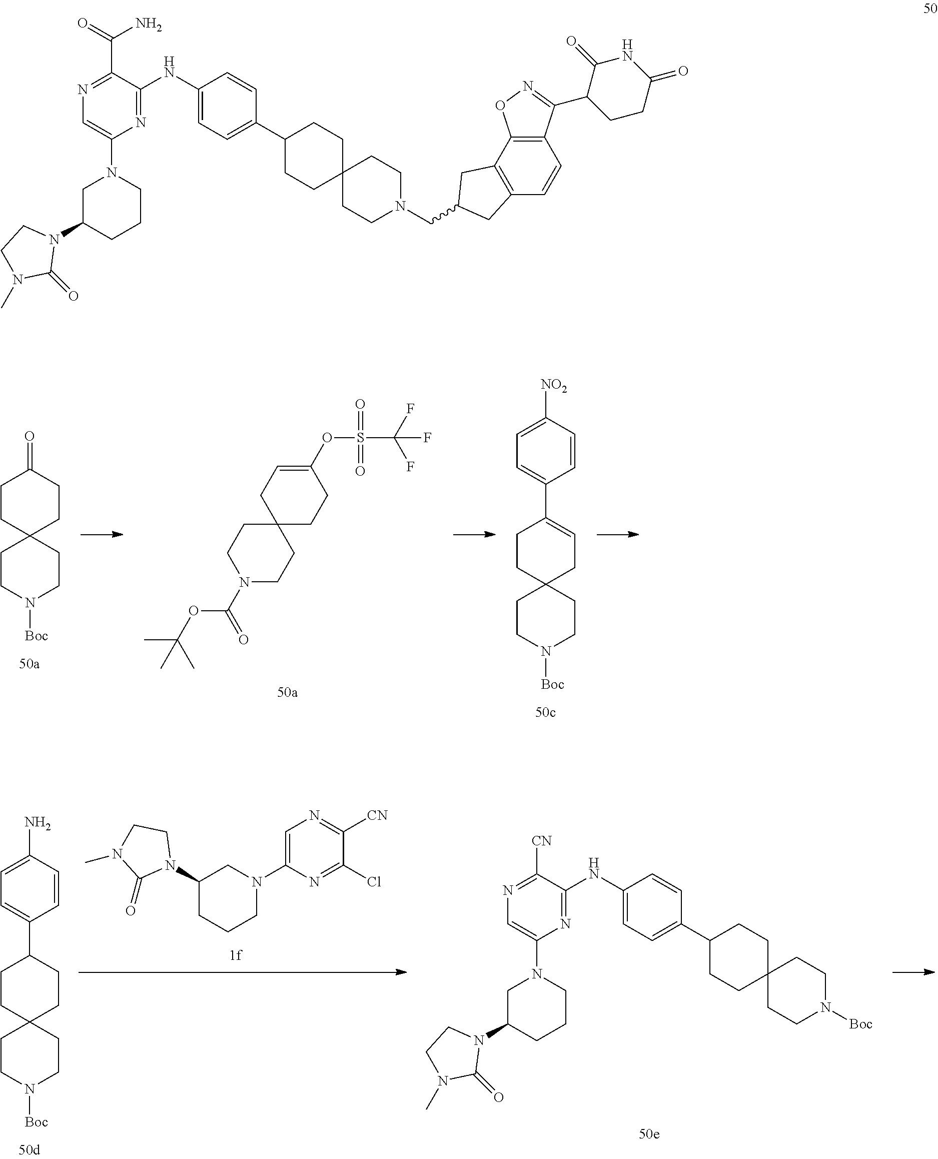

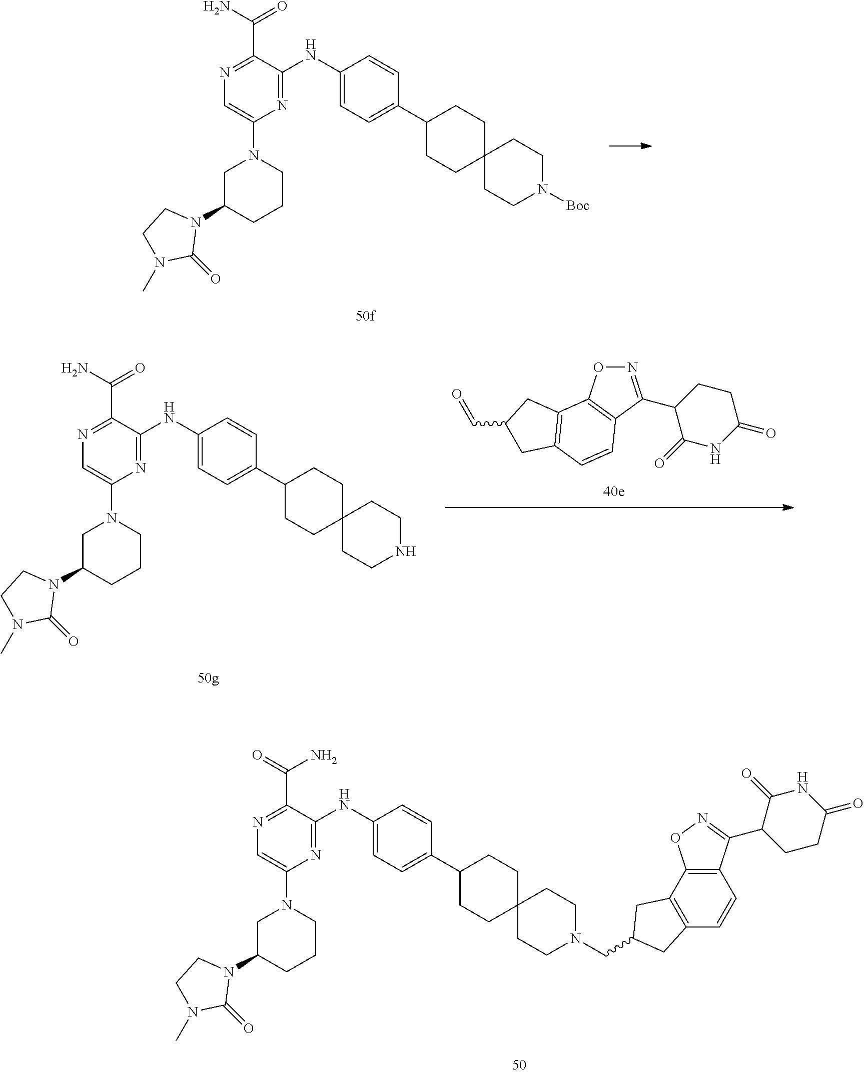 [SMILES: CN1CCN([C@@H]2CCCN(c3cnc(C(N)=O)c(Nc4ccc(C5CCC6(CC5)CCN(C(=O)OC(C)(C)C)CC6)cc4)n3)C2)C1=O ; CN1CCN([C@@H]2CCCN(c3cnc(C(N)=O)c(Nc4ccc(C5CCC6(CCNCC6)CC5)cc4)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc(C5CCC6(CC5)CCN(CC5Cc7ccc8c(C9CCC(=O)NC9=O)noc8c7C5)CC6)cc4)n3)C2)C1=O]

### Step 1: Preparation of Intermediate 50b

Lithium bis(trimethylsilyl)amide (2.82 g, 16.85 mL) was slowly added dropwise to a solution of 50a (3 g) in THF (30 mL) at −78° C. under nitrogen atmosphere. After the dropwise addition was completed, the mixture was reacted at −78° C. for 1 h. A solution of N-phenylbis(trifluoromethanesulfonyl)imide (4.81 g) in THF (30 mL) was slowly added to the reaction solution. After the addition was completed, the mixture was warmed to room temperature and reacted. After the reaction was completed, 200 mL of petroleum ether was added to the system, and 200 mL of saturated brine was added for extraction. The organic phase was dried over anhydrous sodium sulfate, filtered, and concentrated to give 50b (4.51 g).

¹H NMR (500 MHz, DMSO-d6) δ 5.64 (td, J=4.1, 2.0 Hz, 1H), 3.18 (dd, J=13.6, 5.9 Hz, 2H), 3.11 (dd, J=13.3, 6.4 Hz, 2H), 2.13 (tq, J=6.4, 2.0 Hz, 2H), 1.91 (dt, J=4.9, 2.7 Hz, 2H), 1.46 (t, J=6.5 Hz, 2H), 1.22 (s, 9H), 1.14 (t, J=5.9 Hz, 4H).

### Step 2: Preparation of Intermediate 50c

4-nitrophenylboronic acid (1.6 g), 50b (4.59 g), potassium carbonate (3.97 g), Pd(dppf)₂Cl₂ (0.783 g), 1,4-dioxane (30 mL), and water (8 mL) were added to a reaction flask in sequence, and the mixture was purged several times with nitrogen, heated to 90° C. and reacted. After the reaction was completed, about 200 mL of methyl tert-butyl ether was added, and the mixture was washed with saturated brine. The organic phase was dried over anhydrous sodium sulfate, filtered, and concentrated to give a crude product, and the crude product was slurried with petroleum ether to give 50c (3.83 g).

¹H NMR (500 MHz, DMSO-d6) δ 8.08-7.94 (m, 2H), 7.59-7.46 (m, 2H), 6.24 (td, J=4.1, 2.0 Hz, 1H), 3.23 (dt, J=11.9, 5.6 Hz, 2H), 3.11 (d, J=7.4 Hz, 2H), 2.29-2.18 (m, 2H), 1.98 (dt, J=4.6, 2.5 Hz, 2H), 1.46 (t, J=6.4 Hz, 2H), 1.23 (s, 9H), 1.17 (t, J=5.8 Hz, 4H).

### Step 3: Preparation of Intermediate 50d

50c (3.83 g), 10% palladium on carbon (1 g), and methanol (50 mL) were added to a reaction flask in sequence, and the mixture was purged 3 times with hydrogen and reacted overnight at room temperature under hydrogen atmosphere. After the reaction was completed, the reaction solution was filtered under vacuum, and the filtrate was concentrated to give 50d (3.6 g).

¹H NMR (500 MHz, DMSO-d6) δ 6.87 (d, J=8.1 Hz, 2H), 6.47 (d, J=8.0 Hz, 2H), 4.85 (s, 2H), 3.30 (q, J=5.9 Hz, 4H), 2.26 (tt, J=10.2, 4.8 Hz, 1H), 1.73 (d, J=13.0 Hz, 2H), 1.50 (pd, J=7.7, 7.1, 2.8 Hz, 6H), 1.39 (s, 9H), 1.24 (dd, J=7.0, 4.5 Hz, 2H), 1.16 (td, J=13.0, 5.1 Hz, 2H).

### Step 4: Preparation of Intermediate 50e

Intermediate 1f (1.6 g), 50d (2.84 g), cesium carbonate (4.88 g), BINAP (0.311 g), palladium acetate (0.112 g), and 1,4-dioxane (10 mL) were added to a single-necked flask in sequence, and the mixture was heated to 100° C. and reacted under nitrogen atmosphere. After the reaction was completed, the reaction solution was cooled to room temperature and purified by silica gel column chromatography to give 50e (2.6 g).

¹H NMR (500 MHz, DMSO-d6) δ 8.93 (s, 1H), 7.81 (s, 1H), 7.48-7.41 (m, 2H), 7.13 (d, J=8.5 Hz, 2H), 4.41-4.16 (m, 2H), 3.56 (q, J=6.0, 4.9 Hz, 1H), 3.23 (ddd, J=10.9, 7.6, 4.7 Hz, 8H), 2.91 (dd, J=21.2, 9.0 Hz, 2H), 2.70 (s, 3H), 2.45-2.35 (m, 1H), 1.83-1.70 (m, 5H), 1.64-1.46 (m, 7H), 1.39 (s, 9H), 1.30-1.17 (m, 4H).

### Step 5: Preparation of Intermediate 50f

Intermediate 50e (2.5 g), DMSO (10 mL), and cesium carbonate (1.29 g) were added to a single-necked flask in sequence. 30 wt % hydrogen peroxide (2.254 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 50f (2.41 g).

MS(ESI, [M+H]⁺) m/z: 647.47.

### Step 6: Preparation of Intermediate 50g

50f (2.4 g), dichloromethane (10 mL), and TFA (8.46 g, 5.72 mL, 74.2 mmol) were added to a single-necked flask in sequence, and the mixture was reacted at room temperature. After the reaction was completed, the system was added to 200 mL of a saturated sodium bicarbonate solution, and then 200 mL of dichloromethane was added for extraction. The organic phase was dried over anhydrous sodium sulfate, filtered, and concentrated by evaporation at reduced pressure to remove the solvent, thus giving 50g (1.73 g).

MS(ESI, [M+H]+) m/z: 547.48.

### Step 7: Preparation of Compound 50

40e (60 mg), isopropanol (1 mL), 1,2-dichloroethane (5.00 mL), and 50g (109 mg) were added to a reaction flask in sequence, and sodium cyanoborohydride (25.1 mg) was added. The mixture was reacted at room temperature. After the reaction was completed, the reaction solution was purified by silica gel column chromatography to give 50 (56 mg).

MS(ESI, [M+H]⁺) m/z: 829.57

¹H NMR (500 MHz, DMSO-d6) δ 11.20 (s, 1H), 11.09 (s, 1H), 7.75 (d, J=2.9 Hz, 1H), 7.65 (s, 1H), 7.61 (d, J=8.1 Hz, 1H), 7.49 (d, J=8.1 Hz, 2H), 7.33 (d, J=2.9 Hz, 1H), 7.27 (d, J=8.1 Hz, 1H), 7.16 (d, J=8.2 Hz, 2H), 4.63-4.51 (m, 1H), 4.44-4.23 (m, 2H), 3.61 (dt, J=11.1, 6.6 Hz, 1H), 3.30 (d, J=8.0 Hz, 1H), 3.27-3.13 (m, 5H), 3.11-2.73 (m, 7H), 2.71 (s, 3H), 2.66-2.52 (m, 4H), 2.49-2.33 (m, 4H), 2.18 (dt, J=13.2, 4.6 Hz, 1H), 1.90-1.71 (m, 5H), 1.70-1.47 (m, 7H), 1.41 (s, 2H), 1.26-1.15 (m, 2H).

### Example 51: Synthesis of Compound 51

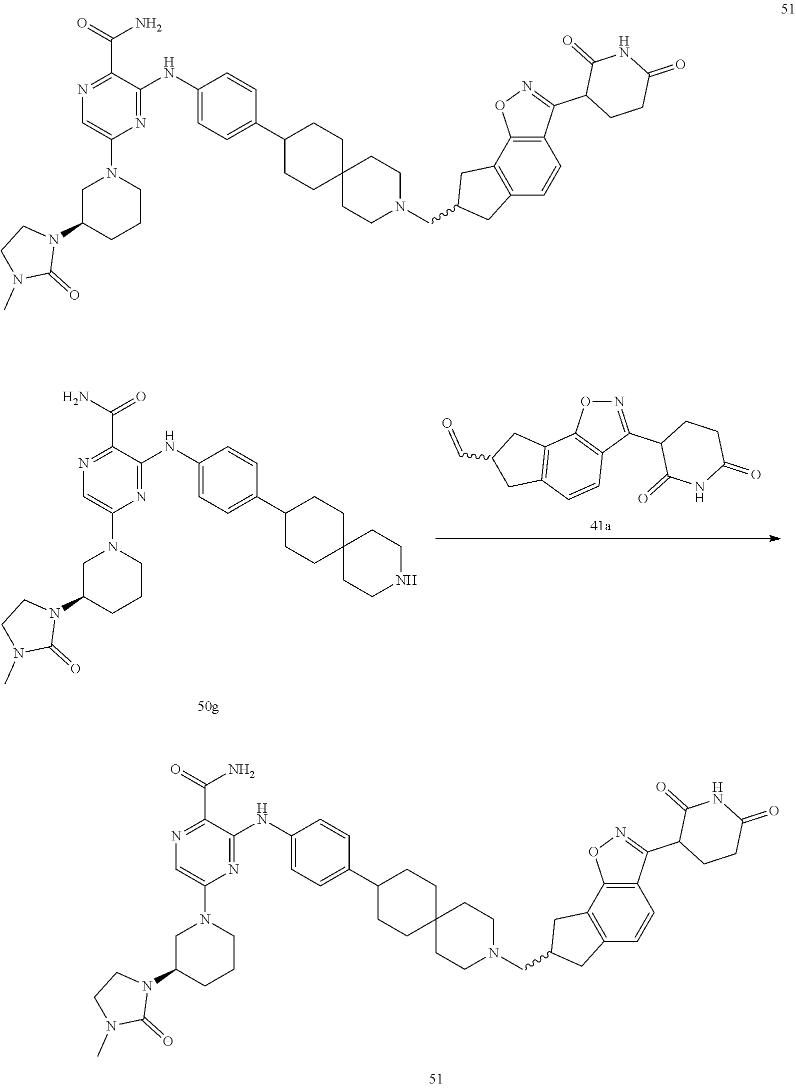 [SMILES: CN1CCN([C@@H]2CCCN(c3cnc(C(N)=O)c(Nc4ccc(C5CCC6(CC5)CCN(CC5Cc7ccc8c(C9CCC(=O)NC9=O)noc8c7C5)CC6)cc4)n3)C2)C1=O ; CN1CCN([C@@H]2CCCN(c3cnc(C(N)=O)c(Nc4ccc(C5CCC6(CCNCC6)CC5)cc4)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc(C5CCC6(CC5)CCN(CC5Cc7ccc8c(C9CCC(=O)NC9=O)noc8c7C5)CC6)cc4)n3)C2)C1=O]

### Step 1: Preparation of Compound 51

41a (60 mg), isopropanol (1 mL), 1,2-dichloroethane (5.00 mL), and 50g (109 mg) were added to a reaction flask in sequence, and sodium cyanoborohydride (25.1 mg) was added. The mixture was reacted at room temperature. After the reaction was completed, the reaction solution was purified by silica gel column chromatography to give 51 (42 mg).

MS(ESI, [M+H]+) m/z: 829.59.

¹H NMR (500 MHz, DMSO-4) δ 11.20 (s, 1H), 11.09 (s, 1H), 7.75 (d, J=2.9 Hz, 1H), 7.65 (s, 1H), 7.61 (d, J=8.1 Hz, 1H), 7.49 (d, J=8.1 Hz, 2H), 7.33 (d, J=2.9 Hz, 1H), 7.27 (d, J=8.1 Hz, 1H), 7.16 (d, J=8.2 Hz, 2H), 4.63-4.51 (m, 1H), 4.44-4.23 (m, 2H), 3.61 (dt, J=11.1, 6.6 Hz, 1H), 3.30 (d, J=8.0 Hz, 1H), 3.27-3.13 (m, 5H), 3.11-2.73 (m, 7H), 2.71 (s, 3H), 2.66-2.52 (m, 4H), 2.49-2.33 (m, 4H), 2.18 (dt, J=13.2, 4.6 Hz, 1H), 1.90-1.71 (m, 5H), 1.70-1.47 (m, 7H), 1.41 (s, 2H), 1.26-1.15 (m, 2H).

### Example 52: Synthesis of Compound 52

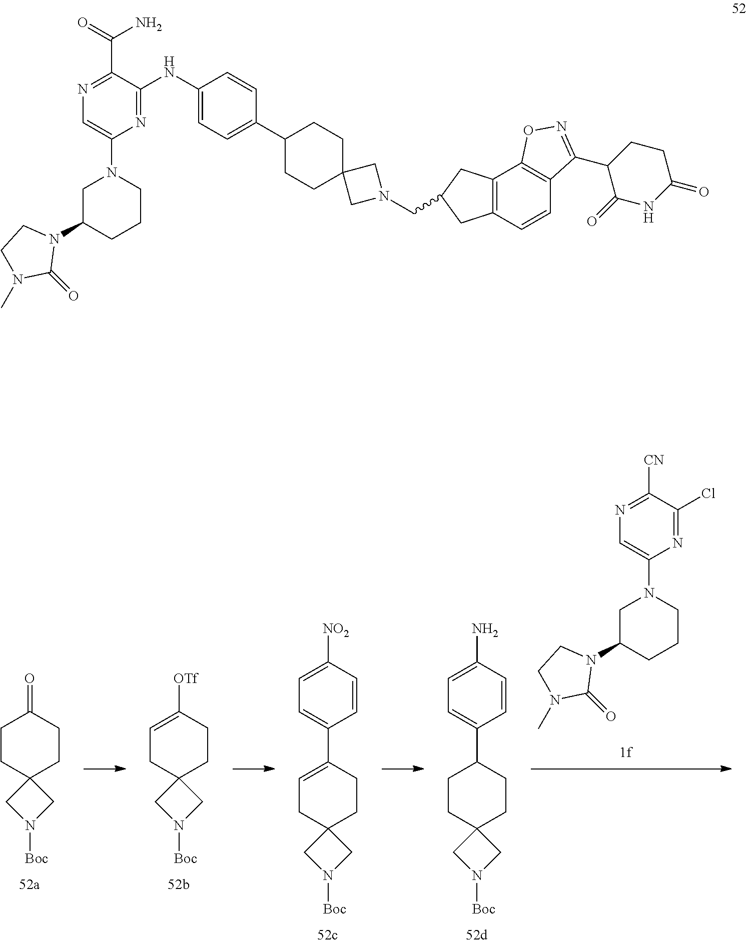

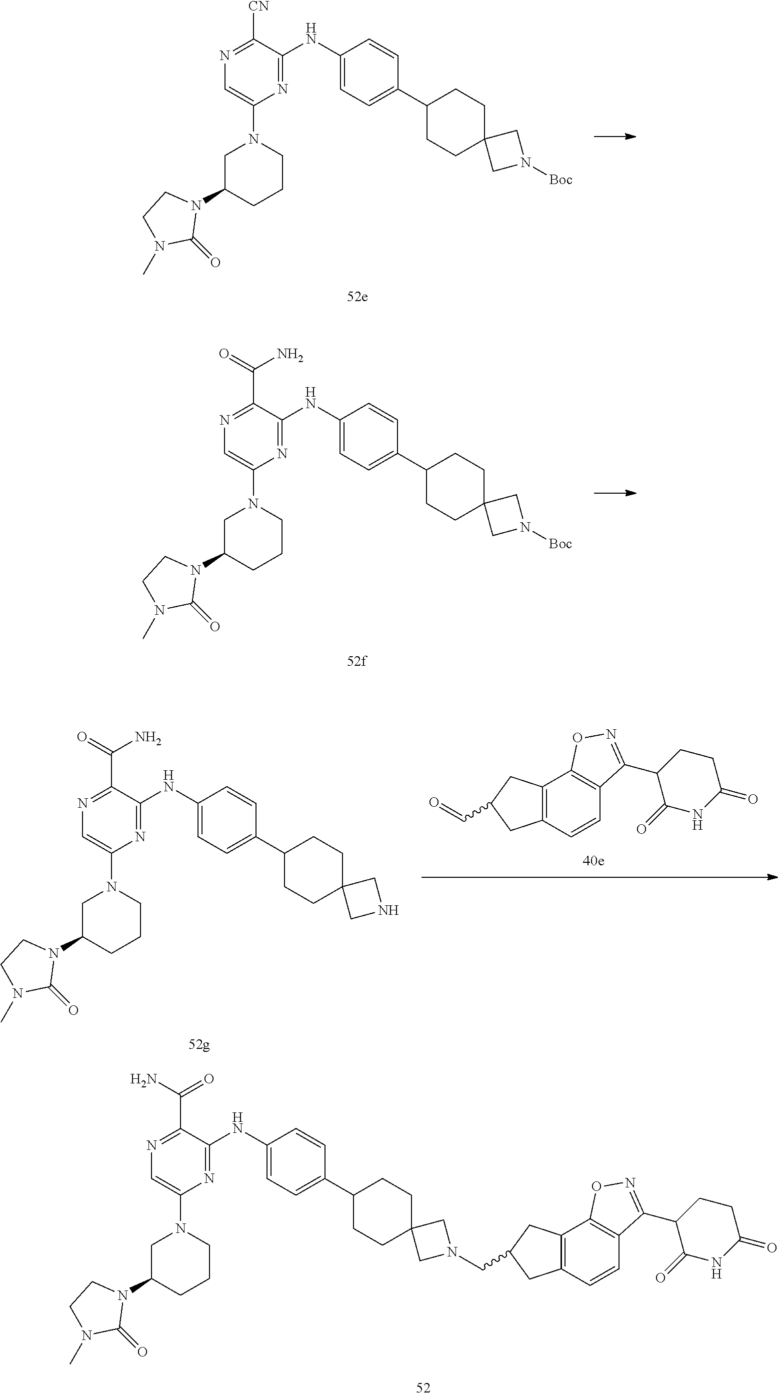 [SMILES: CN1CCN([C@@H]2CCCN(c3cnc(C#N)c(Nc4ccc(C5CCC6(CC5)CN(C(=O)OC(C)(C)C)C6)cc4)n3)C2)C1=O ; CN1CCN([C@@H]2CCCN(c3cnc(C(N)=O)c(Nc4ccc(C5CCC6(CC5)CN(C(=O)OC(C)(C)C)C6)cc4)n3)C2)C1=O ; CN1CCN([C@@H]2CCCN(c3cnc(C(N)=O)c(Nc4ccc(C5CCC6(CC5)CNC6)cc4)n3)C2)C1=O ; O=CC1Cc2ccc3c(C4CCC(=O)NC4=O)noc3c2C1 ; CN1CCN([C@@H]2CCCN(c3cnc(C(N)=O)c(Nc4ccc(C5CCC6(CC5)CN(CC5Cc7ccc8c(C9CCC(=O)NC9=O)noc8c7C5)C6)cc4)n3)C2)C1=O]

### Step 1: Preparation of Intermediate 52b

Lithium bis(trimethylsilyl)amide (1 M, 21.75 mL) was slowly added dropwise to a solution of 52a (4 g) in THF (30 mL) at −78° C. under nitrogen atmosphere. After the dropwise addition was completed, the mixture was reacted at −78° C. for 1 h. A solution of N-phenylbis(trifluoromethanesulfonyl)imide (7.17 g) in THF (30 mL) was slowly added to the reaction solution. After the addition was completed, the mixture was warmed to room temperature and reacted. After the reaction was completed, 200 mL of petroleum ether was added to the system, and 200 mL of saturated brine was added for extraction. The organic phase was dried over anhydrous sodium sulfate, filtered, and concentrated to give 52b (5 g).

¹H NMR (500 MHz, DMSO-d6) δ 5.88-5.77 (m, 1H), 3.62 (s, 2H), 3.52 (d, J=8.1 Hz, 2H), 2.40 (d, J=4.2 Hz, 4H), 1.90 (t, J=6.1 Hz, 2H), 1.37 (s, 9H).

### Step 2: Preparation of Intermediate 52c

4-nitrophenylboronic acid (5.03 g), 52b (5 g), potassium carbonate (5.58 g), Pd(dppf)₂Cl₂ (2.199 g), 1,4-dioxane (50 mL), and water (10 mL) were added to a reaction flask in sequence, and the mixture was purged several times with nitrogen, heated to 90° C., and reacted. After the reaction was completed, about 200 mL of methyl tert-butyl ether was added, and the mixture was extracted with 200 mL of saturated brine. The organic phase was dried over anhydrous sodium sulfate, filtered, and concentrated to give a crude product, and the crude product was slurred with petroleum ether to give 50c (4 g).

¹H NMR (500 MHz, DMSO-d6) δ58.23-8.12 (m, 2H), 7.75-7.62 (m, 2H), 6.37 (td, J=3.9, 1.9 Hz, 1H), 3.65 (s, 2H), 3.56 (d, J=6.8 Hz, 2H), 2.52 (t, J=4.2 Hz, 2H), 2.46 (dt, J=4.6, 2.4 Hz, 2H), 1.89 (t, J=6.3 Hz, 2H), 1.38 (s, 9H).

### Step 3: Preparation of Intermediate 52d

52c (2 g), 10% palladium on carbon (300 mg), and methanol (30 mL) were added to a reaction flask in sequence, and the mixture was purged 3 times with hydrogen and reacted overnight at room temperature under hydrogen atmosphere. After the reaction was completed, palladium on carbon was removed by filtration under vacuum, and the filtrate was concentrated to give 52d (1.92 g).

¹H NMR (500 MHz, DMSO-d6) δ 6.89-6.80 (m, 2H), 6.52-6.41 (m, 2H), 4.79 (s, 2H), 3.59 (s, 2H), 3.52-3.40 (m, 2H), 2.24 (ddd, J=12.0, 8.6, 3.4 Hz, 1H), 1.87 (d, J=12.7 Hz, 2H), 1.67-1.56 (m, 2H), 1.48 (td, J=12.9, 3.5 Hz, 2H), 1.38 (s, 9H), 1.31 (qd, J=12.9, 3.0 Hz, 2H).

### Step 4: Preparation of Intermediate 52e

Intermediate 1f (2 g), 52d (1.915 g), cesium carbonate (5.91 g), BINAP (0.377 g), palladium acetate (0.136 g), and 1,4-dioxane (20 mL) were added to a single-necked flask in sequence, and the mixture was heated to 100° C. and reacted under nitrogen atmosphere. After the reaction was completed, the reaction solution was cooled to room temperature and purified by silica gel column chromatography to give 52e (2.58 g).

¹H NMR (500 MHz, DMSO-d6) δ 8.93 (s, 1H), 7.81 (s, 1H), 7.44 (d, J=8.3 Hz, 2H), 7.10 (d, J=8.2 Hz, 2H), 5.76 (s, 2H), 4.23 (d, J=13.2 Hz, 2H), 3.70-3.42 (m, 5H), 3.28-3.19 (m, 4H), 2.94 (d, J=14.8 Hz, 2H), 2.71 (s, 3H), 2.43-2.29 (m, 1H), 1.91 (d, J=12.7 Hz, 2H), 1.82-1.66 (m, 5H), 1.59-1.47 (m, 3H), 1.38 (s, 9H).

### Step 5: Preparation of Intermediate 52f

Intermediate 52e (2.5 g), DMSO (10 mL), and cesium carbonate (1.36 g) were added to a single-necked flask in sequence. 30 wt % hydrogen peroxide (2.3 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 52f (1.86 g).

MS(ESI, [M+H]+) m/z: 619.44.

### Step 6: Preparation of Intermediate 52g

52f (1.86 g), dichloromethane (20 mL), and TFA (6.95 mL) were added to a single-necked flask in sequence, and the mixture was reacted at room temperature. After the reaction was completed, the system was added to 200 mL of a saturated sodium bicarbonate solution, and then 200 mL of dichloromethane was added for extraction. The organic phase was dried over anhydrous sodium sulfate, filtered, and concentrated by evaporation at reduced pressure to remove the solvent, thus giving 52g (1.6 g).

MS(ESI, [M+H]+) m/z: 519.35.

### Step 7: Preparation of Compound 52

40e (60 mg), isopropanol (1 mL), 1,2-dichloroethane (5.00 mL), and 52g (104 mg) were added to a reaction flask in sequence, and sodium cyanoborohydride (25.1 mg) was added. The mixture was reacted at room temperature. After the reaction was completed, the reaction solution was purified by silica gel column chromatography to give 52 (35 mg).

MS(ESI, [M+H]+) m/z: 801.66.

¹H NMR (500 MHz, DMSO-d₆) δ 11.20 (s, 1H), 11.09 (s, 1H), 7.75 (d, J=2.8 Hz, 1H), 7.65 (s, 1H), 7.61 (d, J=8.1 Hz, 1H), 7.49 (d, J=8.2 Hz, 2H), 7.33 (d, J=2.9 Hz, 1H), 7.26 (d, J=8.1 Hz, 1H), 7.13 (d, J=8.2 Hz, 2H), 4.57 (dd, J=11.8, 5.0 Hz, 1H), 4.33 (dd, J=41.6, 13.0 Hz, 2H), 3.61 (ddt, J=11.2, 8.4, 4.6 Hz, 1H), 3.45-3.33 (m, 3H), 3.30-3.09 (m, 5H), 3.09-2.91 (m, 4H), 2.91-2.82 (m, 2H), 2.72 (s, 5H), 2.60 (dt, J=17.1, 4.3 Hz, 2H), 2.55-2.51 (m, 1H), 2.49-2.44 (m, 1H), 2.44-2.35 (m, 1H), 2.22-2.14 (m, 1H), 1.99 (s, 2H), 1.86-1.79 (m, 2H), 1.79-1.73 (m, 1H), 1.71 (dd, J=12.2, 7.7 Hz, 2H), 1.61-1.47 (m, 3H), 1.39 (d, J=12.7 Hz, 2H).

### Example 53: Synthesis of Compound 53

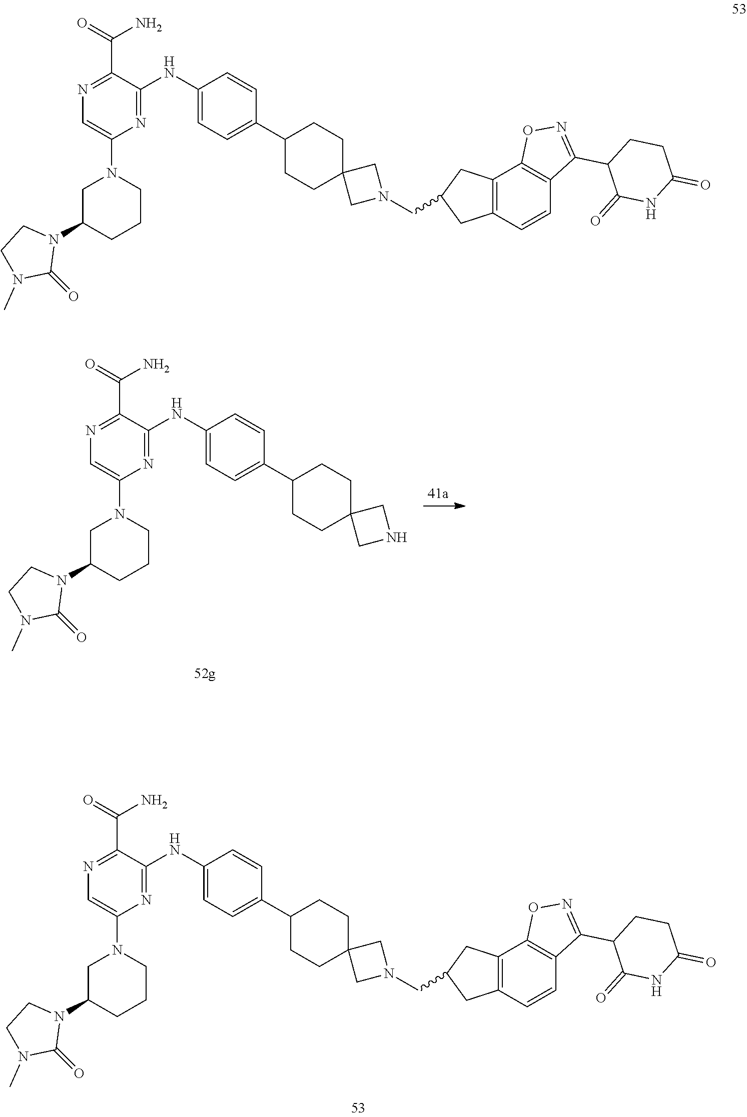 [SMILES: CN1CCN([C@@H]2CCCN(c3cnc(C(N)=O)c(Nc4ccc(C5CCC6(CC5)CN(CC5Cc7ccc8c(C9CCC(=O)NC9=O)noc8c7C5)C6)cc4)n3)C2)C1=O ; CN1CCN([C@@H]2CCCN(c3cnc(C(N)=O)c(Nc4ccc(C5CCC6(CC5)CNC6)cc4)n3)C2)C1=O ; CN1CCN([C@@H]2CCCN(c3cnc(C(N)=O)c(Nc4ccc(C5CCC6(CC5)CN(CC5Cc7ccc8c(C9CCC(=O)NC9=O)noc8c7C5)C6)cc4)n3)C2)C1=O]

### Step 1: Preparation of Compound 53

41a (60 mg), isopropanol (1 mL), 1,2-dichloroethane (5.00 mL), and 52g (104 mg) were added to a reaction flask in sequence, and sodium cyanoborohydride (25.1 mg) was added. The mixture was reacted at room temperature. After the reaction was completed, the reaction solution was purified by silica gel column chromatography to give 53 (45 mg).

MS(ESI, [M+H]+) m/z: 801.53.

¹H NMR (500 MHz, DMSO-d₆) δ 11.20 (s, 1H), 11.09 (s, 1H), 7.75 (d, J=2.8 Hz, 1H), 7.65 (s, 1H), 7.61 (d, J=8.1 Hz, 1H), 7.49 (d, J=8.2 Hz, 2H), 7.33 (d, J=2.9 Hz, 1H), 7.26 (d, J=8.1 Hz, 1H), 7.13 (d, J=8.2 Hz, 2H), 4.57 (dd, J=11.8, 5.0 Hz, 1H), 4.33 (dd, J=41.6, 13.0 Hz, 2H), 3.61 (ddt, J=11.2, 8.4, 4.6 Hz, 1H), 3.45-3.33 (m, 3H), 3.30-3.09 (m, 5H), 3.09-2.91 (m, 4H), 2.91-2.82 (m, 2H), 2.72 (s, 5H), 2.60 (dt, J=17.1, 4.3 Hz, 2H), 2.55-2.51 (m, 1H), 2.49-2.44 (m, 1H), 2.44-2.35 (m, 1H), 2.22-2.14 (m, 1H), 1.99 (s, 2H), 1.86-1.79 (m, 2H), 1.79-1.73 (m, 1H), 1.71 (dd, J=12.2, 7.7 Hz, 2H), 1.61-1.47 (m, 3H), 1.39 (d, J=12.7 Hz, 2H).

### Example 54: Synthesis of Compound 54

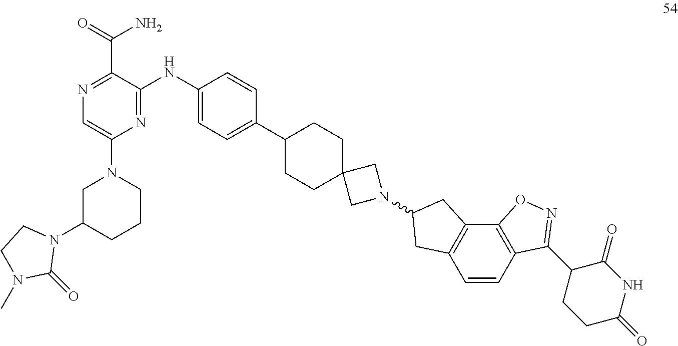 [SMILES: CN1CCN(C2CCCN(c3cnc(C(N)=O)c(Nc4ccc(C5CCC6(CC5)CN(C5Cc7ccc8c(C9CCC(=O)NC9=O)noc8c7C5)C6)cc4)n3)C2)C1=O]

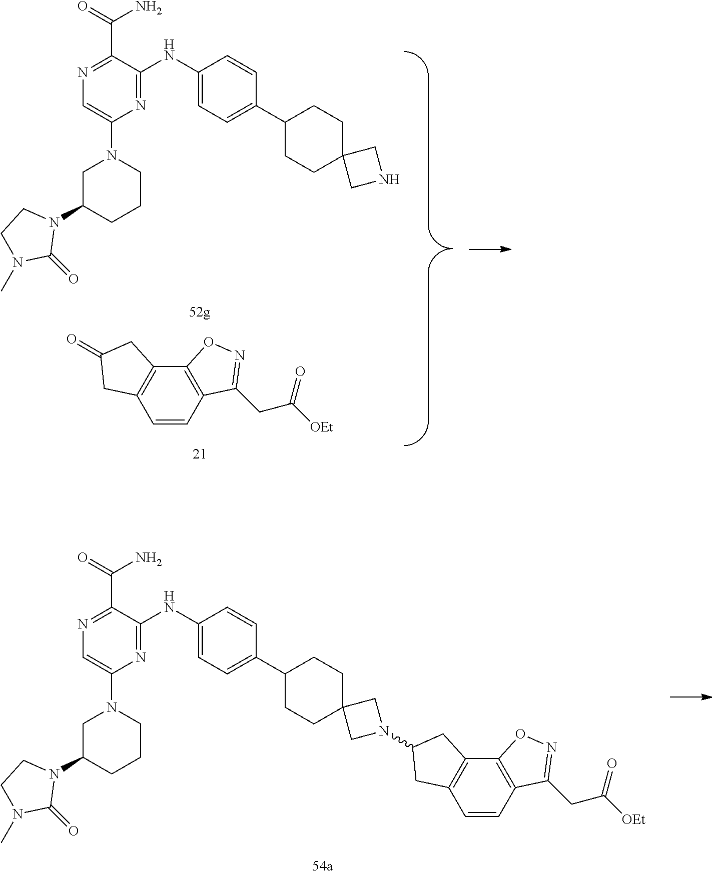 [SMILES: CN1CCN([C@@H]2CCCN(c3cnc(C(N)=O)c(Nc4ccc(C5CCC6(CC5)CNC6)cc4)n3)C2)C1=O ; CCOC(=O)Cc1noc2c3c(ccc12)CC(=O)C3 ; CCOC(=O)Cc1noc2c3c(ccc12)CC(N1CC2(CCC(c4ccc(Nc5nc(N6CCC[C@@H](N7CCN(C)C7=O)C6)cnc5C(N)=O)cc4)CC2)C1)C3]

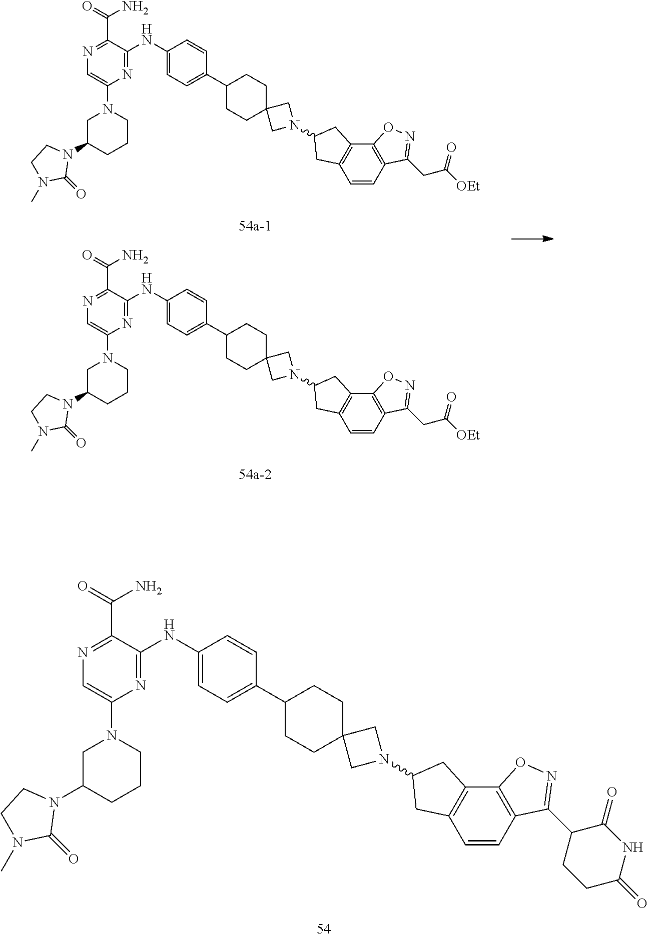 [SMILES: CCOC(=O)Cc1noc2c3c(ccc12)CC(N1CC2(CCC(c4ccc(Nc5nc(N6CCC[C@@H](N7CCN(C)C7=O)C6)cnc5C(N)=O)cc4)CC2)C1)C3 ; CCOC(=O)Cc1noc2c3c(ccc12)CC(N1CC2(CCC(c4ccc(Nc5nc(N6CCC[C@@H](N7CCN(C)C7=O)C6)cnc5C(N)=O)cc4)CC2)C1)C3 ; CN1CCN(C2CCCN(c3cnc(C(N)=O)c(Nc4ccc(C5CCC6(CC5)CN(C5Cc7ccc8c(C9CCC(=O)NC9=O)noc8c7C5)C6)cc4)n3)C2)C1=O]

### Step 1: Preparation of Intermediates 54a-1 and 54a-2

Intermediate 21 (300 mg), intermediate 52g (600 mg), 1,2-dichloroethane (10 mL), isopropanol (3 mL), and sodium cyanoborohydride (291 mg) were added to a reaction flask in sequence, and the mixture was reacted overnight at room temperature. After the reaction was completed, 5 mL of a saturated sodium bicarbonate solution was added to the reaction solution, and then dichloromethane and saturated brine were added for extraction. The organic phase was separated, and the aqueous phase was extracted with dichloromethane. The organic phases were combined, washed with saturated brine, dried over anhydrous sodium sulfate, and concentrated, and the residue was purified by silica gel column chromatography to give 54a. 54a was separated by high performance liquid chromatography to give intermediate prepeak 54a-1 (170 mg) and postpeak 54a-2 (160 mg) in sequence.

The conditions of preparative chromatography were as follows: instrument and preparative column: YMC high pressure preparative chromatograph was used, and the preparative column was CHIRALART Cellulose-SC. Mobile phase system: ethanol-dichloromethane (15:70)/n-hexane-0.1% diethylamine, isocratic elution: ethanol-dichloromethane (3:2)/n-hexane-0.1% diethylamine=85/15.

54a-1, 54a-2: MS(ESI, [M+H]⁺) m/z: 762.59.

### Step 2: Preparation of Compound 54

Intermediate 54a-1 (100 mg), acrylamide (15 mg) and tetrahydrofuran (5 mL) were added to a reaction flask in sequence, and the mixture was cooled to 0° C. under N₂ atmosphere. Potassium tert-butoxide (1 M, 0.079 mL) was added, and the mixture was reacted at 0° C. After the reaction was completed, as confirmed by TLC, the reaction solution was added dropwise to an icy saturated aqueous ammonium chloride solution, and 50 mL of ethyl acetate was added for extraction. The organic phase was dried over anhydrous sodium sulfate, filtered, and concentrated, and the residue was purified by silica gel column chromatography to give compound 54 (25 mg).

MS(ESI, [M+H]+) m/z: 787.36.

¹H NMR (500 MHz, DMSO-d6) δ 11.20 (s, 1H), 11.09 (d, J=1.5 Hz, 1H), 7.75 (d, J=2.8 Hz, 1H), 7.65 (s, 1H), 7.62 (d, J=7.4 Hz, 1H), 7.48 (d, J=8.3 Hz, 2H), 7.33 (d, J=2.8 Hz, 1H), 7.26 (d, J=8.0 Hz, 1H), 7.12 (d, J=8.2 Hz, 2H), 4.57 (dd, J=11.9, 5.0 Hz, 1H), 4.32 (dd, J=40.5, 12.7 Hz, 2H), 3.65-3.56 (m, 1H), 3.43-3.33 (m, 2H), 3.26 (dd, J=9.0, 7.3 Hz, 2H), 3.18-2.89 (m, 9H), 2.77 (ddd, J=17.1, 11.9, 5.2 Hz, 3H), 2.71 (s, 3H), 2.60 (dq, J=17.4, 4.0 Hz, 1H), 2.51-2.53 (m, 1H), 2.42-2.33 (m, 1H), 2.25-2.13 (m, 1H), 1.99-1.86 (m, 2H), 1.85-1.79 (m, 2H), 1.76 (dt, J=7.0, 3.1 Hz, 1H), 1.67 (d, J=12.1 Hz, 2H), 1.61-1.44 (m, 3H), 1.44-1.32 (m, 2H).

### Example 55: Synthesis of Compound 55

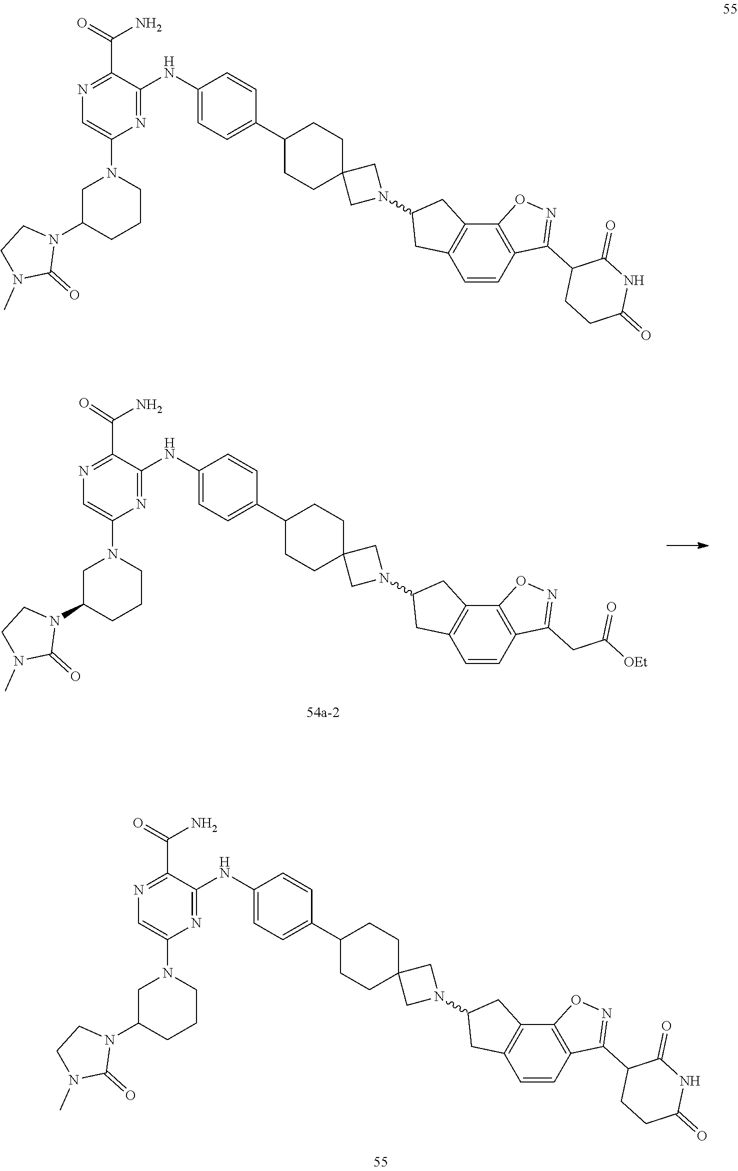 [SMILES: CN1CCN(C2CCCN(c3cnc(C(N)=O)c(Nc4ccc(C5CCC6(CC5)CN(C5Cc7ccc8c(C9CCC(=O)NC9=O)noc8c7C5)C6)cc4)n3)C2)C1=O ; CCOC(=O)Cc1noc2c3c(ccc12)CC(N1CC2(CCC(c4ccc(Nc5nc(N6CCC[C@@H](N7CCN(C)C7=O)C6)cnc5C(N)=O)cc4)CC2)C1)C3 ; CN1CCN(C2CCCN(c3cnc(C(N)=O)c(Nc4ccc(C5CCC6(CC5)CN(C5Cc7ccc8c(C9CCC(=O)NC9=O)noc8c7C5)C6)cc4)n3)C2)C1=O]

### Step 1: Preparation of Compound 55

Intermediate 54a-2 (100 mg), acrylamide (15 mg) and tetrahydrofuran (5 mL) were added to a reaction flask in sequence, and the mixture was cooled to 0° C. under N₂ atmosphere. Potassium tert-butoxide (1 M, 0.079 mL) was added, and the mixture was reacted at 0° C. After the reaction was completed, as confirmed by TLC, the reaction solution was added dropwise to an icy saturated aqueous ammonium chloride solution, and 50 mL of ethyl acetate was added for extraction. The organic phase was dried over anhydrous sodium sulfate, filtered, and concentrated, and the residue was purified by silica gel column chromatography to give compound 55 (33 mg).

MS(ESI, [M+H]⁺) m/z: 787.46.

¹H NMR (500 MHz, DMSO-d6) δ 11.20 (s, 1H), 11.09 (d, J=1.5 Hz, 1H), 7.75 (d, J=2.8 Hz, 1H), 7.65 (s, 1H), 7.62 (d, J=7.4 Hz, 1H), 7.48 (d, J=8.3 Hz, 2H), 7.33 (d, J=2.8 Hz, 1H), 7.26 (d, J=8.0 Hz, 1H), 7.12 (d, J=8.2 Hz, 2H), 4.57 (dd, J=11.9, 5.0 Hz, 1H), 4.32 (dd, J=40.5, 12.7 Hz, 2H), 3.65-3.56 (m, 1H), 3.43-3.33 (m, 2H), 3.26 (dd, J=9.0, 7.3 Hz, 2H), 3.18-2.89 (m, 9H), 2.77 (ddd, J=17.1, 11.9, 5.2 Hz, 3H), 2.71 (s, 3H), 2.60 (dq, J=17.4, 4.0 Hz, 1H), 2.51-2.53 (m, 1H), 2.42-2.33 (m, 1H), 2.25-2.13 (m, 1H), 1.99-1.86 (m, 2H), 1.85-1.79 (m, 2H), 1.76 (dt, J=7.0, 3.1 Hz, 1H), 1.67 (d, J=12.1 Hz, 2H), 1.61-1.44 (m, 3H), 1.44-1.32 (m, 2H).

### Example 56: Synthesis of Compound 56

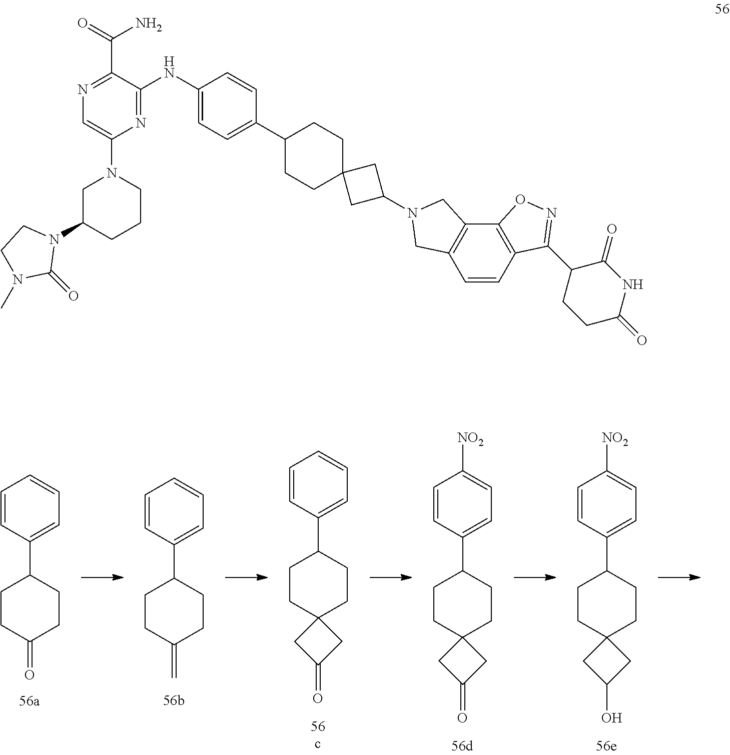

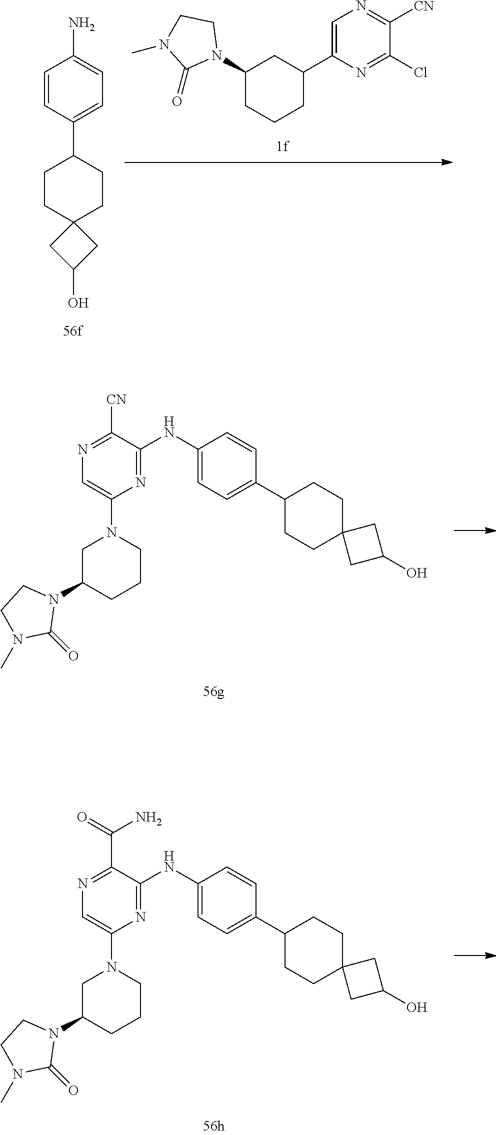

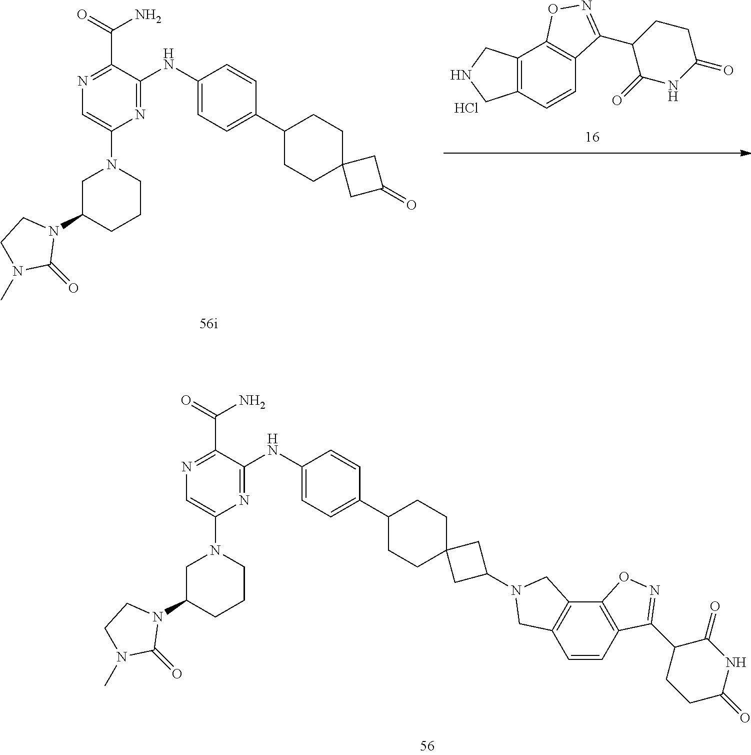 [SMILES: CN1CCN([C@@H]2CCCN(c3cnc(C(N)=O)c(Nc4ccc(C5CCC6(CC5)CC(=O)C6)cc4)n3)C2)C1=O ; Cl ; O=C1CCC(c2noc3c4c(ccc23)CNC4)C(=O)N1 ; CN1CCN([C@@H]2CCCN(c3cnc(C(N)=O)c(Nc4ccc(C5CCC6(CC5)CC(N5Cc7ccc8c(C9CCC(=O)NC9=O)noc8c7C5)C6)cc4)n3)C2)C1=O]

### Step 1: Preparation of Compound 56b

Lithium diisopropylamide (2 M, 21.52 mL) was slowly added dropwise to a stirred solution of methyl triphenyl phosphonium bromide (13.33 g, 37.3 mmol) in THF (50 mL) at 0° C. under nitrogen atmosphere, and the mixture was reacted at room temperature for 10 min 56a (5 g, 28.7 mmol) was added to the above reaction solution, and the mixture was reacted at room temperature. After the reaction was completed, 200 mL of petroleum ether and 100 mL of water were added to the reaction solution for extraction. The organic phase was dried over anhydrous sodium sulfate, filtered, and concentrated to give 56b (4.6 g).

### Step 2: Preparation of Compound 56c

56b (7.43 g), Cu—Zn (16.68 g, 129 mmol), and 100 mL of diethyl ether were added to a reaction flask in sequence. After the mixture was purged with nitrogen, trichloroacetyl chloride (9.63 mL) was added, and the mixture was reacted under an ice bath. After the reaction was completed, the reaction solution was added to about 300 mL of a saturated sodium bicarbonate solution to quench the reaction, and the mixture was slurried with 200 mL of ethyl acetate. The solid was removed by filtration under vacuum, and the mother liquor was extracted. The organic phase was dried over anhydrous sodium sulfate, filtered, and concentrated. Zinc powder (8.46 g, 129 mmol) was added to the concentrate, 30 mL of acetic acid and 15 mL of water were added, and the mixture was reacted under reflux. After the reaction was completed, the reaction solution was poured into 300 mL of a saturated sodium bicarbonate solution, and the mixture was slurried with 200 mL of petroleum ether. Filtration under vacuum was performed, and the mother liquor was extracted for liquid separation. The organic phase was dried over anhydrous sodium sulfate, filtered, and concentrated to give 56c (4.5 g).

¹H NMR (500 MHz, DMSO-4) δ 7.31-7.25 (m, 2H), 7.25-7.21 (m, 2H), 7.20-7.14 (m, 1H), 2.83 (q, J=2.7, 2.0 Hz, 2H), 2.80-2.74 (m, 2H), 2.57-2.51 (m, 1H), 1.83-1.74 (m, 4H), 1.70 (td, J=13.6, 12.6, 3.6 Hz, 2H), 1.52-1.40 (in, 2H).

### Step 3: Preparation of Intermediate 56d

56c (4.5 g), dichloromethane (50 mL), and sulfuric acid (2.251 mL) were added to a reaction flask in sequence, and nitric acid (1.62 mL) was added under an ice bath. The mixture was reacted at room temperature. After the reaction was completed, the reaction solution was added to a saturated sodium bicarbonate solution, and 200 mL of ethyl acetate was added for extraction. The organic phase was dried over anhydrous sodium sulfate, filtered, and concentrated, and the concentrate was slurried with a small amount of methyl tert-butyl ether and filtered under vacuum to give 56d (1.68 g).

¹H NMR (500 MHz, DMSO-d6) δ 7.28 (t, J=7.5 Hz, 2H), 7.25-7.21 (m, 2H), 2.82 (d, J=2.8 Hz, 2H), 2.78-2.75 (m, 2H), 2.54 (dt, J=12.2, 3.3 Hz, 1H), 1.85-1.69 (m, 8H).

### Step 4: Preparation of Intermediate 56e

56d (1.68 g), MeOH (20 mL), and sodium borohydride (0.245 g) were added to a reaction flask in sequence under an ice bath, and the mixture was reacted at room temperature. After the reaction was completed, the reaction solution was added to a saturated ammonium chloride solution, and 200 mL of ethyl acetate was added for extraction. The organic phase was dried over anhydrous sodium sulfate, filtered, and concentrated to give 56e (1.75 g).

¹H NMR (500 MHz, DMSO-d6) δ 8.21-8.12 (m, 2H), 7.55-7.47 (m, 2H), 4.87 (d, J=6.3 Hz, 1H), 4.11-4.07 (m, 1H), 2.60 (tt, J=11.7, 3.2 Hz, 1H), 2.27 (td, J=7.2, 3.6 Hz, 1H), 2.01 (dq, J=7.4, 4.1, 3.6 Hz, 1H), 1.68-1.46 (m, 10H).

### Step 5: Preparation of Intermediate 56f

56e (3.2 g), iron powder (2.5 g), ammonium chloride (3 g), 30 mL of MeOH, and 10 mL of water were added to a reaction flask in sequence, and the mixture was warmed to 80° C. and reacted. After the reaction was completed, the solid was removed by filtration under vacuum, 200 mL of ethyl acetate was added to the mother liquor, and 200 mL of a saturated sodium carbonate solution was added for extraction. The organic phase was dried over anhydrous sodium sulfate, filtered, and concentrated to give 56f (2.09 g).

MS(ESI, [M+H]⁺) m/z: 232.05.

### Step 6: Preparation of Intermediate 56g

Intermediate 1f (1.4 g), 56f (1.363 g), cesium carbonate (4.27 g), BINAP (0.272 g), palladium acetate (0.098 g), and 1,4-dioxane (10 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 56g (1.5 g).

MS(ESI, [M+H]⁺) m/z: 516.48.

### Step 7: Preparation of Intermediate 56h

Intermediate 56g (1.5 g), DMSO (5 mL), and cesium carbonate (0.948 g) were added to a single-necked flask in sequence. 30 wt % hydrogen peroxide (2 mL) was added under an ice bath. After the mixture was stirred for 5 min, the ice bath was removed, and the mixture was reacted at room temperature. After the reaction was completed, 300 mL of a saturated sodium sulfite solution was added to the residue to quench the reaction. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 56h (1.35 g).

MS(ESI, [M+H]+) m/z: 534.50.

### Step 8: Preparation of Intermediate 561

56h (1 g), IBX (1.574 g), and DMSO (15 mL) were added to a reaction flask in sequence, and the mixture was reacted at room temperature. After the reaction was completed, 200 mL of ethyl acetate was added, and 200 mL of a saturated sodium bicarbonate solution was added for extraction. The organic phase was dried over anhydrous sodium sulfate, filtered, and concentrated to give 561 (980 mg).

MS(ESI, [M+H]+) m/z: 532.49.

### Step 9: Preparation of Compound 56

56i (70 mg), isopropanol (1 mL), 1,2-dichloroethane (5.00 mL), and 16 (60.9 mg) were added to a reaction flask in sequence, and sodium cyanoborohydride (25 mg) was added. The mixture was reacted at room temperature. After the reaction was completed, the reaction solution was mixed with silica gel and purified by column chromatography to give 56 (21 mg).

MS(ESI, [M+H]⁺) m/z: 787.48.

¹H NMR (500 MHz, DMSO-d6) δ 11.20 (s, 1H), 11.10 (s, 1H), 7.79-7.74 (m, 1H), 7.72 (d, J=8.1 Hz, 1H), 7.65 (s, 1H), 7.49 (d, J=8.0 Hz, 2H), 7.38-7.27 (m, 2H), 7.14 (d, J=8.1 Hz, 2H), 4.60 (dd, J=11.9, 5.0 Hz, 1H), 4.33 (dd, J=47.2, 12.8 Hz, 2H), 4.10 (s, 2H), 4.07-3.91 (m, 2H), 3.61 (q, J=9.2, 6.4 Hz, 1H), 3.42-3.34 (m, 2H), 3.27 (d, J=7.0 Hz, 2H), 2.99 (dt, J=34.2, 11.8 Hz, 2H), 2.85-2.69 (m, 4H), 2.61 (dt, J=17.4, 4.2 Hz, 1H), 2.56-2.51 (m, 1H), 2.39 (t, J=11.0 Hz, 1H), 2.25-2.08 (m, 2H), 1.97-1.32 (m, 16H).

### Example 57: Synthesis of Compound 57

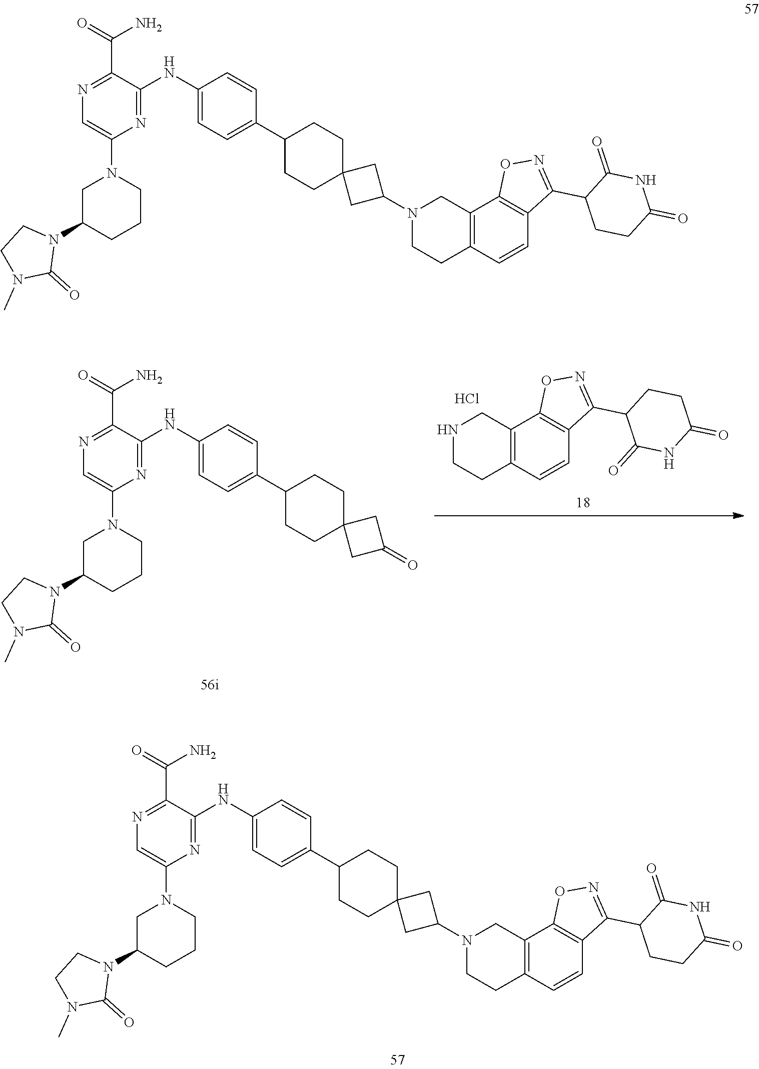 [SMILES: CN1CCN([C@@H]2CCCN(c3cnc(C(N)=O)c(Nc4ccc(C5CCC6(CC5)CC(N5CCc7ccc8c(C9CCC(=O)NC9=O)noc8c7C5)C6)cc4)n3)C2)C1=O ; CN1CCN([C@@H]2CCCN(c3cnc(C(N)=O)c(Nc4ccc(C5CCC6(CC5)CC(=O)C6)cc4)n3)C2)C1=O ; O=C1CCC(c2noc3c4c(ccc23)CCNC4)C(=O)N1 ; Cl ; CN1CCN([C@@H]2CCCN(c3cnc(C(N)=O)c(Nc4ccc(C5CCC6(CC5)CC(N5CCc7ccc8c(C9CCC(=O)NC9=O)noc8c7C5)C6)cc4)n3)C2)C1=O]

### Step 1: Preparation of Compound 57

56i (70 mg), isopropanol (1 mL), 1,2-dichloroethane (5.00 mL), and 18 (50 mg) were added to a reaction flask in sequence, and sodium cyanoborohydride (25 mg) was added. The mixture was reacted at room temperature. After the reaction was completed, the reaction solution was mixed with silica gel and purified by column chromatography to give 57 (28 mg).

MS(ESI, [M+H]+) m/z: 801.49.

¹H NMR (500 MHz, DMSO-d6) δ 11.20 (d, J=2.3 Hz, 1H), 11.09 (s, 1H), 7.75 (d, J=2.8 Hz, 1H), 7.65 (s, 1H), 7.59 (d, J=8.1 Hz, 1H), 7.49 (d, J=8.2 Hz, 2H), 7.33 (d, J=2.8 Hz, 1H), 7.14 (dd, J=8.4, 3.7 Hz, 3H), 4.56 (dd, J=11.9, 5.0 Hz, 1H), 4.33 (dd, J=44.6, 13.0 Hz, 2H), 3.69 (s, 2H), 3.61 (t, J=7.6 Hz, 1H), 3.39-3.32 (m, 2H), 3.28 (qd, J=8.0, 4.1 Hz, 2H), 3.06-2.90 (m, 5H), 2.82-2.70 (m, 4H), 2.67-2.57 (m, 3H), 2.44-2.35 (m, 1H), 2.25-2.15 (m, 2H), 2.01-1.94 (m, 1H), 1.91-1.32 (in, 15H).

### Example 58: Synthesis of Compound 58

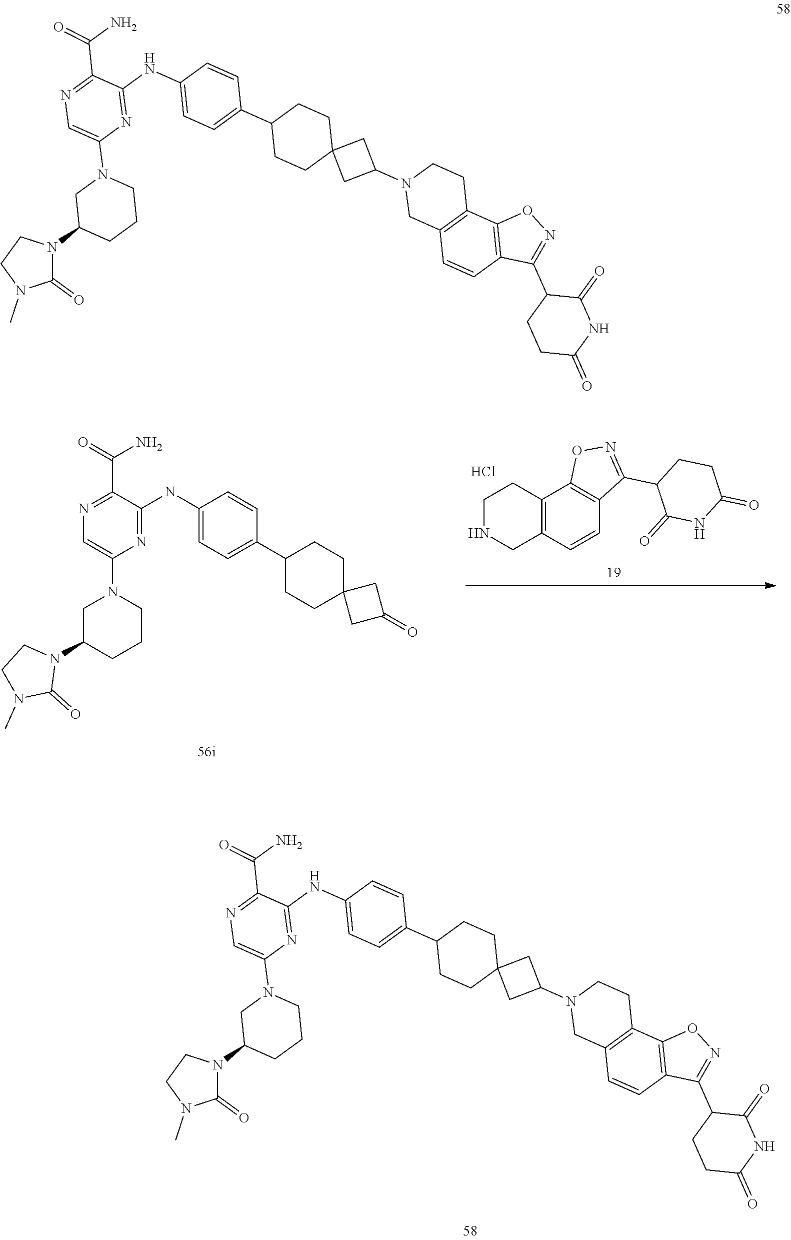

### Step 1: Preparation of Compound 58

56i (70 mg), isopropanol (1 mL), 1,2-dichloroethane (5.00 mL), and 19 (50 mg) were added to a reaction flask in sequence, and sodium cyanoborohydride (25 mg) was added. The mixture was reacted at room temperature. After the reaction was completed, the reaction solution was mixed with silica gel and purified by column chromatography to give 58 (15 mg).

MS(ESI, [M+H]+) m/z: 801.47.

¹H NMR (500 MHz, DMSO-d6) δ 11.28-11.14 (m, 1H), 11.09 (s, 1H), 7.75 (d, J=2.9 Hz, 1H), 7.65 (s, 1H), 7.58 (d, J=8.0 Hz, 1H), 7.49 (d, J=8.1 Hz, 2H), 7.33 (d, J=3.0 Hz, 1H), 7.14 (d, J=8.4 Hz, 3H), 4.56 (dd, J=11.9, 5.1 Hz, 1H), 4.37 (d, J=12.6 Hz, 1H), 4.29 (d, J=13.2 Hz, 1H), 3.68-3.51 (m, 3H), 3.45-3.24 (m, 6H), 3.15-2.84 (m, 5H), 2.80-2.56 (m, 6H), 2.38 (d, J=14.4 Hz, 1H), 2.19 (dq, J=15.9, 7.2, 5.9 Hz, 2H), 1.95 (s, 1H), 1.87-1.33 (m, 14H).

### Example 59: Synthesis of Compound 59

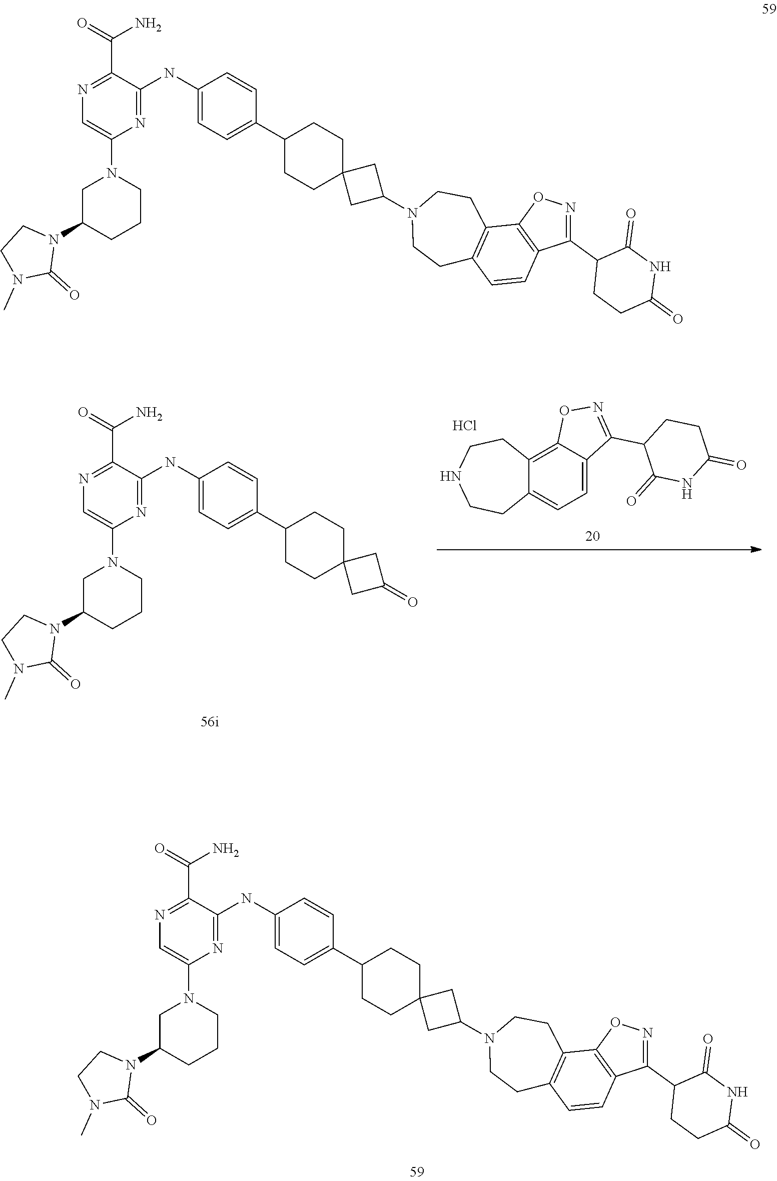

### Step 1: Preparation of Compound 59

56i (70 mg), isopropanol (1 mL), 1,2-dichloroethane (5.00 mL), and 20 (53 mg) were added to a reaction flask in sequence, and sodium cyanoborohydride (25 mg) was added. The mixture was reacted at room temperature. After the reaction was completed, the reaction solution was mixed with silica gel and purified by column chromatography to give 59 (24 mg).

MS(ESI, [M+H]+) m/z: 815.42.

¹H NMR (500 MHz, DMSO-d6) δ 11.20 (d, J=2.4 Hz, 1H), 11.08 (s, 1H), 7.75 (d, J=2.9 Hz, 1H), 7.65 (s, 1H), 7.54 (d, J=8.0 Hz, 1H), 7.49 (d, J=8.2 Hz, 2H), 7.33 (d, J=2.9 Hz, 1H), 7.19 (d, J=8.1 Hz, 1H), 7.13 (d, J=8.2 Hz, 2H), 4.55 (dd, J=11.9, 5.0 Hz, 1H), 4.47-4.23 (m, 2H), 3.62 (ddt, J=11.3, 8.6, 4.0 Hz, 1H), 3.37 (d, J=8.3 Hz, 2H), 3.31-3.24 (m, 2H), 3.19-3.11 (m, 2H), 3.09-2.92 (m, 4H), 2.84-2.71 (m, 5H), 2.60 (dt, J=17.3, 4.2 Hz, 1H), 2.49-2.33 (m, 5H), 2.22-2.10 (m, 2H), 1.93-1.38 (m, 16H).

### Example 60: Synthesis of Compound 60

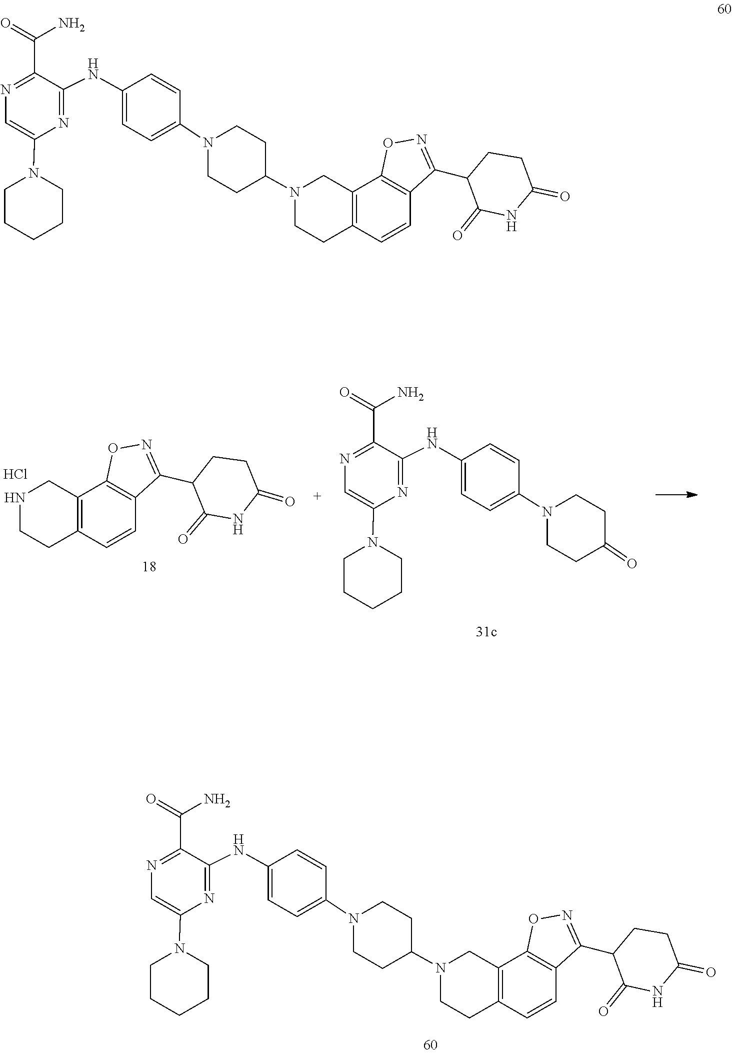 [SMILES: NC(=O)c1ncc(N2CCCCC2)nc1Nc1ccc(N2CCC(N3CCc4ccc5c(C6CCC(=O)NC6=O)noc5c4C3)CC2)cc1 ; Cl ; O=C1CCC(c2noc3c4c(ccc23)CCNC4)C(=O)N1 ; NC(=O)c1ncc(N2CCCCC2)nc1Nc1ccc(N2CCC(=O)CC2)cc1 ; NC(=O)c1ncc(N2CCCCC2)nc1Nc1ccc(N2CCC(N3CCc4ccc5c(C6CCC(=O)NC6=O)noc5c4C3)CC2)cc1]

### Step 1: Preparation of Compound 60

31c (100 mg), isopropanol (1 mL), 1,2-dichloroethane (5.00 mL), and 18 (82 mg) were added to a reaction flask in sequence, and sodium cyanoborohydride (31 mg) was added. The mixture was reacted at room temperature. After the reaction was completed, the reaction solution was mixed with silica gel and purified by column chromatography to give 60 (45 mg).

MS(ESI, [M+H]+) m/z: 664.4.

¹H NMR (500 MHz, DMSO-d6) δ 11.08 (d, J=6.4 Hz, 2H), 7.69 (d, J=2.9 Hz, 1H), 7.60 (s, 1H), 7.58 (d, J=8.1 Hz, 1H), 7.48-7.39 (m, 2H), 7.26 (d, J=3.0 Hz, 1H), 7.14 (d, J=8.2 Hz, 1H), 6.97-6.89 (m, 2H), 4.56 (dd, J=11.9, 5.0 Hz, 1H), 3.98 (s, 2H), 3.70 (d, J=11.7 Hz, 2H), 3.65 (t, J=5.4 Hz, 4H), 2.94 (t, J=5.4 Hz, 2H), 2.87 (t, J=5.6 Hz, 2H), 2.76 (td, J=12.0, 5.9 Hz, 1H), 2.72-2.57 (m, 4H), 2.48 (d, J=4.1 Hz, 1H), 2.24-2.13 (m, 1H), 1.97 (d, J=11.7 Hz, 2H), 1.68 (ddt, J=22.6, 11.1, 4.6 Hz, 4H), 1.58 (h, J=5.5, 4.4 Hz, 4H).

### Example 61: Synthesis of Compound

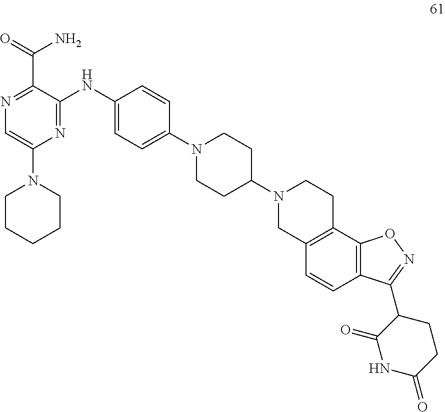 [SMILES: NC(=O)c1ncc(N2CCCCC2)nc1Nc1ccc(N2CCC(N3CCc4c(ccc5c(C6CCC(=O)NC6=O)noc45)C3)CC2)cc1]

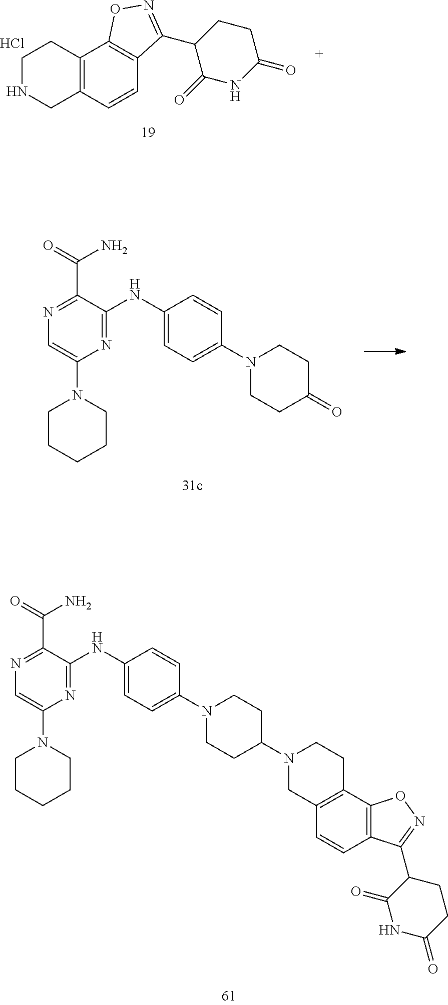 [SMILES: Cl ; O=C1CCC(c2noc3c4c(ccc23)CNCC4)C(=O)N1 ; NC(=O)c1ncc(N2CCCCC2)nc1Nc1ccc(N2CCC(=O)CC2)cc1 ; NC(=O)c1ncc(N2CCCCC2)nc1Nc1ccc(N2CCC(N3CCc4c(ccc5c(C6CCC(=O)NC6=O)noc45)C3)CC2)cc1]

### Step 1: Preparation of Compound 61

31c (100 mg), isopropanol (1 mL), 1,2-dichloroethane (5.00 mL), and 19 (82 mg) were added to a reaction flask in sequence, and sodium cyanoborohydride (31 mg) was added. The mixture was reacted at room temperature. After the reaction was completed, the reaction solution was mixed with silica gel and purified by column chromatography to give 61 (41 mg).

MS(ESI, [M+H]+) m/z: 664.78.

¹H NMR (500 MHz, DMSO-d6) δ 11.08 (d, J=4.3 Hz, 2H), 7.69 (d, J=3.0 Hz, 1H), 7.61 (s, 1H), 7.57 (d, J=8.2 Hz, 1H), 7.44 (d, J=8.7 Hz, 2H), 7.26 (d, J=2.9 Hz, 1H), 7.12 (d, J=8.3 Hz, 1H), 6.93 (d, J=8.7 Hz, 2H), 4.55 (dd, J=11.9, 5.0 Hz, 1H), 3.89 (s, 2H), 3.70 (d, J=11.7 Hz, 2H), 3.66 (t, J=5.3 Hz, 4H), 2.97 (d, J=5.7 Hz, 2H), 2.90 (t, J=5.7 Hz, 2H), 2.77 (ddd, J=17.2, 11.9, 5.3 Hz, 1H), 2.71-2.56 (m, 4H), 2.49-2.43 (m, 1H), 2.19 (dt, J=13.4, 4.6 Hz, 1H), 1.97-1.88 (m, 2H), 1.66 (td, J=12.0, 4.5 Hz, 4H), 1.58 (q, J=5.7, 5.2 Hz, 4H).

### Example 62: Synthesis of Compound 62

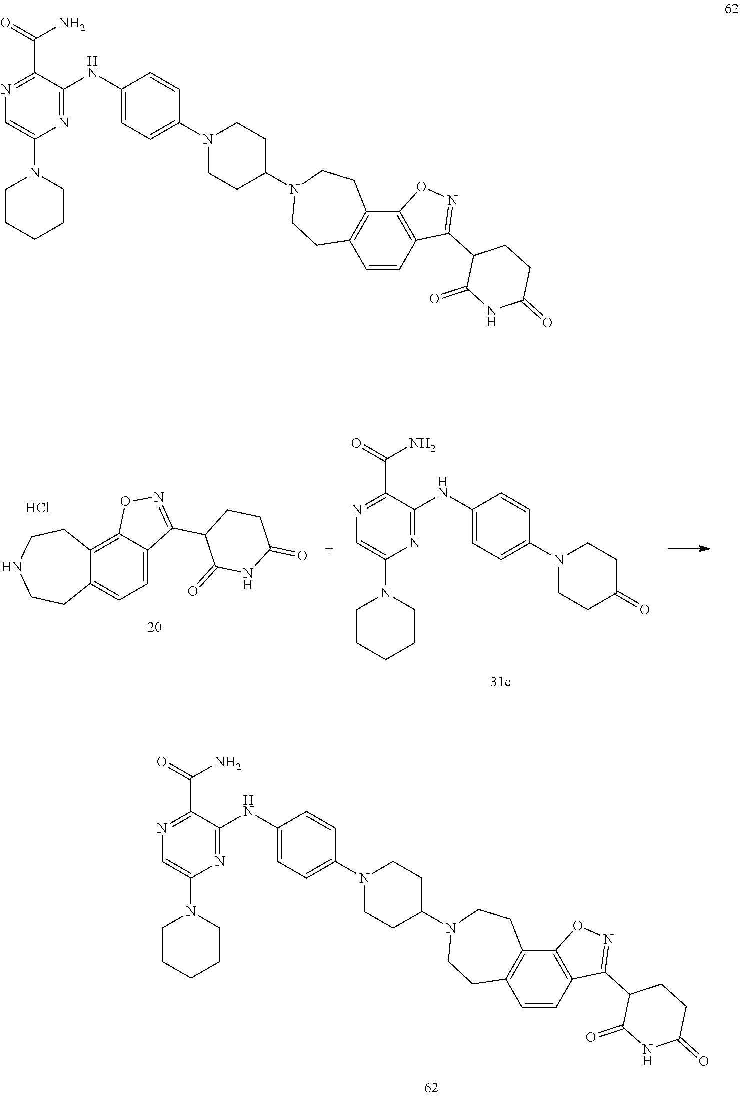 [SMILES: NC(=O)c1ncc(N2CCCCC2)nc1Nc1ccc(N2CCC(N3CCc4ccc5c(C6CCC(=O)NC6=O)noc5c4CC3)CC2)cc1 ; O=C1CCC(c2noc3c4c(ccc23)CCNCC4)C(=O)N1 ; Cl ; NC(=O)c1ncc(N2CCCCC2)nc1Nc1ccc(N2CCC(=O)CC2)cc1 ; NC(=O)c1ncc(N2CCCCC2)nc1Nc1ccc(N2CCC(N3CCc4ccc5c(C6CCC(=O)NC6=O)noc5c4CC3)CC2)cc1]

### Step 1: Preparation of Compound 62

31c (100 mg), isopropanol (1 mL), 1,2-dichloroethane (5.00 mL), and 20 (85 mg) were added to a reaction flask in sequence, and sodium cyanoborohydride (31 mg) was added. The mixture was reacted at 80° C. After the reaction was completed, the reaction solution was mixed with silica gel and purified by column chromatography to give 62 (23 mg).

MS(ESI, [M+H]+) m/z: 678.81.

¹H NMR (500 MHz, DMSO-d6) δ 11.07 (d, J=13.3 Hz, 2H), 7.69 (s, 1H), 7.60 (s, 1H), 7.54 (d, J=8.0 Hz, 1H), 7.42 (d, J=8.4 Hz, 2H), 7.26 (s, 1H), 7.19 (d, J=8.1 Hz, 1H), 6.90 (d, J=8.4 Hz, 2H), 4.55 (dd, J=11.9, 4.9 Hz, 1H), 3.66 (q, J=7.1, 5.2 Hz, 6H), 3.14 (d, J=5.2 Hz, 2H), 3.08-2.97 (m, 2H), 2.86-2.70 (m, 5H), 2.68-2.56 (m, 5H), 2.18 (dd, J=13.3, 5.8 Hz, 1H), 1.76 (d, J=11.9 Hz, 2H), 1.69-1.48 (m, 8H).

### Examples 63 and 64: Synthesis of Compounds 63 and 64

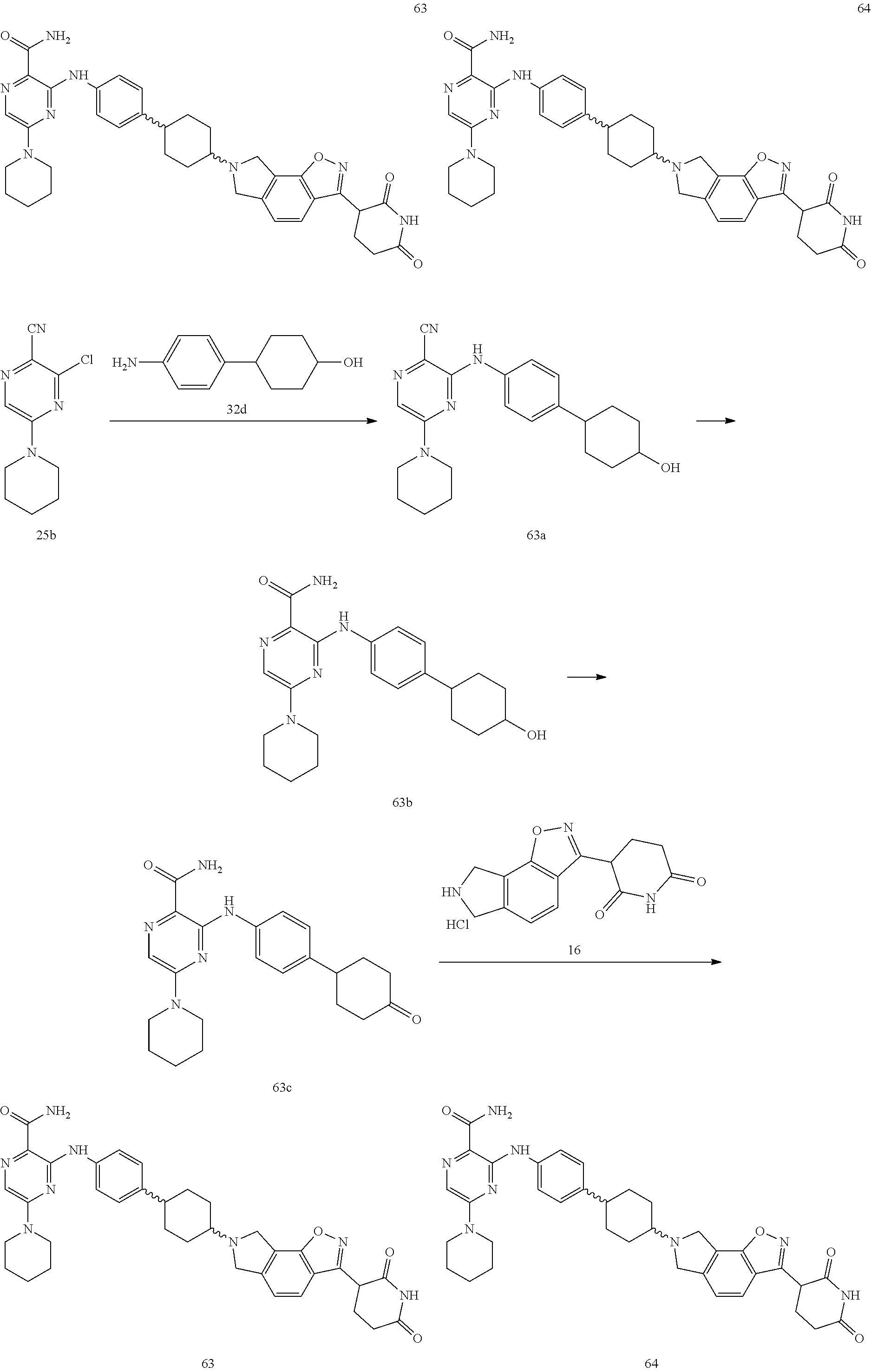

### Step 1: Preparation of Intermediate 63a

Intermediate 25b (2 g), 32d (2.58 g), cesium carbonate (5.85 g), BINAP (0.559 g), palladium acetate (0.202 g), and 1,4-dioxane (20 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 63a (3.2 g).

MS(ESI, [M+H]+) m/z: 378.25.

### Step 2: Preparation of Intermediate 63b

Intermediate 63a (3.2 g), DMSO (30 mL), and cesium carbonate (2.76 g) were added to a single-necked flask in sequence. 30 wt % hydrogen peroxide (2.88 mL) was added under an ice bath. After the mixture was stirred for 5 min, the ice bath was removed, and the mixture was reacted at room temperature. After the reaction was completed, 300 mL of a saturated sodium sulfite solution was added to the residue. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 63b (3.3 g).

MS(ESI, [M+H]+) m/z: 396.18.

### Step 3: Preparation of Intermediate 63c

63b (3.3 g), IBX (7.01 g), and DMSO (30 mL) were added to a reaction flask in sequence, and the mixture was reacted at room temperature. After the reaction was completed, 200 mL of ethyl acetate was added, and 200 mL of a saturated sodium bicarbonate solution was added for extraction. The organic phase was dried over anhydrous sodium sulfate, filtered, and concentrated to give 63c (770 mg).

MS(ESI, [M+H]+) m/z: 394.21.

### Step 4: Preparation of Compounds 63 and 64

63c (0.22 g), isopropanol (1 mL), 1,2-dichloroethane (5.00 mL), and 16 (0.172 g) were added to a reaction flask in sequence, and sodium cyanoborohydride (0.105 g) was added. The mixture was reacted at room temperature. After the reaction was completed, the reaction solution was mixed with silica gel and purified by column chromatography (ratio of developing solvents: MeOH:DCM=0:100-1:25) to give 63 (32 mg) and 64 (82 mg) in sequence.

63: MS(ESI, [M+H]+) m/z: 649.55.

¹H NMR (500 MHz, DMSO-d6) δ 11.28 (s, 1H), 11.10 (s, 1H), 8.01-7.60 (m, 3H), 7.52 (d, J=8.4 Hz, 2H), 7.37-7.25 (m, 2H), 7.20 (d, J=8.2 Hz, 2H), 4.60 (dd, J=11.9, 5.0 Hz, 1H), 4.16 (d, J=53.1 Hz, 4H), 3.68 (t, J=5.4 Hz, 4H), 2.77 (td, J=12.0, 5.9 Hz, 1H), 2.67-2.51 (m, 4H), 2.28-2.09 (m, 3H), 1.88 (d, J=12.4 Hz, 2H), 1.73-1.33 (m, 10H).

64: MS(ESI, [M+H]+) m/z: 649.53.

¹H NMR (500 MHz, DMSO-d6) δ 11.26 (s, 1H), 11.10 (s, 1H), 7.73 (d, J=8.4 Hz, 2H), 7.64 (d, J=4.9 Hz, 1H), 7.48 (d, J=7.9 Hz, 2H), 7.38-7.22 (m, 2H), 7.15 (d, J=8.0 Hz, 2H), 4.61 (dd, J=11.7, 4.9 Hz, 1H), 4.18 (s, 2H), 4.08 (d, J=5.2 Hz, 2H), 3.73-3.56 (m, 4H), 2.91-2.70 (m, 2H), 2.60 (ddd, J=31.6, 17.5, 8.7 Hz, 3H), 2.32-2.18 (m, 1H), 2.06 (d, J=13.3 Hz, 2H), 1.87 (p, J=12.2, 9.0 Hz, 2H), 1.76-1.43 (m, 10H).

### Examples 65 and 66: Synthesis of Compounds 65 and 66

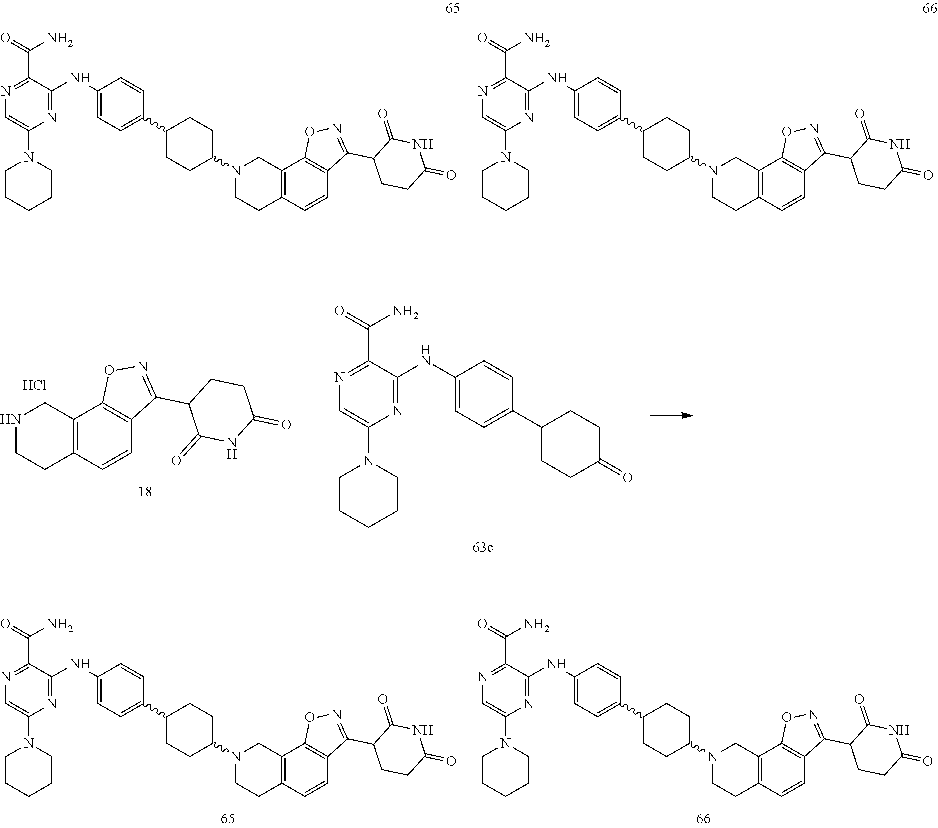

### Step 1: Preparation of Compounds 65 and 66

63c (150 mg), isopropanol (1 mL), 1,2-dichloroethane (5.00 mL), and 18 (123 mg) were added to a reaction flask in sequence, and sodium cyanoborohydride (71.9 mg) was added. The mixture was reacted at room temperature. After the reaction was completed, the reaction solution was mixed with silica gel and purified by column chromatography (ratio of developing solvents: MeOH:DCM=0:100-1:25) to give 65 (33 mg) and 66 (29 mg) in sequence.

65: MS(ESI, [M+H]+) m/z: 663.34.

¹H NMR (500 MHz, DMSO-d6) δ 11.25 (s, 1H), 11.08 (s, 1H), 7.71 (d, J=2.8 Hz, 1H), 7.64 (s, 1H), 7.60 (d, J=8.1 Hz, 1H), 7.48 (d, J=8.2 Hz, 2H), 7.29 (d, J=2.8 Hz, 1H), 7.16 (d, J=8.2 Hz, 3H), 4.56 (dd, J=11.9, 5.0 Hz, 1H), 3.91 (s, 2H), 3.65 (t, J=5.4 Hz, 4H), 2.97 (s, 2H), 2.84 (t, J=5.5 Hz, 2H), 2.77 (ddd, J=17.2, 12.0, 5.3 Hz, 1H), 2.70-2.60 (m, 2H), 2.60-2.53 (m, 1H), 2.49-2.44 (m, 1H), 2.23-2.09 (m, 3H), 1.87 (q, J=14.1, 11.7 Hz, 2H), 1.70-1.50 (m, 10H).

66: MS(ESI, [M+H]+) m/z: 663.56.

¹H NMR (500 MHz, DMSO-d6) δ 11.28 (s, 1H), 11.09 (s, 1H), 7.73 (d, J=2.8 Hz, 1H), 7.66 (s, 1H), 7.58 (d, J=8.1 Hz, 1H), 7.55-7.49 (m, 2H), 7.31 (d, J=2.8 Hz, 1H), 7.24-7.16 (m, 2H), 7.14 (d, J=8.2 Hz, 1H), 4.56 (dd, J=11.9, 5.0 Hz, 1H), 3.99 (s, 2H), 3.68 (t, J=5.4 Hz, 4H), 2.94 (d, J=5.7 Hz, 2H), 2.88 (t, J=5.5 Hz, 2H), 2.77 (ddd, J=17.2, 12.0, 5.3 Hz, 1H), 2.71-2.56 (m, 2H), 2.53 (s, 1H), 2.47 (dd, J=12.3, 4.3 Hz, 1H), 2.19 (dq, J=13.6, 4.8 Hz, 1H), 2.00 (s, 2H), 1.91 (d, J=7.3 Hz, 2H), 1.72-1.48 (m, 10H).

### Examples 67 and 68: Synthesis of Compounds 67 and 68

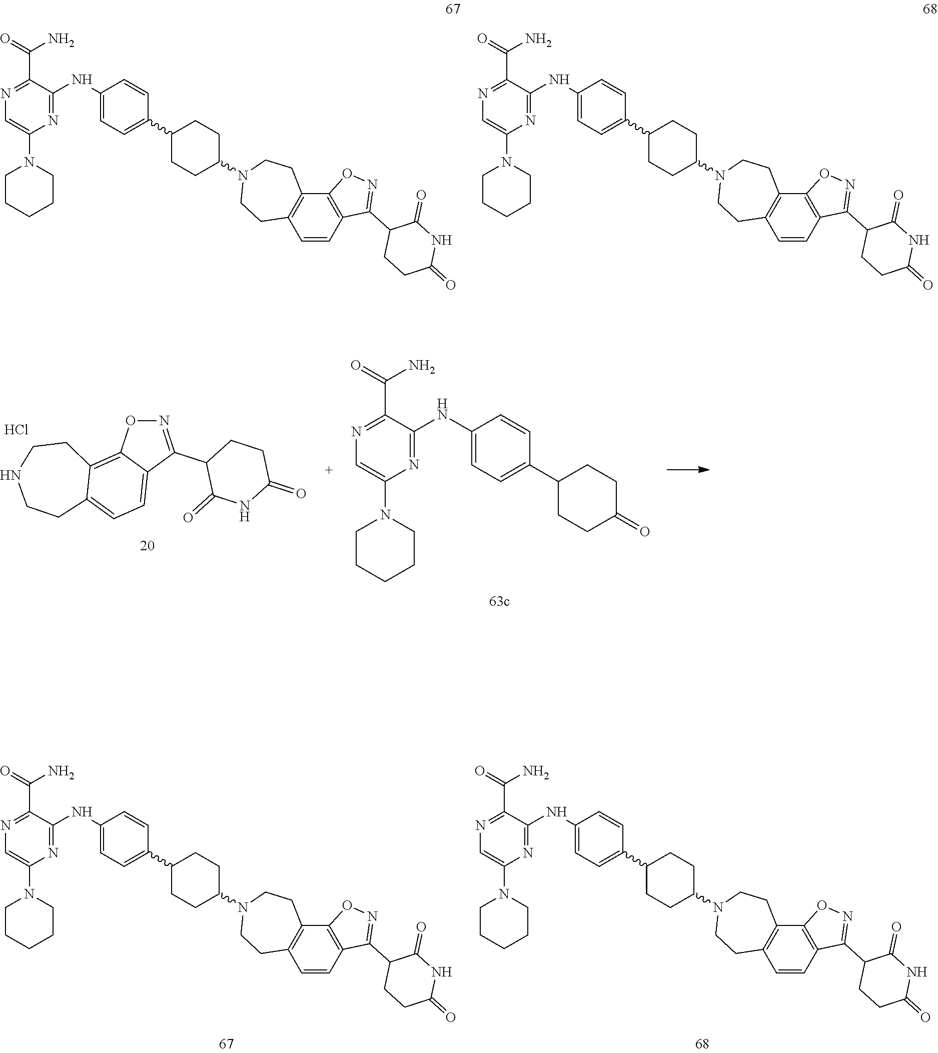

### Step 1: Preparation of Compounds 67 and 68

63c (200 mg), isopropanol (1 mL), 1,2-dichloroethane (5.00 mL), and 20 (171 mg) were added to a reaction flask in sequence, and sodium cyanoborohydride (96 mg) was added. The mixture was reacted at 80° C. After the reaction was completed, the reaction solution was mixed with silica gel and purified by column chromatography (ratio of developing solvents: MeOH:DCM=0:100-˜ 1:25) to give 67 (18 mg) and 68 (42 mg) in sequence. 67: MS(ESI, [M+H]+) m/z: 677.52.

¹H NMR (500 MHz, DMSO-d6) δ 11.27 (s, 1H), 11.08 (s, 1H), 7.73 (d, J=2.8 Hz, 1H), 7.65 (s, 1H), 7.54 (d, J=8.0 Hz, 1H), 7.50 (d, J=8.3 Hz, 2H), 7.31 (d, J=2.8 Hz, 1H), 7.17 (dd, J=15.3, 8.2 Hz, 3H), 5.76 (s, 1H), 4.55 (dd, J=11.9, 5.0 Hz, 1H), 3.67 (t, J=5.4 Hz, 4H), 3.19-3.10 (m, 2H), 3.08-2.98 (m, 2H), 2.76 (dtd, J=20.0, 11.1, 10.1, 4.9 Hz, 4H), 2.61 (dt, J=17.3, 4.3 Hz, 2H), 2.53 (d, J=4.6 Hz, 1H), 2.40 (d, J=3.3 Hz, 1H), 2.18 (dq, J=13.5, 4.8 Hz, 1H), 1.82 (d, J=11.5 Hz, 4H), 1.62 (dq, J=33.7, 5.9, 5.4 Hz, 6H), 1.46 (q, J=10.2 Hz, 4H).

68: MS(ESI, [M+H]+) m/z: 677.49.

¹H NMR (500 MHz, DMSO-4) δ 11.27 (s, 1H), 11.08 (s, 1H), 7.73 (d, J=2.8 Hz, 1H), 7.66 (s, 1H), 7.52 (dd, J=−8.3, 2.0 Hz, 3H), 7.31 (d, J=2.9 Hz, 1H), 7.20 (d, J=8.3 Hz, 2H), 7.15 (d, J=8.1 Hz, 1H), 4.53 (dd, J=11.9, 5.0 Hz, 1H), 3.67 (t, J=5.4 Hz, 4H), 3.21-3.10 (m, 2H), 3.03 (d, J=8.1 Hz, 2H), 2.94-2.68 (m, 7H), 2.60 (dt, J=17.3, 4.2 Hz, 1H), 2.46 (dd, J=12.2, 4.3 Hz, 1H), 2.18 (dt, J=13.3, 4.6 Hz, 1H), 2.01-1.79 (m, 4H), 1.60 (ddt, J=22.5, 16.9, 8.6 Hz, 10H).

### Example 69: Synthesis of Compound 69

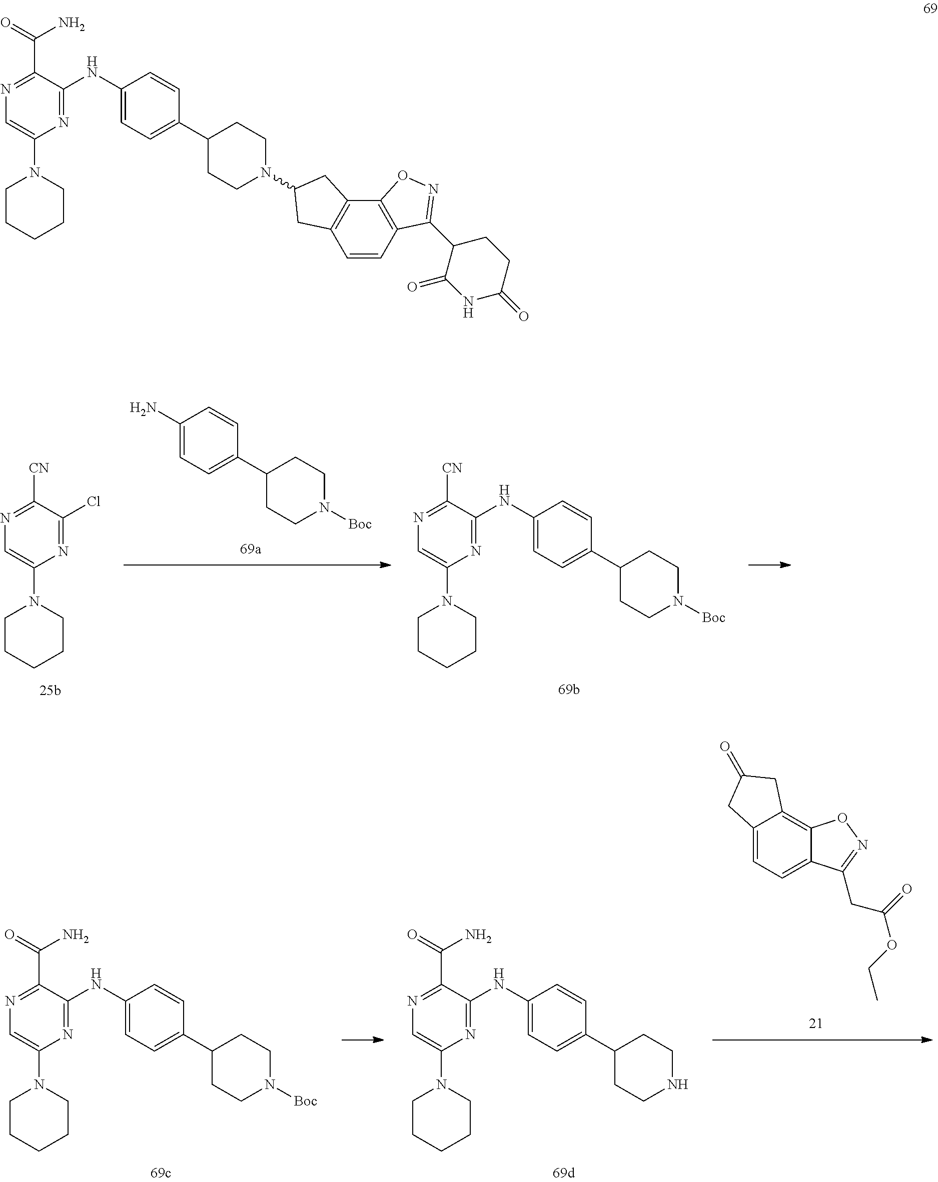

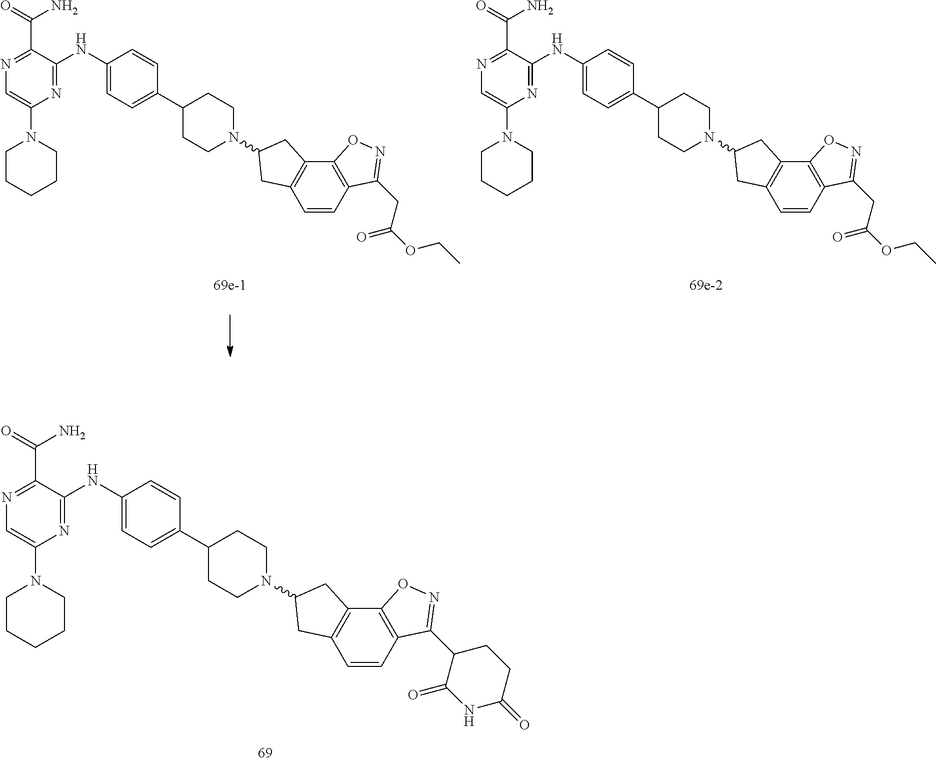 [SMILES: CCOC(=O)Cc1noc2c3c(ccc12)CC(N1CCC(c2ccc(Nc4nc(N5CCCCC5)cnc4C(N)=O)cc2)CC1)C3 ; CCOC(=O)Cc1noc2c3c(ccc12)CC(N1CCC(c2ccc(Nc4nc(N5CCCCC5)cnc4C(N)=O)cc2)CC1)C3 ; NC(=O)c1ncc(N2CCCCC2)nc1Nc1ccc(C2CCN(C3Cc4ccc5c(C6CCC(=O)NC6=O)noc5c4C3)CC2)cc1]

### Step 1: Preparation of Intermediate 69b

Intermediate 25b (4.77 g), intermediate 69a (5.92 g), cesium carbonate (20.94 g), palladium acetate (0.481 g, 2.14 mmol), BINAP (1.334 g), and 1,4-dioxane (100 mL) were added to a reaction flask in sequence, and the mixture was heated at 100° C. under nitrogen atmosphere. After the reaction was completed, 200 mL of water was added to the reaction solution, and 200 mL of ethyl acetate was added for extraction twice. The organic phases were combined and washed with saturated brine once, and the extract was dried over anhydrous sodium sulfate, filtered, subjected to rotary evaporation, mixed with silica gel, and purified by column chromatography to give the target intermediate 69b (5.53 g).

¹H NMR (500 MHz, DMSO-d6) δ 8.91 (s, 1H), 7.82 (s, 1H), 7.50-7.44 (m, 2H), 7.18-7.12 (m, 2H), 4.14-4.02 (m, 2H), 3.62 (t, J=5.4 Hz, 4H), 2.79 (s, 2H), 2.63 (tt, J=12.1, 3.5 Hz, 1H), 1.78-1.70 (m, 2H), 1.63 (q, J=6.4 Hz, 2H), 1.54 (dt, J=9.6, 5.4 Hz, 4H), 1.46 (dd, J=12.5, 4.1 Hz, 2H), 1.42 (s, 9H).

### Step 3: Preparation of Intermediate 69c

Intermediate 69b (5.40 g), cesium carbonate (3.60 g), dimethyl sulfoxide (20 mL), and methanol (40 mL) were added to a reaction flask in sequence, and 30 wt % hydrogen peroxide (1.305 g, 1.305 mL, 11.51 mmol) was added under an ice bath. The mixture was stirred and reacted at room temperature. After the reaction was completed, 100 mL of a sodium thiosulfate solution was added to the reaction solution. The reaction solution was subjected to rotary evaporation, water (100 mL) was added to the residue, and 200 mL of dichloromethane was added for extraction twice. The organic phases were combined and washed with saturated brine, the extract was dried over anhydrous sodium sulfate, filtered, and subjected to rotary evaporation to dryness, thus giving the target intermediate 69c (5.60 g).

¹H NMR (500 MHz, DMSO-d6) δ 11.29 (s, 1H), 7.78-7.71 (m, 1H), 7.66 (s, 1H), 7.52 (d, J=8.1 Hz, 2H), 7.32 (s, 1H), 7.18 (d, J=8.1 Hz, 2H), 4.04 (d, J=8.0 Hz, 2H), 3.67 (t, J=5.3 Hz, 4H), 2.79 (s, 2H), 2.66-2.59 (m, 1H), 1.74 (d, J=12.8 Hz, 2H), 1.66 (q, J=5.6 Hz, 2H), 1.62-1.55 (m, 4H), 1.51-1.44 (m, 2H), 1.42 (s, 9H).

### Step 4: Preparation of Intermediate 69d

Intermediate 69c (5.55 g) and dichloromethane (30 mL) were added to a reaction flask, and trifluoroacetic acid (35.87 g, 24.07 mL, 314.6 mmol) was added dropwise under an ice-water bath. After the dropwise addition was completed, the mixture was stirred at room temperature. After the reaction was completed, the reaction solution was subjected to rotary evaporation to remove most trifluoroacetic acid, 100 mL of water was added to the residue, a saturated sodium carbonate solution was added until no obvious bubbles were present, and the mixture was extracted 3-4 times with 200 mL of a mixed solvent of dichloromethane and methanol (10:1, v/v). The organic phases were combined, and the extract was dried over anhydrous sodium sulfate, filtered, and subjected to rotary evaporation to dryness, thus giving the target intermediate 69d (4.30 g).

¹H NMR (500 MHz, DMSO-d6) δ 11.32 (s, 1H), 7.77-7.73 (m, 1H), 7.68 (s, 1H), 7.56 (d, J=8.1 Hz, 2H), 7.35-7.31 (m, 1H), 7.17 (d, J=8.1 Hz, 2H), 3.68 (t, J=5.4 Hz, 4H), 3.36 (s, 2H), 2.97 (td, J=12.8, 2.9 Hz, 2H), 2.78 (dt, J=24.5, 3.6 Hz, 1H), 1.92 (d, J=13.5 Hz, 2H), 1.81-1.53 (m, 9H).

### Step 5: Preparation of Intermediates 69e-1 and 69e-2

Intermediate 21 (167 mg), intermediate 69d (245 mg), 1,2-dichloroethane (10 mL), isopropanol (3 mL), acetic acid (38.7 mg, 0.644 mmol), and sodium cyanoborohydride (121 mg, 1.932 mmol) were added to a reaction flask in sequence, and the mixture was reacted overnight at room temperature. After the reaction was completed, 5 mL of a saturated sodium bicarbonate solution was added to the reaction solution to neutralize acetic acid, and then 100 mL of dichloromethane and 100 mL of water were added for extraction. The organic phase was separated, and the aqueous phase was extracted twice with 50 mL of dichloromethane. The organic phases were combined, washed with saturated brine, dried over anhydrous sodium sulfate, and filtered, and the filtrate was purified by silica gel column chromatography (dichloromethane:MeOH) to give the target intermediate 691 (211 mg). Intermediate 69i was separated by high performance liquid chromatography to give intermediates 69e-1 (54 mg) and 69e-2 (56 mg) in sequence. The conditions of preparative chromatography were as follows:

Instrument and preparative column: YMC K-prep Lab100g high pressure preparative chromatograph was used, and the preparative column was Whelk-Ol (10 μm, 30*250 mm). Mobile phase system:ethanol-dichloromethane (3:2)/n-hexane-0.1% diethylamine, isocratic elution: ethanol-dichloromethane (3:2)/n-hexane-0.1% diethylamine=50/50.

Intermediate 69e-1: MS(ESI, [M+H]+) m/z: 624.55.

Intermediate 69e-2: MS(ESI, [M+H]+) m/z: 624.54.

### Step 7: Preparation of Compound 69

Intermediate 69e-1 (54 mg), acrylamide (6.15 mg, 0.087 mmol), and tetrahydrofuran (5 mL) were added to a reaction flask in sequence, and the mixture was cooled to 0° C. under N₂ atmosphere. Potassium tert-butoxide (1 mol/L, 0.061 mL, 0.061 mmol) was added, and the mixture was reacted at 0° C. for 1 h. After the reaction was completed, the reaction solution was added dropwise to an icy saturated aqueous ammonium chloride solution, and 50 mL of ethyl acetate was added for extraction. The organic phase was separated, and the aqueous phase was extracted twice with 20 mL of ethyl acetate. The organic phases were combined, washed with saturated brine, dried over anhydrous sodium sulfate, and filtered, and the filtrate was purified by silica gel column chromatography (dichloromethane:MeOH=0-4%) to give compound 69 (14 mg).

MS(ESI, [M+H]+) m/z: 649.62.

¹H NMR (500 MHz, DMSO-d6) δ 11.28 (s, 1H), 11.09 (s, 1H), 7.73 (s, 1H), 7.69-7.59 (m, 2H), 7.52 (d, J=8.0 Hz, 2H), 7.35-7.24 (m, 2H), 7.19 (d, J=8.1 Hz, 2H), 4.57 (d, J=11.7 Hz, 1H), 3.67 (s, 4H), 3.46-3.38 (m, 1H), 3.29 (s, 1H), 3.21 (d, J=16.1 Hz, 1H), 3.02 (t, J=11.8 Hz, 4H), 2.76 (d, J=14.0 Hz, 1H), 2.60 (d, J=19.9 Hz, 1H), 2.16 (dd, J=24.4, 12.4 Hz, 3H), 1.70 (dd, J=76.6, 23.6 Hz, 11H).

### Example 70: Synthesis of Compound 70

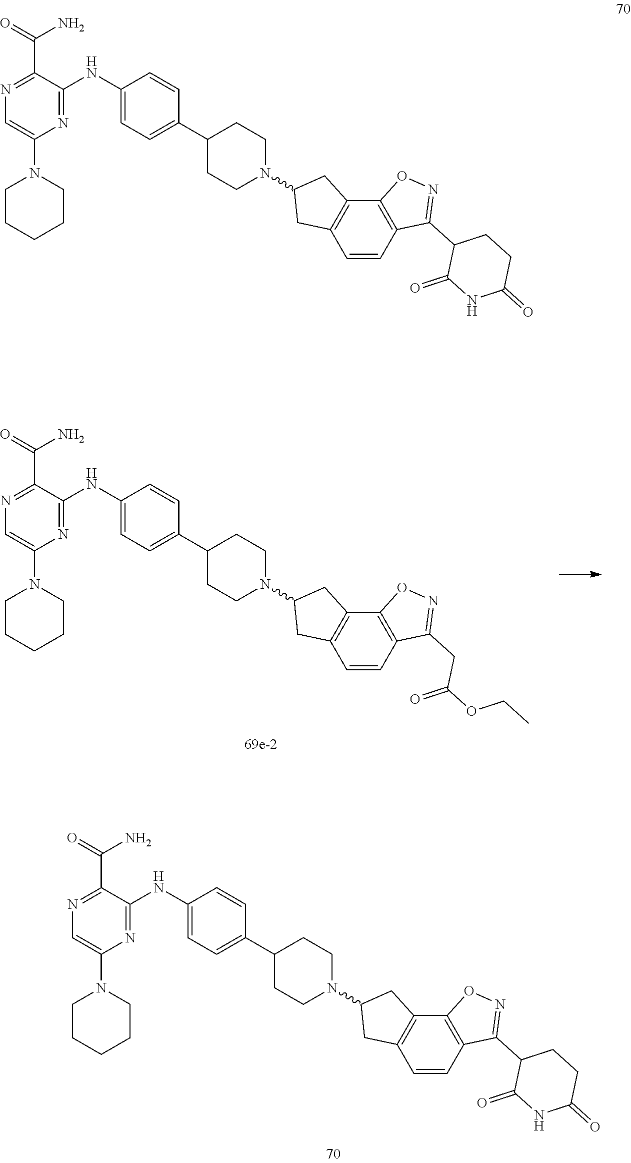 [SMILES: NC(=O)c1ncc(N2CCCCC2)nc1Nc1ccc(C2CCN(C3Cc4ccc5c(C6CCC(=O)NC6=O)noc5c4C3)CC2)cc1 ; CCOC(=O)Cc1noc2c3c(ccc12)CC(N1CCC(c2ccc(Nc4nc(N5CCCCC5)cnc4C(N)=O)cc2)CC1)C3 ; NC(=O)c1ncc(N2CCCCC2)nc1Nc1ccc(C2CCN(C3Cc4ccc5c(C6CCC(=O)NC6=O)noc5c4C3)CC2)cc1]

Intermediate 69e-2 (56 mg), acrylamide (6.38 mg, 0.090 mmol), and tetrahydrofuran (5 mL) were added to a reaction flask in sequence, and the mixture was cooled to 0° C. under N₂ atmosphere. Potassium tert-butoxide (1 mol/L, 0.063 mL, 0.063 mmol) was added, and the mixture was reacted at 0° C. After the reaction was completed, as confirmed by TLC, the reaction solution was added dropwise to an icy saturated aqueous ammonium chloride solution, and 50 mL of ethyl acetate was added for extraction. The organic phase was separated, and the aqueous phase was extracted twice with 20 mL of ethyl acetate. The organic phases were combined, washed with saturated brine, dried over anhydrous sodium sulfate, and filtered, and the filtrate was purified by silica gel column chromatography (dichloromethane:MeOH=0-4%) to give compound 70 (25 mg).

MS(ESI, [M+H]+) m/z: 649.56.

¹H NMR (500 MHz, DMSO-d6) δ 11.28 (s, 1H), 11.09 (s, 1H), 7.73 (d, J=2.9 Hz, 1H), 7.66 (s, 1H), 7.63 (d, J=8.0 Hz, 1H), 7.52 (d, J=8.1 Hz, 2H), 7.31 (d, J=2.9 Hz, 1H), 7.27 (d, J=8.1 Hz, 1H), 7.19 (d, J=8.2 Hz, 2H), 4.57 (dd, J=11.8, 5.0 Hz, 1H), 3.67 (t, J=5.3 Hz, 4H), 3.43 (p, J=7.6 Hz, 1H), 3.29 (d, J=7.7 Hz, 1H), 3.21 (dd, J=16.3, 7.6 Hz, 1H), 3.05-2.97 (m, 4H), 2.77 (ddd, J=17.2, 11.9, 5.3 Hz, 1H), 2.61 (dt, J=17.3, 4.3 Hz, 1H), 2.48-2.44 (m, 1H), 2.22-2.10 (m, 3H), 1.81-1.74 (m, 2H), 1.72-1.53 (in, 9H).

### Example 71: Synthesis of Compound 71

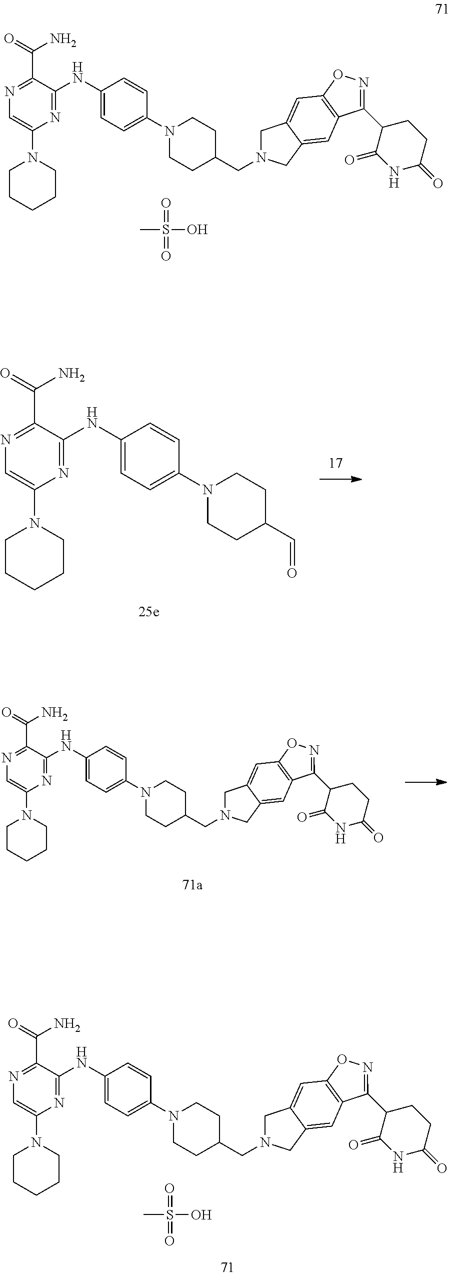

### Step 1: Preparation of Intermediate 71a

Intermediate 25e (138 mg), 17 (80 mg), sodium acetate (24 mg), and dichloroethane/isopropanol (5:1, 25 mL) were added to a reaction flask in sequence. After the mixture was stirred at room temperature for 30 min, sodium cyanoborohydride (21 mg) was added, and the mixture was reacted at room temperature. After the reaction was completed, as confirmed by TLC, 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 71a (72 mg).

MS(ESI, [M+H]⁺) m/z: 664.5.

### Step 2: Preparation of Compound 71

Intermediate 71a (72 mg), methanesulfonic acid (11 mg), and methanol (20 mL) were added to a reaction flask in sequence, and the mixture was reacted at room temperature. After the reaction was completed, as confirmed by TLC, the reaction solution was concentrated to give compound 71 (83 mg).

MS(ESI, [M+H]⁺) m/z: 664.5.

1H NMR (500 MHz, DMSO-d6) δ 11.14 (s, 1H), 10.49 (s, 1H), 7.89 (d, J=5.0 Hz, 1H), 7.86 (s, 1H), 7.69 (d, J=39.8 Hz, 2H), 7.52 (s, 2H), 7.36-7.24 (m, 1H), 7.00 (s, 1H), 5.12-4.88 (m, 2H), 4.80-4.57 (m, 3H), 3.75-3.61 (m, 6H), 3.17 (s, 1H), 2.87-2.59 (m, 3H), 2.31 (s, 3H), 2.27-2.17 (m, 1H), 1.98 (s, 3H), 1.73-1.53 (m, 6H), 1.47 (s, 1H).

### Example 72: Synthesis of Compound 72

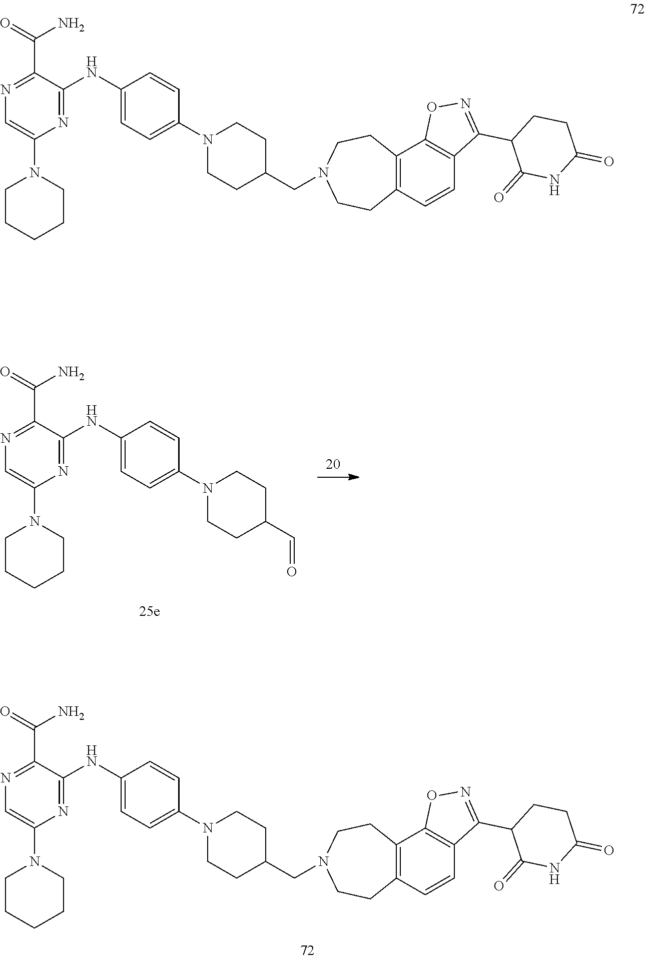 [SMILES: NC(=O)c1ncc(N2CCCCC2)nc1Nc1ccc(N2CCC(CN3CCc4ccc5c(C6CCC(=O)NC6=O)noc5c4CC3)CC2)cc1 ; NC(=O)c1ncc(N2CCCCC2)nc1Nc1ccc(N2CCC(C=O)CC2)cc1 ; NC(=O)c1ncc(N2CCCCC2)nc1Nc1ccc(N2CCC(CN3CCc4ccc5c(C6CCC(=O)NC6=O)noc5c4CC3)CC2)cc1]

### Step 1: Synthesis of Compound 72

Intermediate 25e (102 mg), 20 (84 mg), dichloroethane/isopropanol (5:1, 25 mL), and sodium acetate (20 mg) were added to a reaction flask in sequence. After the mixture was stirred at room temperature for 30 min, sodium cyanoborohydride (31 mg) was added, and the mixture was reacted at room temperature. After the reaction was completed, as confirmed by TLC, 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 72 (65 mg).

MS(ESI, [M+H]⁺) m/z: 692.4.

¹H NMR (500 MHz, DMSO-d6) δ 11.07 (d, J=10.7 Hz, 2H), 7.69 (d, J=2.9 Hz, 1H), 7.60 (s, 1H), 7.54 (d, J=7.9 Hz, 1H), 7.47-7.39 (m, 2H), 7.26 (d, J=2.8 Hz, 1H), 7.18 (d, J=8.1 Hz, 1H), 6.97-6.87 (m, 2H), 4.55 (dd, J=11.9, 5.0 Hz, 1H), 3.69-3.58 (m, 6H), 3.20-3.12 (m, 2H), 3.10-2.98 (m, 2H), 2.77 (ddd, J=17.2, 12.0, 5.3 Hz, 1H), 2.69-2.58 (m, 6H), 2.47 (dd, J=12.2, 4.2 Hz, 1H), 2.35 (d, J=7.1 Hz, 2H), 2.18 (dq, J=13.7, 5.0 Hz, 1H), 1.88-1.80 (m, 2H), 1.65 (q, J=6.2 Hz, 3H), 1.58 (q, J=5.6, 5.2 Hz, 4H), 1.31-1.25 (m, 2H).

### Examples 73 and 74: Synthesis of Compounds 73 and 74

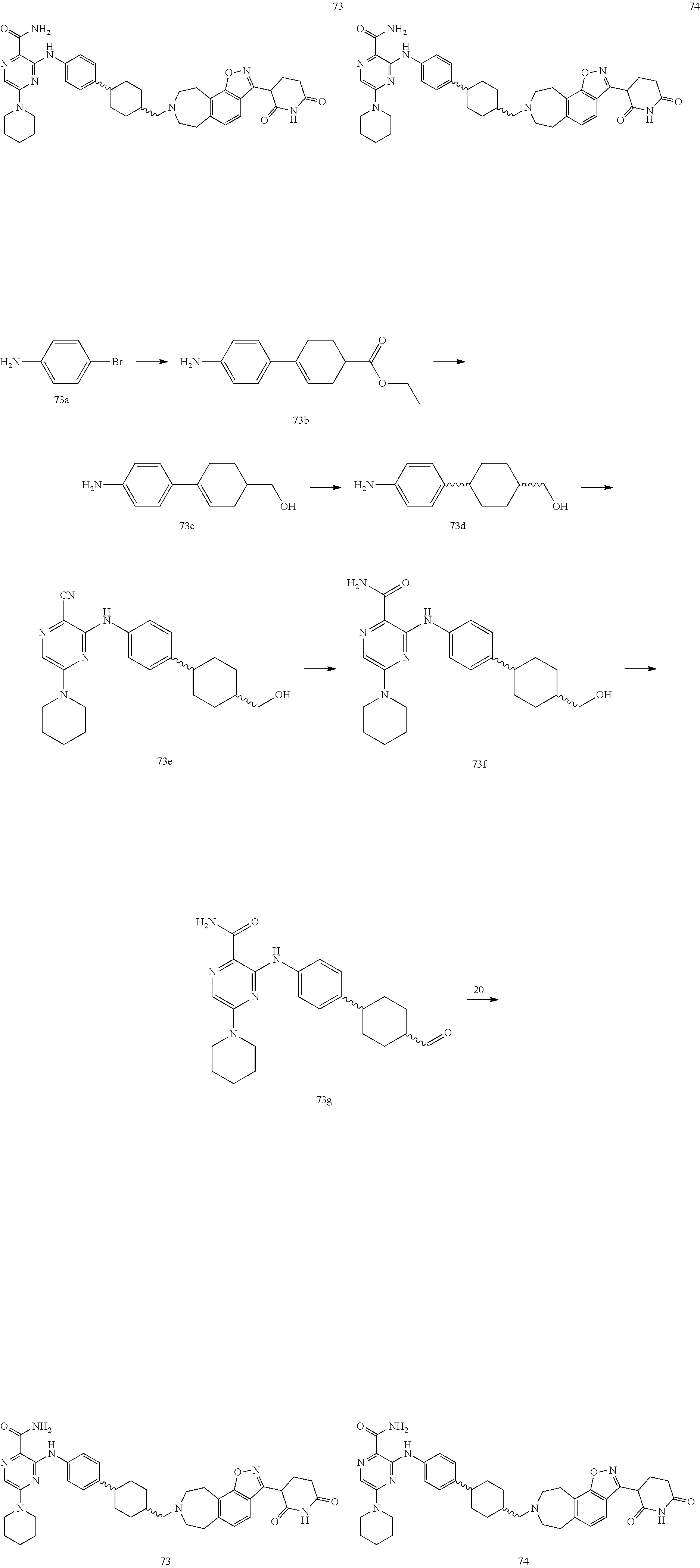

### Step 1: Preparation of Intermediate 73b

Intermediate 73a (2.0 g), ethyl 4-(4,4,5,5-tetramethyl-1,3,2-dioxolan-2-yl)benzoate (3.26 g), potassium carbonate (3.21 g), PdCl₂(dppf) (0.85 g), water (5 mL), and 1,4-dioxane (25 mL) were added to a reaction flask in sequence, and the mixture was purged three times with nitrogen, heated to 100° C., and reacted. After the reaction was completed, as confirmed by TLC, the reaction solution was cooled to room temperature, and 80 L of ethyl acetate and 140 mL of water were added to the residue. The organic phase was separated, washed with a saturated sodium chloride solution, dried over anhydrous sodium sulfate, filtered, and concentrated. The concentrate was separated and purified by silica gel column chromatography to give intermediate 73b (2.0 g).

MS(ESI, [M+H]⁺) m/z: 246.1.

1H NMR (500 MHz, DMSO-d6) δ 7.11-7.05 (m, 2H), 6.54-6.47 (m, 2H), 5.89 (td, J=3.3, 1.5 Hz, 1H), 4.08 (qd, J=7.1, 2.1 Hz, 2H), 2.58-2.52 (m, 1H), 2.45-2.23 (m, 4H), 2.08-2.01 (m, 1H), 1.66 (dddd, J=12.8, 11.0, 9.9, 5.8 Hz, 1H), 1.19 (t, J=7.1 Hz, 3H), 1.07 (s, 2H).

### Step 2: Preparation of Intermediate 73c

A solution of lithium aluminum hydride in tetrahydrofuran (9.8 mL, 1 M) was added dropwise to a stirred solution of 73b in tetrahydrofuran (20 mL) at 0° C. After the dropwise addition was completed, the mixture was reacted at 0° C. After the reaction was completed, as confirmed by TLC, the reaction solution was slowly added dropwise to 100 mL of ice water, and 60 mL of ethyl acetate was added. The organic phase was separated, washed with a saturated sodium chloride solution, dried over anhydrous sodium sulfate, filtered, and concentrated to give intermediate 73c (0.68 g).

MS(ESI, [M+H]⁺) m/z: 204.4.

### Step 3: Preparation of Intermediate 73d

Intermediate 73c (0.6 g), 10% palladium on carbon (0.3 g), glacial acetic acid (0.1 g), and methanol (30 mL) were added to a reaction flask in sequence, and the mixture was purged three times with hydrogen and reacted overnight. The palladium on carbon was removed by filtration under vacuum, and the residue was concentrated to give intermediate 73d (0.62 g).

MS(ESI, [M+H]⁺) m/z: 206.1.

### Step 4: Preparation of Intermediate 73e

25b (0.4 g), 73d (0.62 g), BINAP (0.11 g), cesium carbonate (1.75 g), palladium acetate (0.04 g), and 1,4-dioxane (30 mL) were added to a reaction flask in sequence, and the mixture was purged three times with nitrogen, heated to 100° C., and reacted. After the reaction was completed, as confirmed by TLC, the reaction solution was cooled to room temperature, and 80 L of ethyl acetate and 140 mL of water were added to the residue. The organic phase was separated, washed with a saturated sodium chloride solution, dried over anhydrous sodium sulfate, filtered, and concentrated. The concentrate was separated and purified by silica gel column chromatography to give intermediate 73e (0.63 g).

MS(ESI, [M+H]⁺) m/z: 392.3.

1H NMR (500 MHz, DMSO-d6) δ 8.87 (s, 1H), 7.82 (s, 1H), 7.47-7.41 (m, 2H), 7.18-7.09 (m, 2H), 4.43-4.29 (m, 1H), 3.62 (t, J=5.5 Hz, 4H), 3.45 (dd, J=7.3, 5.4 Hz, 1H), 3.25 (t, J=5.8 Hz, 1H), 1.86-1.77 (m, 2H), 1.76-1.66 (m, 2H), 1.63 (q, J=6.5 Hz, 3H), 1.58-1.49 (m, 6H), 1.46-1.35 (m, 1H), 1.02 (qd, J=13.1, 3.7 Hz, 1H), 0.90-0.79 (m, 1H).

### Step 5: Preparation of Intermediate 73f

Intermediate 73e (0.63 g), cesium carbonate (0.52 g), water (5 mL), DMSO (20 mL), and methanol (5 mL) were added to a reaction flask in sequence. The reaction solution was cooled to 0° C., and 30 wt % hydrogen peroxide (0.54 g) was added dropwise. After the dropwise addition was completed, the mixture was allowed to react at room temperature. After the reaction was completed, as confirmed by TLC, 50 mL of water was added to the reaction solution, and then a saturated aqueous sodium sulfite solution was added to neutralize 30 wt % hydrogen peroxide. The mixture was stirred for 10 min and filtered, and the filter cake was collected and dried to give intermediate 73f (0.55 g).

MS(ESI, [M+H]⁺) m/z: 410.4.

### Step 6: Preparation of Intermediate 73g

Intermediate 73f (0.55 g), dichloromethane (25 mL), and N,N-diisopropylethylamine (0.46 g) were added to a reaction flask in sequence, and the mixture was cooled to 0° C. Sulfur trioxide pyridine (0.87 g) was weighed out and dissolved in DMSO (2 mL), and the solution was added dropwise to the reaction solution. After the dropwise addition was completed, the mixture was allowed to react at room temperature. After the reaction was completed, as confirmed by TLC, ethyl acetate (80 mL) and water (120 mL) were added to the reaction solution to separate the organic phase of the filtrate. The organic phase was separated, washed with a saturated sodium chloride solution, dried over anhydrous sodium sulfate, filtered, and concentrated. The concentrate was separated and purified by silica gel column chromatography to give intermediate 73g (0.38 g).

MS(ESI, [M+H]⁺) m/z: 408.3.

### Step 7: Preparation of Compounds 73 and 74

Intermediate 73g (250 mg), 20 (206 mg), dichloroethane/isopropanol (5:1, 25 mL), and sodium acetate (50 mg) were added to a reaction flask in sequence. After the mixture was stirred at room temperature for 30 min, sodium cyanoborohydride (77 mg) was added, and the mixture was reacted at room temperature. After the reaction was completed, as confirmed by TLC, 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give a mixture of compounds 73 and 74, which was subjected to preparative resolution to give compound 73 (15 mg) and compound 74 (20 mg) in sequence.

The conditions of preparative chromatography were as follows: instrument and preparative column: YMC high pressure preparative chromatograph was used, and the preparative column was Ultimate Polar RP. Mobile phase system: acetonitrile/10 nM aqueous ammonium acetate solution; isocratic elution: acetonitrile/10 nM aqueous ammonium acetate solution=45/55.

### Compound 73

MS(ESI, [M+H]⁺) m/z: 691.5.

1H NMR (500 MHz, DMSO-d6) δ 11.27 (s, 1H), 11.08 (s, 1H), 7.69 (d, J=36.5 Hz, 2H), 7.53 (dd, J=13.3, 8.0 Hz, 3H), 7.31 (s, 1H), 7.18 (d, J=8.0 Hz, 3H), 4.55 (dd, J=11.9, 5.0 Hz, 1H), 3.68 (t, J=5.3 Hz, 4H), 3.10 (dt, J=56.7, 4.7 Hz, 4H), 2.77 (ddd, J=17.1, 12.2, 5.4 Hz, 1H), 2.63 (dd, J=18.8, 7.7 Hz, 5H), 2.43 (d, J=12.4 Hz, 2H), 2.30 (d, J=6.8 Hz, 2H), 2.22-2.12 (m, 1H), 1.88 (dd, J=51.3, 12.7 Hz, 4H), 1.72-1.63 (m, 2H), 1.65-1.52 (m, 5H), 1.46 (q, J=12.6 Hz, 2H), 1.03 (q, J=12.6 Hz, 2H).

### Compound 74

MS(ESI, [M+H]⁺) m/z: 691.5.

1H NMR (500 MHz, DMSO-d6) δ 11.29 (s, 1H), 11.09 (s, 1H), 7.73 (d, J=2.9 Hz, 1H), 7.66 (s, 1H), 7.53 (t, J=8.5 Hz, 3H), 7.31 (d, J=2.9 Hz, 1H), 7.20 (dd, J=17.3, 8.1 Hz, 3H), 4.55 (dd, J=11.9, 5.0 Hz, 1H), 3.68 (t, J=5.3 Hz, 4H), 3.11 (dt, J=56.6, 5.0 Hz, 4H), 2.77 (ddd, J=17.1, 12.0, 5.3 Hz, 1H), 2.72-2.56 (m, 5H), 2.52 (s, 2H), 2.47 (dd, J=12.2, 4.3 Hz, 2H), 2.18 (dt, J=13.0, 4.9 Hz, 1H), 1.96 (s, 1H), 1.72 (d, J=7.4 Hz, 2H), 1.66 (dt, J=11.1, 5.6 Hz, 4H), 1.63-1.54 (m, 8H).

### Example 75: Synthesis of Compound 75

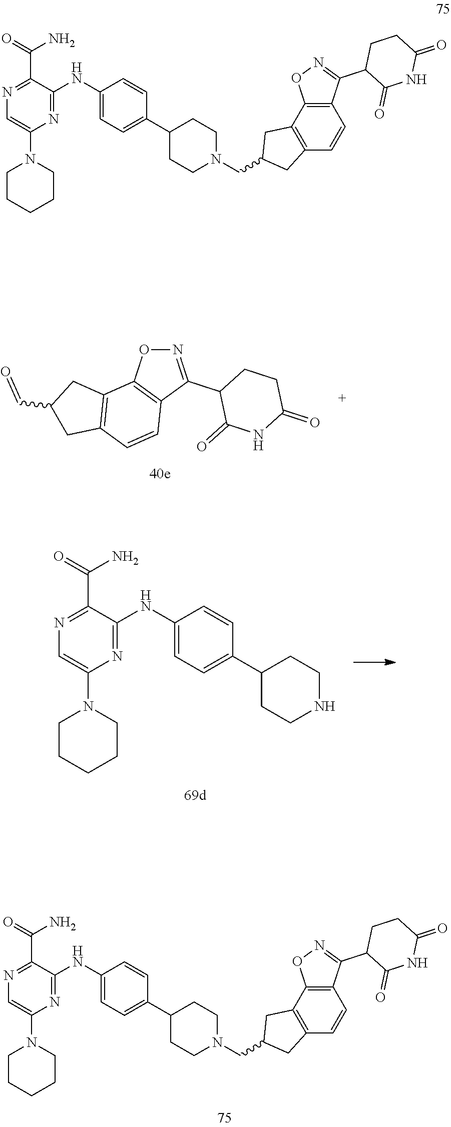

Intermediate 40e (80 mg), 1,2-dichloroethane (6 mL), isopropanol (2 mL), intermediate 69d (101 mg), acetic acid (8.00 mg, 0.133 mmol), and sodium cyanoborohydride (33.5 mg, 0.533 mmol) were added to a reaction flask in sequence, and the mixture was stirred and reacted at room temperature. After the reaction was completed, 2 mL of a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography (dichloromethane:MeOH=0-5%) to give compound 75 (40 mg).

MS(ESI, [M+H]+) m/z: 663.4.

¹H NMR (500 MHz, DMSO-d6) δ 11.29 (s, 1H), 11.09 (s, 1H), 7.74 (d, J=2.9 Hz, 1H), 7.66 (s, 1H), 7.62 (d, J=8.0 Hz, 1H), 7.53 (d, J=8.1 Hz, 2H), 7.32 (d, J=2.9 Hz, 1H), 7.27 (d, J=8.1 Hz, 1H), 7.20 (d, J=8.1 Hz, 2H), 4.57 (dd, J=11.8, 5.0 Hz, 1H), 3.68 (t, J=5.2 Hz, 4H), 3.20 (td, J=17.8, 15.2, 7.4 Hz, 2H), 3.02 (s, 2H), 2.97-2.68 (m, 5H), 2.60 (dd, J=17.6, 4.4 Hz, 1H), 2.46 (d, J=4.3 Hz, 1H), 2.37 (s, 2H), 2.19 (dq, J=13.5, 4.5 Hz, 1H), 2.13-1.94 (m, 2H), 1.85-1.71 (m, 3H), 1.66 (q, J=6.0 Hz, 3H), 1.60 (q, J=5.5, 5.1 Hz, 4H).

### Example 76: Synthesis of Compound 76

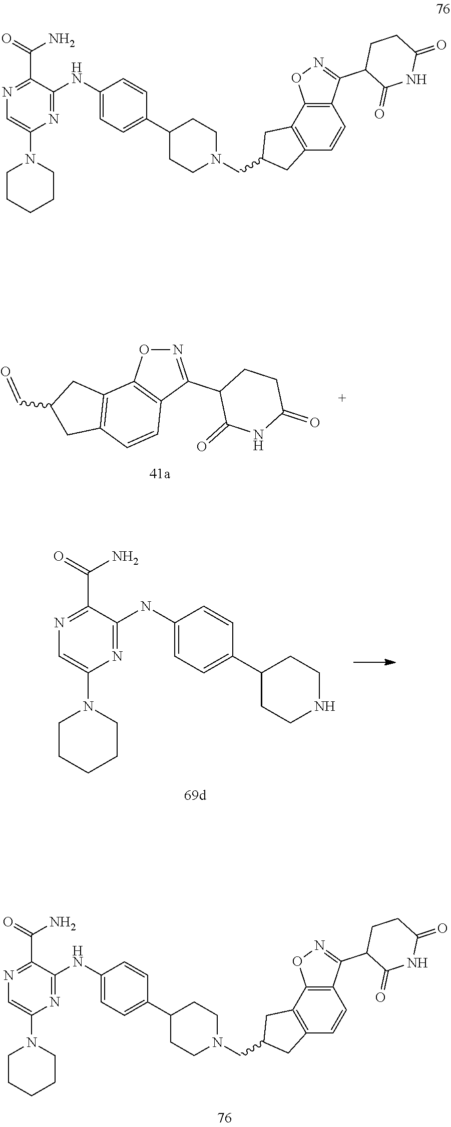

Intermediate 41a (80 mg), 1,2-dichloroethane (6 mL), isopropanol (2 mL), intermediate 69d (101 mg), acetic acid (8.00 mg, 0.133 mmol), and sodium cyanoborohydride (33.5 mg, 0.533 mmol) were added to a reaction flask in sequence, and the mixture was stirred and reacted at room temperature. After the reaction was completed, 2 mL of a saturated sodium bicarbonate solution was added to the reaction solution to neutralize acetic acid, and then 50 mL of dichloromethane and 100 mL of water were added for extraction. The organic phase was separated, and the aqueous phase was extracted twice with 20 mL of dichloromethane. The organic phases were combined, washed with saturated brine, dried over anhydrous sodium sulfate, and filtered, and the filtrate was purified by silica gel column chromatography (dichloromethane:MeOH=0-5%) to give compound 76 (62 mg).

MS(ESI, [M+H]+) m/z: 663.4.

¹H NMR (500 MHz, DMSO-d6) δ 11.30 (s, 1H), 11.09 (s, 1H), 7.74 (d, J=2.9 Hz, 1H), 7.66 (s, 1H), 7.62 (d, J=8.0 Hz, 1H), 7.54 (d, J=8.1 Hz, 2H), 7.32 (d, J=2.9 Hz, 1H), 7.28 (d, J=8.1 Hz, 1H), 7.21 (d, J=8.1 Hz, 2H), 4.57 (ddd, J=11.9, 5.0, 1.5 Hz, 1H), 3.68 (t, J=5.4 Hz, 4H), 3.21 (dd, J=26.7, 12.2 Hz, 2H), 3.05 (s, 2H), 2.98-2.74 (m, 5H), 2.64-2.59 (m, 1H), 2.49-2.45 (m, 1H), 2.19 (dp, J=13.3, 4.4 Hz, 3H), 2.14-1.88 (m, 2H), 1.78 (s, 3H), 1.66 (q, J=5.9, 5.5 Hz, 3H), 1.59 (dq, J=8.4, 5.4, 4.9 Hz, 4H).

### Example 77: Synthesis of Compound 77

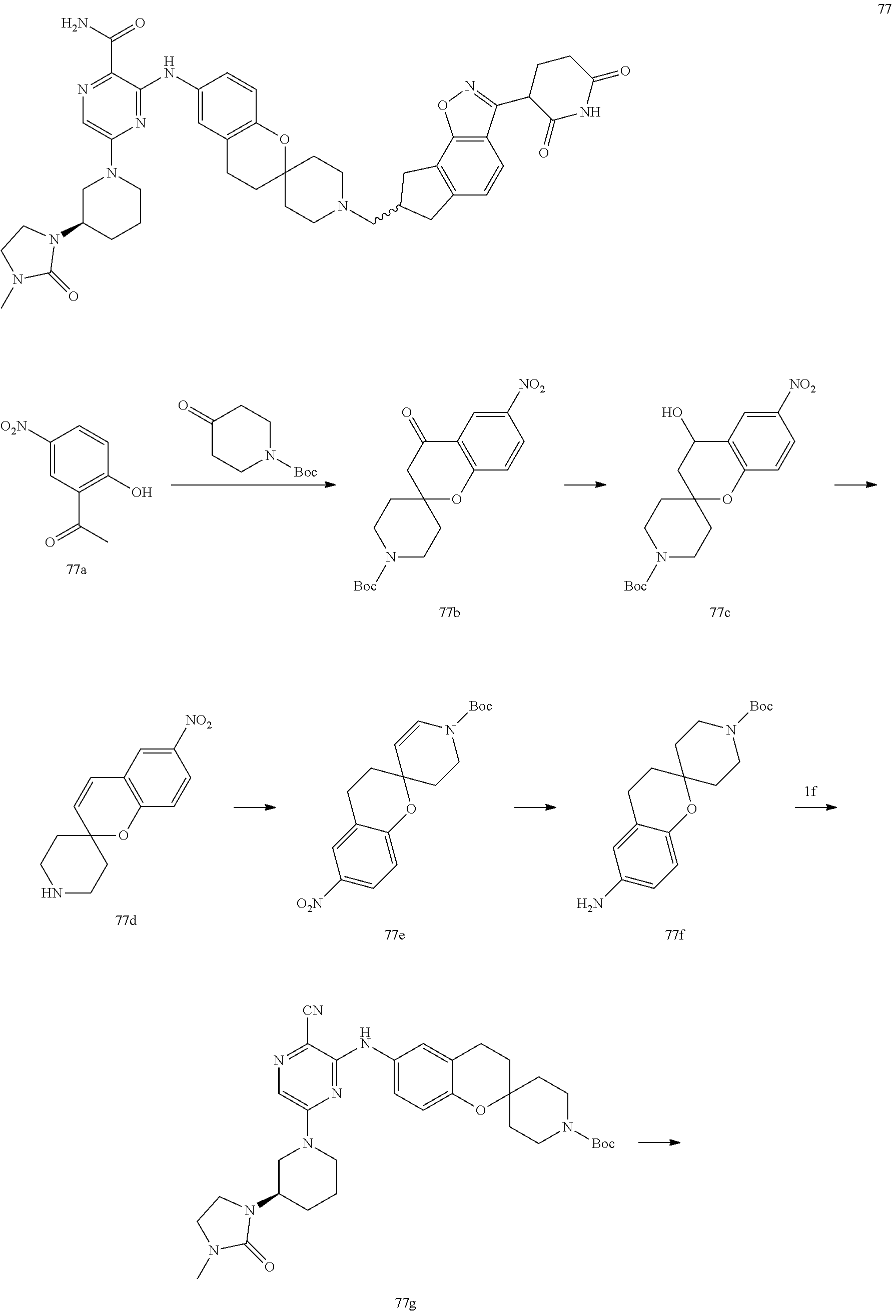

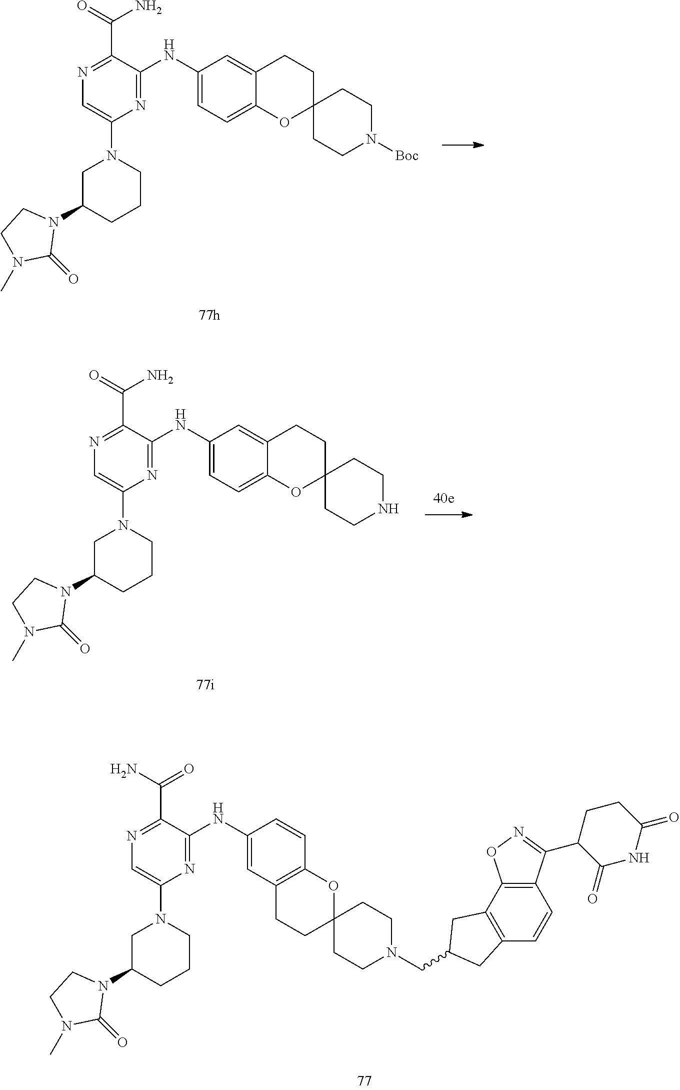 [SMILES: CN1CCN([C@@H]2CCCN(c3cnc(C(N)=O)c(Nc4ccc5c(c4)CCC4(CCN(C(=O)OC(C)(C)C)CC4)O5)n3)C2)C1=O ; CN1CCN([C@@H]2CCCN(c3cnc(C(N)=O)c(Nc4ccc5c(c4)CCC4(CCNCC4)O5)n3)C2)C1=O ; CN1CCN([C@@H]2CCCN(c3cnc(C(N)=O)c(Nc4ccc5c(c4)CCC4(CCN(CC6Cc7ccc8c(C9CCC(=O)NC9=O)noc8c7C6)CC4)O5)n3)C2)C1=O]

### Step 1: Preparation of Intermediate 77b

77a (10 g), tert-butyl 4-oxopiperidine-1-carboxylate (11.00 g), pyrrolidine (1.178 g), and toluene (180 mL) were added to a reaction flask in sequence, and the mixture was reacted at 120° C. After the reaction was completed, a mixed solvent of 500 mL of ethyl acetate and 300 mL of water was poured into the reaction solution. The organic phase was separated, dried over anhydrous sodium sulfate, filtered, and concentrated by evaporation at reduced pressure to remove the solvent, and the residue was purified by silica gel column chromatography to give 77b (16.57 g).

¹H NMR (500 MHz, DMSO) δ 8.47 (d, J=2.9 Hz, 1H), 8.42 (dd, J=9.0, 2.9 Hz, 1H), 7.34 (d, J=9.1 Hz, 1H), 3.74 (d, J=13.4 Hz, 2H), 3.13 (s, 2H), 3.01 (s, 2H), 1.91 (dq, J=14.8, 3.0 Hz, 2H), 1.70 (ddd, J=13.9, 11.6, 4.7 Hz, 2H), 1.40 (s, 9H).

### Step 2: Preparation of Intermediate 77c

77b (16 g), THF (80 mL), and sodium borohydride (3.34 g, 88 mmol) were added to a reaction flask in sequence under nitrogen atmosphere, and the mixture was reacted at room temperature. After the reaction was completed, the reaction solution was poured into a mixed solution of water (200 mL) and ethyl acetate (300 mL) containing crushed ice. The organic phase was separated, dried over anhydrous sodium sulfate, filtered, and concentrated to give 77c (19.28 g).

¹H NMR (500 MHz, DMSO) δ 8.34 (dd, J=3.0, 0.9 Hz, 1H), 8.05 (dd, J=9.1, 2.9 Hz, 1H), 7.01 (d, J=9.0 Hz, 1H), 5.83 (d, J=6.0 Hz, 1H), 4.79 (dt, J=9.5, 5.9 Hz, 1H), 3.70 (dd, J=25.1, 13.6 Hz, 2H), 3.33 (s, 1H), 3.08 (s, 1H), 2.22 (dd, J=13.6, 6.1 Hz, 1H), 1.84-1.68 (m, 4H), 1.61 (ddd, J=13.7, 11.1, 4.6 Hz, 1H), 1.41 (s, 9H).

### Step 3: Preparation of Intermediate 77d

77c (5 g), trifluoroacetic acid (40 mL), and triethylsilane (18.46 g, 25.4 mL, 159 mmol) were added to a reaction flask in sequence, and the mixture was heated to 70° C. and reacted. After the reaction was completed, the reaction solution was concentrated by evaporation at reduced pressure to remove the solvent, 200 mL of dichloromethane and 200 mL of water were added to the residue, and the pH was adjusted to 11-12 with sodium hydroxide. The organic phase was separated, dried over anhydrous sodium sulfate, filtered, and concentrated by evaporation at reduced pressure to remove the solvent, and the residue was purified by silica gel column chromatography to give 77d (3.18 g).

MS(ESI, [M+H]⁺) m/z: 247.1.

### Step 4: Preparation of Intermediate 77e

77d (3 g), dichloromethane (100 mL), triethylamine (5.71 mL), and Boc anhydride (3.29 g) were added to a reaction flask, and the mixture was reacted at room temperature. After the reaction was completed, the reaction solution was mixed with silica gel and purified by column chromatography to give 77e (4.25 g).

MS(ESI, [M+H]+) m/z: 347.2.

### Step 5: Preparation of Intermediate 77f

77e (4.25 g), 10% palladium on carbon (500 mg), and methanol (20 mL) were added to a reaction flask in sequence, and the mixture was purged with hydrogen and reacted overnight at room temperature. After the reaction was completed, the reaction solution was filtered under vacuum, and the filtrate was concentrated to give 77f (3.02 g).

MS(ESI, [M+H]⁺) m/z: 319.3.

### Step 6: Preparation of Intermediate 77g

Intermediate 1f (1 g), 77f (1.191 g), cesium carbonate (3.05 g), BINAP (0.194 g), palladium acetate (0.070 g), and 1,4-dioxane (20 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 77g (1.6 g).

¹H NMR (500 MHz, DMSO-d6) δ 8.80 (s, 1H), 7.77 (s, 1H), 7.30 (d, J=2.6 Hz, 1H), 7.16 (dd, J=8.7, 2.6 Hz, 1H), 6.70 (d, J=8.8 Hz, 1H), 4.36-4.16 (m, 2H), 3.70 (d, J=12.8 Hz, 2H), 3.60-3.50 (m, 1H), 3.25 (dddd, J=23.3, 21.0, 11.5, 6.5 Hz, 4H), 3.17-2.79 (m, 4H), 2.67 (s, 5H), 1.82-1.63 (m, 7H), 1.57-1.46 (m, 3H), 1.41 (s, 9H).

### Step 7: Preparation of Intermediate 77h

Intermediate 77g (1.6 g), DMSO (20 mL), and cesium carbonate (1.03 g) were added to a single-necked flask in sequence. 30 wt % hydrogen peroxide (0.9 mL) was added under an ice bath. After the mixture was stirred for 5 min, the ice bath was removed, and the mixture was reacted at room temperature. After the reaction was completed, 300 mL of a saturated sodium sulfite solution was added to the residue. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 77h (1.73 g).

MS(ESI, [M+H]+) m/z: 621.55.

### Step 8: Preparation of Intermediate 771

77h (1.7 g), dichloromethane (20 mL), and TFA (5 mL) were added to a single-necked flask in sequence, and the mixture was reacted at room temperature. After the reaction was completed, the system was added to 200 mL of a saturated sodium bicarbonate solution, and then 200 mL of dichloromethane was added for extraction. The organic phase was dried over anhydrous sodium sulfate, filtered, and concentrated by evaporation at reduced pressure to remove the solvent, thus giving 771 (1.21 g).

MS(ESI, [M+H]+) m/z: 521.49.

### Step 9: Preparation of Compound 77

40e (60 mg), isopropanol (1 mL), 1,2-dichloroethane (5.00 mL), and 771 (104 mg) were added to a reaction flask in sequence, and sodium cyanoborohydride (37 mg) was added. The mixture was reacted at room temperature. After the reaction was completed, the reaction solution was mixed with silica gel and purified by column chromatography to give 77 (23 mg).

MS(ESI, [M+H]+) m/z: 803.64.

¹H NMR (500 MHz, DMSO-d₆) δ 11.08 (s, 1H), 11.04 (s, 1H), 7.73 (s, 1H), 7.61 (d, J=10.4 Hz, 2H), 7.48 (s, 1H), 7.37-7.22 (m, 2H), 7.10 (d, J=8.5 Hz, 1H), 6.69 (d, J=8.6 Hz, 1H), 4.56 (dd, J=11.5, 5.0 Hz, 1H), 4.31 (dd, J=56.0, 12.6 Hz, 2H), 3.61 (dt, J=11.3, 6.6 Hz, 1H), 3.35 (s, 2H), 3.30-3.13 (m, 4H), 2.93 (ddd, J=53.2, 41.3, 14.0 Hz, 5H), 2.79-2.55 (m, 9H), 2.45 (d, J=4.0 Hz, 1H), 2.37 (s, 4H), 2.18 (d, J=13.8 Hz, 1H), 1.90-1.53 (m, 10H).

### Example 78: Synthesis of Compound 78

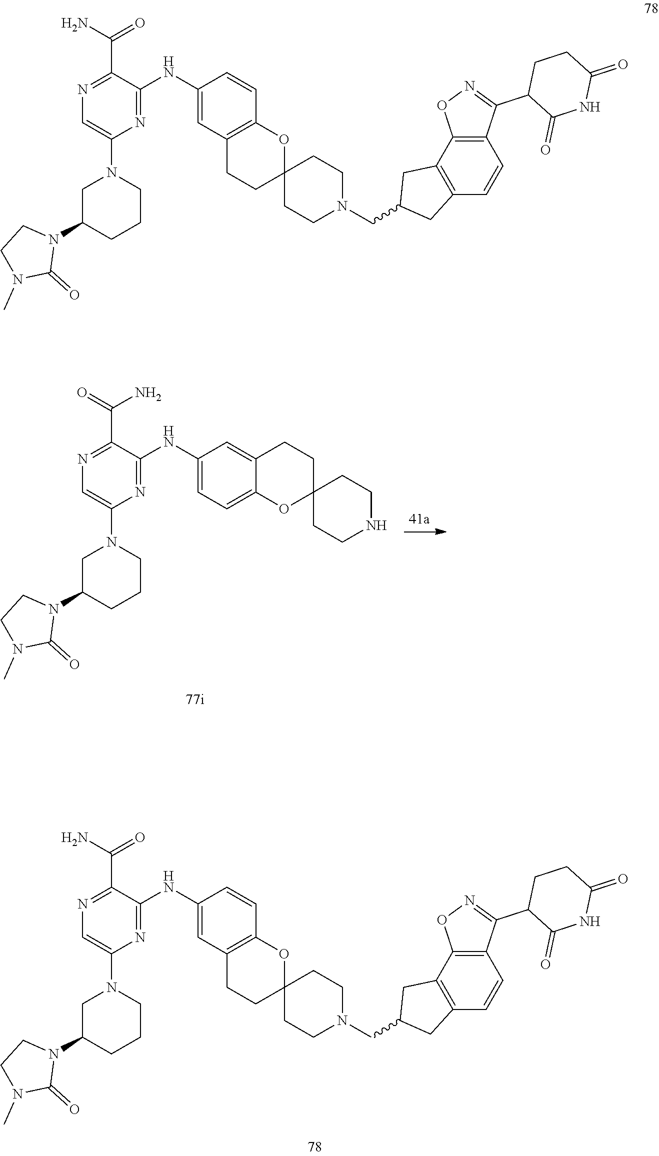 [SMILES: CN1CCN([C@@H]2CCCN(c3cnc(C(N)=O)c(Nc4ccc5c(c4)CCC4(CCN(CC6Cc7ccc8c(C9CCC(=O)NC9=O)noc8c7C6)CC4)O5)n3)C2)C1=O ; CN1CCN([C@@H]2CCCN(c3cnc(C(N)=O)c(Nc4ccc5c(c4)CCC4(CCNCC4)O5)n3)C2)C1=O ; CN1CCN([C@@H]2CCCN(c3cnc(C(N)=O)c(Nc4ccc5c(c4)CCC4(CCN(CC6Cc7ccc8c(C9CCC(=O)NC9=O)noc8c7C6)CC4)O5)n3)C2)C1=O]

### Step 1: Preparation of Compound 78

41a (60 mg), isopropanol (1 mL), 1,2-dichloroethane (5.00 mL), and 771 (104 mg) were added to a reaction flask in sequence, and sodium cyanoborohydride (37 mg) was added. The mixture was reacted at room temperature. After the reaction was completed, the reaction solution was mixed with silica gel and purified by column chromatography to give 78 (10 mg).

MS(ESI, [M+H]+) m/z: 803.69.

¹H NMR (500 MHz, DMSO-d₆) δ 11.08 (s, 1H), 11.04 (s, 1H), 7.73 (s, 1H), 7.61 (d, J=10.4 Hz, 2H), 7.48 (s, 1H), 7.37-7.22 (m, 2H), 7.10 (d, J=8.5 Hz, 1H), 6.69 (d, J=8.6 Hz, 1H), 4.56 (dd, J=11.5, 5.0 Hz, 1H), 4.31 (dd, J=56.0, 12.6 Hz, 2H), 3.61 (dt, J=11.3, 6.6 Hz, 1H), 3.35 (s, 2H), 3.30-3.13 (m, 4H), 2.93 (ddd, J=53.2, 41.3, 14.0 Hz, 5H), 2.79-2.55 (m, 9H), 2.45 (d, J=4.0 Hz, 1H), 2.37 (s, 4H), 2.18 (d, J=13.8 Hz, 1H), 1.90-1.53 (m, 10H).

### Example 79: Synthesis of Compound 79

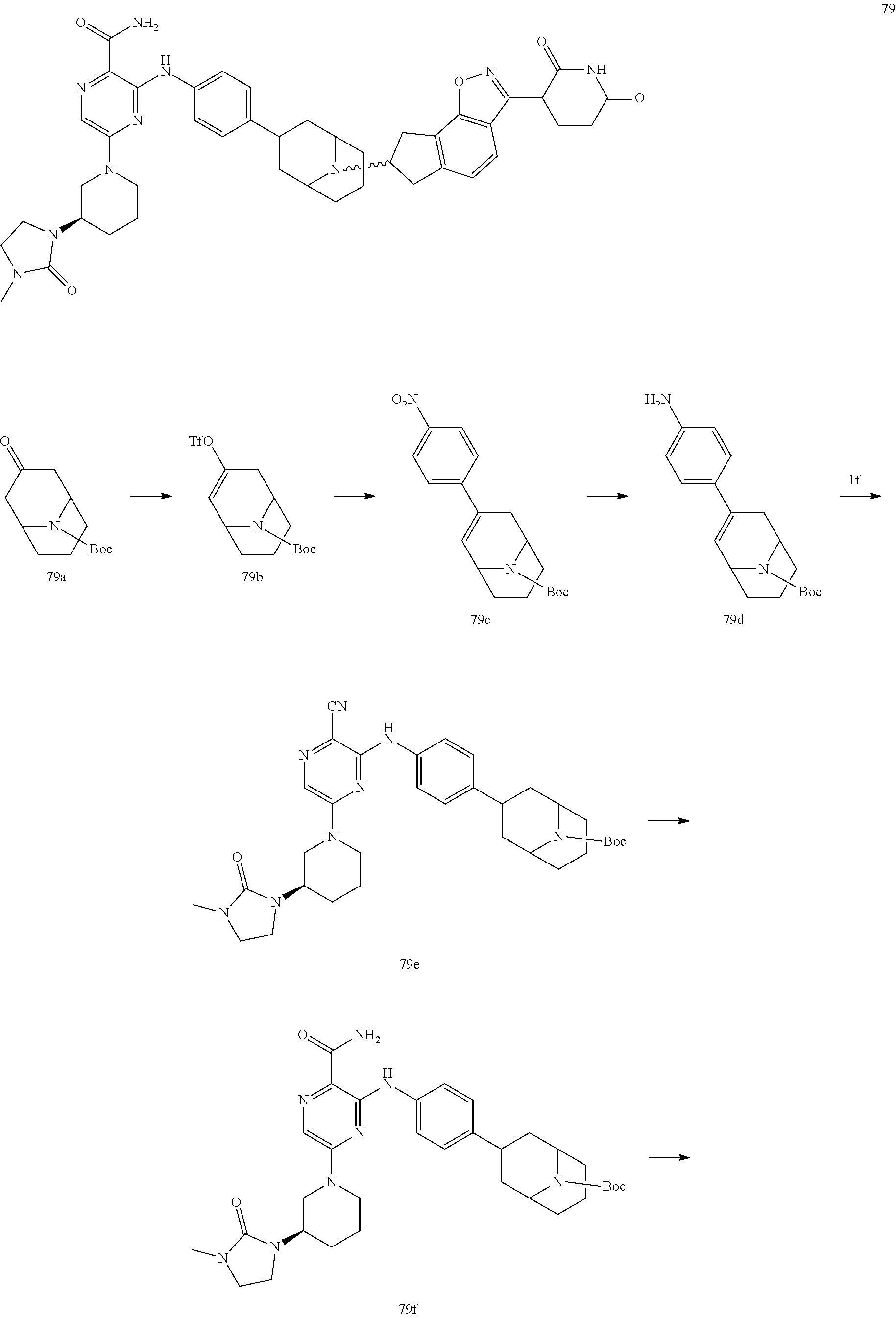

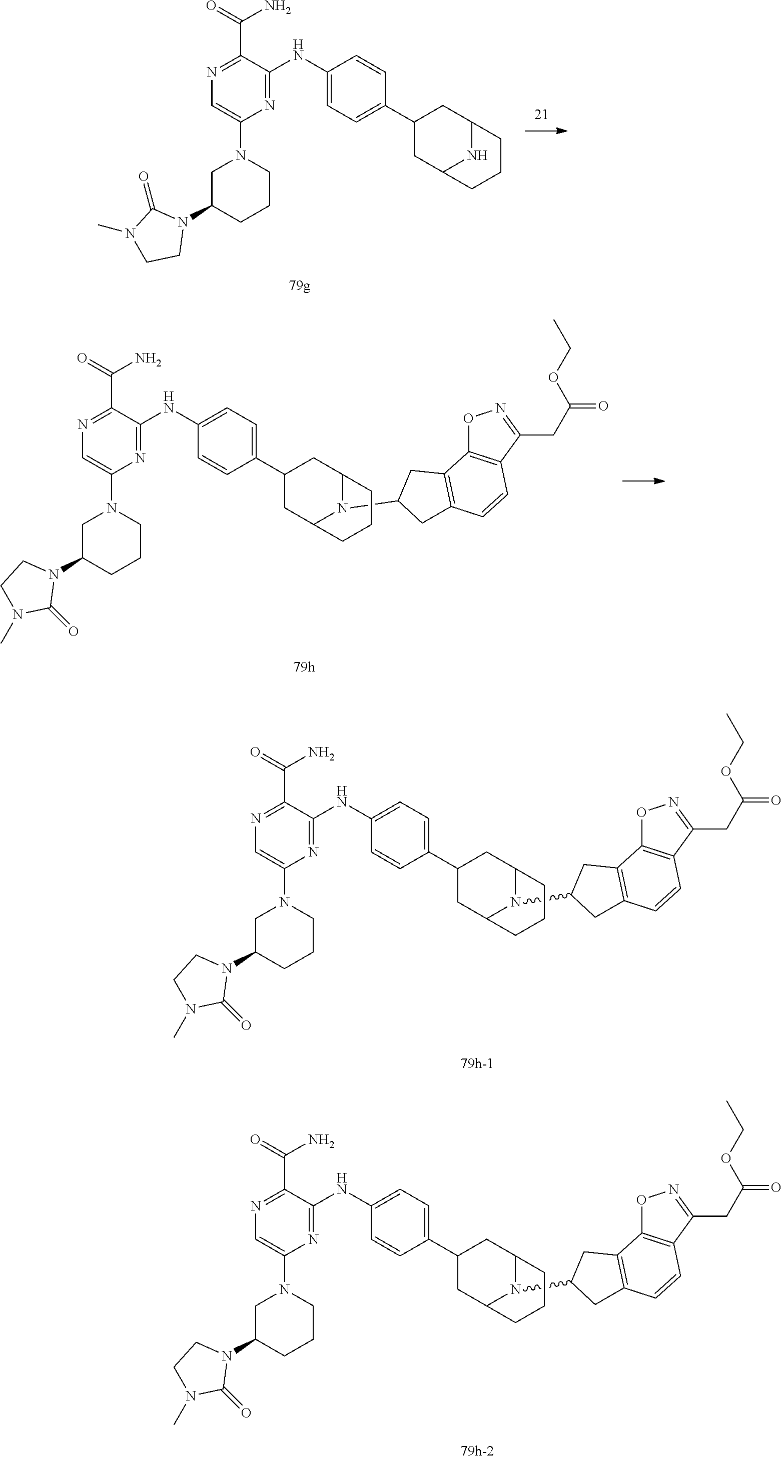 [SMILES: CN1CCN([C@@H]2CCCN(c3cnc(C(N)=O)c(Nc4ccc(C5CC6CCCC(C5)N6)cc4)n3)C2)C1=O ; CCOC(=O)Cc1noc2c3c(ccc12)CC(N1C2CCCC1CC(c1ccc(Nc4nc(N5CCC[C@@H](N6CCN(C)C6=O)C5)cnc4C(N)=O)cc1)C2)C3 ; CCOC(=O)Cc1noc2c3c(ccc12)CC(N1C2CCCC1CC(c1ccc(Nc4nc(N5CCC[C@@H](N6CCN(C)C6=O)C5)cnc4C(N)=O)cc1)C2)C3 ; CCOC(=O)Cc1noc2c3c(ccc12)CC(N1C2CCCC1CC(c1ccc(Nc4nc(N5CCC[C@@H](N6CCN(C)C6=O)C5)cnc4C(N)=O)cc1)C2)C3]

### Step 1: Preparation of Intermediate 79b

Lithium bis(trimethylsilyl)amide (1 M, 16.3 mL) was slowly added dropwise to a solution of 79a (3 g) in THF (30 mL) at −78° C. under nitrogen atmosphere. After the dropwise addition was completed, the mixture was reacted at −78° C. for 1 h. A solution of N-phenylbis(trifluoromethanesulfonyl)imide (5.37 g) in THF (30 mL) was slowly added to the reaction solution. After the addition was completed, the mixture was warmed to room temperature and reacted. After the reaction was completed, 200 mL of petroleum ether was added to the system, and saturated brine was added for extraction. The organic phase was dried over anhydrous sodium sulfate, filtered, and concentrated to give 79b (9 g).

¹H NMR (500 MHz, DMSO-d6) δ 6.03 (d, J=5.9 Hz, 1H), 4.71 (d, J=26.5 Hz, 1H), 4.41 (d, J=21.2 Hz, 1H), 2.80-2.67 (m, 1H), 2.23 (d, J=18.0 Hz, 1H), 1.72-1.46 (m, 6H), 1.39 (s, 9H).

### Step 2: Preparation of Intermediate 79c

4-nitrophenylboronic acid (2.82 g), 79b (7 g), potassium carbonate (3.91 g), Pd(dppf)₂Cl₂ (0.7 g), 1,4-dioxane (50 mL), and water (10 mL) were added to a reaction flask in sequence, and the mixture was purged several times with nitrogen, heated to 90° C., and reacted. After the reaction was completed, about 200 mL of methyl tert-butyl ether and 200 mL of saturated brine were added for extraction. The organic phase was dried over anhydrous sodium sulfate, filtered, and concentrated to give a crude product, and the crude product was slurried with petroleum ether and filtered under vacuum to give 79c (4 g).

¹H NMR (500 MHz, DMSO-d6) δ 8.29-8.09 (m, 2H), 7.82-7.61 (m, 2H), 6.50 (dd, J=11.0, 5.7 Hz, 1H), 4.84-4.59 (m, 1H), 4.46 (dd, J=21.3, 7.3 Hz, 1H), 2.84 (ddt, J=17.8, 7.5, 1.7 Hz, 1H), 2.42 (d, J=17.8 Hz, 1H), 1.77-1.54 (m, 5H), 1.49 (dd, J=6.7, 3.2 Hz, 1H), 1.42 (d, J=1.6 Hz, 9H).

### Step 3: Preparation of Intermediate 79d

79c (4 g), 10% palladium on carbon (2 g), and methanol (30 mL) were added to a reaction flask in sequence, and the mixture was purged with hydrogen and reacted overnight at room temperature. After the reaction was completed, the reaction solution was filtered under vacuum, and the filtrate was concentrated to give 79d (2.3 g).

¹H NMR (500 MHz, DMSO-d6) δ 6.96-6.87 (m, 2H), 6.53-6.37 (m, 2H), 5.05 (s, 2H), 4.37-4.25 (m, 2H), 2.16 (tt, J=13.1, 5.3 Hz, 1H), 2.01 (dttt, J=13.3, 7.8, 5.2, 3.2 Hz, 3H), 1.56-1.34 (m, 16H).

### Step 4: Preparation of Intermediate 79e

Intermediate 1f (2.3 g), 79d (2.2 g), cesium carbonate (6.8 g), BINAP (0.4 g), palladium acetate (0.15 g), and 1,4-dioxane (20 mL) were added to a single-necked flask in sequence, and the mixture was heated to 100° C. and reacted under nitrogen atmosphere. After the reaction was completed, the reaction solution was cooled to room temperature, mixed with silica gel, and then purified by column chromatography to give 79e (2.58 g).

MS(ESI, [M+H]+) m/z: 601.5.

### Step 5: Preparation of Intermediate 79f

Intermediate 79e (3.2 g), DMSO (30 mL), and cesium carbonate (1.73 g) were added to a single-necked flask in sequence. 30 wt % hydrogen peroxide (2 mL) was added under an ice bath. After the mixture was stirred for 5 min, the ice bath was removed, and the mixture was reacted at room temperature. After the reaction was completed, 300 mL of a saturated sodium sulfite solution was added to the residue. The color was not changed as detected using a potassium iodide starch reagent, and then 300 mL of ethyl acetate was added for extraction. 100 mL of ethyl acetate was then added for 3 times of extraction. The organic phases were combined, dried over anhydrous sodium sulfate, filtered, and concentrated by evaporation at reduced pressure to remove the solvent. The crude product was separated by silica gel column chromatography to give the target intermediate 79f (4.3 g).

MS(ESI, [M+H]+) m/z: 619.44.

### Step 6: Preparation of Intermediate 79g

79f (3.5 g), dichloromethane (20 mL), and TFA (5 mL) were added to a single-necked flask in sequence, and the mixture was reacted at room temperature. After the reaction was completed, the system was added to 200 mL of a saturated sodium bicarbonate solution, and then 200 mL of dichloromethane was added for extraction. The organic phase was dried over anhydrous sodium sulfate, filtered, and concentrated by evaporation at reduced pressure to remove the solvent, thus giving 79g (2.5 g).

MS(ESI, [M+H]+) m/z: 519.39.

### Step 7: Preparation of Compounds 79h-1 and 79h-2

Intermediate 21 (300 mg), intermediate 79g (600 mg), 1,2-dichloroethane (10 mL), isopropanol (3 mL), and sodium cyanoborohydride (291 mg) were added to a reaction flask in sequence, and the mixture was reacted overnight at room temperature. After the reaction was completed, 5 mL of a saturated sodium bicarbonate solution was added to the reaction solution to neutralize acetic acid, and then 100 mL of dichloromethane and 100 mL of water were added for extraction. The organic phase was separated, and the aqueous phase was extracted twice with 50 mL of dichloromethane. The organic phases were combined, washed with saturated brine, dried over anhydrous sodium sulfate, and concentrated, and the residue was purified by silica gel column chromatography to give 79h. 79h was separated by high performance liquid chromatography to give intermediates 79h-1 (163 mg) and 79h-2 (155 mg) in sequence.

The conditions of preparative chromatography were as follows: instrument and preparative column: YMC high pressure preparative chromatograph was used, and the preparative column was REFLECTI-Cellulose-SC. Mobile phase system:ethanol-dichloromethane (1:7)/n-hexane-0.1% diethylamine, isocratic elution: ethanol-dichloromethane (1:7)/n-hexane-0.1% diethylamine=50/50. 79h-1,79h-2: MS(ESI, [M+H]⁺) m/z: 762.64.

### Step 8: Preparation of Compound 79

Intermediate 79h-1 (100 mg), acrylamide (7.46 mg) and tetrahydrofuran (5 mL) were added to a reaction flask in sequence, and the mixture was cooled to 0° C. under N₂ atmosphere. Potassium tert-butoxide (1 M, 0.079 mL) was added, and the mixture was reacted at 0° C. for 1 h. After the reaction was completed, the reaction solution was added dropwise to an icy saturated aqueous ammonium chloride solution, and 50 mL of ethyl acetate was added for extraction. The organic phase was dried over anhydrous sodium sulfate, filtered, and concentrated, and the residue was purified by silica gel column chromatography to give compound 79 (50 mg).

MS(ESI, [M+H]+) m/z: 787.50.

¹H NMR (500 MHz, DMSO-d6) δ 11.23 (s, 1H), 11.09 (s, 1H), 7.76 (d, J=3.0 Hz, 1H), 7.65 (d, J=7.6 Hz, 2H), 7.51 (d, J=8.0 Hz, 2H), 7.37-7.30 (m, 1H), 7.26 (t, J=6.0 Hz, 3H), 4.58 (dd, J=12.0, 5.0 Hz, 1H), 4.34 (dd, J=45.6, 12.7 Hz, 2H), 4.10 (s, 1H), 3.62 (tt, J=11.1, 4.0 Hz, 1H), 3.48-3.33 (m, 4H), 3.30-3.20 (m, 3H), 3.15-2.89 (m, 3H), 2.71 (s, 8H), 2.61 (dt, J=17.4, 4.2 Hz, 1H), 2.31-1.90 (m, 6H), 1.87-1.71 (m, 3H), 1.63-1.43 (m, 4H), 1.00 (s, 2H).

### Example 80: Synthesis of Compound 80

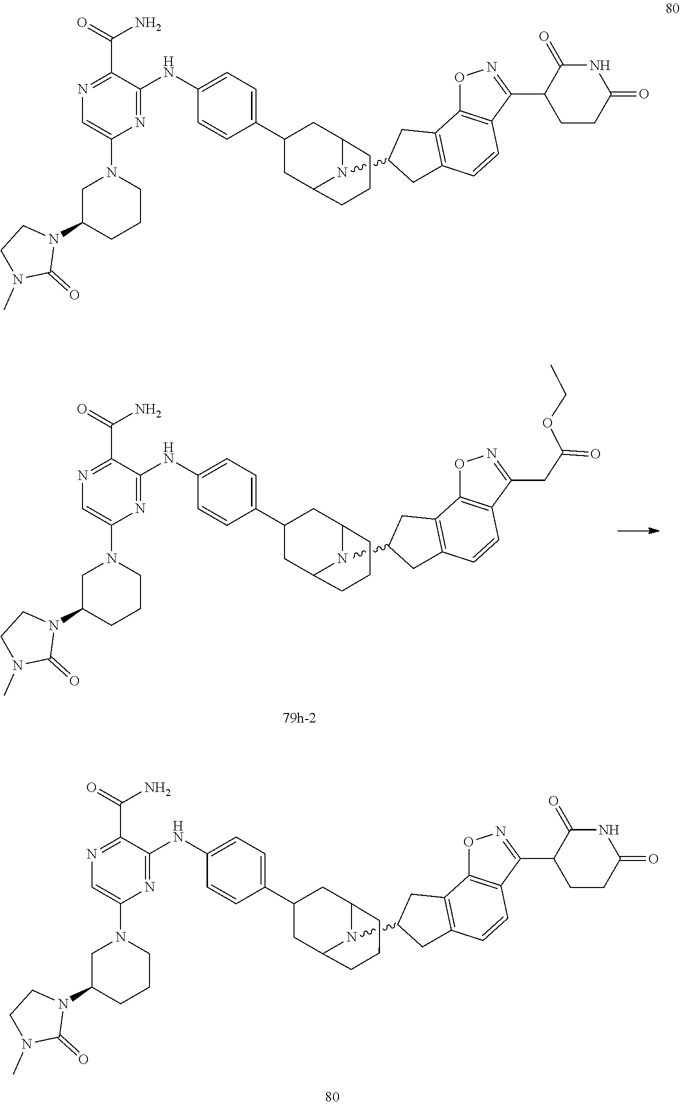 [SMILES: CN1CCN([C@@H]2CCCN(c3cnc(C(N)=O)c(Nc4ccc(C5CC6CCCC(C5)N6C5Cc6ccc7c(C8CCC(=O)NC8=O)noc7c6C5)cc4)n3)C2)C1=O ; CCOC(=O)Cc1noc2c3c(ccc12)CC(N1C2CCCC1CC(c1ccc(Nc4nc(N5CCC[C@@H](N6CCN(C)C6=O)C5)cnc4C(N)=O)cc1)C2)C3 ; CN1CCN([C@@H]2CCCN(c3cnc(C(N)=O)c(Nc4ccc(C5CC6CCCC(C5)N6C5Cc6ccc7c(C8CCC(=O)NC8=O)noc7c6C5)cc4)n3)C2)C1=O]

### Step 1: Preparation of Compound 80

Intermediate 79h-2 (100 mg), acrylamide (7.46 mg) and tetrahydrofuran (5 mL) were added to a reaction flask in sequence, and the mixture was cooled to 0° C. under N₂ atmosphere. Potassium tert-butoxide (1 M, 0.079 mL) was added, and the mixture was reacted at 0° C. After the reaction was completed, the reaction solution was added dropwise to an icy saturated aqueous ammonium chloride solution, and 50 mL of ethyl acetate was added for extraction. The organic phase was dried over anhydrous sodium sulfate, filtered, and concentrated, and the residue was purified by silica gel column chromatography to give compound 80 (45 mg).

MS(ESI, [M+H]+) m/z: 787.49.

¹H NMR (500 MHz, DMSO-d6) δ 11.23 (s, 1H), 11.09 (s, 1H), 7.76 (d, J=3.0 Hz, 1H), 7.65 (d, J=7.6 Hz, 2H), 7.51 (d, J=8.0 Hz, 2H), 7.37-7.30 (m, 1H), 7.26 (t, J=6.0 Hz, 3H), 4.58 (dd, J=12.0, 5.0 Hz, 1H), 4.34 (dd, J=45.6, 12.7 Hz, 2H), 4.10 (s, 1H), 3.62 (tt, J=11.1, 4.0 Hz, 1H), 3.48-3.33 (m, 4H), 3.30-3.20 (m, 3H), 3.15-2.89 (m, 3H), 2.71 (s, 8H), 2.61 (dt, J=17.4, 4.2 Hz, 1H), 2.31-1.90 (m, 6H), 1.87-1.71 (m, 3H), 1.63-1.43 (m, 4H), 1.00 (s, 2H).

### Example 81: Synthesis of Compound 81

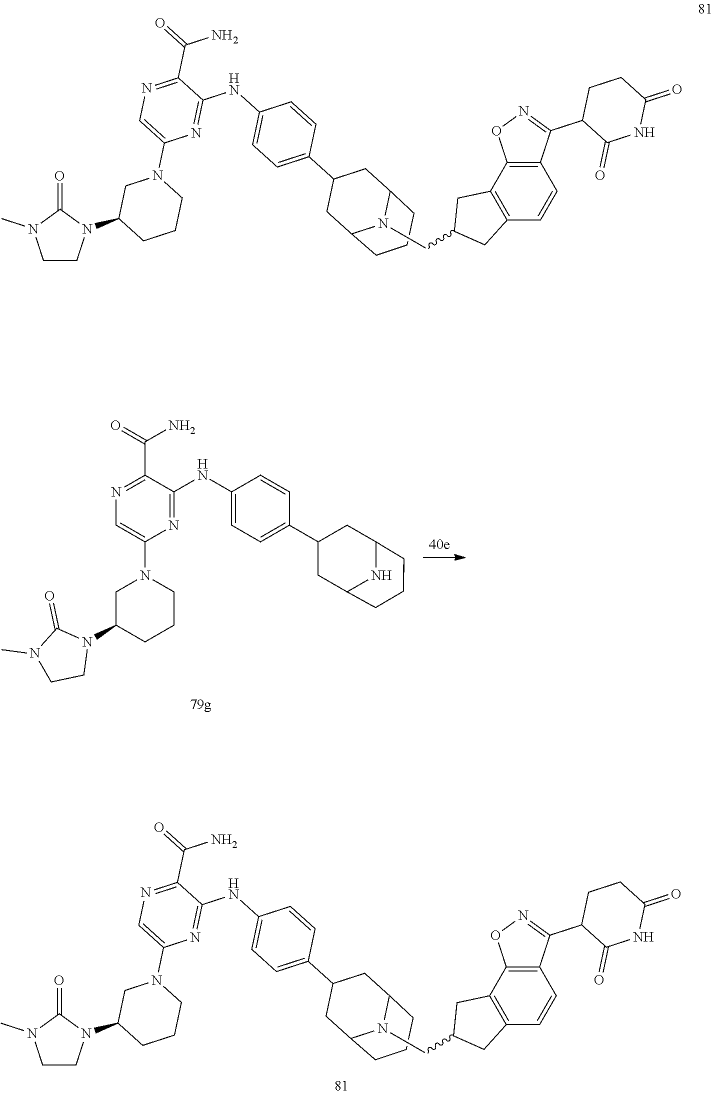 [SMILES: CN1CCN([C@@H]2CCCN(c3cnc(C(N)=O)c(Nc4ccc(C5CC6CCCC(C5)N6CC5Cc6ccc7c(C8CCC(=O)NC8=O)noc7c6C5)cc4)n3)C2)C1=O ; CN1CCN([C@@H]2CCCN(c3cnc(C(N)=O)c(Nc4ccc(C5CC6CCCC(C5)N6)cc4)n3)C2)C1=O ; CN1CCN([C@@H]2CCCN(c3cnc(C(N)=O)c(Nc4ccc(C5CC6CCCC(C5)N6CC5Cc6ccc7c(C8CCC(=O)NC8=O)noc7c6C5)cc4)n3)C2)C1=O]

### Step 1: Preparation of Compound 81

40e (100 mg), isopropanol (1 mL), 1,2-dichloroethane (5.00 mL), and 79g (190 mg) were added to a reaction flask in sequence, and sodium cyanoborohydride (63 mg) was added. The mixture was reacted at room temperature. After the reaction was completed, the reaction solution was mixed with silica gel and purified by column chromatography to give 81 (35 mg).

MS(ESI, [M+H]+) m/z: 801.63.

¹H NMR (500 MHz, DMSO-d6) δ 11.21 (s, 1H), 11.09 (s, 1H), 7.81-7.72 (m, 1H), 7.66 (s, 1H), 7.61 (d, J=8.0 Hz, 1H), 7.52 (d, J=8.1 Hz, 2H), 7.34 (s, 1H), 7.26 (dd, J=16.3, 8.1 Hz, 3H), 4.57 (dd, J=11.8, 5.0 Hz, 1H), 4.35 (dd, J=61.3, 12.9 Hz, 2H), 3.63 (td, J=10.9, 10.3, 5.5 Hz, 1H), 3.34 (d, J=7.2 Hz, 2H), 3.27 (dd, J=10.6, 7.1 Hz, 2H), 3.24-2.91 (m, 8H), 2.91-2.56 (m, 10H), 2.23-1.99 (m, 4H), 1.80 (dd, J=33.4, 12.5 Hz, 5H), 1.63-1.42 (m, 4H), 1.01 (d, J=12.4 Hz, 2H).

### Example 82: Synthesis of Compound 82

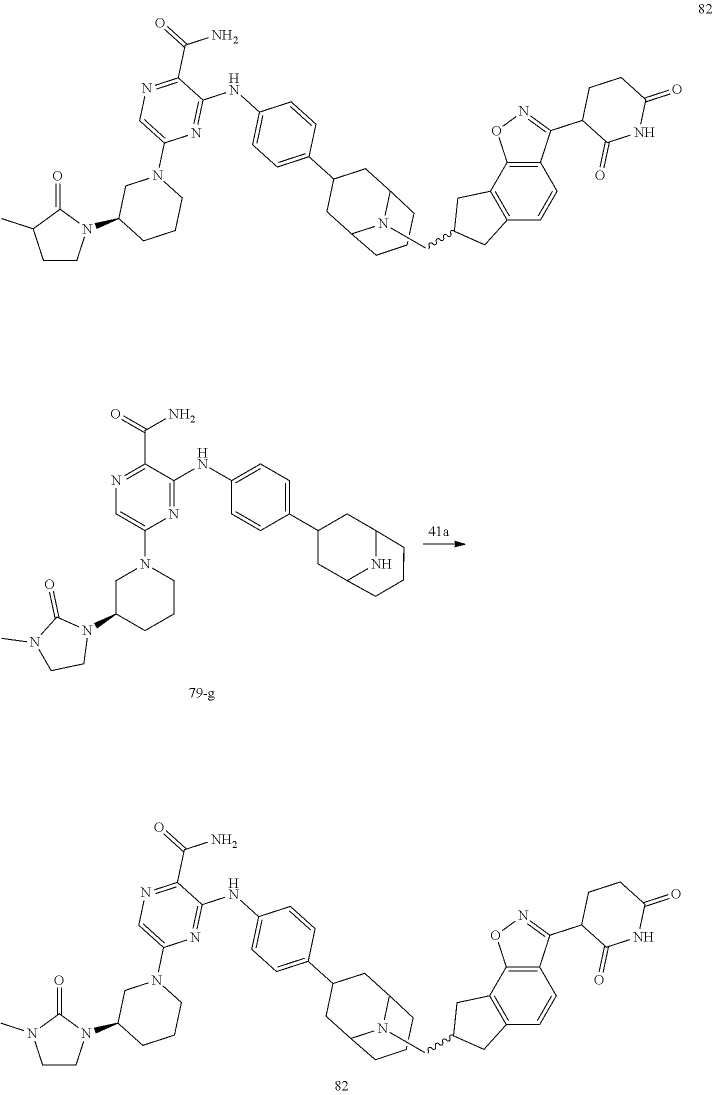 [SMILES: CC1CCN([C@@H]2CCCN(c3cnc(C(N)=O)c(Nc4ccc(C5CC6CCCC(C5)N6CC5Cc6ccc7c(C8CCC(=O)NC8=O)noc7c6C5)cc4)n3)C2)C1=O ; CN1CCN([C@@H]2CCCN(c3cnc(C(N)=O)c(Nc4ccc(C5CC6CCCC(C5)N6)cc4)n3)C2)C1=O ; CN1CCN([C@@H]2CCCN(c3cnc(C(N)=O)c(Nc4ccc(C5CC6CCCC(C5)N6CC5Cc6ccc7c(C8CCC(=O)NC8=O)noc7c6C5)cc4)n3)C2)C1=O]

### Step 1: Preparation of Compound 82

41a (100 mg), isopropanol (1 mL), 1,2-dichloroethane (5.00 mL), and 79 (190 mg) were added to a reaction flask in sequence, and sodium cyanoborohydride (18 mg) was added. The mixture was reacted at room temperature. After the reaction was completed, the reaction solution was mixed with silica gel and purified by column chromatography to give 82 (35 mg).

MS(ESI, [M+H]+) m/z: 801.51.

¹H NMR (500 MHz, DMSO-d6) δ 11.21 (s, 1H), 11.09 (s, 1H), 7.81-7.72 (m, 1H), 7.66 (s, 1H), 7.61 (d, J=8.0 Hz, 1H), 7.52 (d, J=8.1 Hz, 2H), 7.34 (s, 1H), 7.26 (dd, J=16.3, 8.1 Hz, 3H), 4.57 (dd, J=11.8, 5.0 Hz, 1H), 4.35 (dd, J=61.3, 12.9 Hz, 2H), 3.63 (td, J=10.9, 10.3, 5.5 Hz, 1H), 3.34 (d, J=7.2 Hz, 2H), 3.27 (dd, J=10.6, 7.1 Hz, 2H), 3.24-2.91 (m, 8H), 2.91-2.56 (m, 10H), 2.23-1.99 (m, 4H), 1.80 (dd, J=33.4, 12.5 Hz, 5H), 1.63-1.42 (m, 4H), 1.01 (d, J=12.4 Hz, 2H).

### Example 83: Preparation of Compound 83

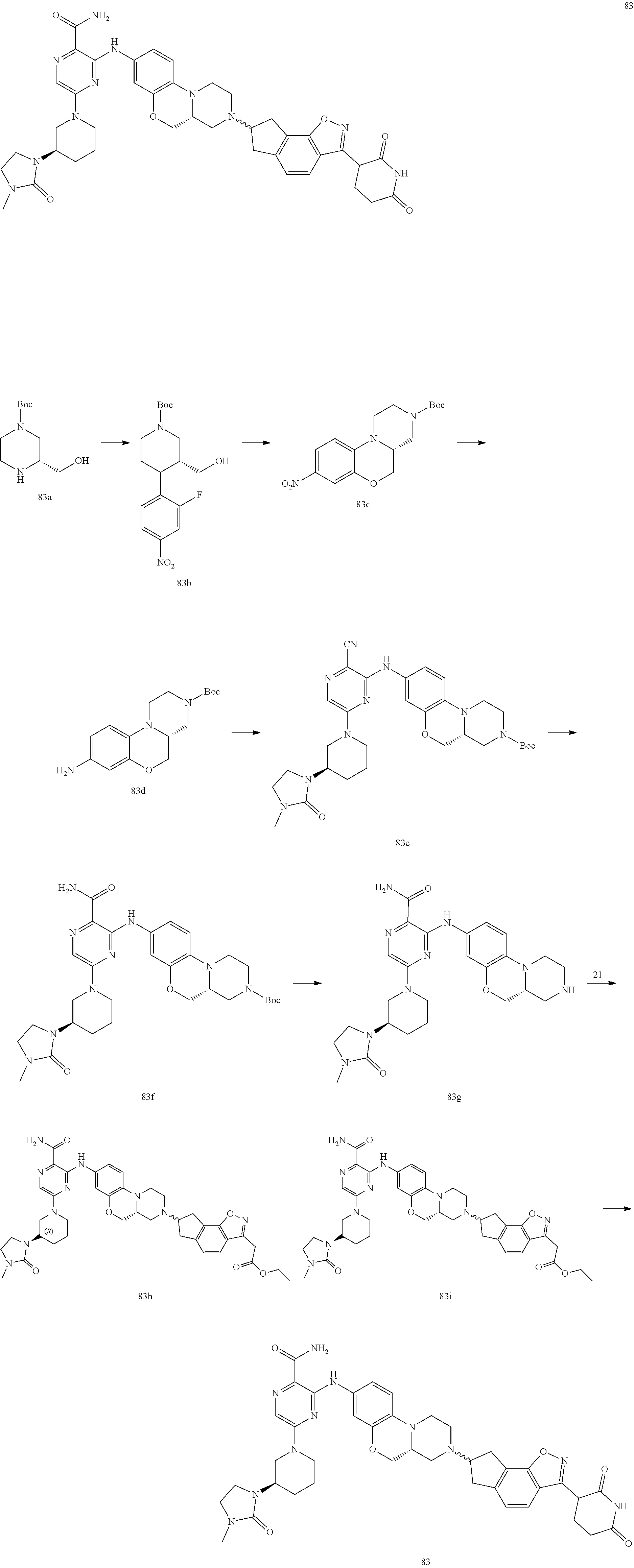

### Step 1: Preparation of Intermediate 83b

Intermediate 83a (2.7 g), 3,4-difluoronitrobenzene (2.0 g), potassium hydroxide (2.1 g), and dimethyl sulfoxide (25 mL) were added to a reaction flask in sequence, and the mixture was reacted at room temperature for 4 h. 60 mL of ethyl acetate and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 83b (3.4 g).

MS(ESI, [M+H]+) m/z: 356.1.

¹H NMR (500 MHz, DMSO-d6) δ 8.16 (dd, J=11.0, 2.8 Hz, 1H), 8.14-8.09 (m, 1H), 7.44 (t, J=8.8 Hz, 1H), 4.14 (d, J=6.1 Hz, 2H), 3.99 (d, J=6.1 Hz, 1H), 3.72 (d, J=12.6 Hz, 1H), 3.00-2.79 (m, 3H), 2.59 (tt, J=11.1, 4.6 Hz, 2H), 1.39 (s, 9H).

### Step 2: Preparation of Intermediate 833c

Intermediate 83b (2.0 g), potassium hydroxide (0.95 g), and dimethyl sulfoxide (25 mL) were added to a reaction flask in sequence, and the mixture was heated to 70° C. and reacted for 16 h. The reaction solution was cooled to room temperature, and 50 mL of ethyl acetate and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give intermediate 83c (2.1 g).

¹H NMR (500 MHz, DMSO-d6) δ 7.77 (dd, J=9.1, 2.6 Hz, 1H), 7.51 (d, J=2.6 Hz, 1H), 7.04 (d, J=9.2 Hz, 1H), 4.41 (dd, J=11.1, 3.2 Hz, 1H), 4.05-3.92 (m, 4H), 3.37 (ddd, J=14.9, 7.4, 3.5 Hz, 3H), 2.91 (td, J=12.2, 3.3 Hz, 1H), 1.43 (s, 9H).

### Step 3: Preparation of Intermediate 83d

Intermediate 83c (2.1 g), 10% palladium on carbon (1.0 g), and methanol (30 mL) were added to a reaction flask in sequence, and the mixture was purged with hydrogen and reacted overnight. The reaction solution was filtered under vacuum, and the filtrate was concentrated to give intermediate 83d (1.8 g).

MS(ESI, [M+H]+) m/z: 306.2.

### Step 4: Preparation of Intermediate 83e

1f (1.50 g), 83d (1.57 g), BINAP (0.29 g), cesium carbonate (4.57 g), palladium acetate (0.11 g), and 1,4-dioxane (40 mL) were added to a reaction flask in sequence, and the mixture was purged three times with nitrogen, heated to 100° C., and reacted for 4 h. The reaction solution was cooled to room temperature, and 50 mL of ethyl acetate and 100 mL of water were added to the residue. The organic phase was separated, washed with a saturated sodium chloride solution, dried over anhydrous sodium sulfate, filtered, and concentrated. The concentrate was separated and purified by silica gel column chromatography to give intermediate 83e (1.67 g).

MS(ESI, [M+H]+) m/z: 590.4.

1H NMR (500 MHz, DMSO-d6) δ 8.75 (s, 1H), 7.76 (s, 1H), 7.04 (dd, J=8.8, 2.5 Hz, 1H), 6.92 (d, J=2.4 Hz, 1H), 6.79 (d, J=8.9 Hz, 1H), 4.28 (dd, J=10.8, 2.7 Hz, 3H), 4.05-3.82 (m, 3H), 3.70 (d, J=11.9 Hz, 1H), 3.52 (td, J=10.3, 4.8 Hz, 1H), 3.30-3.20 (m, 4H), 3.07-2.85 (m, 4H), 2.67 (s, 3H), 2.62-2.52 (m, 1H), 2.48 (d, J=3.4 Hz, 2H), 1.82-1.70 (m, 3H), 1.42 (s, 9H).

### Step 5: Preparation of Intermediate 83f

Intermediate 83e (0.90 g), cesium carbonate (0.50 g), water (5 mL), DMSO (20 mL), and methanol (5 mL) were added to a reaction flask in sequence. The reaction solution was cooled to 0° C., and 30 wt % hydrogen peroxide (0.52 g) was added dropwise. After the dropwise addition was completed, the mixture was allowed to react at room temperature for 1 h. 50 mL of water was added to the reaction solution, and then a saturated aqueous sodium sulfite solution was added to neutralize hydrogen peroxide. The mixture was stirred for 10 min and filtered, and the filter cake was collected and dried to give intermediate 8m (0.85 g).

MS(ESI, [M+H]⁺) m/z: 608.51.

### Step 6: Preparation of Intermediate 83g

Intermediate 83f (0.85 g) and trifluoroacetic acid (15 mL) were added to a reaction flask in sequence, and the mixture was reacted at room temperature for 2 h. The pH was adjusted to about 8 with a saturated aqueous sodium bicarbonate solution. The mixture was stirred for 10 min and filtered, and the filter cake was collected and dried to give intermediate 83g (0.70 g).

MS(ESI, [M+H]+) m/z: 508.4.

1H NMR (500 MHz, DMSO-d6) δ 11.05 (s, 1H), 7.73 (d, J=2.9 Hz, 1H), 7.61 (s, 1H), 7.30 (d, J=2.8 Hz, 1H), 7.07 (d, J=2.4 Hz, 1H), 6.98 (dd, J=8.8, 2.5 Hz, 1H), 6.80 (d, J=8.9 Hz, 1H), 4.30 (d, J=13.0 Hz, 2H), 4.20 (dd, J=10.7, 2.7 Hz, 1H), 3.89 (dd, J=10.7, 8.7 Hz, 1H), 3.67 (d, J=11.9 Hz, 1H), 3.56 (tt, J=10.3, 4.2 Hz, 1H), 3.36-3.29 (m, 3H), 3.25 (dt, J=8.3, 2.9 Hz, 2H), 3.19-2.98 (m, 5H), 2.97-2.82 (m, 2H), 2.67 (s, 3H), 2.60 (td, J=12.0, 3.1 Hz, 1H), 1.79 (q, J=6.7, 4.9 Hz, 3H), 1.60-1.50 (m, 1H).

### Step 6: Preparation of Intermediates 83h and 831

Intermediate 83g (0.44 g), 21 (206 mg), dichloroethane/isopropanol (5:1, 25 mL), and glacial acetic acid (10 mg) were added to a reaction flask in sequence. After the mixture was stirred at room temperature for 30 min, sodium cyanoborohydride (0.11 g) was added, and the mixture was reacted at room temperature for 2 h. 50 mL of DCM and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give a mixture of intermediates 83h and 831 (0.32 g), which was subjected to resolution by preparative chromatography to give the prepeak 83h (0.13 g) and the postpeak 831 (0.14 g) in sequence.

The conditions of preparative chromatography were as follows: instrument and preparative column: YMC high pressure preparative chromatograph was used, and the preparative column was REGIS Cellulose-C. Mobile phase system: dichloromethane/n-hexane/ethanol; isocratic elution: dichloromethane/n-hexane/ethanol=45/40/15.

MS(ESI, [M+H]⁺) m/z: 751.6.

### Step 7: Preparation of Compound 83

Intermediate 831 (120 mg) and acrylamide (22 mg) were added to a reaction flask in sequence, and the mixture was cooled to 0° C. A solution of potassium tert-butoxide in tetrahydrofuran (0.25 mL, 1 M) was added dropwise, and the mixture was reacted at 0° C. After the reaction was completed, as confirmed by TLC, the reaction solution was poured into 40 mL of a cold aqueous ammonium chloride solution, and dichloroethane (40 mL each time) was added for three times of extraction. The organic phases were combined, washed with water and saturated sodium chloride, dried over anhydrous sodium sulfate, and filtered, and the concentrate was separated and purified by silica gel column chromatography to give compound 83 (20 mg).

MS(ESI, [M+H]+) m/z: 776.4.

¹H NMR (500 MHz, DMSO-d6) δ 11.09 (s, 1H), 11.04 (s, 1H), 7.73 (d, J=2.9 Hz, 1H), 7.67-7.58 (m, 2H), 7.32-7.24 (m, 2H), 7.05 (d, J=2.4 Hz, 1H), 6.98 (dd, J=8.7, 2.5 Hz, 1H), 6.79 (d, J=9.0 Hz, 1H), 4.57 (dd, J=11.9, 5.0 Hz, 1H), 4.33-4.20 (m, 3H), 3.91 (dd, J=10.6, 8.9 Hz, 1H), 3.68 (d, J=11.3 Hz, 1H), 3.55 (dq, J=10.5, 5.9 Hz, 1H), 3.45 (q, J=7.7 Hz, 1H), 3.28-3.15 (m, 4H), 3.12-3.00 (m, 5H), 3.01-2.86 (m, 3H), 2.77 (ddd, J=17.2, 11.9, 5.4 Hz, 1H), 2.68 (s, 3H), 2.61 (ddd, J=13.4, 7.2, 3.4 Hz, 2H), 2.53 (d, J=5.6 Hz, 1H), 2.47 (d, J=8.3 Hz, 1H), 2.29 (dd, J=12.2, 9.0 Hz, 1H), 2.19 (dt, J=13.8, 5.4 Hz, 1H), 1.89 (t, J=10.5 Hz, 1H), 1.78 (dq, J=10.8, 5.8, 3.2 Hz, 3H), 1.55 (d, J=12.4 Hz, 1H).

### Example 84: Synthesis of Compound 84

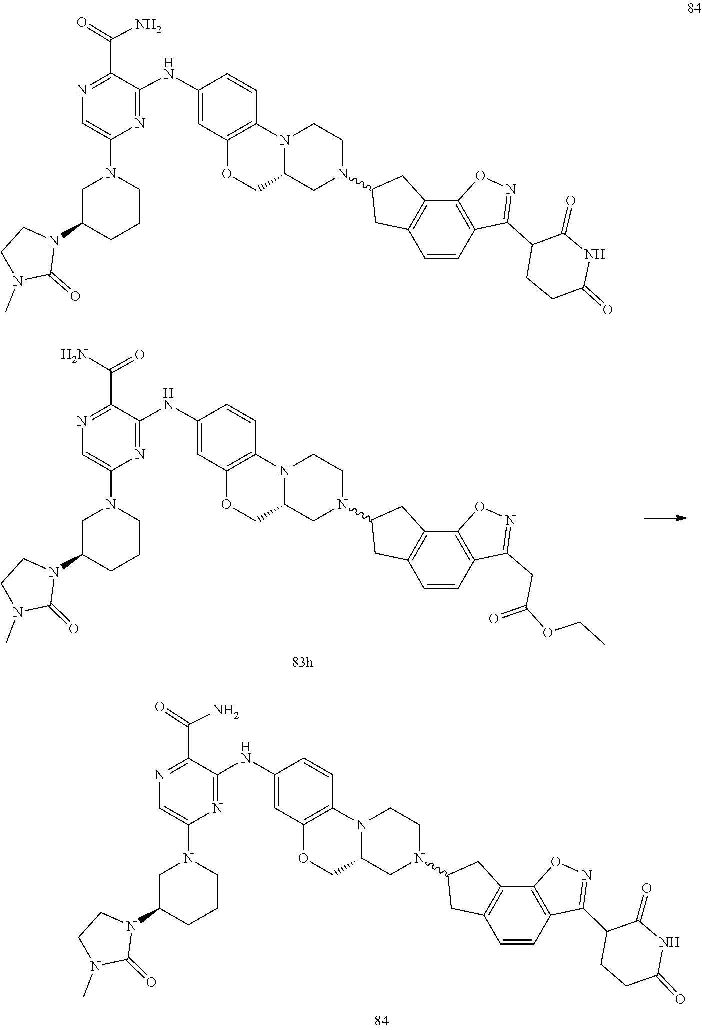 [SMILES: CN1CCN([C@@H]2CCCN(c3cnc(C(N)=O)c(Nc4ccc5c(c4)OC[C@H]4CN(C6Cc7ccc8c(C9CCC(=O)NC9=O)noc8c7C6)CCN54)n3)C2)C1=O ; CCOC(=O)Cc1noc2c3c(ccc12)CC(N1CCN2c4ccc(Nc5nc(N6CCC[C@@H](N7CCN(C)C7=O)C6)cnc5C(N)=O)cc4OC[C@H]2C1)C3 ; CN1CCN([C@@H]2CCCN(c3cnc(C(N)=O)c(Nc4ccc5c(c4)OC[C@H]4CN(C6Cc7ccc8c(C9CCC(=O)NC9=O)noc8c7C6)CCN54)n3)C2)C1=O]

### Step 1: Preparation of Compound 84

Intermediate 83h (130 mg) and acrylamide (22 mg) were added to a reaction flask in sequence, and the mixture was cooled to 0° C. A solution of potassium tert-butoxide in tetrahydrofuran (0.26 mL, 1 M) was added dropwise, and the mixture was reacted at 0° C. After the reaction was completed, as confirmed by TLC, the reaction solution was poured into 40 mL of a cold aqueous ammonium chloride solution, and dichloroethane (40 mL each time) was added for three times of extraction. The organic phases were combined, washed with water and saturated sodium chloride, dried over anhydrous sodium sulfate, and filtered, and the concentrate was separated and purified by silica gel column chromatography to give compound 84 (18 mg).

MS(ESI, [M+H]⁺) m/z: 776.4.

1H NMR (500 MHz, DMSO-d6) δ 11.09 (s, 1H), 11.04 (s, 1H), 7.77-7.69 (m, 1H), 7.68-7.58 (m, 2H), 7.33-7.24 (m, 2H), 7.06 (d, J=2.5 Hz, 1H), 6.97 (dd, J=8.8, 2.5 Hz, 1H), 6.79 (d, J=8.8 Hz, 1H), 4.57 (dd, J=11.9, 5.0 Hz, 1H), 4.34-4.21 (m, 3H), 3.92 (t, J=9.7 Hz, 1H), 3.68 (d, J=11.2 Hz, 1H), 3.55 (td, J=10.5, 10.1, 5.7 Hz, 1H), 3.42 (t, J=7.7 Hz, 1H), 3.36 (d, J=5.3 Hz, 2H), 3.28-3.20 (m, 3H), 3.05 (dd, J=19.7, 11.7 Hz, 6H), 2.93 (t, J=12.7 Hz, 1H), 2.77 (ddd, J=17.2, 11.9, 5.3 Hz, 1H), 2.68 (s, 3H), 2.62 (td, J=12.9, 11.9, 7.0 Hz, 2H), 2.53 (d, J=6.9 Hz, 1H), 2.47 (d, J=9.5 Hz, 1H), 2.28 (t, J=10.7 Hz, 1H), 2.19 (q, J=8.0, 6.6 Hz, 1H), 1.89 (t, J=10.9 Hz, 1H), 1.79 (q, J=13.5, 10.7 Hz, 3H), 1.55 (d, J=11.6 Hz, 1H).

### Example 85: Synthesis of Compound 85

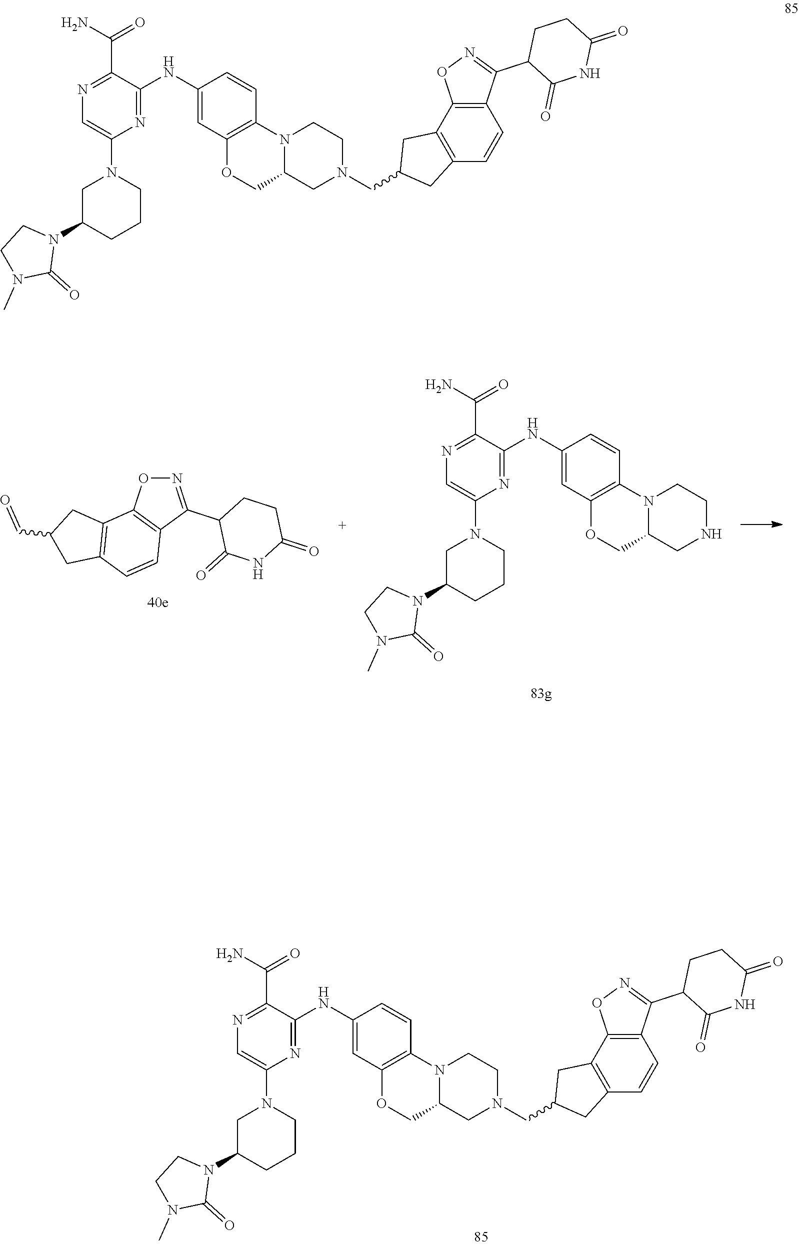

### Step 1: Preparation of Compound 85

Intermediate 83g (102 mg), 40e (60 mg), dichloroethane/isopropanol (5:1, 25 mL), and glacial acetic acid (10 mg) were added to a reaction flask in sequence. After the mixture was stirred at room temperature for 30 min, sodium cyanoborohydride (13 mg) was added, and the mixture was reacted at room temperature for 2 h. 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 85 (42 mg).

MS(ESI, [M+H]⁺) m/z: 790.4.

1H NMR (500 MHz, DMSO-d6) δ 11.08 (s, 1H), 11.03 (s, 1H), 7.73 (d, J=2.9 Hz, 1H), 7.61 (d, J=7.5 Hz, 2H), 7.32-7.25 (m, 2H), 7.05 (d, J=2.4 Hz, 1H), 6.98 (dd, J=8.8, 2.4 Hz, 1H), 6.79 (d, J=8.8 Hz, 1H), 4.57 (ddd, J=11.8, 4.9, 1.5 Hz, 1H), 4.29 (s, 2H), 4.22 (dd, J=10.6, 2.7 Hz, 1H), 3.94-3.87 (m, 1H), 3.65 (d, J=11.5 Hz, 1H), 3.59-3.52 (m, 1H), 3.34 (d, J=8.1 Hz, 3H), 3.24 (dd, J=9.1, 7.2 Hz, 3H), 3.17 (d, J=5.3 Hz, 1H), 3.08 (t, J=11.8 Hz, 1H), 3.04-2.98 (m, 2H), 2.98-2.88 (m, 4H), 2.84 (dd, J=16.2, 5.1 Hz, 1H), 2.77 (ddd, J=17.1, 11.9, 5.3 Hz, 1H), 2.67 (s, 3H), 2.64-2.57 (m, 2H), 2.40 (t, J=7.3 Hz, 2H), 2.22-2.13 (m, 2H), 1.78 (p, J=10.7, 9.8 Hz, 4H), 1.55 (d, J=14.9 Hz, 1H).

### Example 86: Synthesis of Compound 86

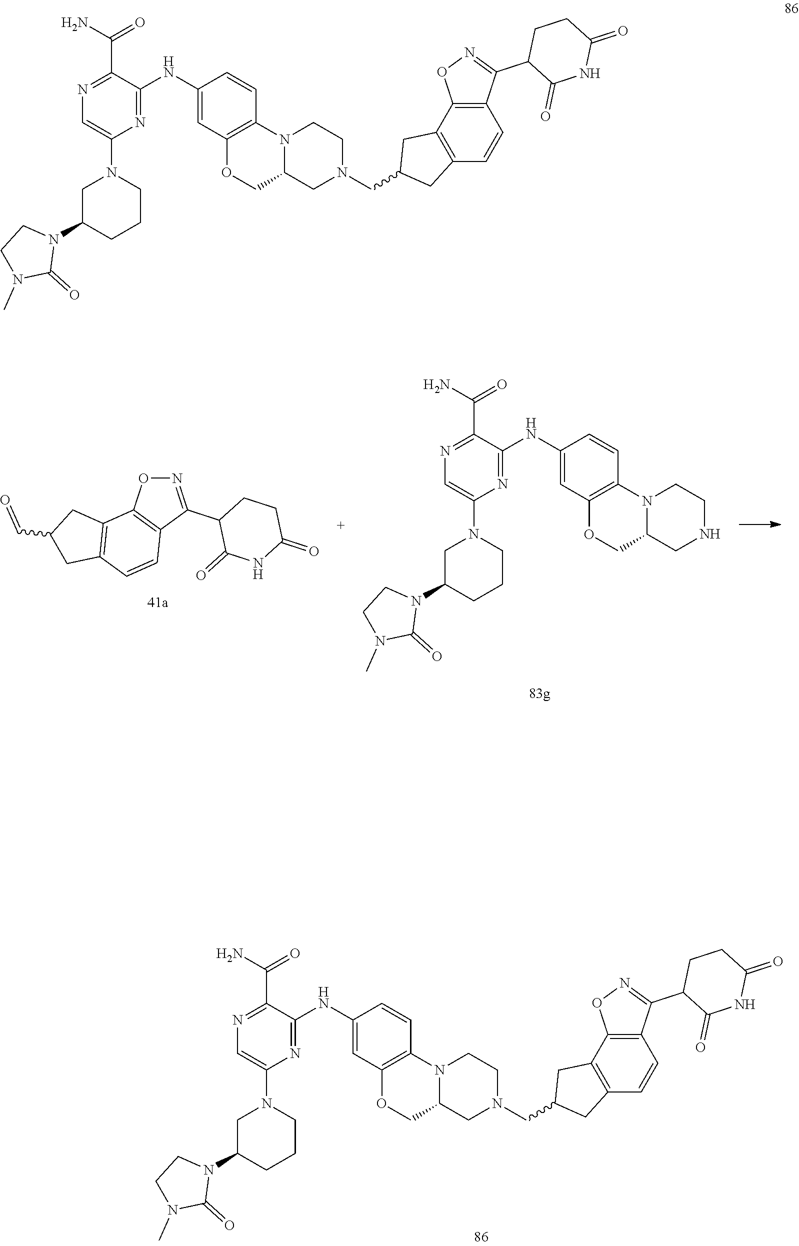

### Step 1: Preparation of Compound 86

Intermediate 83g (102 mg), 41a (60 mg), dichloroethane/isopropanol (5:1, 25 mL), and glacial acetic acid (10 mg) were added to a reaction flask in sequence. After the mixture was stirred at room temperature for 30 min, sodium cyanoborohydride (13 mg) was added, and the mixture was reacted at room temperature for 2 h. 50 mL of dichloromethane and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 86 (38 mg).

MS(ESI, [M+H]⁺) m/z: 790.4.

1H NMR (500 MHz, DMSO-d6) δ 11.08 (d, J=1.6 Hz, 1H), 11.03 (s, 1H), 7.73 (d, J=2.9 Hz, 1H), 7.61 (d, J=7.7 Hz, 2H), 7.32-7.24 (m, 2H), 7.05 (d, J=2.4 Hz, 1H), 6.97 (dd, J=8.8, 2.5 Hz, 1H), 6.79 (d, J=8.9 Hz, 1H), 4.57 (ddd, J=11.9, 5.0, 1.8 Hz, 1H), 4.29 (s, 2H), 4.21 (dd, J=10.5, 2.7 Hz, 1H), 3.90 (dd, J=10.7, 9.0 Hz, 1H), 3.65 (d, J=11.4 Hz, 1H), 3.59-3.52 (m, 1H), 3.34 (d, J=8.1 Hz, 3H), 3.28-3.22 (m, 3H), 3.20-3.15 (m, 1H), 3.08 (t, J=11.8 Hz, 1H), 3.01 (d, J=10.3 Hz, 2H), 2.93 (t, J=15.0 Hz, 4H), 2.84 (dd, J=16.0, 5.1 Hz, 1H), 2.77 (ddd, J=17.1, 11.9, 5.3 Hz, 1H), 2.67 (s, 3H), 2.65-2.57 (m, 2H), 2.40 (dd, J=7.6, 4.4 Hz, 2H), 2.17 (ddd, J=14.7, 10.1, 3.5 Hz, 2H), 1.77 (q, J=11.3, 10.4 Hz, 4H), 1.55 (d, J=11.3 Hz, 1H).

### Example 87: Synthesis of Compound 87

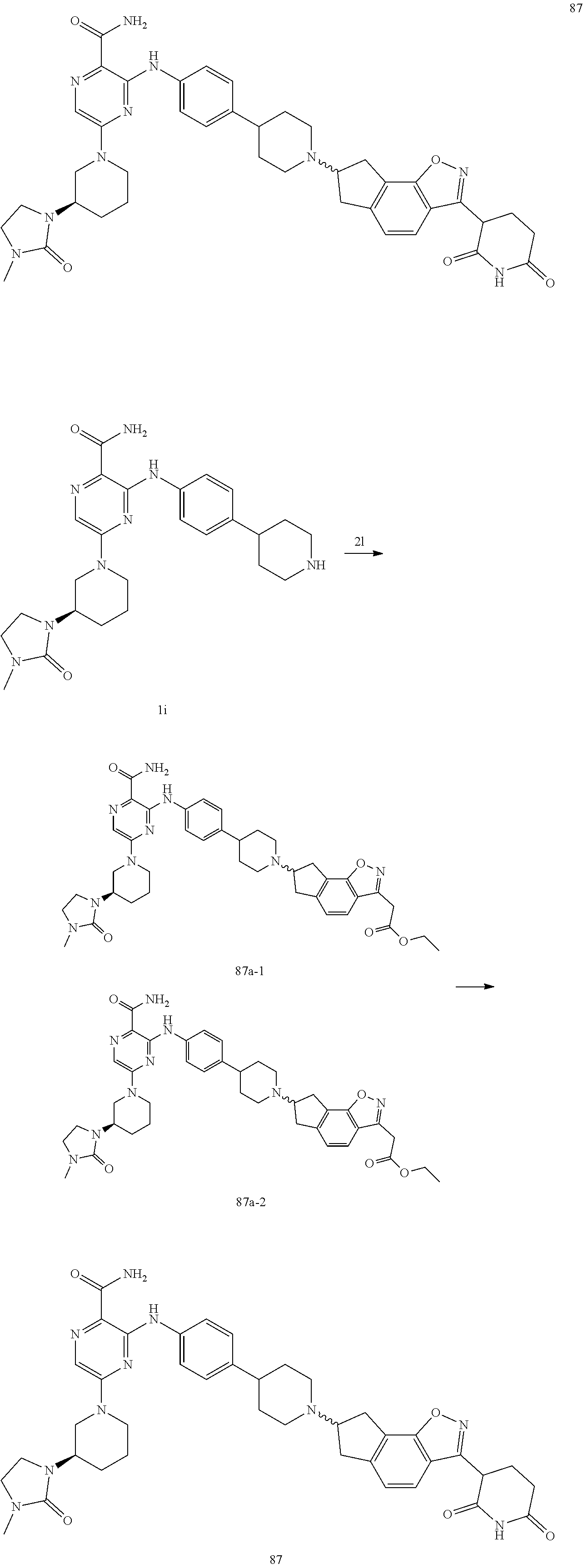 [SMILES: CN1CCN([C@@H]2CCCN(c3cnc(C(N)=O)c(Nc4ccc(C5CCN(C6Cc7ccc8c(C9CCC(=O)NC9=O)noc8c7C6)CC5)cc4)n3)C2)C1=O ; CN1CCN([C@@H]2CCCN(c3cnc(C(N)=O)c(Nc4ccc(C5CCNCC5)cc4)n3)C2)C1=O ; CCOC(=O)Cc1noc2c3c(ccc12)CC(N1CCC(c2ccc(Nc4nc(N5CCC[C@@H](N6CCN(C)C6=O)C5)cnc4C(N)=O)cc2)CC1)C3 ; CCOC(=O)Cc1noc2c3c(ccc12)CC(N1CCC(c2ccc(Nc4nc(N5CCC[C@@H](N6CCN(C)C6=O)C5)cnc4C(N)=O)cc2)CC1)C3 ; CN1CCN([C@@H]2CCCN(c3cnc(C(N)=O)c(Nc4ccc(C5CCN(C6Cc7ccc8c(C9CCC(=O)NC9=O)noc8c7C6)CC5)cc4)n3)C2)C1=O]

### Step 1: Preparation of Compounds 87a-1 and 87a-2

Intermediate 21 (300 mg), intermediate 1l (600 mg), 1,2-dichloroethane (10 mL), isopropanol (3 mL), and sodium cyanoborohydride (291 mg) were added to a reaction flask in sequence, and the mixture was reacted overnight at room temperature. After the reaction was completed, 5 mL of a saturated sodium bicarbonate solution was added to the reaction solution to neutralize acetic acid, and then 100 mL of dichloromethane and 100 mL of water were added for extraction. The organic phase was separated, and the aqueous phase was extracted twice with 50 mL of dichloromethane. The organic phases were combined, washed with saturated brine, dried over anhydrous sodium sulfate, and concentrated, and the residue was purified by silica gel column chromatography to give a racemic modification. The racemic modification was separated by high performance liquid chromatography to give intermediates 87a-1 (150 mg) and 87a-2 (130 mg) in sequence.

The conditions of preparative chromatography were as follows: instrument and preparative column: YMC high pressure preparative chromatograph was used, and the preparative column was (R,R)-Whelk-O1 (4.6×250 mm, 5 μm). Mobile phase system: ethanol-dichloromethane (3:2)/n-hexane-0.1% diethylamine, isocratic elution: ethanol-dichloromethane (3:2)/n-hexane-0.1% diethylamine=50/50.

87a-1,87a-2: MS(ESI, [M+H]+) m/z: 722.70.

### Step 2: Preparation of Compound 87

Intermediate 87a-1 (100 mg), acrylamide (15 mg) and tetrahydrofuran (5 mL) were added to a reaction flask in sequence, and the mixture was cooled to 0° C. under N₂ atmosphere. Potassium tert-butoxide (1 M, 0.079 mL) was added, and the mixture was reacted at 0° C. After the reaction was completed, the reaction solution was added dropwise to an icy saturated aqueous ammonium chloride solution, and 50 mL of ethyl acetate was added for extraction. The organic phase was dried over anhydrous sodium sulfate, filtered, and concentrated, and the residue was purified by silica gel column chromatography to give compound 87 (35 mg).

MS(ESI, [M+H]+) m/z: 747.61.

¹H NMR (500 MHz, DMSO-d6) δ 11.20 (s, 1H), 11.09 (s, 1H), 7.76 (d, J=2.8 Hz, 1H), 7.70-7.58 (m, 2H), 7.50 (d, J=8.3 Hz, 2H), 7.38-7.31 (m, 1H), 7.27 (d, J=8.1 Hz, 1H), 7.17 (d, J=8.3 Hz, 2H), 4.57 (dd, J=11.8, 5.0 Hz, 1H), 4.32 (dd, J=31.0, 12.8 Hz, 2H), 3.61 (dt, J=11.0, 6.1 Hz, 1H), 3.50-3.39 (m, 1H), 3.35 (d, J=6.9 Hz, 3H), 3.25 (ddt, J=26.3, 15.8, 7.7 Hz, 4H), 3.00 (dq, J=33.1, 12.2, 11.6 Hz, 6H), 2.84-2.67 (m, 4H), 2.61 (dt, J=17.4, 4.3 Hz, 1H), 2.46 (s, 1H), 2.26-2.09 (m, 3H), 1.88-1.49 (m, 8H).

### Example 88: Synthesis of Compound 88

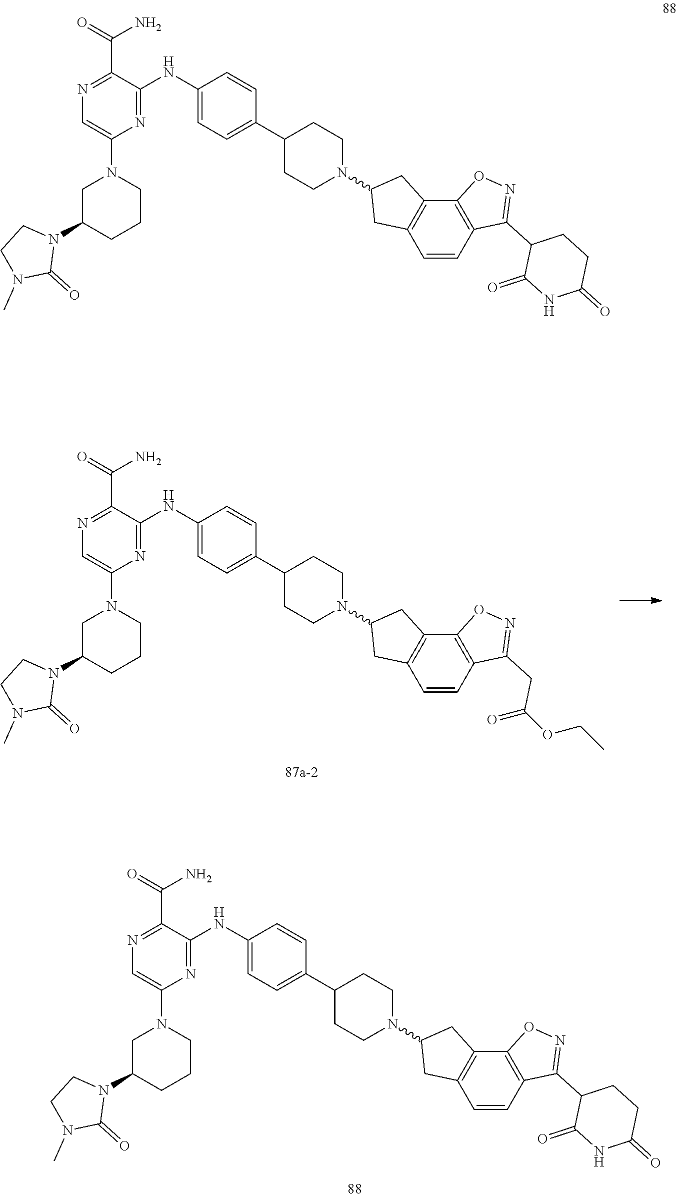 [SMILES: CN1CCN([C@@H]2CCCN(c3cnc(C(N)=O)c(Nc4ccc(C5CCN(C6Cc7ccc8c(C9CCC(=O)NC9=O)noc8c7C6)CC5)cc4)n3)C2)C1=O ; CCOC(=O)Cc1noc2c3c(ccc12)CC(N1CCC(c2ccc(Nc4nc(N5CCC[C@@H](N6CCN(C)C6=O)C5)cnc4C(N)=O)cc2)CC1)C3 ; CN1CCN([C@@H]2CCCN(c3cnc(C(N)=O)c(Nc4ccc(C5CCN(C6Cc7ccc8c(C9CCC(=O)NC9=O)noc8c7C6)CC5)cc4)n3)C2)C1=O]

### Step 1: Preparation of Compound 88

Intermediate 87a-2 (100 mg), acrylamide (15 mg), and tetrahydrofuran (5 mL) were added to a reaction flask in sequence, and the mixture was cooled to 0° C. under N₂ atmosphere. Potassium tert-butoxide (1 M, 0.079 mL) was added, and the mixture was reacted at 0° C. After the reaction was completed, the reaction solution was added dropwise to an icy saturated aqueous ammonium chloride solution, and 50 mL of ethyl acetate was added for extraction. The organic phase was dried over anhydrous sodium sulfate, filtered, and concentrated, and the residue was purified by silica gel column chromatography to give compound 88 (50 mg).

MS(ESI, [M+H]+) m/z: 747.55.

¹H NMR (500 MHz, DMSO-d6) δ 11.20 (s, 1H), 11.09 (s, 1H), 7.76 (d, J=2.8 Hz, 1H), 7.70-7.58 (m, 2H), 7.50 (d, J=8.3 Hz, 2H), 7.38-7.31 (m, 1H), 7.27 (d, J=8.1 Hz, 1H), 7.17 (d, J=8.3 Hz, 2H), 4.57 (dd, J=11.8, 5.0 Hz, 1H), 4.32 (dd, J=31.0, 12.8 Hz, 2H), 3.61 (dt, J=11.0, 6.1 Hz, 1H), 3.50-3.39 (m, 1H), 3.35 (d, J=6.9 Hz, 3H), 3.25 (ddt, J=26.3, 15.8, 7.7 Hz, 4H), 3.00 (dq, J=33.1, 12.2, 11.6 Hz, 6H), 2.84-2.67 (m, 4H), 2.61 (dt, J=17.4, 4.3 Hz, 1H), 2.46 (s, 1H), 2.26-2.09 (m, 3H), 1.88-1.49 (m, 8H).

### Example 89: Preparation of Compound 89

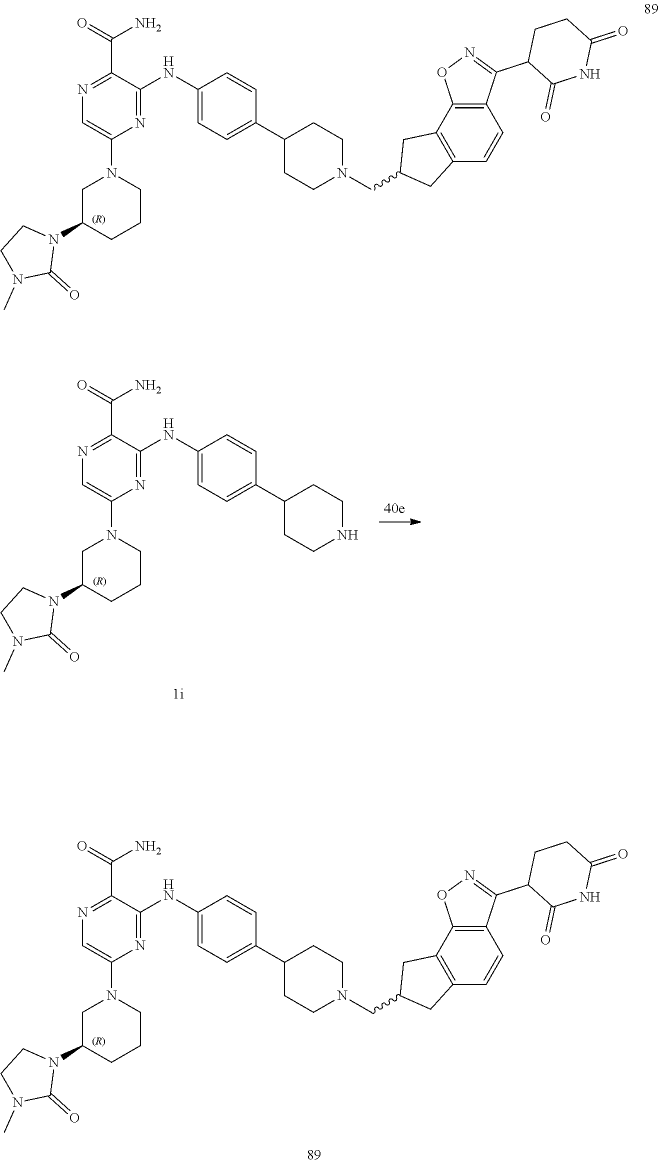

### Step 1: Preparation of Compound 89

Intermediate 1l (96 mg), 40e (60 mg), dichloroethane/isopropanol (5:1, 25 mL), and glacial acetic acid (10 mg) were added to a reaction flask in sequence. After the mixture was stirred at room temperature for 30 min, sodium cyanoborohydride (25.3 mg) was added, and the mixture was reacted at room temperature for 2 h. 50 mL of DCM and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 89 (25 mg).

MS(ESI, [M+H]⁺) m/z: 761.6.

1H NMR (500 MHz, DMSO-d6) δ 11.18 (s, 1H), 11.08 (s, 1H), 7.75 (s, 1H), 7.66 (s, 1H), 7.61 (d, J=7.9 Hz, 1H), 7.50 (d, J=8.0 Hz, 2H), 7.32 (s, 1H), 7.27 (d, J=8.1 Hz, 1H), 7.18 (d, J=8.2 Hz, 2H), 4.56 (dd, J=11.5, 4.9 Hz, 1H), 4.37 (d, J=12.1 Hz, 1H), 4.29 (d, J=12.9 Hz, 1H), 3.62 (d, J=12.6 Hz, 1H), 3.35 (s, 2H), 3.26 (d, J=10.2 Hz, 4H), 3.24-3.12 (m, 2H), 3.05-2.97 (m, 3H), 2.97-2.81 (m, 4H), 2.74 (s, 3H), 2.63 (s, 1H), 2.44 (s, 1H), 2.36 (d, J=7.1 Hz, 2H), 2.24-2.14 (m, 1H), 2.03 (t, J=11.4 Hz, 2H), 1.87-1.72 (m, 5H), 1.65 (d, J=13.4 Hz, 2H), 1.57 (s, 1H).

### Example 90: Preparation of Compound 90

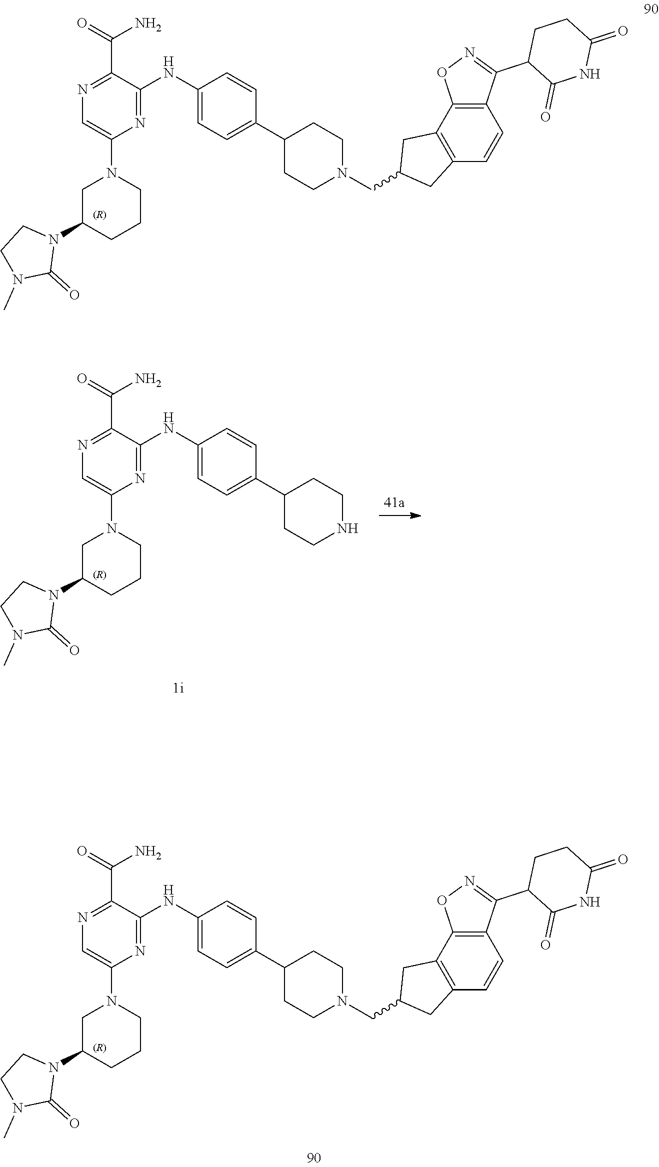

### Step 1: Preparation of Compound 90

Intermediate 1l (110 mg), 41a (65 mg), dichloroethane/isopropanol (5:1, 25 mL), and glacial acetic acid (10 mg) were added to a reaction flask in sequence. After the mixture was stirred at room temperature for 30 min, sodium cyanoborohydride (27.6 mg) was added, and the mixture was reacted at room temperature for 2 h. 50 mL of DCM and 100 mL of water were added to the reaction solution. The organic phase was separated, washed with a saturated sodium chloride solution, dried over anhydrous sodium sulfate, filtered, and concentrated. The concentrate was separated and purified by silica gel column chromatography to give compound 90 (31 mg).

MS(ESI, [M+H]⁺) m/z: 761.6.

¹H NMR (500 MHz, DMSO-d6) δ 11.18 (s, 1H), 11.08 (s, 1H), 7.75 (d, J=2.8 Hz, 1H), 7.66 (s, 1H), 7.61 (d, J=8.0 Hz, 1H), 7.53-7.47 (m, 2H), 7.32 (d, J=2.9 Hz, 1H), 7.27 (d, J=8.1 Hz, 1H), 7.18 (d, J=8.2 Hz, 2H), 4.57 (ddd, J=11.8, 5.0, 1.5 Hz, 1H), 4.37 (d, J=12.5 Hz, 1H), 4.29 (d, J=13.3 Hz, 1H), 3.62 (dq, J=8.2, 5.5, 4.1 Hz, 1H), 3.38-3.32 (m, 2H), 3.31-3.24 (m, 4H), 3.24-3.14 (m, 2H), 3.01 (dd, J=14.6, 6.2 Hz, 3H), 2.97-2.82 (m, 4H), 2.75 (s, 3H), 2.64-2.57 (m, 1H), 2.47-2.41 (m, 1H), 2.36 (d, J=7.3 Hz, 2H), 2.19 (dq, J=8.6, 4.4, 3.8 Hz, 1H), 2.03 (t, J=11.4 Hz, 2H), 1.86-1.72 (m, 5H), 1.66 (dd, J=16.9, 7.3 Hz, 2H), 1.57 (q, J=12.2 Hz, 1H).

### Example 91: Synthesis of Compound 91

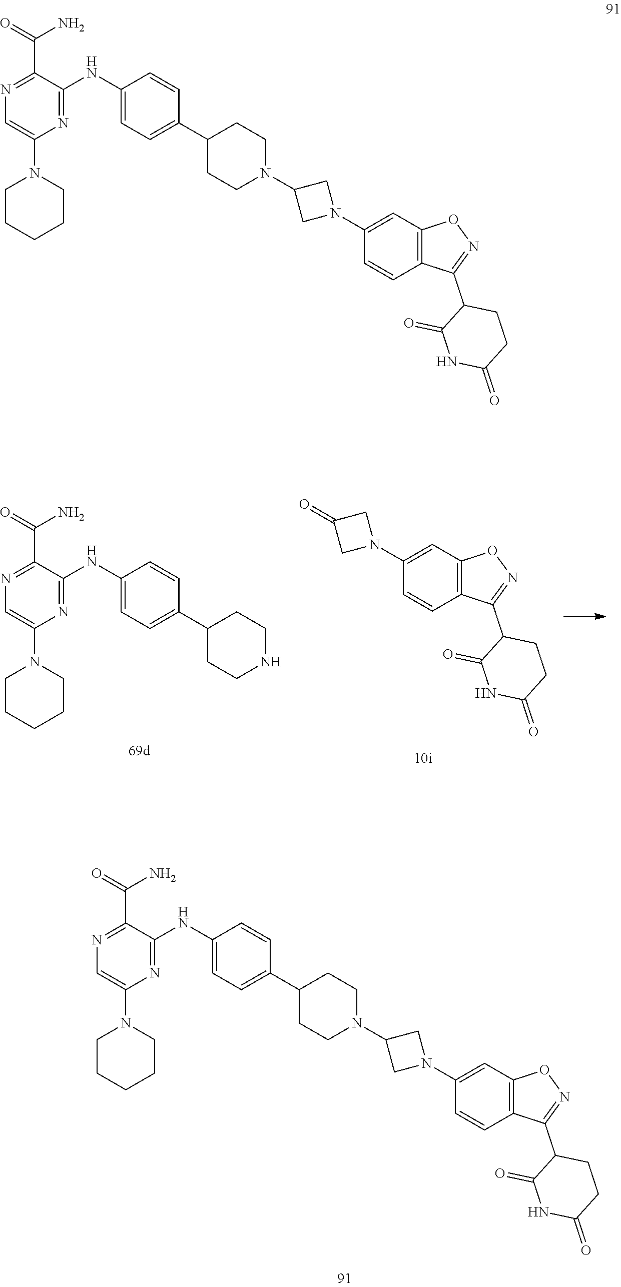 [SMILES: NC(=O)c1ncc(N2CCCCC2)nc1Nc1ccc(C2CCN(C3CN(c4ccc5c(C6CCC(=O)NC6=O)noc5c4)C3)CC2)cc1 ; NC(=O)c1ncc(N2CCCCC2)nc1Nc1ccc(C2CCNCC2)cc1 ; O=C1CN(c2ccc3c(C4CCC(=O)NC4=O)noc3c2)C1 ; NC(=O)c1ncc(N2CCCCC2)nc1Nc1ccc(C2CCN(C3CN(c4ccc5c(C6CCC(=O)NC6=O)noc5c4)C3)CC2)cc1]

### Step 1: Preparation of Compound 91

Intermediate 69d (150 mg), intermediate 101 (177 mg), dichloroethane (10 mL), isopropanol (2 mL), and glacial acetic acid (11.84 mg, 0.011 mL) were added to a reaction flask. After the mixture was stirred at room temperature for 30 min, sodium cyanoborohydride (74.3 mg) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give compound 91 (50 mg).

Q-TOF (ESI, [M+H]⁺) m/z: 664.3371

¹H NMR (500 MHz, DMSO-d₆) δ 11.28 (s, 1H), 11.04 (s, 1H), 7.73 (s, 1H), 7.66 (s, 1H), 7.56 (d, J=8.6 Hz, 1H), 7.51 (d, J=8.1 Hz, 2H), 7.31 (s, 1H), 7.18 (d, J=8.1 Hz, 2H), 6.56-6.47 (m, 2H), 4.44 (dd, J=11.4, 5.0 Hz, 1H), 4.04 (t, J=7.4 Hz, 2H), 3.73 (t, J=6.5 Hz, 2H), 3.67 (t, J=5.2 Hz, 4H), 2.93 (d, J=10.5 Hz, 2H), 2.74 (ddd, J=17.1, 11.5, 5.3 Hz, 1H), 2.58 (dt, J=17.4, 4.5 Hz, 1H), 2.43 (ddt, J=16.1, 11.8, 4.2 Hz, 2H), 2.22-2.13 (m, 1H), 1.96 (t, J=11.4 Hz, 2H), 1.78 (d, J=12.4 Hz, 2H), 1.68-1.62 (m, 3H), 1.62-1.55 (m, 5H).

### Example 92: Synthesis of Compound 92

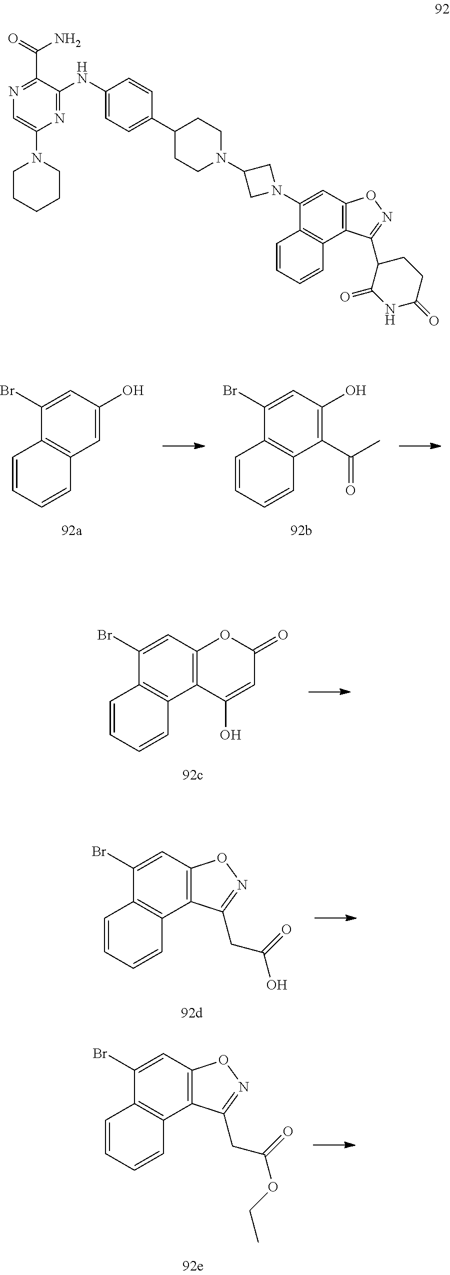 [SMILES: NC(=O)c1ncc(N2CCCCC2)nc1Nc1ccc(C2CCN(C3CN(c4cc5onc(C6CCC(=O)NC6=O)c5c5ccccc45)C3)CC2)cc1 ; Oc1cc(Br)c2ccccc2c1 ; CC(=O)c1c(O)cc(Br)c2ccccc12 ; O=c1cc(O)c2c(cc(Br)c3ccccc32)o1 ; O=C(O)Cc1noc2cc(Br)c3ccccc3c12 ; CCOC(=O)Cc1noc2cc(Br)c3ccccc3c12]

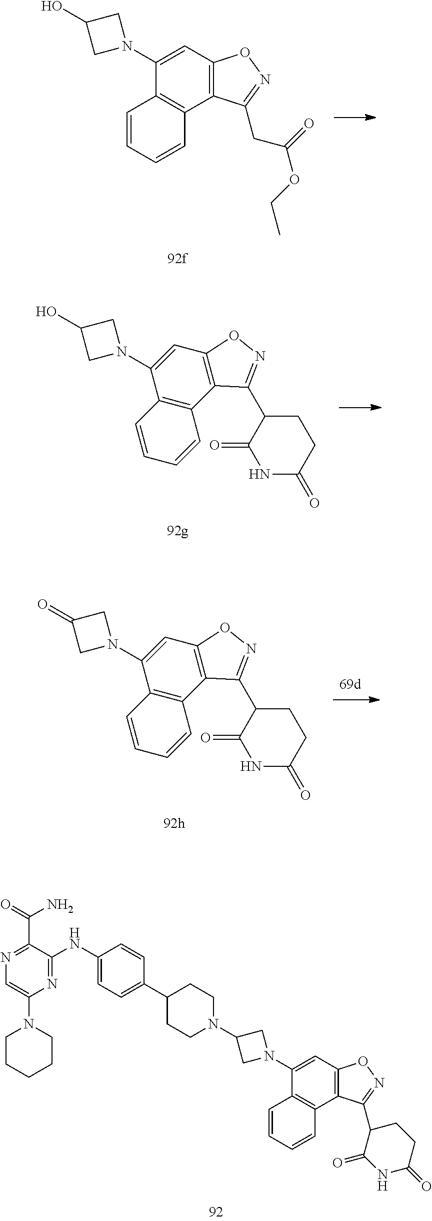 [SMILES: CCOC(=O)Cc1noc2cc(N3CC(O)C3)c3ccccc3c12 ; O=C1CCC(c2noc3cc(N4CC(O)C4)c4ccccc4c23)C(=O)N1 ; O=C1CN(c2cc3onc(C4CCC(=O)NC4=O)c3c3ccccc23)C1 ; NC(=O)c1ncc(N2CCCCC2)nc1Nc1ccc(C2CCN(C3CN(c4cc5onc(C6CCC(=O)NC6=O)c5c5ccccc45)C3)CC2)cc1]

### Step 1: Preparation of Intermediate 92b

Intermediate 92a (20 g), 1,2-dichloroethane (200 mL), acetyl chloride (7.04 g), and aluminum trichloride (17.93 g) were added to a reaction flask in sequence, and the mixture was heated to 85° C. and reacted. After the reaction was completed, as confirmed by TLC, the reaction solution was cooled to room temperature, slowly added to 1 M diluted hydrochloric acid with ice, and extracted with 200 mL dichloromethane. The organic phase was separated, and the aqueous phase was extracted twice with dichloromethane. The organic phases were combined, dried over anhydrous sodium sulfate, and filtered, and the filtrate was concentrated by evaporation at reduced pressure to remove the solvent. The residue was separated by silica gel column chromatography to give intermediate 92b (21.1 g).

MS(ESI, [M−H]⁺) m/z: 262.9

¹H NMR (500 MHz, DMSO-d₆) δ 10.81 (s, 1H), 8.10-8.04 (m, 1H), 7.73 (dt, J=8.5, 1.0 Hz, 1H), 7.62 (s, 1H), 7.56 (ddd, J=8.4, 6.7, 1.4 Hz, 1H), 7.49 (ddd, J=8.1, 6.8, 1.2 Hz, 1H), 2.61 (s, 3H).

### Step 2: Preparation of Intermediate 92c

Intermediate 92b (21 g), diethyl carbonate (46.4 g), and toluene (200 mL) were added to a reaction flask in sequence. The reaction solution was cooled to 0° C., and sodium hydride (9.43 g) was added in portions. The mixture was heated to 120° C. and reacted. After the reaction was completed, as confirmed by TLC, the reaction solution was cooled to room temperature and slowly poured into 1 L of stirred ice water. The mixture was extracted with 500 mL of ethyl acetate, and the organic phase was discarded. The aqueous phase was adjusted to pH=3 with 3 N hydrochloric acid, the mixture was extracted 3 times with 500 mL of ethyl acetate, and the organic phases were combined. The organic phase was dried over anhydrous sodium sulfate, filtered and concentrated by evaporation at reduced pressure to remove the solvent, thus giving intermediate 92c (20.2 g).

MS(ESI, [M−H]⁺) m/z: 288.9

¹H NMR (500 MHz, DMSO-d₆) δ 8.07 (dd, J=8.4, 1.2 Hz, 1H), 7.85-7.81 (m, 1H), 7.62-7.56 (m, 2H), 7.50 (ddd, J=8.2, 6.9, 1.3 Hz, 1H), 4.08 (s, 2H).

### Step 3: Preparation of Intermediate 92d

Intermediate 92c (20 g), an aqueous hydroxylamine solution (21.05 mL), and ethanol (200 mL) were added to a reaction flask in sequence, and the mixture was reacted at 85° C. After the reaction was completed, as confirmed by TLC, the reaction solution was cooled to room temperature and concentrated by evaporation at reduced pressure to remove the solvent, and 200 mL of ethyl acetate and 200 mL of a saturated aqueous sodium carbonate solution were added to the residue for extraction. The organic phase was separated and the aqueous phase was collected, adjusted to pH of 2-3 with a 3 M aqueous HCl solution, and extracted with 300 mL of ethyl acetate. The organic phase was collected, dried over anhydrous sodium sulfate, filtered, and concentrated by evaporation at reduced pressure to remove the solvent, thus giving intermediate 92d (18.5 g).

MS(ESI, [M−H]⁺) m/z: 303.9

¹H NMR (500 MHz, DMSO-d₆) δ 13.09 (s, 1H), 8.51 (s, 1H), 8.37 (dd, J=8.4, 1.2 Hz, 1H), 8.28-8.21 (m, 1H), 7.87 (ddd, J=8.3, 7.0, 1.3 Hz, 1H), 7.79 (ddd, J=8.3, 7.0, 1.3 Hz, 1H), 4.43 (s, 2H).

### Step 4: Preparation of Intermediate 92e

Intermediate 92d (18 g), ethanol (150 mL), and sulfuric acid (3.13 mL) were added to a reaction flask in sequence, and the mixture was heated to 85° C. and reacted. After the reaction was completed, as confirmed by TLC, the reaction solution was cooled to room temperature and concentrated by evaporation at reduced pressure to remove the solvent. 300 mL of ethyl acetate and 300 mL of water were added to the residue for dilution, and a saturated aqueous sodium bicarbonate solution was added to adjust pH=7. The organic phase was separated, and the aqueous phase was extracted twice with 500 mL of ethyl acetate. The organic phases were combined, dried over anhydrous sodium sulfate, filtered, and concentrated by evaporation at reduced pressure to remove the solvent, thus giving intermediate 92e (17.42 g).

MS(ESI, [M+H]⁺) m/z: 334.12

¹H NMR (500 MHz, DMSO-d₆) δ 8.52 (s, 1H), 8.38 (dd, J=8.4, 1.2 Hz, 1H), 8.19 (dd, J=8.1, 1.0 Hz, 1H), 7.86 (ddd, J=8.2, 7.0, 1.3 Hz, 1H), 7.79 (ddd, J=8.2, 7.0, 1.2 Hz, 1H), 4.54 (s, 2H), 4.15 (q, J=7.1 Hz, 2H), 1.16 (t, J=7.1 Hz, 3H).

### Step 5: Preparation of Intermediate 92f

Intermediate 92e (4 g), azetidin-3-ol (1.050 g), palladium acetate (0.537 g), xantphos (2.77 g), potassium phosphate (7.62 g), and 1,4-dioxane (30 mL) were added to a reaction flask in sequence, and the mixture was heated to 100° C. and reacted under N₂ atmosphere. After the reaction was completed, as confirmed by TLC, the reaction solution was cooled to room temperature, and 100 mL of ethyl acetate and 200 mL of water were added to the system. The organic phase was separated, and the aqueous phase was extracted 3 times with 50 mL of ethyl acetate. The organic phases were combined, dried over anhydrous sodium sulfate, and filtered, and the filtrate was concentrated by evaporation at reduced pressure to remove the solvent. The crude product was separated by silica gel column chromatography to give intermediate 92f (3.03 g).

MS(ESI, [M+H]⁺) m/z: 327.28

¹H NMR (500 MHz, DMSO-d₆) δ 8.07 (dd, J=44.6, 8.4 Hz, 2H), 7.59 (dt, J=77.7, 7.6 Hz, 2H), 6.78 (s, 1H), 5.74 (d, J=6.4 Hz, 1H), 4.64 (h, J=5.9 Hz, 1H), 4.52 (t, J=7.3 Hz, 2H), 4.14 (q, J=7.1 Hz, 2H), 3.93 (dd, J=8.2, 4.7 Hz, 2H), 1.17 (t, J=7.0 Hz, 3H).

### Step 6: Preparation of Intermediate 92g

Intermediate 92f (580 mg), acrylamide (250 mg), and tetrahydrofuran (10 mL) were added to a reaction flask in sequence. The mixture was purged 3 times with nitrogen, and potassium tert-butoxide (299 mg) was added at −15° C. The mixture was reacted at room temperature. After the reaction was completed, as confirmed by TLC, the reaction solution was added dropwise to a saturated aqueous ammonium chloride solution to quench the reaction, and 50 mL of ethyl acetate was added for extraction. The organic phase was separated, and the aqueous phase was extracted 3 times with 50 mL of ethyl acetate. The organic phases were combined, dried over anhydrous sodium sulfate, filtered, and concentrated by evaporation at reduced pressure to remove the solvent. The residue was separated by silica gel column chromatography to give intermediate 92g (0.41 g).

MS(ESI, [M−H]⁺) m/z: 350.12

¹H NMR (500 MHz, DMSO-d₆) δ 8.11 (t, J=7.6 Hz, 2H), 7.66 (ddd, J=8.2, 6.9, 1.2 Hz, 1H), 7.53 (ddd, J=8.3, 6.9, 1.2 Hz, 1H), 6.80 (s, 1H), 5.74 (d, J=6.4 Hz, 1H), 4.91 (dd, J=10.9, 4.9 Hz, 1H), 4.64 (ddd, J=11.4, 6.5, 4.9 Hz, 1H), 4.52 (dt, J=9.8, 7.5 Hz, 2H), 3.92 (dt, J=8.0, 5.2 Hz, 2H), 2.81 (ddd, J=16.9, 11.2, 5.3 Hz, 1H), 2.68-2.58 (m, 2H), 2.41-2.27 (m, 2H).

### Step 7: Preparation of Intermediate 92h

Intermediate 92g (410 mg), acetonitrile (10 mL), and IBX (980 mg) were added to a reaction flask in sequence, and the mixture was heated to 85° C. and reacted under N₂ atmosphere. After the reaction was completed, as confirmed by TLC, the reaction solution was cooled to room temperature and filtered, and the filtrate was concentrated by evaporation at reduced pressure to remove the solvent. The crude product was separated by silica gel column chromatography to give intermediate 92h (0.39 g).

MS(ESI, [M−H]⁺) m/z: 348.21

¹H NMR (500 MHz, DMSO-d₆) δ 11.11 (s, 1H), 8.22-8.14 (m, 2H), 7.70 (ddd, J=8.2, 6.9, 1.2 Hz, 1H), 7.57 (ddd, J=8.4, 7.0, 1.2 Hz, 1H), 7.14 (s, 1H), 5.21-5.08 (m, 4H), 4.96 (dd, J=11.1, 4.8 Hz, 1H), 2.82 (ddd, J=17.0, 11.5, 5.3 Hz, 1H), 2.61 (dt, J=17.2, 4.4 Hz, 1H), 2.57-2.52 (m, 1H), 2.36 (dt, J=13.1, 4.7 Hz, 1H).

### Step 8: Preparation of Compound 92

Intermediate 92h (100 mg), intermediate 69d (110 mg), and 1,2-dichloroethane (10 mL) were added to a reaction flask in sequence, acetic acid (15.78 mg) was added dropwise, and the mixture was stirred at room temperature for 20 min. Sodium cyanoborohydride (49.5 mg) was added, and the mixture was reacted at room temperature. After the reaction was completed, as confirmed by TLC, 20 mL of dichloromethane and 50 mL of water were added to the system for extraction. The organic phase was separated, and the aqueous phase was extracted 3 times with dichloromethane/MeOH=10:1. The organic phases were combined, dried over anhydrous sodium sulfate, filtered, concentrated by evaporation at reduced pressure to remove the solvent, and separated and purified by silica gel column chromatography to give compound 92 (40 mg).

Q-TOF (ESI, [M+H]⁺) m/z: 714.3511

¹H NMR (500 MHz, DMSO-d₆) δ 11.29 (d, J=5.0 Hz, 1H), 11.10 (s, 1H), 8.14 (dd, J=22.1, 8.3 Hz, 2H), 7.73 (s, 1H), 7.71-7.60 (m, 2H), 7.52 (dd, J=8.6, 5.0 Hz, 3H), 7.31 (s, 1H), 7.19 (d, J=8.2 Hz, 2H), 6.82 (d, J=5.4 Hz, 1H), 4.92 (dd, J=10.9, 5.0 Hz, 1H), 4.41 (q, J=8.0 Hz, 2H), 4.05 (q, J=6.5 Hz, 2H), 3.76-3.62 (m, 4H), 3.34 (d, J=3.3 Hz, 2H), 2.99 (d, J=10.4 Hz, 2H), 2.81 (ddd, J=16.9, 11.1, 5.1 Hz, 1H), 2.60 (dt, J=17.1, 4.1 Hz, 1H), 2.47 (s, 1H), 2.42-2.29 (m, 1H), 1.99 (t, J=11.1 Hz, 2H), 1.79 (d, J=12.4 Hz, 2H), 1.62 (dp, J=26.5, 5.3, 4.6 Hz, 8H).

### Example 93: Synthesis of Compound 93

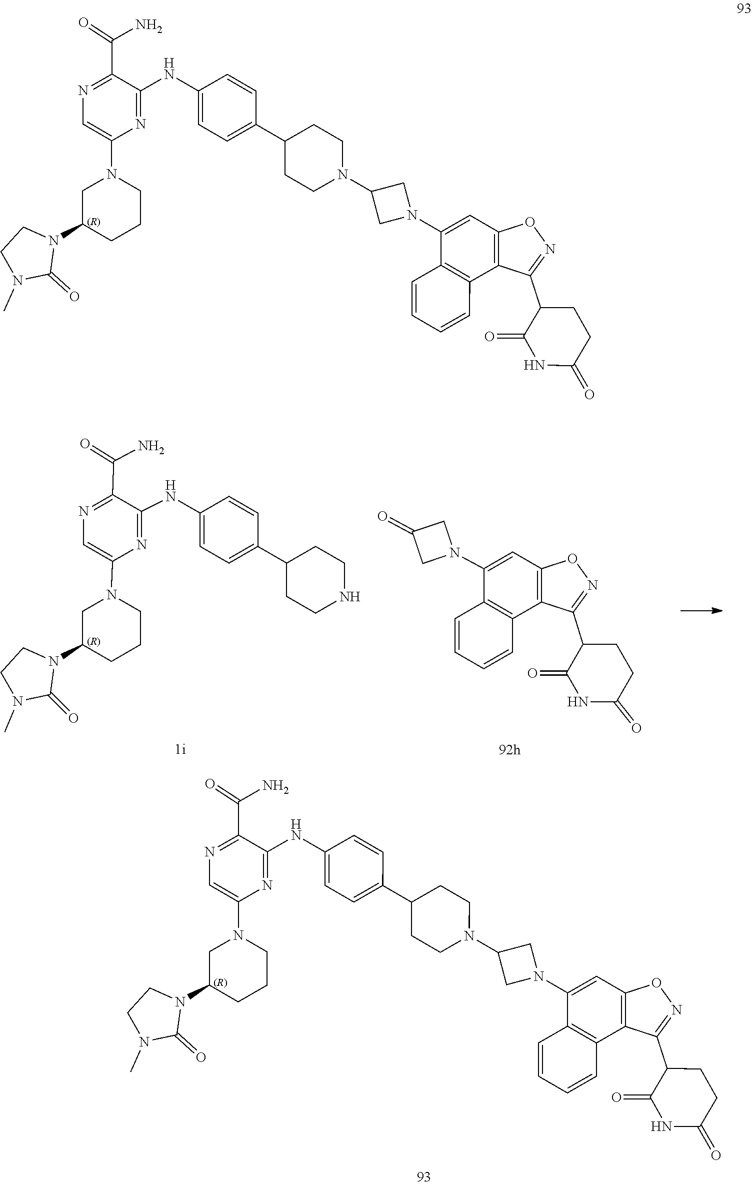

### Step 1: Preparation of Compound 93

Intermediate 92h (100 mg), intermediate 1l (88 mg), 1,2-dichloroethane (10 mL), and isopropanol (1.000 mL) were added to a reaction flask in sequence, acetic acid (12.55 mg) was added dropwise, and the mixture was stirred at room temperature for 20 min. Sodium cyanoborohydride (39.4 mg) was added, and the mixture was reacted at room temperature. After the reaction was completed, as confirmed by TLC, 20 mL of dichloromethane and 50 mL of water were added to the system for extraction. The organic phase was separated, and the aqueous phase was extracted 3 times with dichloromethane/MeOH=10:1. The organic phases were combined, dried over anhydrous sodium sulfate, filtered, concentrated by evaporation at reduced pressure to remove the solvent, and separated and purified by silica gel column chromatography to give compound 93 (40 mg).

Q-TOF (ESI, [M+H]⁺) m/z: 812.3996

¹H NMR (500 MHz, DMSO-d₆) δ 11.20 (s, 1H), 11.10 (s, 1H), 8.14 (dd, J=21.3, 8.0 Hz, 2H), 7.75 (d, J=2.7 Hz, 1H), 7.66 (d, J=5.6 Hz, 2H), 7.51 (dd, J=15.8, 8.1 Hz, 3H), 7.33 (d, J=2.7 Hz, 1H), 7.16 (d, J=8.3 Hz, 2H), 6.81 (s, 1H), 4.92 (dd, J=10.8, 5.0 Hz, 1H), 4.41 (q, J=8.0 Hz, 2H), 4.31 (dd, J=30.8, 12.8 Hz, 2H), 4.10-4.00 (m, 2H), 3.37-3.33 (m, 2H), 3.32-3.29 (m, 2H), 3.28-3.19 (m, 2H), 3.10-2.90 (m, 4H), 2.81 (ddd, J=16.9, 11.2, 5.5 Hz, 1H), 2.69 (s, 3H), 2.60 (dt, J=17.3, 4.5 Hz, 1H), 2.55-2.52 (m, 1H), 2.49-2.45 (m, 1H), 2.34 (dd, J=11.9, 7.4 Hz, 1H), 2.06-1.94 (m, 2H), 1.80 (d, J=11.4 Hz, 5H), 1.70-1.50 (m, 3H).

### Example 94: Synthesis of Compound 94

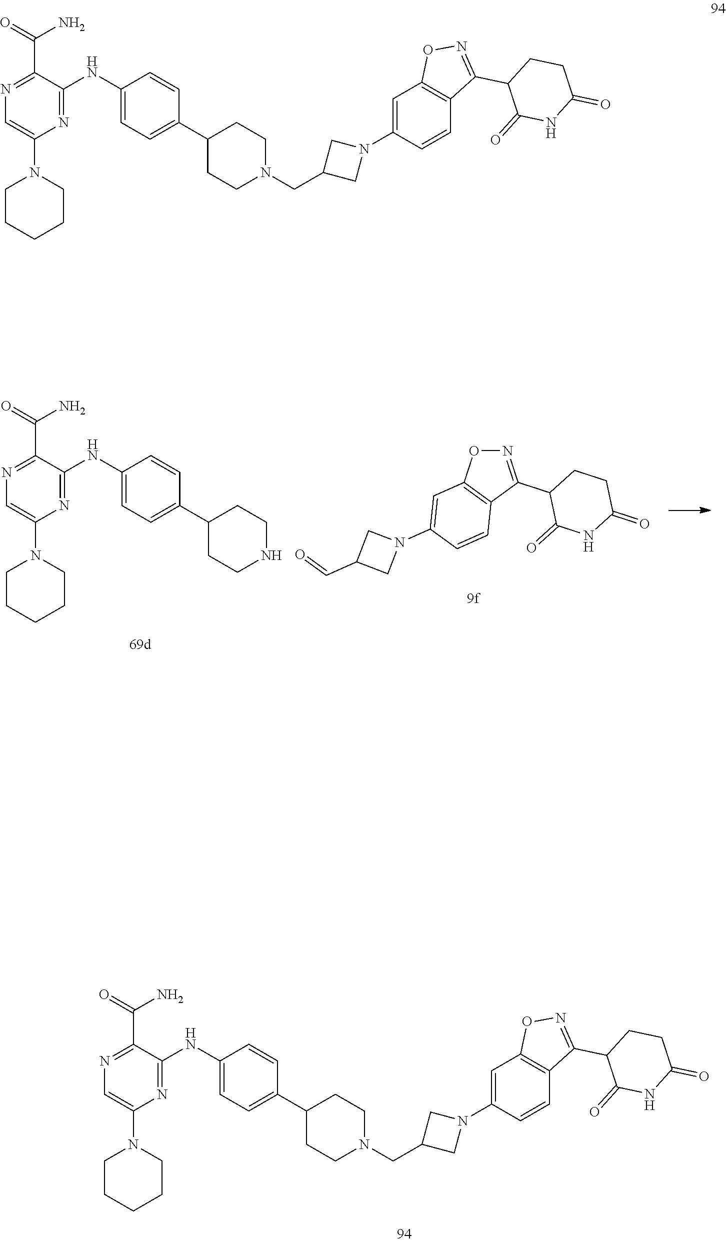

### Step 1: Preparation of Compound 94

Intermediate 69d (80 mg), intermediate 9f (99 mg), dichloromethane (5 mL), and methanol (1 mL) were first added to the reaction solution, and then glacial acetic acid (12.6 mg, 12 IL) was added. After the mixture was stirred for 10 min, sodium cyanoborohydride (26.4 mg) was added. The mixture was stirred overnight at room temperature. A small amount of a NaHCO₃ solution was added to the reaction solution to quench the reaction, and then the mixture was purified by silica gel column chromatography to give compound 94 (80 mg).

MS(ESI, [M+H]⁺) m/z: 677.96

¹H NMR (500 MHz, DMSO-d₆) δ 11.30 (s, 1H), 11.04 (s, 1H), 7.74 (d, J=2.9 Hz, 1H), 7.66 (s, 1H), 7.54 (t, J=9.3 Hz, 3H), 7.32 (d, J=3.0 Hz, 1H), 7.19 (d, J=8.1 Hz, 2H), 6.54-6.46 (m, 2H), 4.44 (dd, J=11.4, 5.0 Hz, 1H), 4.06 (t, J=7.8 Hz, 2H), 3.72-3.59 (m, 6H), 3.34 (s, 3H), 2.74 (ddd, J=17.0, 11.5, 5.3 Hz, 2H), 2.63-2.52 (m, 2H), 2.42 (qd, J=12.0, 4.6 Hz, 2H), 2.17 (dq, J=14.1, 5.0 Hz, 2H), 1.78 (s, 3H), 1.62 (dq, J=32.7, 5.6, 5.2 Hz, 8H).

### Example 95: Synthesis of Compound 95

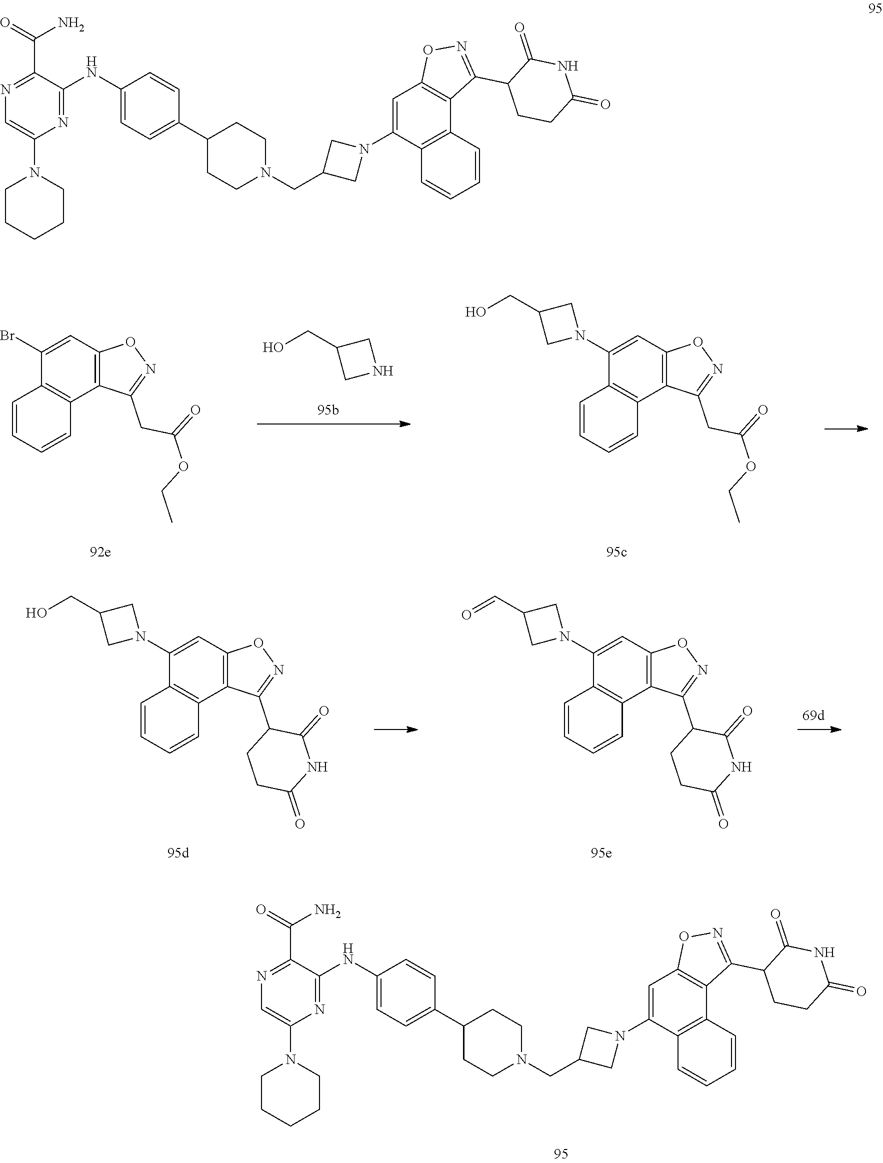

### Step 1: Preparation of Intermediate 95c

Intermediate 92e (3.0 g), intermediate 95b (1.173 g), potassium phosphate (5.72 g), palladium acetate (0.302 g), xantphos (1.558 g), and 1,4-dioxane (50 mL) were added to a reaction flask, and the mixture was heated at 100° C. under nitrogen atmosphere. After the reaction was completed, 10 mL of water was added to the reaction solution, and 30 mL of ethyl acetate was added for extraction. The organic phases were combined and washed with saturated brine, and the extract was dried over anhydrous sodium sulfate, filtered, subjected to rotary evaporation, mixed with silica gel, and purified by column chromatography to give intermediate 95c (3.0 g).

MS(ESI, [M+H]⁺) m/z: 341.30

¹H NMR (500 MHz, DMSO-d₆) δ 8.13 (dd, J=8.6, 1.2 Hz, 1H), 8.01 (dd, J=8.3, 1.2 Hz, 1H), 7.66 (ddd, J=8.2, 5.7, 1.2 Hz, 1H), 7.50 (ddd, J=8.4, 6.9, 1.3 Hz, 1H), 7.35-7.25 (m, 1H), 6.70 (s, 1H), 4.83 (t, J=5.3 Hz, 1H), 4.31 (t, J=8.0 Hz, 4H), 4.14 (q, J=7.1 Hz, 2H), 4.01 (dd, J=7.8, 5.5 Hz, 2H), 3.68-3.60 (m, 1H), 2.85 (tt, J=8.1, 5.7 Hz, 1H), 1.17 (t, J=7.1 Hz, 3H).

### Step 2: Preparation of Intermediate 95d

Intermediate 95c (1.5 g) and acrylamide (0.5 g) were added to a reaction flask. The reaction apparatus was sealed, then anhydrous tetrahydrofuran (30 mL) was added, and a solution of potassium tert-butoxide in THF was slowly added dropwise (the internal temperature was controlled at 0° C.). After the dropwise addition was completed, the temperature was maintained at 0° C. and the mixture was reacted for 30 min, and then the mixture was reacted at room temperature. After the reaction was completed, as confirmed by TLC, the reaction solution was added dropwise to a mixed system of a saturated ammonium chloride solution-ethyl acetate under stirring. The organic phase was separated, dried over anhydrous sodium sulfate, filtered, mixed with silica gel, and purified by column chromatography to give intermediate 95d (581 mg).

MS(ESI, [M+H]⁺) m/z: 366.30

¹H NMR (500 MHz, DMSO-d₆) δ 11.09 (s, 1H), 8.16-8.11 (m, 1H), 7.65 (ddd, J=8.2, 6.9, 1.2 Hz, 1H), 7.54-7.47 (m, 2H), 6.73 (s, 1H), 4.90 (dd, J=10.9, 4.9 Hz, 1H), 4.82 (t, J=5.4 Hz, 1H), 4.31 (q, J=7.9 Hz, 2H), 4.01 (td, J=7.9, 5.4 Hz, 2H), 3.65 (t, J=5.8 Hz, 2H), 2.89-2.75 (m, 2H), 2.59 (dt, J=17.3, 4.6 Hz, 1H), 2.46 (dd, J=10.9, 4.3 Hz, 1H), 2.33 (dq, J=13.8, 5.0 Hz, 1H).

### Step 3: Preparation of Intermediate 95e

Intermediate 95d (280 mg), Dess-Martin periodinane (584 mg), and dichloromethane (20 mL) were added to a reaction flask, and the mixture was stirred at room temperature. After the reaction was completed, the reaction solution was filtered under vacuum, and the filter cake was washed with dichloromethane. The filtrate and the washing solution were combined and subjected to rotary evaporation to dryness, thus giving intermediate 95e (300 mg).

MS(ESI, [M+H]⁺) m/z: 364.2

### Step 4: Preparation of Compound 95

69d (100 mg), intermediate 95e (150 mg), 1,2-dichloroethane (10 mL), and isopropanol (2 mL) were first added to the reaction solution, and then glacial acetic acid (14.2 mg, 14 IL) was added. After the mixture was stirred for 10 min, sodium cyanoborohydride (29.7 mg) was added. The mixture was stirred at room temperature. After the reaction was completed, a small amount of a NaHCO₃ solution was added to the reaction solution to quench the reaction, and then the mixture was purified by silica gel column chromatography to give compound 95 (32 mg).

MS(ESI, [M+H]⁺) m/z: 728.40

¹H NMR (500 MHz, DMSO-d₆) δ 11.29 (s, 1H), 11.10 (s, 1H), 8.12 (dd, J=18.8, 8.4 Hz, 2H), 7.74 (s, 1H), 7.66 (q, J=7.1, 5.8 Hz, 2H), 7.52 (d, J=8.1 Hz, 3H), 7.32 (s, 1H), 7.19 (d, J=8.2 Hz, 2H), 6.76 (s, 1H), 4.91 (dd, J=10.9, 4.9 Hz, 1H), 4.42 (q, J=8.6 Hz, 2H), 3.93 (q, J=6.7 Hz, 2H), 3.68 (t, J=5.4 Hz, 4H), 3.00 (t, J=12.8 Hz, 3H), 2.81 (ddd, J=17.1, 11.0, 5.2 Hz, 2H), 2.59 (dt, J=17.4, 4.6 Hz, 2H), 2.47-2.43 (m, 1H), 2.33 (q, J=6.4, 5.8 Hz, 1H), 2.09 (s, 2H), 1.76 (d, J=11.0 Hz, 2H), 1.66 (q, J=8.0 Hz, 4H), 1.61-1.56 (m, 4H).

### Example 96: Synthesis of Compound 96

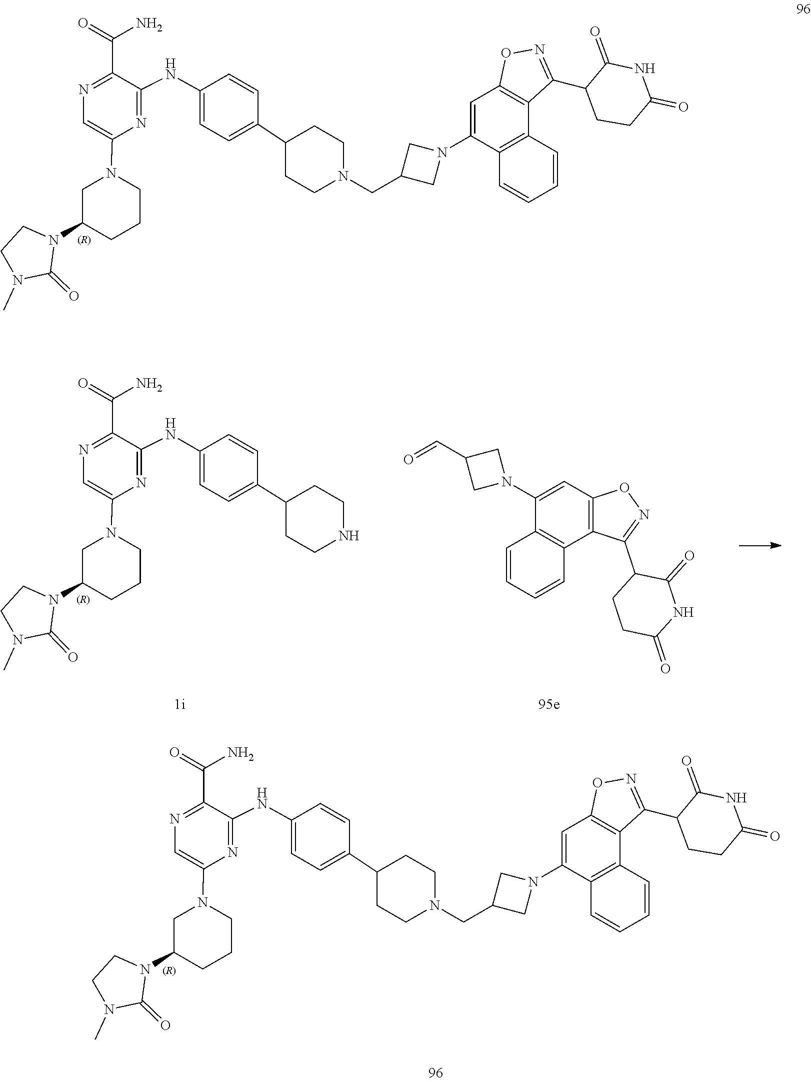

### Step 1: Preparation of Compound 96

Intermediate 1l (120 mg), intermediate 95e (150 mg), 1,2-dichloroethane (10 mL), and isopropanol (2 mL) were first added to the reaction solution, and then glacial acetic acid (13.5 mg, 13 IL) was added. After the mixture was stirred for 10 min, sodium cyanoborohydride (28.4 mg) was added. The mixture was stirred at room temperature. After the reaction was completed, a small amount of a NaHCO₃ solution was added to the reaction solution to quench the reaction, and then the mixture was purified by silica gel column chromatography to give compound % (65 mg).

MS(ESI, [M+H]⁺) m/z: 826.52

¹H NMR (500 MHz, DMSO-d₆) δ 11.20 (s, 1H), 11.10 (s, 1H), 8.12 (dd, J=19.4, 8.4 Hz, 2H), 7.76 (d, J=2.9 Hz, 1H), 7.65 (d, J=9.4 Hz, 2H), 7.51 (dd, J=11.1, 8.1 Hz, 3H), 7.33 (d, J=2.9 Hz, 1H), 7.17 (d, J=8.3 Hz, 2H), 6.75 (s, 1H), 4.91 (dd, J=10.8, 5.0 Hz, 1H), 4.46-4.32 (m, 3H), 4.29 (d, J=13.3 Hz, 1H), 3.92 (q, J=6.6 Hz, 2H), 3.62 (ddt, J=11.0, 8.7, 4.0 Hz, 1H), 3.31-3.22 (m, 3H), 3.08-2.91 (m, 4H), 2.81 (ddd, J=16.8, 11.2, 5.3 Hz, 1H), 2.72 (s, 3H), 2.66 (d, J=7.3 Hz, 2H), 2.63-2.51 (m, 2H), 2.45 (ddt, J=24.5, 12.0, 2.9 Hz, 3H), 2.37-2.29 (m, 1H), 2.07 (td, J=11.6, 2.41 Hz, 2H), 1.86-1.71 (m, 5H), 1.68-1.52 (m, 3H).

### Example 97: Synthesis of Compound 97

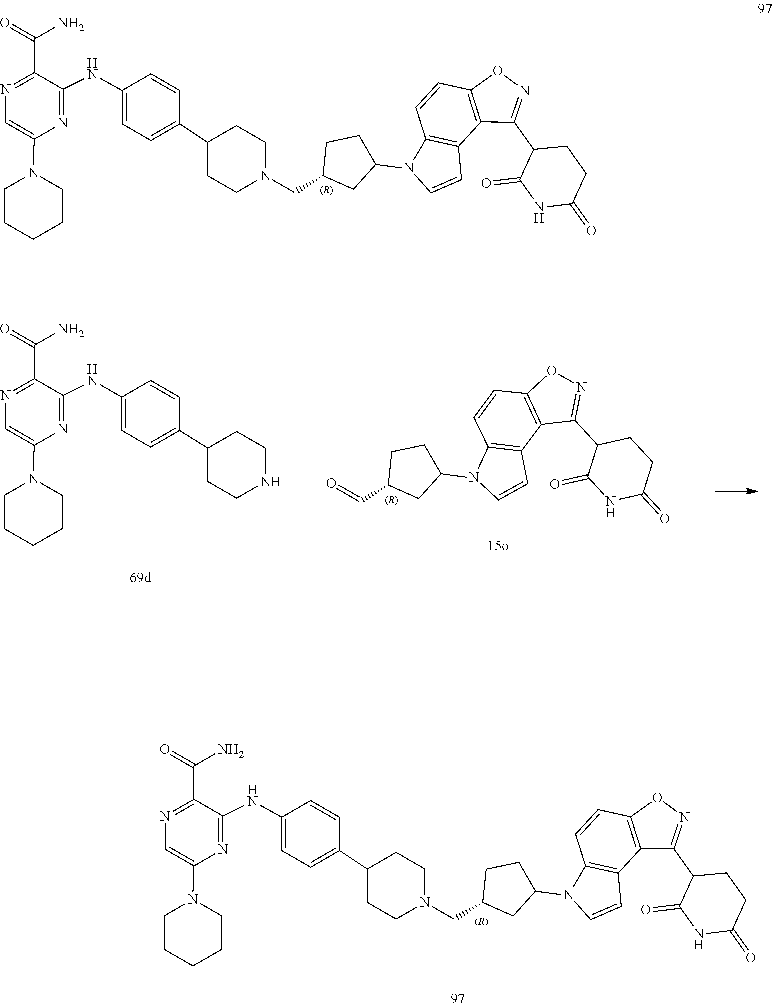

### Step 1: Preparation of Compound 97

Intermediate 69d (100 mg), intermediate 15o (144 mg), dichloromethane (10 mL), and MeOH (1.000 mL) were added to a reaction flask in sequence, acetic acid (15.78 mg) was added dropwise, and the mixture was stirred at room temperature for 20 min. Sodium cyanoborohydride (49.5 mg) was added, and the mixture was reacted at room temperature. After the reaction was completed, as confirmed by TLC, 20 mL of dichloromethane and 50 mL of water were added to the system for extraction. The organic phase was separated, and the aqueous phase was extracted 3 times with dichloromethane/MeOH=10:1. The organic phases were combined, dried over anhydrous sodium sulfate, filtered, and concentrated by evaporation at reduced pressure to remove the solvent, and the crude product was separated by silica gel column chromatography to give compound 97 (40 mg).

Q-TOF (ESI, [M+H]⁺) m/z: 730.3822

¹H NMR (500 MHz, DMSO-d₆) δ 11.28 (s, 1H), 11.18 (s, 1H), 7.91 (dd, J=9.2, 2.2 Hz, 1H), 7.78-7.69 (m, 2H), 7.66 (s, 1H), 7.50 (dd, J=10.9, 8.6 Hz, 3H), 7.31 (d, J=2.9 Hz, 1H), 7.17 (d, J=8.3 Hz, 2H), 6.58 (dd, J=7.8, 3.1 Hz, 1H), 5.04 (p, J=8.0 Hz, 1H), 4.69 (dd, J=11.8, 5.1 Hz, 1H), 3.67 (t, J=5.3 Hz, 4H), 3.01 (s, 2H), 2.85 (ddd, J=17.4, 12.1, 5.4 Hz, 1H), 2.63 (dt, J=17.2, 4.2 Hz, 1H), 2.48-2.39 (m, 3H), 2.38 (s, 3H), 2.23 (ddt, J=15.5, 11.3, 6.7 Hz, 2H), 2.09-1.87 (m, 5H), 1.74 (d, J=11.5 Hz, 2H), 1.69-1.54 (m, 9H).

### Example 98: Synthesis of Compounds 98-1 and 98-2

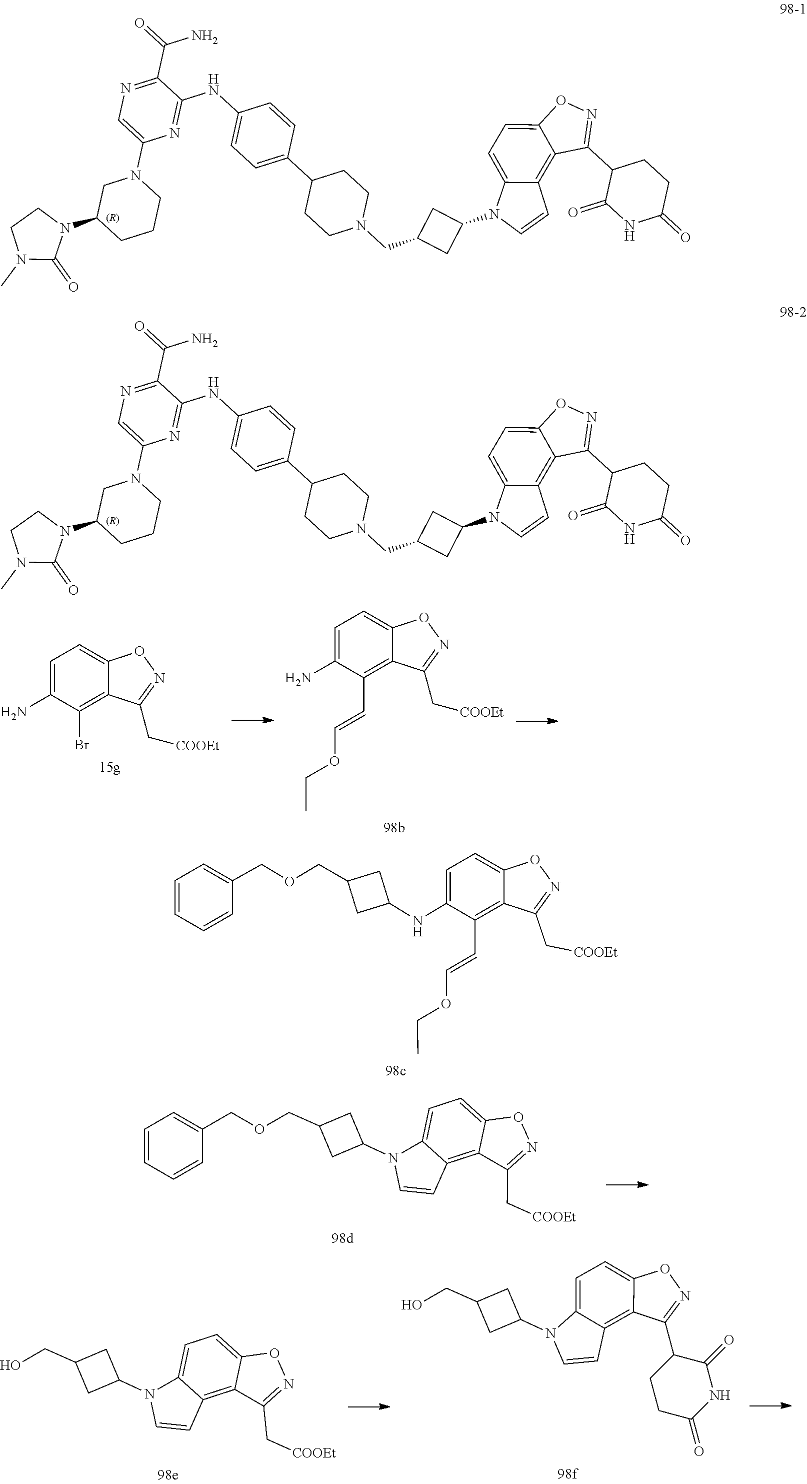

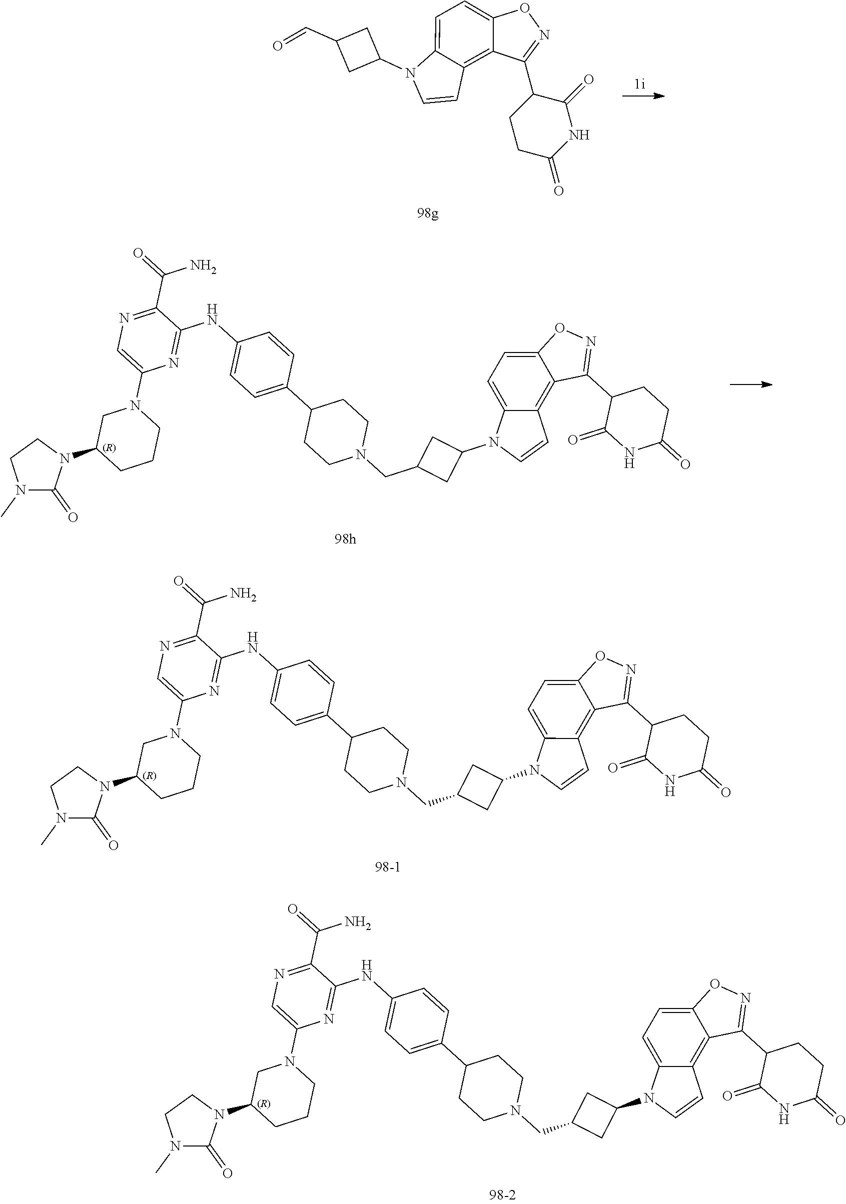

### Step 1: Preparation of Intermediate 98b

Intermediate 15g (30.0 g), ethoxyvinyl borate (24.83 g), cesium carbonate (94.25 g), palladium acetate (2.16 g), Ruphos (9.00 g), and 1,4-dioxane (500 mL) were added to a reaction flask, and the mixture was stirred at 100° C. After the reaction was completed, as confirmed by TLC, 1000 mL of water was added to the reaction solution, and the mixture was extracted twice with 500 mL of ethyl acetate. The organic phases were combined, dried over anhydrous sodium sulfate, filtered under vacuum and concentrated, and the residue was purified by silica gel column chromatography to give intermediate 98b (14.50 g).

MS(ESI, [M+H]+) m/z: 291.11.

¹H NMR (500 MHz, DMSO-d₆) δ 7.29 (d, J=8.8 Hz, 1H), 7.01 (d, J=8.8 Hz, 1H), 6.70 (d, J=13.1 Hz, 1H), 5.64 (d, J=13.1 Hz, 1H), 4.89 (s, 2H), 4.11-4.04 (m, 2H), 3.95-3.89 (m, 2H), 1.29 (t, J=7.0 Hz, 3H), 1.18 (t, J=7.1 Hz, 3H).

### Step 2: Preparation of Intermediate 98c

Intermediate 98b (5.0 g), 3-(benzyloxymethyl)-1-cyclobutanone (4.42 g), methanol (50 mL), and glacial acetic acid (0.465 g, 7.75 mmol) were added to a reaction flask. After the mixture was stirred at room temperature for 5 min, sodium cyanoborohydride (1.606 g, 25.6 mmol) was added, and the mixture was heated at 60° C. After the reaction was completed, the reaction solution was extracted twice with 300 mL of water and 300 mL of dichloromethane. The extracts were combined, dried over anhydrous sodium sulfate, filtered under vacuum, and concentrated, and the residue was purified by silica gel column chromatography to give intermediate 98c (4.73 g).

MS(ESI, [M+H]+) m/z: 465.30.

### Step 3: Preparation of Intermediate 98d

Intermediate 98c (4.60 g), dichloromethane (50 mL), and hydrochloric acid (5.94 mL, 4 mol/L, 23.76 mmol) were added to a reaction flask in sequence, and the mixture was reacted at room temperature. After the reaction was completed, as confirmed by TLC, 200 mL of an aqueous NaHCO₃ solution and 200 mL of dichloromethane were added to the reaction solution. The organic phase was separated, dried over anhydrous sodium sulfate, filtered, and concentrated. The concentrate was separated and purified by silica gel column chromatography to give intermediate 98d (3.56 g).

MS(ESI, [M+H]⁺) m/z: 419.33

### Step 4: Preparation of Intermediate 98e

Intermediate 98d (3.50 g), 10% palladium on carbon catalyst (1.75 g), MeOH (35 mL), and hydrochloric acid (2.01 mL, 4 mol/L) were added to a reaction flask in sequence, and the mixture was reacted at room temperature under H₂ atmosphere. After the reaction was completed, as confirmed by TLC, the reaction solution was filtered under vacuum, and the filtrate was concentrated and separated and purified by silica gel column chromatography to give intermediate 98e (2.05 g).

MS(ESI, [M+H]⁺) m/z: 329.32

### Step 5: Preparation of Intermediate 98f

Intermediate 98e (2.0 g), acrylamide (0.9 g), and anhydrous tetrahydrofuran (30 mL) were added to a reaction flask in sequence. A solution of potassium tert-butoxide in THF (1.03 g, 9.14 mL, 9.14 mmol) was slowly added under an ice bath, and the mixture was reacted for 1 h. The reaction solution was added to 100 mL of a saturated aqueous ammonium chloride solution to quench the reaction, and the mixture was extracted with 100 mL of dichloromethane. The organic phase was separated, dried over anhydrous sodium sulfate, filtered, and concentrated. The concentrate was separated and purified by silica gel column chromatography to give intermediate 98f (1.40 g).

MS(ESI, [M+H]⁺) m/z: 354.35.

¹H NMR (500 MHz, DMSO-d₆) δ 11.18 (s, 1H), 7.89-7.70 (m, 2H), 7.48 (dd, J=9.1, 1.5 Hz, 1H), 6.60 (dd, J=6.2, 3.1 Hz, 1H), 5.27-4.93 (m, 1H), 4.76-4.62 (m, 2H), 3.55 (dt, J=79.3, 5.5 Hz, 2H), 3.30 (d, J=9.6 Hz, 1H), 2.85 (ddd, J=17.4, 12.1, 5.4 Hz, 1H), 2.67-2.51 (m, 3H), 2.47-2.38 (m, 2H), 2.22 (ddd, J=13.6, 10.8, 6.6 Hz, 2H).

### Step 6: Preparation of Intermediate 98g

Intermediate 98f (1.2 g), acetonitrile (10.00 mL), and IBX oxidant (1.43 g) were added to a reaction flask in sequence, and the mixture was reacted at 80° C. After the reaction was completed, as confirmed by TLC, the reaction solution was filtered, and the filtrate was concentrated to give intermediate 98g (1.39 g).

MS(ESI, [M+H]⁺) m/z: 352.36.

### Step 7: Preparation of Compounds 98-1 and 98-2

Intermediate 1l (480 mg), intermediate 98g (500 mg), dichloroethane (10 mL), isopropanol (2 mL), and glacial acetic acid (35.6 mg, 0.034 mL) were added to a reaction flask. After the mixture was stirred at room temperature for 30 min, sodium triacetoxyborohydride (503 mg) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give compound 98h (544 mg). Compound 98h was separated by high performance liquid chromatography to give compound 98-1 (248 mg) and compound 98-2 (165 mg) in sequence. The conditions of preparative chromatography were as follows:

Instrument and preparative column: Shimadzu LC-20AD high performance liquid chromatograph was used, and the preparative column was Ultimate XB-Phenyl (4.6×250 mm, 10 μm). Mobile phase system: acetonitrile/10 mM ammonium acetate, isocratic elution: acetonitrile/10 mM ammonium acetate=60/40.

MS(ESI, [M+H]+) m/z: 814.66.

Compound 98-1: ¹H NMR (500 MHz, DMSO-d₆) δ 11.20 (d, J=19.5 Hz, 2H), 7.84 (d, J=9.1 Hz, 1H), 7.78 (dd, J=12.5, 3.0 Hz, 2H), 7.66 (s, 1H), 7.51 (dd, J=11.1, 8.3 Hz, 3H), 7.36-7.31 (m, 1H), 7.17 (d, J=8.2 Hz, 2H), 6.61 (d, J=3.1 Hz, 1H), 5.05-4.95 (m, 1H), 4.70 (dd, J=11.8, 5.1 Hz, 1H), 4.35 (d, J=12.3 Hz, 1H), 4.32-4.25 (m, 1H), 3.62 (tt, J=10.8, 4.3 Hz, 1H), 3.30-3.20 (m, 4H), 3.04 (t, J=11.7 Hz, 2H), 2.96 (t, J=12.5 Hz, 2H), 2.86 (ddd, J=17.2, 12.1, 5.4 Hz, 2H), 2.76 (s, 2H), 2.71 (s, 3H), 2.63 (dt, J=17.1, 4.2 Hz, 2H), 2.43 (td, J=12.6, 4.6 Hz, 2H), 2.23 (dq, J=13.6, 4.8 Hz, 2H), 2.15 (s, 3H), 1.87-1.65 (m, 7H), 1.56 (d, J=12.1 Hz, 2H).

Compound 98-2: ¹H NMR (500 MHz, DMSO-d₆) δ 11.19 (d, J=4.6 Hz, 2H), 7.89-7.83 (m, 2H), 7.76 (d, J=2.9 Hz, 1H), 7.66 (s, 1H), 7.49 (dd, J=8.7, 5.8 Hz, 3H), 7.33 (d, J=2.9 Hz, 1H), 7.17 (d, J=8.2 Hz, 2H), 6.61 (d, J=3.1 Hz, 1H), 5.30 (p, J=7.6 Hz, 1H), 4.70 (dd, J=11.9, 5.1 Hz, 1H), 4.36 (d, J=12.4 Hz, 1H), 4.28 (d, J=13.2 Hz, 1H), 3.61 (ddt, J=11.1, 8.5, 4.2 Hz, 1H), 3.29 (d, J=7.7 Hz, 2H), 3.25 (dd, J=9.7, 7.2 Hz, 2H), 2.99 (dt, J=37.5, 12.1 Hz, 4H), 2.85 (ddd, J=17.3, 12.2, 5.4 Hz, 1H), 2.71 (s, 3H), 2.66-2.60 (m, 3H), 2.59-2.54 (m, 2H), 2.47-2.35 (m, 4H), 2.23 (dq, J=13.7, 4.9 Hz, 1H), 2.05 (d, J=27.3 Hz, 2H), 1.85-1.72 (m, 5H), 1.70-1.50 (m, 4H).

### Example 99: Synthesis of Compounds 99-1 and 99-2

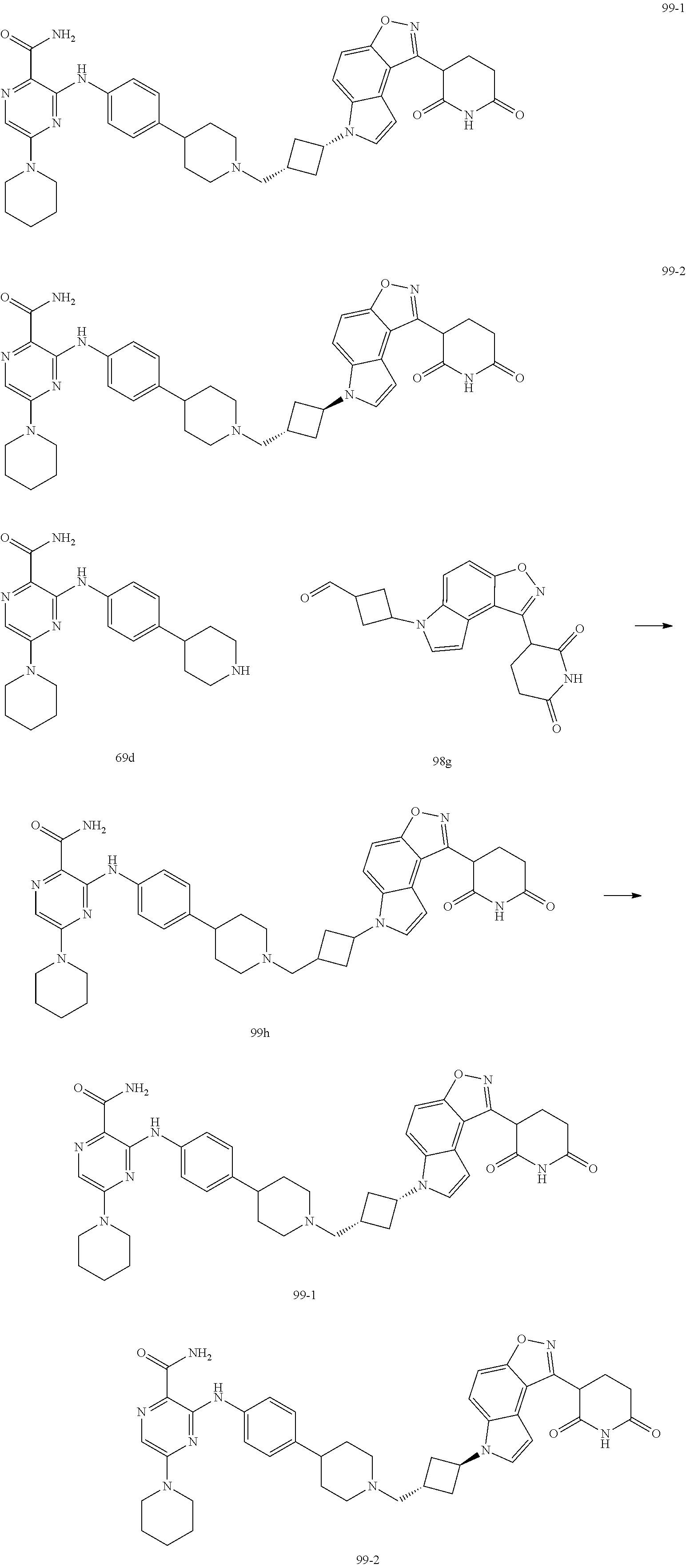

### Step 1: Preparation of Compounds 99-1 and 99-2

Intermediate 69d (200 mg), intermediate 98g (200 mg), dichloroethane (10 mL), isopropanol (2 mL), and glacial acetic acid (14.2 mg, 0.013 mL) were added to a reaction flask. After the mixture was stirred at room temperature (30 min), sodium cyanoborohydride (503 mg) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give compound 99h (200 mg). Compound 99h was separated by high performance liquid chromatography to give compound 99-1 (95 mg) and compound 99-2 (37 mg) in sequence. The conditions of preparative chromatography were as follows:

Instrument and preparative column: Shimadzu LC-20AD high performance liquid chromatograph was used, and the preparative column was Ultimate XB-Phenyl (4.6×250 mm, 10 μm). Mobile phase system: acetonitrile/10 mM ammonium acetate, isocratic elution: acetonitrile/10 mM ammonium acetate=60/40.

MS(ESI, [M+H]⁺) m/z: 716.44

Compound 99-1: ¹H NMR (500 MHz, DMSO-d₆) δ 11.29 (s, 1H), 11.18 (s, 1H), 7.87-7.77 (m, 2H), 7.74 (d, J=2.9 Hz, 1H), 7.66 (s, 1H), 7.51 (dd, J=19.4, 8.5 Hz, 3H), 7.32 (d, J=2.9 Hz, 1H), 7.19 (d, J=8.1 Hz, 2H), 6.60 (d, J=3.1 Hz, 1H), 4.99 (p, J=8.5 Hz, 1H), 4.70 (dd, J=11.9, 5.1 Hz, 1H), 3.67 (t, J=5.3 Hz, 4H), 2.98 (s, 2H), 2.85 (td, J=12.1, 6.0 Hz, 1H), 2.74 (d, J=9.5 Hz, 2H), 2.63 (dt, J=17.1, 4.2 Hz, 1H), 2.43 (tt, J=12.2, 6.2 Hz, 3H), 2.23 (dq, J=13.8, 4.9 Hz, 1H), 2.18-1.97 (m, 4H), 1.76 (s, 2H), 1.66 (q, J=6.0 Hz, 4H), 1.59 (q, J=5.5 Hz, 4H), 1.23 (s, 2H).

Compound 99-2: ¹H NMR (500 MHz, DMSO-d₆) δ 11.29 (s, 1H), 11.19 (s, 1H), 7.85 (d, J=9.5 Hz, 2H), 7.74 (s, 1H), 7.66 (s, 1H), 7.51 (dd, J=18.6, 8.4 Hz, 3H), 7.32 (s, 1H), 7.19 (d, J=8.1 Hz, 2H), 6.61 (d, J=3.3 Hz, 1H), 5.29 (p, J=7.5 Hz, 1H), 4.70 (dd, J=11.7, 5.1 Hz, 1H), 3.68 (d, J=5.5 Hz, 4H), 3.04 (s, 2H), 2.86 (ddd, J=17.1, 11.7, 5.3 Hz, 1H), 2.73-2.60 (m, 6H), 2.42 (dt, J=19.5, 11.1 Hz, 3H), 2.28-2.20 (m, 1H), 2.04 (d, J=52.3 Hz, 2H), 1.78 (d, J=11.3 Hz, 2H), 1.72-1.63 (m, 4H), 1.59 (s, 4H).

### Example 100: Synthesis of Compound 100

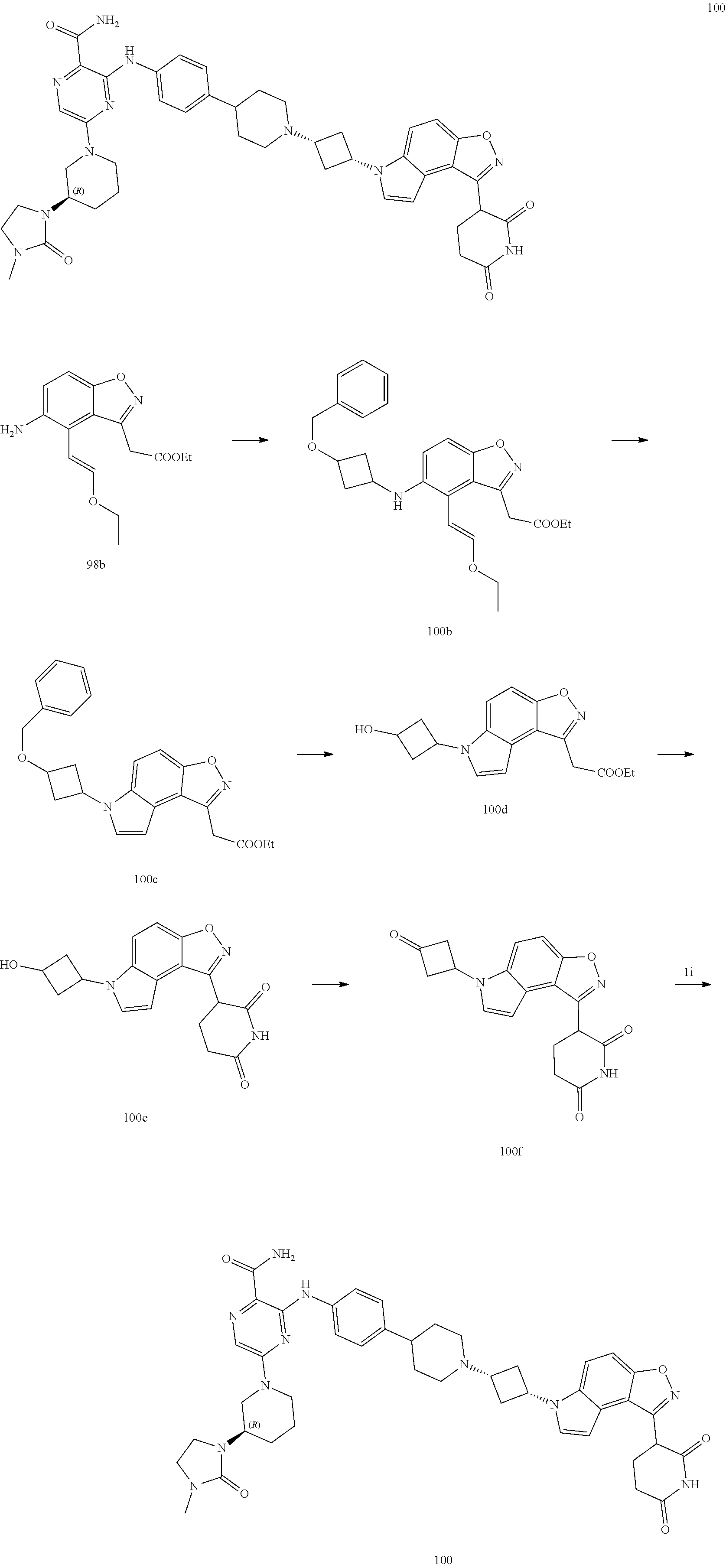

### Step 1: Preparation of Intermediate 100b

Intermediate 98b (5.3 g), 3-(benzyloxy)-1-cyclobutanone (3.38 g), methanol (50 mL), and glacial acetic acid (0.384 g) were added to a reaction flask. After the mixture was stirred at room temperature for 5 min, sodium cyanoborohydride (1.606 g) was added, and the mixture was stirred overnight at room temperature. After the reaction was completed, the reaction solution was extracted twice with 300 mL of water and 300 mL of dichloromethane. The extracts were combined, dried over anhydrous sodium sulfate, filtered under vacuum, and concentrated, and the residue was purified by silica gel column chromatography to give intermediate 100b (3.95 g).

MS(ESI, [M+H]+) m/z: 451.38.

¹H NMR (500 MHz, DMSO-d₆) δ 7.41-7.37 (m, 1H), 7.35-7.32 (m, 4H), 7.30-7.28 (m, 1H), 6.91-6.76 (m, 1H), 6.67 (t, J=13.3 Hz, 1H), 5.65 (dd, J=13.0, 7.8 Hz, 1H), 4.92 (dd, J=10.5, 6.5 Hz, 1H), 4.42-4.37 (m, 2H), 4.21-4.11 (m, 2H), 4.10-4.04 (m, 4H), 3.94 (q, J=7.0 Hz, 2H), 2.73 (dtdd, J=9.2, 6.7, 4.8, 2.9 Hz, 2H), 2.37 (tt, J=10.7, 4.9 Hz, 1H), 1.85 (qd, J=8.5, 2.8 Hz, 1H), 1.30 (td, J=7.0, 1.6 Hz, 3H), 1.20-1.17 (m, 3H).

### Step 2: Preparation of Intermediate 100c

Intermediate 100b (3.95 g), dichloromethane (40 mL), and hydrochloric acid (6.58 mL, 4 mol/L) were added to a reaction flask in sequence, and the mixture was reacted at room temperature. After the reaction was completed, as confirmed by TLC, 200 mL of an aqueous NaHCO₃ solution and 200 mL of dichloromethane were added to the reaction solution. The organic phase was separated, dried over anhydrous sodium sulfate, filtered, and concentrated. The concentrate was separated and purified by silica gel column chromatography to give intermediate 100c (2.51 g).

MS(ESI, [M+H]⁺) m/z: 405.3

¹H NMR (500 MHz, DMSO-d₆) δ 7.87-7.75 (m, 2H), 7.47 (dd, J=9.1, 7.2 Hz, 1H), 7.41-7.27 (m, 6H), 6.69 (t, J=3.6 Hz, 1H), 4.48 (s, 2H), 4.42-4.33 (m, 1H), 4.26 (d, J=1.4 Hz, 2H), 4.14 (qd, J=7.1, 2.3 Hz, 2H), 4.05-3.96 (m, 1H), 2.97 (dtd, J=9.1, 6.7, 2.9 Hz, 1H), 2.69 (dt, J=8.5, 5.4 Hz, 1H), 2.34 (ddd, J=11.7, 9.1, 7.4 Hz, 1H), 1.18 (td, J=7.1, 3.0 Hz, 3H).

### Step 3: Preparation of Intermediate 100d

Intermediate 100c (1.5 g), 10% palladium on carbon catalyst (0.75 g), MeOH (30 mL), and hydrochloric acid (0.926 mL, 4 mol/L) were added to a reaction flask in sequence, and the mixture was reacted at room temperature under H₂ atmosphere. After the reaction was completed, as confirmed by TLC, the reaction solution was filtered under vacuum, and the filtrate was concentrated and separated and purified by silica gel column chromatography to give intermediate 100d (0.765 g).

MS(ESI, [M+H]⁺) m/z: 315.2

¹H NMR (500 MHz, DMSO-d₆) δ 7.83-7.77 (m, 1H), 7.77-7.71 (m, 1H), 7.46 (dd, J=9.1, 4.1 Hz, 1H), 6.67 (t, J=2.8 Hz, 1H), 5.25 (tt, J=8.2, 5.8 Hz, 1H), 4.61 (tt, J=9.3, 7.2 Hz, 1H), 4.31-4.24 (m, 2H), 4.12 (dq, J=13.5, 7.1 Hz, 2H), 4.07-4.00 (m, 1H), 2.93 (dtd, J=9.7, 6.9, 2.9 Hz, 1H), 2.71-2.59 (m, 1H), 2.53 (dd, J=8.2, 4.2 Hz, 1H), 2.29-2.22 (m, 1H), 1.18 (td, J=7.1, 1.5 Hz, 3H).

### Step 4: Preparation of Intermediate 100e

Intermediate 100d (1.7 g), acrylamide (0.461 g), and anhydrous tetrahydrofuran (30 mL) were added to a reaction flask in sequence. Potassium tert-butoxide (0.91 g) was slowly added under an ice bath, and the mixture was reacted for 1 h. The reaction solution was added to 100 mL of a saturated aqueous ammonium chloride solution to quench the reaction, and the mixture was extracted with 100 mL of dichloromethane. The organic phase was separated, dried over anhydrous sodium sulfate, filtered, and concentrated. The concentrate was separated and purified by silica gel column chromatography to give intermediate 100e (0.84 g).

MS(ESI, [M+H]⁺) m/z: 340.2.

### Step 5: Preparation of Intermediate 100f

Intermediate 100e (400 mg), acetonitrile (10.00 mL), and IBX oxidant (353 mg) were added to a reaction flask in sequence, and the mixture was reacted at 80° C. After the reaction was completed, as confirmed by TLC, the reaction solution was directly filtered to remove solids, and the filtrate was concentrated to give intermediate 100f (410 mg).

MS(ESI, [M+H]⁺) m/z: 338.3.

### Step 6: Preparation of Compound 100

Intermediate 1l (200 mg), intermediate 100f (165 mg), dichloroethane (10 mL), isopropanol (2 mL), and glacial acetic acid (11.29 mg, 0.011 mL) were added to a reaction flask. After the mixture was stirred at room temperature for 15 min, sodium cyanoborohydride (47.27 mg) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give 160 mg of a mixture. The mixture was separated by high performance liquid chromatography to give compound 100 (62 mg). The conditions of preparative chromatography were as follows: Instrument and preparative column: Shimadzu LC-20AD high performance liquid chromatograph was used, and the preparative column was Ultimate XB-Phenyl (4.6×250 mm, 10 μm). Mobile phase system: acetonitrile/10 mM ammonium acetate, isocratic elution: acetonitrile/10 mM ammonium acetate=60/40.

MS(ESI, [M+H]+) m/z: 800.43.

¹H NMR (500 MHz, DMSO-d₆) δ 11.20 (d, J=11.3 Hz, 2H), 7.87 (d, J=8.7 Hz, 1H), 7.77 (s, 2H), 7.66 (s, 1H), 7.51 (d, J=8.2 Hz, 3H), 7.34 (s, 1H), 7.17 (d, J=8.0 Hz, 2H), 6.62 (s, 1H), 4.87 (s, 1H), 4.70 (dd, J=11.8, 5.1 Hz, 1H), 4.35 (d, J=12.7 Hz, 1H), 4.29 (d, J=12.7 Hz, 1H), 3.66-3.58 (m, 1H), 3.26 (p, J=7.6, 6.6 Hz, 2H), 3.11-2.94 (m, 4H), 2.93-2.75 (m, 4H), 2.75-2.67 (m, 4H), 2.64 (dd, J=17.3, 3.9 Hz, 1H), 2.44 (dd, J=12.6, 4.6 Hz, 2H), 2.30-2.16 (m, 3H), 2.08 (d, J=1.8 Hz, 1H), 1.92 (s, 2H), 1.79 (d, J=14.8 Hz, 5H), 1.68-1.53 (m, 3H).

### Example 101: Synthesis of Compound 101

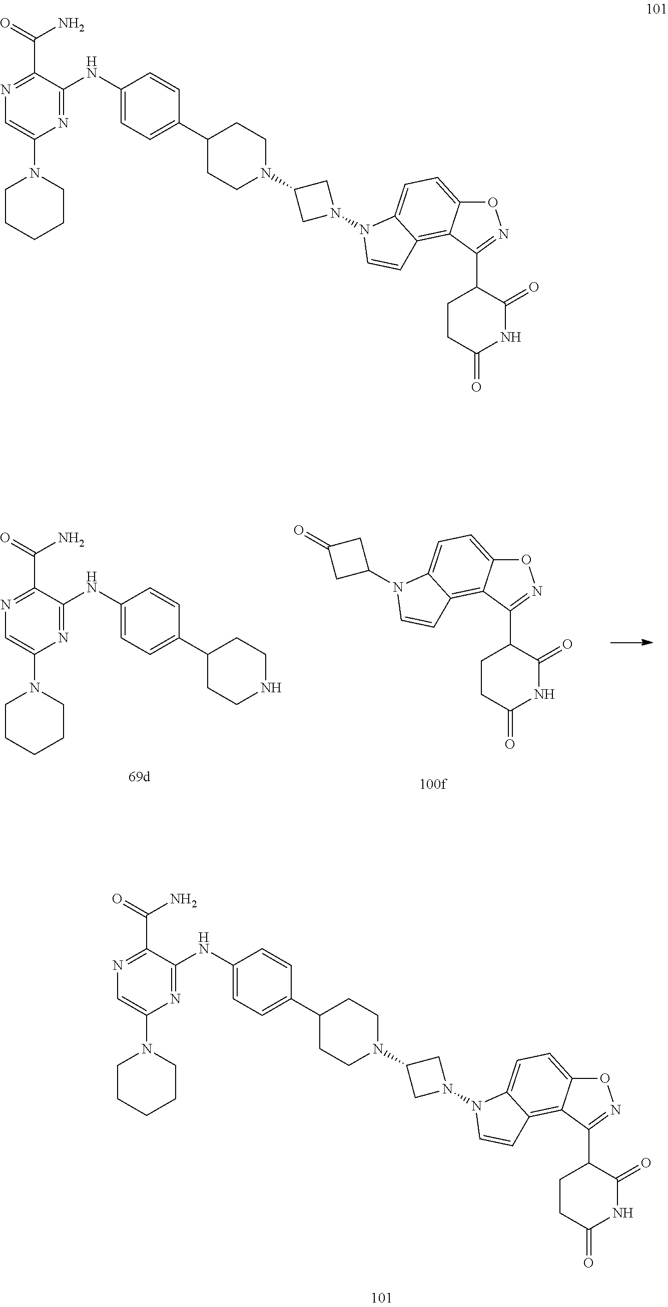

### Step 1: Preparation of Compound 101

Intermediate 69d (150 mg), intermediate 100f (160 mg), dichloroethane (10 mL), isopropanol (2 mL), and glacial acetic acid (11.84 mg, 0.011 mL) were added to a reaction flask. After the mixture was stirred at room temperature (30 min), sodium cyanoborohydride (74.3 mg) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give 120 mg of a mixture. The mixture was separated by high performance liquid chromatography to give compound 101 (49 mg). The conditions of preparative chromatography were as follows: Instrument and preparative column: Shimadzu LC-20AD high performance liquid chromatograph was used, and the preparative column was Ultimate XB-Phenyl (4.6×250 mm, 10 μm). Mobile phase system: acetonitrile/10 mM ammonium acetate, isocratic elution: acetonitrile/10 mM ammonium acetate=60/40.

MS(ESI, [M+H]+) m/z: 702.38.

¹H NMR (500 MHz, DMSO-d₆) δ 11.29 (s, 1H), 11.19 (s, 1H), 7.87 (d, J=9.2 Hz, 1H), 7.75 (dd, J=15.7, 3.0 Hz, 2H), 7.66 (s, 1H), 7.51 (dd, J=10.3, 8.7 Hz, 3H), 7.31 (d, J=2.7 Hz, 1H), 7.19 (d, J=8.3 Hz, 2H), 6.61 (d, J=3.1 Hz, 1H), 4.85 (p, J=8.5 Hz, 1H), 4.70 (dd, J=11.9, 5.1 Hz, 1H), 3.67 (t, J=5.3 Hz, 4H), 2.98 (d, J=10.6 Hz, 2H), 2.86 (td, J=12.0, 6.0 Hz, 1H), 2.79 (q, J=9.8, 8.1 Hz, 2H), 2.65 (ddd, J=17.4, 12.7, 5.7 Hz, 2H), 2.49-2.38 (m, 2H), 2.28-2.16 (m, 3H), 1.91 (t, J=11.2 Hz, 2H), 1.78 (d, J=11.5 Hz, 2H), 1.65 (dd, J=9.5, 4.7 Hz, 3H), 1.59 (h, J=5.0, 4.5 Hz, 5H).

### Example 102: Synthesis of Compounds 102-1 and 102-2

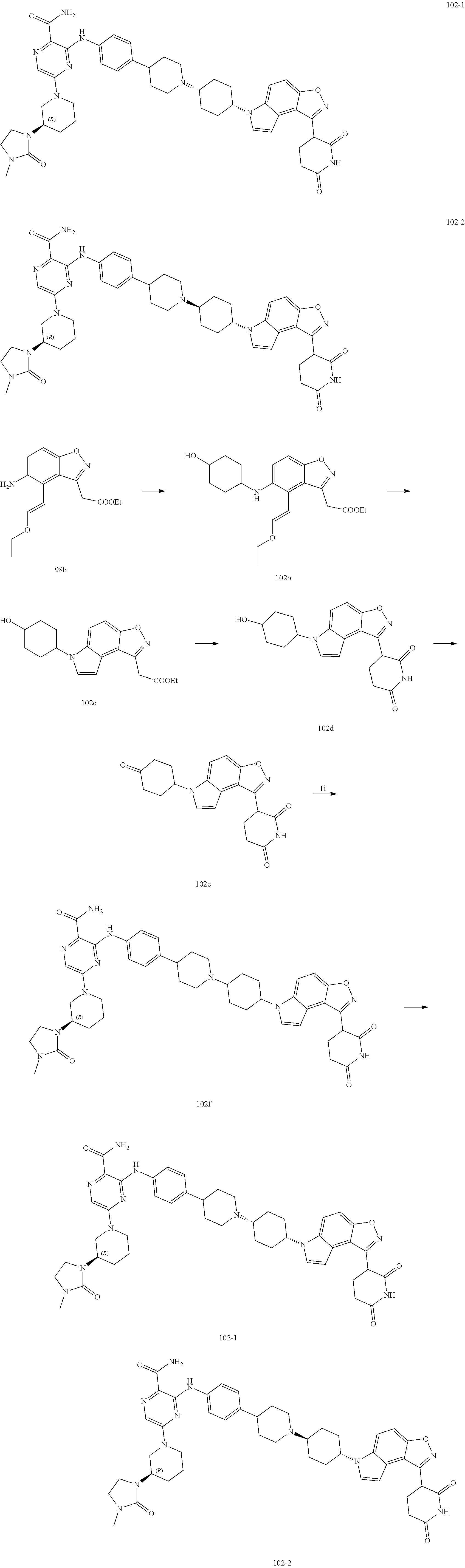

### Step 1: Preparation of Intermediate 102b

Intermediate 98b (1.0 g), 4-hydroxycyclohexanone (0.662 g), methanol (20 mL), and glacial acetic acid (0.384 g) were added to a reaction flask. After the mixture was stirred at room temperature for 5 min, sodium cyanoborohydride (1.606 g) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give intermediate 102b (0.71 g).

MS(ESI, [M+H]⁺) m/z: 389.31.

¹H NMR (500 MHz, DMSO-d₆) δ 7.44-7.38 (m, 1H), 7.05 (d, J=9.1 Hz, 1H), 6.66 (d, J=13.1 Hz, 1H), 5.63 (d, J=13.1 Hz, 1H), 4.47-4.38 (m, 1H), 4.27 (d, J=8.5 Hz, 1H), 4.11-4.04 (m, 4H), 3.94 (q, J=7.1 Hz, 2H), 3.70 (q, J=4.0 Hz, 1H), 3.38 (d, J=7.4 Hz, 1H), 2.37 (ddd, J=14.0, 8.2, 5.9 Hz, 1H), 2.22 (ddd, J=15.8, 8.5, 5.8 Hz, 1H), 1.95-1.86 (m, 1H), 1.77-1.70 (m, 1H), 1.62 (q, J=6.4, 6.0 Hz, 2H), 1.54 (dt, J=6.7, 3.5 Hz, 2H), 1.30 (t, J=7.1 Hz, 3H), 1.19 (d, J=6.9 Hz, 3H).

### Step 2: Preparation of Intermediate 102c

Intermediate 102b (0.45 g), dichloromethane (10 mL), and hydrochloric acid (0.87 mL, 4 mol/L) were added to a reaction flask in sequence, and the mixture was reacted at room temperature for 4 h. 50 mL of an aqueous NaHCO₃ solution and 50 mL of dichloromethane were added to the reaction solution. The organic phase was separated, dried over anhydrous sodium sulfate, filtered, and concentrated. The concentrate was separated and purified by silica gel column chromatography to give intermediate 102c (0.24 g).

MS(ESI, [M+H]⁺) m/z: 343.3

¹H NMR (500 MHz, DMSO-d₆) δ 7.90 (d, J=9.1 Hz, 1H), 7.67 (d, J=3.1 Hz, 1H), 7.50-7.44 (m, 1H), 6.64 (d, J=3.1 Hz, 1H), 4.56-4.48 (m, 2H), 4.26 (s, 2H), 4.14 (d, J=7.1 Hz, 2H), 3.94 (p, J=3.1 Hz, 1H), 2.16 (qd, J=12.9, 3.6 Hz, 2H), 1.82 (dt, J=9.5, 3.1 Hz, 2H), 1.78-1.70 (m, 4H), 1.20-1.17 (m, 3H).

### Step 3: Preparation of Intermediate 102d

Intermediate 102c (0.25 g), acrylamide (0.057 g), and anhydrous tetrahydrofuran (10 mL) were added to a reaction flask in sequence. Potassium tert-butoxide (0.09 g) was slowly added under an ice bath, and the mixture was reacted for 1 h. The reaction solution was added to 50 mL of a saturated aqueous ammonium chloride solution to quench the reaction, and the mixture was extracted with 50 mL of dichloromethane. The organic phase was separated, dried over anhydrous sodium sulfate, filtered, and concentrated. The concentrate was separated and purified by silica gel column chromatography to give intermediate 102d (0.21 g).

MS(ESI, [M+H]⁺) m/z: 368.2.

¹H NMR (500 MHz, DMSO-d₆) δ 11.18 (s, 1H), 7.92 (d, J=9.2 Hz, 1H), 7.70-7.66 (m, 1H), 7.50-7.46 (m, 1H), 6.56 (d, J=3.1 Hz, 1H), 4.68 (dd, J=11.9, 5.1 Hz, 1H), 4.57-4.50 (m, 2H), 3.94 (q, J=3.0 Hz, 1H), 2.85 (ddd, J=17.4, 12.2, 5.4 Hz, 1H), 2.63 (dt, J=17.3, 4.1 Hz, 1H), 2.43 (td, J=12.7, 4.6 Hz, 1H), 2.27-2.09 (m, 4H), 1.83 (d, J=13.0 Hz, 3H), 1.78-1.69 (m, 2H).

### Step 4: Preparation of Intermediate 102e

Intermediate 102d (2.3 g), acetonitrile (10.00 mL), and IBX oxidant (2.104 g, 7.51 mmol) were added to a reaction flask in sequence, and the mixture was reacted at 80° C. for 1 h. The reaction solution was directly filtered to remove solids, and the filtrate was concentrated to give intermediate 102e (1.63 g).

MS(ESI, [M+H]⁺) m/z: 366.3.

¹H NMR (500 MHz, DMSO-d₆) δ 11.18 (s, 1H), 8.04 (d, J=9.1 Hz, 1H), 7.76-7.71 (m, 1H), 7.54 (d, J=9.1 Hz, 1H), 6.59 (dd, J=8.9, 3.2 Hz, 1H), 5.15 (tt, J=11.1, 4.2 Hz, 1H), 4.73-4.64 (m, 1H), 3.17 (d, J=3.7 Hz, 1H), 2.90-2.75 (m, 3H), 2.63 (dt, J=16.3, 3.7 Hz, 1H), 2.44 (tt, J=12.3, 6.1 Hz, 1H), 2.38-2.29 (m, 3H), 2.24 (tt, J=9.1, 3.9 Hz, 3H).

### Step 5: Preparation of Compounds 102-1 and 102-2

Intermediate 1l (200 mg), intermediate 102e (183 mg), dichloroethane (10 mL), isopropanol (2 mL), and glacial acetic acid (13 mg, 0.209 mmol) were added to a reaction flask. After the mixture was stirred at room temperature for 5 min, sodium cyanoborohydride (53 mg, 0.836 mmol) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give compound 102f (110 mg), which was then purified by C₁₈ reversed-phase column chromatography (10 nM aqueous ammonium acetate solution-acetonitrile=90%:10%-10%:90%) to give compound 102-1 (34 mg) and compound 102-2 (35 mg) in sequence.

### Compound 102-1

Q-TOF (ESI, [M+H]⁺) m/z: 828.4345

¹H NMR (500 MHz, DMSO-d₆) δ 11.19 (d, J=12.7 Hz, 2H), 7.94 (d, J=9.3 Hz, 1H), 7.76 (d, J=2.9 Hz, 1H), 7.72-7.64 (m, 2H), 7.50 (dd, J=8.8, 6.6 Hz, 3H), 7.34 (d, J=2.9 Hz, 1H), 7.17 (d, J=8.2 Hz, 2H), 6.57 (d, J=3.1 Hz, 1H), 4.68 (dd, J=11.9, 5.2 Hz, 1H), 4.54 (dq, J=11.6, 6.7, 5.6 Hz, 1H), 4.36 (d, J=11.4 Hz, 1H), 4.29 (d, J=12.7 Hz, 1H), 3.62 (tt, J=10.6, 4.3 Hz, 1H), 3.26 (dd, J=9.7, 7.2 Hz, 2H), 3.00 (dt, J=41.7, 12.9 Hz, 4H), 2.85 (td, J=12.2, 6.1 Hz, 1H), 2.73 (d, J=3.9 Hz, 3H), 2.63 (dt, J=17.1, 4.1 Hz, 2H), 2.43 (td, J=12.6, 4.5 Hz, 3H), 2.22 (dq, J=13.7, 4.6 Hz, 1H), 2.10-2.03 (m, 2H), 2.02-1.85 (m, 5H), 1.80 (q, J=12.2 Hz, 5H), 1.73-1.51 (m, 6H).

### Compound 102-2

Q-TOF (ESI, [M+H]⁺) m/z: 828.4330

¹H NMR (500 MHz, DMSO-d₆) δ 11.20 (d, J=11.9 Hz, 2H), 7.97 (d, J=9.2 Hz, 1H), 7.76 (s, 1H), 7.67 (s, 2H), 7.51 (dd, J=18.0, 8.5 Hz, 3H), 7.34 (d, J=2.9 Hz, 1H), 7.21 (d, J=8.1 Hz, 2H), 6.59 (d, J=3.0 Hz, 1H), 4.70 (dd, J=11.8, 5.2 Hz, 2H), 4.38 (s, 1H), 4.30 (d, J=13.1 Hz, 1H), 3.67-3.59 (m, 1H), 3.39-3.33 (m, 2H), 3.28-3.21 (m, 3H), 3.00 (dt, J=34.4, 12.0 Hz, 2H), 2.86 (ddd, J=17.3, 12.0, 5.3 Hz, 1H), 2.71 (s, 3H), 2.63 (dt, J=17.1, 4.2 Hz, 1H), 2.43 (td, J=12.2, 11.7, 3.7 Hz, 2H), 2.30 (s, 1H), 2.23 (dt, J=13.6, 4.8 Hz, 2H), 2.14 (d, J=14.1 Hz, 3H), 2.04-1.91 (m, 3H), 1.87-1.73 (m, 7H), 1.68 (s, 3H), 1.57 (d, J=12.3 Hz, 1H).

### Example 103: Synthesis of Compounds 103-1 and 103-2

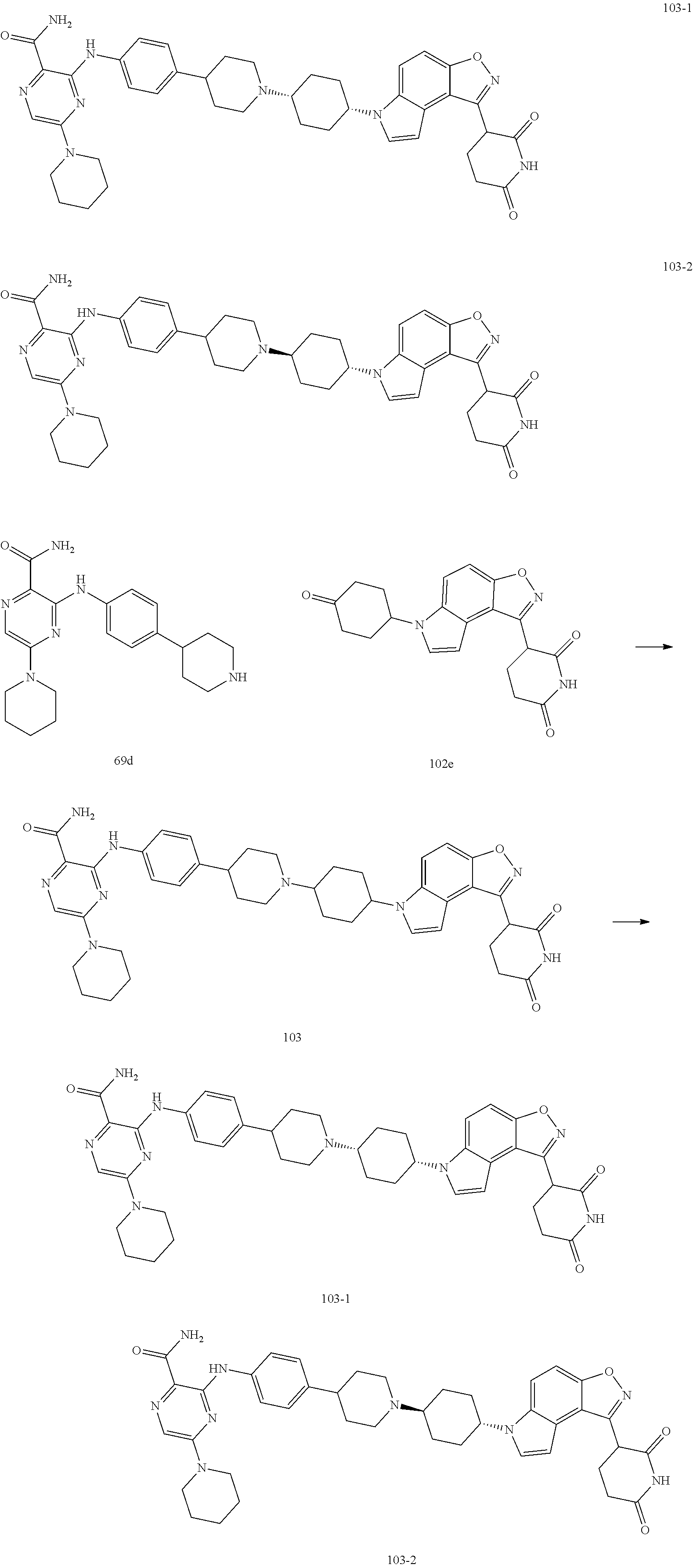

### Step 1: Preparation of Compounds 103-1 and 103-2

Intermediate 69d (220 mg), intermediate 102e (254 mg), dichloroethane (10 mL), isopropanol (2 mL), and glacial acetic acid (17 mg, 0.289 mmol) were added to a reaction flask. After the mixture was stirred at room temperature for 5 min, sodium cyanoborohydride (73 mg, 1.156 mmol) was added, and the mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography to give compound 103 (180 mg), which was then purified by C₁₈ reversed-phase column chromatography (10 nM aqueous ammonium acetate solution-acetonitrile=90%:10%-10%:90%) to give compound 103-1 (68 mg) and compound 103-2 (48 mg) in sequence.

### Compound 103-1

Q-TOF (ESI, [M+H]⁺) m/z: 730.3833

¹H NMR (500 MHz, DMSO-d₆) δ 11.30 (s, 1H), 11.19 (s, 1H), 7.97 (d, J=9.2 Hz, 1H), 7.74 (d, J=2.8 Hz, 1H), 7.67 (s, 2H), 7.55 (d, J=8.1 Hz, 2H), 7.49 (d, J=9.0 Hz, 1H), 7.32 (d, J=2.8 Hz, 1H), 7.23 (d, J=8.2 Hz, 2H), 6.58 (d, J=3.1 Hz, 1H), 4.69 (dt, J=18.4, 9.2 Hz, 2H), 3.69 (t, J=5.4 Hz, 4H), 3.20 (d, J=22.9 Hz, 2H), 2.86 (ddd, J=17.2, 12.0, 5.4 Hz, 1H), 2.63 (dt, J=17.2, 4.2 Hz, 1H), 2.44 (tt, J=12.4, 6.4 Hz, 2H), 2.30 (s, 1H), 2.28-2.20 (m, 2H), 2.15 (dd, J=29.5, 11.9 Hz, 4H), 1.95 (s, 2H), 1.84-1.75 (m, 4H), 1.67 (q, J=5.9 Hz, 5H), 1.60 (t, J=5.5 Hz, 4H).

### Compound 103-2

Q-TOF (ESI, [M+H]⁺) m/z: 730.3840

¹H NMR (500 MHz, DMSO-d₆) δ 11.30 (s, 1H), 11.18 (s, 1H), 7.94 (d, J=9.2 Hz, 1H), 7.74 (d, J=3.0 Hz, 1H), 7.72-7.65 (m, 2H), 7.52 (dd, J=19.2, 8.6 Hz, 3H), 7.32 (d, J=2.9 Hz, 1H), 7.20 (d, J=8.2 Hz, 2H), 6.57 (d, J=3.1 Hz, 1H), 4.68 (dd, J=11.9, 5.2 Hz, 1H), 4.54 (s, 1H), 3.68 (t, J=5.3 Hz, 4H), 2.91 (s, 2H), 2.85 (ddd, J=17.3, 12.1, 5.4 Hz, 1H), 2.63 (dt, J=17.2, 4.2 Hz, 1H), 2.43 (td, J=12.5, 4.4 Hz, 2H), 2.22 (dq, J=13.8, 4.9 Hz, 1H), 2.06 (s, 2H), 2.04-1.94 (m, 2H), 1.90 (d, J=11.2 Hz, 5H), 1.81 (s, 3H), 1.71-1.63 (m, 4H), 1.60 (q, J=5.6 Hz, 4H).

### Example 104: Synthesis of Compound 104

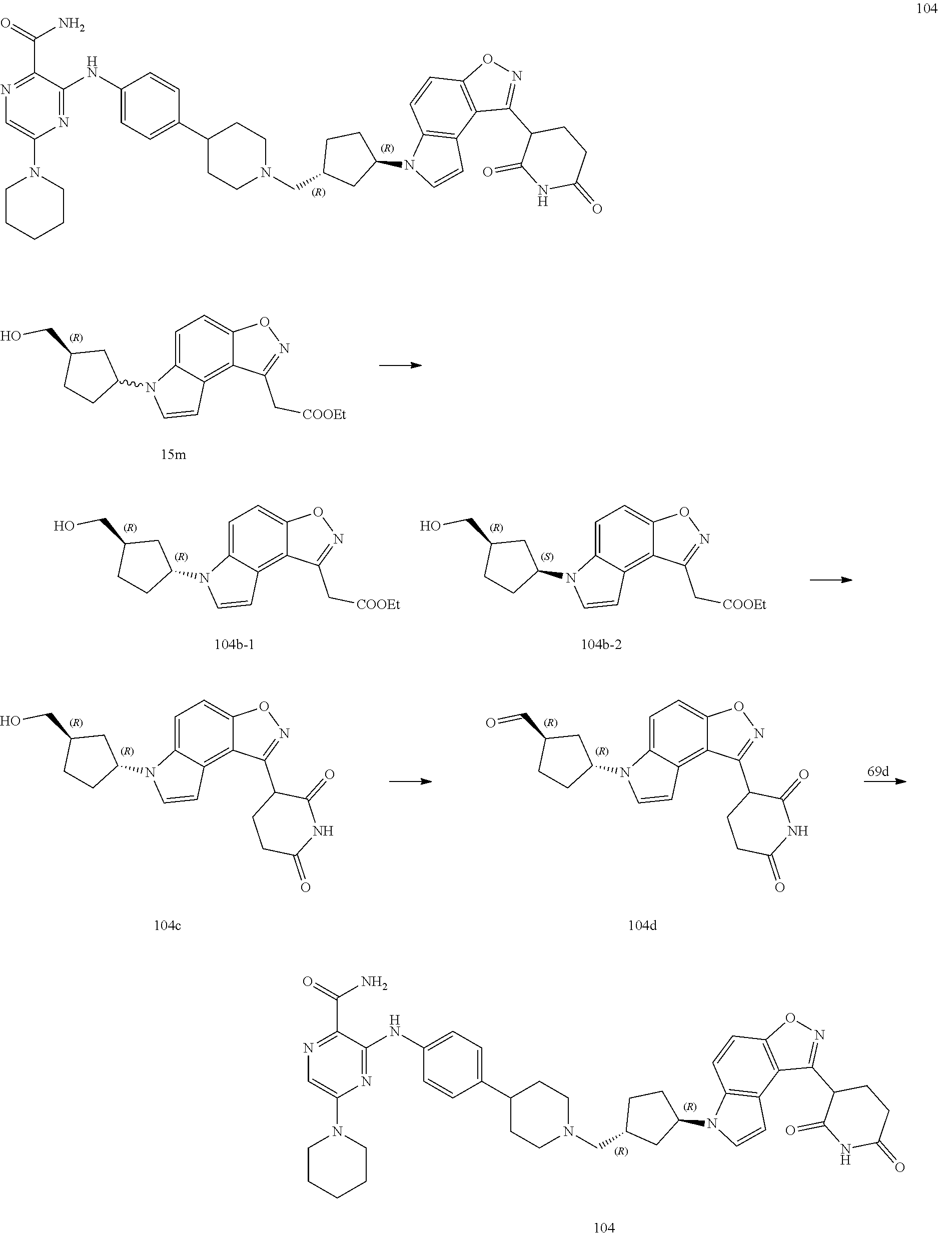

### Step 1: Preparation of Intermediates 104b-1 and 104b-2

Intermediate 15m was separated by high performance liquid chromatography to give intermediates 104b-1 (0.77 g) and 104b-2 (1.51 g) in sequence. The conditions of preparative chromatography were as follows: Instrument and preparative column: YMC K-prep Lab100g high pressure preparative chromatograph was used, and the preparative column was YMC Sil SLG12S11-2530 (30×250 mm, 10 μm). Mobile phase system: ethanol/n-hexane, isocratic elution: ethanol/n-hexane=60/40.

### Intermediate 104b-1

MS(ESI, [M+H]⁺) m/z: 343.3.

¹H NMR (500 MHz, DMSO-d₆) δ 7.91-7.84 (m, 1H), 7.68 (d, J=3.1 Hz, 1H), 7.47 (d, J=9.1 Hz, 1H), 6.65 (d, J=3.1 Hz, 1H), 5.05 (p, J=7.1 Hz, 1H), 4.61 (t, J=5.3 Hz, 1H), 4.26 (s, 2H), 4.14 (q, J=7.1 Hz, 2H), 3.40 (ddd, J=6.7, 5.2, 1.5 Hz, 2H), 2.42-2.31 (m, 1H), 2.22 (tdd, J=13.3, 9.0, 6.0 Hz, 1H), 2.01-1.89 (m, 4H), 1.46 (tdd, J=13.4, 7.5, 4.5 Hz, 1H), 1.18 (td, J=7.1, 3.8 Hz, 3H).

### Intermediate 104b-2

MS(ESI, [M+H]⁺) m/z: 343.2.

¹H NMR (500 MHz, DMSO-d₆) δ 7.90 (d, J=9.0 Hz, 1H), 7.72 (d, J=3.2 Hz, 1H), 7.46 (d, J=9.1 Hz, 1H), 6.65 (d, J=3.1 Hz, 1H), 5.02 (dq, J=9.5, 7.6 Hz, 1H), 4.62 (t, J=5.2 Hz, 1H), 4.26 (s, 2H), 4.16-4.12 (m, 2H), 3.44 (ddd, J=6.5, 5.2, 1.6 Hz, 2H), 2.32 (dt, J=12.4, 7.3 Hz, 1H), 2.20 (dddd, J=25.0, 12.5, 8.0, 6.0 Hz, 2H), 1.97-1.75 (m, 2H), 1.72-1.59 (m, 2H), 1.18 (t, J=7.1 Hz, 3H).

### Step 2: Preparation of Intermediate 104c

Intermediate 104b-1 (0.63 g), acrylamide (0.128 g), and anhydrous tetrahydrofuran (20 mL) were added to a reaction flask in sequence. Potassium tert-butoxide (0.203 g, 1.805 mmol) was slowly added under an ice bath, and the mixture was reacted for 1 h. The reaction solution was added to 100 mL of a saturated aqueous ammonium chloride solution to quench the reaction, and the mixture was extracted with 100 mL of dichloromethane. The organic phase was separated, dried over anhydrous sodium sulfate, filtered, and concentrated. The concentrate was separated and purified by silica gel column chromatography to give intermediate 104c (0.45 g).

MS(ESI, [M+H]⁺) m/z: 368.2.

¹H NMR (500 MHz, DMSO-d₆) δ 11.18 (s, 1H), 7.89 (d, J=9.2 Hz, 1H), 7.69 (d, J=3.1 Hz, 1H), 7.49 (d, J=9.1 Hz, 1H), 6.56 (d, J=3.1 Hz, 1H), 5.05 (p, J=7.1 Hz, 1H), 4.69 (dd, J=11.9, 5.1 Hz, 1H), 4.64-4.57 (m, 1H), 3.40 (ddd, J=6.7, 5.3, 1.5 Hz, 2H), 2.85 (ddd, J=17.3, 12.1, 5.4 Hz, 1H), 2.63 (dt, J=17.3, 4.2 Hz, 1H), 2.44 (qd, J=12.4, 4.4 Hz, 1H), 2.35 (dq, J=13.4, 6.8, 6.3 Hz, 1H), 2.27-2.18 (m, 2H), 2.04-1.89 (m, 4H), 1.47 (tt, J=11.9, 3.8 Hz, 1H).

### Step 3: Preparation of Intermediate 104d

Intermediate 104c (0.45 g), acetonitrile (20.00 mL), and IBX oxidant (0.514 g) were added to a reaction flask in sequence, and the mixture was reacted at 80° C. for 1 h. The reaction solution was directly filtered, and the filtrate was concentrated to give intermediate 104d (0.56 g).

MS(ESI, [M+H]⁺) m/z: 366.3.

### Step 4: Preparation of Compound 104

Intermediate 69d (400 mg), intermediate 104d (499 mg), dichloroethane (20 mL), and isopropanol (4 mL) were added to a reaction flask, and then sodium triacetoxyborohydride (334 mg, 1.577 mmol) was added. The mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography and C₁₈ reversed-phase column chromatography to give compound 104 (266 mg).

Q-TOF (ESI, [M+H]⁺) m/z: 730.3839

¹H NMR (500 MHz, DMSO-d₆) δ 11.28 (s, 1H), 11.18 (s, 1H), 7.91 (d, J=9.1 Hz, 1H), 7.72 (dd, J=10.2, 3.0 Hz, 2H), 7.66 (s, 1H), 7.50 (dd, J=8.8, 6.8 Hz, 3H), 7.31 (d, J=2.9 Hz, 1H), 7.18 (d, J=8.3 Hz, 2H), 6.57 (d, J=3.1 Hz, 1H), 5.12 (p, J=7.1 Hz, 1H), 4.69 (dd, J=11.9, 5.1 Hz, 1H), 3.67 (t, J=5.3 Hz, 4H), 3.00 (s, 2H), 2.85 (ddd, J=17.3, 12.1, 5.4 Hz, 1H), 2.63 (dt, J=17.1, 4.2 Hz, 1H), 2.43 (td, J=12.5, 4.5 Hz, 3H), 2.34 (s, 2H), 2.25 (tp, J=14.0, 5.1, 4.3 Hz, 2H), 1.98 (tdd, J=22.0, 13.4, 6.9 Hz, 6H), 1.73 (s, 2H), 1.68-1.53 (m, 8H), 1.44 (dq, J=11.9, 8.0 Hz, 1H).

### Example 105: Synthesis of Compound 105

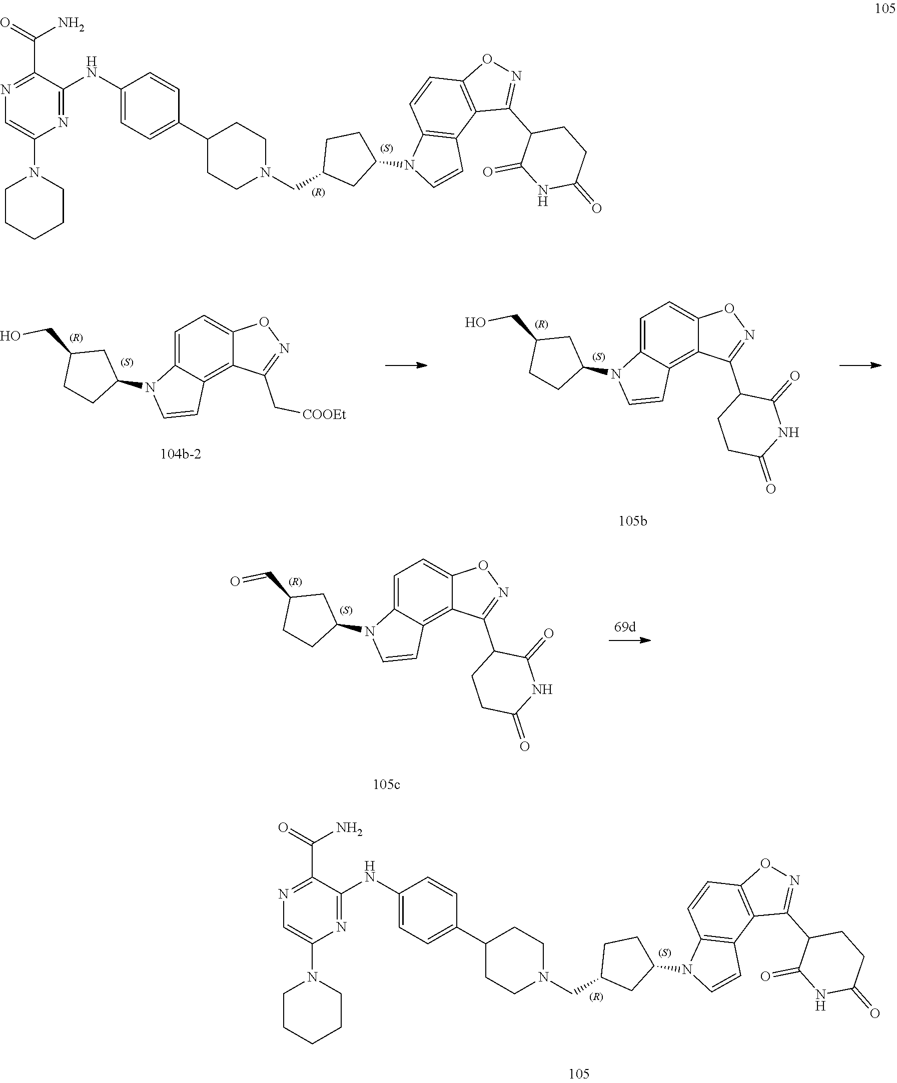

### Step 1: Preparation of Intermediate 105b

Intermediate 104b-2 (0.35 g), acrylamide (0.078 g), and anhydrous tetrahydrofuran (20 mL) were added to a reaction flask in sequence. Potassium tert-butoxide (0.123 g, 1.1 mmol) was slowly added under an ice bath, and the mixture was reacted for 1 h. The reaction solution was added to 100 mL of a saturated aqueous ammonium chloride solution to quench the reaction, and the mixture was extracted with 100 mL of dichloromethane. The organic phase was separated, dried over anhydrous sodium sulfate, filtered, and concentrated. The concentrate was separated and purified by silica gel column chromatography to give intermediate 105b (0.27 g).

MS(ESI, [M+H]⁺) m/z: 368.2.

¹H NMR (500 MHz, DMSO-d₆) δ 11.18 (s, 1H), 7.91 (d, J=9.2 Hz, 1H), 7.72 (d, J=3.2 Hz, 1H), 7.48 (d, J=9.1 Hz, 1H), 6.57 (d, J=3.1 Hz, 1H), 5.02 (p, J=8.1 Hz, 1H), 4.69 (dd, J=11.9, 5.1 Hz, 1H), 4.62 (t, J=5.2 Hz, 1H), 3.46-3.41 (m, 2H), 2.85 (ddd, J=17.4, 12.1, 5.4 Hz, 1H), 2.63 (dt, J=17.2, 4.2 Hz, 1H), 2.44 (qd, J=12.2, 4.4 Hz, 1H), 2.31 (dt, J=12.2, 7.3 Hz, 1H), 2.21 (ddt, J=26.5, 12.5, 5.5 Hz, 3H), 1.94-1.79 (m, 2H), 1.64 (tdd, J=12.4, 5.9, 3.3 Hz, 2H).

### Step 2: Preparation of Intermediate 105c

Intermediate 105b (0.27 g), acetonitrile (20.00 mL), and IBX oxidant (0.309 g) were added to a reaction flask in sequence, and the mixture was reacted at 80° C. for 1 h. The reaction solution was directly filtered, and the filtrate was concentrated to give intermediate 105c (0.25 g).

MS(ESI, [M+H]⁺) m/z: 366.3.

### Step 3: Preparation of Compound 105

Intermediate 69d (270 mg), intermediate 105c (389 mg), dichloroethane (20 mL), and isopropanol (4 mL) were added to a reaction flask, and then sodium triacetoxyborohydride (301 mg, 1.419 mmol) was added. The mixture was stirred overnight at room temperature. After the reaction was completed, 100 mL of water was added to the reaction solution, and the mixture was extracted twice with 100 mL of dichloromethane. The extracts were combined, dried over anhydrous sodium sulfate, filtered under vacuum, and concentrated, and the residue was purified by silica gel column chromatography and C₁₈ reversed-phase column chromatography to give compound 105 (181 mg).

Q-TOF (ESI, [M+H]⁺) m/z: 730.3855

¹H NMR (500 MHz, DMSO-d₆) δ 11.28 (s, 1H), 11.18 (s, 1H), 7.91 (d, J=9.2 Hz, 1H), 7.74 (t, J=3.8 Hz, 2H), 7.66 (s, 1H), 7.50 (dd, J=12.1, 8.6 Hz, 3H), 7.31 (d, J=2.9 Hz, 1H), 7.18 (d, J=8.1 Hz, 2H), 6.59 (d, J=3.1 Hz, 1H), 5.04 (q, J=8.2 Hz, 1H), 4.69 (dd, J=11.8, 5.1 Hz, 1H), 3.67 (t, J=5.2 Hz, 4H), 3.01 (s, 2H), 2.85 (ddd, J=17.4, 12.0, 5.4 Hz, 1H), 2.63 (dt, J=17.2, 4.2 Hz, 1H), 2.49-2.29 (m, 6H), 2.23 (dq, J=14.3, 5.9, 5.1 Hz, 2H), 2.09-1.88 (m, 4H), 1.75 (s, 2H), 1.65 (q, J=6.0, 5.6 Hz, 4H), 1.58 (h, J=7.1, 5.6 Hz, 6H).

### Test Example 1: Effect of Compounds on BTK Degradation in OCI-LY10 Cells

The detection was carried out using the Total-BTK Kits (cisbio, 63ADK064PEH), and 4× Supplemented Lysis buffer was prepared using the Blocking reagent stock solution (100×) and Lysis buffer stock solution (4×). Premixed antibody solutions were prepared using the Detection buffer, Total-BTK d2 antibody, and Total-BTK Eu Cryptate antibody.

OCI-LY10 cells in a good growth state were collected and added to a centrifuge tube, adjusted to a cell density of 4.17×10⁶ cells/mL, and seeded in a 384-well plate (12 μL/well). The cells were incubated overnight in a cell incubator. Compounds were added using a nanoliter pipettor such that the final concentrations of the compounds were 100 nM-0.098 nM; the addition was performed in duplicate, and meanwhile, a control was set. After another 24 (or 6) h of incubation in the cell incubator, the 4× Supplemented Lysis buffer was added at 4 μL/well to lyse the cells. After the cells were shaken for 40 min at room temperature, the Premixed antibody solutions were added at 4 μL/well, and the cells were left to stand overnight at room temperature. Fluorescence values of the cells were detected at 665 nm/620 nm using an Envision microplate reader, the four-parameter analysis was performed, the dose-response curves were fit, and DC₅₀ (the concentration of the drug when the degradation rate reaches 50%) and Dmax (the maximum degradation rate) were calculated. The experimental results are shown in Table 1.

TABLE 1

| | | | | BTK | | BTK | | BTK |
|---|---|---|---|---|---|---|---|---|
| | | | | degradation | | degradation | | degradation |
| | | | | in | | in | | in |
| | OCI-LY10 cell | | | OCI-LY10 | | OCI-LY10 | | OCI-LY10 |
| | BTK degradation | | | cells | | cells | | cells |
| Compound | DC₅₀ (nM) | Dmax (%) | Compound | Dmax (%) | Compound | Dmax (%) | Compound | Dmax (%) |
| 1 | <0.098 | 99 | 24 | >90 | 52 | >90 | 88 | >90 |
| 2 | <0.098 | 99 | 26 | >90 | 54 | >90 | 89 | >90 |
| 3 | <0.098 | 99 | 28 | >90 | 55 | >90 | 90 | >90 |
| 6 | <0.098 | 99 | 29 | >90 | 56 | >90 | 93 | >90 |
| 7 | <0.098 | 99 | 30 | >90 | 57 | >90 | 94 | >90 |
| 8 | <0.098 | 99 | 31 | >90 | 58 | >90 | 96 | >90 |
| 9 | <0.098 | 99 | 33 | >90 | 59 | >90 | 97 | >90 |
| 10 | <0.098 | 99 | 35 | >90 | 62 | >90 | 100 | >90 |
| 11 | <0.098 | 99 | 40 | >90 | 72 | >90 | 102-1 | >90 |
| 12 | 0.098 | 99 | 41 | >90 | 76 | >90 | 103-2 | >90 |
| 13 | <0.098 | 100 | 44 | >90 | 77 | >90 | 98-1 | >90 |
| 14 | <0.098 | 100 | 45 | >90 | 78 | >90 | 98-2 | >90 |
| 15 | <0.098 | 100 | 46 | >90 | 79 | >90 | 99-1 | >90 |
| 27 | <0.098 | 97 | 47 | >90 | 80 | >90 | | |
| 34 | <0.098 | 97 | 48 | >90 | 81 | >90 | | |
| 36 | <0.098 | 95 | 49 | >90 | 82 | >90 | | |
| 53 | <0.098 | 95.97 | 50 | >90 | 86 | >90 | | |
| 102-2 | <0.098 | 97.11 | 51 | >90 | 87 | >90 | | |

### Test Example 2: Inhibitory Activity on In Vitro Cell Proliferation

### 2.1 Assay for Inhibitory Activity on OCI-LY10 Cell Proliferation

OCI-LY10 cells in a good growth state were collected and added to a centrifuge tube, adjusted to a cell density of 1×105 cells/mL, and seeded in a 96-well plate (100 μL/well). The cells were incubated overnight in a cell incubator. Compounds were added using a nanoliter pipettor such that the final concentrations of the compounds were 5000 nM-0.31 nM; the addition was performed in duplicate, and meanwhile, a control was set. After another 72 h of incubation in the cell incubator, 10 μL of CCK-8 (CK04, PP799) was added. After 2.5 h of incubation in the cell incubator, absorbance values were detected at 450 nm using an Envision microplate reader, the four-parameter analysis was performed, the dose-response curves were fit, and IC50 was calculated.

2.2 Assay for inhibitory activity on TMD8-BTK(C481S) cell proliferation

TMD8-BTK(C481S) cells in a good growth state were collected and added to a centrifuge tube, adjusted to a cell density of 8×10⁴ cells/mL, and seeded in a 96-well plate (100 μL/well). The cells were incubated overnight in a cell incubator. Compounds were added using a nanoliter pipettor such that the final concentrations of the compounds were 5000 nM-0.31 nM; the addition was performed in duplicate, and meanwhile, a control was set. After another 72 h of incubation in the cell incubator, 10 μL of CCK-8 (CK04, PP799) was added. After 4 h of incubation in the cell incubator, absorbance values were detected at 450 nm using an Envision microplate reader, the four-parameter analysis was performed, the dose-response curves were fit, and IC50 was calculated.

### 2.3 Assay for Inhibitory Activity on OCI-LY10-BTK(C481S) Cell Proliferation

OCI-LY10(C481S) cells in the logarithmic growth phase were collected and added to a centrifuge tube, adjusted to a cell density of 5×10⁴ cells/mL, and seeded in a 96-well plate (100 μL/well). Meanwhile, compounds were added using a nanoliter pipettor such that the final concentrations of the compounds were 5000 nM-0.31 nM; the addition was performed in duplicate, and meanwhile, a control was set. After another 72 h of incubation in the cell incubator, the detection reagent CCK-8 (manufacturer: Dojindo Laboratories, Beijing; 10 μL/well) was added. After 3 h of incubation in the cell incubator, absorbance values were detected at 450 nm using a PerkinElmer Envision microplate reader, the four-parameter analysis was performed, the dose-response curves were fit, and IC50 was calculated. The results are shown in Table 2.

TABLE 2

| | OCI-LY10 | TMD8-BTK^{C481S} | OCI-LY10- |
|---|---|---|---|
| Compound | IC50 (nM) | IC50 (nM) | BTK^{C481S} |
| 1 | <0.31 | 35 | |
| 2 | | 18 | |
| 3 | <0.31 | | |
| 4 | | 27 | |
| 6 | <0.31 | 27 | |
| 7 | <0.31 | | |
| 8 | <0.31 | 6.9 | |
| 9 | <0.31 | 1.2 | |
| 10 | <0.31 | 2.4 | |
| 11 | <0.31 | 14 | |
| 13 | <0.31 | | <0.31 |
| 14 | <0.31 | | <0.31 |
| 15 | <0.31 | | <0.31 |
| 24 | <0.31 | | 0.5 |
| 26 | <0.31 | | 0.26 |
| 27 | <0.31 | | 0.062 |
| 28 | <0.31 | | 0.29 |
| 29 | <0.31 | | 0.35 |
| 30 | <0.31 | | 0.44 |
| 33 | <0.31 | | 0.37 |
| 34 | <0.31 | | 0.19 |
| 35 | <0.31 | | 0.12 |
| 36 | <0.31 | | 0.024 |
| 40 | | | 0.51 |
| 41 | <0.31 | | 0.34 |
| 44 | | | 0.56 |
| 46 | | | 0.66 |
| 47 | | | 0.62 |
| 48 | | 5.7 | |
| 49 | | 6.8 | |
| 50 | | 4 | |
| 51 | | 4.7 | |
| 52 | | 3.7 | |
| 53 | <0.31 | 2.8 | |
| 54 | | 9.7 | |
| 55 | | 6.1 | |
| 56 | | 33 | |
| 58 | | 21 | |
| 59 | | 12 | |
| 60 | | 9.4 | |
| 61 | | 13 | |
| 62 | <0.31 | 8.2 | |
| 67 | | 32 | |
| 69 | | 5.7 | |
| 70 | | 15 | |
| 72 | | 2.6 | |
| 74 | | 23 | |
| 75 | | 9.8 | |
| 76 | | 11 | |
| 77 | <0.31 | 23 | |
| 79 | <0.31 | 12 | |
| 80 | <0.31 | 11 | |
| 81 | <0.31 | 11 | |
| 82 | <0.31 | 11 | |
| 87 | <0.31 | 11 | |
| 88 | | 29 | |
| 89 | <0.31 | 20 | |
| 90 | <0.31 | 1.2 | |
| 93 | | | 0.7 |
| 94 | <0.31 | | 0.53 |
| 96 | <0.31 | | 0.31 |
| 97 | | | 0.78 |
| 100 | <0.31 | 0.57 | |
| 104 | | 16 | |
| 105 | | 6.9 | |

### Test Example 3: In Vitro Kinase Inhibition and Selectivity

### 3.1 Inhibitory Activity on BTK(WT)

The Assay Buffer was prepared, and the composition of the Assay Buffer was 50 mM Hepes (Gibco, 15630), 10 mM MgCl₂, 2 mM DTT, 1 mM EGTA, and 0.010% Tween 20. BTK(WT) kinase (Life, PR5442A), ATP (Sigma, A7699), and ULight-poly GT (PE, TRF0100-M) working solutions were prepared using the Assay Buffer. EDTA and Eu-labeled anti-phosphotyrosine (PT66) antibody (PE, AD0069) working solutions were prepared using the Detection Buffer (PE, CR97-100). 6 μL of BTK(WT) kinase at the corresponding concentration (final concentration: 0.003 ng/L) was added to the compound groups and the control group, while 6 μL of the Assay Buffer was added to the blank group, and then the compounds (with the concentration set to 1000 nM at the maximum, 4-fold dilution, 7 concentration gradients) were each added using a nanoliter pipettor. The mixture was incubated at room temperature for 30 min, and then 4 μL of the mixture of ATP (final concentration: 10 μM) and ULight-poly GT (final concentration: 100 nM) was added. After incubation at room temperature for 2 h, 5 μL of EDTA (final concentration: 10 mM) was added to stop the reaction, and finally 5 μL of antibody (final concentration: 2 nM) was added. After incubation at room temperature for 1 h, signal values were detected at 665/615 nM, the four-parameter analysis was performed, the dose-response curves were fit, and IC50 was calculated.

### 3.2 Inhibitory Activity on BTK(C481S)

The Assay Buffer was prepared, and the composition of the Assay Buffer was 50 mM Hepes (Gibco, 15630), 10 mM MgCl₂, 2 mM DTT, 1 mM EGTA, and 0.010% Tween 20. BTK(C481S) kinase (Carna Biosciences, 08-547), ATP (Sigma, A7699), and ULight-poly GT (PE, TRF0100-M) working solutions were prepared using the Assay Buffer. EDTA and Eu-labeled anti-phosphotyrosine (PT66) antibody (PE, AD0069) working solutions were prepared using the Detection Buffer. 6 μL of BTK(C481S) kinase at the corresponding concentration (final concentration: 0.005 ng/μL) was added to the compound groups and the control group, while 6 μL of the Assay Buffer was added to the blank group, and then the compounds (with the concentration set to 1000 nM at the maximum, 4-fold dilution, 7 concentration gradients) were added using a nanoliter pipettor. The mixture was incubated at room temperature for 30 min, and then 4 μL of the mixture of ATP (final concentration: 10 μM) and ULight-poly GT (final concentration: 100 nM) was added. After incubation at room temperature for 2 h, 5 μL of EDTA (final concentration: 10 mM) was added to stop the reaction, and finally 5 μL of antibody (final concentration: 2 nM) was added. After incubation at room temperature for 1 h, signal values were detected at 665/615 nM, the four-parameter analysis was performed, the dose-response curves were fit, and IC50 was calculated.

3.3 Inhibitory activity on EGFR

The Assay Buffer was prepared, and the composition of the Assay Buffer was 50 mM Hepes (Gibco, 15630), 10 mM MgCl₂, 2 mM DTT, 1 mM EGTA, and 0.010% Tween 20. EGFR kinase (Carna, 08-115), ATP (Sigma, A7699), and ULight-poly GT (PE, TRF0100-M) working solutions were prepared using the Assay Buffer. EDTA and Eu-labeled anti-phosphotyrosine (PT66) antibody (PE, AD0069) working solutions were prepared using the Detection Buffer. 6 μL of EGFR kinase at the corresponding concentration (final concentration: 0.006 ng/μL) was added to the compound groups and the control group, while 6 μL of the Assay Buffer was added to the blank group, and then the compounds (with the concentration set to 1000 nM at the maximum, 4-fold dilution, 7 concentration gradients) were added using a nanoliter pipettor. The mixture was incubated at room temperature for 30 min, and then 4 μL of the mixture of ATP (final concentration: 5 μM) and ULight-poly GT (final concentration: 100 nM) was added. After incubation at room temperature for 2 h, 5 μL of EDTA (final concentration: 10 mM) was added to stop the reaction, and finally 5 μL of antibody (final concentration: 2 nM) was added. After incubation at room temperature for 1 h, signal values were detected at 665/615 nM, the four-parameter analysis was performed, the dose-response curves were fit, and IC50 was calculated.

### 3.4 Inhibitory Activity on TEC

The Assay Buffer was prepared, and the composition of the Assay Buffer was 50 mM Hepes (Gibco, 15630), 10 mM MgCl₂, 2 mM DTT, 1 mM EGTA, and 0.010% Tween 20. TEC kinase (Carna, 08-115), ATP (Sigma, A7699), and ULight-poly GT (PE, TRF0100-M) working solutions were prepared using the Assay Buffer. EDTA and Eu-labeled anti-phosphotyrosine (PT66) antibody (PE, AD0069) working solutions were prepared using the Detection Buffer. 6 μL of TEC kinase at the corresponding concentration (final concentration: 0.01 ng/μL) was added to the compound groups and the control group, while 6 μL of the Assay Buffer was added to the blank group, and then the compounds (with the concentration set to 1000 nM at the maximum, 4-fold dilution, 7 concentration gradients) were added using a nanoliter pipettor. The mixture was incubated at room temperature for 30 min, and then 4 μL of the mixture of ATP (final concentration: 10 μM) and ULight-poly GT (final concentration: 100 nM) was added. After incubation at room temperature for 2 h, 5 μL of EDTA (final concentration: 10 mM) was added to stop the reaction, and finally 5 μL of antibody (final concentration: 2 nM) was added. After incubation at room temperature for 1 h, signal values were detected at 665/615 nM, the four-parameter analysis was performed, the dose-response curves were fit, and IC50 was calculated. The results are shown in Table 3.

TABLE 3

| | BTK | BTK^{C481S} | EGFR |
|---|---|---|---|
| Compound | IC50 (nM) | | |
| 10 | 7.1 | 19.6 | 3317 |
| 27 | 0.96 | 9 | 109 |
| 33 | 11 | 22 | 785 |
| 50 | 0.42 | 0.81 | 69 |
| 59 | 5.4 | 9.1 | 279 |

### Test Example 4: In Vitro Liver Microsomal Stability

Liver microsome incubation samples were prepared by mixing a PBS buffer (pH 7.4), a liver microsome solution (0.5 mg/mL), a test compound, and an NADPH+MgCl₂ solution and incubated at 37° C. and 300 rpm for 1 h. Zero-hour samples were prepared by mixing a PBS buffer (pH 7.4), a liver microsome solution (0.5 mg/mL), and a test compound. An acetonitrile solution containing an internal standard was added to the samples, and supernatants were prepared by protein precipitation, diluted, and then assayed by LC/MS/MS. The results are shown in Table 4.

TABLE 4
In vitro metabolic stability of related compounds
in liver microsomes of various species

| | |
|---|---|
| Mouse liver | Human liver |
| microsome | microsome |

| | |
|---|---|
| Compound | Residuals at 60 min (%) |

| | | |
|---|---|---|
| 7 | 42.7% | |
| 8 | 54.7% | |
| 10 | 65.4% | |
| 13 | 56.41% | |
| 14 | 62.41% | |
| 27 | | 56.0% |
| 33 | 71.0% | 55.0% |
| 50 | 63.1% | |
| 59 | 47.0% | |
| 69 | 85.0% | 80.0% |
| 72 | | 94.0% |
| 79 | 61.19% | |
| 80 | 51.56% | |
| 90 | 54.63% | |
| 93 | 72.0% | 53.0% |
| 94 | 75.0% | 81.0% |

### Test Example 5: Mouse Pharmacokinetics

ICR mice weighing 18-22 g were randomized into groups of 9 after 3-5 days of acclimatization and then intragastrically given the test compound solution at a dose of 10 mg/kg. Plasma samples to be tested were prepared by taking blood from the orbit at time points of 15 min, 30 min, 1 h, 2 h, 3 h, 4 h, 6 h, 8 h, 10 h, and 24 h. 20 μL of each plasma sample to be detected and a standard curve sample were taken, and an acetonitrile solution containing an internal standard was added. Supernatants were obtained by protein precipitation, diluted, and then assayed by LC/MS/MS. Pharmacokinetic parameters were fitted using a non-compartmental model (see Table 5).

TABLE 5

| | Mouse i.v. | | | Mouse i.g. | | | |
|---|---|---|---|---|---|---|---|
| | 1 mpk | | | 10 mpk | | | |
| Compound | AUC (ng*h/mL) | t1/2 (h) | Cl (L/hr/kg) | AUC (ng*h/mL) | t1/2 (h) | Cmax (ng/mL) | F (%) |
| 9 | 2971 | 1.5 | 0.331 | 13697 | 2.6 | 1392 | 46.10% |
| 10 | 6138 | 1.6 | 0.159 | 35589 | 2.9 | 3848 | 57.98% |
| 27 | 1758 | 1.4 | 0.56 | 7687 | 2.4 | 766 | 44.0% |
| 33 | 7987 | 2.6 | 0.13 | 20971 | 3.2 | 1555 | 26.0% |
| 69 | 8033 | 2.6 | 0.12 | 41431 | 7 | 2732 | 51.6% |
| 72 | 1287 | 2.3 | 0.72 | 8301 | 4.6 | 634 | 64.50% |
| 89 | 9703 | 2.8 | 0.103 | 23740 | 3 | 1881 | 24.5% |
| 90 | 8882 | 2.7 | 0.11 | 18944 | 3 | 2550 | 21.0% |

### Test Example 6: Study on In Vivo Drug Efficacy

TMD-8 mouse subcutaneous xenograft tumors: TMD-8 (WT) cells at a concentration of 1×10⁸/mL were inoculated under aseptic conditions into the right side armpit of NOD-SCID mice at 0.1 mL/mouse. After subcutaneous grafting, the animals were divided into groups with 6 animals in each group when the tumor volume reached about 188 mm³ (or 200 mm³):

model group: vehicle, 6 mice; compound administration groups: 30 mg/kg, qd, i.g., 6 mice.

The vehicle or the test compound was intragastrically given at a volume of 4 mL/kg, once daily for 16 consecutive days. The tumor volume was measured 2-3 times a week, and meanwhile, the mice were weighed and the data were recorded; the general behaviors of the animals were observed every day. After all dosing was complete, the animals were sacrificed on day 17, and the tumors were removed and weighed. The results are shown in Table 6.

The tumor volume and the tumor growth inhibition were calculated using the following formulae:

Tumor volume (TV)=(Length×Width²)/2.

Tumor growth inhibition (TGI)=(1−tumor weight of treatment group/tumor weight of model group)×100%.

TABLE 6
Therapeutic effect of compounds
on mouse TMD-8 xenograft tumors

| | TV on | TV on | TV on | TGI |
|---|---|---|---|---|
| | d 0 (mm³) | d 12 (mm³) | d 15 (mm³) | (%) |
| Compound | Mean ± SD | Mean ± SD | Mean ± SD | d 17 |
| Model group | 226 ± 27 | 1347 ± 290 | 1629 ± 252 | — |
| 10 | 226 ± 29 | 235 ± 57 | 209 ± 60 | 97.5% |
| 105 | 226 ± 28 | 253 ± 36 | 229 ± 49 | 95.4% |
| 104 | 226 ± 27 | 286 ± 77 | 269 ± 61 | 96.0% |
| 69 | 226 ± 21 | 246 ± 56 | 199 ± 72 | 96.4% |
| 72 | 226 ± 21 | 246 ± 52 | 122 ± 78 | 97.9% |

### Test Example 7: Binding Activity for CRBN Protein

Reagent: CEREBLON BINDING KITS, Purchased from Cisbio

### Preparation of Working Solutions:

1) Preparation of Human Cereblon WT GST-tagged protein working solution according to requirements of the kit

The Human Cereblon WT GST-tagged protein stock solution was diluted from 45× to ½× with the PROTAC binding Buffer
2) Preparation of GST Eu Cryptate Antibody+Thalidomide-Red reagent working solution according to requirements of the kit
a. preparing 1×GST Eu Cryptate Antibody: the GST Eu Cryptate Antibody stock solution was diluted from 50× to 1× with the PROTAC binding Buffer,
b. preparing 1× Thalidomide-Red reagent: the Thalidomide-Red reagent stock solution was diluted from 50× to 1× with the PROTAC binding Buffer,
c. sufficiently mixing 1×GST Eu Cryptate Antibody and 1× Thalidomide-Red reagent at a ratio of 1:1.

Test process: the whole experiment was carried out at room temperature. A 384-well white low-volume microplate was used; for the experimental groups and the negative group, the ½× Human Cereblon WT GST-tagged protein working solution was added at 5 μL/well, and for the blank group, the PROTAC binding Buffer was added at 5 μL/well. Then, the compounds (at an initial concentration of 10 μM, 7 concentration gradients by 1:3 serial dilution) were added to the experimental groups using an ultra microsyringe. Finally, 10 μL of GST Eu Cryptate Antibody+Thalidomide-Red reagent working solution was added to all groups, and after incubation for 3 h at room temperature, signal values were detected at 665 nm/620 nm using a PerkinElmer Envision HTS multi-label plate reader. The inhibition rate was calculated by the following formula: inhibition rate (%)=(mean value of negative control group−mean value of experimental group)/(mean value of negative control group−mean value of blank group)×100%. The curves were fit using the four-parameter logistic model with the logarithm of the compound concentration as the abscissa and the inhibition rate as the ordinate, and IC₅₀ values were calculated.

The test compounds of the present application have binding activity for CRBN protein (IC₅₀<5 μM).

### Test Example 8: In Vitro Test for IKZF1I/IKZF3/GSPT1 Degradation Levels in Multiple Myeloma Cell

The regulation of the levels of proteins IKZF1/IKZF3/GSPT1 in multiple myeloma cell MM.1S by compounds was studied by using the Western Blot method.

Experimental scheme: MM.1S cells were seeded in a 6-well plate and then treated with the test compounds, and the cells were collected and lysed on ice with RIPA buffer containing a compound protease inhibitor (Roche).

Supernatants were collected by centrifugation and protein quantification was performed (BCA protein assay kit, Thermo). An equal amount of 20 μg of total protein was separated by SDS-PAGE, transferred to a PVDF membrane, and then blocked at room temperature by the addition of 5% skimmed milk powder (prepared with 0.1% TBS-T). Primary antibodies anti-IKZF1, anti-IKZF-3, anti-GSPT1, and anti-Actin were each diluted with 5% BSA (prepared with 0.1% TBS-T) according to a ratio, and the PVDF membrane was incubated in the primary antibody overnight at 4° C. The next day, after incubation with a HRP-labeled secondary antibody at room temperature for a period of time, the band on the membrane was detected using the ECL chemiluminescent substrate (Thermo) to determine whether the compound had the ability to degrade IKZF1/IKZF3/GSPT1 protein.

The test compounds of the present application have the activity in degrading IKZF1, IKZF3, and GSPT1 proteins. The above examples are only preferred examples of the present application and are not intended to limit the scope of the present application. It will be appreciated by those skilled in the art that various modifications and changes may be made based on the examples described above, and any modifications, substitutions, combinations, etc., made within the spirit and conception of the present application shall all fall within the protection scope of the present application.

1. A compound of formula I, a stereoisomer thereof, or a pharmaceutically acceptable salt thereof, 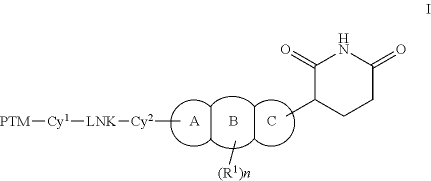
wherein,
ring A is absent or selected from the group consisting of C₅₋₁₀ cycloalkenyl, 5- to 10-membered heterocycloalkenyl, phenyl, and 5- to 6-membered heteroaryl, or
ring B is selected from phenyl;
ring C is selected from the group consisting of isoxazolyl and furanyl;
each R¹ is independently selected from the group consisting of halogen, —OH, —NH₂, —CN, C₁₋₄ alkyl, C₁₋₄ alkoxy, and C₁₋₄ haloalkyl;
n is selected from the group consisting of 0, 1, 2, and 3;
Cy¹ is selected from the group consisting of a bond, C₃-12 cycloalkyl, and 4- to 12-membered heterocycloalkyl, wherein the C₃-12 cycloalkyl or 4- to 12-membered heterocycloalkyl is optionally substituted with one or more R^{a};
LNK is selected from the group consisting of a bond, C₁-12 alkylene, and C₁-12 heteroalkylene;
Cy² is absent or selected from the group consisting of C₃-12 cycloalkyl and 4- to 12-membered heterocycloalkyl, wherein the cycloalkyl or heterocycloalkyl is optionally substituted with one or more R^{b};
each R^{a} and each R^{b} are independently selected from the group consisting of halogen, —OH, —NH₂, —CN, C₁₋₄ alkyl, C₁₋₄ alkoxy, C₁₋₄ haloalkyl, C₁₋₄ alkylamino, and di-C₁₋₄ alkylamino;
PTM is selected from the group consisting of drugs and derivatives thereof that bind to a target protein.

2-43. (canceled)

44. The compound, the stereoisomer thereof, or the pharmaceutically acceptable salt thereof according to claim 1, wherein PTM is selected from the group consisting of drugs and derivatives thereof that act on AR, ER, kinase, phosphatase, MDM2, human BET bromodomain protein, Hsp90, HDAC, human lysine methyltransferase, RAF receptor, FKBP, angiogenic factor, immunosuppression-related receptor or protein, arene receptor, thyroid hormone receptor, HIV protease, HIV integrase, HCV protease, HBV protease, or acyl protein thioesterase 1 and/or 2;
optionally, PTM is selected from the group consisting of drugs and derivatives thereof that act on ALK, BET, CDK, PARP, EGFR, 7-secretase, CBFβ-SMMHC, WEE1, MEK, BCR-ABL, MET, RAS, BTK, VEGFR, JAK, HER2, HDAC, Akt, PI3K, mTOR, AR, ER, PDEδ, SRC, MDM2, RAF, IRAK4, STAT3 and c-Myc; or
PTM is selected from the group consisting of drugs and derivatives thereof that act on ALK, BRD4, CDK4/6, PARP, EGFR, 7-secretase, CBFβ-SMMHC, WEE1, MEK, BCR-ABL, MET, KRAS, EGFR, BTK, AR, ER, PDEδ, JAK, MDM2 or RAF; or
PTM is selected from the group consisting of drugs and derivatives thereof that act on BTK or WEE1; or
PTM is selected from the group consisting of drugs and derivatives thereof that act on BTK.

45. A compound of formula II-1 or formula I″, a stereoisomer thereof, or a pharmaceutically acceptable salt thereof, 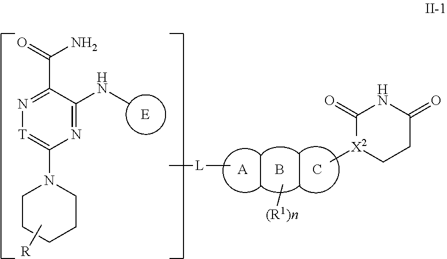
wherein,
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl;
ring E is selected from the group consisting of phenyl, benzocycloalkenyl, and benzoheterocycloalkenyl;
X² is selected from the group consisting of CH and N;
L is selected from a connecting group,
wherein ring A, ring B, ring C, R¹, or n is as defined in claim 1; or 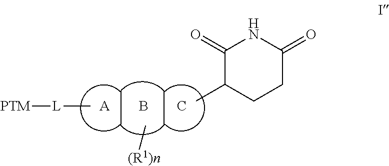
wherein, 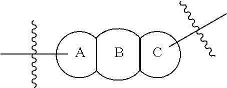
is selected from the group consisting of 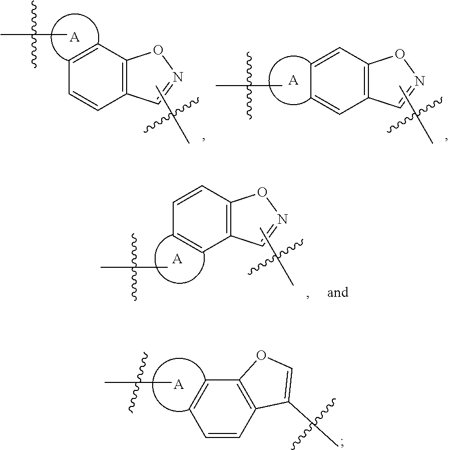
ring A is selected from the group consisting of C₅₋₁₀ cycloalkenyl, 5- to 10-membered heterocycloalkenyl, phenyl, and 5- to 6-membered heteroaryl;
L is selected from a connecting group; and
PTM, R¹ and n are as defined in any one of claim 44.

46. The compound, the stereoisomer thereof, or the pharmaceutically acceptable salt thereof according to claim 45, wherein L is selected from the group consisting of -Cy¹-LNK-Cy²-LNK-, -Cy¹-LNK-Cy²-, and -Cy¹-Cy²-LNK-, wherein Cy¹ is selected from the group consisting of a bond, C₃-12 cycloalkyl, and 4- to 12-membered heterocycloalkyl, wherein the C₃-12 cycloalkyl or 4- to 12-membered heterocycloalkyl is optionally substituted with one or more R^{a}; LNK is selected from the group consisting of a bond, C₁-12 alkylene, and C₁-12 heteroalkylene; Cy² is absent or selected from the group consisting of C₃-12 cycloalkyl and 4- to 12-membered heterocycloalkyl, wherein the cycloalkyl or heterocycloalkyl is optionally substituted with one or more R^{b}; and/or
ring A is selected from the group consisting of C₅₋₈ cycloalkenyl, 5- to 10-membered heterocycloalkenyl, phenyl, and 5- to 6-membered heteroaryl; or ring A is selected from the group consisting of C₅₋₇ cycloalkenyl, 5- to 10-membered heterocycloalkenyl, phenyl, and 5- to 6-membered heteroaryl; or ring A is selected from the group consisting of C₅₋₆ cycloalkenyl, 5- to 10-membered heterocycloalkenyl, phenyl, and 5- to 6-membered heteroaryl; or ring A is selected from the group consisting of C₅₋₆ cycloalkenyl, 5- to 9-membered heterocycloalkenyl, phenyl, pyrrolyl, pyrazolyl, furanyl, and oxazolyl; or
ring A is selected from the group consisting of C₅ cycloalkenyl, C₆ cycloalkenyl, 5-, 6-, 7-8-, or 9-membered heterocycloalkenyl, phenyl, pyrrolyl, pyrazolyl, furanyl, and oxazolyl; or
ring A is selected from the group consisting of cyclopentenyl, bicyclohexenyl, dihydropyrrolyl, tetrahydropyridinyl, tetrahydroazepinyl, azaspirooctenyl, dihydrooxazinyl, azaspirononenyl, phenyl, pyrrolyl, pyrazolyl, furanyl, and oxazolyl; or
ring A is selected from the group consisting of C₅-6 cycloalkenyl and 5- to 9-membered heterocycloalkenyl; or
ring A is selected from the group consisting of cyclopentenyl, bicyclohexenyl, dihydropyrrolyl, tetrahydropyridinyl, dihydrooxazinyl, tetrahydroazepinyl, azaspirooctenyl, and azaspirononenyl.

47. The compound, the stereoisomer thereof, or the pharmaceutically acceptable salt thereof according to claim 45, being selected from a compound of formula I′a or formula I′, a stereoisomer thereof, or a pharmaceutically acceptable salt thereof, 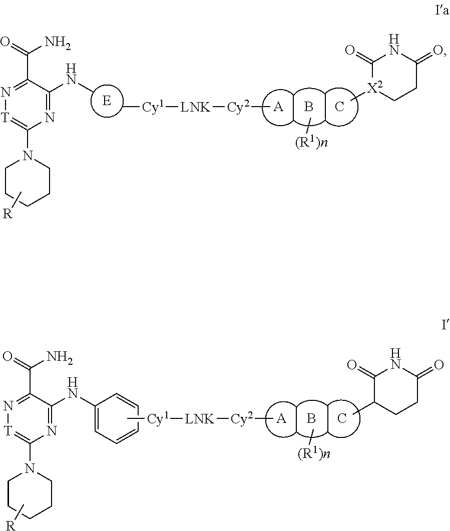
wherein,
T is selected from the group consisting of CH and N;
R is selected from the group consisting of hydrogen and 5- to 6-membered heterocycloalkyl, wherein the heterocycloalkyl is optionally substituted with ═O or C₁₋₆ alkyl;
ring E is selected from the group consisting of phenyl, benzocycloalkenyl, and benzoheterocycloalkenyl; and
X² is selected from the group consisting of CH and N.

48. The compound, the stereoisomer thereof, or the pharmaceutically acceptable salt thereof according to claim 45, wherein ring E is selected from the group consisting of phenyl, benzo C₅₋₁₂ cycloalkenyl, and benzo 5- to 12-membered heterocycloalkenyl; or
ring E is selected from the group consisting of phenyl, benzo C₅₋₆ cycloalkenyl, and benzo 5- to 11-membered heterocycloalkenyl; or
ring E is selected from the group consisting of phenyl, benzo 5-membered heterocycloalkenyl, benzo 6-membered heterocycloalkenyl, benzo 10-membered heterocycloalkenyl, and benzo 11-membered heterocycloalkenyl; and/or
ring A is absent or selected from the group consisting of C₅₋₈ cycloalkenyl, 5- to 10-membered heterocycloalkenyl, phenyl, and 5-membered heteroaryl; ring A is absent or selected from the group consisting of C₅-7 cycloalkenyl, 5- to 9-membered heterocycloalkenyl, phenyl, and 5-membered heteroaryl; ring A is absent or selected from the group consisting of C₅₋₆ cycloalkenyl, 5- to 9-membered heterocycloalkenyl, phenyl, and 5-membered heteroaryl; optionally,
ring A is absent or selected from the group consisting of C₅₋₆ cycloalkenyl, 5- to 9-membered heterocycloalkenyl, phenyl, pyrrolyl, pyrazolyl, furanyl, and oxazolyl; or
ring A is absent or selected from the group consisting of C₅ cycloalkenyl, C₆ cycloalkenyl, 5-, 6-, 7-, 8-, or 9-membered heterocycloalkenyl, phenyl, pyrrolyl, pyrazolyl, furanyl, and oxazolyl; or
ring A is absent or selected from the group consisting of cyclopentenyl, bicyclohexenyl, dihydropyrrolyl, tetrahydropyridinyl, tetrahydroazepinyl, dihydrooxazinyl, azaspirooctenyl, azaspirononenyl, phenyl, pyrrolyl, pyrazolyl, furanyl, and oxazolyl; or
ring A is selected from the group consisting of C₅₋₆ cycloalkenyl and 5- to 9-membered heterocycloalkenyl; or
ring A is selected from the group consisting of cyclopentenyl, bicyclohexenyl, dihydropyrrolyl, tetrahydropyridinyl, tetrahydroazepinyl, dihydrooxazinyl, azaspirooctenyl, and azaspirononenyl.

49. The compound, the stereoisomer thereof, or the pharmaceutically acceptable salt thereof according to claim 45, wherein the structural moiety 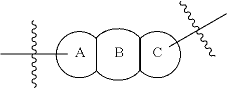
is selected from the group consisting of 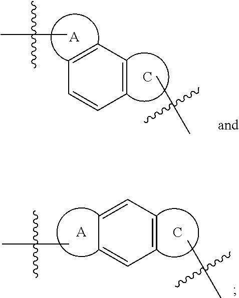
or
the structural moiety 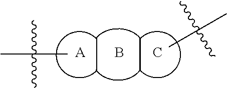
is selected from the group consisting of 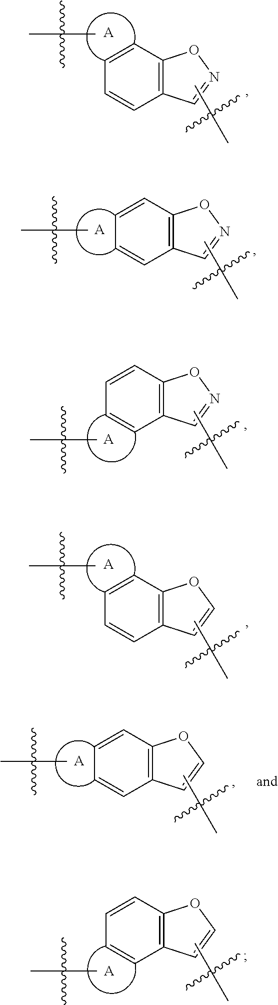
or
the structural moiety 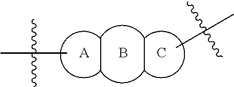
is selected from the group consisting of 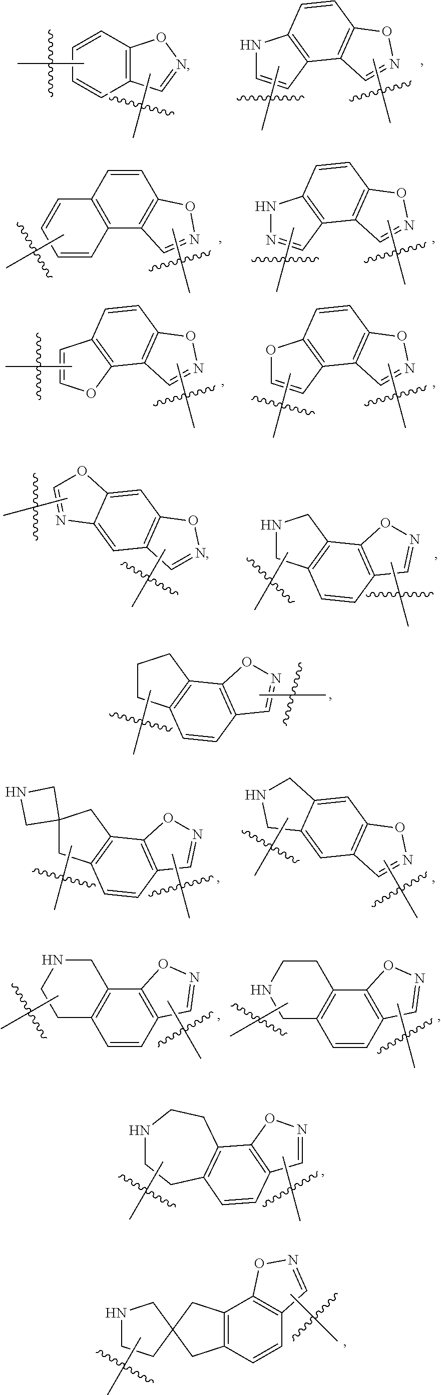 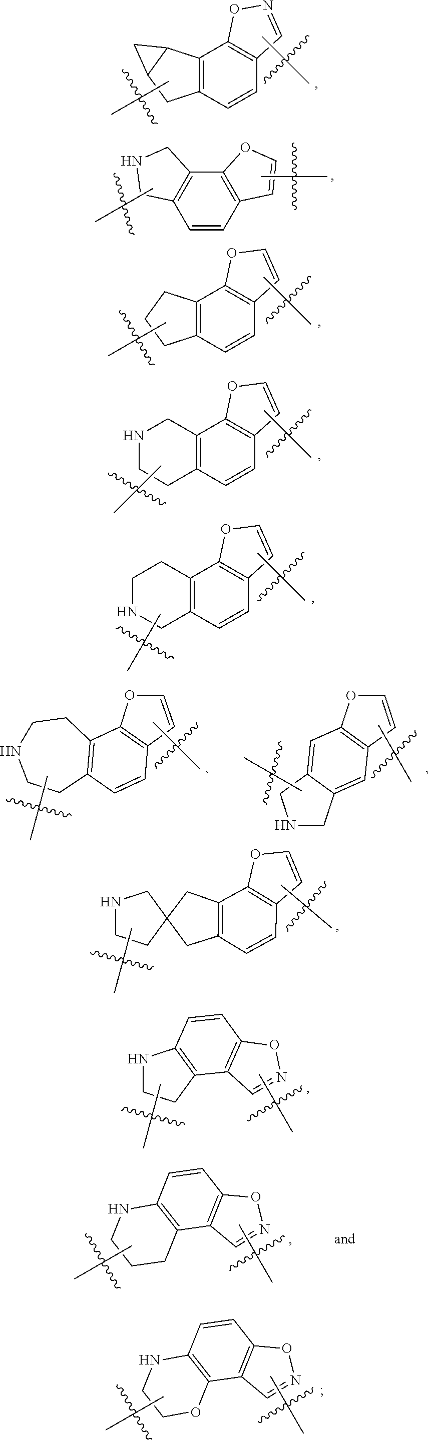
or
the structural moiety 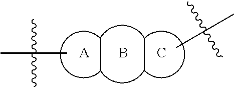
is selected from the group consisting of

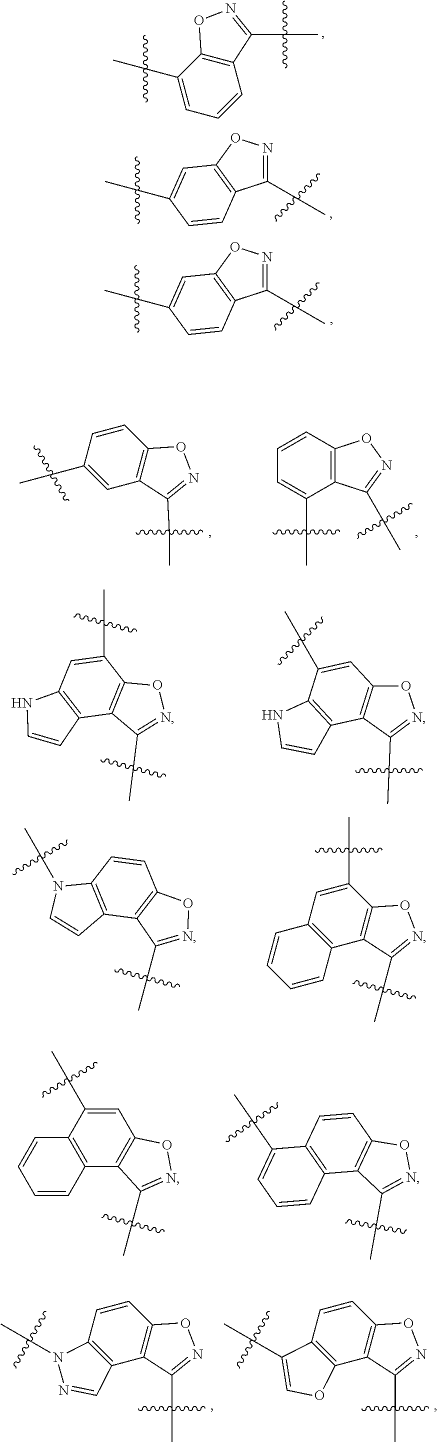

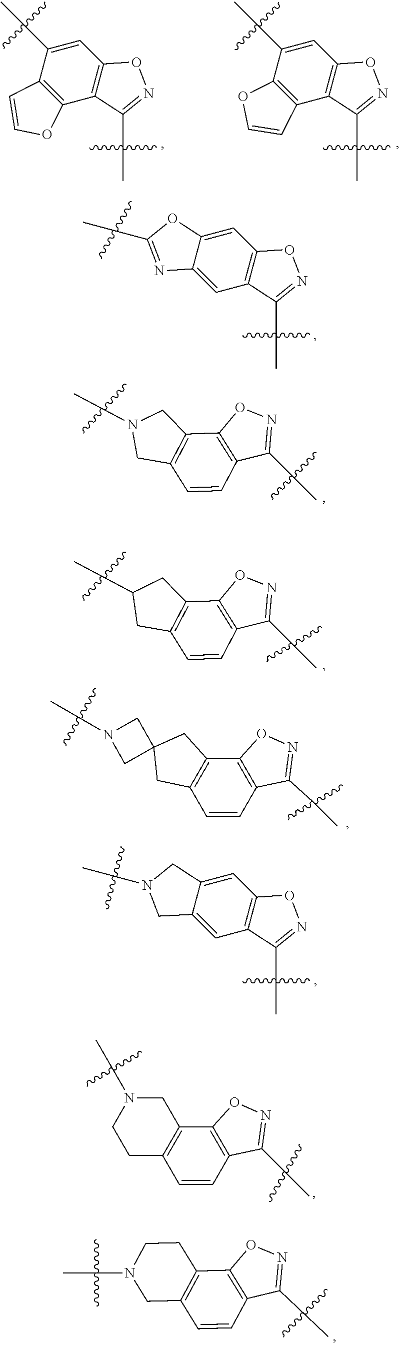

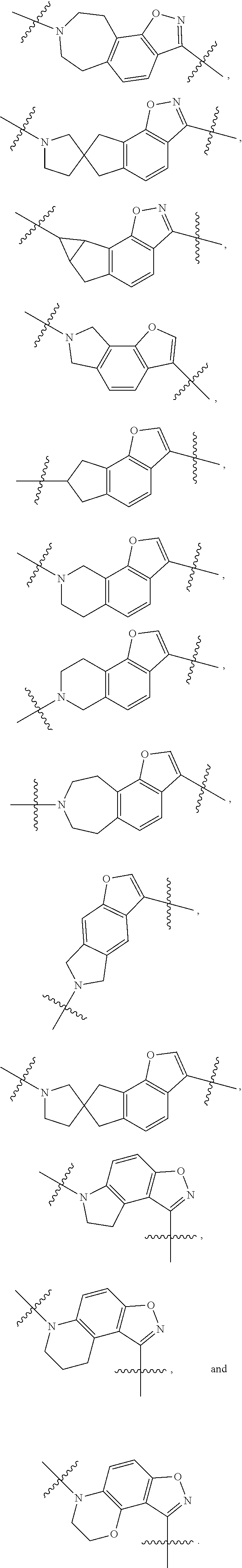

50. The compound, the stereoisomer thereof, or the pharmaceutically acceptable salt thereof according to claim 45, wherein each R¹ is independently selected from the group consisting of halogen, —OH, —NH₂, —CN, C₁₋₃ alkyl, C₁₋₃ alkoxy, and C₁₋₃ haloalkyl; or
each R¹ is independently selected from the group consisting of fluorine, chlorine, bromine, —OH, —NH₂, and —CN; or
each R¹ is independently selected from the group consisting of fluorine, chlorine, and bromine; or
each R¹ is independently selected from fluorine; and
optionally, n is selected from the group consisting of 0, 1, and 2; or, n is selected from the group consisting of 0 and 1.

51. The compound, the stereoisomer thereof, or the pharmaceutically acceptable salt thereof according to claim 45, wherein the structural moiety 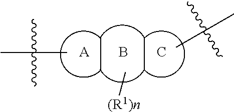
is selected from the group consisting of

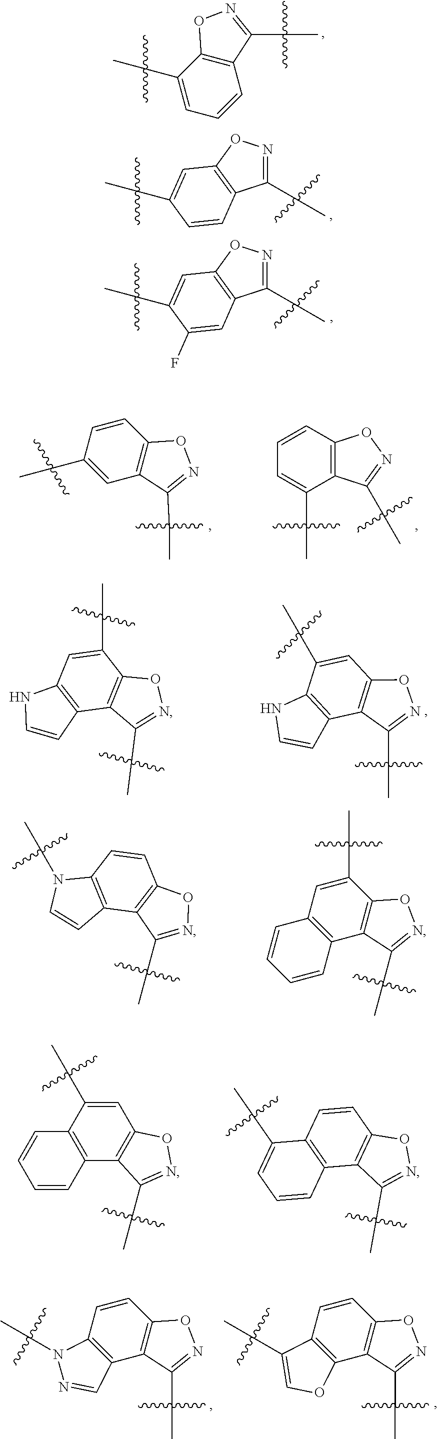

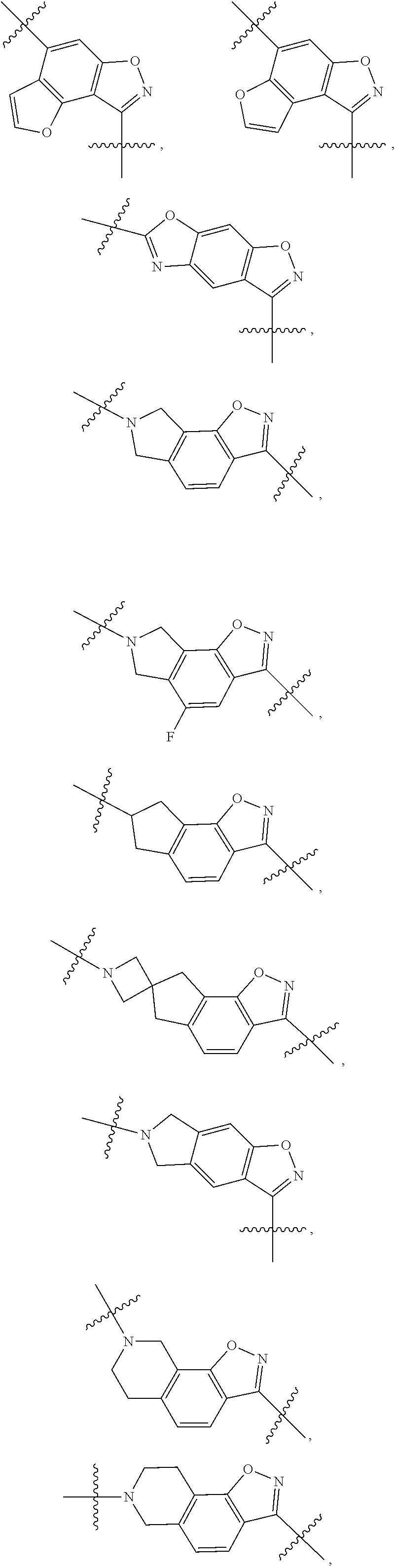

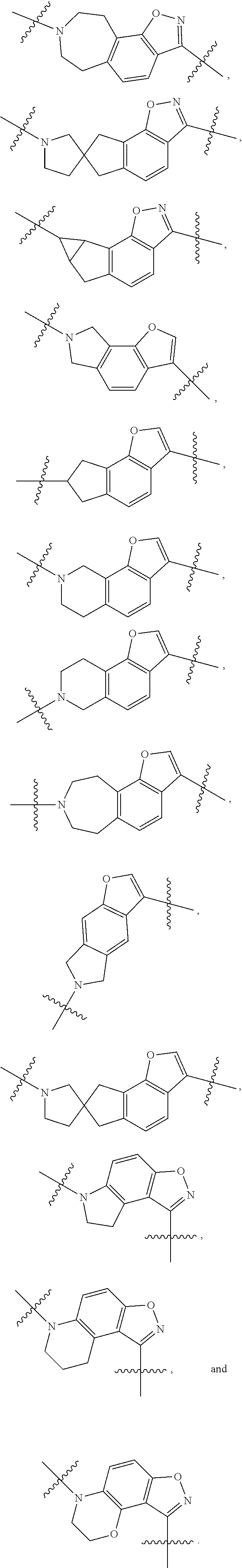

52. The compound, the stereoisomer thereof, or the pharmaceutically acceptable salt thereof according to claim 45, wherein the structural moiety -Cy¹-LNK-Cy²- is selected from the group consisting of -Cy¹-, -Cy¹-LNK, -Cy¹-Cy²-, -Cy¹-LNK-Cy²-, -Cy²-, and -LNK-Cy²-; or
the structural moiety -Cy¹-LNK-Cy²- is selected from the group consisting of -Cy¹-, -Cy¹-Cy²-, and -Cy²-; or
the structural moiety -Cy¹-LNK-Cy²- is selected from the group consisting of a bond, —CH₂—,

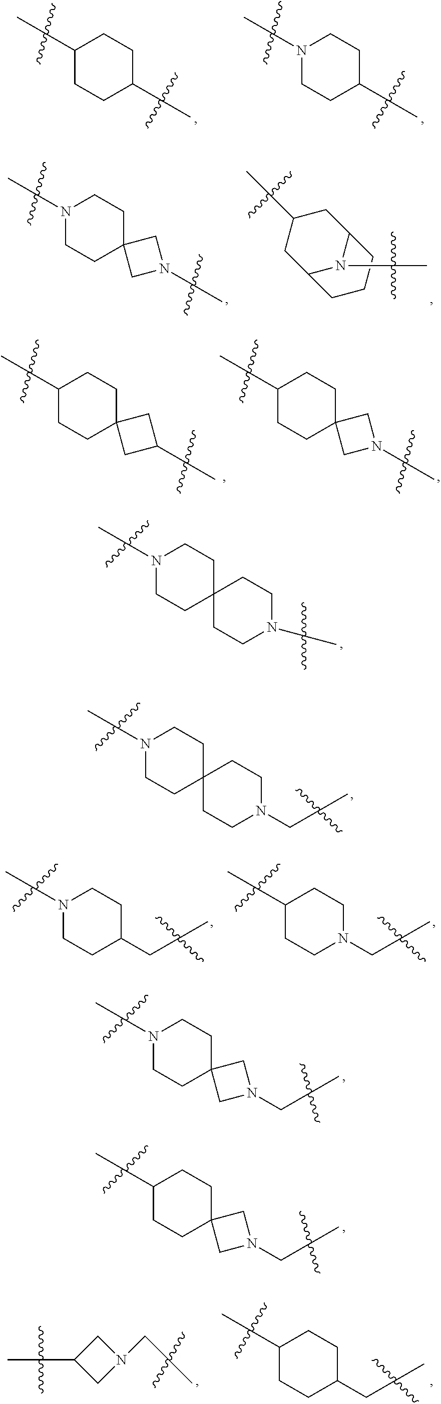

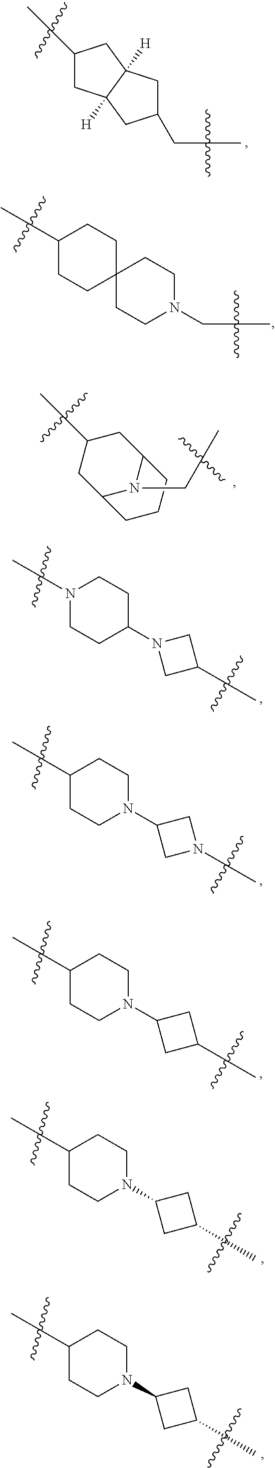

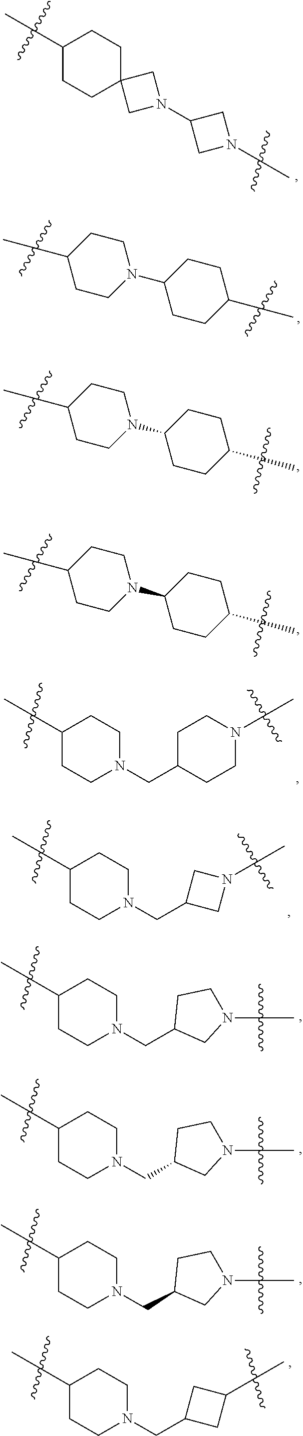

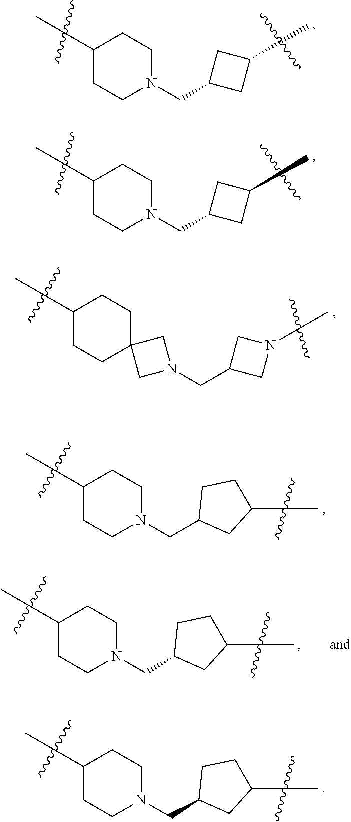

53. The compound, the stereoisomer thereof, or the pharmaceutically acceptable salt thereof according to claim 45, wherein Cy¹ is selected from the group consisting of a bond and the following groups optionally substituted with one or more R^{a}: C₄₋₁₁ cycloalkyl or 4- to 11-membered heterocycloalkyl; or
Cy¹ is selected from the group consisting of a bond and the following groups optionally substituted with one or more R^{a}: C₆₋₉ cycloalkyl or 4- to 11-membered heterocycloalkyl; or
Cy¹ is selected from the group consisting of a bond and the following groups optionally substituted with one or more R^{a}: C₆ cycloalkyl, C₉ cycloalkyl, and 4-, 5-, 6-, 7-, 8-, 9-, 10-, or 11-membered heterocycloalkyl; or
Cy¹ is selected from the group consisting of a bond and the following groups optionally substituted with one or more R^{a}: C₆ cycloalkyl, C₉ cycloalkyl, and 4-, 6-, or 8- to 11-membered heterocycloalkyl; or
Cy¹ is selected from a bond; or
Cy¹ is selected from the group consisting of C₆ cycloalkyl and C₉ cycloalkyl optionally substituted with one or more R^{a}; or
Cy¹ is selected from the group consisting of 4-, 6-, and 8- to 11-membered heterocycloalkyl optionally substituted with one or more R^{a}; or
Cy¹ is selected from the group consisting of 8-, 9-, 10-, and 11-membered heterocycloalkyl optionally substituted with one or more R^{a}; or
Cy¹ is selected from the group consisting of a bond and the following groups optionally substituted with one or more R^{a}: cyclohexyl, spirononanyl, azetidinyl, octahydrocyclopentapyrrolyl, piperidinyl, monoazaspirononanyl, diazaspirononanyl, azabicyclononanyl, monoazaspiroundecanyl, ordiazaspiroundecanyl;
optionally, LNK is selected from the group consisting of a bond, C₁₋₆ alkylene, and C₁₋₆ heteroalkylene; or
LNK is selected from the group consisting of a bond and C₁₋₄ alkylene; or
LNK is selected from the group consisting of a bond and C₁₋₃ alkylene; or
LNK is selected from the group consisting of a bond and —CH₂—;
optionally, Cy² is absent or selected from the group consisting of C₄₋₁₁ cycloalkyl and 4- to 11-membered heterocycloalkyl, wherein the cycloalkyl or heterocycloalkyl is optionally substituted with one or more R^{b}; or
Cy² is absent or selected from the group consisting of C₄₋₆ cycloalkyl and 4- to 6-membered heterocycloalkyl, wherein the cycloalkyl or heterocycloalkyl is optionally substituted with one or more R^{b}; or
Cy² is absent or selected from the group consisting of cyclobutyl, cyclopentyl, cyclohexyl, azetidinyl, pyrrolidinyl, and piperidinyl, wherein the cyclobutyl, cyclopentyl, azetidinyl, pyrrolidinyl, or piperidinyl is optionally substituted with one or more R^{b}; or
Cy² is selected from the group consisting of

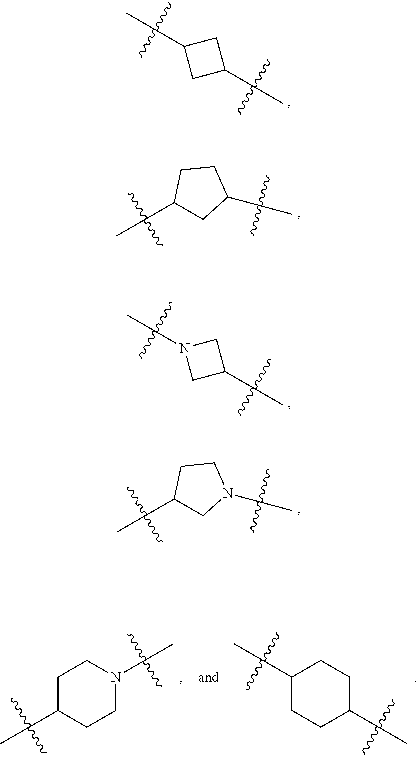 [SMILES: *C1CC(*)C1 ; *C1CCC(*)C1 ; *C1CN(*)C1 ; *C1CCN(*)C1 ; *C1CCN(*)CC1 ; *C1CCC(*)CC1]

54. The compound, the stereoisomer thereof, or the pharmaceutically acceptable salt thereof according to claim 45, wherein R^{a} and R^{b} are each independently selected from the group consisting of halogen, —OH, —NH₂, —CN, C₁₋₃ alkyl, C₁₋₃ alkoxy, C₁₋₃ haloalkyl, C₁₋₃ alkylamino, and di-C₁₋₃ alkylamino; or
R^{a} and R^{b} are each independently selected from the group consisting of halogen, —OH, —NH₂, —CN, and C₁₋₃ alkyl; or
R^{a} and R^{b} are each independently selected from the group consisting of halogen, —OH, —NH₂, and —CN.

55. The compound, the stereoisomer thereof, or the pharmaceutically acceptable salt thereof according to claim 45, wherein R is selected from the group consisting of hydrogen and 5- to 6-membered heterocycloalkyl, wherein the heterocycloalkyl is optionally substituted with ═O or C₁₋₃ alkyl; or
R is selected from the group consisting of hydrogen and 5-membered heterocycloalkyl, wherein the heterocycloalkyl is optionally substituted with ═O or C₁₋₃ alkyl; or
R is selected from the group consisting of hydrogen and imidazolinyl, wherein the imidazolinyl is optionally substituted with ═O or methyl; or
R is selected from the group consisting of hydrogen and 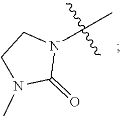 [SMILES: *N1CCN(C)C1=O]
optionally, PTM is selected from 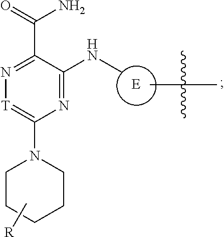
or, PTM is selected from

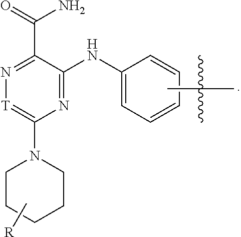

56. The compound, the stereoisomer thereof, or the pharmaceutically acceptable salt thereof according to claim 45, wherein the compound of formula I, formula II, formula I′a, or formula I′ is selected from the group consisting of compounds of formula I′-1A, formula I′-2A, formula I′-1A-1, formula I′-2A-1, formula I′-3A-1, formula I′-3A-2, formula I′-4A-1, and formula I′-4A-2, wherein: 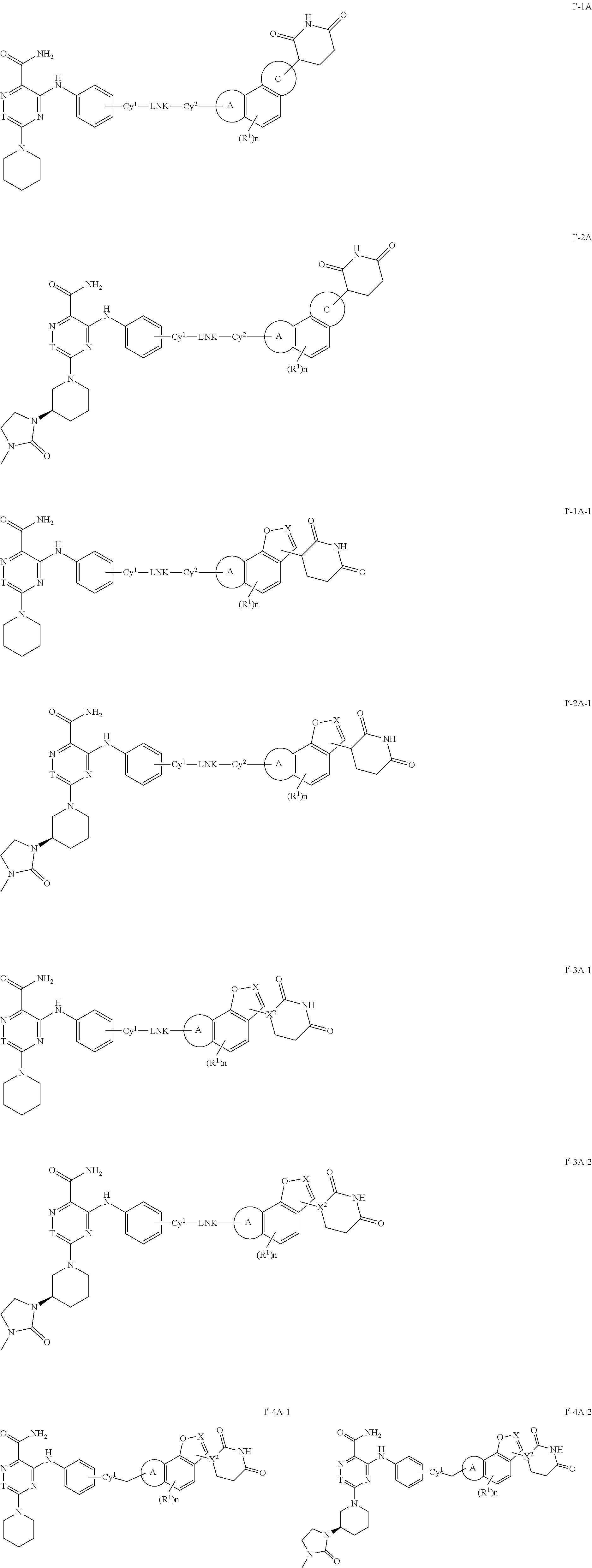
and;
X is selected from the group consisting of CH and N.

57. The compound, the stereoisomer thereof, or the pharmaceutically acceptable salt thereof according to claim 45, wherein the compound is selected from the group consisting of:

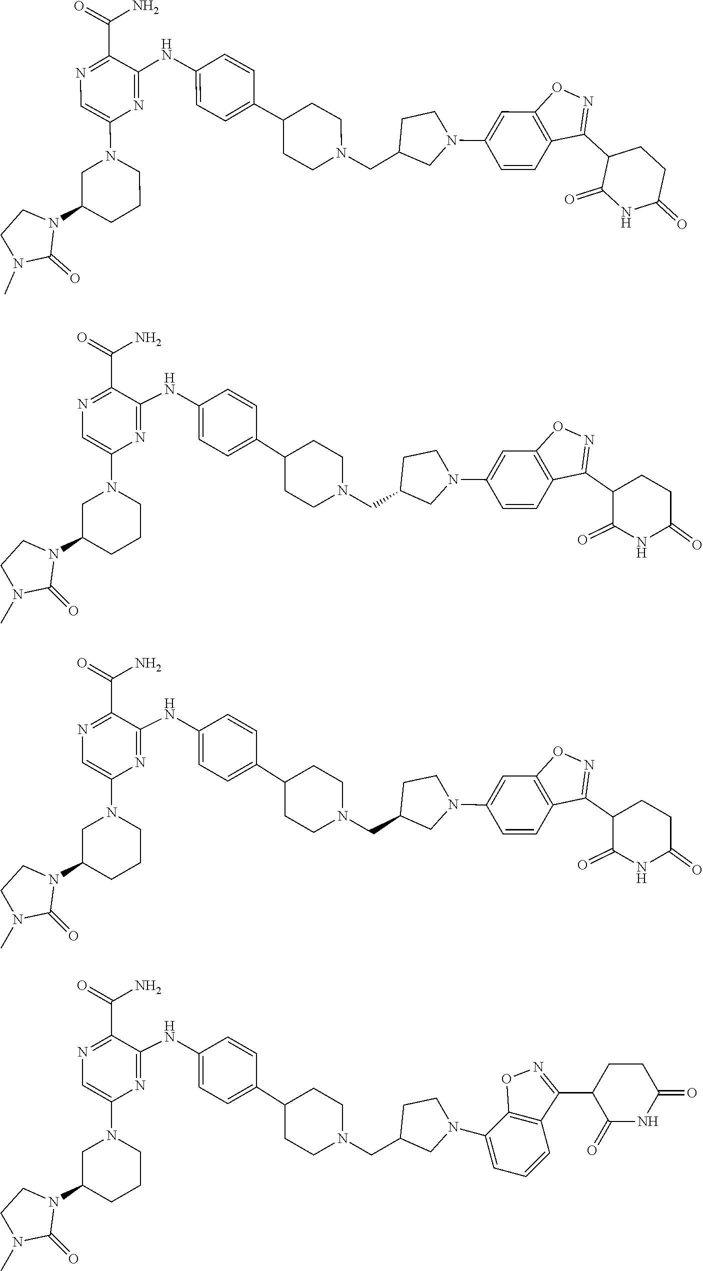 [SMILES: CN1CCN([C@@H]2CCCN(c3cnc(C(N)=O)c(Nc4ccc(C5CCN(CC6CCN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@@H]6CCN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@H]6CCN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(CC6CCN(c7cccc8c(C9CCC(=O)NC9=O)noc78)C6)CC5)cc4)n3)C2)C1=O]

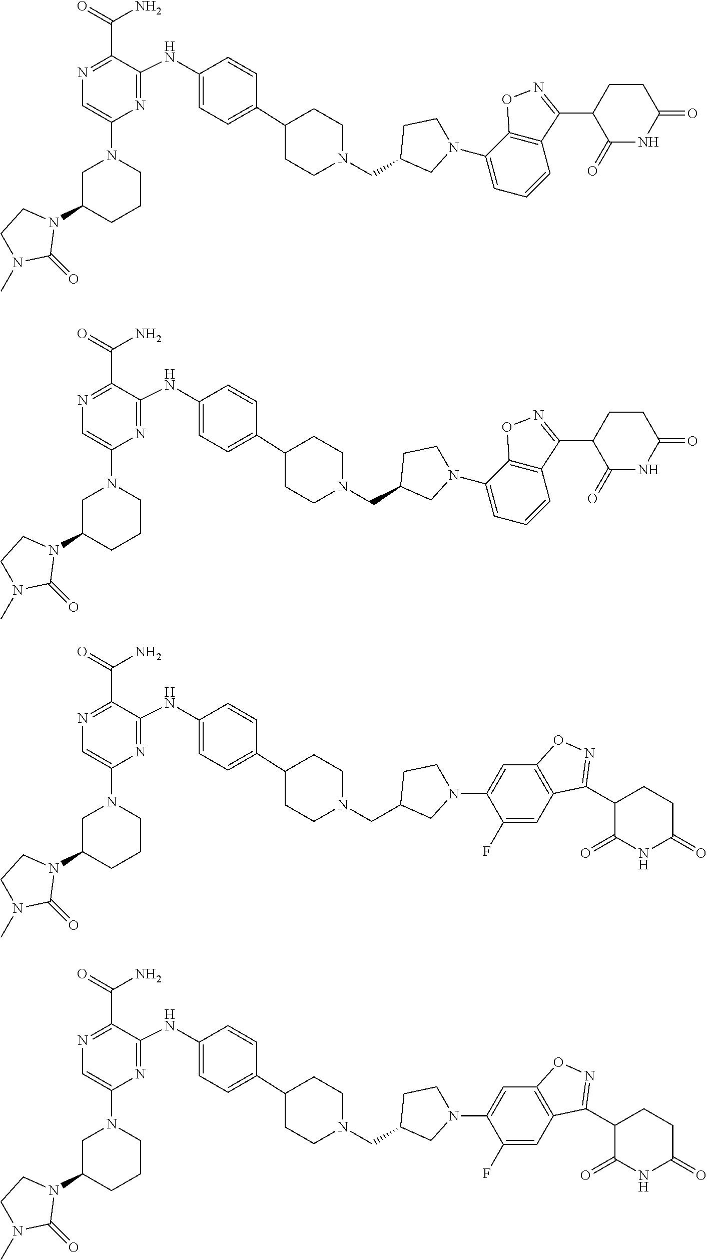

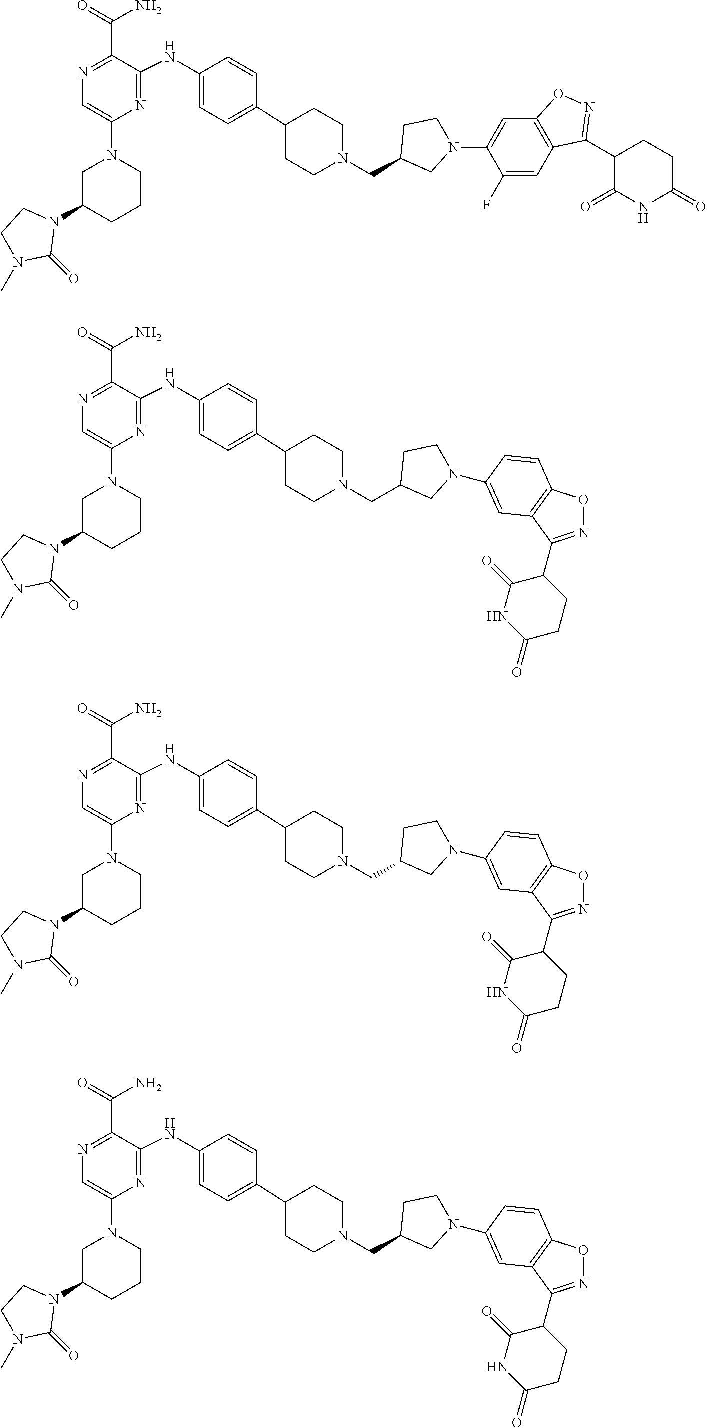 [SMILES: CN1CCN([C@@H]2CCCN(c3cnc(C(N)=O)c(Nc4ccc(C5CCN(C[C@H]6CCN(c7cc8onc(C9CCC(=O)NC9=O)c8cc7F)C6)CC5)cc4)n3)C2)C1=O ; CN1CCN([C@@H]2CCCN(c3cnc(C(N)=O)c(Nc4ccc(C5CCN(CC6CCN(c7ccc8onc(C9CCC(=O)NC9=O)c8c7)C6)CC5)cc4)n3)C2)C1=O ; CN1CCN([C@@H]2CCCN(c3cnc(C(N)=O)c(Nc4ccc(C5CCN(C[C@@H]6CCN(c7ccc8onc(C9CCC(=O)NC9=O)c8c7)C6)CC5)cc4)n3)C2)C1=O ; CN1CCN([C@@H]2CCCN(c3cnc(C(N)=O)c(Nc4ccc(C5CCN(C[C@H]6CCN(c7ccc8onc(C9CCC(=O)NC9=O)c8c7)C6)CC5)cc4)n3)C2)C1=O]

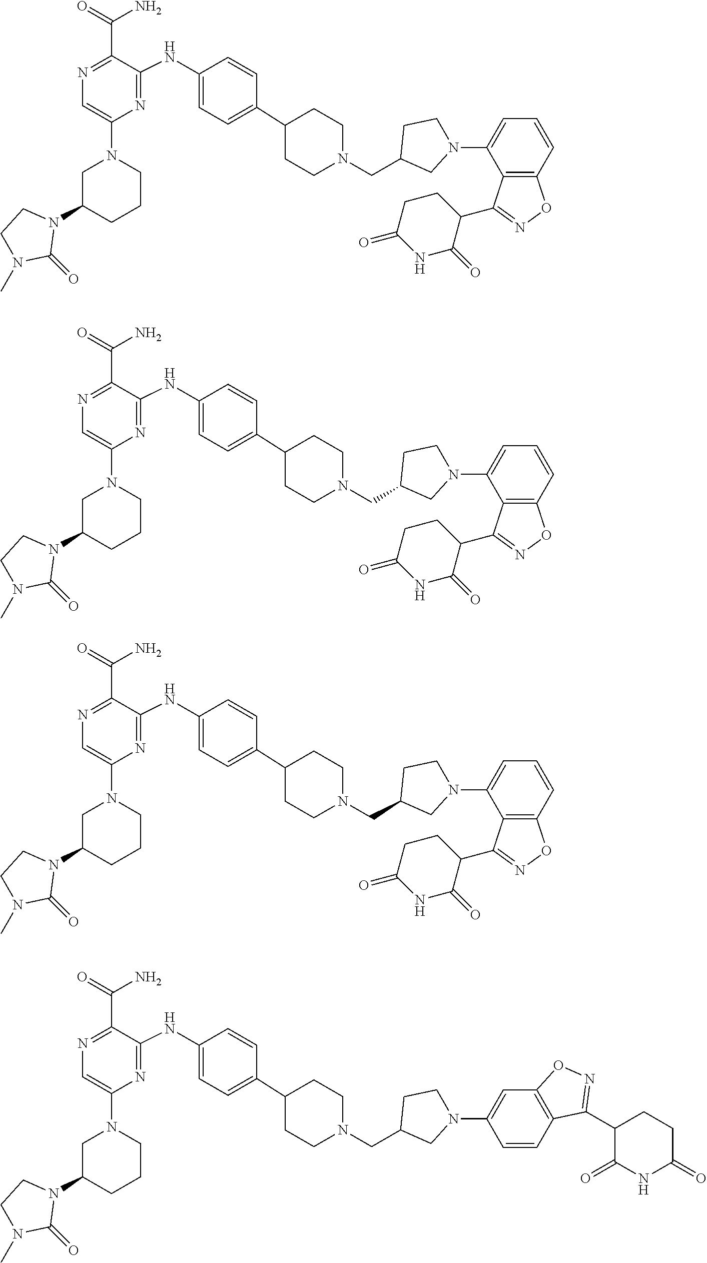

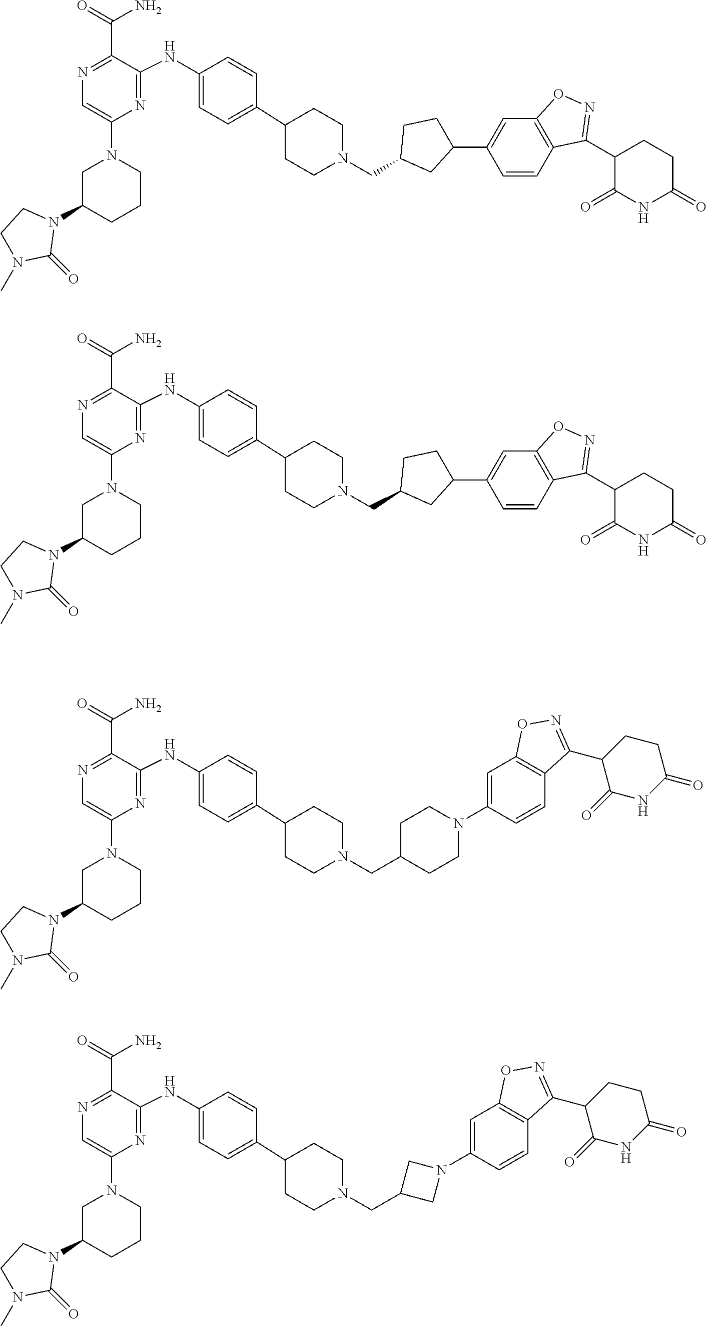 [SMILES: CN1CCN([C@@H]2CCCN(c3cnc(C(N)=O)c(Nc4ccc(C5CCN(C[C@@H]6CCC(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(C[C@H]6CCC(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCN(CC6CCN(c7ccc8c(C9CCC(=O)NC9=O)noc8c7)CC6)CC5)cc4)n3)C2)C1=O ; CN1CCN([C@@H]2CCCN(c3cnc(C(N)=O)c(Nc4ccc(C5CCN(CC6CN(c7ccc8c(C9CCC(=O)NC9=O)noc8c7)C6)CC5)cc4)n3)C2)C1=O]

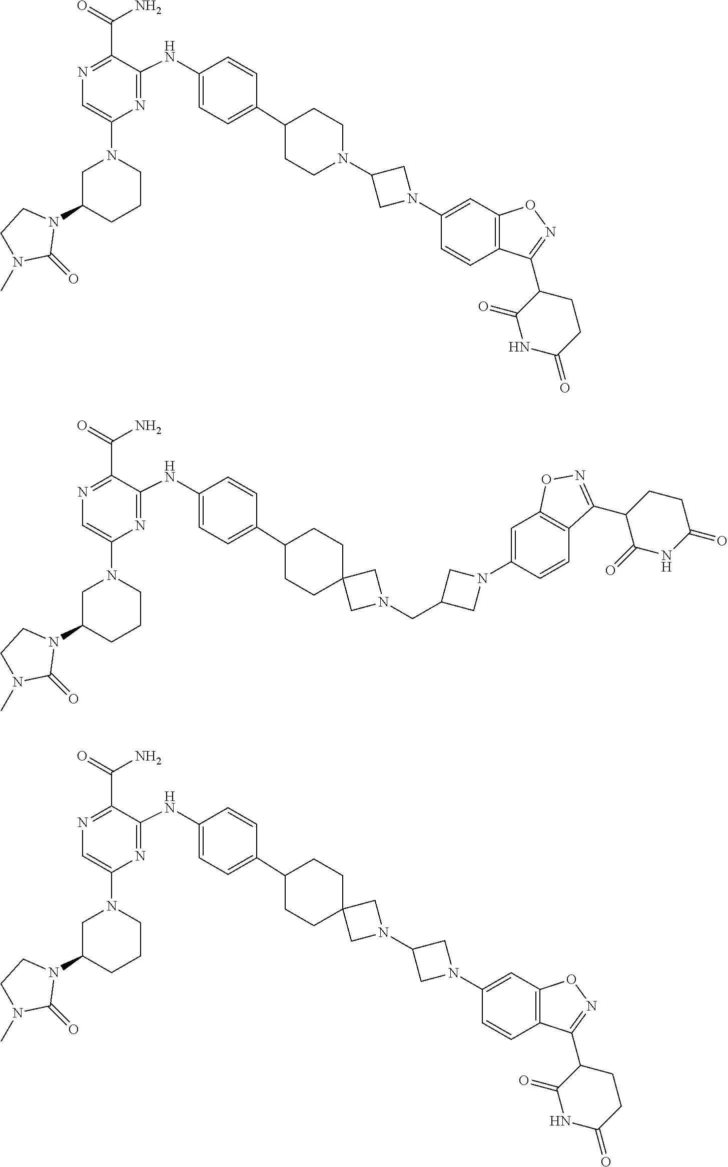 [SMILES: CN1CCN([C@@H]2CCCN(c3cnc(C(N)=O)c(Nc4ccc(C5CCN(C6CN(c7ccc8c(C9CCC(=O)NC9=O)noc8c7)C6)CC5)cc4)n3)C2)C1=O ; CN1CCN([C@@H]2CCCN(c3cnc(C(N)=O)c(Nc4ccc(C5CCC6(CC5)CN(CC5CN(c7ccc8c(C9CCC(=O)NC9=O)noc8c7)C5)C6)cc4)n3)C2)C1=O ; CN1CCN([C@@H]2CCCN(c3cnc(C(N)=O)c(Nc4ccc(C5CCC6(CC5)CN(C5CN(c7ccc8c(C9CCC(=O)NC9=O)noc8c7)C5)C6)cc4)n3)C2)C1=O]

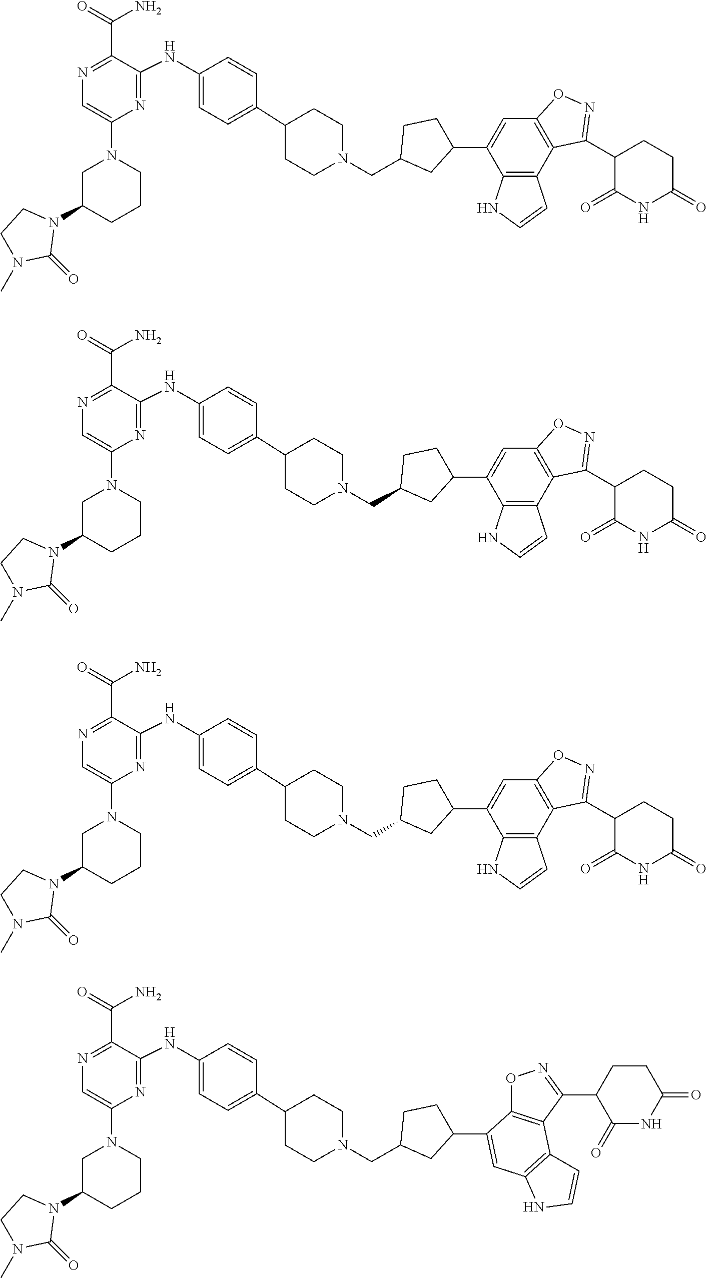 [SMILES: CN1CCN([C@@H]2CCCN(c3cnc(C(N)=O)c(Nc4ccc(C5CCN(CC6CCC(c7cc8onc(C9CCC(=O)NC9=O)c8c8cc[nH]c78)C6)CC5)cc4)n3)C2)C1=O ; CN1CCN([C@@H]2CCCN(c3cnc(C(N)=O)c(Nc4ccc(C5CCN(C[C@H]6CCC(c7cc8onc(C9CCC(=O)NC9=O)c8c8cc[nH]c78)C6)CC5)cc4)n3)C2)C1=O ; CN1CCN([C@@H]2CCCN(c3cnc(C(N)=O)c(Nc4ccc(C5CCN(C[C@@H]6CCC(c7cc8onc(C9CCC(=O)NC9=O)c8c8cc[nH]c78)C6)CC5)cc4)n3)C2)C1=O ; CN1CCN([C@@H]2CCCN(c3cnc(C(N)=O)c(Nc4ccc(C5CCN(CC6CCC(c7cc8[nH]ccc8c8c(C9CCC(=O)NC9=O)noc78)C6)CC5)cc4)n3)C2)C1=O]

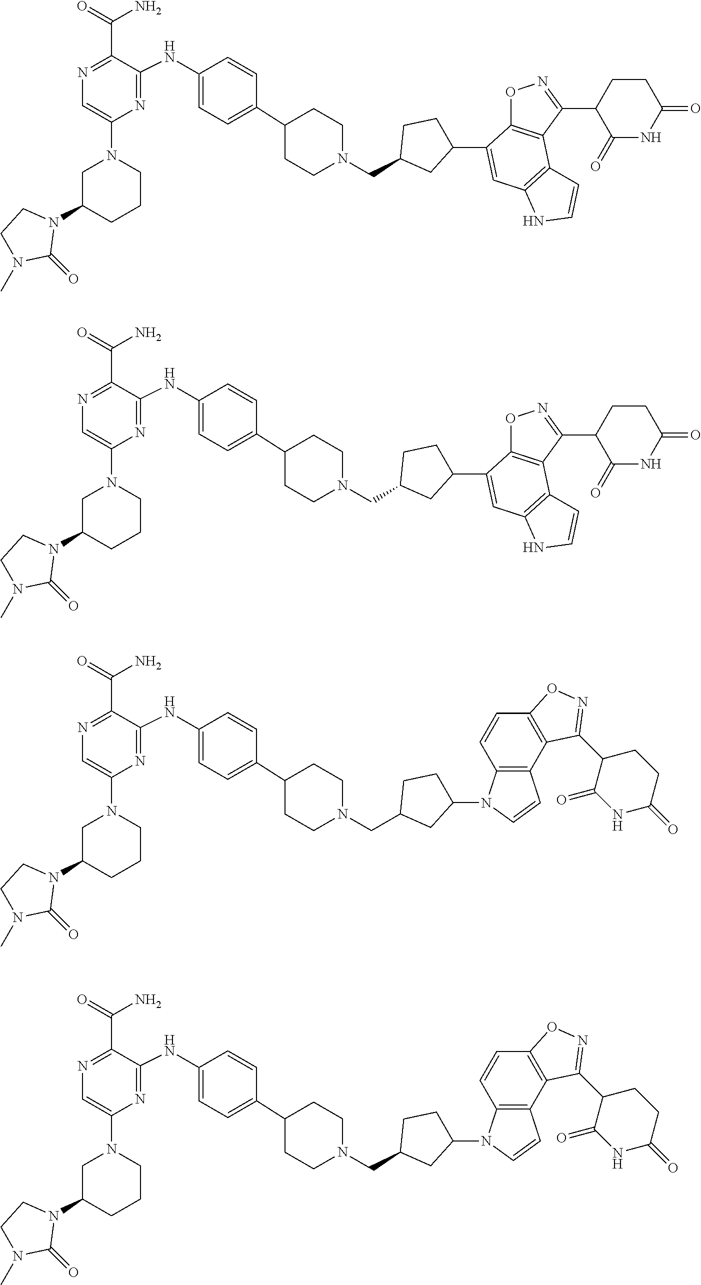 [SMILES: CN1CCN([C@@H]2CCCN(c3cnc(C(N)=O)c(Nc4ccc(C5CCN(C[C@H]6CCC(c7cc8[nH]ccc8c8c(C9CCC(=O)NC9=O)noc78)C6)CC5)cc4)n3)C2)C1=O ; CN1CCN([C@@H]2CCCN(c3cnc(C(N)=O)c(Nc4ccc(C5CCN(C[C@@H]6CCC(c7cc8[nH]ccc8c8c(C9CCC(=O)NC9=O)noc78)C6)CC5)cc4)n3)C2)C1=O ; CN1CCN([C@@H]2CCCN(c3cnc(C(N)=O)c(Nc4ccc(C5CCN(CC6CCC(n7ccc8c9c(C%10CCC(=O)NC%10=O)noc9ccc87)C6)CC5)cc4)n3)C2)C1=O ; CN1CCN([C@@H]2CCCN(c3cnc(C(N)=O)c(Nc4ccc(C5CCN(C[C@H]6CCC(n7ccc8c9c(C%10CCC(=O)NC%10=O)noc9ccc87)C6)CC5)cc4)n3)C2)C1=O]

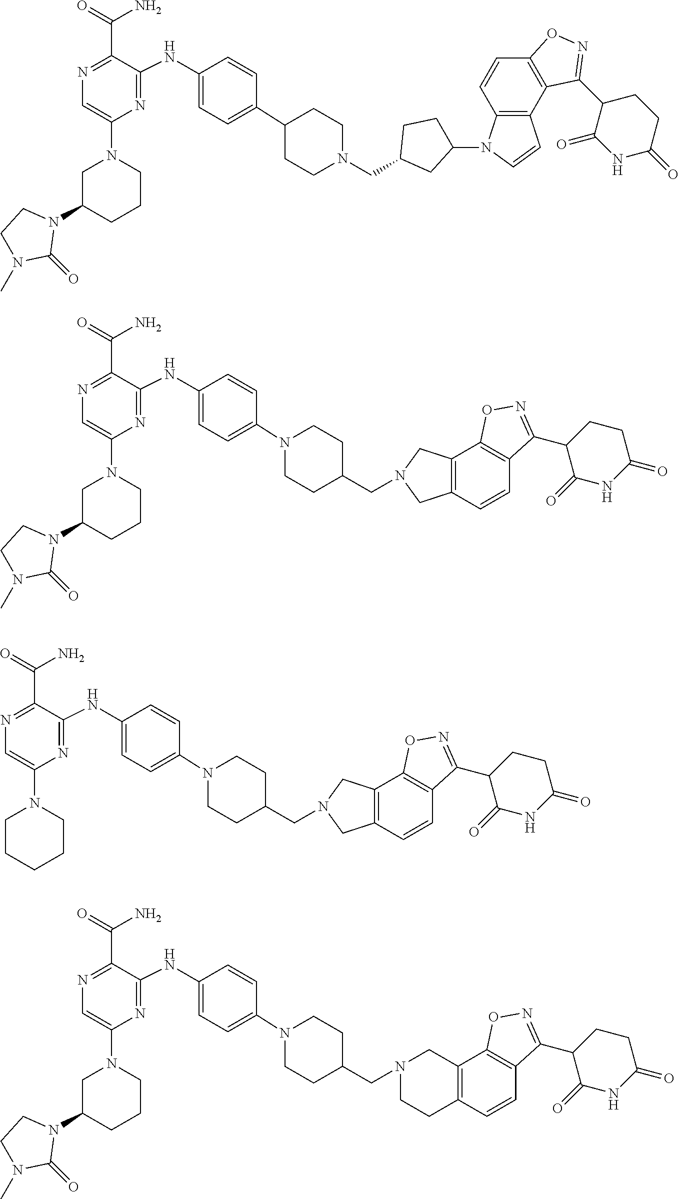

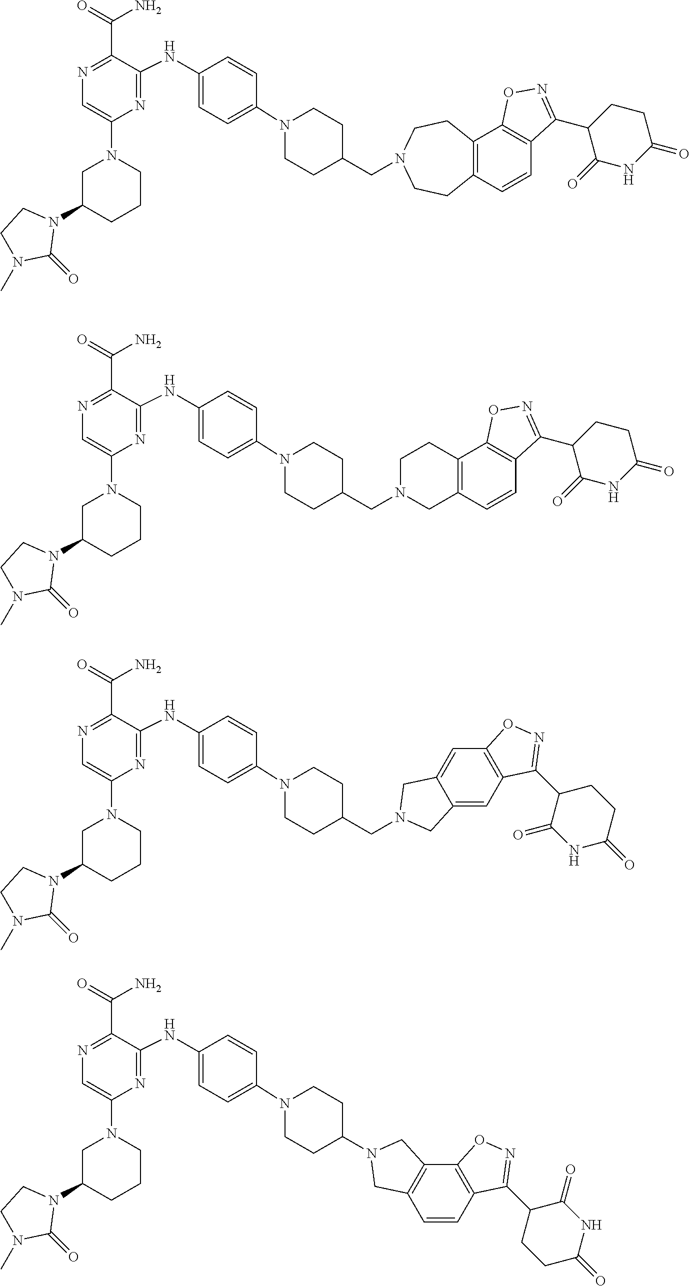

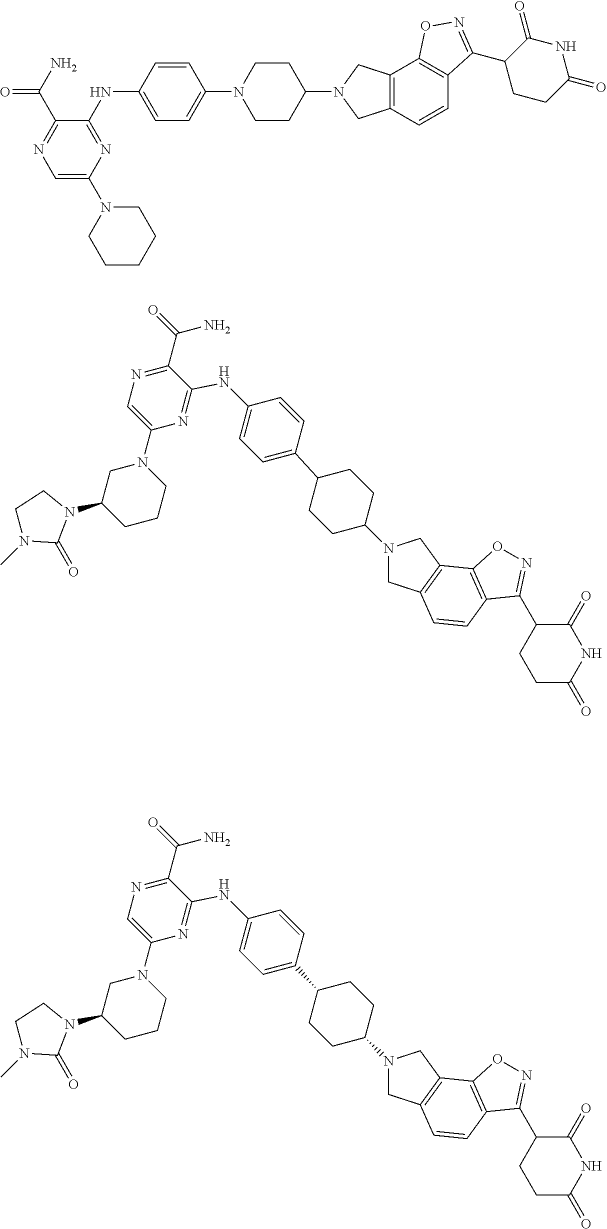 [SMILES: NC(=O)c1ncc(N2CCCCC2)nc1Nc1ccc(N2CCC(N3Cc4ccc5c(C6CCC(=O)NC6=O)noc5c4C3)CC2)cc1 ; CN1CCN([C@@H]2CCCN(c3cnc(C(N)=O)c(Nc4ccc(C5CCC(N6Cc7ccc8c(C9CCC(=O)NC9=O)noc8c7C6)CC5)cc4)n3)C2)C1=O ; CN1CCN([C@@H]2CCCN(c3cnc(C(N)=O)c(Nc4ccc([C@H]5CC[C@@H](N6Cc7ccc8c(C9CCC(=O)NC9=O)noc8c7C6)CC5)cc4)n3)C2)C1=O]

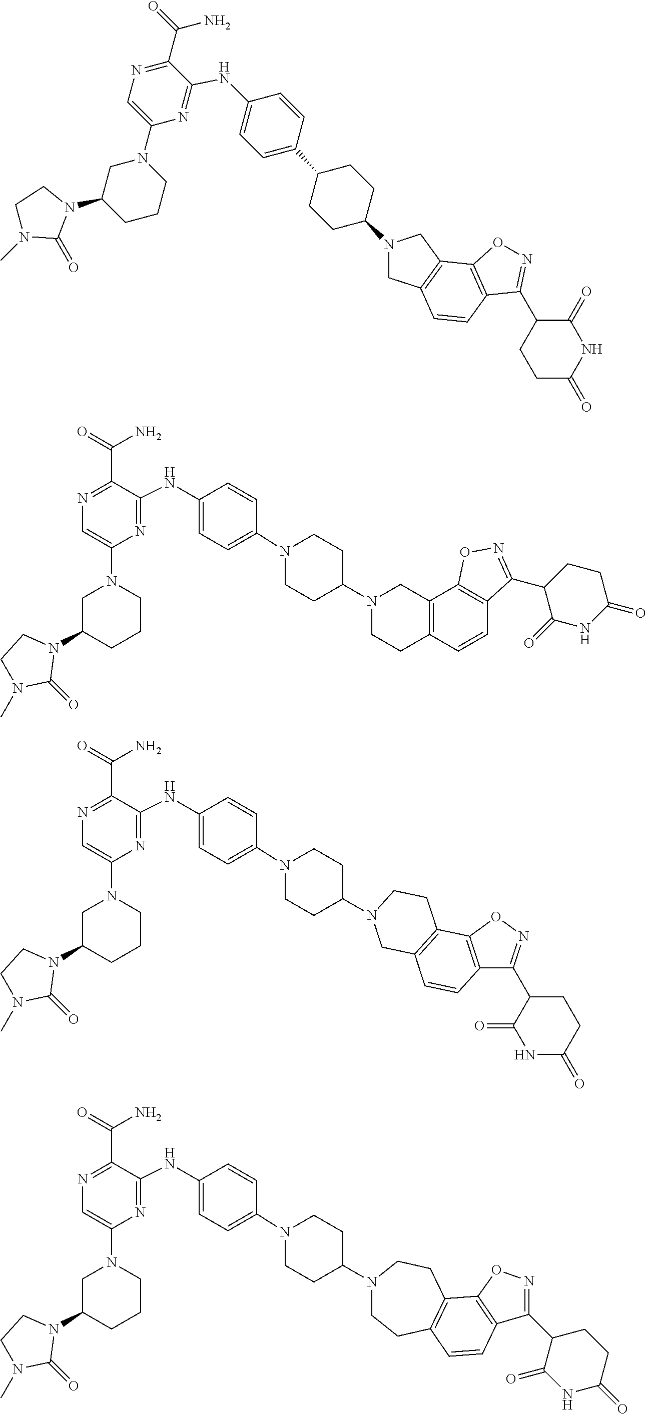

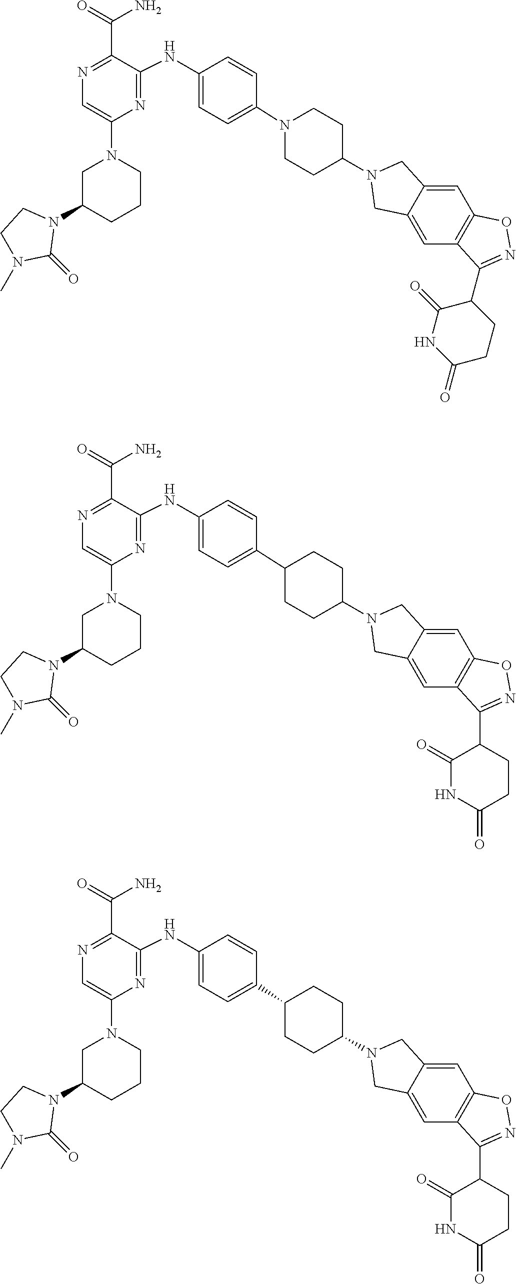 [SMILES: CN1CCN([C@@H]2CCCN(c3cnc(C(N)=O)c(Nc4ccc(N5CCC(N6Cc7cc8onc(C9CCC(=O)NC9=O)c8cc7C6)CC5)cc4)n3)C2)C1=O ; CN1CCN([C@@H]2CCCN(c3cnc(C(N)=O)c(Nc4ccc(C5CCC(N6Cc7cc8onc(C9CCC(=O)NC9=O)c8cc7C6)CC5)cc4)n3)C2)C1=O ; CN1CCN([C@@H]2CCCN(c3cnc(C(N)=O)c(Nc4ccc([C@H]5CC[C@@H](N6Cc7cc8onc(C9CCC(=O)NC9=O)c8cc7C6)CC5)cc4)n3)C2)C1=O]

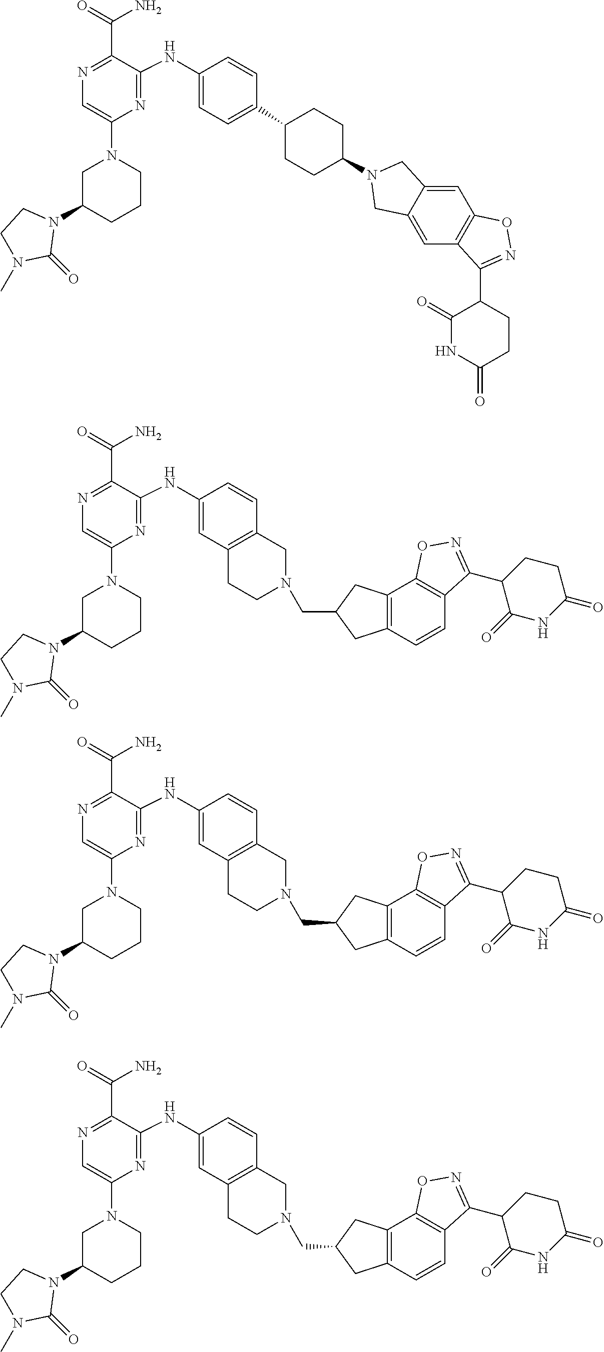

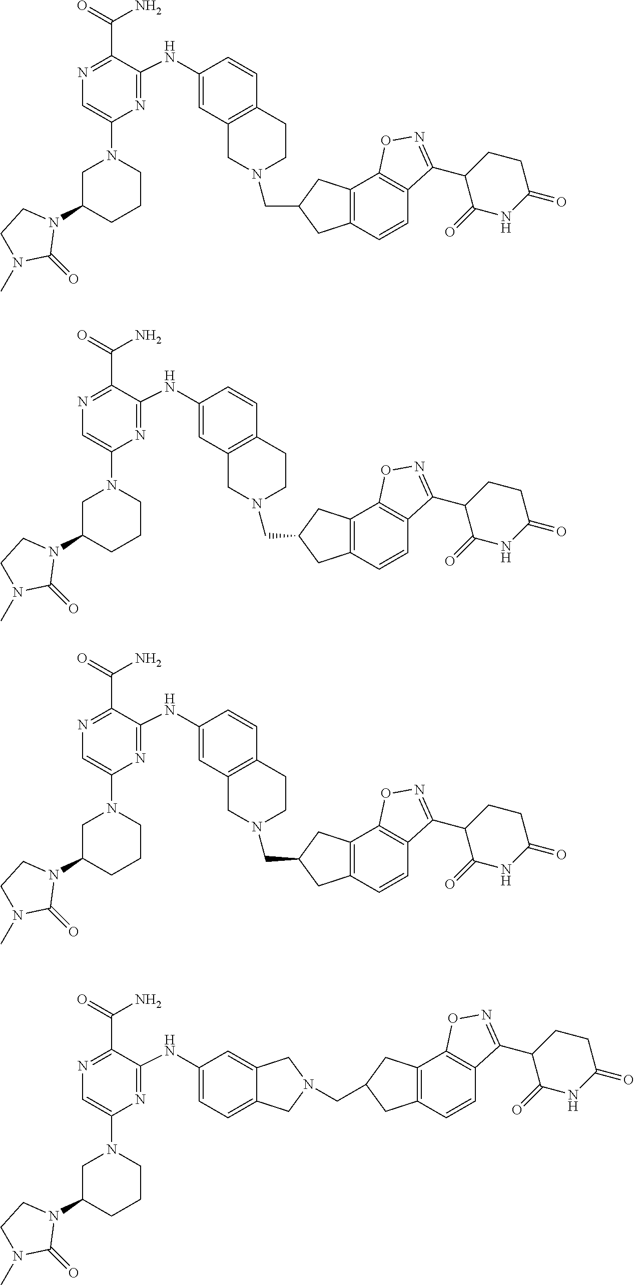 [SMILES: CN1CCN([C@@H]2CCCN(c3cnc(C(N)=O)c(Nc4ccc5c(c4)CN(CC4Cc6ccc7c(C8CCC(=O)NC8=O)noc7c6C4)CC5)n3)C2)C1=O ; CN1CCN([C@@H]2CCCN(c3cnc(C(N)=O)c(Nc4ccc5c(c4)CN(C[C@H]4Cc6ccc7c(C8CCC(=O)NC8=O)noc7c6C4)CC5)n3)C2)C1=O ; CN1CCN([C@@H]2CCCN(c3cnc(C(N)=O)c(Nc4ccc5c(c4)CN(C[C@@H]4Cc6ccc7c(C8CCC(=O)NC8=O)noc7c6C4)CC5)n3)C2)C1=O ; CN1CCN([C@@H]2CCCN(c3cnc(C(N)=O)c(Nc4ccc5c(c4)CN(CC4Cc6ccc7c(C8CCC(=O)NC8=O)noc7c6C4)C5)n3)C2)C1=O]

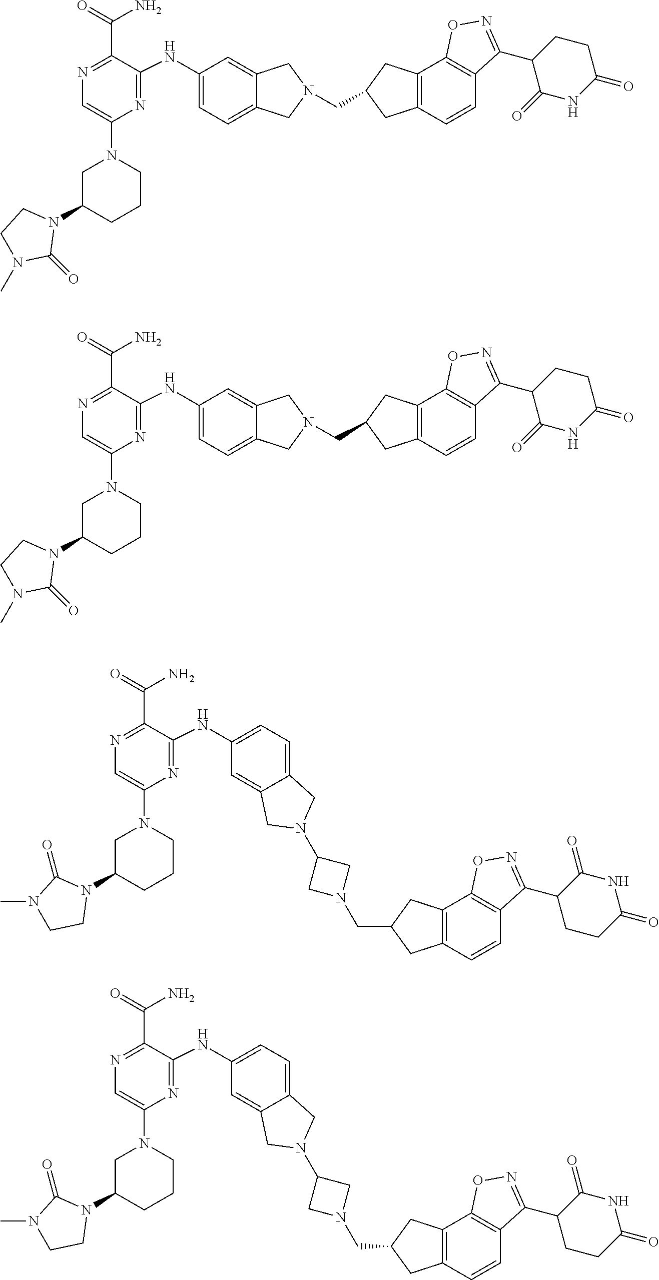 [SMILES: CN1CCN([C@@H]2CCCN(c3cnc(C(N)=O)c(Nc4ccc5c(c4)CN(C[C@H]4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CN(C[C@@H]4Cc6ccc7c(C8CCC(=O)NC8=O)noc7c6C4)C5)n3)C2)C1=O ; CN1CCN([C@@H]2CCCN(c3cnc(C(N)=O)c(Nc4ccc5c(c4)CN(C4CN(CC6Cc7ccc8c(C9CCC(=O)NC9=O)noc8c7C6)C4)C5)n3)C2)C1=O ; CN1CCN([C@@H]2CCCN(c3cnc(C(N)=O)c(Nc4ccc5c(c4)CN(C4CN(C[C@H]6Cc7ccc8c(C9CCC(=O)NC9=O)noc8c7C6)C4)C5)n3)C2)C1=O]

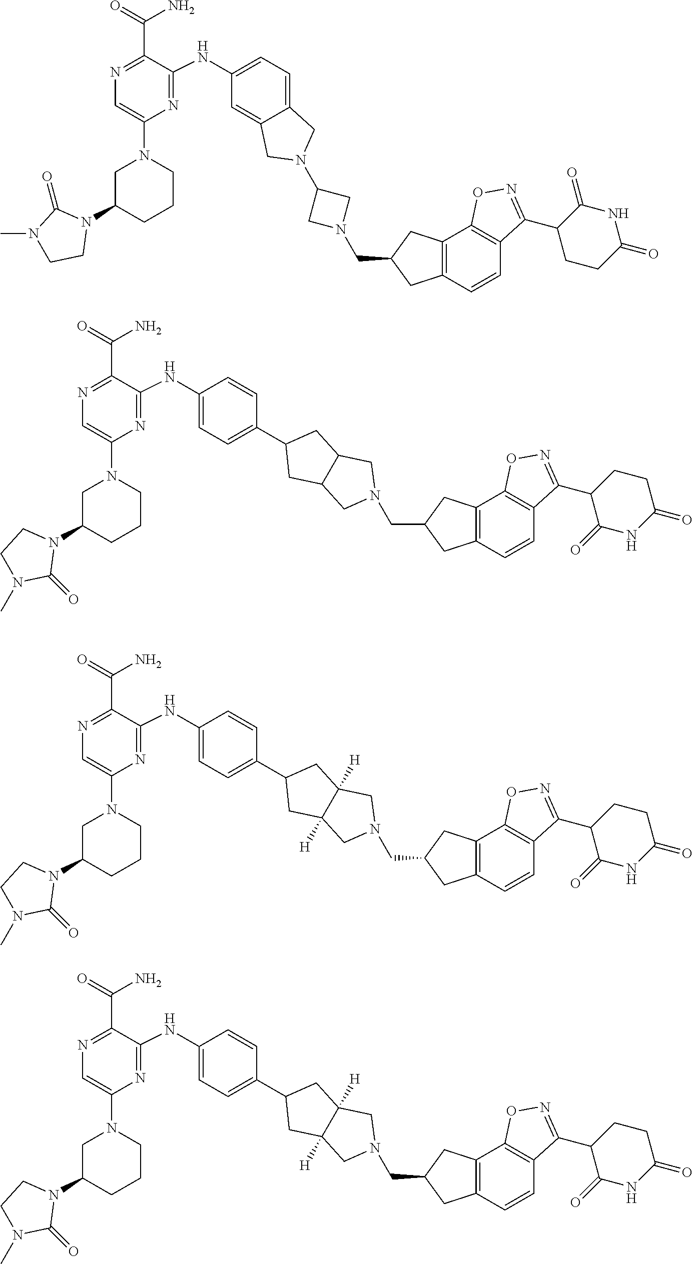

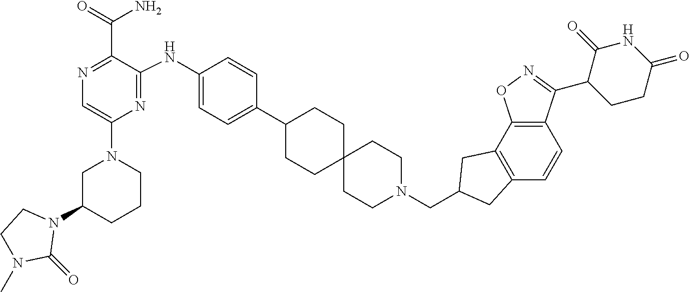 [SMILES: CN1CCN([C@@H]2CCCN(c3cnc(C(N)=O)c(Nc4ccc(C5CCC6(CC5)CCN(CC5Cc7ccc8c(C9CCC(=O)NC9=O)noc8c7C5)CC6)cc4)n3)C2)C1=O]

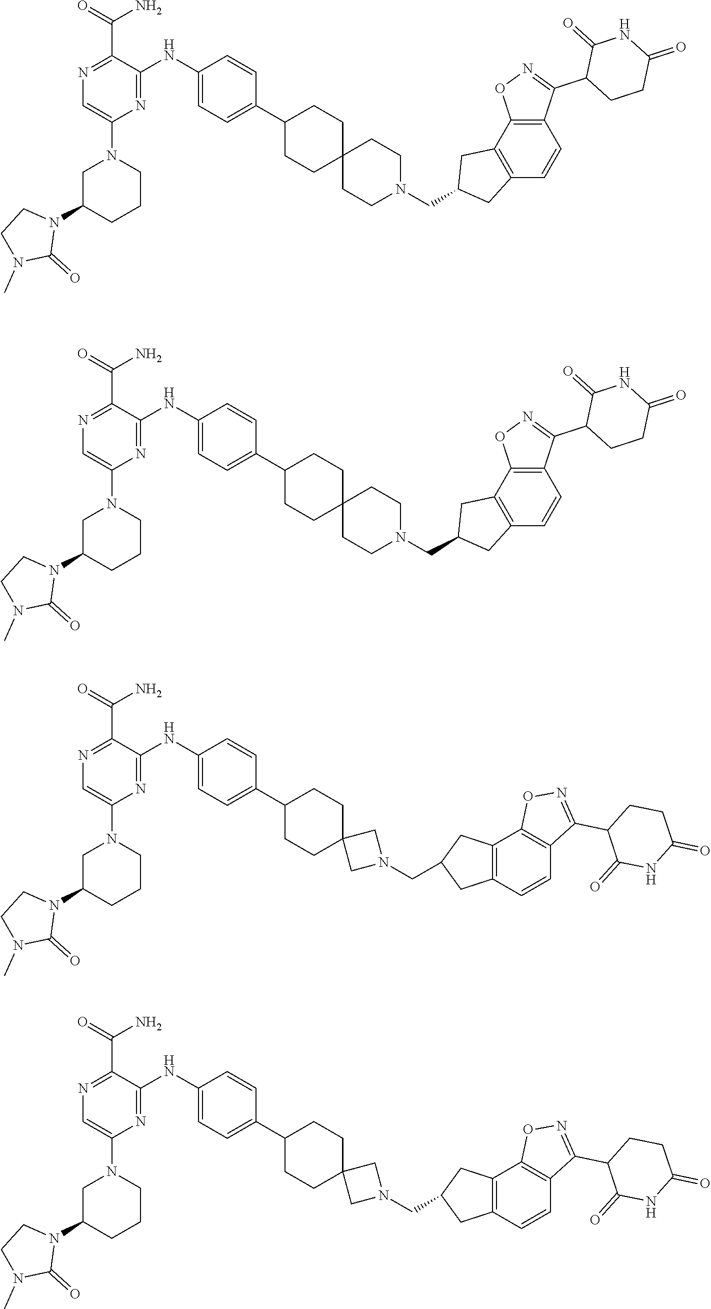 [SMILES: CN1CCN([C@@H]2CCCN(c3cnc(C(N)=O)c(Nc4ccc(C5CCC6(CC5)CCN(C[C@H]5Cc7ccc8c(C9CCC(=O)NC9=O)noc8c7C5)CC6)cc4)n3)C2)C1=O ; CN1CCN([C@@H]2CCCN(c3cnc(C(N)=O)c(Nc4ccc(C5CCC6(CC5)CCN(C[C@@H]5Cc7ccc8c(C9CCC(=O)NC9=O)noc8c7C5)CC6)cc4)n3)C2)C1=O ; CN1CCN([C@@H]2CCCN(c3cnc(C(N)=O)c(Nc4ccc(C5CCC6(CC5)CN(CC5Cc7ccc8c(C9CCC(=O)NC9=O)noc8c7C5)C6)cc4)n3)C2)C1=O ; CN1CCN([C@@H]2CCCN(c3cnc(C(N)=O)c(Nc4ccc(C5CCC6(CC5)CN(C[C@H]5Cc7ccc8c(C9CCC(=O)NC9=O)noc8c7C5)C6)cc4)n3)C2)C1=O]

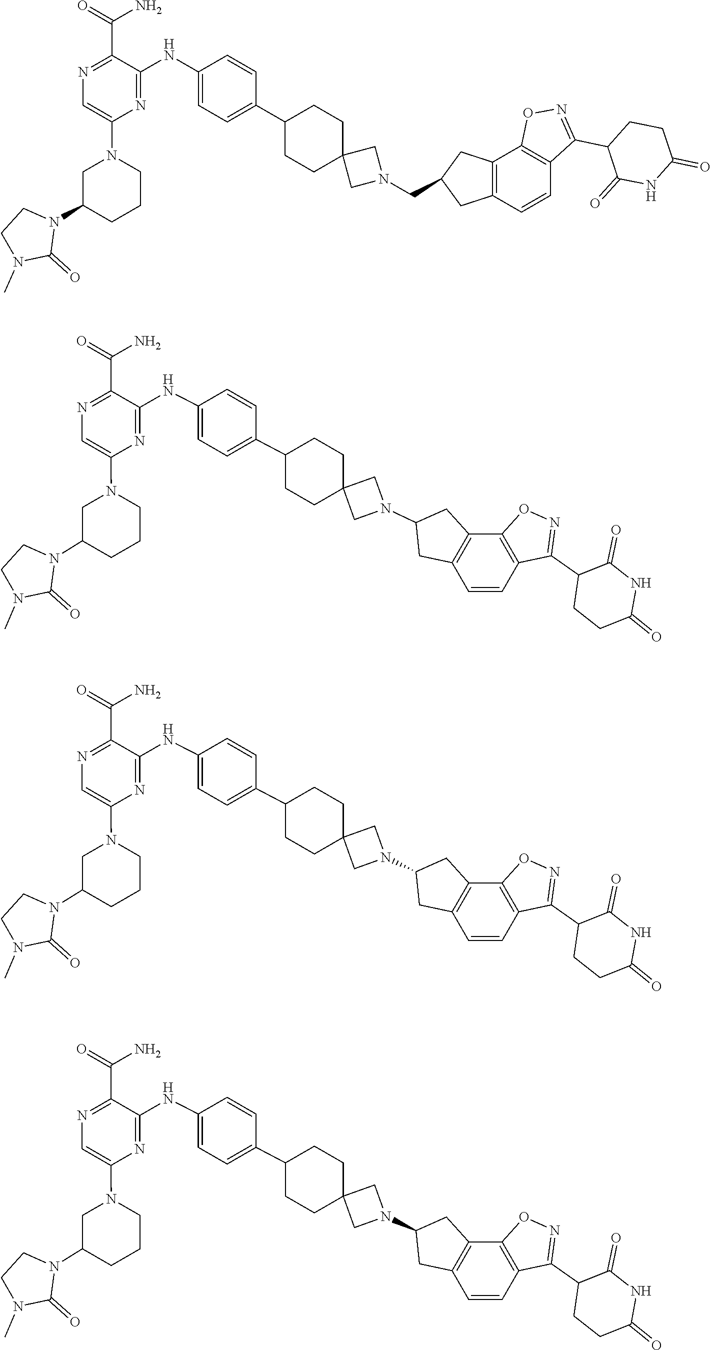 [SMILES: CN1CCN([C@@H]2CCCN(c3cnc(C(N)=O)c(Nc4ccc(C5CCC6(CC5)CN(C[C@@H]5Cc7ccc8c(C9CCC(=O)NC9=O)noc8c7C5)C6)cc4)n3)C2)C1=O ; CN1CCN(C2CCCN(c3cnc(C(N)=O)c(Nc4ccc(C5CCC6(CC5)CN(C5Cc7ccc8c(C9CCC(=O)NC9=O)noc8c7C5)C6)cc4)n3)C2)C1=O ; CN1CCN(C2CCCN(c3cnc(C(N)=O)c(Nc4ccc(C5CCC6(CC5)CN([C@H]5Cc7ccc8c(C9CCC(=O)NC9=O)noc8c7C5)C6)cc4)n3)C2)C1=O ; CN1CCN(C2CCCN(c3cnc(C(N)=O)c(Nc4ccc(C5CCC6(CC5)CN([C@@H]5Cc7ccc8c(C9CCC(=O)NC9=O)noc8c7C5)C6)cc4)n3)C2)C1=O]

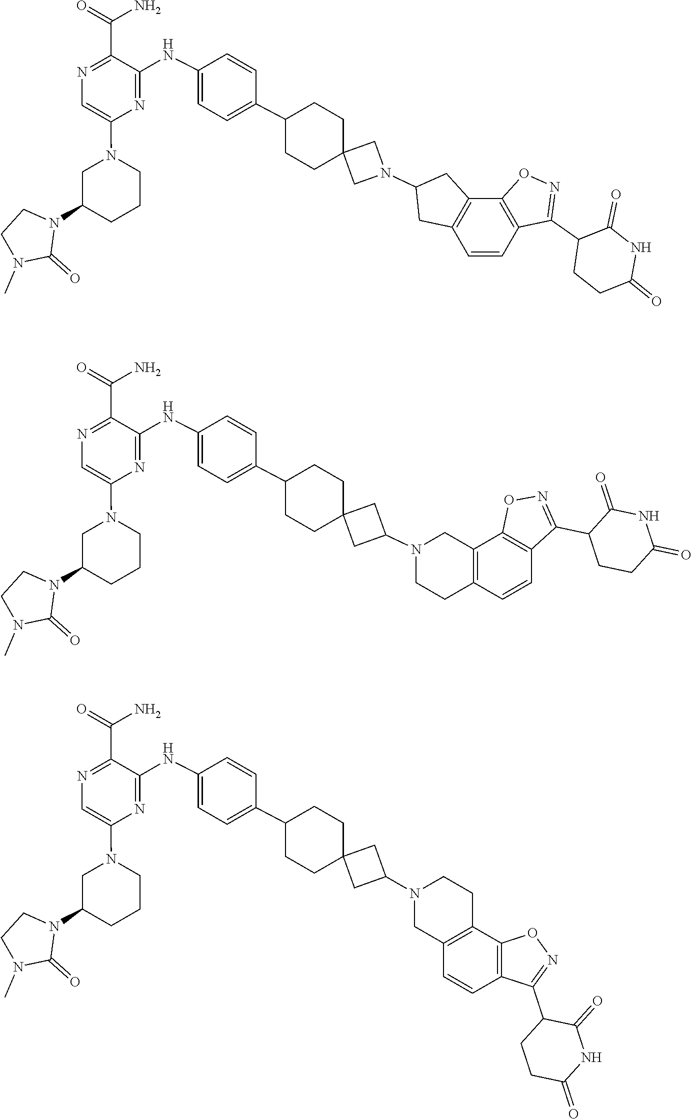 [SMILES: CN1CCN([C@@H]2CCCN(c3cnc(C(N)=O)c(Nc4ccc(C5CCC6(CC5)CN(C5Cc7ccc8c(C9CCC(=O)NC9=O)noc8c7C5)C6)cc4)n3)C2)C1=O ; CN1CCN([C@@H]2CCCN(c3cnc(C(N)=O)c(Nc4ccc(C5CCC6(CC5)CC(N5CCc7ccc8c(C9CCC(=O)NC9=O)noc8c7C5)C6)cc4)n3)C2)C1=O ; CN1CCN([C@@H]2CCCN(c3cnc(C(N)=O)c(Nc4ccc(C5CCC6(CC5)CC(N5CCc7c(ccc8c(C9CCC(=O)NC9=O)noc78)C5)C6)cc4)n3)C2)C1=O]

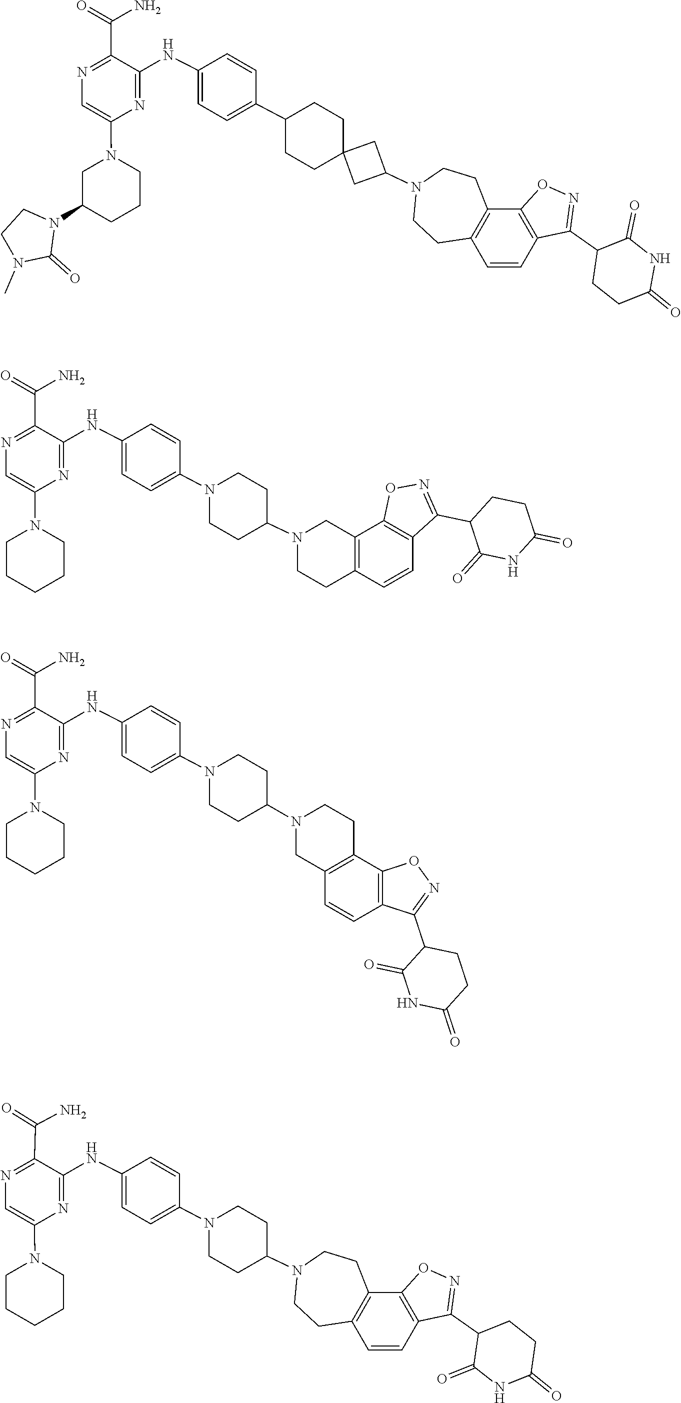 [SMILES: CN1CCN([C@@H]2CCCN(c3cnc(C(N)=O)c(Nc4ccc(C5CCC6(CC5)CC(N5CCc7ccc8c(C9CCC(=O)NC9=O)noc8c7CC5)C6)cc4)n3)C2)C1=O ; NC(=O)c1ncc(N2CCCCC2)nc1Nc1ccc(N2CCC(N3CCc4ccc5c(C6CCC(=O)NC6=O)noc5c4C3)CC2)cc1 ; NC(=O)c1ncc(N2CCCCC2)nc1Nc1ccc(N2CCC(N3CCc4c(ccc5c(C6CCC(=O)NC6=O)noc45)C3)CC2)cc1 ; NC(=O)c1ncc(N2CCCCC2)nc1Nc1ccc(N2CCC(N3CCc4ccc5c(C6CCC(=O)NC6=O)noc5c4CC3)CC2)cc1]

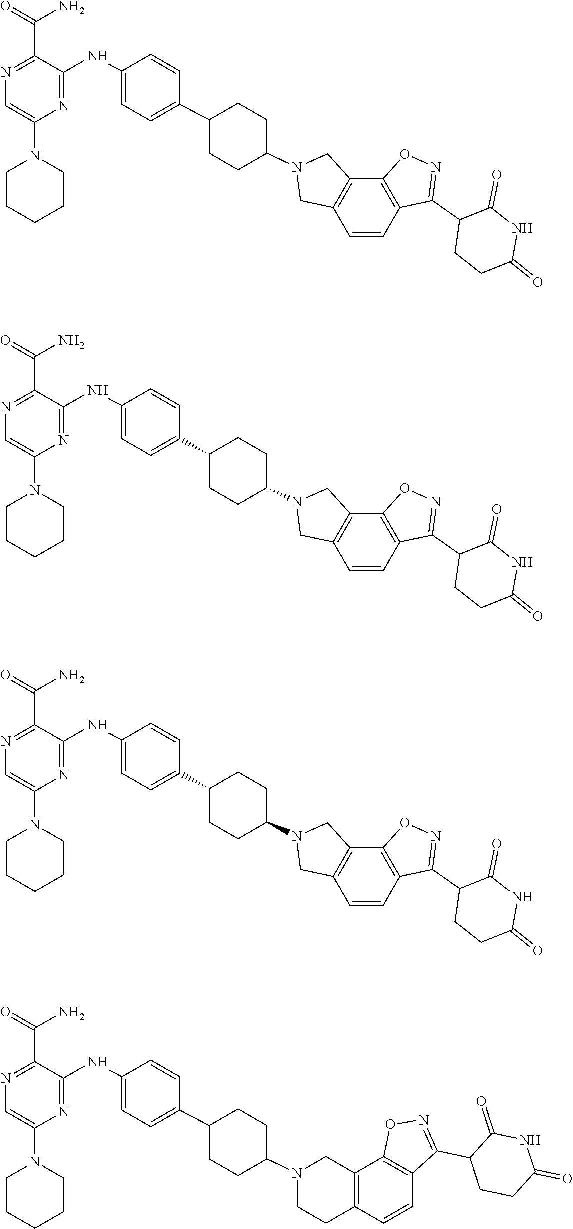 [SMILES: NC(=O)c1ncc(N2CCCCC2)nc1Nc1ccc(C2CCC(N3Cc4ccc5c(C6CCC(=O)NC6=O)noc5c4C3)CC2)cc1 ; NC(=O)c1ncc(N2CCCCC2)nc1Nc1ccc([C@H]2CC[C@@H](N3Cc4ccc5c(C6CCC(=O)NC6=O)noc5c4C3)CC2)cc1 ; NC(=O)c1ncc(N2CCCCC2)nc1Nc1ccc([C@H]2CC[C@H](N3Cc4ccc5c(C6CCC(=O)NC6=O)noc5c4C3)CC2)cc1 ; NC(=O)c1ncc(N2CCCCC2)nc1Nc1ccc(C2CCC(N3CCc4ccc5c(C6CCC(=O)NC6=O)noc5c4C3)CC2)cc1]

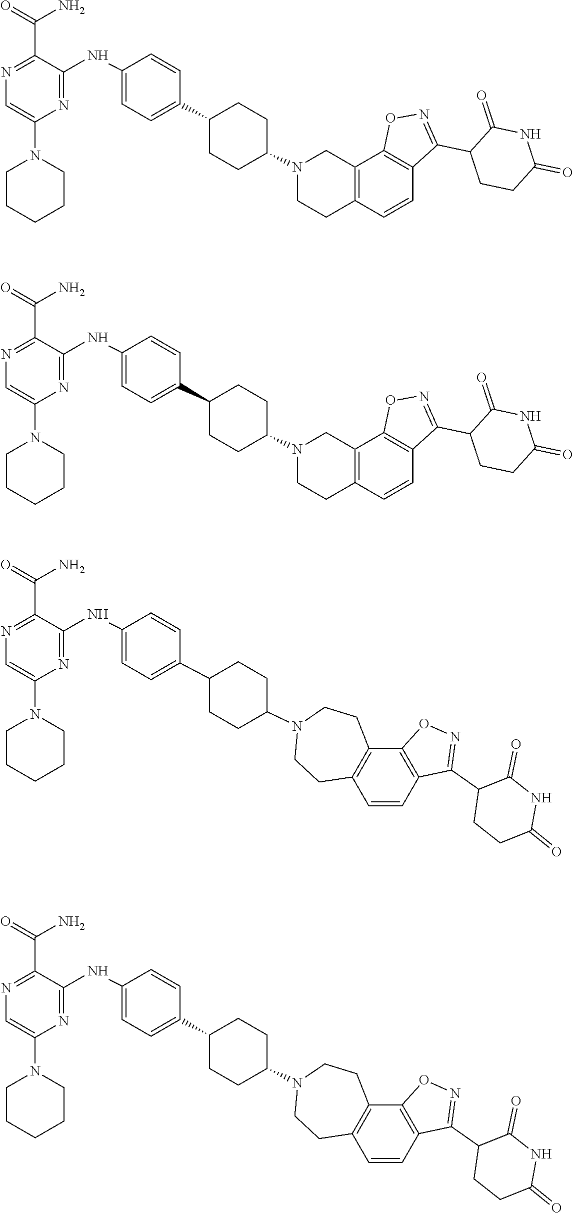 [SMILES: NC(=O)c1ncc(N2CCCCC2)nc1Nc1ccc([C@H]2CC[C@@H](N3CCc4ccc5c(C6CCC(=O)NC6=O)noc5c4C3)CC2)cc1 ; NC(=O)c1ncc(N2CCCCC2)nc1Nc1ccc([C@H]2CC[C@H](N3CCc4ccc5c(C6CCC(=O)NC6=O)noc5c4C3)CC2)cc1 ; NC(=O)c1ncc(N2CCCCC2)nc1Nc1ccc(C2CCC(N3CCc4ccc5c(C6CCC(=O)NC6=O)noc5c4CC3)CC2)cc1 ; NC(=O)c1ncc(N2CCCCC2)nc1Nc1ccc([C@H]2CC[C@@H](N3CCc4ccc5c(C6CCC(=O)NC6=O)noc5c4CC3)CC2)cc1]

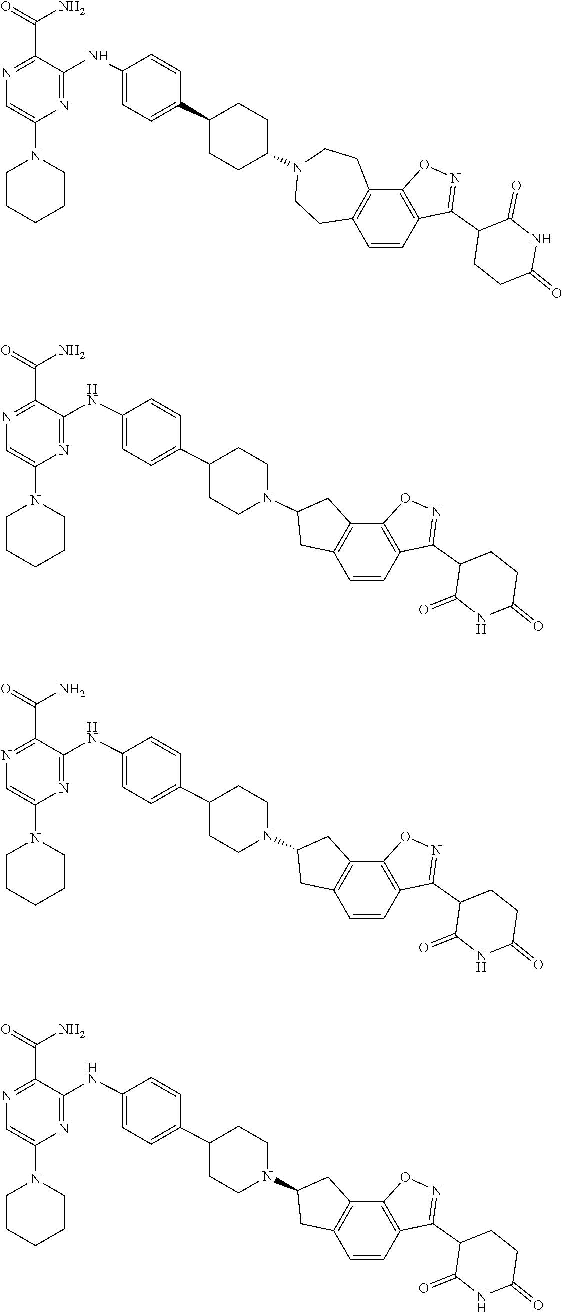 [SMILES: NC(=O)c1ncc(N2CCCCC2)nc1Nc1ccc([C@H]2CC[C@H](N3CCc4ccc5c(C6CCC(=O)NC6=O)noc5c4CC3)CC2)cc1 ; NC(=O)c1ncc(N2CCCCC2)nc1Nc1ccc(C2CCN(C3Cc4ccc5c(C6CCC(=O)NC6=O)noc5c4C3)CC2)cc1 ; NC(=O)c1ncc(N2CCCCC2)nc1Nc1ccc(C2CCN([C@H]3Cc4ccc5c(C6CCC(=O)NC6=O)noc5c4C3)CC2)cc1 ; NC(=O)c1ncc(N2CCCCC2)nc1Nc1ccc(C2CCN([C@@H]3Cc4ccc5c(C6CCC(=O)NC6=O)noc5c4C3)CC2)cc1]

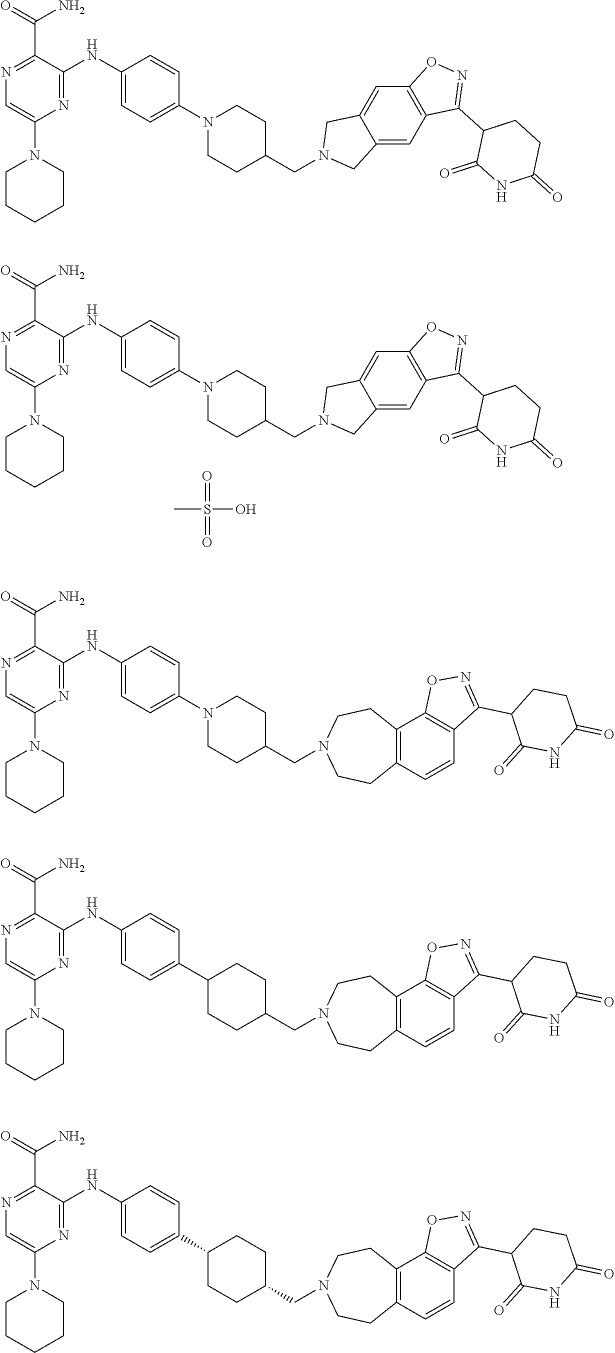

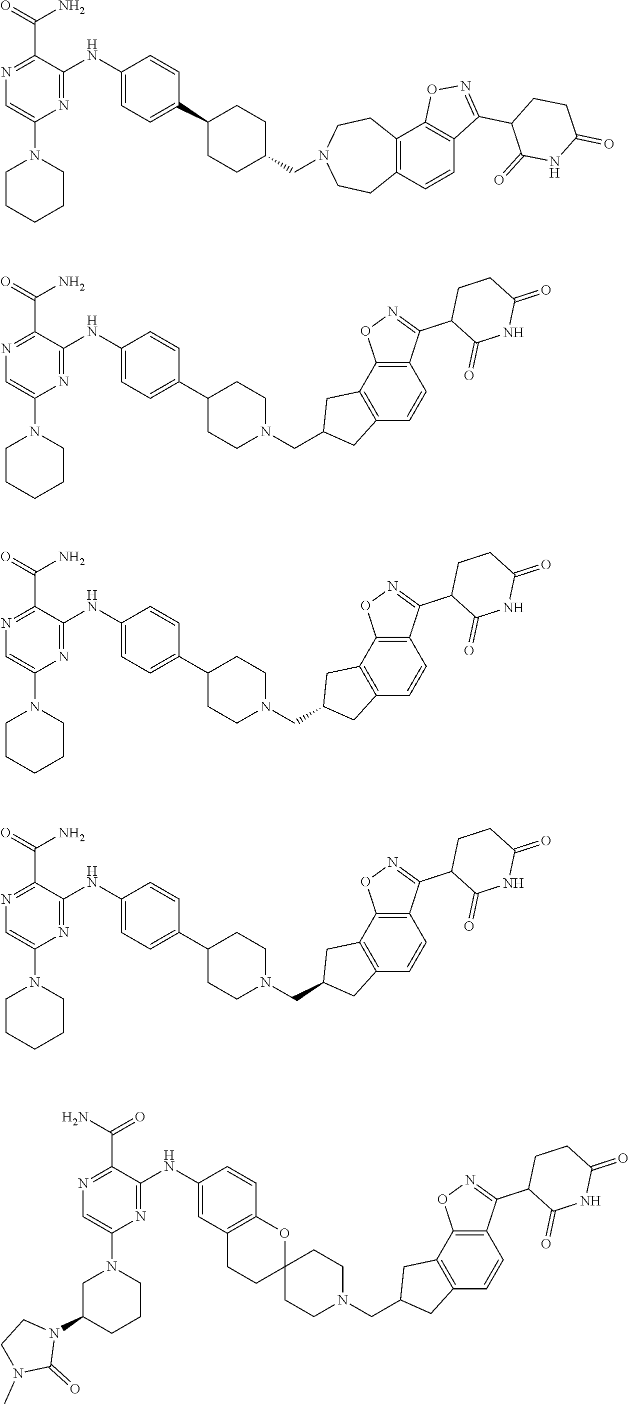 [SMILES: NC(=O)c1ncc(N2CCCCC2)nc1Nc1ccc([C@H]2CC[C@H](CN3CCc4ccc5c(C6CCC(=O)NC6=O)noc5c4CC3)CC2)cc1 ; NC(=O)c1ncc(N2CCCCC2)nc1Nc1ccc(C2CCN(CC3Cc4ccc5c(C6CCC(=O)NC6=O)noc5c4C3)CC2)cc1 ; NC(=O)c1ncc(N2CCCCC2)nc1Nc1ccc(C2CCN(C[C@H]3Cc4ccc5c(C6CCC(=O)NC6=O)noc5c4C3)CC2)cc1 ; NC(=O)c1ncc(N2CCCCC2)nc1Nc1ccc(C2CCN(C[C@@H]3Cc4ccc5c(C6CCC(=O)NC6=O)noc5c4C3)CC2)cc1 ; CN1CCN([C@@H]2CCCN(c3cnc(C(N)=O)c(Nc4ccc5c(c4)CCC4(CCN(CC6Cc7ccc8c(C9CCC(=O)NC9=O)noc8c7C6)CC4)O5)n3)C2)C1=O]

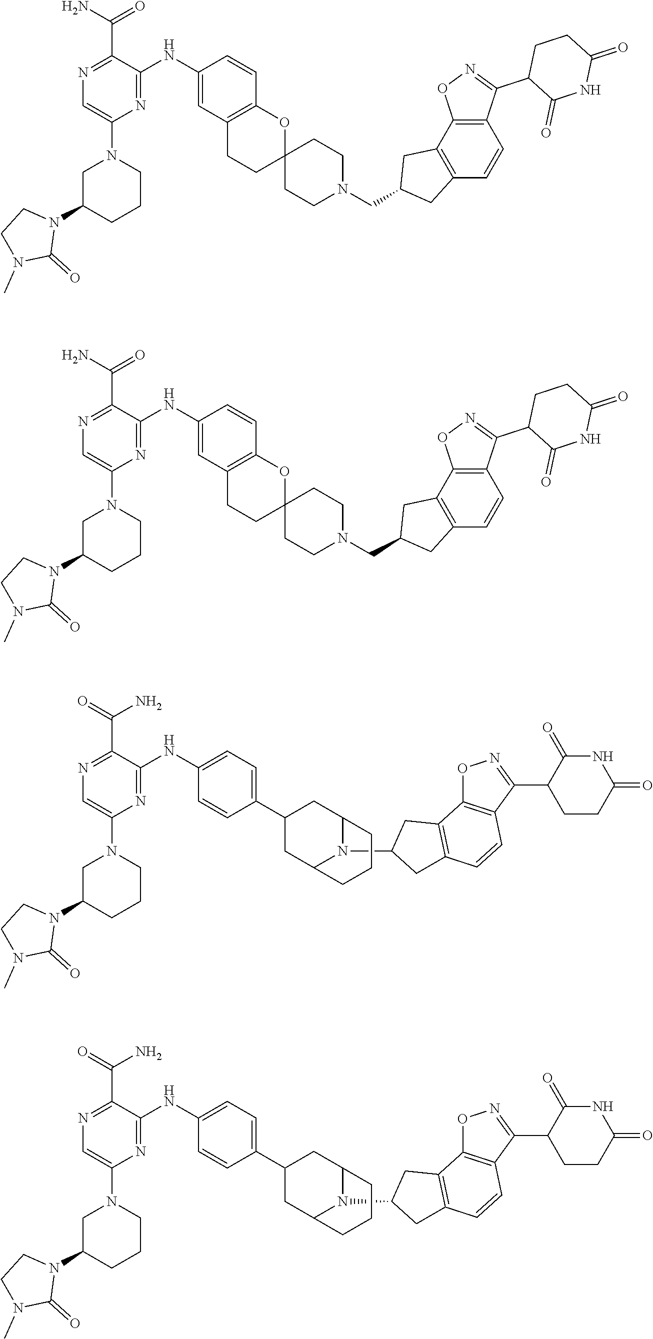 [SMILES: CN1CCN([C@@H]2CCCN(c3cnc(C(N)=O)c(Nc4ccc5c(c4)CCC4(CCN(C[C@H]6Cc7ccc8c(C9CCC(=O)NC9=O)noc8c7C6)CC4)O5)n3)C2)C1=O ; CN1CCN([C@@H]2CCCN(c3cnc(C(N)=O)c(Nc4ccc5c(c4)CCC4(CCN(C[C@@H]6Cc7ccc8c(C9CCC(=O)NC9=O)noc8c7C6)CC4)O5)n3)C2)C1=O ; CN1CCN([C@@H]2CCCN(c3cnc(C(N)=O)c(Nc4ccc(C5CC6CCCC(C5)N6C5Cc6ccc7c(C8CCC(=O)NC8=O)noc7c6C5)cc4)n3)C2)C1=O ; CN1CCN([C@@H]2CCCN(c3cnc(C(N)=O)c(Nc4ccc(C5CC6CCCC(C5)N6[C@H]5Cc6ccc7c(C8CCC(=O)NC8=O)noc7c6C5)cc4)n3)C2)C1=O]

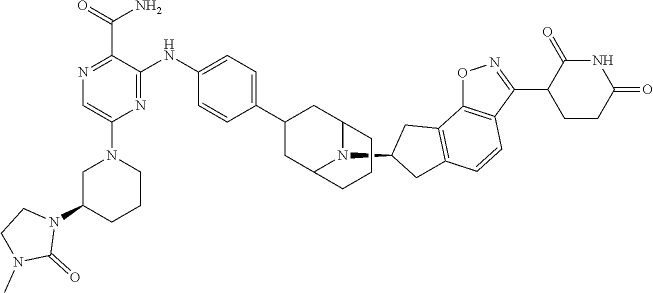 [SMILES: CN1CCN([C@@H]2CCCN(c3cnc(C(N)=O)c(Nc4ccc(C5CC6CCCC(C5)N6[C@@H]5Cc6ccc7c(C8CCC(=O)NC8=O)noc7c6C5)cc4)n3)C2)C1=O]

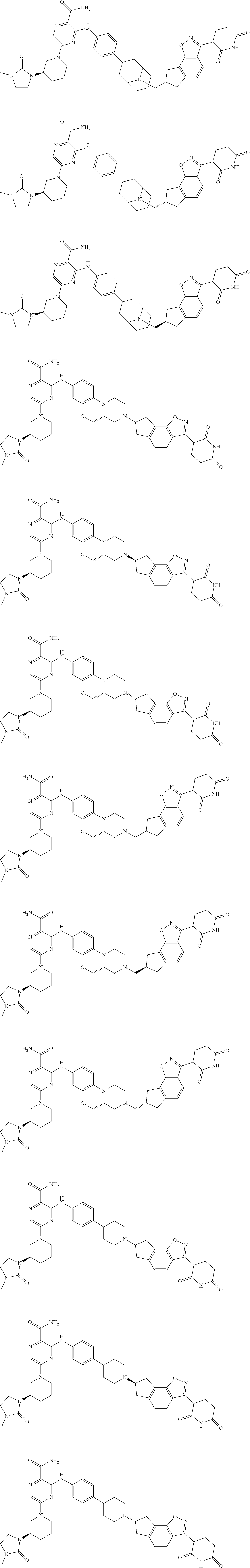

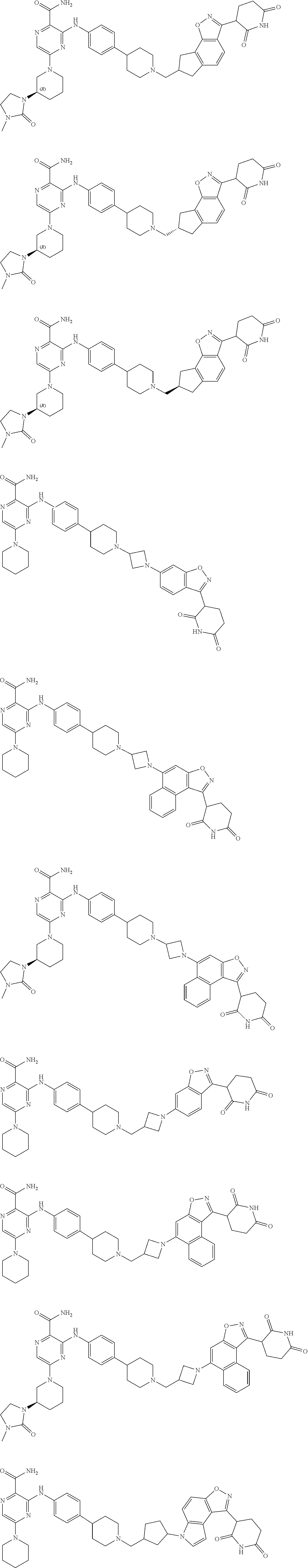

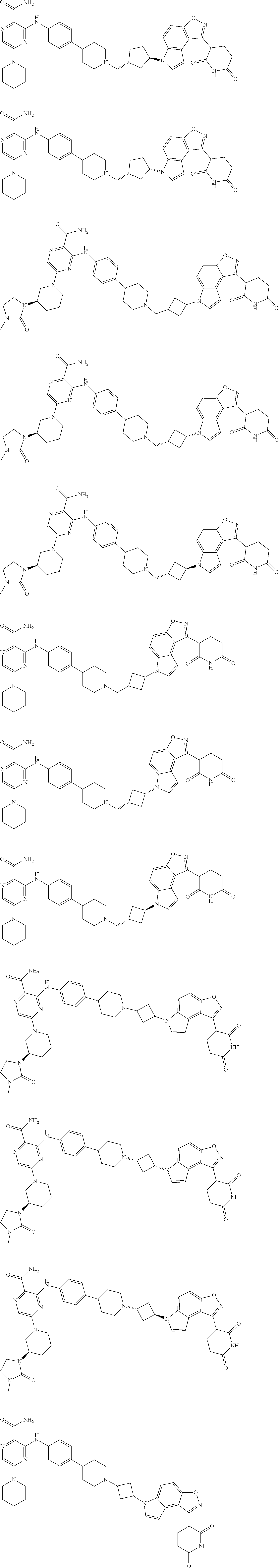

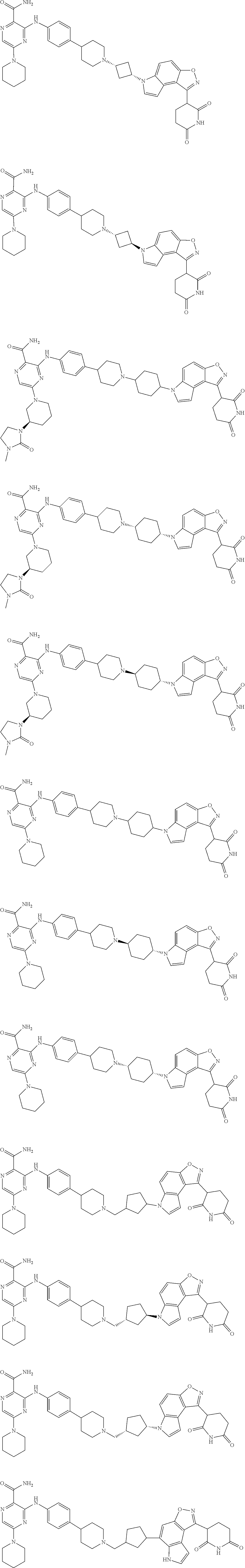

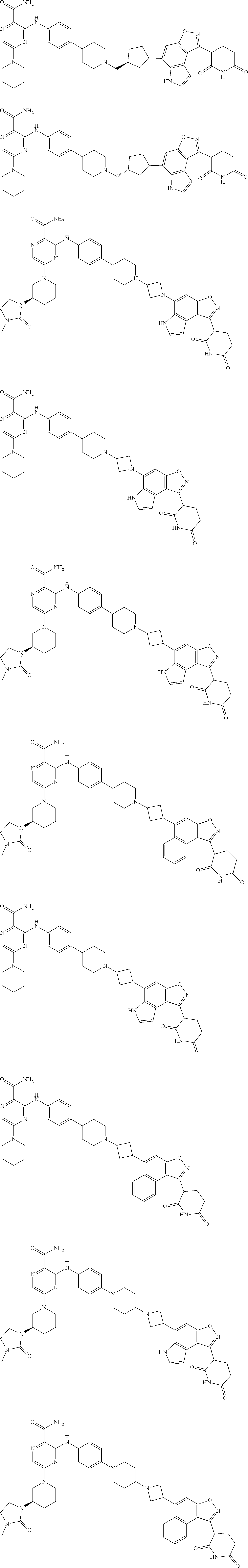

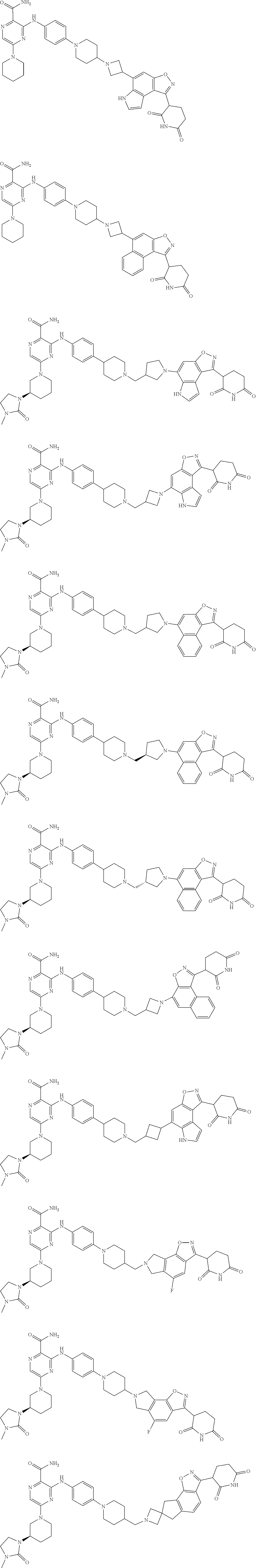

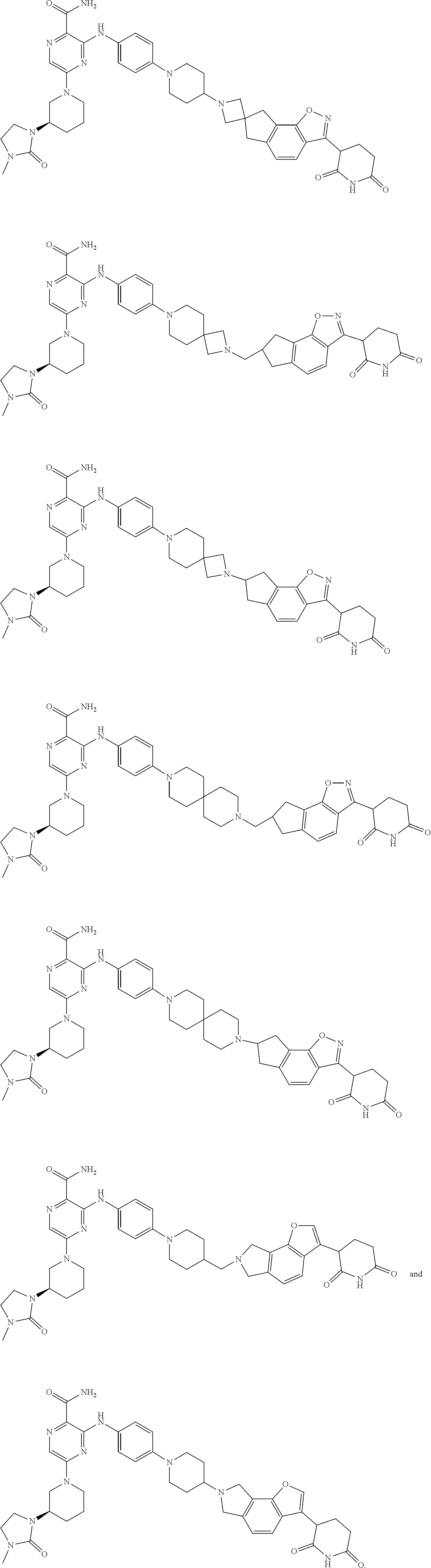

58. A compound of formula I″′-1a or I″′-2a, formula I″′-1c or I″′-2d, a moiety, a stereoisomer thereof, a derivative thereof, or a pharmaceutically acceptable salt thereof: 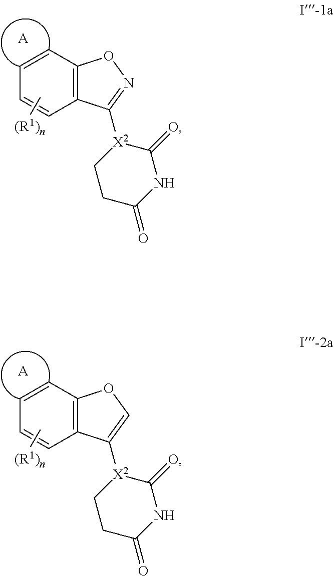
wherein,
ring A is selected from the group consisting of C₅₋₁₀ cycloalkenyl and 5- to 10-membered heterocycloalkenyl;
n is selected from the group consisting of 0, 1, 2, and 3;
each R¹ is independently selected from the group consisting of halogen, —OH, —NH₂, —CN, ═O, C₁₋₄ alkoxy, —CHO, C₃₋₆ cycloalkyl, 3- to 10-membered heterocycloalkyl, and C₁₋₄ alkyl, wherein the C₃₋₆ cycloalkyl, 3- to 10-membered heterocycloalkyl, or C₁₋₄ alkyl is optionally substituted with halogen, —OH, —NH₂, or C₁₋₄ alkyl-OH; or, each R¹ is independently selected from the group consisting of halogen, —OH, —NH₂, —CN, ═O, C₁₋₄ alkoxy, —CHO, C₃₋₆ cycloalkyl, and C₁₋₄ alkyl, wherein the C₃₋₆ cycloalkyl or C₁₋₄ alkyl is optionally substituted with halogen, —OH, —NH₂, or C₁₋₄ alkyl-OH;
X² is selected from the group consisting of CH and N; or 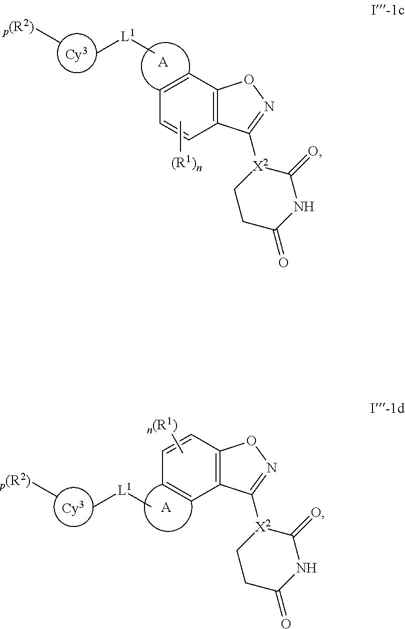
wherein,
ring A is absent or selected from the group consisting of C₅₋₁₀ cycloalkenyl, 5- to 10-membered heterocycloalkenyl, phenyl, and 5- to 6-membered heteroaryl;
n is selected from the group consisting of 0, 1, 2, and 3;
each R¹ is independently selected from the group consisting of halogen, —OH, —NH₂, —CN, C₁₋₄ alkyl, C₁₋₄ alkoxy, and C₁₋₄ haloalkyl;
X² is selected from the group consisting of CH and N;
L¹ is selected from C₀₋₃ alkylene;
Cy³ is selected from the group consisting of C₃₋₈ cycloalkyl and 3- to 8-membered heterocycloalkyl;
R² is selected from the group consisting of —CHO, OH, SH, NH₂, COOH, and C₁₋₆ alkyl substituted with one or more SH, OH, or NH₂; and
p is selected from the group consisting of 0, 1, 2, and 3.

59. The compound, the stereoisomer thereof, the derivative thereof, or the pharmaceutically acceptable salt thereof according to claim 58, wherein in the compound of formula I″′-1a or I″′-2a, each R¹ is independently selected from the group consisting of halogen, —OH, —NH₂, —CN, ═O, C₁₋₃ alkoxy, —CHO, C₃₋₄ cycloalkyl, and C₁₋₃ alkyl, wherein the C₃₋₄ cycloalkyl or C₁₋₃ alkyl is optionally substituted with halogen, —OH, —NH₂, or C₁₋₃ alkyl-OH; or
each R¹ is independently selected from the group consisting of halogen, —OH, —NH₂, —CN, ═O, methoxy, —CHO, cyclobutyl, and methyl, wherein the cyclobutyl or methyl is optionally substituted with halogen, —OH, —NH₂, or CH₂OH; or
each R¹ is independently selected from the group consisting of F, —OH, —NH₂, —CH₂OH, ═O, —CHO, —CH₂NH₂, and 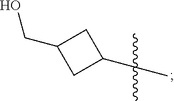 [SMILES: *C1CC(CO)C1]
optionally, wherein the compound of formula I″′-1a or I″′-2a is selected from a compound of formula I″′-1 or I″′-2, respectively: 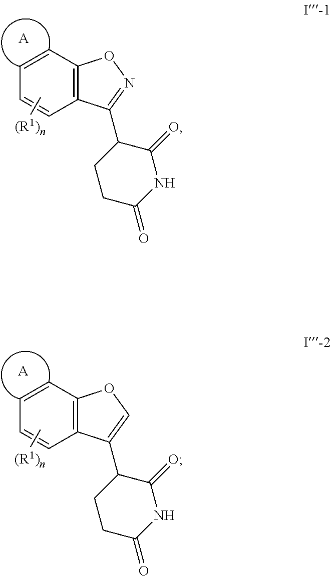
optionally, wherein in the compound of formula I″′-1a or I″′-2a, ring A is selected from the group consisting of C₅₋₈ cycloalkenyl and 5- to 10-membered heterocycloalkenyl; or ring A is selected from the group consisting of C₅₋₇ cycloalkenyl and 5- to 10-membered heterocycloalkenyl; or ring A is selected from the group consisting of C₅₋₆ cycloalkenyl and 5- to 10-membered heterocycloalkenyl; or ring A is selected from the group consisting of C₅₋₆ cycloalkenyl and 5- to 9-membered heterocycloalkenyl; or
ring A is selected from the group consisting of C₅ cycloalkenyl, C₆ cycloalkenyl, and 5-, 6-, 7-, 8-, or 9-membered heterocycloalkenyl; or
ring A is selected from the group consisting of cyclopentenyl, bicyclohexenyl, dihydropyrrolyl, tetrahydropyridinyl, tetrahydroazepinyl, dihydrooxazinyl, azaspirooctenyl, and azaspirononenyl; or
ring A is selected from the group consisting of 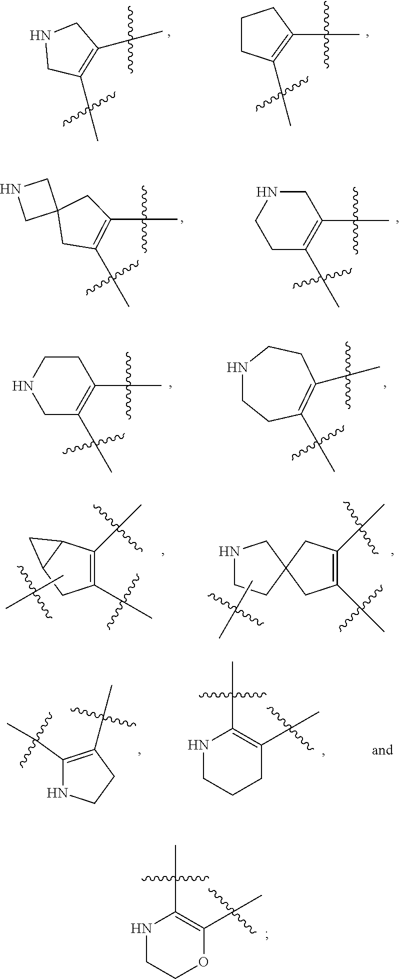
or
ring A is selected from the group consisting of 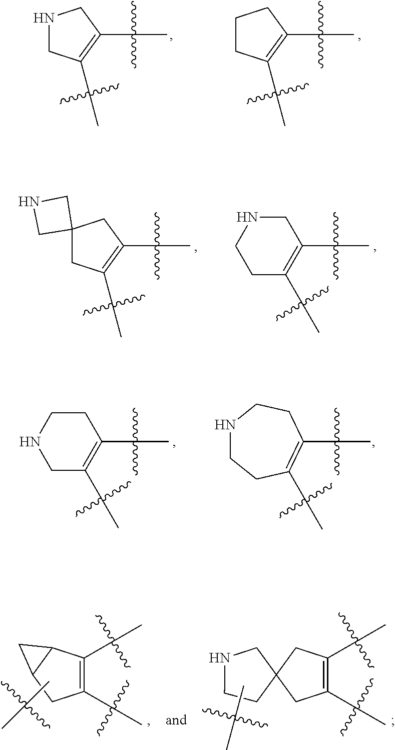
optionally, in the compound of formula I″′-1c or I″′-2d, ring A is selected from the group consisting of C₅₋₉ cycloalkenyl, 5- to 9-membered heterocycloalkenyl, phenyl, and 5- to 6-membered heteroaryl; ring A is selected from the group consisting of C₅₋₇ cycloalkenyl, 5- to 9-membered heterocycloalkenyl, phenyl, and 5- to 6-membered heteroaryl; ring A is selected from the group consisting of 5- to 9-membered heterocycloalkenyl, phenyl, and 5- to 6-membered heteroaryl; or
ring A is selected from the group consisting of 5- to 6-membered heterocycloalkenyl, phenyl, and 5-membered heteroaryl; or
ring A is selected from the group consisting of dihydropyrrolyl, tetrahydropyridinyl, dihydrooxazinyl, phenyl, pyrrolyl, pyrazolyl, and furanyl;
optionally, wherein in the compound of formula I″′-1c or I″′-2d, the moiety 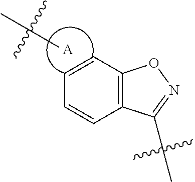
is selected from the group consisting of 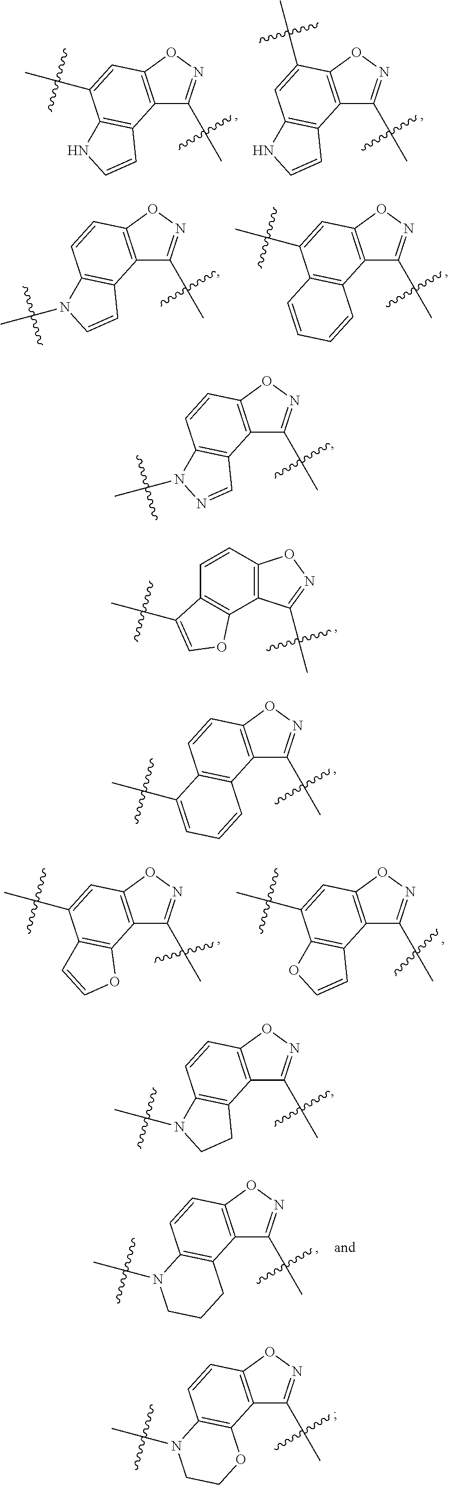
or,
wherein in the compound of formula I″′-1c or I″′-2d, L¹ is selected from the group consisting of a bond and —CH₂—;
or,
Cy³ is selected from the group consisting of C₃₋₆ cycloalkyl and 4- to 6-membered heterocycloalkyl; or
Cy³ is selected from the group consisting of C₄₋₆ cycloalkyl, 4-membered heterocycloalkyl, and 6-membered heterocycloalkyl; or
Cy³ is selected from the group consisting of cyclobutyl, cyclopentyl, cyclohexyl, azetidinyl, and piperidinyl;
or,
wherein in the compound of formula I″′-1c or I″′-2d, R² is selected from the group consisting of —CHO, OH, and C₁₋₃ alkyl substituted with one or more OH or NH₂; or
R² is selected from the group consisting of —CHO, OH, and methyl substituted with one or more OH; or
R² is selected from the group consisting of —CHO, OH, and —CH₂OH;
optionally, p is selected from the group consisting of 0, 1 and 2; or
p is selected from the group consisting of 0 and 1; or
p is selected from 0; or, p is selected from 1;
optionally, wherein in the compound of formula I″′-1c or I″′-2d, the structural moiety 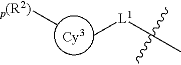
is selected from the group consisting of

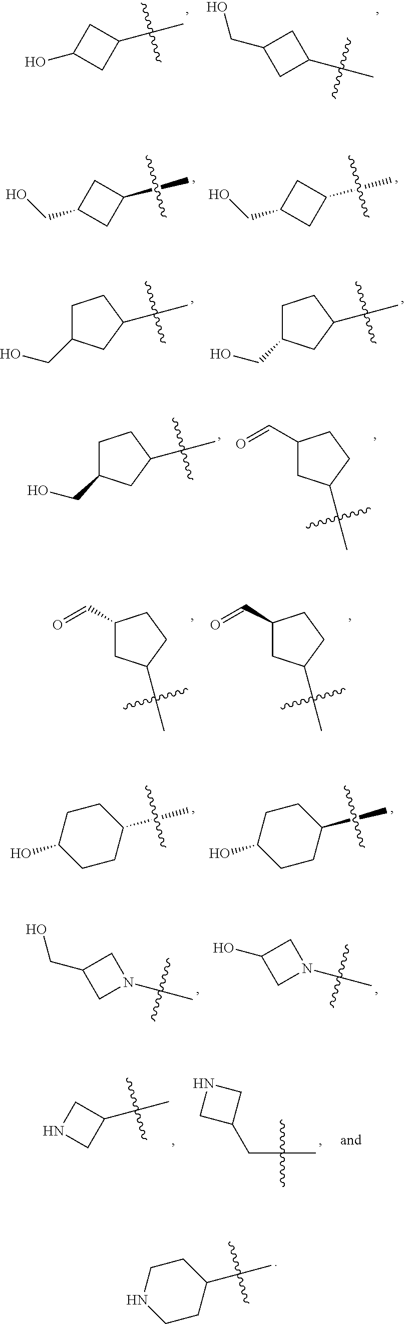

60. The compound, the moiety, the stereoisomer thereof, the derivative thereof, or the pharmaceutically acceptable salt thereof according to claim 58, wherein the compound of formula I″′-1a or I″′-2a is selected from the group consisting of: 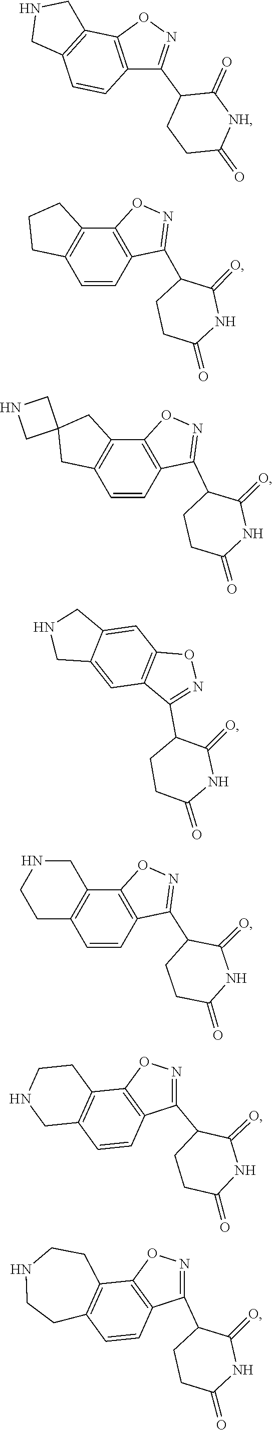 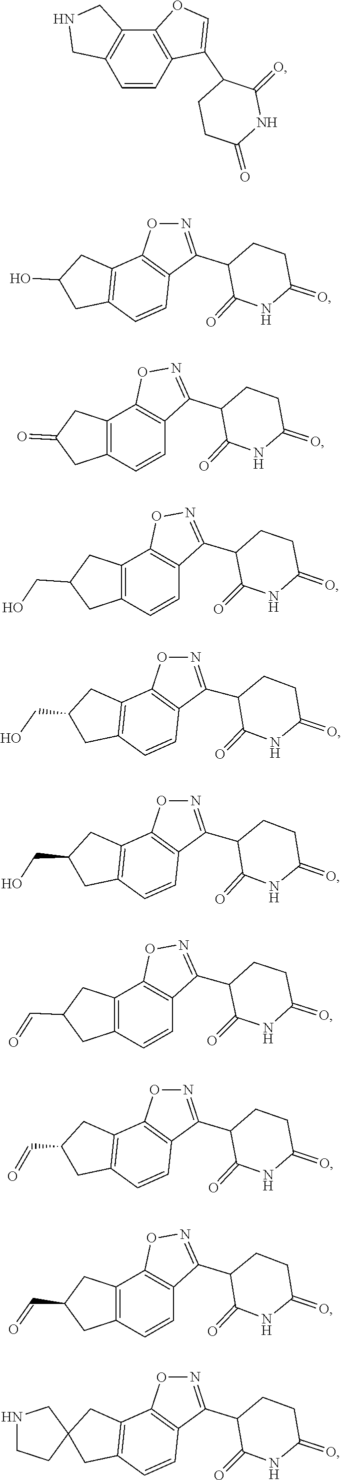 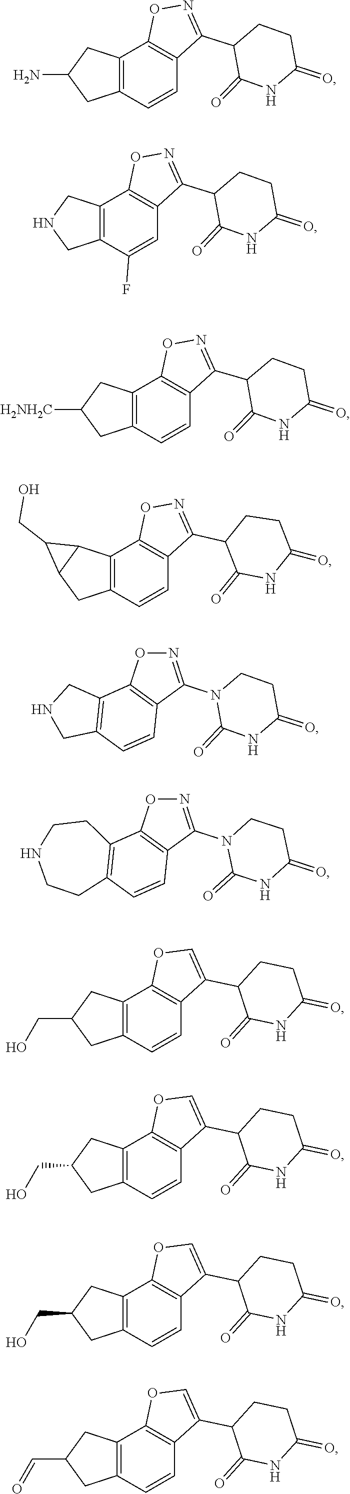 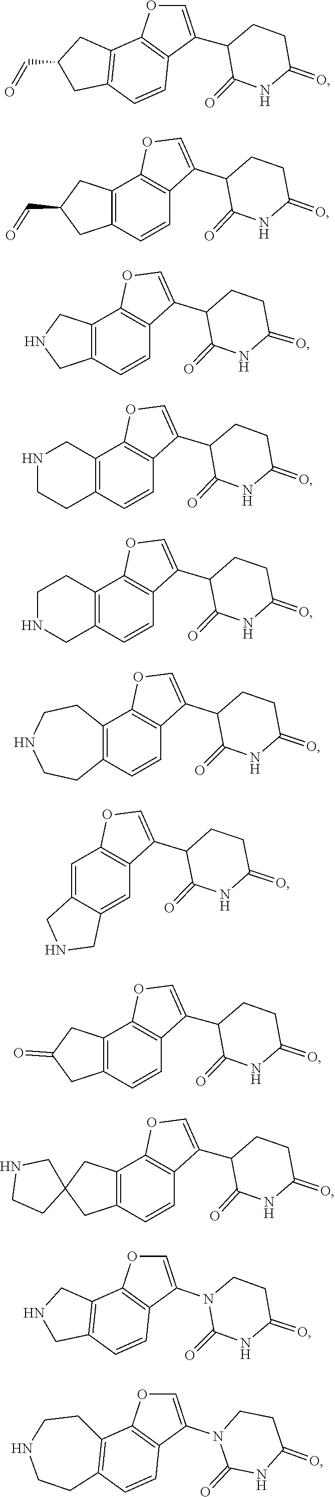 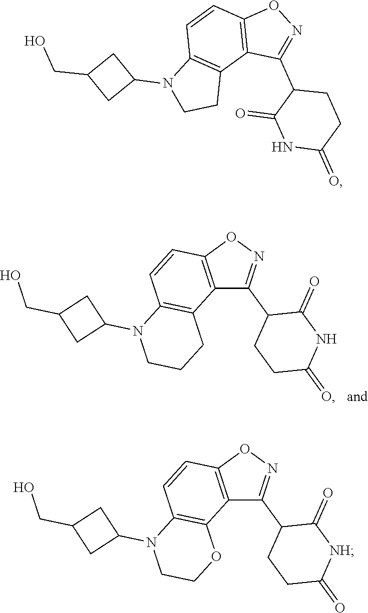
or,
wherein the compound of formula I″′-1c or I″′-2d is selected from the group consisting of,

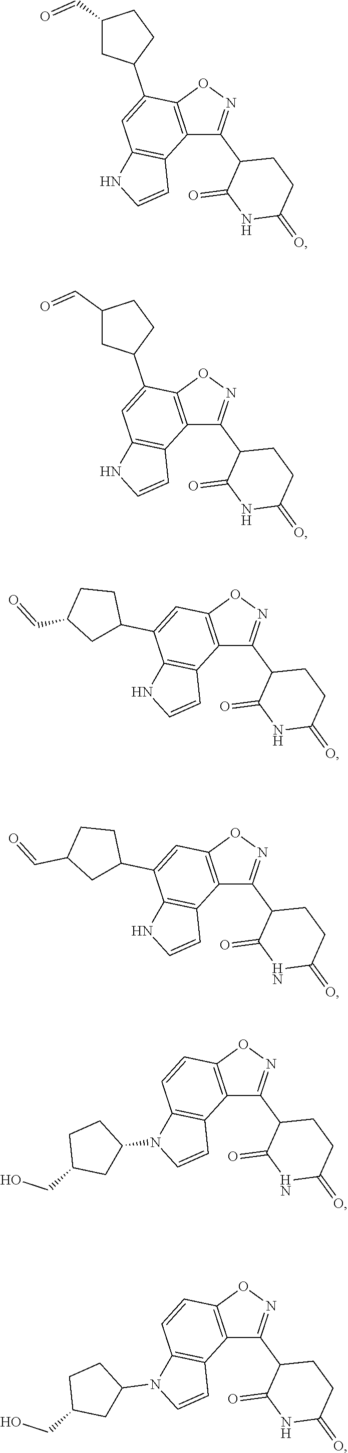 [SMILES: O=C[C@@H]1CCC(c2cc3[nH]ccc3c3c(C4CCC(=O)NC4=O)noc23)C1 ; O=CC1CCC(c2cc3[nH]ccc3c3c(C4CCC(=O)NC4=O)noc23)C1 ; O=C[C@@H]1CCC(c2cc3onc(C4CCC(=O)NC4=O)c3c3cc[nH]c23)C1 ; O=CC1CCC(c2cc3onc(C4CCC(=O)NC4=O)c3c3cc[nH]c23)C1 ; O=C1CCC(c2noc3ccc4c(ccn4[C@H]4CC[C@@H](CO)C4)c23)C(=O)N1 ; O=C1CCC(c2noc3ccc4c(ccn4C4CC[C@@H](CO)C4)c23)C(=O)N1]

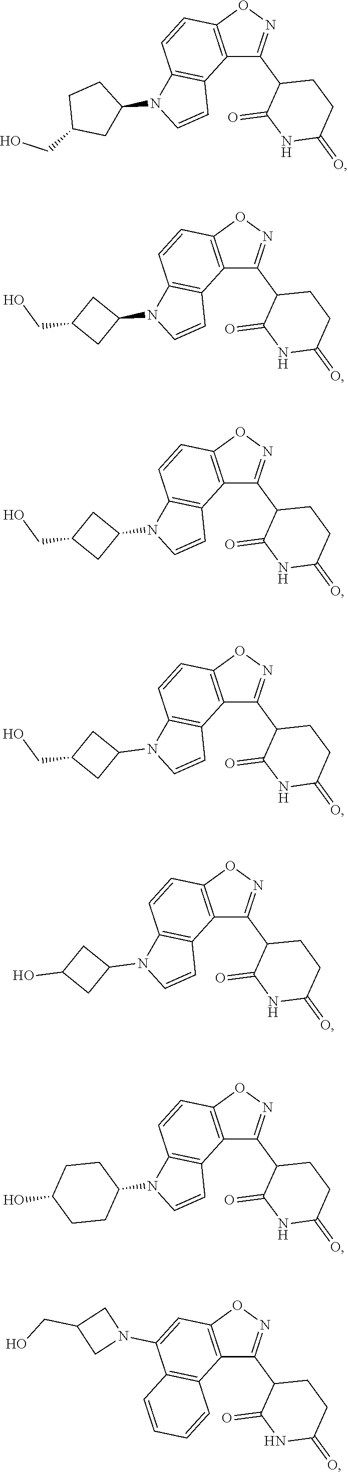

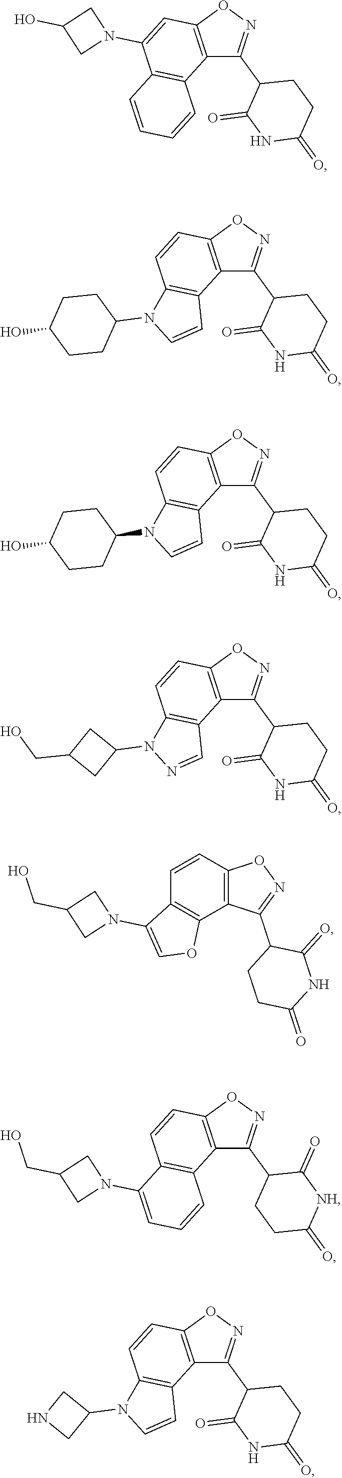

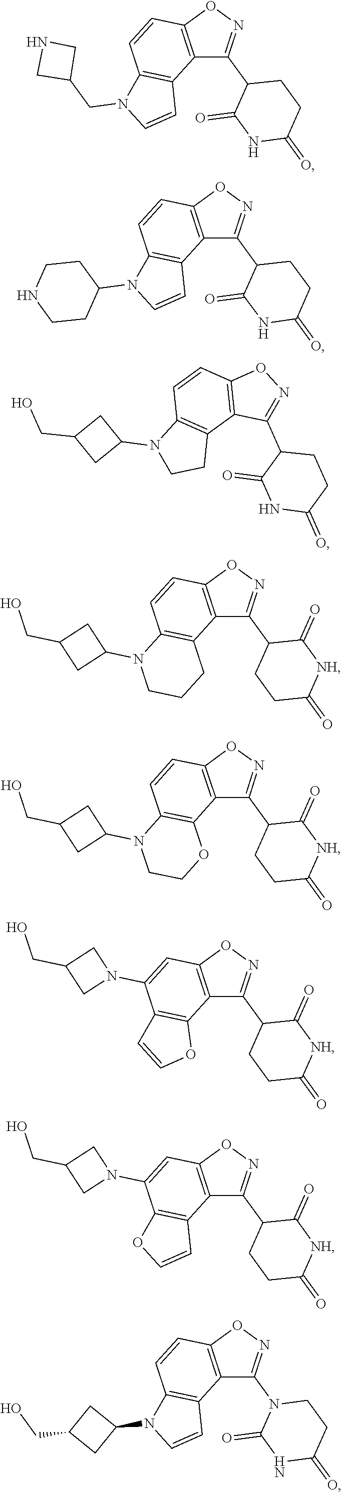

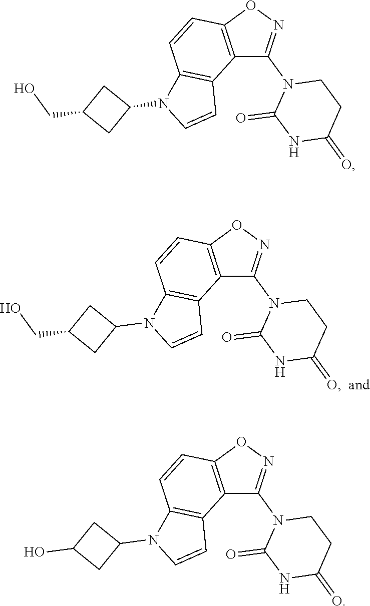

61. A pharmaceutical composition, comprising the compound, the moiety, the stereoisomer thereof, the derivative thereof, or the pharmaceutically acceptable salt thereof according to claim 45.

62. A method of treating or preventing a disorder treated by degrading a target protein that binds to a targeting ligand, preventing or treating a disorder treated by binding to cereblon protein in vivo, or preventing or treating a BTK-related disease in a mammal in need thereof a therapeutically effective amount of the pharmaceutically acceptable salt thereof according to claim 45 or a pharmaceutical composition comprising the same,
optionally, the BTK-related disease is selected from the group consisting of disorders treated by degrading a protein that binds to a BTK target protein ligand;
optionally, the BTK-related disease is selected from the group consisting of disorders treated by binding to cereblon protein in vivo; and
optionally, the disease is selected from the group consisting of autoimmune diseases, inflammatory diseases, and cancer.
